(19) United States
(12) Patent Application Publication
(10) Pub. No.: US 2022/0257796 A1
(43) Pub. Date: Aug. 18, 2022

(54) RECOMBINANT AD35 VECTORS AND RELATED GENE THERAPY IMPROVEMENTS

(71) Applicants: Fred Hutchinson Cancer Research Center, Seattle, WA (US); University of Washington, Seattle, WA (US)

(72) Inventors: Hans-Peter Kiem, Seattle, WA (US); Andre Lieber, Seattle, WA (US); Chang Li, Seattle, WA (US); Hongjie Wang, Seattle, WA (US)

(73) Assignees: Fred Hutchinson Cancer Research Center, Seattle, WA (US); University of Washington, Seattle, WA (US)

(21) Appl. No.: 17/618,774

(22) PCT Filed: Jul. 2, 2020
(86) PCT No.: PCT/US2020/040756

§ 371 Date: Dec. 13, 2021

Related U.S. Application Data
(60) Provisional application No. 62/869,907, filed on Jul. 2, 2019.
(60) Provisional application No. 62/935,507, filed on Nov. 14, 2019.
(60) Provisional application No. 63/009,385, filed on Apr. 13, 2020.

(51) Int. Cl. A61K 48/00; C12N 15/11; C12N 15/113; C12N 15/90; C12N 15/86; C12N 9/22

(52) U.S. Cl. CPC A61K 48/0091; C12N 15/111; C12N 15/113; C12N 15/907; C12N 2800/90; C12N 9/22; C12N 2310/20; C12N 2710/10343; C12N 2800/80; C12N 15/86

(57) ABSTRACT

The present disclosure provides, among other things, helper-dependent adenoviral serotype 35 (Ad35) vectors. In various embodiments, helper-dependent Ad35 vectors can be used to deliver a therapeutic payload to a subject in need thereof. Exemplary payloads can encode replacement proteins, antibodies, CARs, TCRs, small RNAs, and genome editing systems. In certain embodiments, a helper-dependent Ad35 vector is engineered for integration of a payload into a host cell genome. The present disclosure further includes methods of gene therapy that include administration of a helper-dependent Ad35 vector to a subject in need thereof.

### CROSS-REFERENCE TO RELATED APPLICATIONS

This is the U.S. National Phase of International Patent Application no. PCT/US2020/040756, filed Jul. 2, 2020, which claims priority to and the benefit of the earlier filing date of U.S. Provisional Application No. 62/869,907, filed Jul. 2, 2019, U.S. Provisional Application No. 62/935,507, filed Nov. 14, 2019, and U.S. Provisional Application No. 63/009,385, filed Apr. 13, 2020, the disclosure of each of which is hereby incorporated by reference in its entirety.

### GOVERNMENT SUPPORT

This invention was made with government support under grant numbers HL130040, HL141781, CA204036, HL128288, and HL136135 awarded by the National Institutes of Health. The government has certain rights in the invention.

### STATEMENT REGARDING SEQUENCE LISTING

The Sequence Listing associated with this application is provided in text format in lieu of a paper copy and is hereby incorporated by reference into the specification. The name of the text file containing the Sequence Listing is 2LX4116.txt. The text file is 980 KB, was created on Dec. 10, 2021, and is being submitted electronically via EFS-Web.

### BACKGROUND

Many medical conditions are caused by genetic mutation and/or are treatable, at least in part, by gene therapy. Such conditions include, for example, hemoglobinopathies, immune deficiencies, and cancers. Genetic disorders known as hemoglobinopathies are among the most prevalent types of genetic disorders worldwide, with significantly reduced survival rates among patients born in underdeveloped countries. Examples of hemoglobinopathies include sickle-cell disease and thalassemia. Immune deficiencies can be primary or secondary. More than 80 primary immune deficiency diseases are recognized by the World Health Organization. Prophylactic and therapeutic treatments for medical conditions caused by genetic mutation and/or treatable, at least in part, by gene therapy are needed.

### SUMMARY

Gene therapy can treat many conditions that have a genetic component, including without limitation hemoglobinopathies, immune deficiencies, and cancers. While molecular biology includes various tools for genetic engineering, application of those tools in the gene therapy context, e.g., ex vivo and in vivo, raises new opportunities and challenges, relating at least in part to development of genetic constructs for use in gene therapy vectors, as well as development of the vectors themselves.

The present disclosure includes, among other things, adenoviral vectors and adenoviral genomes (e.g., “recombinant” or “engineered” adenoviral vectors and adenoviral genomes) for expression of base editors in target cells. The present disclosure includes, among other things, adenoviral vectors and adenoviral genomes for expression of a CRISPR system including CRISPR enzyme that is a CRISPR-associated RNA-guided endonuclease and/or a guide RNA (gRNA) in target cells, optionally wherein expression of at least one component of the CRISPR system is self-inactivating. The present disclosure includes, among other things, adenoviral vectors and adenoviral genomes for expression of a base editing system including base editing enzyme and/or a guide RNA (gRNA) in target cells, optionally wherein expression of at least one component of the base editing system is self-inactivating. The present disclosure includes, among other things, adenoviral vectors and adenoviral genomes that include a regulatory sequence that directs expression of an expression product (e.g., a therapeutic expression product) in target cells, where the regulatory sequence includes an miRNA binding site or where the regulatory sequence includes a β-globin locus control region (LCR), such as a β-globin Long LCR. The present disclosure includes, among other things, combination adenoviral vectors and adenoviral genomes that express a plurality of therapeutic expression products in target cells, e.g., therapeutic expression products that together contribute to treatment of a disease or condition. The present disclosure includes, among other things, adenoviral vectors and adenoviral genomes for integration into a target cell genome of a payload including a β-globin Long LCR. The present disclosure includes, among other things, adenoviral vectors, and adenoviral genomes thereof, that have reduced immunogenicity relative to certain existing vectors (e.g., relative to Ad5 vectors). The present disclosure includes, among other things, Ad35 adenoviral vectors, Ad35 adenoviral genomes, HDAd35 adenoviral vectors, HDAd35 adenoviral genomes, support vectors, support genomes, Ad35 helper vectors, and ad Ad35 helper genomes, where HDAd35 vectors can have reduced immunogenicity relative to certain existing vectors (e.g., relative to Ad5 vectors or Ad5/35 vectors).

The current disclosure describes, among other things, recombinant Ad35 vectors targeting CD46 for in vivo gene editing of hematopoietic stem cells and related gene therapy improvements. In particular embodiments of presently disclosed vector designs, all proteins are derived from serotype 35. In particular embodiments of Ad35 vectors described herein, no viral genes remain in the vector. In particular embodiments, the ITR and packaging sequence are derived from Ad35. In particular embodiments, the Ad35 delivery vector has all viral protein encoding genes removed and replaced with components associated with a therapeutic use.

In particular embodiments, the Ad35 vector is helper-dependent, and the current disclosure also provides newly-designed Ad35 helper vectors. Particular embodiments provide optimized ratios of helper-dependent and transgene plasmid to make Ad35.

Related gene therapy improvements described within the current disclosure relate to one or more of: (i) novel mutations of the Ad35 knob protein that increase CD46 binding; (ii) vector features allowing for positive selection of in vivo modified cells; (iii) microRNA control systems that modulate expression of therapeutic proteins within clinically relevant time windows; (iv) use of homology arms to facilitate targeted genomic insertion at defined sites; (v) use of CRISPR to inactivate genomic suppressor regions, allowing increased expression of endogenous genes; (vi) use of mobilization strategies to increase delivery of Ad35 vectors to targeted CD46-expressing cells; (vii) use of mini- or long-form locus control regions to increase gene expression; (viii) use of recombinase systems to increase the size of transposons that can be inserted with transposase systems; (ix) steroid delivery (e.g., glucocorticoids, dexamethasone) before vector delivery; and (x) erythrocytes to generate and secrete therapeutic proteins. Each of these related gene therapy improvements can be practiced with Ad35 vectors described herein and can also be utilized with other viral vector delivery systems. As one example, mutated Ad35 knob proteins that increase CD46 binding can be utilized with a lentiviral or foamy delivery system.

Advances described herein also relate to (i) in vivo HSC transduction/selection technology for SB100x-mediated transgene addition using HDAd5/35++ vectors; (ii) increased HbF reactivation by simultaneously targeting the erythroid bcl11a-enhancer (e.g., to reduce BCL11A expression) and the HBG1/2 promoter regions (to increase expression of γ-globin); (iii) in vivo CRISPR genome engineering; (iv) correction of thalassemia; (v) combination of γ gene addition and reactivation (SB100x system); (vi) self-inactivation of CRISPR/Cas9; (vii) targeted integration using HDAd as donor vectors with self-releasing cassette; (viii) in vivo HSC gene therapy using erythroid cells as a factory for high-level production of a secreted therapeutic protein; (ix) therapeutic approaches to treat cancer (prophylactically and therapeutically); and (x) HDAd35++ vectors.

Certain embodiments relate to mutated knob proteins that increase targeted binding to CD46, allowing for more targeted and specific delivery of therapeutic genes.

Certain embodiments relate to use of homology arms to facilitate targeted genomic insertion, which can be used to provide chromosomal integration into genomic safe harbors, typically open chromatin which allows for higher expression of the transgene levels. As described herein, in particular embodiments, 1.8 b homology arms work well, with 0.8 as a lower limit. Single nucleotide polymorphisms can begin to impact integration at greater than 1.8 b homology arms.

Certain embodiments relate to use of mobilization regimens to alleviate the need for conditioning.

Particular embodiments provide an Ad35 in vivo gene therapy, with (i) an MGMT^{P140K} system that allows for increasing the therapeutic effect by short-term treatment with low-dose O⁶-benzylguanine plus bis-chloroethylnitrosourea, (ii) SB100X transposase-based integration machinery, and (iii) a micro-LCR-driven γ-globin gene.

Particular embodiments include an Ad35 adenovirus vector (HDAd-comb) including (i) a CRISPR/Cas9 cassette targeting the BCL11A binding site within the HBG1/2 promoters to reverse suppression of endogenous genes, (ii) a γ-globin gene cassette driven by a 5 kb β-globin mini-LCR, and an EF1α-MGMT^{P140K} expression cassette allowing for in vivo selection of transduced cells with the latter two cassettes flanked by FRT and transposon sites.

Particular embodiments describe CRISPR/Cas9-mediated genome editing approaches in adult CD34+ cells aimed toward the reactivation of fetal γ-globin expression in red blood cells. Because models involving erythroid differentiation of CD34+ cells have limitations in assessing γ-globin reactivation, human β-globin locus-transgenic, a helper-dependent human CD46-targeting adenovirus vector expressing CRISPR/Cas9 (HDAd-HBG-CRISPR) was used to disrupt a repressor binding region within the γ-globin promoter.

Particular embodiments provide an integrating CD46 targeted Ad35 vector system: transgene included (i) a β-globin locus control region (LCR) driving expression of a γ globin gene, and (ii) EF1-α (constitutive promoter) driving expression of a MGMT^{P140K} cassette for positive selection of in vivo gene-modified HSC.

Particular embodiments provide an integrating CD46 targeted Ad35 vector system: transgene included (i) a 21.5 kb (long) human β-globin locus control region (LCR (HS1-HS5)) and a β-globin promoter (1.6 kb), driving expression of a γ globin gene (optionally including its 3′ UTR), and (ii) EF1-α (constitutive promoter) driving expression of a MGMT^{P140K} cassette for positive selection of in vivo gene-modified HSC. Some embodiments can further include a 3′HS1 (human β-globin 3′HS1; 3 kb, e.g., where 3′HS1 has the sequence of positions 5206867-5203839 of chromosome 11). In various embodiments, a 3′HS1 has the following nucleic acid sequence as shown in SEQ ID NO: 287, or a sequence having at least 80% sequence identity to SEQ ID NO: 287, e.g., a sequence having at least 80%, 85%, 90%, 91%, 92%, 93%, 94%, 95%, 96%, 97%, 98%, or 99% A identity to SEQ ID NO: 287. These embodiments can utilize a hyperactive transposase (e.g., SB100X) in combination with a recombinase system (e.g., Flp/Frt; Cre/Lox). Thus, in one particular embodiment, an Ad35 vector system can include, e.g., a transposable transgene insert including a long human β-globin locus control region (21.5 kb), a human β-globin promoter (1.6 kb), a human γ globin gene together with its 3′ UTR (2.7 kb), a human β-globin 3′ UTR, and a 3′HS1 (3 kb). A transposable transgene insert can further include, e.g., EF1-α (constitutive promoter) driving expression of a MGMT^{P140K}. In various embodiments, an Ad35 vector system can include, e.g., a transposable transgene insert of 32.4 kb.

Particular embodiments provide miRNA regulation systems that are activated only when HSPCs are recruited to a tumor to control expression of therapeutic transgenes. These features of the disclosure are demonstrated with anti PDL1-γ1 as a transgene. These systems can be used to regulate expression of therapeutic transgene in the context of the tumor microenvironment.

In various embodiments, a microRNA control system can refer to a method or composition in which expression of a gene is regulated by the presence of microRNA sites (e.g., nucleic acid sequences with which a microRNA can interact), an example of which has been provided in Example 5. In particular embodiments, a microRNA control system regulated expression of a gene such that the gene is expressed exclusively in target cells, such as HSPCs e.g., tumor infiltrating HSPCs. In some embodiments, a nucleic acid (e.g., a therapeutic gene) encoding a protein or nucleic acid of interest (e.g., an anti-cancer agent such as a CAR, TCR, antibody, and/or checkpoint inhibitor, e.g., an αPD-L1 antibody (e.g., an αPD-L1γ1 antibody) that is a checkpoint inhibitor) includes, is associated with, or is operably linked with a microRNA site, a plurality of same microRNA sites, or a plurality of distinct microRNA sites. While those of skill in the art will be familiar with means and techniques of associating a microRNA site with a nucleic acid or portion thereof having a sequence that encodes a gene of interest, certain non-limiting examples are provided herein. For example, a gene of interest (e.g., a sequence encoding an αPD-L1γ1 antibody) can be present in a nucleic acid such that expression of the gene of interest is regulated by the presence of one or more microRNA sites that suppress expression in cells that are not tumor-infiltrating leukocyte cells, but do not suppressed expression in tumor-infiltrating leukocytes. In certain particular examples, a gene of interest (e.g., a sequence encoding an αPD-L1γ1 antibody) can be present in a nucleic acid such that expression of the gene of interest is regulated by the presence of one or more miR423-5p microRNA sites that suppress expression in cells that are not tumor-infiltrating leukocyte cells, but do not suppressed expression in tumor-infiltrating leukocytes. In various embodiments, a microRNA control system can include a nucleic acid that includes, or in which expression of a protein or nucleic acid of interest is regulated by, one or more microRNA sites, e.g., 1, 2, 3, 4, 5, 6, 7, 8, 9, 10, or more microRNA sites. In various embodiments, a microRNA control system can include a nucleic acid that includes, or in which expression of a protein or nucleic acid of interest is regulated by, one or more miR423-5p microRNA sites, e.g., 1, 2, 3, 4, 5, 6, 7, 8, 9, 10, or more miR423-5p microRNA sites. In some particular embodiments, a microRNA control system can include a nucleic acid that encodes αPD-L1γ1 antibody and includes, or in which expression of αPD-L1γ1 antibody is regulated by, one or more miR423-5p microRNA sites, e.g., 1, 2, 3, 4, 5, 6, 7, 8, 9, 10, or more miR423-5p microRNA sites, e.g., miR423-5p microRNA sites.

The current disclosure describes recombinant Ad35 vectors targeting CD46 for in vivo gene editing of hematopoietic stem cells and related gene therapy improvements. In particular embodiments, the Ad35 delivery vector has all viral protein encoding genes removed and replaced with components associated with a therapeutic use. Removal of all genes encoding viral proteins provides a vector carrying capacity of 30 kb, significantly more space than is available with other viral vector delivery platforms. In particular embodiments, the Ad35 vector is helper-dependent, and the current disclosure also provides newly-designed Ad35 helper vectors. For the avoidance of doubt, the term “gene editing” as used herein includes, without limitation, any use of a vector or agent to modify a nucleic acid sequence.

Further provided herein are vectors that are or include nucleic acids provided herein, including without limitation microRNA control systems and other nucleic acids including microRNA (also referred to herein as miRNA) sites (also referred to herein as target sites) disclosed herein, and/or encode an agent disclosed herein, including without limitation an antibody such as an αPD-L1 antibody (e.g., an αPD-L1γ1 antibody). In any of the various embodiments of the present disclosure, a vector can be an Ad5/35 vector, optionally wherein the Ad5/35 vector is a helper-dependent Ad5/35 (HDAd5/35). In any of the various embodiments of the present disclosure, a vector can be an Ad5/35 vector (e.g., HDAd5/35 vector) including variations (e.g., amino acid mutations) provided herein, certain of which such vectors can be designated as Ad5/35++ (e.g., HDAd5/35++). For the avoidance of doubt, it is intended that those of skill in the art appreciate from the present disclosure that any embodiment using any vector, including embodiments in which a vector other than an Ad5/35 (e.g., other than Ad5/35++ or other than HDAd5/35++) vector is specified, is to be specifically read as disclosing, in addition to such vectors as stated in the relevant text, a vector that is an Ad5/35 vector (including, e.g., any of HDAd5/35, Ad5/35++, and HDAd5/35++ vector).

In any of the various embodiments of the present disclosure, a vector can be an Ad35 vector, optionally wherein the Ad35 vector is a HDAd35. In any of the various embodiments of the present disclosure, a vector can be an Ad35 vector (e.g., HDAd35 vector) including variations (e.g., amino acid mutations) provided herein, certain of which such vectors can be designated as Ad35++ (e.g., HDAd35++). For the avoidance of doubt, it is intended that those of skill in the art appreciate from the present disclosure that any embodiment using any vector, including embodiments in which a vector other than an Ad35 (e.g., other than Ad35++ or other than HDAd35++) vector is specified, is to be specifically read as disclosing, in addition to such vectors as stated in the relevant text, a vector that is an Ad35 vector (including, e.g., any of HDAd35, Ad35++, and HDAd35++ vector).

As indicated, the vectors described herein have many uses including in the treatment of sickle cell disease, γ globin gene addition and reactivation, and the targeting of multiple target sites for γ globin reactivation. Further, in addition to factor VIII (FVIII), the application of disclosed approaches can be used for other secreted proteins, including for example: (i) other coagulation factors, specifically FXI, FVII, von Willebrand factor (VWF), and rare clotting factors (i.e. factors I, II, V, X, XI, or XIII); (ii) enzymes that are currently used for Enzyme replacement therapies (ERT) for lysosomal storage diseases (taking advantage of the cross-correction mechanism) like Pompe disease (acid alpha (α)-glucosidase), Gaucher disease (glucocerebrosidase), Fabry disease (α-galactosidase A), and Mucopolysaccharidosis type I (α-L-Iduronidase); (iii) immunodeficiencies (e.g. SCID-ADA (adenosine deaminase)); (iv) cardiovascular diseases, e.g. familial apolipoprotein E deficiency and atherosclerosis (ApoE); (v) viral infections by expression of viral decoy receptors (e.g. for HIV-soluble CD4, or broadly neutralizing antibodies (bNAbs)) for HIV, chronic HCV, or HBV infections; (vi) cancer (e.g. controlled expression of monoclonal antibodies (e.g. trastuzumab) or checkpoint inhibitors (e.g. αPDL1) or protection of HSCs in order to permit therapeutic doses of chemotherapy and (vii) FANCA genes for Fanconi anemia; (viii) a coagulation factor deficiency optionally selected from hemophilia A, hemophilia B, or Von Willebrand Disease, (ix) a platelet disorder, (x) anemia, (xi) alpha-1 antitrypsin deficiency, or (xii) an immune deficiency. Other additional uses are described in more detail elsewhere herein.

Thus, one embodiment provides a recombinant serotype 35 adenovirus (Ad35) vector targeting CD46 for in vivo gene editing of hematopoietic stem cells.

Another embodiment is an erythrocyte genetically modified to express a therapeutic protein. By way of example, the therapeutic protein in some cases includes a coagulation factor or a protein that blocks or reduces viral infection. Optionally, the erythrocyte secretes the therapeutic protein.

Also provided are uses of the recombinant Ad35 vectors or erythrocytes described herein. These uses include to increase HbF reactivation by simultaneously targeting the erythroid bcl11a-enhancer and the HBG promoter regions; fora combination of γ-globin gene addition and endogenous γ-globin gene reactivation; for in vivo CRISPR genome engineering; to provide a therapeutic gene; to treat a (i) hemoglobinopathy, (ii) Fanconi anemia, (iii) a coagulation factor deficiency optionally selected from hemophilia A, hemophilia B, or Von Willebrand Disease, (iv) a platelet disorder, (v) anemia, (vi) alpha-1 antitrypsin deficiency, or (v) an immune deficiency; to treat thalassemia; to treat cancer, prevent or delay cancer recurrence or prevent or delay cancer onset in carriers of high-risk germ-line mutations, optionally wherein the cancer is breast cancer or ovarian cancer; for self-inactivation of CRISPR/Cas9; and for targeted integration using HDAd as donor vectors with a self-releasing cassette. Any of these uses may optionally include mobilization, for instance wherein the mobilization includes administration of Gro-beta, GM-CSF, S-CSF, and/or AMD3100.

Yet another use embodiment is use of any of the recombinant Ad35 vectors or erythrocytes described herein which includes administering a steroid (e.g., a glucocorticoid or dexamethasone), an IL-6 receptor antagonist, and/or an IL-1R receptor antagonist to a subject receiving the Ad35 vector and/or erythrocyte.

Also provided are use embodiments employing any of the recombinant Ad35 vectors or erythrocytes described herein, which include administering O⁶BG and TMZ (temozolomide) or BCNU (Carmustine) to a subject receiving the Ad35 vector and/or erythrocyte. By examples of such uses embodiments, the subject in is receiving O⁶BG and TMZ or BCNU as a treatment for anaplastic astrocytoma, breast cancer, colorectal cancer, diffuse intrinsic brainstem glioma, Ewing sarcoma, glioblastoma multiforme (GBM), malignant glioma, melanoma, metastatic malignant melanoma, nasopharyngeal cancer, or a pediatric cancer.

Yet another embodiment is a recombinant adenoviral serotype 35 (Ad35) vector production system including: a recombinant Ad35 helper genome including: a nucleic acid sequence encoding an Ad35 fiber shaft; a nucleic acid sequence encoding an Ad35 fiber knob; and recombinase DRs flanking at least a portion of an Ad35 packaging sequence, and a recombinant helper dependent Ad35 donor genome including: a 5′ Ad35 ITR; a 3′ Ad35 ITR; an Ad35 packaging sequence; and a nucleic acid sequence encoding at least one heterologous expression product.

Also provided are recombinant adenoviral serotype 35 (Ad35) helper vector embodiments that include: an Ad35 fiber shaft; an Ad35 fiber knob; and an Ad35 genome including recombinase DRs flanking at least a portion of an Ad35 packaging sequence.

Also provided are recombinant Ad35 helper genome embodiments that include: a nucleic acid sequence encoding an Ad35 fiber shaft; a nucleic acid sequence encoding an Ad35 fiber knob; and recombinase DRs flanking at least a portion of an Ad35 packaging sequence.

Also provided are recombinant helper dependent Ad35 donor vector embodiments that include: a nucleic acid sequence including: a 5′ Ad35 ITR; a 3′ Ad35 ITR; an Ad35 packaging sequence; and a nucleic acid sequence encoding at least one heterologous expression product, wherein the genome does not include a nucleic acid sequence encoding an Ad35 viral structural protein; and an Ad35 fiber shaft and/or an Ad35 fiber knob.

Also provided are recombinant helper dependent Ad35 donor genome embodiments that include: a 5′ Ad35 ITR; a 3′ Ad35 ITR; an Ad35 packaging sequence; and a nucleic acid sequence encoding at least one heterologous expression product, wherein the Ad35 donor genome does not include a nucleic acid sequence encoding an expression product encoded by the wild-type Ad35 genome.

Another embodiment is a method of producing a recombinant helper dependent Ad35 donor vector, the method including isolating the recombinant helper dependent Ad35 donor vector from a culture of cells, wherein the cells include: a recombinant Ad35 helper genome including: a nucleic acid sequence encoding an Ad35 fiber shaft; a nucleic acid sequence encoding an Ad35 fiber knob; and recombinase DRs flanking at least a portion of an Ad35 packaging sequence, and a recombinant helper dependent Ad35 donor genome including: a 5′ Ad35 ITR; a 3′ Ad35 ITR; an Ad35 packaging sequence; and a nucleic acid sequence encoding at least one heterologous expression product.

Also provided are recombinant Ad35 production system embodiments including: a recombinant Ad35 helper genome including: a nucleic acid sequence encoding an Ad35 fiber shaft; a nucleic acid sequence encoding an Ad35 fiber knob; and recombinase DRs within 550 nucleotides of the 5′ end of the Ad35 genome that functionally disrupt the Ad35 packaging signal but not the 5′ Ad35 ITR, and a recombinant Ad35 donor genome including: a 5′ Ad35 ITR; a 3′ Ad35 ITR; an Ad35 packaging sequence; and a nucleic acid sequence encoding at least one heterologous expression product.

Another embodiment is a recombinant Ad35 helper vector including: an Ad35 fiber shaft; an Ad35 fiber knob; and an Ad35 genome including recombinase DRs within 550 nucleotides of the 5′ end of the Ad35 genome that functionally disrupt the Ad35 packaging signal but not the 5′ Ad35 ITR.

Another embodiment is a recombinant Ad35 helper genome including: a nucleic acid sequence encoding an Ad35 fiber shaft; a nucleic acid sequence encoding an Ad35 fiber knob; and DRs within 550 nucleotides of the 5′ end of the Ad35 genome that functionally disrupt the Ad35 packaging signal but not the 5′ Ad35 ITR.

Another embodiment is a method of producing a recombinant helper dependent Ad35 donor vector, the method including isolating the recombinant helper dependent Ad35 donor vector from a culture of cells, wherein the cells include: a recombinant Ad35 helper genome including: a nucleic acid sequence encoding an Ad35 fiber shaft; a nucleic acid sequence encoding an Ad35 fiber knob; and recombinase DRs within 550 nucleotides of the 5′ end of the Ad35 genome that functionally disrupt the Ad35 packaging signal but not the 5′ Ad35 ITR, and a recombinant Ad35 donor genome including: a 5′ Ad35 ITR; a 3′ Ad35 ITR; an Ad35 packaging sequence; and a nucleic acid sequence encoding at least one heterologous expression product.

Yet another embodiment is a cell including a helper vector, a helper genome, a donor vector, or a donor genome as described herein, optionally wherein the cell is a HEK293 cell.

Another embodiment is a cell including a donor genome of any one of embodiments described herein, optionally wherein the cell is an erythrocyte, optionally wherein the cell is a hematopoietic stem cell, T-cell, B-cell, or myeloid cell, optionally wherein the cell secretes the expression product.

Also provided is a method of modifying a cell, the method including contacting the cell with an Ad35 donor vector according to any one of the provided Ad35 donor vector embodiments.

Also provided is a method of modifying a cell of a subject, the method including administering to the subject an Ad35 donor vector according to any one of the Ad35 donor vector embodiments, optionally wherein the method does not include isolation of the cell from the subject.

Yet another embodiment is a method of treating a disease or condition in a subject in need thereof, the method including administering to the subject an Ad35 donor vector according to any one of the Ad35 donor vector embodiments provided herein, optionally wherein the administration is intravenous.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Adoptive cell therapy: As used herein, “adoptive cell therapy” or “ACT” involves transfer of cells with a therapeutic activity into a subject, e.g., a subject in need of treatment for a condition, disorder, or disease. In some embodiments, ACT includes transfer into a subject of cells after ex vivo and/or in vitro engineering and/or expansion of the cells.

Affinity: As used herein, “affinity” refers to the strength of the sum total of non-covalent interactions between a particular binding agent (e.g., a viral vector), and/or a binding moiety thereof, with a binding target (e.g., a cell). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of k_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent. As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Allogeneic: As used herein, term “allogeneic” refers to any material derived from one subject which is then introduced to another subject, e.g., allogeneic T cell transplantation.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Chimeric antigen receptor. As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen. A T cell that has been genetically engineered to express a chimeric antigen receptor may be referred to as a CAR T cell. Thus, for example, when certain CARs are expressed by a T cell, binding of the CAR extracellular binding moiety with a target antigen can activate the T cell. CARs are also known as chimeric T cell receptors or chimeric immunoreceptors.

Combination therapy: As used herein, the term “combination therapy” refers to administration to a subject of to two or more agents or regimens such that the two or more agents or regimens together treat a condition, disorder, or disease of the subject. In some embodiments, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term “downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a formulation necessary to result in a desired physiological change in a subject. Effective amounts are often administered for research purposes.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% A or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Promoter. As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N.Y., 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, ora plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

### BRIEF DESCRIPTION OF THE FIGURES

Many of the drawings submitted herein are better understood in color. Applicant considers the color versions of the drawings as part of the original submission and reserve the right to present color images of the drawings in later proceedings.

FIG. 1. Exemplary vector schematics. The exemplary vector schematics show possible arrangements of components in integrated cassettes and transient expression cassettes useful in embodiments of the provided Ad35 vectors. The integrated cassettes include a transposon and other components between the frt sites. HDAd vectors can include expression products (Exp. Product) such as γ-globin, GFP, mCherry, and hFVIII(ET3); promoter(s) such as EF1α, PGK promoter, or the β promoter; selection marker(s) such as mgmt^{P140K}; regulatory elements (Reg. Elements) such as promoters, polyA tails, and/or insulators (such as cHS4). Transient expression cassettes include similar components, as well as DNA Cutting Molecule(s) (e.g., spCas9) or base editor(s) and genome targeting guide (GTG; e.g. sgRNA). Transposase vectors include a targeted recombinase (e.g., FIpE) and a transposase (e.g., SB100x). The vectors, although illustrated in one orientation/direction, can alternatively be provided in the reverse direction.

FIGS. 2A-2F. Integrating HDAd5/35++ vector for HSPC gene therapy of hemoglobinopathies. (FIG. 2A) Vector structure. In HDAd-γ-globin/mgmt, the 11.8-kb transposon is flanked by inverted transposon repeats (IR) and FRT sites for integration through a hyperactive Sleeping Beauty transposase (SB100X) provided from the HDAd-SB vector (right panel). The γ-globin expression cassette contains a 4.3-kb version of the β-globin LCR including 4 DNase hypersensitivity (HS) regions and the 0.7-kb β-globin promoter. The 76-Ile HBG1 gene including the 3′-UTR (for mRNA stabilization in erythrocytes) was used. To avoid interference between the LCR/β-promoter and EF1A promoter, a 1.2-kb chicken HS4 chromatin insulator (Ins) was inserted between the cassettes. The HDAd-SB vector contains the gene for the activity-enhanced SB100X transposase and Flpe recombinase under the control of the ubiquitously active PGK and EFTA promoters, respectively. (FIG. 2B) In vivo transduction of mobilized CD46tg mice. HSPCs were mobilized by s.c. injections of human recombinant G-CSF for 4 days followed by 1 s.c. injection of AMD3100. Thirty and 60 minutes after AMD3100 injection, animals were injected i.v. with a 1:1 mixture of HDAd-γ-globin/mgmt plus HDAd-SB (2 injections, each 4×10¹⁰ viral particles). Mice were treated with immunosuppressive (IS) drugs for the next 4 weeks to avoid immune responses against the human γ-globin and MGMT^{P140K}. O⁶-BG/BCNU treatment was started at week 4 and repeated every 2 weeks 3 times. With each cycle the BCNU concentration was increased, from 5 to 7.5 to 10 mg/kg. Immunosuppression was resumed 2 weeks after the last O⁶-BG/BCNU injection. (FIG. 2C) Percentage of human γ-globin⁺ peripheral RBCs measured by flow cytometry. (FIG. 2D) Percentage of human γ-globin⁺ cells in peripheral blood mononuclear cells (MNC), total cells, erythroid Ter119⁺ cells, and nonerythroid Ter119⁻ cells. (FIG. 2E) Percentage of human γ-globin protein compared with adult mouse globin chains (α, β-major, β-minor) measured by HPLC in RBCs at week 18. (FIG. 2F) Percentage of human γ-globin mRNA compared with adult mouse β-major globin mRNA measured by RT-qPCR in total in peripheral blood cells at week 18. Mice that did not receive any treatment were used as a control. In FIGS. 2C-2F, each symbol represents an individual animal.

FIG. 3. HPLC analysis of globin chains in RBCs from a hCD46tg control mouse and a representative CD46tg mouse after in vivo transduction/selection. The numbers (Volts) indicate the peak intensities. A total of 4 mice from each group was analyzed with similar results. The data are summarized in FIG. 2E. In FIG. 3, area under the curve (AUC) values are offset to the left of the corresponding peak.

FIGS. 4A-4C. Analysis of mice that received transplantations with bone marrow Lin− cells harvested at week 18 after in vivo transduction (“secondary recipients”). (FIG. 4A) Engraftment measured in blood samples at the indicated time points based on the percentage of human CD46-positive cells in PBMCs. (FIG. 4B) Engraftment in bone marrow, spleen, and PBMCs at week 20. (FIG. 4C) Ratio of human γ- to mouse α-globin protein measured by HPLC in RBCs. Each symbol represents an individual animal. Statistical analyses were done with the non-parametric Kruskal-Wallis test.

FIGS. 5A-5E. Analysis of transgene integration in bone marrow cells of week 20 secondary recipients. (FIG. 5A) Localization of integration sites on mouse chromosomes of bone marrow cells. Shown is a representative mouse. Each line is an integration site. The number of integration sites in this sample is 2,197. (FIG. 5B) Distribution of integrations in genomic regions. Integration site data from 5 mice were pooled and used to generate the graph. (FIG. 5C) The number of integrations overlapping with continuous genomic windows and randomized mouse genomic windows and size was compared. Pooled data were used as in FIG. 5B). The Pearson's χ² test P value for similarity is 0.06381, implying that the integration pattern is close to random. (FIG. 5D) Transgene copy numbers. Genomic DNA from total bone marrow cells from untransduced control mice and week 20 secondary recipients was subjected to qPCR with human γ-globing-specific primers. Shown is the copy number per cell for individual animals. Each symbol represents an individual animal. (FIG. 5E) Transgene copy numbers in individual clonal progenitor colonies. Bone marrow Lin⁻ cells were plated in methylcellulose, and individual colonies were picked 15 days later. qPCR was performed on genomic DNA. Shown is normalized qPCR signal in individual colonies expressed as transgene copy number per cell (n=113). Each symbol represents the copy number in an individual colony derived from a single cell.

FIG. 6. qPCR in single cell-derived progenitor colonies to measure the VCN (see FIG. 7E).

FIGS. 7A-7E. Hematological parameter after in vivo HSPC transduction/selection in CD46tg mice (week 18 after HDAd injection). (FIG. 7A) WBC counts. (FIG. 7B) Representative blood smears from an untreated mouse and a mouse at week 18 after HDAd-γ-globin/mgmt plus HDAd-SB injection. Scale bar: 20 μm. Nuclei of WBCs stain purple. (FIG. 7C) Hematological parameters. Hb, hemoglobin; HCT, hematocrit; MCV, mean corpuscular volume; MCH, mean corpuscular hemoglobin; MCHC, mean corpuscular hemoglobin concentration; RDW, red cell distribution width. n 3, *P<0.05. Statistical analysis was performed using 2-way ANOVA. (FIG. 7D) Cellular bone marrow composition in naive mice (control) and treated mice sacrificed at week 18. Shown is the percentage of lineage marker-positive cells (Ter119+, CD3+, CD19+, and Gr-1+ cells) and HSPCs (LSK cells). (FIG. 7E) Colony-forming potential of bone marrow Lin− cells harvested at week 18 after in vivo transduction. Shown is the number of colonies that formed after plating of 2,500 Lin− cells. In FIG. 7A and FIGS. 7C-7E, each symbol represents an individual animal. NE, neutrophils; LY, lymphocytes; MO, monocytes; BA, basophils.

FIG. 8. Generation of the CD46++/Bhhth-3 thalassemic model. Female CD46tg mice were bred with male Hbbth-3 mice. The F1 hybrid mice were back-crossed with hCD46+/+ mice to generate Hbbth-3 mice homozygous for hCD46+/+

FIGS. 9A-9C. Phenotype of the CD46+/+/Hbbth-3 mouse thalassemia model. (FIG. 9A) Hematological parameters of CD46+/+/Hbbth-3 mice (n=7) as compared with CD46tg (n=3) and Hbbth-3 mice (n=3). Each symbol represents an individual animal. *P≤0.05, **P≤0.0002, ***P≤0.00003. Statistical analysis was performed using 2-way ANOVA. RET, reticulocytes. (FIG. 9B) Representative peripheral blood smears after staining with May-Grunwald/Giemsa. Scale bar: 20 μm. (FIG. 9C) Extramedullary hemopoiesis by H&E staining in liver and spleen sections of CD46^{+/+}/Hbbth-3 mice (bottom left 2 panels) as compared with spleen and liver sections of CD46tg mice (top left 2 panels). Scale bars: 20 μm. Clusters of erythroblasts in the liver are indicated in the bottom left panel. Circles in the bottom middle panel mark megakaryocytes in the spleen. Iron deposition (granular bluish deposits) by Perl's Prussian Blue staining in the spleen are shown in the top right panel for CD46tg and the bottom right panel for CD46^{+/+}/Hbbth-3 mice. Scale bar: 25 μm.

FIG. 10. Analysis of white blood cells in thalassemic mice (Hbbth-3 and CD46^{+/+}/Hbbth-3) compared to “healthy” CD46tg mice. WBCs: white blood cells, NEU: neutrophils, LY: lymphocytes, MONO: monocytes. **p≤0.05, **p≤0.0002, ***p≤00003. These are baseline levels in mice before treatment. (n=8 for CD46tg, n=4 for Hbbth3, n=20 for CD46++/Hbbth3). Each symbol represents an individual animal. Statistical analyses were done with the non-parametric Kruskal-Wallis test.

FIG. 11. Mobilization of HSPCs in CD46^{+/+}/Hbbth-3 mice. Shown are the numbers of mobilized LSK (Lineage-/Sca-1+/c-Kit+/) cells in peripheral blood at 1 hour after the last AMD3100 injection. n=17 mobilized mice; n=3 untreated mice. Statistical analyses were done with the non-parametric Kruskal-Wallis test.

FIG. 12. In vivo transduction/selection of mobilized CD46^{+/+}/Hbbth-3 mice. In vivo transduction of mobilized CD46^{+/+}/Hbbth3 mice. HSPCs were mobilized by s.c. injections of human recombinant G-CSF for 6 days (days 1-6) followed by three s.c. injections of AMD3100/Plerixafor (days 5-7). 30 and 60 minutes after Plerixafor injection, animals were intravenously injected with a 1:1 mixture of HDAd-γ-globin/mgtm+HDAd-SB (2 injections, each 4×10¹⁰ vp). Following in vivo transduction, immuno-suppression was administered for 17 weeks to avoid immune responses against the human γ-globin and MGMT^{P140K} proteins. At week 17, treated mice either served as donors for secondary transplants or were subjected to in vivo selection with O⁶-BG/BCNU. Secondary C57Bl/6 recipients were followed for 16 weeks under immunosuppression and then sacrificed. Mice subjected to in vivo selection received an escalating (5, 7.5, 10, 10 mg/kg) O⁶-BG/BCNU treatment every other week. Immuno-suppression was resumed two weeks after the last O⁶-BG/BCNU dose. At week 29, mice were sacrificed, and their bone marrow was transplanted into C57Bl/6 secondary recipients.

FIGS. 13A-13F. Analysis of in vivo-transduced CD46^{+/+}/Hbbth-3 mice that did not receive O⁶BG/BCNU treatment. (FIG. 13A) Percentage of human γ-globin in peripheral RBCs measured by flow cytometry. The experiment was performed 3 times, indicated by different symbol shapes. (FIG. 13B) γ-Globin expression in erythroid (Ter119⁺) and nonerythroid (Ter119⁻) blood cells. ***P≤0.00003 by 1-way ANOVA test. (FIG. 13C) RBC analysis of healthy (CD46tg) mice (n=3), CD46^{+/+}/Hbbth-3 mice prior to mobilization and in vivo transduction (n=14), and CD46^{+/+}/Hbbth-3 mice that underwent in vivo transduction and were analyzed at week 16 (n=8). *P≤0.05. Statistical analysis was performed using 2-way ANOVA. (FIG. 13D) Histological phenotype. Top: Blood smears. Middle: Supravital stain of peripheral blood smears with Brilliant cresyl blue for reticulocyte detection. The percentages of positively stained reticulocytes in representative smears were: for CD46tg, 8%±0.8%; for CD46^{+/+}/Hbbth-3 before transduction, 39%±1.3%; and for CD46^{+/+}/Hbbth-3 week 16 after transduction, 26%±0.45%. Bottom: Extramedullary hemopoiesis. Scale bars: 20 μm. (FIG. 13E and FIG. 13F) Analysis of secondary recipients. Total bone marrow from week 16 in vivo-transduced mice was transplanted into C57BL/6 mice that received sublethal busulfan preconditioning. Mice received immunosuppression during the period of observation. (FIG. 13E) Engraftment based on the percentage of human CD46+ (hCD46+) PBMCs. (C57BL/6 recipients do not express hCD46.) (FIG. 13F) Percentage of human γ-globin⁺ RBCs. Each symbol represents an individual animal.

FIGS. 14A-14F. Analysis of γ-globin expression in in vivo-transduced CD46^{+/+}/Hbbth-3 mice after in vivo selection. (FIG. 14A) Percentage of human γ-globin in peripheral RBCs measured by flow cytometry. Arrows indicate the time points of O⁶-BG/BCNU treatment. Different symbols represent 3 independent experiments. The data up to week 16 are identical to those in FIG. 13A. (FIG. 14B) Percentage of γ-globin-expressing cells in hematopoietic tissues at sacrifice (week 29) analyzed by flow cytometry. *P≤0.05, **P≤0.0002, ***P≤0.00003. (FIG. 14C) γ-Globin expression in MACS-purified Ter119 cells. Bone marrow cells from primary recipients at week 29 were immunomagnetically selected for Ter119⁺ cells. γ-Globin expression was measured in Ter119⁺ and Ter119⁻ cells by flow cytometry. ***P≤0.0002. (FIG. 13D) Fold enrichment of γ-globin⁺ erythroid (Ter119+) and nonerythroid (Ter119⁻) cells in peripheral blood, bone marrow, and spleen before versus after in vivo selection (week 16 vs. week 29). n=5, **P≤0.0002. (FIG. 14E) Percentage of human γ-globin protein compared with mouse α-globin protein, measured by HPLC in RBCs. Statistical analyses were done with the nonparametric Kruskal-Wallis test. (FIG. 14F) Level of human γ-globin mRNA over adult mouse β-major globin mRNA measured by RT-qPCR in peripheral blood cells. Untreated CD46^{+/+}/Hbbth-3 mice were used as control. Each symbol represents an individual animal.

FIGS. 15A-15D. HPLC analysis of globin chains in RBCs. (FIG. 15A) Representative chromatograms of mouse globin peaks in a control CD46tg mouse. The peaks for adult mouse alpha (α), beta (β)-minor, and β-major globin are labeled. (FIGS. 15B-15D) Chromatogram of RBCs from a CD46^{+/+}/Hbbth-3 mice (#71). Note that these mice are heterozygous for β-minor and β-major gene deletions. The extra peaks around 29 min could be associated with this. In (FIG. 15D), the peak specific to human γ-globin is labeled. Representative chromatograms are shown. The numbers (Volts) indicate the peak intensities. In FIGS. 15C and 15D, AUC values are offset to the left of the corresponding peak.

FIG. 16. DNA analysis of treated CD46++/Hbbth-3 mice at week 29. Transgene (γ-globin) copy number per bone marrow cell. Each symbol represents an individual animal.

FIGS. 17A-17E. Phenotypic correction of CD46^{+/+}/Hbbth-3 mice by in vivo HSPC transduction/selection. (FIG. 17A) RBC analysis of healthy (CD46tg) mice, CD46^{+/+}/Hbbth-3 mice prior to mobilization and in vivo transduction, and CD46^{+/+}/Hbbth-3 mice that underwent in vivo transduction/selection (analyzed at week 29 after HDAd infusion) (n=5). *P≤0.05, **P≤0.0002, ***P≤0.00003. Statistical analysis was performed using 2-way ANOVA. (FIG. 17B) Supravital stain of peripheral blood smears with Brilliant cresyl blue for reticulocyte detection. Arrows indicate reticulocytes containing characteristic remnant RNA and micro-organelles. The percentages of positively stained reticulocytes in representative smears were: for CD46, 7%; for CD46^{+/+}/Hbbth-3 before treatment, 31%; and for CD46^{+/+}/Hbbth-3 after treatment, 12%. Scale bar: 20 μm. (FIG. 17C) Top: Blood smears. Scale bar: 20 μm. Middle: Bone marrow cytospins. Arrows indicate erythroblasts at different stages of maturation and a backshift in erythropoiesis with pro-erythroblast predominance in treated mice. Scale bar: 25 μm. Bottom: Tissue hemosiderosis by Perl's stain. Iron deposition is shown as cytoplasmic blue pigments of hemosiderin in spleen tissue sections. The blood smear images for the control mice (CD46tg and CD46^{+/+}/Hbbth-3, before transduction) in (FIG. 17C) and (FIG. 18D) are from the same sample. (FIG. 17D) Macroscopic spleen images of 1 representative CD46tg and 1 untreated CD46^{+/+}/Hbbth-3 mouse and 5 treated CD46^{+/+}/Hbbth-3 mice. (FIG. 17E) At sacrifice, spleen size was determined as the ratio of spleen weight to total body weight (mg/g). Each symbol represents an individual animal. Data are presented as means±SEM. *P≤0.05. Statistical analysis was performed using 1-way ANOVA.

FIGS. 18A-18E. Analysis of secondary C57BL/6 recipients with transplanted bone marrow cells from treated CD46^{+/+}/Hbbth-3 mice. (FIG. 18A) Engraftment rates measured in the periphery based on the percentage of human CD46+ (hCD46+) cells in PBMCs after busulfan conditioning or total-body irradiation (TBI). (C57BL/6 recipients do not express hCD46.) (FIG. 18B) Percentage of human γ-globin-expressing peripheral blood RBCs. All mice received immunosuppression starting from week 4 after transplantation. (FIG. 18C) Percentage of γ-globin⁺ cells in hCD46+ (donor-derived) cells. (FIG. 18C and FIG. 18D) γ-Globin/CD46 expression in secondary C57BL/6 recipients at week 20 after transplant (busulfan preconditioning). CD46+ cells were immunomagnetically separated from the chimeric bone marrow of 3 representative secondary mice and analyzed for γ-globin expression by flow cytometry. Notably, unlike humans, huCD46tg mice express CD46 on RBCs. (FIG. 18C) γ-Globin/CD46 marking rates of primary and secondary recipients at sacrifice. (FIG. 18D) γ-Globin expression in CD46+-selected cells from the hematopoietic tissues of secondary recipients (week 20). Each symbol represents an individual animal. (FIG. 18E) γ-Globin expression in secondary recipients that received a new (second) round of HSPC mobilization/in vivo transduction (n=5). Secondary recipients (busulfan-preconditioned) were analyzed for γ-globin and CD46 expression at week 20 after transplantation (“Before in vivo transduction”). These mice were then mobilized and transduced in vivo with the HDAd-γ-globin plus HDAd-SB vectors. Four weeks after in vivo transduction, mice were sacrificed and analyzed (“Week 4 after in vivo transduction”). ***P≤0.00003. Statistical analyses were performed using 1-way ANOVA.

FIGS. 19A-19D. Safety of in vivo transduction/selection in the CD46^{+/+}/Hbbth-3 mouse model. (FIG. 19A) WBC and platelet (PLT) counts during and after in vivo selection. O⁶BG/BCNU treatment is indicated by asterisks. n≥3. (FIG. 19B) Absolute numbers of circulating WBC subpopulations. n 3. (FIG. 19C) Cellular bone marrow composition in control and treated mice sacrificed at week 29. Shown is the percentage of lineage marker-positive cells (Ter119+, CD3+, CD19+, and Gr-1+ cells) and HSPCs (LSK cells). (FIG. 19D) Colony-forming potential of bone marrow cells harvested at week 29. Each symbol represents an individual animal. *P≤0.05, **P≤0.0002, ***P≤0.00003. Statistical analyses were performed using 2-way ANOVA. NEU: neutrophils; LY: lymphocytes; MO: monocytes.

FIGS. 20A-20F. Effect of anti-HDAd5/35⁺⁺ antibodies on a second round of transduction. (FIG. 20A) CD46tg mice were mobilized and injected with HDAd-mgmt/GFP+HDAd-SB. Serum samples were collected as indicated. (FIG. 20B, FIG. 20C) Flow cytometry analysis of PBMCs at day 4 and week 4 after mobilization/transduction. (FIG. 20D) Second round of mobilization/transduction at week 4 and subsequent GFP analysis. (FIG. 20E) anti-HDAd5/35⁺⁺ antibody titers based on OD₄₅₀. An OD₄₅₀=0.2 titer is considered to be neutralizing. (FIG. 20F) Percentage of GFP-positive PBMCs measured in different cohorts (see FIGS. 20B-20D). Ctrl are untreated CD46tg mice. Each symbol in (FIG. 20E) and (FIG. 20F) represents an individual animal. Statistical analyses were done with the non-parametric Kruskal-Wallis test.

FIGS. 21A-21D. Vector DNA biodistribution at week 18 after HDAd injection (10 weeks in vivo selection) (FIG. 21A) Primer design. The light gray primers are specific to the transgene cassette and will detect both integrated and episomal vector DNA. The dark gray primers will detect vector stuffer DNA derived from plasmid pHCA. Upon SB100x-mediated integration, the corresponding target region for the dark gray primers will be lost. The dark gray primers are therefore used to measure episomal vector copies. (FIG. 21B) Standard curve of integrated transgene copy number. (FIG. 21C) Standard curve for HCA (episomal vector) copy number. (FIG. 21D) Integrated transgene copy number per cell. Episomal vector copies (dark gray primers) were subtracted from total vector copies (light gray primers). The vector-specific signals were normalized to GAPDH. Each symbol represents an individual animal.

FIGS. 22A-22C. In vitro assay to assess the mutagenicity of O⁶BG/BCNU treatment. (FIG. 22A) After overnight recovery from cryopreservation, CD34⁺ cells were transduced with HDAd-mgmt/GFP or HDAd control at an MOI of 3000 vp/cell which mediated GFP expression in 50% of cells two days later. Cells were then treated with 10 mM O⁶BG followed by 25 mM BCNU (or DMSO solvent) for 2 hours. After washing, cells were plated in methylcellulose for CFU assay (3000 cells per 35 mm dish). Colonies and pooled cells were counted 14 days later and genomic DNA subjected to whole exome sequencing. (FIG. 22B) Numbers of pooled cells per plate. Each symbol represents the cell number in an individual 35 mm dish. Statistical analyses were done with the non-parametric Kruskal-Wallis test. (FIG. 22C) Representative colony from the HDAd-mgmt/GFP+O⁶BG/BCNU group. It demonstrates GFP expression in the majority of cells with GFP fading at the colony periphery due to the loss of episomal viral genomes. The scale bar is 1 mm.

FIG. 23. Vector structures. HDAd-short-LCR: This vector contains a 4.3 kb mini-LCR consisting of the core regions of DNase hypersensitivity sites (HS) 1 to 4 and a 0.66 kb β-globin promoter. The length of the transposon is 11.8 kb. HDAd-long-LCR. The γ-globin gene is under the control of a 21.5 kb β-globin LCR (chr11: 5292319-5270789), a 1.6 kb β-globin promoter (chr11: 5228631-5227023 or chr11: 5228631-5227018, for instance) and a 3′HS1 region (chr11: 5206867-5203839) also derived from the β-globin locus. For RNA stabilization in erythroid cells, a γ-globin gene UTR was linked to the 3′ end of the γ-globin gene. The vector also contains an expression cassette for mgmtP140K allowing for in vivo selection of transduced HSPCs and HSPC progeny. The γ-globin and mgmt expression cassettes are separated by a chicken globin HS4 insulator (cHS4). The 32.4 kb LCR-γ-globin/mgmt transposon is flanked by inverted repeats (IRs) that are recognized by SB100x and by ftr sites that allow for the circularization of the transposon by Flpe recombinase. HDAd-SB: The second vector required for integration contains the expression cassettes for the activity-enhanced Sleeping Beauty SB100x transposase and the Flpe recombinase.

FIGS. 24A-24F. SB100x-mediated integration of the 32.4 kb transposon after ex vivo HSPC transduction study with HDAd-long-LCR. (FIG. 24A) Experimental regimen: Bone marrow Lin− cells from CD46-transgenic mice were transduced with HDAd-long-LCR and HDAd-SB at a total MOI of 500 vp/cell. After one day in culture, 1×106 transduced cells/mouse were transplanted into lethally irradiated C57Bl/6 mice. At week 4, O6BG/BCNU treatment was started and repeated four times every two weeks. With each cycle, the BCNU concentration was increased from 5 mg/kg, to 7.5 mg/kg, to 10 mg/kg (twice). At week 20, mice were sacrificed. (FIG. 24B) Percentage of human γ-globin-positive peripheral red blood cells (RBC) measured by flow cytometry. Each symbol is an individual animal. (FIG. 24C) Representative flow cytometry data showing human γ-globin-expression in erythroid (Ter119⁺) bone marrow cells (lower panel) at week 20 after transplantation. The top panel shows a mouse transplanted with mock-transduced cells. (FIG. 24D) Schematic of iPCR analysis: Five micrograms of genomic DNAs were digested with SacI, re-ligated, and subjected to nested, inverse PCR with the indicated primers (see Materials and Methods). (FIG. 24E) Agarose gel electrophoresis of cloned plasmids containing integration junctions. Indicated bands were excised and sequenced. The chromosomal localization of integration sites are shown below the gel. (FIG. 24F) Examples of junction sequences: 5′ end vector sequence, Sleeping beauty IR/DR sequence, integration junction (chr15, 6805206) SEQ ID NO: 1; 5′ end vector sequence, Sleeping beauty IR/DR sequence, integration junction (chrX, 16897322) SEQ ID NO: 2; 3′ end vector sequence, Sleeping beauty IR/DR sequence, integration junction (chr4, 10207667) SEQ ID NO: 3. The vector body and IR/DR sequences are designated in plain text and underlining, respectively. The chromosomal sequence is designated in bold text. The TA dinucleotides used by SB100x at the junction of the IR and chromosomal DNA are bracketed.

FIGS. 25A-25E. In vivo HSPC transduction with HDAd-long-LCR containing the 32.4 kb transposon and HDAd-short-LCR containing an 11.8 kb transposon. (FIG. 25A) Treatment regimen: hCD46tg mice were mobilized and IV injected with either HDAd-short-LCR+HDAd-SB or HDAd-long-LCR+HDAd-SB (2 times each 4×1010 vp of a 1:1 mixture of both viruses). Five weeks later, O6BG/BCNU treatment was started. With each cycle, the BCNU concentration was increased from 5 mg/kg, to 7.5 mg/kg, and 10 mg/kg. The O6BG concentration was 30 mg/kg in all four treatments. Mice were followed until week 20 when animals were sacrificed for analysis. Bone marrow Lin− cells were used for transplantation into secondary recipients. Secondary recipients were then followed for 16 weeks. (FIG. 25B) Percentage of human γ-globin-positive cells in peripheral red blood cells (RBCs) measured by flow cytometry. Each symbol is an individual animal. In mice that were mock-transduced, less than 0.1% of cells were γ-globin-positive. (FIG. 25C) γ-globin protein chain levels measured by HPLC in RBCs at week 20 after in vivo HSPC transduction. Shown are the percentages of human γ-globin to mouse α-globin protein chains. (FIG. 25D) γ-globin mRNA levels measured by qRT-PCR in total blood at week 20 after in vivo HSPC transduction. Shown are the percentages of human γ-globin mRNA to mouse α-globin mRNA. (FIG. 25E) Vector copy number per cell in bone marrow mononuclear cells, harvested at week 20 after in vivo HSPC transduction. The difference between the two groups is not significant. Statistical analyses were performed using two-way ANOVA.

FIGS. 26A-26D. Hematological parameters at week 20 after in vivo HSPC transduction. (FIG. 26A) White blood cells (WBC), neutrophils (NE), leukocytes (LY), monocytes (MO), eosinophils (EO), and basophils (BA). (FIG. 26B) Erythropoietic parameters. RBC: red blood cells, Hb: hemoglobin, MCV: mean corpuscular volume, MCH: mean corpuscular hemoglobin, MCHC: mean corpuscular hemoglobin concentration, RDW: red cell distribution width. The differences between the three groups were not significant. (FIG. 26C) Cellular bone marrow composition. (FIG. 26D) Colony-forming potential of bone marrow Lin⁻ cells. The differences between the groups were not significant in FIGS. 26A-26D.

FIG. 27. Schematic of insertion site analysis. The localization of NheI and KpnI sites in the HDAd-long-LCR vector in relation to the Sleeping Beauty inverted repeats (IRs) is indicated. These enzymes cut close, but outside of the SB IR/DR and are used to decrease the background of unintegrated vectors. Genomic DNA from bone marrow Lin− cells was digested with NheI and KpnI, and after heat inactivation, further digested with NIaIII. NIaIII is a 4-cutter and will create small DNA fragments. Digested DNA was then ligated with double stranded oligos with known sequence and compatible ends to the digested NIaIII fragments. Following heat-inactivation and clean-up, the linker-ligated products were used for linear amplification, which creates a single-stranded (ss) DNA population primed from the SB left arm. The primer is biotinylated, so the ssDNAs can be collected with streptavidin beads. After extensive washing, ssDNA was eluted from the beads and subjected to further amplification by two rounds of nested PCR. PCR amplicons were gel purified, cloned, sequenced and mapped to the mouse genome sequences to mark the integration sites.

FIGS. 28A-28D. Analysis of vector integration sites in HSPCs by LAM-PCR/NGS. Genomic DNA isolated from bone marrow cells harvested at week 20 after in vivo transduction with HDAd-long-LCR+HDAd-SB. (FIG. 28A) Chromosomal distribution of integration sites. The integration sites are marked by vertical lines. (FIG. 28B) Examples of junction sequences: Sleeping beauty IR/DR sequence, integration junction (chr7, 79796094) SEQ ID NO: 4; Sleeping beauty IR/DR sequence, Integration junction (repeat region) SEQ ID NO: 5. IR/DR sequences are designated by underlining and bold text. The chromosomal sequence is designated in plain text. The TA dinucleotides used by SB100x at the junction of the IR and chromosomal DNA are bolded. (FIG. 28C) Integration sites were mapped to the mouse genome and their location with respect to genes was analyzed. Shown is the percentage of integration events that occurred 1 kb upstream transcription start sites (TSS) (0.0%), 5′UTR of exons (0.0%), protein coding sequences (0.0%), introns (17.0%), 3′UTRs (0.0%), 1 kb downstream from 3′UTR (0.0%), and intergenic (83.0%). (FIG. 28D) Integration pattern in mouse genomic windows. The number of integrations overlapping with continuous genomic windows and randomized mouse genomic windows and size was compared. This shows that the pattern of integration is similar in continuous and random windows. Maximum number of integrations in any given window was not more than 3; with one integration per window having the higher incidence.

FIGS. 29A-29I. Analysis of secondary recipients. Bone marrow Lin− cells harvested at week 20 from in vivo transduced CD46tg mice were transplanted into lethally irradiated C57Bl/6 mice. Secondary recipients were followed for 16 weeks. (FIG. 29A) Engraftment rates based on the percentage of CD46-positive PBMCs at weeks 4, 8, 12, and 16 after transplantation. The differences between the two groups were not significant. (FIG. 29B) Percentage of γ-globin-expressing peripheral blood RBCs measured by flow cytometry. The differences between the two groups are not significant. (FIG. 29C) Vector copy number per cell in bone marrow MNCs harvested at week 20 after in vivo HSPC transduction. The difference between the two groups is not significant. (FIG. 29D) Analysis of human γ-globin chains by HPLC in RBCs of secondary recipients. Shown is the percentage of human γ-globin to adult mouse α-globin. ***p<0.0001. (FIG. 29E) γ-globin mRNA levels in total blood cells relative to mouse α-globin mRNA. (FIG. 29F) Percentage of γ-globin expressing erythroid (Ter119⁺ cells) in all bone marrow MNCs. Statistical analyses were performed using two-way ANOVA. (FIG. 29G) γ-globin mRNA levels bone marrow MNCs at week 16 p.t. Shown are percentages of human γ-globin m-RNA to mouse α and β-major globin mRNA. (FIG. 29H) Erythroid specificity. Percentage of γ-globin⁺ cells in erythroid (Ter119k) and non-erythroid (Ter119⁻) cells. (FIG. 29I) Vector copy number (VCN) per cell in bone marrow MNCs harvested at week 20 after in vivo HSPC transduction. The difference between the two groups is not significant.

FIGS. 30A-30D. Hematological parameters in secondary recipients at week 16 after transplantation. (FIG. 30A) White blood cells. (FIG. 30B) Erythropoietic parameters. RBC: red blood cells, Hb: hemoglobin, MCV: mean corpuscular volume, MCH: mean corpuscular hemoglobin, MCHC: mean corpuscular hemoglobin concentration, RDW: red cell distribution width. (FIG. 30C) Cellular bone marrow composition. (FIG. 30D) Colony-forming potential of bone marrow Lin− cells. The differences between the groups were not significant in FIGS. 30A-30D. Statistical analyses were performed using two-way ANOVA.

FIGS. 31A-31D. In vitro studies with human CD34+ cells. (FIG. 31A) Schematic of the experiment: CD34+ cells were transduced with HDAd-long-LCR+HD-SB or HDAd-short-LCR+HDAd-SB and subjected to erythroid differentiation (ED). In vitro selection with O6BG-BCNU was started at day 5 of ED. At day 18 cells were analyzed by flow cytometry (FIG. 31B) and HPLC (FIG. 31C). (FIG. 31D) Vector copy number at day 18. Statistical analyses were performed using two-way ANOVA. *p<0.05; **p<0.0001

FIGS. 32A-32H. Human γ-globin expression after in vivo HSC gene therapy of Hbb^{th3}/CD46 mice with HDAd-short-LCR and HDAd-long-LCR. (FIG. 32A) Treatment regimen. In contrast to FIGS. 25A-25E, FIGS. 32A-32D show results within thalassemic Hbb^{th3}/CD46 mice. (FIG. 32B) Percentage of human γ-globin-positive cells in peripheral red blood cells (RBCs) measured by flow cytometry. Each symbol is an individual animal. (FIG. 32C) γ-globin protein chain levels measured by HPLC in RBCs at week 18 after in vivo HSPC transduction. Shown are the percentages of human γ-globin to mouse α-globin protein chains. (FIG. 32D) Representative chromatograms of an untreated Hbb^{th3}/CD46 mouse (left panel) and a mouse at week 21 after treatment. Mouse α- and β-chains as well the added human γ-globin are indicated.

FIGS. 32E-32H. Human γ-globin expression after in vivo HSPC gene therapy of Hbbth3/CD46+/+ mice with HDAd-short-LCR and HDAd-long-LCR. (FIG. 32E) Treatment regimen: In contrast to the study shown in FIG. 25, this study was done with thalassemic Hbbth3/CD46 mice. (FIG. 32F) Percentage of human γ-globin-positive cells in peripheral red blood cells (RBCs) measured by flow cytometry. Each symbol is an individual animal. (FIG. 32G) γ-globin protein chain levels measured by HPLC in RBCs at weeks 10 to 16 after in vivo HSPC transduction. Shown are the percentages of human γ-globin to mouse α-globin protein chains. (FIG. 32H) Representative chromatograms of an untreated Hbbth3/CD46+/+mouse (left panel) and a mouse at week 16 after treatment. Mouse α- and β-chains as well the added human γ-globin are indicated. Notably, two independent studies were performed with Hbbth3/CD46+/+ mice. First study: N=6 for HD-long-LCR and N=2 for HDAd-short-LCR followed for 21 weeks. Second study: N=4 for HD-long-LCR and N=5 for HDAd-short-LCR followed for 16 weeks. FIG. 32F shows the combined data until week 21. Statistical analyses were performed using two-way ANOVA. *p<0.05; **p<0.0001

FIGS. 33A, 33B. Analysis of bone marrow at sacrifice. Bone marrow was harvested at week 16 after in vivo HSPC transduction of Hbbth3/CD46+/+ mice. (FIG. 33A) Vector copy number per cell in bone marrow MNCs. The difference between the two groups is not significant. (FIG. 33B) Mean Fluorescence Intensity (MFI) of γ-globin in erythroid (Ter119+) cells. Statistical analyses were performed using two-way ANOVA.

FIG. 34. Micrographs showing the normalized erythrocyte morphology of C57BL6 (Normal mice) and the Townes SCA mice, before treatment and at week 10 after treatment-long LCR.

FIG. 35. Micrographs showing the normalized erythropoiesis (reticulocyte count) for Townes mice, before treatment, and Townes mice at week 10, after treatment (long LCR).

FIGS. 36A-36C. Phenotypic correction. (FIGS. 36A, 36B) Blood cell morphology with left panel displaying blood smears stained with Giemsa stain and right panels displaying blood smears stained with May-Grunwald stain. Remnants of nuclei and cytoplasm in reticulocytes results in purple staining. (FIG. 36A) Comparison before and at week 14. (FIG. 36B) Comparison of Giemsa stain and reticulocytes for CD46tg, Hbb^{th3}/CD46 mice before, Hbb^{th3}/CD46 mice with HDAd-long-LCR at week 18, and Hbb^{th3}/CD46 mice with HDAd-long-LCR at week 21. (FIG. 36C) Bone marrow cytospins. Visible is a bac k-shift in erythropoiesis with pro-erythroblast predominance in treated. The scale bar is 20 μm.

FIGS. 37A, 37B. Phenotypic correction (week 16). (FIG. 37A) Left panels: Blood smears stained with Giemsa/May-Grunwald stain (5 min). Right panels: Blood smears stained with Brilliant cresyl blue for reticulocytes. Remnants of nuclei and cytoplasm in reticulocytes appear as purple staining. (FIG. 37B) Bone marrow cytospins stained with Giemsa/May-Grunwald stain (15 min). (FIGS. 37A and 37B) Upper panel: Normal bone marrow cellular distribution—erythroid lineage is represented by all stages of erythrocyte differentiation. Middle panel: Predominance of erythroid lineage over white cell lineage—erythroid lineage consists mainly of proerythroblasts and basophilic erythroblasts. Bottom panel: Normal bone marrow cellular distribution—erythroid lineage is mainly represented by maturing polychromatic and orthochromatic erythroblasts. The scale bars are 25 μm.

FIG. 38: Shows the graphical depiction for normalized erythrocyte parameters of Long LCR vectors, Short LCR vectors, and the control CD46tg, at Week 1 (top panel) and Week 10 (bottom panel).

FIGS. 39A, 39B. Hematological parameters before and after in vivo HSPC gene therapy of Hbbth3/CD46+/+ mice (week 16). (FIG. 39A) Reticulocyte counts. (FIG. 39B) Hematological parameters. Statistical analyses were performed using two-way ANOVA. *p<0.05; **p<0.0001

FIGS. 40A, 40B. Phenotypic correction of extramedullary hematopoiesis in spleen and liver. (FIG. 40Ai) Spleen size at sacrifice (week 16). Left panel: representative spleen images. Right panel: summary. Each symbol represents an individual animal. Statistical analysis was performed using one-way ANOVA. **p<0.0001. The difference between the two vectors is not significant. (FIG. 40B) Extramedullary hemopoiesis by hematoxylin/eosin staining in liver and spleen sections. Clusters of erythroblasts in the liver and megakaryocytes in the spleen of Hbbth3/CD46+/+ mice are indicated by black arrows. The scale bars are 20 μm. Representative images are shown.

FIG. 41. Phenotypic correction of hemosiderosis in spleen and liver (week 16). Iron deposition is shown by Perl's staining as cytoplasmic blue pigments of hemosiderin in spleen and liver sections. The scale bars are 20 μm. Representative sections are shown. (Exp: 2.24 ms, gain: 4.1×, saturation: 1.50, gamma: 0.60).

FIGS. 42A-42C. Analysis of bone marrow at sacrifice (week 21). Bone marrow was harvested at week 21 after in vivo HSC transduction of Hbb^{th3}/CD46tg mice. (FIG. 42A) Vector copy number per cell in bone marrow MNCs. (FIGS. 42B, 42C) Erythroid specificity of γ-globin expression. (FIG. 42B) Percentage of γ-globin expressing erythroid (Ter119⁺) and non-erythroid (Ter119⁻) cells. *p<0.05. Statistical analyses were performed using two-way ANOVA.

FIG. 43. Extramedullary hemopoiesis by hematoxylin/eosin staining in liver and spleen sections from CD46tg and CD46^{+/+}/Hbbth⁻³ mice prior to administration of an adenoviral donor vector. Iron deposition is shown by Perl's staining as cytoplasmic blue pigments of hemosiderin in spleen.

FIGS. 44A-44E. Phenotypic correction of CD46^{+/+}/Hbbth-3 mice by in vivo HSPC transduction/selection. (FIG. 44A) RBC analysis of healthy (CD46tg) mice, CD46^{+/+}/Hbbth-3 mice prior to mobilization and in vivo transduction, and CD46^{+/+}/Hbbth-3 mice that underwent in vivo transduction/selection (analyzed at week 29 after HDAd infusion) (n=5). *P≤0.05, **P≤0.0002, ***P≤0.00003. Statistical analysis was performed using 2-way ANOVA. (FIG. 44B) Supravital stain of peripheral blood smears with Brilliant cresyl blue for reticulocyte detection. Arrows indicate reticulocytes containing characteristic remnant RNA and micro-organelles. The percentages of positively stained reticulocytes in representative smears were: for CD46, 7%; for CD46^{+/+}/Hbbth-3 before treatment, 31%; and for CD46^{+/+}/Hbbth-3 after treatment, 12%. Scale bar: 20 μm. (FIG. 44C) Top: Blood smears. Scale bar: 20 μm. Middle: Bone marrow cytospins. Arrows indicate erythroblasts at different stages of maturation and a backshift in erythropoiesis with pro-erythroblast predominance in treated mice. Scale bar: 25 μm. Bottom: Tissue hemosiderosis by Perls' stain. Iron deposition is shown as cytoplasmic blue pigments of hemosiderin in spleen tissue sections. The blood smear images for the control mice (CD46tg and CD46^{+/+}/Hbbth-3, before transduction) in C and FIG. 5D are from the same sample. (FIG. 44D) Macroscopic spleen images of 1 representative CD46tg and 1 untreated CD46^{+/+}/Hbbth-3 mouse and 5 treated CD46^{+/+}/Hbbth-3 mice. (FIG. 44E) At sacrifice, spleen size was determined as the ratio of spleen weight to total body weight (mg/g). Each symbol represents an individual animal. Data are presented as means Å} SEM. *P≤0.05. Statistical analysis was performed using 1-way ANOVA.

FIG. 45. Cellular bone marrow composition of CD46 and treated Hbbth3/CD46 mice at week 16 after in vivo transduction. The differences between the groups were not significant. Statistical analyses were performed using two-way ANOVA.

FIG. 46. Human γ-globin gating strategy. Fixed and permeabilized RBCs from CD46/Hbbth3 mice were stained for the erythroid marker Ter-119 and intracellular γ-globin.

FIGS. 47A, 47B. Effect of SB100x-mediated integration on the transcriptome of CD34+ cells. (FIG. 47A) Schematic of experiment. CD34+ cells were infected with a HDAd5/35++ vector containing a GFP/mgmt cassette under control of the EF1α promoter alone or in combination with HDAd-SB. Transduced cells were expanded in erythroid differentiation medium for 16 days. Two rounds of O6BG/BCNU selection (50 μM O6BG+35 μM BCNU) enriched for GFP− positive cells with integrated transposons. At day 16, GFP-positive cells were FACS sorted (sample #6). For comparison (sample #5), CD34+ cells that were transduced with the mgmt/GFP vector alone and subjected to selection were used. Because the control cells did not express SB100x, they lost the episomal mgmt/GFP vector and were therefore GFP negative. Total RNA from both samples were subjected to RNA-Seq performed by Omega Bioservices. (FIG. 47B) Genes with altered mRNA expression (log 2 fold change) ranked based on their p value.

FIG. 48. mgmt mRNA expression levels in bone marrow MNCs at week 16 after in vivo transduction. Human mgmt^{P140K} and mouse mRPL10 levels were measured by qRT-PCR in total bone marrow MNCs. (mRPL10 is a mouse housekeeping gene). The relative levels were further divided by the VCN (see FIG. 33). Statistical analyses were performed using two-way ANOVA.

FIG. 49. In vivo HSC transduction in vector hCD46tg in mice: “long” vs “short” vectors LCR. In vivo transduction of vector Hbb^{th3}/CD46 in mice. Group 1 shows the in vivo transduction of HDAd-long-LCR-γ-globin/mgmt plus HDAd-SB/Flpe in seven mice. Group 2 shows the in vivo transduction of HDAd-short-LCR γ-globin/mgmt plus HDAd-SB/Flpe in three mice. Only three selection cycles were needed for O⁶BG, BCNU.

FIG. 50. Thbb mice test (W6). The graphical results show no difference and almost no human γ-globin expression among the mice when transduced with Long LCR vectors verses Short LCR vectors.

FIG. 51. Thbb mice test (W8). The graphical results show a difference among the mice when transduced with Long LCR vectors verses Short LCR vectors, however, it is unclear if Short LCR virus were dead in the mice.

FIG. 52. Graphic depiction showing the percentage of human γ-globin expressing RBC in mice. The graph illustrates 100% marking after only three cycles of in vivo selection.

FIG. 53. Graphic depiction of HPLC showing the relative human γ-globin to mouse HBA (week 10). The graph shows significantly higher γ-globin levels for long LCR compared to short LCR.

FIG. 54. Graphical depiction of example Week 10 blood HPLC of mouse #57 containing a Long LCR vector.

FIGS. 55A-55E. Characterization of the AAVS1-specific CRISPR/Cas9 vector and donor vector for HDR-mediated integration. (FIG. 55A) HDAd-CRISPR vector structure: The AAVS1-specific sgRNA is transcribed by Pall from the U6 promoter and the spCas9 gene is under the control of the EF1α promoter. Cas9 expression is controlled by miR-183-5p and miR-218-5p, which suppress Cas9 expression in HDAd producer 116 cells but do not negatively affect Cas9 expression in CD34+ cells (Sayadaminova et al., Mol Ther Methods Clin Dev, 1, 14057, 2015). The corresponding micro RNA target sites (miR-T) were embedded into a 3′ untranslated region of the β-globin gene (3′UTR). (FIG. 55B) Target site cleavage frequency in human CD34+ cells measured by T7E1 assay 3 days after HDAd-CRISPR transduction at a MOI of 2000 vp/cell. The specific cleavage products are 474 bp and 294 bp. The cleavage efficacy is shown below the gel. (FIG. 55C) Top 13 most frequent indels (SEQ ID NOs: 6-18, in order from top to bottom) found in HDAd-CRISPR-transduced CD34+ cells. The light grey highlighted sequence shows the target of the guide RNA with the TAM sequence marked in medium grey highlighting. The CRISPR/Cas9 cleavage site is marked by a vertical arrow. In green are insertion caused by NHEJ. (FIG. 55D) Structure of the donor vector for integration into the AAVS1 site (HDAd-GFP-donor). The mgmtP140K gene is linked to the GFP gene through a self-cleaving picornavirus 2A peptide. The genes are under the control of the EF1α promoter. PA: poly-adenylation signal. The transgene cassette is flanked by 0.8 kb regions of homology to the AAVS1 locus analogous to a previously published study (Lombardo et al., Nat Methods 8, 861-869, 2011). Upstream and downstream of the homology region are recognition sites for the AAVS1-specific CRISPR/Cas9 to release the donor cassette. (FIG. 55E) Release of the donor cassette. CD34+ cells were infected with the HDAd-GFP-donor (at MOIs of 1000 or 2000 vp/cell) alone or in combination with HDAd-CRISPR (MOI 1000 vp/cell). Three days later genomic DNA was subjected to Southern blot with a GFP-specific probe. The (linear) full-length HDAd-donor-GFP genome runs at 36 kb. The released cassette runs at 4.7 kb. The cleavage frequency is shown below the gel.

FIGS. 56A-56F. Targeted integration vs. SB100x-mediated integration in HUDEP-2 cells. (FIG. 56A) Experiment scheme. HUDEP-2 cells were transduced with the indicated HDAd vectors at a MOI of 1000 vp/cell for each virus. After expansion for 21 days, GFP positive cells were sorted into 96 well plate. Single cell-derived clones were obtained by further expansion for 2 weeks. GFP expression were measured at day 2 and 21 post transduction in the cell population, or at day 35 in cell clones. (FIG. 56B) GFP flow cytometry in cells treated with donor vector alone or vectors with targeted vs SB100x integration mechanisms at day 2 and 21. (FIG. 56C) Mean fluorescence intensity of GFP in total GFP⁺ cells with targeted vs SB100x integration (day 21). Data shown (mean±SD) represent three independent experiments. (FIG. 56D) Mean fluorescence intensity of GFP in single clones. Each symbol represents one cell clone. Data shown (mean±SD) are representative of two independent experiments. (FIG. 56E) Flow cytometry showing GFP expression in representative cell clones with targeted or SB100x-mediated integration. (FIG. 56F) Vector copy number in cell clones by qPCR using GFP primers.

FIGS. 57A, 57B. Integration analysis of HUDEP-2 clones transduced with targeted integration vectors. (FIG. 57A) Integration site analysis by inverse PCR. The upper diagram shows the locations of utilized NcoI sites, and primers (half arrows. dark gray: EF1α primers for 5′-junctions; light gray: pA primers for 3′ junctions). The expected amplicon size at each side for targeted integration is indicated. The lower gel pictures show iPCR results. Each lane represents one cell clone. The 1 kb ladder from New England Biolabs was used. An extra band of endogenous Ef1α was detected since Ef1α primers were adopted. For clone #20, although the amplicon size is different from prediction, cloning and sequencing revealed it is a clone with target integration. (FIG. 57B) In-Out PCR analysis. The upper diagram shows the location of primers. Expected product sizes for various integration patterns are listed. The lower gel pictures demonstrate that most clones had monoallelic targeted integration. With regard to the results from (FIG. 57A), the unexpected amplicon size from clones #17, #20 and #36 likely resulted from concatemeric integration.

FIGS. 58A-58C. Cleavage of AAVS1 target site in AAVS1/CD46tg mice. (FIG. 58A) In vitro analysis. Target site cleavage frequency in bone marrow lineage-negative cells from AAVS1/CD46tg mice measured 3 days after in vitro HDAd-CRISPR transduction at the indicated MOIs. (FIG. 58B) Percentage of total AAVS1 indels obtained by deep sequencing of DNA from total bone marrow mononuclear cells at week 14 after transplantation. Each symbol is an individual animal. (FIG. 58C) Top 29 most frequent indels (SEQ ID NOs: 19-23, 21, 21, 26-30, 27, 32, 28, 34-47), in order from top to bottom) found in a mouse. Representative data are shown. The yellow sequence shows the target of the guide RNA with the TAM sequence marked in blue. The CRISPR/Cas9 cleavage site is marked by a vertical arrow.

FIGS. 59A-59D. Ex vivo transduction of AAVS1/CD46 Lin− cells with HDAd-AAVS1 and HDAd-GFP-donor and subsequent transplantation into lethally irradiated recipients. (FIG. 59A) Schematic of the experiment: Bone marrow was harvested from AAVS1/CD46tg mice and lineage-negative cells (Lin−) were isolated by MACS. Lin− cells were transduced with HDAd-CRISPR and HDAd-GFP-donor alone or in combination at a total MOI of 500 vp/cell. After one day in culture, 1×10⁶ transduced cells/mouse were transplanted into lethally irradiated C57Bl/6 mice. At week 4, O⁶BG/BCNU treatment was started and repeated three times every two weeks. With each cycle, the BCNU concentration was increased from 5 mg/kg, to 7.5 mg/kg, to 10 mg/kg. At week 14, mice were sacrificed and bone marrow Lin− cells were used for transplantation into lethally irradiated secondary C57Bl/6 recipients, which were then followed for 16 weeks. (FIG. 59B) Percentage of GFP-positive cells in peripheral blood mononuclear cells (PBMCs) measured by flow cytometry. Shown are groups that were transplanted with Lin− cells transduced with HDAd-CRISPR only, HDAd-GFP-donor only, and HDAd-CRISPR+HDAd-GFP-donor. Each symbol represents an individual animal. (FIG. 59C) Percentage of GFP+ cells in PBMCs from representative mice transplanted with Lin− cells. Data from week 4 (before selection) and week 12 (after selection) are shown. (FIG. 59D) Percentage of GFP+ cells in lineage-positive cells CD3+ (T-cells), CD19+ (B-cells), Gr-1+ (myeloid cells), and in HSCs (LSK cells).

FIGS. 60A-60E. Analysis of engraftment of ex vivo transduced Lin− cells. (FIG. 60A) Engraftment of transplanted cells based on human CD46 expression on PBMCs measured by flow cytometry. Each symbol is an individual animal. Notably, transduced donor cells expressed CD46, while recipient C57Bl/6 mice did not. (FIG. 60B) Percentage of CD46-positive cells in PBMCs (blood), spleen, and bone marrow at week 14. (FIG. 60C) Percentage of GFP-positive cells in PBMCs, spleen and bone marrow, at week 14. (FIG. 60D) Percentage of LSK and lineage-positive cells in different transduction settings. The difference between the three groups is not significant. (FIG. 60E) Analysis of GFP+ colonies. Total bone marrow Lin− cells from week 14 mice were plated and GFP expression in colonies was analyzed 12 days later. Each symbol is the average GFP+ colony number for an individual mouse (left panels). Cells from all colonies were pooled and analyzed by flow cytometry (right panels).

FIGS. 61A-61F. Analysis of GFP marking in secondary recipients. Bone marrow cells from responder mice that were transplanted with HDAd-GFP-donor or HDAd-CRISPR+HDAd-GFP-donor transduced Lin− cells were harvested at week 14 after transplantation, depleted for lineage-positive cells, and transplanted into lethally irradiated C57Bl/6 mice. (FIG. 61A) GFP-flow cytometry of PBMCs in four recipient mice. The right panel shows a typical analysis. The vertical axis shows staining for hCD46, the horizontal axis shows GFP staining. (FIG. 61B) Percentage of GFP-positive cells in PBMCs, spleen and bone marrow, at week 16. (FIG. 61C) GFP flow analysis of lineage-positive and -negative cells in recipients 16 weeks after transplantation. (FIG. 61D) Analysis of GFP+ colonies. Total bone marrow Lin− cells from week 16 mice were plated and GFP expression in colonies was analyzed 12 days later. Each symbol is the average GFP+ colony number for an individual mouse (left panels). Cells from all colonies were pooled and analyzed by flow cytometry (right panels). (FIG. 61E) Engraftment of transplanted cells based on human CD46 expression on PBMCs measured by flow cytometry. (FIG. 61F) Percentage of lineage-positive and -negative cells in different transduction settings. The difference between the two groups is not significant.

FIGS. 62A-62F. In vivo transduction of AAVS1/CD46tg mice with HDAd-AAVSI-CRISPR+HDAd-GFP-donor. (FIG. 62A) Treatment regimen. AAVS1/hCD46tg mice were mobilized and IV injected with HDAd-CRISPR+HDAd-GFP-donor (2 times each 4×1010 vp of a 1:1 mixture of both viruses). Four weeks later, O6BG/BCNU treatment was started. With each cycle, the BCNU concentration was increased from 2.5 mg/kg, to 7.5 mg/kg, and 10 mg/kg. The O6BG concentration was 30 mg/kg in all three treatments. Mice were followed until week 12 when animals were sacrificed for analysis and Lin− cell transplantation into secondary recipients. Secondary recipients were then followed for 16 weeks. (FIG. 62B) Percentage of GFP-positive cells in peripheral blood mononuclear cells (PBMCs) measured by flow cytometry. (FIG. 62C) Percentage of GFP-positive cells in PBMCs, spleen and bone marrow, at week 14. (FIG. 62D) Percentage of GFP+ cells in lineage-positive cells CD3+ (T-cells), CD19+ (B-cells), Gr-1+ (myeloid cells), and in HSCs (LSK cells). (FIG. 62E) Analysis of GFP+ colonies. Total bone marrow Lin− cells from week 14 mice were plated and GFP expression in colonies was analyzed 12 days later. Each symbol is the average GFP+ colony number for an individual mouse (left panels). Cells from all colonies were pooled and analyzed by flow cytometry (right panels). (FIG. 62F) Percentage of lineage-positive and -negative cells at week 14.

FIGS. 63A-63E. Analysis of secondary recipients from FIG. 59A-59D. At week 14, bone marrow Lin− cells from in vivo transduced AAVS1/hCD46tg mice were transplanted into lethally irradiated C57Bl/6 recipients. (FIG. 63A) GFP-flow cytometry of PBMCs in six recipient mice. (FIG. 63B) GFP expression in mononuclear cells in blood, spleen and bone marrow. (FIG. 63C) GFP flow analysis of lineage-positive and -negative cells in recipients 16 weeks after transplantation. (FIG. 63D) Engraftment of transplanted cells based on human CD46 expression on PBMCs measured by flow cytometry. (FIG. 63F) Percentage of lineage-positive and -negative cells at week 16.

FIGS. 64A-64H. Ex vivo transduction of AAVS1/CD46 Lin− cells with HDAd-AAVS1 and HDAd-donor-γ-globin vectors and subsequent transplantation into lethally irradiated recipients. (FIG. 64A) Structure of the donor. The overall structure is the same as for the HDAds-GFP-donor vector (see FIG. 55D). The regions of homology are longer (1.8 kb vs 0.8 kb) in the new HDAd-globin-donor vector. The γ-globin expression cassette contains a 4.3 kb version of the γ-globin LCR including four DNAse hypersensitivity (HS) regions and the γ-globin promoter (Lisowski et al, Blood. 110, 4175-4178, 1996). The full length γ-globin cDNA including that 3′ UTR (for mRNA stabilization in erythrocytes) was used. The mgmtP140K gene is under the control of the ubiquitously active EF1α promoter. The bidirectional SV40 poly-adenylation signal is used to terminate transcription. To avoid interference between the LCR/β-promoter and EF1α promoter, a 1.2 kb chicken HS4 chromatin insulator (Emery et al., Proc Natl Acad Sci USA, 97, 9150-9155, 2000) was inserted between the cassettes. (FIG. 64B) The treatment regimen is the same as shown in FIG. 57A. (FIG. 64C) Percentage of human γ-globin-positive cells in peripheral red blood cells (RBCs) measured by flow cytometry. (FIG. 64D) Percentage and (FIG. 64E) mean fluorescence intensity of human γ-globin-positive cells in erythroid (Ter119+) and non-erythroid (Ter119−) cells in blood and bone marrow at week 16 after in vivo transduction. *p<0.05. (FIG. 64F) Percentage of γ-globin chains relative to mouse β-major chains measured in RBCs at week 16 by HPLC. (FIG. 64G) Percentage of γ-globin mRNA relative to mouse β-major RNA measured in RBCs at week 16 by qRT-PCR. (FIG. 64H) Vector copy number per cell in colonies derived from Lin− cells. Each symbol represents the one colony. Differences between animals are not significant.

FIGS. 65A, 65B. Engraftment of AAVS1/CD46 Lin− cells transduced with HDAd-CRISPR and HDAd-globin-donor vectors. (FIG. 65A) Engraftment of transplanted cells based on human CD46 expression on PBMCs measured by flow cytometry. (FIG. 65B) Percentage of CD46-positive cells in lineage-positive PBMCs (blood), spleen, and bone marrow cells as well as bone marrow LSK cells at week 16.

FIGS. 66A-66C. Analysis of secondary recipients from FIGS. 64A-64H. Bone marrow cells from mice that were transplanted with HDAd-CRISPR+HDAd-globin-donor transduced Lin− cells were harvested at week 16 after transplantation, depleted for lineage-positive cells, and transplanted into lethally irradiated C57Bl/6 mice. (FIG. 66A) γ-globin flow cytometry of RBCs in five recipient mice. (FIG. 66B) Percentage of CD46-positive cells in lineage-positive PBMCs. (FIG. 66C) Bone marrow composition at week 16 after transplantation into secondary recipients.

FIGS. 67A-67H. In vivo transduction of AAVS1/CD46tg mice with HDAd-CRISPR+HDAd-globin-donor. (FIG. 67A) Treatment regimen. (FIG. 67B) Percentage of γ-globin-positive RBCs. (FIG. 67C) Representative dot pot showing the percentage of γ-globin expression in peripheral RBCs from untransduced control mice or mice at week 16 after transduction. (FIG. 67D) Mean fluorescence intensity of γ-globin in erythroid (Ter119+) and non-erythroid (Ter119−) cells in blood and bone marrow. *p<0.05. (FIG. 67E) Percentage of γ-globin chains relative to mouse β-major chains measured in RBCs at week 16 by HPLC. *p<0.05. (FIG. 67F) Percentage of γ-globin mRNA relative to mouse β-major RNA measured in RBCs at week 16 by qRT-PCR. *p<0.05. (FIG. 67G) Vector copy number per cell in colonies derived from Lin− cells from four responder mice. Each symbol represents one colony. Differences between animals are not significant. (FIG. 67H) Composition of lineage-positive cells in blood, spleen and bone marrow and LSK cells in bone marrow at week 16 after in vivo transduction.

FIGS. 68A-68D. Analysis of secondary recipients from FIG. 67A-67H. (FIG. 68A) Engraftment of transplanted cells based on human CD46 expression on PBMCs measured by flow cytometry. (FIG. 68B) γ-globin expression in RBCs. (FIG. 68C) Percentage of γ-globin chains relative to mouse β-major chains measured in RBCs of secondary recipients at week 16 by HPLC. (FIG. 68D) Lineage-positive cell composition in blood, spleen and bone marrow at week 16 after in vivo transduction.

FIGS. 69A, 69B. Localization and structure of the AAVS1 locus in AAVS1/CD46 transgenic mice. (FIG. 69A) TLA data showing mismatches on chromosome 14. An AAVS1-specific primer pair was used. The right panel shows an enlarged section of chromosome 14 with the 18 kb gap visible. The gap corresponds to the added human AAVS1 loci. (FIG. 69B)

FIG. 70. Detailed structure of the AAVS1 loci indicating the genomic localization. The shaded AAVS1 areas were confirmed by Sanger sequencing. The empty areas were deducted from restriction analysis and AAVS1 tg mice genetic background information from The Jackson Laboratory. The CRISPR/Cas9 cleavage sites are indicated by scissors. Repeats #2 to #5 are complete 8.2 kb human AAVS1 EcoRl fragments, while repeats #1 and #5 only contain only a fraction of the EcoRl fragment. Notably, repeat #5 lacks a complete 5′ homology arm. Outcome depending on CRISPR/Cas9 cleavage of the multicopy AAVS1 locus present in AAVS1tg mice. Rules regarding cutting positions are as follows: a) One single cut in repeat #1 to #4: preferred. b) One single cut in repeat #5: reduced preference due to incomplete left homology arm. c) Two cuts in two oppositely oriented repeats (e.g. #1 and #4): no HDR-mediated targeted integration due to missing right homology arm. d) Two cuts in two repeats facing the same direction (e.g. #1 and #2): preferred. e) For more than 2 cuts, only consider the one proximal to mouse gDNA sequence at each side: Apply rule c) or d) accordingly. f) Cuts in repeats #1 and #5 and deletion of the central region. In addition, HDR-mediated targeted integration occurred in repeat #2 to #4, continuous cutting in flanking repeats, for example #1 and #5, by CRISPR may result in loss of the already integrated transgene.

FIGS. 71A, 71B. Integration site analysis by Southern of genomic DNA isolated at week 16 after ex vivo or in vivo HSC transduction with HDAd-CRISPR+HDAd-GFP-donor. (FIG. 71A) Hybridization with an AAVS1-specific probe. The upper panel shows the expected EcoRl fragment size and the localization of the probe. The lower panel shows the analysis of individual mice from ex vivo and in vivo transduction setting. The larger bands represent non-targeted AAVS1 loci repeats. (FIG. 71B) Hybridization of BlpI-digested DNA with a GFP-specific probe. The band pattern is discussed elsewhere.

FIGS. 72A-72C. Integration site analysis by inverse PCR (iPCR) of genomic DNA isolated at week 16 after ex vivo or in vivo HSC transduction with HDAd-CRISPR+HDAd-GFP-donor. (FIG. 72A) The diagram shows the locations of NcoI sites, and primers (half arrows: EF1α primers for 5′ junctions; light gray: pA primers for 3′ junctions). The expected amplicon size at each side for targeted integration in repeat #5 is indicated. (FIG. 72B) iPCR results using genomic DNA from total bone marrow cells. Each lane represents one mouse. #009, #023, #943, #944 and #946 are mice after ex vivo HSC transduction. #147, #304 and #467 are in vivo transduced animals. (FIG. 72C) iPCR analysis of GFP-positive colonies. Bone marrow Lin− cells from week 14 mice were plated, genomic DNA was isolated from GFP+ colonies 20 days later and used for iPCR. Mice #943 and #946 were analyzed. Each lane represents one colony. Light gray arrow: targeted integration; dark gray arrow: off-target integration; medium gray arrow: integrated whole HDAd viral genome.

FIGS. 73A, 73B. Integration site analysis by inverse PCR (iPCR) of genomic DNA isolated at week 16 after ex vivo or in vivo HSC transduction with HDAd-CRISPR+HDAd-globin-donor. (FIG. 73A) The diagram shows the locations of NcoI sites, and primers (half arrows. black EF1α primers for 5′ junctions; gray: pA primers for 3′ junctions). The expected amplicon size at each side for targeted integration in repeat #5 is shown. (FIG. 73B) iPCR results using genomic DNA from total bone marrow cells. Each lane represents one mouse. #321, #322, #856, #857, #858 and #945 are mice with ex vivo transduction. #504, #816 #869 and #898 are in vivo transduced animals. White arrowhead indicates targeted integration; Gray, dotted lined arrowhead: off-target integration; white full arrow: integrated whole HDAd viral genome.

FIGS. 74A-74D. (FIG. 74A) HDAd5/35++ vectors for in vivo HSPC transduction. In HDAd-GFP/mgmt, the transposon is flanked by inverted transposon repeats (IR) and frt sites for integration through a hyperactive Sleeping Beauty transposase (SB100X) provided from the HDAd-SB vector. The transgene cassette contains a PGK-promoter driven GFP gene linked to a β-globin 3′UTR as well as an EF1α-promoter driven mgmtP140K cassette. Both cassettes are separated by a chicken globin HS4 insulator. HSPCs were mobilized in neu/CD46 transgenic mice by s.c. injections of human recombinant G-CSF (5 μg/mouse/day, 4 days) followed by an s.c. injection of AMD3100 (5 mg/kg) eighteen hours after the last G-CSF injection. A total of 8×1010 viral particles of HDAd-GFP/mgmt+HDAd-SB were injected i.v. one hour after AMD3100. To prevent pro-inflammatory cytokine release after HDAd injection, animals received Dexamethasone (10 mg/kg) i.p. 16 h and 2 h before virus injection. Six weeks later, three rounds of O6BG/BCNU (i.p.) were applied to activate the exit of transduced HSPCs into the peripheral blood circulation (30 mg/kg O6BG plus 5, 7.5, and 10 mg/kg BCNU). Seventeen weeks after in vivo transduction, 1×10⁶ MMC cells were implanted into the mammary fat pad. Five weeks later, tumors and other tissues were harvested and analyzed for GFP expression. (FIG. 74B) Left Panel: Percentage of GFP-expressing PBMCs at different time points after in vivo transduction. Each symbol represents an individual animal. Right Panel: Percentage of GFP+ cells in cells stained for the pan-leukocyte marker CD45 in bone marrow, spleen, blood, and collagenase/dispase-digested tumor. (FIG. 74C) Tumor section stained with an antibody against GFP and an antibody against laminin, an extracellular matrix protein. The scale bar is 50 μm. (FIG. 74D) Immunophenotyping of GFP+PBMCs in the blood and of GFP+ cells in the tumor.

FIG. 75. Rat Neu expression in MMC cells. Cells were stained with the Neu-specific monoclonal antibody 7.16.4 followed by anti-mouse Ig-FITC. Shown is a representative confocal microscopy image of cultured MMC cells. New-Specific signals appear in whiter hues. The scale bar is 20 μm.

FIG. 76. Gating strategy for immunophenotyping.

FIG. 77. Immunophenotyping of GFP+ cells in the bone marrow and spleen (MMC model). For details, see FIG. 74D.

FIGS. 78A-78F. GFP expression in tumor-infiltrating leukocytes after in vivo HSPC transduction (TC-1 model). (FIG. 78A) Schematic of the experiment. HSPCs were mobilized in CD46tg transgenic mice by s.c. injections of human recombinant G-CSF (5 mg/mouse/day, 4 days) followed by an s.c. injection of AMD3100 (5 mg/kg) eighteen hours after the last G-CSF injection. A total of 8×1010 viral particles of HDAd-GFP/mgmt+HDAd-SB were injected i.v. one hour after AMD3100. To prevent pro-inflammatory cytokine release after HDAd injection, animals received Dexamethasone (10 mg/kg) i.p. 16 h and 2 h before virus injection. Six weeks later, three rounds of O⁶BG/BCNU (i.p.) were applied to activate the exit of transduced HSPCs into the peripheral blood circulation (30 mg/kg O6BG plus 5, 7.5, and 10 mg/kg BCNU. 17 weeks after in vivo transduction, 5×10⁴ TC-1 cells were implanted into the mammary fat pad. Five weeks later, tumors and other tissues were harvested and analyzed for GFP expression. (FIG. 78B) Percentage of GFP-expressing PBMCs at different time points after in vivo transduction. Each symbol represents an individual animal. (FIG. 78C) Percentage of GFP+ cells in cells stained for the panleukocyte marker CD45 in bone marrow, spleen, blood, and collagenase/dispase-digested tumor. (FIG. 78D) Representative flow cytometry data of GFP+ cells in total (malignant+tumor infiltrating) cells and of GFP+positive leukocytes. (FIG. 78E). Representative tumor section. Left panel: GFP fluorescence. Right panel: staining with antibodies against GFP (white) and the extracellular matrix protein laminin (gray). The scale bar is 50 mm. (FIG. 78F) Immunophenotyping of GFP+ cells in the tumor and PBMCs in blood. Lymphocyte flow cytometry panel 8c (CD45, CD3, CD4, CD8, CD25, CD19) and myeloid panel 9c (CD45, CD11c, F4/80, MHCII, SiglecF-PecCP, Ly6C, CD11b, Ly6G) from BD Biosciences were used.

FIGS. 79A-79C. Selection of miRNAs for suppression in cells other than tumor-infiltrating leukocytes. (FIG. 79A) miRNA-based regulation of tissue-specificity of transgene expression. miRNAs function as guide molecules through base pairing with target sequences, referred to as miRNA Target Sites (miR-T), typically residing in the 3′ untranslated region (3′ UTR) of native mRNAs. This interaction recruits effector complexes mediating mRNA cleavage or translational repression. If the mRNA of a transgene contains miR-Ts for a miRNA that is expressed at high levels in a given cell type, transgene expression will be prevented in this cell type. In contrast, in cell types that do not express the specific miRNA, the transgene will be expressed (Brown et al., Nat Med. 2006; 12: 585-591). (FIG. 79B) MicroRNA-Seq was performed on RNA pooled from five mice (neu/CD46tg-MMC model, day 17 after tumor inoculation). Shown are normalized microRNA read counts (reads per million mapped microRNAs+1) identified by small RNA sequencing of spleen, bone marrow and blood versus GFP⁺ tumor 13 samples. MicroRNAs that are not present in the tumor, including miR-423, align at the left of the scatterplot with a pseudo-count of 1. miR-423-5p is indicated in the blot. (FIG. 79C) MicroRNA-Seq was performed on RNA pooled from five mice (CD46tg/TC-1 model, day 17). Relative expression level of the top 10 miRNAs compared to levels in the tumor (set to 1).

FIGS. 80A-80C. Effect of miR-423-5p target site overexpression on HSPCs. (FIG. 80A) Vector structure. HDAd-GFP-miR-423 contains four miR-423-5p target sites in the 3′UTR linked to the GFP gene. (FIG. 80B) Mouse HSPCs (M) (Lin− cells from the bone marrow of CD46-transgenic mice) and human HSPCs (Hu) (CD34+ cells) were infected with either HDAd-GFP or HDAd-GFP-miR423 at a MOI of 500 or 3000 vp/cell, respectively. Three days later, cell lysates were analyzed by Western blot for CDKNIA. Blots were re-probed with anti-β-actin antibodies to adjust for loading differences. The right panel shows the quantification of CDKNIA signals normalized to b-actin signals. The signals from the corresponding mouse and human HDAd-GFP/mgmt samples were taken as 100%. (FIG. 80C) Effect on progenitor colony formation. One day after HDAd infection, mouse Lin⁻ cells (2.5×10³ cells per 35 mm dish) or human CD34+ cells (3×10³ cells/dish) were plated for colony assays. Colonies were counted 12 days later. N=3. *p<0.05. Statistical significance was calculated by two-sided Student's t-test (Microsoft Excel). (In agreement with previous studies (Li et al., Mol Ther Methods Clin Dev. 2018; 9: 390-401; Li et al., Mol Ther Methods Clin Dev. 9: 142-152, 2018), infection of HSPCs at relatively high MOIs slightly reduced the colony forming capacity of HSPCs.)

FIG. 81. Validation of miR-423-5p expression by Northern blot. Total RNA (2 μg) from bone marrow lineage-negative cells, spleen, total blood cells, and MMC-/TC-1-tumor infiltrating leukocytes was separated in 15% denaturing polyacrylamide gel and blots were hybridized with a probe specific for muRNA-423-5p and subsequently with a probe for U6 RNA (as loading control). Mir-423 has a precursor length of 70 bp and a mature miRNA length of 23 bp. miR-423-5p-specific signals are visible for blood, bone marrow, and spleen, but absent in tumor-infiltrating cells in both tumor models.

FIGS. 82A, 82B. miRNA423-5p expression in humans. (FIG. 82A) Levels of miR-423-5p published in Ludwig et al., Nucleic Acids Res. 2016; 44: 3865-3877. From left to right, y-axis label includes: adipocyte, artery, colon, dura mater, kidney, liver, lung, muscle, myocardium, skin, spleen, stomach, testis, thyroid, small intestine duodenum, small intestine jejunum, pancreas, kidney glandula suprarenalis, kidney cortex renalis, kidney medulla renalis, esophagus, prostate, bone marrow, vein, lymph node, pleura, brain pituitary gland, spinal cord, brain thalamus, brain white matter, brain nucleus caudalus, brain gray matter, brain cerebral cortex temporal, brain cerebral cortex frontal, brain cerebral cortex occipital, and brain cerebellum. (FIG. 82B) Plotted miRNA-Seq data from two ovarian cancer patients (pooled). CD45+ cells were isolated from biopsies of high-grade serous ovarian. RNA was isolated from tumor-infiltrating leukocytes and matching PBMCs and subjected to miRNA-Seq by LC Sciences, LLC. miRNA-423-5p is indicated.

FIGS. 83A-83E. In vivo HSPC αPD-L1-γ1 immune-checkpoint inhibitor therapy in the neu/MMC model. (FIG. 83A) PDL1 expression (white) in MMC tumor cells. The scale bar is 20 μm. (FIG. 83B) The overall structure of the therapy vector is the same as shown in FIG. 74A. The vector contains the expression cassettes for a scFv anti-mouse PD-L1 linked to a HA tag and secretion signal (LS) on the 5′ end and to the hinge-CH2-CH3 domains of human IgG1 and myc tag on the 3′ end. miR423-5p target sites were inserted into the 3′UTR to restrict αPD-L1-γ1 expression to tumor-infiltrating cells by miR423-5p regulation. The vector also contains an expression cassette for mgtm^{P140K}. (FIG. 83C) Tumor volumes after MMC cell inoculation (day 0) in mice with HDAd-GFP/mgmt and HDAd-αPD-L1-γ1 in vivo transduced HSPCs. Mice in the HDAd-αPD-L1-γ1 group were re-challenged by a subcutaneous injection of 1×10⁵ MMC cells at day 80 after the first tumor cell injection. Each curve is an individual animal. (FIG. 83D) Analysis of T-cell responses by flow cytometry. Splenocytes from naïve neu-transgenic mice and HDAd-αPD-L1-yl-treated mice (day 100) were analyzed by flow cytometry for CD4, CD8, and intracellular IFNγ or stained with the Neu tetramer. N=3. *p<0.05. (FIG. 83E) IFNγ response upon stimulation with Neu+ and Neucells. Splenocytes from naïve neu-transgenic mice and HDAd-αPDL1-γ1-treated mice (day 100) were exposed to arrested MMC cells (Neu+) or splenocytes from neutransgenic mice (Neu-), or treated with PMA/ionomycin (“noAg”). Shown is the IFNγ concentration in culture supernatants. N=3. *p<0.005.

FIGS. 84A-84C. Kinetics of αPD-L1-γ1 expression. (FIG. 84A) αPD-L1-γ1 Western blot with anti-HA tag antibodies. Three animals were sacrificed at day 17 and tissues were analyzed for αPD-LI-γ1 expression by Western blot. αPD-L1-γ1 protein was not completely reduced, resulting in remnants of complete αPD-L1-γ1 with two scFv chains (130 kDa) (see right panel for the structure of αPD-L1-yl). Staining for β-actin was used for loading controls. Shown are representative samples. Also shown is quantification of Western blot signals. N=5 mice. (FIG. 84B) αPD-L1-γ1 mRNA expression in tumor-infiltrating leukocytes, PBMCs, bone marrow cells and splenocytes. Mouse PPIA mRNA was used as an internal control. Results were calculated according to the 2(−ΔΔCt) method and presented as percentage of relative expression, with setting the cDNA level of corresponding tumor samples as 100%. (FIG. 84C) Levels of secreted αPD-L1-γ1 in the serum measured by ELISA using recombinant mouse PD-L1 for capture and an anti-HA antibody-HRP conjugate for detection. Each symbol represents an individual animal. *p<0.05. Statistical significance was calculated by two-sided Student's t-test (Microsoft Excel).

FIGS. 85A-85F. Immuno-prophylaxis study in the ID8-p53^{-/-} brca2^{-/-} ovarian cancer model. (FIG. 85A) Analysis of ID8-p53^{-/-} brca2^{-/-} tumors. A total 2×10⁶ ID8-p53^{-/-} brca2^{-/-} cells were injected intraperitoneally into CD46-transgenic mice. Ascites/cachexia developed 6-8 weeks later. Tumors were then removed and digested with dispase/collagenase for flow cytometry. A fraction of cells was sorted for tumor-associated macrophages (TAMs), neutrophils (TANs), and T-cells (TILs) for Northern blot analysis. (see FIG. 76). (FIG. 85B) Immunophenotyping of tumor-associated leukocytes. (FIG. 85C) Northern blot for miR-423-5p. A total of 1 μg of RNA was loaded per lane. The upper panel shows signals after probing with a ³²P-labeled miR-423-5p probe. The blot was stripped and re-probed with a U6 RNA specific probe (lower panel). The ³²P-labeled Decade marker from Ambion was run in the right lane. (FIG. 85D) Experimental scheme. CD46-transgenic mice were mobilized and injected either with HDAd-αPDL1γ1miR423+HDAd-SB, HDAd-GFP-miR423+HDAd-SB, or mock-injected. Four rounds of O⁶BG/BCNU in vivo selection were given. ID8-p53^{-/-} brca2^{-/-} cells were injected intraperitoneally two weeks after the last O⁶BG/BCNU treatment. Two, six, and eleven weeks after tumor cell injection, αPDL1γ1 levels were analyzed in serum. The onset of ascites or morbidity/cachexia were taken as endpoints. (FIG. 85E) Kaplan-Meier survival plot. N=7. (FIG. 85F) Serum αPDL1γ1 levels measured by ELISA. Each symbol is an individual animal. *p<0.05. Statistical significance was calculated by two-sided Student's t-test (Microsoft Excel).

FIGS. 86A-86D. Immuno-therapy study in the ID8-p53^{-/-} brca2^{-/-} ovarian cancer model. (FIG. 86A) Clinical setting to prevent cancer recurrence. In vivo HSC transduction will start after surgical tumor debulking or, if surgery is not an option, together with chemotherapy. O⁶BG/BCNU in vivo selection can be combined with chemotherapy. As a result of in vivo HSPC transduction/selection, armed HSPCs will lay dormant until cancer recurs which will trigger HSPC differentiation and activation of effector gene expression. (FIG. 86B) Experimental scheme. CD46 transgenic mice were intraperitoneally injected with 1×10⁶ ID8-p53^{-/-} brca2^{-/-} tumor cells. Once tumors were established, in vivo HSPC transduction and selection were performed. Activation of miR-423-based expression system was monitored based on serum αPDL1γ1 levels. (FIG. 86C) Kaplan-Meier survival plot. In the control setting, HDAd-GFP-miR423 was injected. N=9. (FIG. 86D) Serum αPDL1γ1 levels were measured by ELISA. Each symbol is an individual animal. *p<0.05. Statistical significance was calculated by two-sided Student's t-test (Microsoft Excel).

FIGS. 87A, 87B. Autoimmune reactions in animals sacrificed at day 17 at the peak of αPD-L1-γ1, before reversal of tumor growth. (FIG. 87A) Fur discoloration in a treated animal (right panel) compared to an animal before treatment (left panel). (FIG. 87B) Histological analysis of organs from a treated animal. Sections were stained with H&E. Shown are representative areas. The scale bar is 20 mm. Note the infiltrates of mononuclear cells.

FIGS. 88A-88H. Effect of anti-PD-L1 monoclonal antibody therapy in neu-transgenic mice with MMC tumors and effect of in vivo HSC transduction on hemopoiesis. When tumors reached a volume of 100 mm³, mice received intraperitoneal injections of the anti-mouse PD1-L1 monoclonal antibody muDX400* (5 mg/kg i.p.) (4× every 4 days) or an isotype control antibody. (FIG. 88A) Shown is the tumor volume in individual mice. (FIG. 88B) Kaplan-Meier survival plot, showing longer survival with anti-PD-L1. Tumors with a volume of 1000 mm³ were taken as endpoint. The difference between the two groups is not significant. (FIG. 88C) Blood cell counts in hCD46-transgenic mice shown in FIG. 85D at week 2 after in vivo HSCPC transduction (FIG. 85A) Hematological parameters. RBC: red blood cells, Hb: hemoglobin, MCV: mean corpuscular volume, MCH: mean corpuscular hemoglobin, MCHC: mean corpuscular hemoglobin concentration, RDW: red cell distribution width. Statistical analysis was performed using two-way ANOVA. The differences between the three groups were not significant. (FIG. 88E) niRNA-Seq of GFP+ cell fractions. (FIG. 88F) Kinetics of αPDL1 expression by western blot, qRT-PCR, and serum ELISA. (FIG. 88G) miRNA-regulated gene expression. (FIG. 88H) a summarized schematic of disclosed immune-prophylactic and cancer recurrence prevention.

FIGS. 89A-89H. Data related to GFP expression from erythrocytes.

FIGS. 90A-90I. Data related to human factor VIII expression from erythrocytes.

FIGS. 91A-91D. No hematological abnormalities are observed.

FIGS. 92A-92G. Phenotypic correction of hemophilia A in spite of inhibitor antibodies.

FIGS. 93A-93E. In vivo transduction in macaques (M. fascicularis). (FIG. 93A) experimental timeline; (FIGS. 93B-93D) GFP marking in mobilized CD34+ cells in peripheral blood; (FIG. 93E) bone marrow (Day 3).

FIGS. 94A-94M. Combined in vivo HSC. transduction selection. mgmt^{P140K} provides a mechanism for drug resistance and the selective expansion of gene-modified cells. (P140K mutant of human O(6)-methylguanine-DNA-methyltransferase (MGMT) confers resistance to the MGMT inhibitor O(6)-(4-bromothenyl) guanine (O6BG) also known as benzylguanine. (FIG. 94A) Vector for MGMT^{p140k}. (FIG. 94B) Experimental design showing timeline and dosages for injections. (FIG. 94C) Data showing percent of GFP+ cells in PBMC. (FIG. 94D) Data showing percent of GFP+ cell in bone marrow at week 26. (FIG. 94E) Ad5/35-GFP vector. (FIG. 94F) Experimental protocol depicting Pigtail macaques received 4 days of mobilization followed by Ad5/35 injection. (FIG. 94G) Animal IDs and doses of G-CSF, SCF, AMD3100, and Ad5/35-GFP. (FIG. 94H) AMD3100 increased total CD34+ stem cell levels three-fold better than G-CSF/SCF alone and 65-fold over baseline; left panel showed percentage of CD34+ stem cells in peripheral blood; right panel shows CD34+ cell counts. (FIG. 94I) Mobilized cells after AD5/35 injection form healthy colonies without lineage skewing; left panel provides numerical data showing the frequency and number of colonies zero to six hours post Ad5/35 injection; right panel provides visual inspection of morphology of CD34+ cells. (FIG. 94J) Top panel shows flow cytometry data of the Ad5/35-GFP cells from zero to 6 hours post injection. Bottom panel shows the numerical data of the number of colonies containing Ad5/35-GFP at zero, two, and six hours post injection. (FIG. 94K) Over 3% of peripheral CD34+ cells express GFP following Ad5/35 injection. Top panel depicts C34+ cells extracted from the mononuclear cell (MNC) layer from zero to 8 days post Ad5/35 injection. Bottom panel depicts the average GFP⁺ expression 2 and 6 hours post injection. (FIG. 94L) Multiple methods confirm successful transduction of circulating cells after mobilization and Ad5/35 injection. Left panel depicts Taqman detection of vector DNA. Right panel depicts flow cytometry data of GFP expression. (FIG. 94M) Modified cells home back to bone marrow. Left panel depicts flow cytometry data showing the change in CD34+ and GFP+ cells at day three, seven, and 73 post Ad5/35 injection. Right panel depicts the percent of GFP+, CD34+ cells at baseline, and three, seven, and 73 days post Ad5/35 injection.

FIG. 95. Features of representative Ad35 helper virus and vectors described herein. The five-point star indicates the following text: —combination (addition and reactivation) for SB100x and targeted; —multiple sgRNAs for CRISPR or BE; —miRNA (miR187/218) regulated expression of Cas9; and -auto-inactivation of Cas9.

FIG. 96. Schematic of HDAd-Tl-combo vector. The CRISPR system targets two different sites (HBG promoter and erythroid bcl11a enhancer), which leads to increased gamma reactivation.

FIGS. 97A-97D. (FIG. 97A). Upon co-infection of HDAd-SB and HDAd-combo, Flpe will be expressed and release the IR-flanked transposon, which will then be integrated into the genome by SB100x transposase. Simultaneously, HBG1 and bcl11a-E CRISPRs will be expressed and generate DNA indels that will lead to reactivation of [symbol] -globin. Upon Flp-mediated release of the transposon, the CRISPR cassette will be degraded, thereby avoiding cytotoxicity. The CRISPR system targets two different sites (HBG promoter and erythroid bcl11a enhancer), which leads to increased γ reactivation. (FIG. 97B) targeting strategy; (FIG. 97C) erythroid specific BCL11A enhancer; (FIG. 97D) BCL11A binding site at HBG promoter (SEQ ID NO: 48). Schematic of HDAd-SB and HdAd-comb-SB can be found in FIG. 102.

FIGS. 98A-98N. Dual CRISPR vectors and γ-globin reactivation. (FIG. 98A) Vector designs for HDAd-Bclllae-CRISPR, HDad-HBG-CRISPR, HDAd-Dual-CRISPR, and HDAd-scrambled. (FIG. 98B) HD-Ad5/35++CRISPR Vectors for dual gRNA vector. (FIG. 98C) HD-Ad5/35++CRISPR transduction of a human erythroid progenitor cell line (HUDEP-2) is shown before and after differentiation. The timeline is shown below HUDEP-2 cell images. (FIG. 98D) The HD-AD5/35++“Dual” gRNA vector does not negatively affect cell viability compared to untreated (UNTR), BCL11A, or HBG vectors. (FIG. 98E) The HD-AD5/35++“Dual” gRNA vector does not negatively affect proliferation compared to UNTR, BCL11A, or HBG vectors. (FIG. 98F, FIG. 98G) The Dual vectors achieve similar editing levels similar to those observed with the single gRNA vectors for the target loci (FIG. 98F) Bcl11a enhancer and (FIG. 98G) HBG promoter. (FIG. 98H) The HD-AD5/35++“Dual” gRNA vector achieves editing levels of target loci similar to those observed with the single gRNA vectors. (FIG. 98I) A significantly higher percentage of HbF+ cells were observed by flow cytometry in HUDEP-2 cells transduced with the HD-Ad5/35 “Dual” gRNA vector compared to the single gRNA vectors. A bar chart summarizing flow cytometry data is below the flow cytometry data. (FIG. 98J) The overall gamma globin expression, measured by HPLC, was significantly higher in the dual targeted samples. (FIG. 98K) A significantly higher fetal globin expression in double knock-out clones than single knock-out clones was observed implying a possible synergistic effect of the two mutations, leading to higher gamma expression/cell. (FIG. 98L) Schematic shows that peripheral blood mobilized CD34+ cells were transduced with the HDAd5/35++ CRISPR vectors. To minimize CRISPR/Cas9 cytotoxicity, cells were subsequently transduced with an HDAd5/35++ vector that expresses anti-Cas9 peptides. Cells were transplanted into sub-lethally irradiated NSG mice and analyzed. (FIG. 98M) At week 10 after transplantation, cells transduced with the HD-Ad5/35 “Dual” gRNA vector exhibited similar engraftment to the cells transduced with the single gRNA vectors. Lineage composition was similar in all groups. (FIG. 98N) CD34+ cells transduced and edited by the double gRNA vector, efficiently engrafted in NSG mice. Furthermore, the engrafted dual targeted cells after erythroid differentiation expressed higher levels of gamma globin to the control, compared to the single targeted cells, despite the relatively lower editing levels.

FIGS. 99A-99U. Ex vivo transduction of double edited normal and that CD34+ cells. (FIG. 99A) Experimental design. (FIG. 99B) HBF expression and (FIG. 99C) MFI in colonies on day 15 for normal CD34+ cells. * indicates that p=0.034. (FIG. 99D) Flow cytometry data describing HBF expression in colonies on day 15 in normal CD34+ cells. (FIG. 99E) HBF expression and (FIG. 99F) MFI after erythroid differentiation (ED) for normal CD34+ cells. * indicates that p=0.01. (FIG. 99G) TE71 for HBG site and (FIG. 99H) TE71 for BCL11A site 48 hours post transduction (txd) in normal CD34+ cells. (FIG. 99I) Flow cytometry data describing HBF expression in EC and erythroid differentiation. (FIGS. 99J-99U) ThaI CD34+ cells. (FIG. 99J) Immunophenotype of cells at day 0, untransduced cells and cells transduced with CRISPR-Dual and (FIG. 99K) a growth curve comparing untransduced cells and cells transduced with CRISPR-Dual over 11 days. (FIG. 99L) HBF expression and (FIG. 99M) MFI in colonies on day 15. ** indicates that p=0.0046. (FIG. 99N) HBF expression in erythroid and myeloid compartment comparing CRISPR-Dual versus untransduced cells. (FIG. 99O) HBF expression in erythroid and myeloid compartment comparing CRISPR-Dual A and B versus untransduced cells. (FIG. 99P) HBF expression in EC and (FIG. 99Q) MFI. *** indicates that p=0.0003 and **** indicates that p=0.00003. (FIG. 99R) Flow cytometry data describing HBF expression at PO4 and P18. (FIGS. 99S, 99T) TE71 for HBG site erythroid differentiation at (FIG. 99S) p04 and (FIG. 99T) p18. (FIG. 99U) TE71 for BCL11A site 48 hours after transduction.

FIG. 100. Graphical summary describing the combination of γ-globin gene addition and re-activation of endogenous γ-globin.

FIG. 101. HDAd5/35++ vectors used herein. γ-globin gene addition is achieved through the SB100x transposase system consisting of a transposon vector with IRs and frt sites flanking the expression cassette (see HDAd-combo and HDAd-SB-addition) and a second vector (HDAd-SB) that provides the SB100x and Flpe recombinase in trans. The transposon cassette for random integration consists of a mini β-globin LCR/promoter for erythroid specific expression of human γ-globin. The 3′UTR serves for mRNA stabilization in erythroid cells. The γ-globin expression unit is separated by a chicken globin HS4 insulator from a cassette for mgmt^{P140K} expression from a ubiquitously active PGK promoter. The CRISPR/Cas9 cassette in the HDAd-CRISPR and HDAd-combo vectors contains a U6 promote-driven sgRNA specific to the BCL11A binding site within the HBG1/2 promoter, a SpCas9 under EF1 a promoter control. Expression of Cas9 in HDAd producer cells is suppressed by a miRNA regulation system (Saydaminova et al., Mol Ther Methods Clin Dev. 2015, 1: 14057, 2015). In HDAd-combo, the CRISPR/Cas9 cassette is placed outside the transposon so that it will be lost upon Flpe/SB100x-mediated integration (see FIG. 102).

FIG. 102. Schematic for controlled Cas9 expression. In HDAd-combo, the interaction of Flpe recombinase with the frt sites leads to a circularization of the transposon, leaving linear fragment of the vector containing the CRISPR cassette. Previous studies with the SB100x/Flpe system demonstrated that these vector parts are rapidly lost while the circularized transposon is integrated into the host genome by SB100x (Yant et al., Nat Biotechnol., 20: 999-1005, 2002).

FIGS. 103A-103D. In vitro studies with HUDEP-2 cells to analyze Cas9 and γ-globin expression. (FIGS. 103A and 103B) Analysis of Cas9 expression by Western blot. HUDEP-2 cells were transduced with HDAd-combo alone and in combination with HDAd-SB (i.e. the vector that provides Flpe and SB100x in trans). In vitro erythroid differentiation was started 4 days post transduction and continued for 8 days. (Erythroid differentiation allows for γ-globin expression). Right panel: representative Western blot using Cas9 and β-actin antibodies as probes. Left panel: Summary of the Cas9 signals. The bars compare Cas9 with and without HDAd-SB coinfection, i.e. the reduction of Cas9 by the Flpe/SB100x mechanism. (FIG. 103C) Analysis of γ-globin expression by flow cytometry. HUDEP-2 cells were transduced with HDAd-CRISPR (“cut”), HDAd-SB-add (“add”)+HDAd-SB, or HDAd-combo (“combo”)+HDAd-SB and analyzed at the indicated time points. (FIG. 103D) γ-globin mRNA levels by qRT-PCR. d.p.t., days post transduction. Diff, differentiation. *p<0.05

FIGS. 104A-104H. γ-globin expression studies after in vivo transduction of CD46/f3-YAC mice. (FIG. 104A) Schematic of the experiment. HSPCs were mobilized by subcutaneous (s.c.) injections of human recombinant G-CSF for 4 days followed by one s.c. injection of AMD3100. 30 and 60 minutes after AMD3100 injection, animals were intravenously injected with a 1:1 mixture of the following HDAd vectors (2 injections, each 4×10¹⁰ vp): HDAd-combo+HDAd-SB, HDAd-SB-add+HDAd-SB, and HDAd-cut. Mice were treated with immunosuppressive (IS) drugs for the next 4 weeks to avoid immune responses against the human γ-globin and MGMT. At week 4, 0⁶-BG/BCNU treatment was started and repeated every 2 weeks for 3 times. With each cycle, the BCNU concentration was increased from 5 mg/kg, to 7.5 mg/kg, to 10 mg/kg. At week 18 animals were sacrificed for tissue sample analysis and harvest of bone marrow Lin⁻ cells for secondary transplantation into lethally irradiated C57Bl/6 mice, which were then followed for another 16 weeks. (FIG. 104B) Detection of γ-globin expression in peripheral red blood cells by flow cytometry for the “combo” and “cut” groups. (FIG. 104C) γ-globin protein levels measured by HPLC. Right panel: Chromatogram of RBC lysates (week 18) with human β-globin, reactivated human Ay, and added γ-globin chains marked. Left panel: Summary of HPLC data. Shown is the percentage of total γ-globin relative to human β-globin for CD46/β-YAC mice treated with the “cut”, “add”, and “combo” vector. *: p<0.05, n.s. (FIG. 104D) γ-globin mRNA expression relative to mouse β-major mRNA expression (measured by qRT-PCR). (FIG. 104E) Percent target site cleavage by CRISPR/Cas9. Genomic DNA from PBMCs and bone marrow MNCs harvested at week 18 from in vivo “cut” and “combo” transduced mice were subjected to T7E1 assay. Shown is the summary of data from FIG. 105. *p<0.05). (FIG. 104F) Integrated vector copy numbers measured in bone marrow HSPCs at week 18 after transduction with the “add” and “combo” vectors. The difference between the groups is not significant. (FIG. 104G) Spectrum of VCNs in individual CFU's from “combo” vector treated mice. Bone marrow Lin⁻ cells were plated for progenitor assays and VCN was measured in individual colonies by qPCR. Shown are data from four different mice. (FIG. 104H) Human γ/human β globin protein by HPLC.

FIG. 105. Chromatograms of RBC lysates with marked human β- and γ-globin peaks. Upper panel shows β-YAC mice before treatment. Middle panels show week 18 after HDAd-CRISPR (“cut”) transduction. The left panel shows the reactivation of both Gγ and Aγ. Lower panels show week 18 after HDAd-CRISPR (“cut”) transduction. The peaks are labeled in the last bottom panel. Each chromatogram is an individual animal. Note that human β-globin decreases with increased and γ-globin (reverse globin switch).

FIG. 106. T7EI assay data from MNCs from blood, spleen, and bone marrow at week 16 after transduction with “cut” and “combo” vectors. The specific CRISPR/Cas9 cleavage fragments (255 and 110 bp) are marked by arrows. The percentage of cleavage based on band signal quantification is shown below each lane.

FIGS. 107A-107F. Analysis of secondary recipients of Lin⁻ cells from CD46/β-YAC transduced mice. (FIG. 107A) Percentage of human γ-globin expressing peripheral blood RBCs at the indicated time points. All mice received immunosuppression starting from week 4 post-transplantation. (FIG. 107B) Level of γ-globin protein relative to human β-globin at week 16 after transplantation. (FIGS. 107C and 107D) Level of γ-globin protein relative to mouse βₘₐⱼₒᵣ-globin and human β-globin. (FIG. 107E) Lineage-positive cell composition in MNCs of blood, spleen, and bone marrow at week 16 after transduction with the “combo” vector compared to untransduced control mice. FIG. 107F. Vector copy number per cell in total leukocytes from HDAd-combo group measured by qPCR using γ-globin primers.

FIGS. 108A-108D. Generation and characterization of triple transgenic CD46/Townes mice as a model for SCD. (FIG. 108A) Breeding of CD46/Townes mice. Townes mice (hα/hα::β^{S}/β^{S}) were bred over three rounds with CD46 transgenic mice. Animals that were homozygous for CD46, HbS and HBA were used for in vivo transduction studies. (FIG. 108B) Peripheral blood smear of CD46/Townes mice with typical features of the human disease, including anisopoikilocytosis, polychromasia (black arrows), sickled and fragmented cells (black arrows with a star) The scale bar is 15 μm. (FIG. 108C) Hematological analysis of peripheral blood from CD46/Townes mice compared to parental “healthy” CD46-transgenic mice. Ret: reticulocytes; RBC: red blood cells, Hb: hemoglobin; HCT: hematocrit; WBC: white blood cells. All differences are significant (p<0.05). (FIG. 108D) Splenomegaly in CD46/Townes mice. Shown is the ratio of spleen to body weight in CD46tg and CD46/Townes mice. N=3.

FIGS. 109A-109F. γ-globin expression after in vivo HSPC transduction of CD46/Townes mice. Mice were mobilized, HDAd-combo+HDAd-SB injected, and treated with O⁶BG/BCNU as described for FIG. 104. (FIG. 109A) γ-globin marking in peripheral RBCs measured by flow cytometry. The empty squares show marking in RBCs of untreated CD46/Townes mice. The vertical arrows indicate in vivo selection cycles. (FIG. 109B) γ-globin levels in RBCs measured at week 13 by HPLC. Left Panel: Summary of total γ-globin levels relative to human α-globin and β^{S}-globin chains in individual mice. The empty squares show levels in RBCs of untreated CD46/Townes mice. Right panel: Representative chromatograms of CD46/Townes mice before treatment (upper panel) and at week 13 after in vivo HSPC transduction with HDAd-combo+HDAd-SB. The peaks for human β-, β^{S}, reactivated Aγ, and added γ-globin are indicated. (FIG. 109C) Percentage of re-activated Ay based on HPLC. (FIG. 109D) Percentage of total γ-globin mRNA relative to human α-globin and β^{S}-globin mRNA in individual mice. (FIG. 109E) Integrated vector copy numbers measured in bone marrow HSPCs at week 163 after transduction with HDAd-combo. (FIG. 109F) HBG1/2 target site cleavage total bone marrow nuclear cells, Lin⁻ cells, PBMCs, and splenocytes of CD46/Townes mice at week 13 after injection of HDAd-combo. The specific CRISPR/Cas9 cleavage fragments (255 and 110 bp) are marked by arrows. The percentage of cleavage based on band signal quantification is shown below each lane.

FIGS. 110A, 110B. Analysis of secondary recipients transplanted with Lin⁻ cells from transduced CD46/Townes mice. (FIG. 110A) Percentage of human γ-globin expressing peripheral blood RBCs. (FIG. 110B) Level of γ-globin protein relative to human α- and β_{S} globin at week 16 after transplantation.

FIGS. 111A-111C. Phenotypic correction in blood. (FIG. 111A) Blood smears stained for reticulocytes by Brilliant cresyl blue. This dye stains remnants of nuclei and cytoplasmic compartments. (A quantification can be found in FIG. 109C, first group of bars). The scale bar is 20 μm. (FIG. 111B) Blood smears showing the normocytic morphology of erythrocytes after HDAd-combo gene therapy. (FIG. 111C) Hematological analysis of peripheral blood. The differences between “CD46” and “CD46/Townes wk13 after combo” are not significant.

FIGS. 112A-112C. Phenotypic correction in spleen and liver. (FIG. 112A) Tissue histology. Upper panel: iron deposition in spleen. Hemosiderin was detected in spleen sections by Perl's Prussian blue staining. The scale bar is 20 μm. Middle and lower panels: extramedullary hemopoiesis by hematoxylin/eosin staining in spleen and liver sections. Clusters of erythroblasts in the liver and megakaryocytes in the spleen of CD46/Townes mice are indicated by white arrows. The scale bars are 20 μm. Representative images are shown. (FIG. 112B) Spleen size, a measurable characteristic of compensatory hemopoiesis, in treated CD46/Townes mice is comparable to paternal CD46 mice. (FIG. 112C) 4-fold larger magnification of liver section images from FIG. 112A. Sickled RBCs trapped a liver sinusoid of CD46/Townes mice before treatment (left panel) and absence of sickled erythrocytes in liver sinusoids after treatment (right panel).

FIG. 113. The left end of Ad5/35 helper virus genome. The sequences shaded in dark grey correspond to the native Ad5 sequence, i.e., the unshaded or light grey highlighted sequences were artificially introduced. The sequences highlighted in light grey are 2 copies of the (tandemly repeated) loxP sequences. In the presence of “cre recombinase” protein, the nucleotide sequence between the two loxP sequences are deleted (leaving behind one copy of loxP). Because the Ad5 sequence between the loxP sites is essential for packaging the adenoviral DNA into capsids (in the nucleus of the producer cell), this deletion results in the helper adenovirus genome DNA not to be packageable. Consequently, the efficiency of the deletion process has a direct influence of the level of packaged helper genomic DNA (the undesired helper virus “contamination”). In view of the above, in order to translate the same scheme to adenovirus serotypes other than Ad5, it is desirable to achieve the following: 1. Identify the sequences that are essential for packaging, so that they can be flanked by loxP sequence insertions and deleted in the presence of cre recombinase. Identification of these sequences is not straightforward if there is little similarity in sequences. 2. Determine where in the native DNA sequence the insertion of loxP sequence would have the least effect for the propagation and packaging of helper virus (in the absence of cre recombinase). 3. Determine the spacing between the loxP sequences to allow for efficient deletion of packaging sequences and keeping helper virus packaging to a minimum during the production of helper-dependent adenovirus (i.e., in a cre recombinase—expressing cell line such as the 116 cell line).

FIG. 114. Alignment of Ad5 and Ad35 packaging signals (SEQ ID NOs: 49 and 50). The alignment of the left end sequences of Ad5 with Ad35 help in identifying packaging signals. The motifs in the Ad5 sequence that are important for packaging (A1 through AV) are in boxes (see FIG. 1B of Schmid et al., J Virol., 71(5):3375-4, 1997). The location of the loxP insertion sites are indicated by black arrows. It is seen that the insertions flank AI to AIV and disrupt AV. Please note that the additional packaging signal AVI and AVII, as indicated in Schmid et al., have been deleted in the Ad5 helper virus as part of the E1 deletion of this vector.

FIG. 115. Schematic of pAd35GLN-5E4. This is the first-generation (E1/E3-deleted) Ad35 vector derived from a vectorized Ad35 genome (Holden strain from the ATCC) using a recombineering technique (PMID: 28538186). This vector plasmid was then used to insert loxP sites.

FIG. 116. Information on plasmid packaging signals. The packaging site (PS)1 LoxP insertion sites are after nucleotide 178 and 344. This should remove AI to AIV. The rest of the packaging signal including AVI and AVII (after 344) has been deleted (as part of the E1 deletion (345 to 3113)). The PS2 LoxP insertion sites are after nucleotide 178 and 481. Additionally, nucleotides 179 to 365 have been deleted, so AI through AV are not present. The remaining packaging motifs AVI and AVII are removable by cre recombinase during HDAd production. The E1 deletion is from 482 to 3113. The PS3 LoxP insertion sites are after nucleotide 154 and 481. Three engineered vectors could be rescued. The percentage of viral genomes with rearranged loxP sites was 50, 20, and 60% for PS1, PS2, and PS3, respectively. Rearrangements occur when the lox P sites critically affected viral replication and gene expression. Vectors with rearranged loxP sites can be packaged and will contaminate the HDAd prep. SEQ ID NOs: 286, 51, and 52 exemplify the vectors diagramed as PS1, PS2, and PS3, respectively.

FIG. 117. Next generation HDAd35 platform compared to current HDAd5/35 platform. Both vectors contain a CMV-GFP cassette. The Ad35 vector does not contain immunogenic Ad5 capsid protein. Shows comparable transduction efficiency of CD34+ cells in vitro. Bridging study shows comparable transduction efficiency of CD34+ cells in vitro. Human HSCs, peripheral CD34+ cells from G-CSF mobilized donors were transduced with HDAd35 (produced with Ad35 helper P-2) or a chimeric vector containing the Ad5 capsid with fiber from Ad35, at MOIs 500, 1000, 2000 vp/cell. The percentage of GFP-positive cells was measured 48 hours after adding the virus in three independent experiments. Notably, infection with HDAd35 triggered cytopathic effect at 48 hours due to helper virus contamination.

FIG. 118. The PS2 helper vector was remade to focus on monkey studies. The following are actions learned from: deletion of E1 region, a mutant packaging signal flanked by Loxp, mutant packaging sequence, deletion of E3 region (27435430540), replace with Ad5E4orf6, insertion of stuffer DNA flanking copGFP cassette, and introduction of mutation in the knob to make Ad35K++.

FIG. 119. Mutated packaging signal sequence provided. Residues 1 through 137 are the Ad35 ITR. Text in bold are the Swal sites, the Loxp site is italicized, and the mutated packaging signal is underlined.

FIGS. 120A, 120B. Schematic drawings of various helper vector and packaging signal variants. In embodiments, the E3 region (27388→30402) is deleted and the CMV-eGFP cassette is located within an E3 deletion, Ad35K++, and eGFP is used instead of copGFP. All four helper vectors containing the packaging signal variants shown in (FIG. 120A) could be rescued. loxP sites were rearranged as amplification could be more efficient. Additional packaging signal variants are exemplified in FIG. 120B.

FIG. 121. Depiction of a HDAd-combo vector.

FIG. 122. Experimental protocol.

FIG. 123. Vectors for editing the GATAA motif within the +58 erythroid bcl11a enhancer region. The vector structure is shown in the upper panel. Both vectors target the GATAA motif. The lower panel shows the base change mediated by the HDAd-C-BE vector. (SEQ ID NOs: 65-68)

FIGS. 124A-124C. Analysis of vectors on human CD34+ cells. (FIG. 124A) Cell were infected with a MOI of 2000 vp/cell and one day later subjected to erythroid differentiation for 18 days. (FIG. 124B) Cell aliquots were analyzed for target site cleavage by T7E1A assay at different time points. Left bars: HDAd-wtCRISPR, right bars: HDAd-C-BE. (FIG. 124C) Percentage of γ-globin⁺ cells at the end of erythroid differentiation.

FIG. 125. Engraftment of HDAd-wtCRISPR and HDAd-C-BE transduced CD34+ cells. The MOI of transduction was 2000 vp/cell. Engraftment was measured based on the percentage of human CD45+ cells in peripheral blood mononuclear cells.

FIG. 126. Base editor HDAd vectors. The sgRNAs target the erythroid bcl11a enhancer (upper panel) or the BCL11a protein binding site in the HBG1/2. The middle panels show the % of base conversion at the day of erythroid differentiation of erythroid progenitor cells line HUDEP-2. The right panels show the level of γ-globin reactivation. (SEQ ID NOs: 67, 65, and 71)

FIGS. 127A, 127B. (FIG. 127A) Blood smear with typical sickle-like erythrocytes. (FIG. 127B) erythroid parameters.

FIGS. 128A-128C. (FIG. 128A) In vivo transduction of Townes/CD46 mice without in vivo selection. (FIG. 128B) γ-globin reactivation in RBCs. (FIG. 128C) reticulocyte staining of blood smears before and at week 8 of treatment.

FIGS. 129A-129D. In vivo HSC transduction in mobilized macaques. Following mobilization with G-CSF, SCF, and AMD3100, two male macaques received HDAd-GFP (1×10¹²vp/kg) by in intravenous injection. Before HDAd injection, animals were pretreated with dexamethasone to block potential cytokine release. (FIG. 129A) Purified peripheral blood CD34+ cells from the indicated time points were cultured and analyzed for GFP expression by flow cytometry. Shown is the average percent of cells expressing GFP over 4 days in culture (FIG. 129B) Representative flow plots of purified CD34+ cells expressing GFP either before (0 hr) or after (6 hr) HDAd-GFP injection. (FIG. 129C) Colony forming assays were initiated with either purified CD34+ cells from peripheral blood or from total PBMC. After 14 days in culture, individual colonies were picked and analyzed for the presence of GFP DNA by PCR. (FIG. 129D) Analysis of GFP expression in bone marrow CD34+ cells. A representative blot is shown. In this study, only HDAd-GFP was injected and therefore only short-term GFP expression was measured.

FIG. 130. Screening of guide sequences. HUDEP-2 cells were transfected with base editors listed in Table 14. The γ-globin expression was measured at 4 days after transfection (4dpt) and 6 days after in vitro erythroid differentiation (Diff 6d). A CRISPR/Cas9 vector targeting the TGACCA motif in HBG1/2 promoter was used as a positive control (pos ctrl). A CBE targeting CCR5 coding region was included as a negative control (sgNeg). Data shown (mean±SD) are representative of two independent experiments.

FIGS. 131A, 131B. Comparison of different versions of cytidine base editors. (FIG. 131A) 293 cells (HEK293) were transfected were transfected WTCas9 or BE vectors+pSP-BE4-sgBCL11Ae1 (3+1 μg) bcl11a enhancer target site cleavage was analyzed 4 days after transfection by T7E1 assay. (FIG. 131B) The same study was performed in an erythroleukemia cell lines (K562) WTCas9 or BE vectors+pSP-BE4-sgBCL11Ae1 (2+0.66 μg).

FIGS. 132A-132C. Design and rescue of HDAd5/35++_BE vectors. (FIG. 132A) Cytidine base editor (CBE) vector design. Rescuable but low yield. (FIG. 132B) 1st version of adenine base editor (ABE) vector design. Not rescuable. (FIG. 132C) ABE codon optimization to reduce repetitiveness. Includes a sequence comparison showing codon optimization of TadA (tRNA adenosine deaminase enzyme) (SEQ ID NOs: 260 and 261)

FIGS. 133A-133H. Construction and validation of HDAd5/35++BE vectors. (FIG. 133A) HDAd_ABE vector diagram. The 4.2 kb MGMT/GFP cassette flanked by two frt-IRs allows for integrated expression when co-delivered with HDAd_SB vector. The 8.0 kb base editor components were designed outside of the transposon for transient expression. The two TadAN repeats were codon optimized to reduce repetitive sequence (* denotes the catalytic repeat). A microRNA responsive element (miR) was embedded in the 3′ human β-globin UTR to minimize toxicity to producer cells by specifically downregulating ABE expression in 116 cells. PGK, human PGK promoter. bGHpA, bovine growth hormone polyadenylation sequence. SV40 pA, simian virus 40 polyadenylation signal. ITR, inverted terminal repeat. ψ, packaging signal. (FIG. 133B) Information of generated viral vectors. Listed yields are from one 3 L spinner. (FIG. 133C) Validation of viral vectors in HUDEP-2 cells. Cells were transduced with various vectors at indicated MOI (vp/cell). The γ-globin expression was measured at 4 days after transfection (4dpt) and 6 days after in vitro erythroid differentiation (Diff 6d). A CBE vector targeting CCR5 coding region was included as a negative control (sgNeg). Data shown (mean±SD) are representative of two independent experiments. (FIG. 133D) Target base conversion by HDAd_sgHBG #2. HBG1 or HBG2 genomic segments encompassing the targeting bases were amplified and subjected to Sanger sequencing. Data were analyzed by EditR 1.0.9. The arrows indicate targeting bases. The % of conversions were shown below the chromatograms. (FIG. 133E) % of γ-globin expression over α- or β-globin measured by HPLC at day 6 after differentiation. MOI=1000. Data shown (mean±SD) are representative of two independent experiments. FIGS. 133F-133H) A representative clone (#3) derived from HUDEP-2 cells transduced with HDAd_sgHBG #2. Monoallelic-116A→G base conversion was detected in HBG1 promoter (FIG. 133F), resulting in 100% γ-globin⁺ cells by flow cytometry (FIG. 133G). The γ-globin protein level was measured by HPLC (FIG. 133H).

FIGS. 134A-134C. Data supporting FIG. 133. (FIG. 134A) Supplementary to FIG. 133D. Target base conversion in HUDEP-2 cells treated with indicated viruses. (FIG. 134B) Representative single cell HUDEP-2 clones. Supplementary to FIG. 133F. The B with an arrow indicates biallelic editing and the M and arrow indicates the monoallelic editing. (FIG. 134C) γ-globin expression in corresponding single cell HUDEP-2 clones shown above. Supplementary to FIG. 133G.

FIGS. 135A-1351. Reactivation of γ-globin in YAC mice after in vivo transduction and selection. (FIG. 135A) Experiment procedure. β-YAC/CD46 mice (n=9) were mobilized by G-CSF/AMD3100 and in vivo transduced with HDAd_sgHBG #2+HDAd_SB. Four rounds of selection by O⁶BG/BCNU were performed at week 4, 6, 8 and 10 weeks after transduction, respectively. The mice were euthanized at week 16. The lineage⁻ cells were isolated and IV injected into lethally irradiated C57BL/6 mice. The secondary transplanted mice were followed for another 16 weeks. (FIG. 135B) GFP marking in PBMCs at various time points after transduction. Each dot represents one animal. (FIG. 135C) Representative dot plots of GFP expression in PBMCs. (FIG. 135D) γ-globin expression in blood cells measured by flow cytometry. (FIG. 135E) Representative dot plots of γ-globin expression in blood cells. (FIG. 135F) γ-globin expression by flow cytometry in Ter-119+ and Ter-119⁻ cells in blood and bone marrow at terminal point in primary mice. (FIG. 135G) γ-globin protein level in red blood cell lysates measured by HPLC. Data shown are percentage over mouse α- or β-globin or human β-globin. (FIG. 135H) γ-globin expression at mRNA level measured by RT-PCR. Data shown are fold of change over mouse HBA or HBB, or human HBB mRNA. (FIG. 135I) Vector copy number (copies per cell) in total bone marrow cells. Primers to MGMT were used.

FIG. 136. HPLC plot of representative data shown in FIG. 135H.

FIGS. 137A-137G. Target base conversion. (FIG. 137A) sgHBG #2 guide sequence. The numbering was started from 5′ end. Highlighted with orange background is TGACCA motif, a reported BCL11A binding site. The two adenines (A5 and A8) in the motif was indicated by the two arrows. (FIG. 137B) Percentage of target base conversion. Both A5 and A8 in HBG1 and HBG2 promoter regions were shown. Each dot represents one animal (n=9). (FIG. 137C) Representative chromatograms showing target base conversion in HBG1 and HBG2 regions of mouse #1108. (FIG. 137D) Correlation between average base conversion versus γ-globin expression. The percentage of average base conversion in each animal was the average level at A5 and A8 in HBG1 and HBG2 promoter regions. Each dot represents one animal (n=9). (FIG. 137E) Comparison of base conversion at A5 and A8. Each dot represents one animal (n=9). (FIG. 137F) Chart showing percentage of conversion at targeted adenine nucleotides. (FIG. 137G) Chromatogram showing targeting base conversion in a particular mouse (SEQ ID NO: 250).

FIGS. 138A-138D. Safety profile. (FIG. 138A) Hematology analysis by HEMAVET® using blood samples at week 16 after transduction. Data shown are mean±SD representing 9 mice transduced with HDAd_sgHBG #2 and 3 untransduced control mice. (FIG. 138B) Percentage of reticulocytes in blood samples at week 16. The samples were stained by Brilliant cresyl blue. Data shown are mean±SD representing 4 mice transduced with HDAd_sgHBG #2 and 3 untransduced control mice. (FIG. 138C) Cellular composition in bone marrow MNCs at the terminal point of primary mice. Untransduced mice was used as control. Each dot represents one animal. (FIG. 138D) Representative reticulocytes staining by Brilliant cresyl blue.

FIGS. 139A-139C. Secondary transplantation. (FIG. 139A) Engraftment measured by human CD46 expression in PBMCs using flow cytometry. (FIG. 139B) GFP expression in PBMCs. (FIG. 139C) γ-globin expression in peripheral blood cells detected by flow cytometry.

FIGS. 140A, 140B. Detection of intergenic deletion. (FIG. 140A) The detection of intergenic 4.9 k deletion was described previously (Li et al, Blood, 131(26): 2915, 2018). Genomic DNA isolated from total bone marrow MNCs were used as template. A 9.9 kb genomic region spanning the two CRISPR cutting sites at HBG1 and HBG2 promoters was amplified by PCR. An extra 5.0 kb band in the product indicates the occurrence of the 4.9 k deletion. The percentage of deletion was calculated according to a standard curve formula which was generated by PCR using templates with defined ratios of the 4.9 kb deletion. Samples derived from mice in vivo transduced with a CRISPR vector targeting HBG1/2 promoter were used in comparison. Each lane represents one animal. (FIG. 140B) Summary of the percentage of deletion in FIG. 140A. Each dot represents one animal.

FIG. 141. Cytotoxicity of BEs vs CRISPR/Cas9. A major concern with current genome-editing technologies using CRISPR/Cas9 is that they introduce double-stranded DNA breaks (DSBs), which may be detrimental to host cells by causing unwanted large fragment deletion and p53-dependent DNA damage responses. Base editors are capable of installing precise nucleotide mutations at targeted genomic loci and present the advantage of avoiding DSBs. This study shows that a critical functional feature of HSC, namely the engraftment in sub-lethally irradiated NSG mice, is not affect by a BE but is dramatically reduced after transduction of human CD34+ cells with CRISPR/Cas9 expressing vector.

FIG. 142. Expected editing mediated by BE4-sgBCL11AE1. Schematic showing editing of a BCL11A locus. The GATAA motif (SEQ ID NO: 65) and disrupted GATAA motif after base editing (SEQ ID NO: 67) are shown.

FIG. 143. Optimal location for targets. Schematic of a nucleic acid sequence that highlights exemplary locations for targeting. The figure shows, in part, C to T editing when the target C is in positions 4 through 8 within a protospacer.

FIG. 144 is a schematic of a vector encoding a base editor.

FIG. 145. Diagram of viral gDNA. Schematic of a viral gDNA (HBG2-miR, adenine editor) which represents a single contiguous construct but has been divided into two sections solely for ease of presentation.

FIG. 146. TadA sequences. Schematic representations of sequences of TadA and TadA* (SEQ ID NOs: 265 and 266), including DNA sequences of two TadA+32aa′ (SEQ ID NOs: 367 and 268).

FIG. 147. Base editing. Schematic representations of wild type (SEQ ID NO: 269) and edited sequences (SEQ ID NO: 269).

FIG. 148. Base editing. Schematic representation and two gels relating to base editing by an HDAd5/35++_BE4-sgBCL11Ae1-Fl-mgmtGFP (041318-1) virus.

FIG. 149. Percent of γ-globin⁺ cells. Graph showing the percentage of γ-globin⁺ cells at indicated MOIs.

FIG. 150. Reactivation of HbF by base editing. Listing of vectors and related information.

FIG. 151. Listing of vectors and related information, and a graph showing percent HbF+ cells at various MOIs of the base editors.

FIG. 152. γ-globin expression (HUDEP-2), 2nd trial. Graph showing % HbF+from a second trial in HUDEP-2 cells.

FIG. 153. γ-globin expression (HUDEP-2), single cell derived clones. Graph showing the % HbF+ in various single cell derived clones.

FIGS. 154A-154S. Data representing individual single-cell derived clones. Each of FIGS. 154A-154S includes data representative of a single cell clone. (SEQ ID NOs: 271, 250, 252)

FIG. 155. Test in 293FT cells. Two gels showing results of use of base editors in 293FT cells.

FIGS. 156A-156D. Sanger sequencing to confirm edited bases (293FT cells). Each of FIGS. 156A-156D includes chromatogram(s) showing sanger sequencing results. (SEQ ID NOs: 269, 275-278)

FIG. 157. Test in HUDEP-2 cells. Two gels showing results of use of base editors in HUDEP-2 cells 4 days post transfection.

FIG. 158. γ-globin expression (HUDEP-2). Graph showing expression of γ-globin.

FIGS. 159A-159D. Sanger sequencing to confirm edited bases (HUDEP-2 cells). Each of FIGS. 159A-159D includes chromatogram(s) showing Sanger sequencing results, where available. (SEQ ID NOs: 269, 275-278)

FIG. 160. Selected constructs for HDAd virus production (under Maxi preparation). List of constructed vectors indication selection of certain constructs for HDAd virus production (under Maxi preparation).

FIG. 161. Chart showing engraftment of huCD45+ cells.

FIG. 162. Transient transfection of HUDEP-2 cells (cleavage by T7E1). Gels showing results of transient transfection of HUDEP-2 cells (cleavage by T7E1).

FIG. 163. Dual base editing vector application. Schematic representation of a dual base editing vector embodiment (SEQ ID NO: 279).

FIG. 164. Vector schematic of HDad5/35++combo vector showing human γ-globin/mgmt. gene addition by SB100x transposase and rhesus γ-globin re-activation using CRISPRs targeting the erythroid bcl11a enhancer and the BCL11A binding site in the HBG promoter.

FIG. 165. Vector schematic showing HDAd-sgAAVS1-rm (no Cas9) vector and HDAd-Comb2. The properties of this vector are 1.8 k homology arm (HA), GFP for tracking transduction in PBMCs, CRISPR cassette outside HA, and targeting HBG promoter.

FIG. 166. Vector schematic of HDAd-rh-combo with the expression of rh γ-globin using LCR β-globin promoter driven exogenous γ-globin and reactivation of endogenous γ-globin via CRISPR/Cas9-mediated disruption of repressor binding region of γ-globin promoter.

### DETAILED DESCRIPTION

The current disclosure describes, among other things, recombinant adenoviral vectors, such as Ad5/35 and Ad35 vectors targeting CD46 for in vivo gene editing of hematopoietic stem cells. Ad35 vectors can include knob protein mutations that increase CD46 binding, miRNA control systems that regulate expression of genes, CRISPR components to activate endogenous gene expression, positive selection markers, mini- or long-form β-globin locus control regions (LCR) regulatory sequences, transposase/recombinase systems, and/or various other sequences disclosed herein, including without limitation a number of other beneficial advances that promote conditioning-free in vivo gene therapies.

Despite the development of many tools for gene therapy, design of vectors and/or therapeutically useful payloads remains an important challenge in the field. Gene therapy payloads can be delivered by viral vectors or non-viral vectors. Exemplary non-viral vectors include cationic lipid, lipid nano emulsion, solid lipid nanoparticle, peptide, and polymer-based delivery systems. Viral vectors can include AAV, herpes simplex, retroviral, lentivirus, alphavirus, flavivirus, rhabdovirus, measles virus, Newcastle disease virus, poxvirus, picornavirus, coxsackievirus vectors, and adenovirus vectors, each with various distinct characteristics. Among adenoviruses, there are also over 50 serotypes. Therapeutic payloads for expression and/or modification of nucleic acid sequences also exist, including without limitation payloads encoding proteins, regulatory nucleic acids, CRISPR/Cas9 systems, base editing systems, transposon systems, and homologous recombination systems. Methods and compositions for gene therapy provided herein address, without limitation, various challenges in the utilization of adenoviral vectors and/or various therapeutic payloads.

While disclosure in the present specification may be in a particular context (e.g., an adenoviral vector or genome context, e.g., an Ad5, Ad5/35, or Ad35 context), each component is further disclosed independent of any such context and as such may be claimed independently of such context. Exemplary disclosures include sequences and payload constructs of the present disclosure, which those of skill in the art will appreciate can have general relevance not limited to any particular vector, serotype, or other context.

Aspects of the current disclosure are now described in additional detail as follows: (I) Gene Therapy Vectors; (II) Target Cell Populations; (III) Dosages, Formulations, and Administration; (IV) Applications; (V) Exemplary Embodiments; (VI) Experimental Examples; and (VII) Closing Paragraphs.

### I. GENE THERAPY VECTORS

Adenovirus (or, interchangeably, “adenoviral”) vectors and genomes refer to those constructs containing adenovirus sequences sufficient to (a) support packaging of an expression construct and to (b) express a coding sequence. Adenoviral genomes can be linear, double-stranded DNA molecules. As those of skill in the art will appreciate, a linear genome such as an adenoviral genome can be present in circular plasmid, e.g., for viral production purposes.

Natural adenoviral genomes range from 26 kb to 45 kb in length, depending on the serotype.

Adenoviral vectors include Adenoviral DNA flanked on both ends by inverted terminal repeats (ITRs), which act as a self-primer to promote primase-independent DNA synthesis and to facilitate integration into the host genome. Adenoviral genomes also contain a packaging sequence, which facilities proper viral transcript packaging and is located on the left arm of the genome. Viral transcripts encode several proteins including early transcriptional units, E1, E2, E3, and E4 and late transcriptional units which encode structural components of the Ad virion (Lee et al., Genes Dis., 4(2):43-63, 2017).

Adenoviral vectors include adenoviral genomes. Recombinant adenoviral vectors are adenoviral vectors that include a recombinant adenoviral genome. A recombinant adenoviral vector includes a genetically engineered form of an adenovirus. Those of skill in the art will appreciate that throughout the present application disclosure of an adenoviral vector includes disclosure of the adenoviral genome thereof, and that disclosure of an adenoviral genome includes disclosure of an adenoviral vector including the disclosed adenoviral genome.

The adenovirus is a large, icosahedral-shaped, non-enveloped virus. The viral capsid includes three types of proteins including fiber, penton, and hexon based proteins. The hexon makes up the majority of the viral capsid, forming the 20 triangular faces. The penton base is located at the 12 vertices of the capsid and the fiber (also referred to as knobbed fiber) protrudes from each penton base. These proteins, the penton and fiber, are of particular importance in receptor binding and internalization as they facilitate the attachment of the capsid to a host cell (Lee et al., Genes Dis., 4(2):43-63, 2017).

Ad35 fiber is a fiber protein trimer, each fiber protein including an N-terminal tail domain that interacts with the pentameric penton base, a C-terminal globular knob domain (fiber knob) that functions as the attachment site for the host cell receptors, and a central shaft domain that connects the tail and the knob domains (shaft). The tail domain of the trimeric fiber attaches to the pentameric penton base at the 5-fold axis. In various embodiments, an Ad35 fiber knob includes amino acids 123 to 320 of a canonical wild-type Ad35 fiber protein. In various embodiments, an Ad35 fiber knob includes at least 60 amino acids (e.g., at least 60, 70, 80, 90, 100, 110, 120, 130, 140, 150, 160, 170, 180, 190, or 198 amino acids) having at least 80% (e.g., at least 80%, 85%, 90%, 95%, 96%, 97%, 98%, or 99% sequence identity) sequence identity with a corresponding fragment of amino acids 123 to 320 of a canonical wild-type Ad35 fiber protein. In various embodiments, a fiber knob is engineered for increased affinity with CD46, and/or to confer increased affinity with CD46 to a fiber protein, fiber, or vector, as compared to a reference fiber knob, fiber protein, fiber or vector including a canonical wild-type Ad35 fiber protein, optionally wherein the increase is an increase of at least 1.1-fold, e.g., at least 1, 2, 3, 4, 5, 10, 15, or 20-fold. The central shaft domain consists of 5.5 β-repeats, each containing 15-20 amino acids that code for two anti-parallel β-strands connected by a β-turn. The 3-repeats connect to form an elongated structure of three intertwined spiraling strands that is highly rigid and stable.

Adenovirus is particularly suitable for use as a gene transfer vector because of its mid-sized genome, ease of manipulation, high titer, wide target-cell range and high infectivity. Both ends of the viral genome contain 100-200 base pair ITRs, which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5′-tripartite leader (TPL) sequence which makes them preferred mRNAs for translation.

### I(A). Gene Therapy Vector Serotypes

Among adenoviruses, there are also over 50 serotypes. Adenovirus type 5 is a human adenovirus about which a great deal of biochemical and genetic information is known, and it has historically been used for most constructions employing adenovirus as a vector. Ad5 has been widely used in gene therapy research.

The majority of humans, however, have neutralizing serum antibodies directed against Ad5 capsid proteins, which can block in vivo transduction with adenoviral vectors that include an Ad5 capsid, such as HDAd5/35 vectors, i.e. vectors that contain Ad5 capsid proteins and chimeric Ad35 fibers. While the existence of neutralizing serum antibodies directed against Ad5 capsid proteins does not negate the therapeutic value of adenoviral vectors that include Ad5 capsids, adenoviral vectors that do not include Ad5 capsids would provide an additional benefit in that the general risk of a clinically significant immunogenic response would be reduced, particularly in subjects that have neutralizing serum antibodies directed against Ad5 capsid proteins.

Ad35 is one of the rarest of the 57 known human serotypes, with a seroprevalence of <7% and no cross-reactivity with Ad5. Ad35 is less immunogenic than Ad5, which is, in part, due to attenuation of T-cell activation by the Ad35 fiber knob. Further, after intravenous (iv) injection, there is only minimal transduction (only detectable by PCR) of tissues, including the liver, in human CD46 transgenic (hCD46tg) mice and non-human primates. First-generation Ad35 vectors have been used clinically for vaccination purposes.

### I(A)(i). Ad35 Gene Therapy Vectors

The complete genome of a representative natural Ad35 adenovirus is known and publicly available (see, e.g., Gao et al., 2003 Gene Ther. 10(23): 1941-9; Reddy et al. 2003 Virology 311(2): 384-393; GenBank Accession No. AX049983). While the Ad5 genome is 35,935 bp with a G+C content of 55.2%, the Ad35 genome is 34,794 bp with a G+C content of 48.9%. The genome of Ad35 is flanked by inverted terminal repeats (ITRs). In various embodiments, Ad35 ITRS include 137 bp (e.g., a 5′ Ad35 that includes nucleotides 1-137 or 4-140 of GenBank Accession No. AX049983 and a 3′ ITR that includes nucleotides 34658-34794 of GenBank Accession No. AX049983), which are longer than those of Ad5 (103 bp). In various embodiments, an Ad35 5′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides, e.g., 137 nucleotides) having at least 80% sequence identity (e.g., at least 80%, 85%, 90%, 95%, 96%, 97%, 98%, or 99% A sequence identity) with a corresponding fragment of nucleotides 1-200 of GenBank Accession No. AX049983 and an Ad35 3′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides, e.g., 137 nucleotides) having at least 80% sequence identity (e.g., at least 80%, 85%, 90%, 95%, 96%, 97%, 98%, or 99% sequence identity) with a corresponding fragment of nucleotides 34595-34794 of GenBank Accession No. AX049983. In various embodiments, an ITR is sufficient for one or both of Ad35 encapsidation and/or replication. In various embodiments, an Ad35 ITR sequence for Ad35 vectors differs in that the first 8 bp are CTATCTAT rather than CATCATCA (Wunderlich, J. Gen Viro. 95: 1574-1584, 2014).

In various embodiments, packaging of the adenovirus genome is mediated by a cis-acting packaging sequence domain located at the 5′ end of the viral genome adjacent to the ITR, and packaging occurs in a polar fashion from left to right. The packaging sequence of Ad35 is located at the left end of the genome with five to seven putative “A” repeats. In various embodiments, the present disclosure includes a recombinant Ad35 donor vector or genome that includes an Ad35 packaging sequence. In various embodiments, the present disclosure includes a recombinant Ad35 helper vector or genome that includes a packaging sequence flanked by recombinase sites. In various embodiments, an Ad35 packaging sequence refers to a nucleic acid sequence including nucleotides 138-481 of GenBank Accession No. AX049983 or a fragment thereof sufficient for or required for packaging of an Ad35 vector or genome (e.g., such that flanking of the sequence with recombinase sites and excision by recombination of the recombinase sites renders the vector or genome deficient for packaging, e.g., by at least 10% as compared to a reference including the packaging sequence, e.g., by at least 10%, 20%, 30%, 40$, 50%, 60%, 70%, 80%, 90%, 91%, 92%, 93%, 94%, 95%, 96%, 97%, 98%, 99%, or 100%, optionally wherein the reference includes the packaging sequence flanked by the recombines sites). In various embodiments, an Ad35 packaging sequence includes at least 80 nucleotides (e.g., at least 80, 90, 100, 110, 120, 130, 140, 150, 160, 170, 180, 190, 200, 225, 250, 275, or 300 nucleotides, e.g., a number of nucleotides having a lower bound of 80, 90, 100, 110, 120, 130, 140, or 150 nucleotides and an upper bound of 150, 160, 170, 180, 190, 200, 225, 250, 275, or 300 nucleotides) having at least 80% sequence identity (e.g., at least 80%, 85%, 90%, 95%, 96%, 97%, 98%, or 99% sequence identity) with a corresponding fragment of nucleotides 137-481 of GenBank Accession No. AX049983.

In various embodiments, an Ad35 helper vector can include recombinase sites inserted to flank a packaging sequence, where a first recombinase site is inserted immediately adjacent to (e.g., before, or after) a position selected from between nucleotide 130 and nucleotide 400 (e.g., between nucleotides 138 and 180, 138 and 200, 138 and 220, 138 and 240, 138 and 260, 138 and 280, 138 and 300, 138 and 320, 138 and 340, 138 and 360, 138 and 366, 138 and 380, or 138 and 400) and a second recombinase site inserted immediately adjacent to (e.g., after, or before) a position selected from between nucleotide 300 and nucleotide 550 (e.g., between nucleotides 344 and 360, 344 and 380, 344 and 400, 344 and 420, 344 and 440, 344 and 460, 344 and 480, 344 and 481, 344 and 500, 344 and 520, 344 and 540, or 344 and 550). Those of skill in the art will appreciate that the term packaging sequence does not necessarily include all of the packaging elements present in a given vector or genome. For example, a helper genome can include recombinase direct repeats that flank a packaging sequence, where the flanked packaging sequence does not include all of the packaging elements present in the helper genome. Accordingly, in certain embodiments, one or two recombinase direct repeats of a helper genome are positioned within a larger packaging sequence, e.g., such that a larger packaging sequence is rendered noncontiguous by introduction of the one or two recombinase direct repeats. In various embodiments, recombinase direct repeats of a helper genome flank a fragment of the packaging sequence such that excision of the flanked packaging sequence by recombination of the recombinase direct repeats reduces or eliminates (more generally, disrupts) packaging of the helper genome and/or ability of the helper genome to be packaged. By way of example, recombinase direct repeats (DRs) are positioned within 550 nucleotides of the 5′ end of the Ad35 genome in order to functionally disrupt the Ad35 packaging signal but not the 5′ Ad35 ITR. In various embodiments, the DRs are positioned closer than 550 nucleotides from the 5′ end of the Ad35 genome, for instance within 540, 530, 520, 510, 500, 495,490, 480, 470, 450, 440, 400, 380, 360 nucleotides, or closer than within 360 nucleotides of the 5′ end of the Ad35 genome, in order to functionally disrupt the Ad35 packaging signal but not the 5′ Ad35 ITR.

In various embodiments, the present disclosure includes a recombinant Ad35 donor vector or genome that includes an Ad35 5′ ITR, an Ad35 packaging sequence, and an Ad35 3′ ITR, In certain embodiments, an Ad35 5′ ITR, an Ad35 packaging sequence, and an Ad35 3′ ITR are the only fragments of the recombinant Ad35 donor vector or genome (e.g., the only fragments over 50 or over 100 base pairs) that are derived from, and/or have at least 80% identity to, a canonical Ad35 genome.

Ad35 early regions include E1A, E1B, E2A, E2B, E3, and E4. Ad35 intermediate regions include pIX and IVa2. The late transcription unit of Ad35 is transcribed from the major late promoter (MLP), located at 16.9 map units. The late mRNAs in Ad35 can be divided into five families of mRNAs (L1-L5), depending on which poly(A) signal is used by these mRNAs. Based on the MLP consensus initiator element, and splice donor and splice acceptor site sequences, the length of tripartite leader (TPL) has been predicted to be 204 nucleotides. The first leader of the TPL, which is adjacent to MLP, is 45 nucleotides in length. The second leader located within the coding region of DNA polymerase is 72 nucleotides in length. The third leader lies within the coding region of precursor terminal protein (pTP) of E2B region and is 87 nucleotides in length. While Ad5 contains two virus-associated (VA) RNA genes, only one virus-associated RNA gene occurs in the genome of Ad35. This VA RNA gene is located between the genes coding for the 52/55K L1 protein and pTP.

In particular embodiments, an Ad35++ vector is a chimeric vector with a mutant Ad35 fiber knob (e.g., a recombinant Ad35 vector with a mutant Ad35 fiber knob or an Ad5/35 vector with a mutant Ad35 fiber knob). In particular embodiments, an Ad35++ genome is a genome that encodes a mutant Ad35 fiber knob (e.g., a recombinant Ad35 helper genome encoding a mutant Ad35 fiber knob or an Ad5/35 helper genome encoding a mutant Ad35 fiber knob). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593(24): 3623-3648, 2019).

In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank accession AP_000601 or an amino acid sequence corresponding thereto, e.g., where position 207 is Glu or Asp. In various embodiments, an Ad35 fiber has an amino acid sequence according to GenBank accession AP_000601. Further description of Ad35++fiber knob mutations is found in Wang 2008 J. Virol. 82(21): 10567-10579, which is incorporated herein by reference in its entirety and with respect to fiber knobs.

### I(A)(ii). Ad5/35 Gene Therapy Vectors

Ad5/35 vectors of the present disclosure include adenoviral vectors that include Ad5 capsid polynucleotides and chimeric fiber polynucleotides including an Ad35 fiber knob, the chimeric fiber polynucleotide typically also including an Ad35 fiber shaft (e.g., Ad5 fiber amino acids 1-44 in combination with Ad35 fiber amino acids 44-323). In various embodiments, the fiber includes an Ad35++ mutant fiber knob. In various Ad5/35 vectors of the present disclosure, all proteins except fiber knob domains and shaft were derived from serotype 5, while fiber knob domains and shafts were derived from serotype 35, and mutations that increased the affinity to CD46 were introduced into the Ad35 fiber knob (see WO 2010/120541 A2). Additionally, in various embodiments, the ITR and packaging sequence of the Ad5/35 vectors are derived from Ad5. (See Table 1 for exemplary knob mutations; and FIG. 95 for a general schematic of HDAd35 vector production.)

TABLE 1
Mutated Ad35 Knob increased binding to CD46

| | Kd (Oleks) | |
|---|---|---|
| A1: Asn217Asp Thr245Pro | A1 4.82 nM | Asp207Gly +++ |
| Ile256Leu* | | |
| A2: Asp207Gly Thr245Ala* | A2 0.629 nM | Thr245Ala ++ |
| A3: Asp207Gly Thr226Ala* | A3 1.407 nM | Ile256Leu + |
| A8: Ile192Val Ile256Val ? | A8 13.6850 nM | |
| B1: Asp207Gly* | B1 1.774 nM | |
| B2: wtAd35(207Asp) | B2 14.98 nM | |
| B3: Asn217Asp* | B3 16.85 nM | |
| B4: Thr245Ala* | B4 7.64 nM | |
| B5: Ile256Leu* | B5 10.96 nM | |
| B6: Ad3 | B6 no binding | |
| B7: Ad11 | B7 11.22 nM | |
| M1: Arg279Cys* | M1 no binding | |
| M3: Arg279His* | M3 no binding | |
| wtAd35* | 13.7 nM | |
| wtAd35* | 15.36 nM | |
| AA: Asp207Gly Thr245Ala | 0.943 nM | |
| Ile256Leu* | | |

*Published in Wang et al. (J. Virol., 82(21): 10567-10579, 2008)
**Published in Wang et al. (J. Virol. 81 (23): 12785-12792, 2007)

### I(B). Helper-Dependent Ad35 and Ad5/35 Vectors

In general, the path from a natural adenoviral vector to a helper-dependent adenoviral vector can include three generations. First-generation adenoviral vectors are engineered to remove genes E1 and E3. Without these genes, adenoviral vectors cannot replicate on their own but can be produced in E1-expressing mammalian cell lines such as HEK293 cells. With only first-generation modifications, adenoviral vector cloning capacity is limited, and host immune response against the vector can be problematic for effective payload expression. Second-generation adenoviral vectors, in addition to E1/E3 removal, are engineered to remove non-structural genes E2 and E4, resulting in increased capacity and reduced immunogenicity. Third-generation adenoviral vector (also referred to as gutless, high capacity adenoviral vector, or helper-dependent adenoviral vector (HdAd)) are further engineered to remove all viral coding sequences, and retain only the ITRs of the genome and packaging sequence of the genome or a functional fragment thereof. Because these genomes do not encode the proteins necessary for viral production, they are helper-dependent: a helper-dependent genome can only be packaged into vector if they are present in a cell that includes a nucleic acid sequence that provides viral proteins in trans. These helper-dependent vectors are also characterized by still greater capacity and further decreased immunogenicity. Because the sequences of each viral genome are distinct at least for each serotype, the proper modifications required to produce a helper-dependent viral genome, and/or a helper genome, for a given serotype cannot be predicted from available information relating to other serotypes.

Helper-dependent adenoviral vectors (HDAd) engineered to lack all viral coding sequences can efficiently transduce a wide variety of cell types, and can mediate long-term transgene expression with negligible chronic toxicity. By deleting the viral coding sequences and leaving only the cis-acting elements necessary for genome replication (ITRs) and encapsidation (γ), cellular immune response against the Ad vector is reduced. HDAd vectors have a large cloning capacity of up to 37 kb, allowing for the delivery of large payloads. These payloads can include large therapeutic genes or even multiple transgenes and large regulatory components to enhance, prolong, and regulate transgene expression. Like other adenoviral vectors, typical HDAd genome generally remain episomal and do not integrate with a host genome (Rosewell et al., J Genet Syndr Gene Ther. Suppl 5:001, 2011, doi: 10.4172/2157-7412.s5-001).

In some HDAd vector systems, one viral genome (a helper genome) encodes all of the proteins required for replication but has a conditional defect in the packaging sequence, making it less likely to be packaged into a virion. As noted above, this can require identification of the packaging sequence or a functionally contributing (e.g., functionally required) fragment thereof and modification of the subject genome in a manner that does not negate propagation of the helper vector, which cannot be ascertained from existing knowledge relating to other adenoviral serotypes, A separate donor viral genome includes (e.g., only includes) viral ITRs, a payload (e.g., a therapeutic payload), and a functional packaging sequence (e.g., normal wild-type packaging sequence, or a functional fragment thereof), which allows this donor viral genome to be selectively packaged into HDAd viral vectors and isolated from the producer cells. HDAd donor vectors can be further purified from helper vectors by physical means. In general, some contamination of helper vectors and/or helper genomes in HDAd viral vectors and HDAd viral vector formulations can occur and can be tolerated.

In some HDAd vector systems, a helper genome utilizes a Cre/loxP system. In certain such HDAd vector systems, the HDAd donor genome includes 500 bp of noncoding adenoviral DNA that includes the adenoviral ITRs which are required for genome replication, and ψ which is the packaging sequence or a functional fragment thereof required for encapsidation of the genome into the capsid. It has also been observed that the HDAd donor vector genome can be most efficiently packaged when it has a total length of 27.7 kb to 37 kb, which length can be composed, e.g., of a therapeutic payload and/or a “stuffer” sequence. The HDAd donor genome can be delivered to cells, such as 293 cells (HEK293) that expresses Cre recombinase, optionally where the HDAd donor genome is delivered to the cells in a non-viral vector form, such as a bacterial plasmid form (e.g., where the HDAd donor genome is constructed as a bacterial plasmid (pHDAd) and is liberated by restriction enzyme digestion). The same cells can be transduced with the helper genome, which can include an E1-deleted Ad vector bearing a packaging sequence or functionally contributing (e.g., functionally required) fragment thereof flanked by loxP sites so that following infection of 293 cells expressing Cre recombinase, the packaging sequence or functionally contributing (e.g., functionally required) fragment thereof is excised from the helper genome by Cre-mediated site-specific recombination between the loxP sites. Thus, the HDAd donor genome can be transfected into 293 cells (HEK293) that express Cre and are transduced with a helper genome bearing a packaging sequence (γ) or a functional fragment thereof flanked by recombinase sites (e.g., loxP sites) such that excision mediated by a corresponding recombinase (e.g., Cre-mediated excision) of ψ renders the helper virus genome unpackageable, but still able to provide all of the necessary trans-acting factors for propagation of the HDAd. After excision of the packaging sequence or functionally contributing (e.g., functionally required) fragment thereof, a helper genome is unpackageable but still able to undergo DNA replication and thus trans-complement the replication and encapsidation of the HDAd donor genome. In some embodiments, to prevent generation of replication competent Ad (RCA; E1⁺) as a consequence of homologous recombination between the helper and HDAd donor genomes present in 293 cells (HEK293) a “stuffer” sequence can be inserted into the E3 region to render any E1⁺ recombinants too large to be packaged. Similar HDAd production systems have been developed using FLP (e.g., FLPe)/frt site-specific recombination, where FLP-mediated recombination between frt sites flanking the packaging sequence of the helper genome selects against encapsidation of helper genomes in 293 cells (HEK293) that express FLP. Alternative strategies to select against the helper vectors have been developed. An Ad35 helper virus typically includes all of the viral genes except for those in E1, as E1 expression products can be supplied by complementary expression from the genome of a producer cell line.

HDAd5/35 donor vectors, donor genomes, helper vectors and helper genomes are exemplary of compositions provided herein and used in various methods of the present disclosure. An HDAd5/35 vector or genome is a helper-dependent chimeric Ad5/35 vector or genome with an Ad35 fiber knob and an Ad5 shaft. An HDAd5/35++ vector or genome is a helper-dependent chimeric Ad5/35 vector or genome with a mutant Ad35 fiber knob. The vector is mutated to increase the affinity to CD46, e.g., by 25-fold and increases cell transduction efficiency at lower multiplicity of infection (MOI) (Li & Lieber, FEBS Letters, 593(24): 3623-3648, 2019). An Ad5/35 helper vector is a vector that includes a helper genome that includes a conditionally expressed (e.g., frt-site or loxP-site flanked) packaging sequence and encodes all of the necessary trans-acting factors for production of Ad5/35 virions into which the donor genome can be packaged.

HDAd35 donor vectors, donor genomes, helper vectors and helper genomes are also exemplary of compositions provided herein and used in various methods of the present disclosure. An HDAd35 vector or genome is a helper-dependent Ad35 vector or genome. An HDAd35++ vector or genome is a helper-dependent Ad35 vector or genome with a mutant Ad35 fiber knob which enhances its affinity to CD46 and increases cell transduction efficiency. An Ad35 helper vector is a vector that includes a helper genome that includes a conditionally expressed (e.g., frt-site or loxP-site flanked) packaging sequence and encodes all of the necessary trans-acting factors for production of Ad35 virions into which the donor genome can be packaged. The present disclosure further includes an HDAd35 donor vector production system including a cell including an HDAd35 donor genome and an Ad35 helper genome. In certain such cells, viral proteins encoded and expressed by the helper genome can be utilized in production of HDAd35 donor vectors in which the HDAd35 donor genome is packaged. Accordingly, the present disclosure includes methods of production of HDAd35 donor vectors by culturing cells that include an HDAd35 donor genome and an Ad35 helper genome. In some embodiments the cells encode and express a recombinase that corresponds to recombinase direct repeats that flank a packaging sequence of the Ad35 helper vector. In some embodiments, the flanked packaging sequence of the Ad35 helper genome has been excised.

In some embodiments the Ad35 helper genome encodes all Ad35 coding sequences. In some embodiments the Ad35 helper genome encodes and/or expresses all Ad35 coding sequences except for one or more coding sequences of the E1 region and/or an E3 coding sequence and/or an E4 coding sequence. In various embodiments, a helper genome that does not encode and/or express an Ad35 E1 gene does not encode and/or express an Ad35 E4 gene, optionally wherein the Ad35 helper genome is further engineered to include an Ad5 E4orf6 coding sequence. In various embodiments, as will be appreciate by those of skill in the art, cells of compositions and methods for production of HDAd 35 donor vectors can be cells that express an Ad5 E1 expression product. In various embodiments, as will be appreciate by those of skill in the art, cells of compositions and methods for production of HDAd 35 donor vectors can be 293 T cells (HEK293).

A helper may be engineered from wild-type or similarly propagation-competent vectors, such as a wild-type or propagation-competent Ad5 vector or Ad35 vector. As those of skill in the art will appreciate, one strategy that can be used in engineering of a helper vector is deletion or other functional disruption of E1 gene expression. The E1 region, located in the 5′ portion of adenoviral genomes, encodes proteins required for wild-type expression of the early and late genes. E1 deletion reduces or eliminates expression of certain viral genes controlled by E1, and E1-deleted helper viruses are replication-defective. Accordingly, E1-deficient helper virus can be propagated using cell lines that express E1. For example, where an E1-deficient Ad35 helper vector is engineered to encode an Ad5 E4orf6, the helper vector can be propagated in a cell line that expresses Ad5 E1, and where an E1-deficient Ad35 helper vector encodes an Ad5 E4orf6, the helper vector can be propagated in a cell line that expresses Ad5 E1. In one exemplary cell type for HDAd35 vector production, HEK293 cells express Ad5 E1 b55k, which is known to form a complex with Ad5 E4 protein ORF6. Table 2 provides an example summary of expression products encoded by an Ad35 genome (see Gao, Gene Ther. 10:1941-1949, 2003).

TABLE 2
Predicated translational features of the Ad35 genome.

| | | |
|---|---|---|
| Features | From | To |

E1 and pIX regions

| | | | |
|---|---|---|---|
| E1A 261R | | 569 | 1148 |
| | Join | 1233 | 1441 |
| E1A 230R | | 569 | 1055 |
| | Join | 1233 | 1441 |
| E1A 58R | | 569 | 640 |
| | Join | 1233 | 1337 |
| E1B 214R (small T antigen) | | 1611 | 2153 |
| E1B 494R (large T antigen) | | 1916 | 3400 |
| pIX | | 3484 | 3903 |
| ORF-1 | | 2366 | 2689 |

E2 and IVa2 regions (complementary strand)

| | | | |
|---|---|---|---|
| IVa2 | | 5579 | 5590 |
| | Join | 3966 | 5300 |
| E2B DNA pol | | 5069 | 8437 |
| E2B pTP | | 8440 | 10356 |
| E2A DBF | | 22414 | 23415 |
| ORF-2 | | 5988 | 6482 |
| ORF-3 | | 7847 | 8257 |
| ORF-4 | | 15663 | 15971 |
| ORF-5 | | 15743 | 16216 |
| ORF-6 | | 16457 | 17041 |
| ORF-7 | | 17543 | 17938 |
| ORR-8 | | 17994 | 18713 |
| ORF-9 | | 21858 | 22436 |
| ORF-10 | | 22128 | 22502 |
| ORF-11 | | 23027 | 23488 |

E3 region

| | | |
|---|---|---|
| E3 12.2K protein | 27198 | 27515 |
| E3 15.0K protein | 27469 | 27864 |
| E3 18.5K protein | 27849 | 28349 |
| E3 20.3K protein | 28369 | 28914 |
| E3 20.6K protein | 28932 | 29495 |
| E3 15.2K protein | 29817 | 30221 |
| E3 15.3K protein | 30214 | 30621 |
| ORF-12 | 25693 | 26019 |
| ORF-13 | 27908 | 28240 |

E4 region (complementary strand)

| | | |
|---|---|---|
| E4 299R | 32075 | 32974 |
| E4 145R | 33604 | 34041 |
| E4 125R | 34038 | 34415 |
| E4 117R | 33254 | 33607 |
| E4 122R | 32877 | 33245 |
| ORF-14 | 33100 | 33609 |
| VA RNA region | 10433 | 10594 |

L region

| | | | |
|---|---|---|---|
| L1 52, 55K | | 10653 | 11819 |
| L1 IIIa | | 11845 | 13608 |
| L2 III (penton base) | | 13690 | 15375 |
| L2 pVII | | 15383 | 15961 |
| L2 V | | 16004 | 17059 |
| L3 pVI | | 17399 | 18139 |
| L3 II (hexon) | | 18255 | 21113 |
| L3 23K (protease) | | 21150 | 21779 |
| L4 100K | | 23446 | 25884 |
| L4 22K | | 25616 | 26191 |
| L4 33K | | 25616 | 25934 |
| | Join | 26104 | 26465 |
| L4 pVIII | | 26515 | 27198 |
| L5 IV(fiber) | | 30826 | 31797 |

The present disclosure includes, among other things, HDAd35 donor vectors and genomes that include Ad35 ITRs (e.g., a 5′ Ad35 ITR and a 3′ ITR), e.g., where two Ad35 ITRs flank a payload. The present disclosure includes, among other things, HDAd35 donor vectors and genomes that include an Ad35 packaging sequence or a functional fragment thereof. The present disclosure includes, among other things, HDAd35 donor vectors and genomes in which E1 or a fragment thereof is deleted (e.g., where the E1 deletion includes deletion of nucleotides 481-3112 of GenBank Accession No. AX049983 or corresponding positions of another Ad35 vector sequence provided herein). The present disclosure includes, among other things, HDAd35 vectors and genomes in which E3 or a fragment thereof is deleted (e.g., where the E3 deletion includes deletion of nucleotides 27609 to 30402 or 27435-30542 of GenBank Accession No. AX049983 or corresponding positions of another Ad35 vector sequence provided herein).

The present disclosure includes, among other things, Ad35 helper vectors and genomes that include two recombination site elements that flank a packaging sequence or functionally contributing (e.g., functionally required) fragment thereof, each recombination site element including a recombination site, where the two recombination sites are sites for the same recombinase. Construction of an Ad35 helper vector, as noted above, cannot be predictably engineered from existing knowledge relating to other vectors. To the contrary, relevant sequences of Ad35 are very different from, e.g., corresponding sequences of Ad5 (compare, e.g., the 5′ 600 to 620 nucleotides of Ad35 and Ad5). Moreover, packaging sequence are serotype-specific. The Ad35 packaging sequence includes sequences that correspond to at least Ad5 packaging single sequences AI, AII, AIII, AIV, and AV. Accordingly, production of an Ad35 helper vector requires several unpredictable determinations, including (1) identification of the Ad35 packaging sequence or functionally contributing (e.g., functionally required) fragment thereof to be flanked by recombinase sites (e.g., loxP sites) by insertion of recombinase site elements into the subject genome, which is not straightforward where sequence similarity is limited; (2) identification of recombinase site element insertions that do not negate propagation of the helper vector (under conditions where the packaging sequence or functionally contributing (e.g., functionally required) fragment thereof is not excised), which cannot be predicted; and/or (3) identification of spacing between the recombination site elements that permits efficient deletion of the packaging sequence or functionally contributing (e.g., functionally required) fragment thereof while reducing helper virus packaging during production of HDAd35 donor vectors (e.g., in a cre recombinase-expressing cell line such as the 116 cell line).

The present disclosure includes a plurality of exemplary Ad35 helper vectors and genomes that (1) include loxP sites flanking a functionally contributing or functionally required fragment of the Ad35 packaging sequence, at least in that recombination of the loxP sites causing excision of the flanked sequence reduces propagation of the vector by, e.g., at least 20%, 30%, 40%, 50%, 60%, 70%, 80%, 90%, 91%, 92%, 93%, 94%, 95%, 96%, 97%, 98%, 99%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, or 100%), optionally where percent propagation is measured as the number of viral particles produced by propagation of excised vector (recombinase site-flanked sequence excised) as compared to complete vector (recombinase site-flanked sequence not excised) or wild-type Ad35 vector under comparable conditions.

In at least one exemplary Ad35 helper vector, a recombinase site element (e.g., a loxP element) is inserted after nucleotide 178 and a recombinase site element (e.g., a loxP element) is inserted after nucleotide 437. Excision of the loxP-flanked sequence removes packaging sequence sequences AI to AIV. In certain such embodiments, deletion of nucleotides 345-3113 removes the E1 gene as well as packaging single sequences AVI and AVII. Accordingly, the flanked packaging sequence or fragment thereof corresponds to positions 179-344. Vectors according to this description were shown to propagate.

In at least one exemplary Ad35 helper vector, a recombinase site element (e.g., a loxP element) is inserted after nucleotide 178 and a recombinase site element (e.g., a loxP element) is inserted after nucleotide 481, where nucleotides 179-365 are deleted (removing packaging sequence sequences AI to AV, such that remaining sequences AVI and AVII are in the nucleic acid sequence flanked by the recombinase site elements. In certain such embodiments, deletion of nucleotides 482-3113 removes the E1 gene. Accordingly, the flanked packaging sequence or fragment thereof corresponds to positions 366-481. Vectors according to this description were shown to propagate.

In at least one exemplary Ad35 helper vector, a recombinase site element (e.g., a loxP element) is inserted after nucleotide 154 and a recombinase site element (e.g., a loxP element) is inserted after nucleotide 481, In certain such embodiments, deletion of nucleotides 482-3113 removes the E1 gene. Accordingly, the flanked packaging sequence or fragment thereof corresponds to positions 155-481. Vectors according to this description were shown to propagate.

In at least one exemplary Ad35 helper vector, a recombinase site element (e.g., a loxP element) is inserted after nucleotide 158 and a recombinase site element (e.g., a loxP element) is inserted after nucleotide 480. Vectors according to this description were shown to propagate. In certain such embodiments, nucleotides 27388-30402 including E3 region are deleted. In certain embodiments, the vector is an Ad35++ vector.

In at least one exemplary Ad35 helper vector, a recombinase site element (e.g., a loxP element) is inserted after nucleotide 158 and a recombinase site element (e.g., a loxP element) is inserted after nucleotide 446. Vectors according to this description were shown to propagate. In certain such embodiments, nucleotides 27388-30402 including E3 region are deleted. In certain embodiments, the vector is an Ad35++ vector.

In at least one exemplary Ad35 helper vector, a recombinase site element (e.g., a loxP element) is inserted after nucleotide 179 and a recombinase site element (e.g., a loxP element) is inserted after nucleotide 480. Vectors according to this description were shown to propagate. In certain such embodiments, nucleotides 27388-30402 including E3 region are deleted. In certain embodiments, the vector is an Ad35++ vector.

In at least one exemplary Ad35 helper vector, a recombinase site element (e.g., a loxP element) is inserted after nucleotide 206 and a recombinase site element (e.g., a loxP element) is inserted after nucleotide 480. Vectors according to this description were shown to propagate. In certain such embodiments, nucleotides 27,388-30,402 including E3 region are deleted. In certain embodiments, nucleotides 27,607-30,409 or 27,609-30,402 are deleted. In certain embodiments, nucleotides 27,240-27,608 are not deleted.

In at least one exemplary Ad35 helper vector, a recombinase site element (e.g., a loxP element) is inserted after nucleotide 139 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 158 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 179 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 201 and a recombinase site element (e.g., a loxP element) is inserted after nucleotide 446. In certain such embodiments, nucleotides 27609-30402 are deleted.

In at least one exemplary Ad35 helper vector, a recombinase site element (e.g., a loxP element) is inserted after nucleotide 158 and a recombinase site element (e.g., a loxP element) is inserted after nucleotide 481. In certain such embodiments, nucleotides 27609-30402 are deleted.

In at least one exemplary Ad35 helper vector, a recombinase site element (e.g., a loxP element) is inserted after nucleotide 179 and a recombinase site element (e.g., a loxP element) is inserted after nucleotide 384. In certain such embodiments, nucleotides 27609-30402 are deleted.

In at least one exemplary Ad35 helper vector, a recombinase site element (e.g., a loxP element) is inserted after nucleotide 179 and a recombinase site element (e.g., a loxP element) is inserted after nucleotide 481. In certain such embodiments, nucleotides 27609-30402 are deleted.

In at least one exemplary Ad35 helper vector, a recombinase site element (e.g., a loxP element) is inserted after nucleotide 206 and a recombinase site element (e.g., a loxP element) is inserted after nucleotide 481. In certain such embodiments, nucleotides 27609-30402 are deleted.

An additional optional engineering consideration can be engineering of a helper genome having a size that permits separation of helper vector from HDAd35 donor vector by centrifugation, e.g., by CsCl ultracentrifugation. One means of achieving this result is to increase the size of the helper genome as compared to a typical Ad35 genome, which has a wild-type length of 34,794 bp. In particular, adenoviral genomes can be increased by engineering to at least 104% of wild-type length. Certain helper vectors of the present disclosure include the Ad35 E1 region and E4 region, delete the E3 region, and can accommodate a payload and/or stuffer sequence.

Ad35 helper vectors can be used for production of Ad35 donor vectors. Production of HDAd35++ vectors can include co-transfection of a plasmid containing the HDAd vector genome and a packaging-defective helper virus that provides structural and non-structural viral proteins. The helper virus genome can rescue propagation of the Ad35 donor vector and Ad35 donor vector can be produced, e.g., at a large scale, and isolated. Various protocols are known in the art, e.g., at Palmer et al., 2009 Gene Therapy Protocols. Methods in Molecular Biology, Volume 433. Humana Press; Totowa, N.J.: 2009. pp. 33-53.

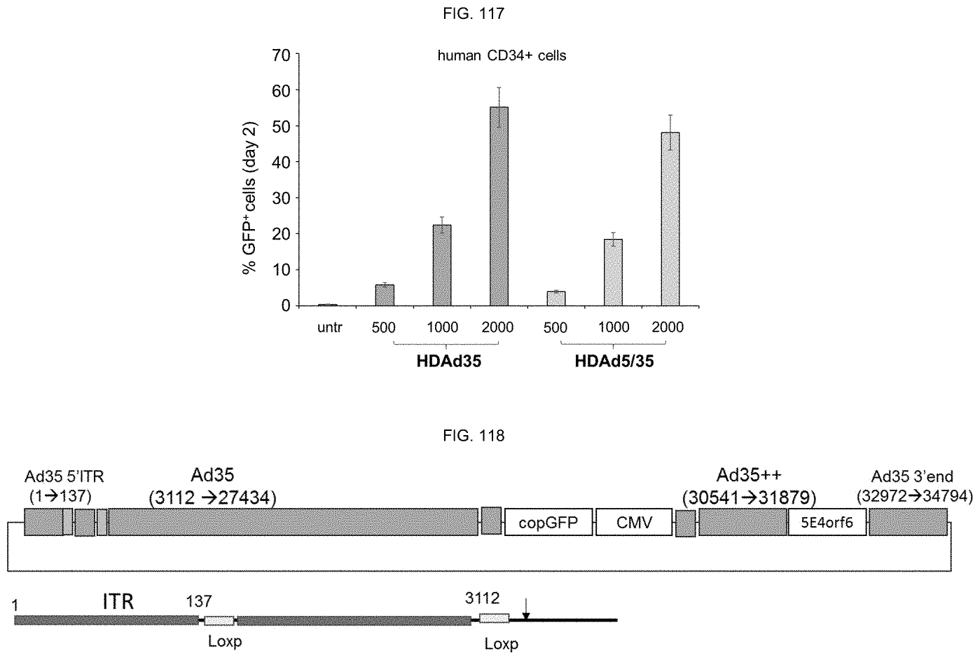

The present disclosure includes exemplary data demonstrating that HDAd35 donor vectors of the present disclosure perform comparably to HDAd5/35 donor vectors in transduction of human CD34+ cells, as measured by percent of contacted cells expressing a payload coding sequence encoding GFP. Results were confirmed at multiple MOIs ranging from 500 to 2000 vector particles per contacted cell. Exemplary experiments were conducted using HDAd35 donor vectors used in generating exemplary data were produced using an Ad35 helper vector as disclosed above, where loxP sites flanked nucleotides 366-481 (see, e.g., FIG. 117).

Various exemplary donor vectors are provided herein. The present disclosure provides, as non-limiting examples, HDAd35 donor genomes as set forth in Tables 3-6.

TABLE 3
Exemplary HDAd35 donor vector according to SEQ ID NO: 304.

| | Position in |
|---|---|
| Sequence Feature | SEQ ID NO: 304 |
| Ad35 5′ (including ITR, Packaging Sequence) | Start: 1 End: 481 |
| FRT recombinase direct repeat | Start: 14126 End: 14159 |
| | (Complementary) |
| pT4 transposase inverted repeat | Start: 14220 End: 14463 |
| EF1α promoter | Start: 14491 End: 15825 |
| mgmt^{P140K} selection cassette | Start: 15843 End: 16466 |
| polyA sequence | Start: 16484 End: 16705 |
| pT4 transposase inverted repeat | Start: 16735 End: 17000 |
| FRT recombinase direct repeat | Start: 17107 End: 17140 |
| | (Complementary) |
| Ad35 3′ (including ITR) | Start: 28823 End: 29230 |

TABLE 4
Exemplary HDAd35 donor vector according to SEQ ID NO: 305

| | Position in SEQ |
|---|---|
| Sequence Feature | ID NO: 305 |
| Ad35 5′ (including ITR, Packaging Sequence) | Start: 1 End: 481 |
| FRT recombinase direct repeat | Start: 14126 End: 14159 |
| | (Complementary) |
| pT4 transposase inverted repeat | Start: 14220 End: 14463 |
| EF1α promoter | Start: 14478 End: 15812 |
| mgmt^{P140K} selection cassette | Start: 15830 End: 16450 |
| 2A peptide-encoding sequence | Start: 16451 End: 16522 |
| GFP-encoding sequence | Start: 16523 End: 17242 |
| SV40 polyA sequence | Start: 17269 End: 17390 |
| pT4 transposase inverted repeat | Start: 17501 End: 17766 |
| FRT recombinase direct repeat | Start: 17873 End: 17906 |
| | (Complementary) |
| Ad35 3′ (including ITR) | Start: 29589 End: 29996 |

TABLE 5
Exemplary HDAd35 donor vector according to SEQ ID NO: 288.

| | Position in |
|---|---|
| Sequence Feature | SEQ ID NO: 288 |
| Ad35 5′ (including ITR, Packaging Sequence) | Start: 1 End: 481 |
| FRT recombinase direct repeat | Start: 14126 End: 14159 |
| | (Complementary) |
| pT4 transposase inverted repeat | Start: 14220 End: 14463 |
| EF1α promoter | Start: 14478 End: 15812 |
| mgmt^{P140K} selection cassette | Start: 15830 End: 16450 |
| 2A peptide-encoding sequence | Start: 16451 End: 16522 |
| mCherry-encoding sequence | Start: 16526 End: 17230 |
| SV40 polyA sequence | Start: 17259 End: 17380 |
| pT4 transposase inverted repeat | Start: 17491 End: 17756 |
| FRT recombinase direct repeat | Start: 17863 End: 17896 |
| | (Complementary) |
| Ad35 3′ (including ITR) | Start: 29579 End: 29986 |

TABLE 6
Exemplary support vector according to SEQ ID NO: 289.

| | Position in |
|---|---|
| Sequence Feature | SEQ ID NO: 289 |
| Ad35 5′ (including ITR, Packaging Sequence) | Start: 1 End: 481 |
| PGK promoter | Start: 14103 End: 14614 |
| SB100x transposase-encoding sequence | Start: 14763 End: 15785 |
| BGH polyA sequence | Start: 15811 End: 16128 |
| B-globin polyA sequence | Start: 16088 End: 16376 |
| | (Complementary) |
| Flpe recombinase-encoding sequence | Start: 16488 End: 17759 |
| | (Complementary) |
| EF1α promoter | Start: 17780 End: 18895 |
| | (Complementary) |
| Ad35 3′ (including ITR) | Start: 29751 End: 30158 |

TABLE 7
Exemplary Ad35 helper vector according to SEQ ID NO: 286

| | Position in |
|---|---|
| Sequence Feature | SEQ ID NO: 286 |
| Ad35 5′ (including ITR)(Ad35 nt 1-178) | Start: 2582 End: 2759 |
| LoxP recombinase site | Start: 2768 End: 2801 |
| Ad35 packaging sequence (Ad35 nt 179-344) | Start: 2808 End: 2973 |
| LoxP recombinase site | Start: 2974 End: 3007 |
| Ad35 sequence (Ad35 nt 3112-27435) | Start: 3016 End: 27338 |
| Lambda-1 sequence | Start: 27393 End: 29862 |
| | (Complementary) |
| BGH polyA sequence | Start: 30176 End: 30390 |
| CopGFP-encoding sequence | Start: 30415 End: 31080 |
| | (Complementary) |
| CMV promoter | Start: 31127 End: 31779 |
| | (Complementary) |
| Lambda-2 sequence | Start: 31831 End: 33360 |
| Ad35 sequence (Ad35 nt 30544-31879) | Start: 33421 End: 34756 |
| Ad5 E4orf6 sequence | Start: 34752 End: 35866 |
| Ad35 3′ (including ITR) | Start: 35864 End: 37686 |
| (Ad35 nt 32972-34794) | |

TABLE 8
Exemplary Ad35 helper vector according to SEQ ID NO: 51.

| | Position in |
|---|---|
| Sequence Feature | SEQ ID NO: 51 |
| Ad35 5′ (including ITR) (Ad35 nt 1-178) | Start: 2582 End: 2759 |
| LoxP recombinase site | Start: 2768 End: 2801 |
| Ad35 packaging sequence (Ad35 nt 366-481) | Start: 2808 End: 2923 |
| LoxP recombinase site | Start: 2924 End: 2957 |
| Ad35 sequence (Ad35 nt 3112-2743) | Start: 2966 End: 27288 |
| Lambda-1 sequence | Start: 27343 End: 29812 |
| | (Complementary) |
| BGH polyA sequence | Start: 30126 End: 30340 |
| CopGFP-encoding sequence | Start: 30365 End: 31030 |
| | (Complementary) |
| CMV promoter | Start: 31077 End: 31729 |
| | (Complementary) |
| Lambda-2 sequence | Start: 31781 End: 33310 |
| Ad35 sequence (Ad35 nt 30544-31879) | Start: 33371 End: 34706 |
| Ad5 E4orf6 sequence | Start: 34702 End: 35816 |
| Ad35 3′ (including ITR) | Start: 35814 End: 37636 |
| (Ad35 nt 32972-34794) | |

TABLE 9
Exemplary Ad35 helper vector according to SEQ ID NO: 52.

| | Position in |
|---|---|
| Sequence Feature | SEQ ID NO: 52 |
| Ad35 5′ (including ITR) (Ad35 nt 1-154) | Start: 2582 End: 2735 |
| LoxP recombinase site | Start: 2744 End: 2777 |
| Ad35 packaging sequence (Ad35 nt 155-481) | Start: 2784 End: 3110 |
| LoxP recombinase site | Start: 3111 End: 3144 |
| Ad35 sequence (Ad35 nt 3112-27435) | Start: 3153 End: 27475 |
| Lambda-1 sequence | Start: 27530 End: 29999 |
| | (Complementary) |
| BGH polyA sequence | Start: 30313 End: 30527 |
| CopGFP-encoding sequence | Start: 30552 End: 31217 |
| | (Complementary) |
| CMV promoter | Start: 31264 End: 31916 |
| | (Complementary) |
| Lambda-2 sequence | Start: 31968 End: 33497 |
| Ad35 sequence (Ad35 nt 30544-31879) | Start: 33558 End: 34893 |
| Ad5 E4orf6 sequence | Start: 34889 End: 36003 |
| Ad35 3′ (including ITR) | Start: 36001 End: 37823 |
| (Ad35 nt 32972-34794) | |

### I(C). Gene Therapy Vector Payloads

Ad35 and Ad5/35 donor vectors and genomes of the present disclosure can include a variety of nucleic acid payloads that can include any of one or more coding sequences that encode one or more expression products, one or more regulatory sequences operably linked to a coding sequence, one or more stuffer sequences, and the like. In various embodiments, the payload is engineered in order to achieve a desired result such as a therapeutic effect in a host cell or system, e.g., expression of a protein of therapeutic interest or of expression of a gene editing system, e.g., a CRISPR/Cas system or base editing system, to generate a sequence modification of therapeutic interest. In some embodiments, a payload can include a gene. A gene can include not only coding sequences but also regulatory regions such as promoters, enhancers, termination regions, locus control regions (LCRs), termination and polyadenylation signal elements, splicing signal elements, and the like. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

A payload can include a single gene or multiple genes. A payload can include a single regulatory sequence or a plurality of regulatory sequences. A payload can include a single coding sequence or a plurality of coding sequences. A payload can include a plurality of coding sequences where the individual expression products of the coding sequences function together, e.g., as in the case of an endonuclease and a guide RNA, or independently, e.g., as two separate proteins that do not directly or indirectly bind. In some instances, a plurality of coding sequences can function cooperatively, e.g., where an endonuclease and guide RNA cause an increase expression of coding sequence endogenous to a host cell or system and the payload further encoded and expresses a protein having at least one biological activity corresponding to that of a protein encoded by the endogenous coding sequence. As will be appreciated by those of skill in the art, any payload-encoded expression products provided herein that are not encoded by the canonical wild-type Ad35 genome can be referred to herein as a heterologous expression product.

### I(C)(i). Payload Expression Products

A payload of an adenoviral donor vector or adenoviral donor genome of the present disclosure can include one or more coding sequences that encode any of a variety of expression products. Exemplary expression products include proteins, including without limitation replacement therapy proteins for treatment of diseases or conditions characterized by low expression or activity of a biologically active protein as compared to a reference level. Exemplary expression products include CRISPR/Cas and base editor systems. Exemplary expression products include antibodies, CARs, and TCRs. Exemplary expression products include small RNAs. In various embodiments, integration of all or a portion of a donor vector payload into a host cell genome is not required in order for delivery to the target cell of a donor vector or genome to produce an intended or target effect, e.g., in certain instances in which the intended or target effect includes editing of the host cell genome by a CRISPR system or base editor system. In various embodiments, integration of all or a portion of a donor vector payload is required or preferred in order for delivery to the target cell of a donor vector or genome to produce an intended or target effect, e.g., where expression of a payload-encoded expression product is desired in progeny cells of a transduced target cell. In various embodiments, a payload can include a nucleic acid sequence engineered for integration into a host cell genome (an “integration element”), e.g., by recombination or transposition.

A gene sequence encoding one or more therapeutic proteins can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

Particular examples of therapeutic genes and/or gene products include γ-globin, Factor VIII, 1C, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, and SLC46A1; FANC family genes including FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3); soluble CD40; CTLA; Fas L; antibodies to CD4, CD5, CD7, CD52, etc.; antibodies to IL1, IL2, IL6; an antibody to TCR specifically present on autoreactive T cells; IL4; IL10; IL12; 1L13; IL1Ra, sIL1RI, sIL1R11; sTNFRI; sTNFRII; antibodies to TNF; P53, PTPN22, and DRB1*1501/DQB1*0602; globin family genes; WAS; phox; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; C9ORF72 and other therapeutic genes described herein.

A therapeutic gene can be selected to provide a therapeutically effective response against diseases related to red blood cells and clotting. In particular embodiments, the disease is a hemoglobinopathy like thalassemia, or a sickle cell disease/trait. The therapeutic gene may be, for example, a gene that induces or increases production of hemoglobin; induces or increases production of β-globin, γ-globin, or α-globin; or increases the availability of oxygen to cells in the body. The therapeutic gene may be, for example, HBB or CYB5R3. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments may, for example, increase or induce the production of coagulation/clotting factors VIII and IX; improve the functioning of coagulation/clotting factors VIII and IX, or reduce clotting time in subjects.

In various embodiments of the present disclosure, a donor vector encodes a globin gene, wherein the globin protein encoded by the globin gene is selected from a γ-globin, a β-globin, and/or an α-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or α-globin is a component of fetal and/or adult hemoglobin and is therefore useful in various vectors disclosed herein.

In various embodiments, increasing expression of a globin protein can refer to any of one or more of (i) increasing the amount, concentration, or expression (e.g., transcription or translation of nucleic acids encoding) in a cell or system of globin protein having a particular sequence; (ii) increasing the amount, concentration, or expression (e.g., transcription or translation of nucleic acids encoding) in a cell or system of globin protein of a particular type (e.g., the total amount of all proteins that would be identified as γ-globin (or alternatively β-globin or α-globin) by those of skill in the art or as set forth in the present specification) without respect to the sequences of the proteins relative to each other; and/or (iii) expressing in a cell or system a heterologous globin protein, e.g., a globin protein not encoded by a host cell prior to gene therapy.

The following references describe particular exemplary sequences of functional globin genes. References 1˜4 relate to α-type globin sequences and references 4-12 relate to β-type globin sequences (including β and γ globin sequences), which sequences are hereby incorporated by reference: (1) GenBank Accession No. Z84721 (Mar. 19, 1997); (2) GenBank Accession No. NM_000517 (Oct. 31, 2000); (3) Hardison et al., J. Mol. Biol. (1991) 222(2):233-249; (4) A Syllabus of Human Hemoglobin Variants (1996), by Titus et al., published by The Sickle Cell Anemia Foundation in Augusta, Ga. (available online at globin.cse.psu.edu); (5) GenBank Accession No. J00179 (Aug. 26, 1993); (6) Tagle et al., Genomics (1992) 13(3):741-760; (7) Grovsfeld et al., Cell (1987) 51(6):975-985; (8) Li et al., Blood (1999) 93(7):2208-2216; (9) Gorman et al., J. Biol. Chem. (2000) 275(46):35914-35919; (10) Slightom et al., Cell (1980) 21(3):627-638; (11) Fritsch et al., Cell (1980) 19(4): 959-972; (12) Marotta et al., J. Biol. Chem. (1977) 252(14):5040-5053. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS.; 92:6728-6732, 1995.

An exemplary amino acid sequence of hemoglobin subunit β is provided, for example, at NCBI Accession No. P68871. An exemplary amino acid sequence for β-globin is provided, for example, at NCBI Accession No. NP_000509.

In addition to therapeutic genes and/or gene products, the transgene can also encode for therapeutic molecules, such as checkpoint inhibitor reagents, chimeric antigen receptor molecules specific to one or more cancer antigens, and/or T-cell receptors specific to one or more cancer antigens.

As another example, a therapeutic gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay Sachs; Pompe disease; Gaucher disease; Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. Macrosephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a therapeutic gene can be selected to provide a therapeutically effective response against a hyperproliferative disease. In particular embodiments, the hyperproliferative disease is cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include (in addition to those listed elsewhere herein) 101F6, 123F2 (RASSFI), 53BP2, abl, ABLI, ADP, aFGF, APC, ApoAl, ApoAlV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, EIA, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p57, p73, p300, PGS, PIM1, PL6, PML, PTEN, raf, Rap1A, ras, Rb, RBI, RET, rks-3, ScFv, scFV ras, SEM A3, SRC, TALI, TCL3, TFPI, thrombospondin, thymidine kinase, TNF, TP53, trk, T-VEC, VEGF, VHL, VVT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

As another example, a therapeutic gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include a2p1; avp3; avp5; avp63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/a2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

In various embodiments, a vector or genome of the present disclosure, e.g., an Ad35 helper vector or Ad35 helper genome, encodes and/or expresses an Anti-CRISPR (Acr) protein, e.g., derived from phage, that inhibits normal activity of CRISPR/Cas.

### I(C)(i)(a). Binding Domain, Antibody, CAR, and TCR Payload Expression Products

The present disclosure includes a variety of binding domains. Antibodies are one example of binding domains and include whole antibodies or binding fragments of an antibody, e.g., Fv, Fab, Fab′, F(ab′)2, and single chain (sc) forms and fragments thereof (e.g., scFvs) that bind specifically to a cellular marker. Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies. Functional fragments thereof, include a single-domain antibody such as a heavy chain variable domain (VH), a light chain variable domain (VL) and a variable domain (VHH) of camelid derived nanobody, and the like.

In some instances, scFvs can be prepared according to methods known in the art (see, for example, Bird et al., Science 242:423-426, 1988; and Huston et al., Proc. Natl. Acad. Sci. USA 85:5879-5883, 1988). ScFv molecules can be produced by linking VL and VH regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, WO2006/020258, and WO2007/024715.

An scFv can include a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. In particular embodiments, the linker sequence may include any naturally occurring amino acid. Generally, linker sequences that are used to connect the VL and VH of an scFv are five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies.

In some embodiments, the linker sequence of an scFv includes the amino acids glycine and serine. In particular embodiments, the linker sequence includes sets of glycine and serine repeats such as from one to ten repeats of (GlyxSery)n, wherein x and y are independently an integer from 0 to 10 provided that x and y are not both 0 and wherein n is an integer of 1, 2, 3, 4, 5, 6, 7, 8, 9 or 10) and wherein linked VH-VL regions form a functional immunoglobulin-like binding domain (e.g., scFv, scTCR). Particular examples include (Gly4Ser)n, (Gly3Ser)n(Gly4Ser)n, (Gly3Ser)n(Gly2Ser)n, (Gly3Ser)n(Gly4Ser)1, (Gly4Ser)1, (Gly3Ser)1, or (Gly2Ser)1. In particular embodiments, the linker is (Gly4Ser)4 or (Gly4Ser)3. As indicated through reference to scTCR above, such linkers can also be used to link T cell receptor Vα/β and Cα/β chains (e.g., Vα-Cα, Vβ-Cβ, Vα-Vβ).

Additional examples include scFv-based grababodies and soluble VH domain antibodies.

These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. In one aspect, the antigen binding domain is humanized. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., PNAS, 91:969-973, 1994), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25, 2002, Caldas et al., Protein Eng., 13(5):353-60, 2000, Morea et al., Methods, 20(3):267-79, 2000, Baca et al., J. Biol. Chem., 272(16): 10678-84, 1997, Roguska et al., Protein Eng., 9(10):895-904, 1996, Couto et al., Cancer Res., 55 (23 Supp):5973s-5977s, 1995, Couto et al., Cancer Res., 55(8):1717-22, 1995, Sandhu, Gene, 150(2):409-10, 1994, and Pedersen et al., J. Mol. Biol., 235(3):959-73, 1994. Often, framework residues in the framework regions will be substituted with the corresponding residue from the CDR donor antibody to alter, for example improve, cellular marker binding. These framework substitutions are identified by methods well-known in the art, e.g., by modeling of the interactions of the CDR and framework residues to identify framework residues important for cellular marker binding and sequence comparison to identify unusual framework residues at particular positions. (See, e.g., U.S. Pat. No. 5,585,089; and Riechmann et al., Nature, 332:323, 1988).

Antibodies and other binding domains that specifically bind a particular cellular marker can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, U.S. Pat. Nos. 6,291,161 and 6,291,158). Phage display libraries of partially or fully synthetic antibodies are available and can be screened for an antibody or fragment thereof that can bind to a cellular marker. For example, binding domains may be identified by screening a Fab phage library for Fab fragments that specifically bind to a cellular marker of interest (see Hoet et al., Nat. Biotechnol. 23:344, 2005). Phage display libraries of human antibodies are also available. Additionally, traditional strategies for hybridoma development using a cellular marker of interest as an immunogen in convenient systems (e.g., mice, HuMAb Mouse® (GenPharm Intl. Inc., Mountain View, Calif.), TC Mouse® (Kirin Pharma Co. Ltd., Tokyo, JP), KM-Mouse® (Medarex, Inc., Princeton, N.J.), llamas, chicken, rats, hamsters, rabbits, etc.) can be used to develop binding domains. In particular embodiments, antibodies specifically bind to a cellular marker preferentially expressed by a particular cancer cell type and do not cross react with nonspecific components or unrelated targets. Once identified, the amino acid sequence of the antibody and gene sequence encoding the antibody can be isolated and/or determined.

In particular embodiments, a therapeutic gene can encode an antibody or a binding fragment of an antibody, such as a Fab or an scFv. Exemplary antibodies (including scFvs) that can be expressed include those provided described in WO2014/164553A1, US2017/0283504, U.S. Pat. Nos. 7,083,785, 10,189,906, 10,174,095, WO2005102387, US2011/0206701A1, WO2014/179759A1, US2018/0037651A1, US2018/0118822A1, WO2008/047242A2, WO1996/016990A1, WO200/5103083A2, and WO1999/062526A2. Antibodies described above in relation to binding domains can also be used, as well as atezolizumab, blinatumomab, brentuximab, cetuximab, cirmtuzumab, farletuzumab, gemtuzumab, OKT3, oregovomab, promiximab, pembrolizumab, and trastuzumab.

Immune checkpoint inhibitors can also be used. Immune checkpoint inhibitors refer to compounds that inhibit the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. In particular embodiments, immune checkpoint inhibitors enhance the proliferation, migration, persistence and/or cytoxicity activity of CD8+ T cells in a subject and in particular the tumor-infiltrating of CD8+ T cells of the subject. Another exemplary immune checkpoint inhibitor includes a checkpoint inhibitor as disclosed in Example 4. Accordingly, exemplary immune checkpoint inhibitors of the present disclosure include αPD-L1γ1 antibody (alternatively referred to as αPD-L1γ1). αPD-L1γ1 is further described in Engeland et al. Mol Ther 22(11):1949-1959, 2014, which is herein incorporated by reference in its entirety and in particular with respect to anti-PD-L1 antibodies, nucleic acids encoding the same, and uses thereof.

Examples of PD-1 and PD-L1 antibodies are described in U.S. Pat. Nos. 7,488,802; 7,943,743; 8,008,449; 8,168,757; 8,217,149, WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., J. Exp. Med. 207:2175-86, 2010 and Sakuishi et al., J. Exp. Med. 207:2187-94, 2010). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011/155607, WO2013/006490 and WO2010/117057.

Additional particular immune checkpoint inhibitors include atezolizumab, BMS-936559, ipilimumab, MEDI0680, MEDI4736, MSB0010718C, pembrolizumab, pidilizumab, and tremelimumab. See also WO 1998/42752; WO 2000/37504; WO 2001/014424; WO 2004/035607; US 2005/0201994; US 2002/0039581; US 2002/086014; U.S. Pat. Nos. 5,811,097; 5,855,887; 5,977,318; 6,051,227; 6,984,720; 6,682,736; 6,207,156; 6,682,736; 7,109,003; 7,132,281; EP1212422B1; Hurwitz et al., Proc. Natl. Acad. Sci. USA, 95(17):10067-10071 (1998); Camacho et al., J. Clin. Oncology, 22(145): Abstract No. 2505, 2004 (antibody CP-675206); and Mokyr et al., Cancer Res, 58:5301-5304, 1998.

The present disclosure further includes antibodies and other binding domains that bind CD4, CD5, CD7, CD52, etc.; antibodies; antibodies to IL1, IL2, IL6; an antibody to TCR specifically present on autoreactive T cells; IL4; IL10; 1L12; 1L13; IL1Ra; sIL1 RI; sIL1R11; antibodies to TNF; ABCA3; ABCD1; ADA; AK2; APP; arginase; arylsulfatase A; AIAT; CD3D; CD3E; CD3G; CD3Z; CFTR; CHD7; chimeric antigen receptor (CAR); CIITA; CLN3; complement factor, COROIA; CTLA; C1 inhibitor; C9ORF72; DCLREIB; DCLREIC; decoy receptors; DKC1; DRB1*1501/DQB1*0602; dystrophin; enzymes; Factor VIII, FANC family genes (FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)); Fas L; FUS; GATA1; globin family genes (ie. γ-globin); F8; glutaminase; HBA1; HBA2; HBB; IL7RA; JAK3; LCK; LIG4; LRRK2; NHEJ1; NLX2.1; neutralizing antibodies; ORAI1; PARK2; PARK7; phox; PINK1; PNP; PRKDC; PSEN1; PSEN2; PTPN22; PTPRC; P53; pyruvate kinase; RAG1; RAG2; RFXANK; RFXAP; RFX5; RMRP; ribosomal protein genes; SFTPB; SFTPC; SOD1; soluble CD40; STIM1; sTNFRI; sTNFRII; SLC46A1; SNCA; TDP43; TERT; TERC; TINF2; ubiquilin 2; WAS; WHN; ZAP70; yC; and other therapeutic genes described herein.

An alternative source of binding domains includes sequences that encode random peptide libraries or sequences that encode an engineered diversity of amino acids in loop regions of alternative non-antibody scaffolds, such as scTCR (see, e.g., Lake et al., Int. Immunol. 11:745, 1999; Maynard et al., J. Immunol. Methods 306:51, 2005; U.S. Pat. No. 8,361,794), fibrinogen domains (see, e.g., Weisel et al., Science 230:1388, 1985), Kunitz domains (see, e.g., U.S. Pat. No. 6,423,498), designed ankyrin repeat proteins (DARPins; Binz et al., J. Mol. Biol. 332:489, 2003 and Binz et al., Nat. Biotechnol. 22:575, 2004), fibronectin binding domains (adnectins or monobodies; Richards et al., J. Mol. Biol. 326:1475, 2003; Parker et al., Protein Eng. Des. Selec. 18:435, 2005 and Hackel et al., J. Mol. Biol. 381:1238-1252, 2008), cysteine-knot miniproteins (Vita et al., 1995, Proc. Nat'l. Acad. Sci. (USA) 92:6404-6408; Martin et al., 2002, Nat. Biotechnol. 21:71, 2002 and Huang et al., Structure 13:755, 2005), tetratricopeptide repeat domains (Main et al., Structure 11:497, 2003 and Cortajarena et al., ACS Chem. Biol. 3:161, 2008), leucine-rich repeat domains (Stumpp et al., J. Mol. Biol. 332:471, 2003), lipocalin domains (see, e.g., WO 2006/095164, Beste et al., Proc. Nat'l. Acad. Sci. (USA) 96:1898, 1999 and Schonfeld et al., Proc. Nan. Acad. Sci. (USA) 106:8198, 2009), V-like domains (see, e.g., US 2007/0065431), C-type lectin domains (Zelensky and Gready, FEBS J. 272:6179, 2005; Beavil et al., Proc. Nan. Acad. Sci. (USA) 89:753, 1992 and Sato et al., Proc. Nan. Acad. Sci. (USA) 100:7779, 2003), mAb2 or Fc-region with antigen binding domain (Fcab™ (F-Star Biotechnology, Cambridge UK; see, e.g., WO 2007/098934 and WO 2006/072620), armadillo repeat proteins (see, e.g., Madhurantakam et al., Protein Sci. 21: 1015, 2012; WO 2009/040338), affilin (Ebersbach et al., J. Mol. Biol. 372: 172, 2007), affibody, avimers, knottins, fynomers, atrimers, cytotoxic T-lymphocyte associated protein-4 (Weidle et al., Cancer Gen. Proteo. 10:155, 2013), or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011).

Peptide aptamers include a peptide loop (which is specific for a cellular marker) attached at both ends to a protein scaffold. This double structural constraint increases the binding affinity of peptide aptamers to levels comparable to antibodies. The variable loop length is typically 8 to 20 amino acids and the scaffold can be any protein that is stable, soluble, small, and non-toxic. Peptide aptamer selection can be made using different systems, such as the yeast two-hybrid system (e.g., Gal4 yeast-two-hybrid system), or the LexA interaction trap system.

In particular embodiments, a binding domain binds the cellular marker CD33. In particular embodiments, the binding domain that binds CD33 is derived from one of gemtuzumab, aclizumab, or HuM195. In particular embodiments a CD33 binding domain is a human or humanized binding domain including a variable light chain including a CDRL1 sequence including SEQ ID NO: 91, a CDRL2 sequence including SEQ ID NO: 92, and a CDRL3 sequence including SEQ ID NO: 93, and a variable heavy chain including a CDRH1 sequence including SEQ ID NO: 94, a CDRH2 sequence including SEQ ID NO: 95, and a CDRH3 sequence including SEQ ID NO: 96.

In particular embodiments, a CD33 binding domain is a human or humanized scFv including a variable light chain including a CDRL1 sequence including SEQ ID NO: 97, a CDRL2 sequence including SEQ ID NO: 98, and a CDRL3 sequence including SEQ ID NO: 99, and a variable heavy chain including a CDRH1 sequence including SEQ ID NO: 100, a CDRH2 sequence including SEQ ID NO: 101, and a CDRH3 sequence including SEQ ID NO: 102. For more information regarding binding domains that bind CD33, see U.S. Pat. No. 8,759,494.

In particular embodiments, a sequence that binds human CD33 includes a variable light chain region including sequence SEQ ID NO: 103, and a variable heavy chain region including sequence SEQ ID NO: 104. In particular embodiments, a sequence that binds human CD33 includes a variable light chain region including sequence SEQ ID NO: 103, and a variable heavy chain region including sequence SEQ ID NO: 106.

In particular embodiments, a binding domain binds full-length CD33 (CD33FL). In particular embodiments, the binding domain that binds CD33FL is derived from at least one of 5D12, 8F5, 1H7, lintuzumab, or gemtuzumab. In particular embodiments, a CD33FL binding domain is human or humanized, including a variable light chain including a CDRL1 sequence including SEQ ID NO: 107, a CDRL2 sequence including SEQ ID NO: 108, a CDRL3 sequence including SEQ ID NO: 109), a CDRH1 sequence including SEQ ID NO: 110, a CDRH2 sequence including SEQ ID NO: 111, and a CDRH3 sequence including SEQ ID NO: 112. For more information regarding binding domains that bind CD33FL, see PCT/US17/42264.

In particular embodiments, a binding domain that binds human CD33FL includes a variable light chain region including sequence SEQ ID NO: 113), and a variable heavy chain region including sequence SEQ ID NO: 114.

In particular embodiments, a binding domain binds the cellular marker CD33DeltaE2 (CD33ΔE2). In particular embodiments, the binding domain that binds CD33ΔE2 is derived from at least one of 12B12, 4H10, 11D5, 13E11, 11D11, or 1H7. In particular embodiments, an CD33ΔE2 binding domain is human or humanized and includes a variable light chain including a CDRL1 sequence including SEQ ID NO: 115, a CDRL2 sequence including SEQ ID NO: 116, a CDRL3 sequence including SEQ ID NO: 117, a CDRH1 sequence including SEQ ID NO: 118, a CDRH2 sequence including SEQ ID NO: 11), and a CDRH3 sequence including SEQ ID NO: 120. For more information regarding binding domains that bind CD33ΔE2, see PCT/US17/42264.

In particular embodiments, a sequence that binds human CD33ΔE2 includes a variable light chain region including sequence SEQ ID NO: 121, and a variable heavy chain region including sequence SEQ ID NO: 122.

In particular embodiments, a binding domain binds the cellular marker Her2. In particular embodiments, the binding domain that binds HER2 is derived from trastuzumab (Herceptin). In particular embodiments, the binding domain includes a variable light chain including a CDRL1 sequence including SEQ ID NO: 12), a CDRL2 sequence including SEQ ID NO: 124, and a CDRL3 sequence including SEQ ID NO: 125, and a variable heavy chain including a CDRH1 sequence including SEQ ID NO: 126, a CDRH2 sequence including SEQ ID NO: 127, and a CDRH3 sequence including SEQ ID NO: 128.

In particular embodiments, a binding domain binds the cellular marker PD-L1. In particular embodiments, the binding domain that binds PD-L1 is derived from at least one of pembrolizumab or FAZ053 (Novartis). In particular embodiments, the binding domain includes a variable light chain including a CDRL1 sequence including SEQ ID NO: 129, a CDRL2 sequence including SEQ ID NO: 130, and a CDRL3 sequence including SEQ ID NO: 131, and a variable heavy chain including a CDRH1 sequence including SEQ ID NO: 132, a CDRH2 sequence including SEQ ID NO: 133, and a CDRH3 sequence including SEQ ID NO: 134.

An exemplary binding domain for PD-L1 can include or be derived from Avelumab or Atezolizumab. In particular embodiments, the variable heavy chain of Avelumab includes SEQ ID NO: 135. In particular embodiments, the variable light chain of Avelumab includes SEQ ID NO: 136.

In particular embodiments, the CDR regions of Avelumab include: CDRH1 including SEQ ID NO: 137; CDRH2 including SEQ ID NO: 138; CDRH3 including SEQ ID NO: 139; CDRL1 including SEQ ID NO: 140; CDRL2 including SEQ ID NO: 141; and CDRL3 including SEQ ID NO: 142. In particular embodiments, the variable heavy chain of Atezolizumab includes SEQ ID NO: 143. In particular embodiments, the variable light chain of Atezolizumab includes SEQ ID NO: 144.

In particular embodiments, the CDR regions of Atezolizumab include: CDRH including SEQ ID NO: 145; CDRH2 including SEQ ID NO: 146; CDRH3 including SEQ ID NO: 147; CDRL1 including SEQ ID NO: 148; CDRL2 including SEQ ID NO: 149; and CDRL3 including SEQ ID NO: 150.

In particular embodiments, a binding domain binds the cellular marker PSMA. In particular embodiments, the binding domain includes a variable light chain including a CDRL1 sequence including SEQ ID NO: 151, a CDRL2 sequence including SEQ ID NO: 152, a CDRL3 sequence including SEQ ID NO: 153. In particular embodiments, the binding domain includes a variable heavy chain including a CDRH1 sequence including SEQ ID NO: 154, a CDRH2 sequence including SEQ ID NO: 155, and a CDRH3 sequence including SEQ ID NO: 156.

In particular embodiments, a binding domain binds the cellular marker MUC16. In particular embodiments, the binding domain is human or humanized and includes a variable light chain including a CDRL1 sequence including SEQ ID NO: 157, a CDRL2 sequence including GAS, a CDRL3 sequence including SEQ ID NO: 158. In particular embodiments, the binding domain is human or humanized and includes a variable heavy chain including a CDRH1 sequence including SEQ ID NO: 159, a CDRH2 sequence including SEQ ID NO: 160, and a CDRH3 sequence including SEQ ID NO: 161.

In particular embodiments, a binding domain binds the cellular marker FOLR. In particular embodiments, the binding domain that binds FOLR is derived from farletuzumab. In particular embodiments, the binding domain includes a variable light chain including a CDRL1 sequence including SEQ ID NO: 162, a CDRL2 sequence including SEQ ID NO: 163, and a CDRL3 sequence including SEQ ID NO: 164, and a variable heavy chain including a CDRH1 sequence including SEQ ID NO: 165, a CDRH2 sequence including SEQ ID NO: 166, and a CDRH3 sequence including SEQ ID NO: 167.

An exemplary binding domain for mesothelin can include or be derived from Amatuximab. In particular embodiments, the variable heavy chain of Amatuximab includes SEQ ID NO: 168. In particular embodiments, the variable light chain of Amatuximab includes SEQ ID NO: 169.

In particular embodiments, the CDR regions of Amatuximab include: A CDRH1 sequence including SEQ ID NO: 170; a CDRH2 sequence including SEQ ID NO: 171; a CDRH3 sequence including SEQ ID NO: 172; a CDRL1 sequence including SEQ ID NO: 173; a CDRL2 sequence including (SEQ ID NO: 174; and a CDRL3 sequence including SEQ ID NO: 175.

In particular embodiments, a binding domain is a sc T cell receptor (scTCR) including Vα/β and Cα/β chains (e.g., Vα-Cα, Vβ-Cβ, Vα-Vβ) or including a Vα-Cα, Vβ-Cβ, Vα-Vβ pair specific for a cellular marker of interest (e.g., peptide-MHC complex).

In particular embodiments, a binding domain includes a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR Vα, Vβ, Cα, or Cβ, wherein each CDR includes zero changes or at most one, two, or three changes, from a TCR or fragment or derivative thereof that specifically binds to the targeted cellular marker.

In particular embodiments, a binding domain includes Vα, Vβ, Cα, and/or Cβ regions derived from or based on a Vα, Vβ, Cα, and/or Cβ of a known or identified TCR (e.g., a high-affinity TCR) and includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the Vα, Vβ, Cα, and/or Cβ of a known or identified TCR. An insertion, deletion or substitution may be anywhere in a Vα, Vβ, Cα, and/or Cβ region, including at the amino- or carboxy-terminus or both ends of these regions, provided that each CDR includes zero changes or at most one, two, or three changes and provides a target binding domain containing a modified Vα, Vβ, Cα, or Cβ region can still specifically bind its target with an affinity and action similar to wild type.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from a monoclonal antibody or fragment or derivative thereof that specifically binds to a cellular marker of interest.

In particular embodiments, a VL region in a binding domain of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), ora combination of the above-noted changes, when compared with the VL of the known monoclonal antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of a known monoclonal antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); A1-Lazikani et al., J Mol Biol 273: 927-948, 1997 (Chothia numbering scheme); Maccallum et al., J Mol Biol 262: 732-745, 1996 (Contact numbering scheme); Martin et al., Proc. Natl. Acad. Sci., 86: 9268-9272, 1989 (AbM numbering scheme); Lefranc et al., Dev Comp Immunol 27(1): 55-77, 2003 (IMGT numbering scheme); and Honegger and Pluckthun, J Mol Biol 309(3): 657-670, 2001 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering.

Particular cellular markers associated with prostate cancer include PSMA, VVT1, ProstateStem Cell antigen (PSCA), and SV40 T. Particular cellular markers associated with breast cancer include HER2 and ERBB2. Particular cellular markers associated with ovarian cancer include L1-CAM, extracellular domain of MUC16 (MUC-CD), folate binding protein (folate receptor), Lewis Y, mesothelin, and WT-1. Particular cellular markers associated with pancreatic cancer include mesothelin, CEA and CD24. Particular cellular markers associated with multiple myeloma include BCMA, GPRCSD, CD38, and CS-1. Particular markers associated with leukemia and/or lymphoma include CLL-1, CD123, CD33, and PD-L1.

Also contemplated are binding domains specific for infectious disease agents, for instance by binding to an infectious agent antigen. These include for instance viral antigens or other viral markers, for instance which are expressed by virally-infected cells. Exemplary viruses include adenoviruses, arenaviruses, bunyaviruses, coronaviruses, flaviviruses, hantaviruses, hepadnaviruses, herpesviruses, papillomaviruses, paramyxoviruses, parvoviruses, picornaviruses, poxviruses, orthomyxoviruses, retroviruses, reoviruses, rhabdoviruses, rotaviruses, spongiform viruses or togaviruses. In additional embodiments, viral antigen markers include peptides expressed by CMV, cold viruses, Epstein-Barr, flu viruses, hepatitis A, B, and C viruses, herpes simplex, HIV, influenza, Japanese encephalitis, measles, polio, rabies, respiratory syncytial, rubella, smallpox, varicella zoster or West Nile virus.

As further particular examples, cytomegaloviral antigens include envelope glycoprotein B and CMV pp65; Epstein-Barr antigens include EBV EBNAI, EBV P18, and EBV P23; hepatitis antigens include the S, M, and L proteins of HBV, the pre-S antigen of HBV, HBCAG DELTA, HBV HBE, hepatitis C viral RNA, HCV NS3 and HCV NS4; herpes simplex viral antigens include immediate early proteins and glycoprotein D; HIV antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include hemagglutinin and neuraminidase; Japanese encephalitis viral antigens include proteins E, M-E, M-E-NS1, NS1, NS1-NS2A and 80% E; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpI and gpII.

Additional particular exemplary viral antigen sequences include: Nef (66-97) (SEQ ID NO: 176), Nef (116-145) (SEQ ID NO: 177), Gag p17 (17-35) (SEQ ID NO: 178), Gag p17-p24 (253-284) (SEQ ID NO: 179), and Pol 325-355 (RT 158-188) (SEQ ID NO: 180). See Fundamental Virology, Second Edition, eds. Fields, B. N. and Knipe, D. M. (Raven Press, New York, 1991) for additional examples of viral antigens.

Significant progress has been made in genetically engineering T cells of the immune system to target and kill unwanted cell types, such as cancer cells. Many of these T cells have been genetically engineered to express chimeric antigen receptor (CAR) constructs. CARs are proteins including several distinct subcomponents that allow the genetically modified T cells to recognize and kill cancer cells. The subcomponents include at least an extracellular component and an intracellular component.

The extracellular component includes a binding domain that specifically binds a marker that is preferentially present on the surface of unwanted cells. When the binding domain binds such markers, the intracellular component directs the T cell to destroy the bound cancer cell. The binding domain is typically a single-chain variable fragment (scFv) derived from a monoclonal antibody (mAb), but it can be based on other formats which include an antibody-like antigen binding site.

The intracellular components provide activation signals based on the inclusion of an effector domain. First generation CARs utilized the cytoplasmic region of CD3 as an effector domain. Second generation CARs utilized CD3 in combination with cluster of differentiation 28 (CD28) or 4-1 BB (CD137), while third generation CARs have utilized CD3 in combination with CD28 and 401BB within intracellular effector domains.

CAR generally also include one or more linker sequences that are used for a variety of purposes within the molecule. For example, a transmembrane domain can be used to link the extracellular component of the CAR to the intracellular component. A flexible linker sequence often referred to as a spacer region that is membrane-proximal to the binding domain can be used to create additional distance between a binding domain and the cellular membrane. This can be beneficial to reduce steric hindrance to binding based on proximity to the membrane. A common spacer region used for this purpose is the IgG4 linker. More compact spacers or longer spacers can be used, depending on the targeted cell marker. Other potential CAR subcomponents are described in more detail elsewhere herein. Components of CAR are now described in additional detail as follows: (a) Binding Domains; (b) Intracellular Signalling Components; (c) Linkers; (d) Transmembrane Domains; (e) Junction Amino Acids; and (f) Control Features Including Tag Cassettes.

(a) Binding Domains. Binding domains include any substance that binds to a cellular marker to form a complex, including without limitation all binding domains and antibodies disclosed herein. The choice of binding domain can depend upon the type and number of cellular markers that define the surface of a target cell. Examples of binding domains include cellular marker ligands, receptor ligands, antibodies, peptides, peptide aptamers, receptors (e.g., T cell receptors), or combinations and engineered fragments or formats thereof.

(b) Intracellular Signaling Components. The intracellular or otherwise the cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-IBB (CD137), CARD11, CD3γ, CD35, CD3E, CD3 CD27, CD28, CD79A, CD79B, DAP10, FcRα, FcR8 (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRa, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8a, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAMI, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3 retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CD5, ICAM-1, GITR, BAFFR, HVEM (LIGHTR), SLAMF7, NKp80 (KLRF1), NKp44, NKp30, NKp46, CD160, CD19, CD4, CD8a, CD8β, IL2Rβ, IL2Rγ, IL7Rα, ITGA4, VLA1, CD49a, ITGA4, IA4, CD49D, ITGA6, VLA-6, CD49f, ITGAD, CDlld, ITGAE, CD103, ITGAL, CDlla, ITGAM, CDI Ib, ITGAX, CDllc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAMI, CRTAM, Ly9 (CD229), PSGL1, CD100 (SEMA4D), CD69, SLAMF6 (NTB-A, Lyl08), SLAM (SLAMFI, CD150, IPO-3), BLAME (SLAMF8), SELPLG (CD162), LTBR, LAT, GADS, SLP-76, PAG/Cbp, and CD19a.

In particular embodiments, the amino acid sequence of the intracellular signaling component includes a variant of CD3 and a portion of the 4-1 BB intracellular signaling component.

In particular embodiments, the intracellular signaling component includes (i) all or a portion of the signaling domain of CD3, (ii) all or a portion of the signaling domain of 4-1BB, or (iii) all or a portion of the signaling domain of CD3 and 4-1BB.

Intracellular components may also include one or more of a protein of a Wnt signaling pathway (e.g., LRP, Ryk, or ROR2), NOTCH signaling pathway (e.g., NOTCHI, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(c) Linkers. As used herein, a linker can be any portion of a CAR molecule that serves to connect two other subcomponents of the molecule. Some linkers serve no purpose other than to link other components while many linkers serve an additional purpose. Linkers in the context of linking VL and VH of antibody derived binding domains of scFv are described above. Linkers can also include spacer regions, and junction amino acids.

Spacer regions are a type of linker region that are used to create appropriate distances and/or flexibility from other linked components. In particular embodiments, the length of a spacer region can be customized for individual cellular markers on unwanted cells to optimize unwanted cell recognition and destruction. The spacer can be of a length that provides for increased responsiveness of the cell following antigen binding, as compared to in the absence of the spacer. In particular embodiments, a spacer region length can be selected based upon the location of a cellular marker epitope, affinity of a binding domain for the epitope, and/or the ability of the modified cells expressing the molecule to proliferate in vitro and/or in vivo in response to cellular marker recognition. Spacer regions can also allow for high expression levels in modified cells.

Exemplary spacers include those having 10 to 250 amino acids, 10 to 200 amino acids, 10 to 150 amino acids, 10 to 100 amino acids, 10 to 50 amino acids, or 10 to 25 amino acids. In particular embodiments, a spacer region is 12 amino acids, 20 amino acids, 21 amino acids, 26 amino acids, 27 amino acids, 45 amino acids, or 50 amino acids.

In particular embodiments, the spacer region is selected from the group including all or a portion of a hinge region sequence from IgG1, IgG2, IgG3, IgG4 or IgD alone or in combination with all or a portion of a CH2 region; all or a portion of a CH3 region; or all or a portion of a CH2 region and all or a portion of a CH3 region.

Exemplary spacers include IgG4 hinge alone, IgG4 hinge linked to CH2 and CH3 domains, or IgG4 hinge linked to the CH3 domain. In particular embodiments, the spacer includes an IgG4 linker of the amino acid sequence SEQ ID NO: 181. Hinge regions can be modified to avoid undesirable structural interactions such as dimerization with unintended partners.

In particular embodiments, a spacer region includes a hinge region that a type II C-lectin interdomain (stalk) region or a cluster of differentiation (CD) molecule stalk region. As used herein, a “wild type immunoglobulin hinge region” refers to a naturally occurring upper and middle hinge amino acid sequences interposed between and connecting the CHI and CH2 domains (for IgG, IgA, and IgD) or interposed between and connecting the CHI and CH3 domains (for IgE and IgM) found in the heavy chain of an antibody.

A “stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al, Immunity 10:15, 1999; for descriptions of other stalk regions, see also Beavil et al, Proc. Nat'l. Acad. Sci. USA 89:153, 1992; and Figdor et al, Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

Exemplary spacers also include those described in Hudecek et al. (Clin. Cancer Res., 19:3153, 2013) or WO2014/031687. In particular embodiments, the spacer region can be a CD28 linker of the amino acid sequence SEQ ID NO: 182. In particular embodiments, the spacer region is SEQ ID NO: 183. In particular embodiments, the spacer region is SEQ ID NO: 184.

In particular embodiments, a long spacer is greater than 119 amino acids (e.g., 229 amino acids) an intermediate spacer is 13-119 amino acids, and a short spacer is 12 amino acids or less. An example of an intermediate spacer region includes all or a portion of a IgG4 hinge region sequence and a CH3 region. An example of a long spacer includes all or a portion of a IgG4 hinge region sequence, a CH2 region, and a CH3 region. In particular embodiments of the present disclosure, short spacer sequences are preferred.

As further description regarding spacer regions, an extracellular component of a fusion protein optionally includes an extracellular, non-signaling spacer or linker region, which, for example, can position the binding domain away from the host cell (e.g., T cell) surface to enable proper cell/cell contact, antigen binding and activation (Patel et al., Gene Therapy 6: 412-419 (1999)). As indicated, an extracellular spacer region of a fusion binding protein is generally located between a hydrophobic portion or transmembrane domain and the extracellular binding domain, and the spacer region length may be varied to maximize antigen recognition (e.g., tumor recognition) based on the selected target molecule, selected binding epitope, or antigen-binding domain size and affinity (see, e.g., Guest et al., J. Immunother. 28:203-11, 2005; WO 2014/031687). In certain embodiments, a spacer region includes an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. An exemplary altered IgG4 hinge region is described in PCT Publication No. WO 2014/031687. Other examples of hinge regions used in the fusion binding proteins described herein include the hinge region present in the extracellular regions of type 1 membrane proteins, such as CD8a, CD4, CD28 and CD7, which may be wild-type or variants thereof.

In certain embodiments, an extracellular spacer region includes all ora portion of an Fc domain selected from: a CHI domain, a CH2 domain, a CH3 domain, a CH4 domain, or any combination thereof (see, e.g., WO 2014/031687). The Fc domain or portion thereof may be wildtype of altered (e.g., to reduce antibody effector function). In certain embodiments, the extracellular component includes an immunoglobulin hinge region, a CH2 domain, a CH3 domain, or any combination thereof disposed between the binding domain and the hydrophobic portion. In certain embodiments, the extracellular component includes an IgG1 hinge region, an IgG1 CH2 domain, and an IgG1 CH3 domain. In further embodiments, the IgG1 CH2 domain includes (i) a N297Q mutation, (ii) substitution of the first six amino acids (APEFLG) with APPVA, or both of (i) and (ii). In certain embodiments, the immunoglobulin hinge region, Fc domain or portion thereof, or both are human.

(d) Transmembrane Domains. As indicated, transmembrane domains within a CAR molecule, often serving to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-IBB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDIIa, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAMI, CRT AM, Ly9(CD229), PSGL1, CD100 (SEMA4D), SLAMF6 (NTB-A, Lyl08), SLAM (SLAMFI, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell. In particular embodiments, the transmembrane domain includes the amino acid sequence of the CD28 transmembrane domain.

(e) Junction Amino Acids. Junction amino acids can be a linker which can be used to connect the sequences of CAR domains when the distance provided by a spacer is not needed and/or wanted. Junction amino acids are short amino acid sequences that can be used to connect co-stimulatory intracellular signaling components. In particular embodiments, junction amino acids are 9 amino acids or less.

Junction amino acids can be a short oligo- or protein linker, preferably between 2 and 9 amino acids (e.g., 2, 3, 4, 5, 6, 7, 8, or 9 amino acids) in length to form the linker. In particular embodiments, a glycine-serine doublet can be used as a suitable junction amino acid linker. In particular embodiments, a single amino acid, e.g., an alanine, a glycine, can be used as a suitable junction amino acid.

(f) Control Features Including Tag Cassettes, Transduction Markers, and Suicide Switches. In particular embodiments, CAR constructs can include one or more tag cassettes, transduction markers, and/or suicide switches. In some embodiments, the transduction marker and/or suicide switch is within the same construct but is expressed as a separate molecule on the cell surface. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a CAR, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CAR molecule.

Tag cassettes that bind cognate binding molecules include, for example, His tag, Flag tag, Xpress tag, Avi tag, Calmodulin tag, Polyglutamate tag, HA tag, Myc tag, Softag 1, Softag 3, and V5 tag. In particular embodiments, a CAR includes a Myc tag.

Conjugate binding molecules that specifically bind tag cassette sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal.

Transduction markers may be selected from at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al., Mol. Therapy 1(5 Pt 1); 448-456, 2000) and CD20 antigens (see Philip et al., Blood 124: 1277-1278, 2014).

In particular embodiments, a polynucleotide encoding an iCaspase9 construct (iCasp9) may be inserted into a CAR nucleotide construct as a suicide switch.

Control features may be present in multiple copies in a CAR or can be expressed as distinct molecules with the use of a skipping element. For example, a CAR can have one, two, three, four or five tag cassettes and/or one, two, three, four, or five transduction markers could also be expressed. For example, embodiments can include a CAR construct having two Myc tag cassettes, or a His tag and an HA tag cassette, or a HA tag and a Softag 1 tag cassette, or a Myc tag and a SBP tag cassette. In particular embodiments, CAR that will multimerize following expression include different tag cassettes. In particular embodiments, a transduction marker includes tEFGR. Exemplary transduction markers and cognate pairs are described in U.S. Ser. No. 13/463,247.

One advantage of including at least one control feature in a CAR is that CAR expressing cells administered to a subject can be depleted using the cognate binding molecule to a tag cassette. In certain embodiments, the present disclosure provides a method for depleting a modified cell expressing a CAR by using an antibody specific for the tag cassette, using an cognate binding molecule specific for the control feature, or by using a second modified cell expressing a CAR and having specificity for the control feature. Elimination of modified cells may be accomplished using depletion agents specific for a control feature.

In certain embodiments, modified cells expressing a chimeric molecule may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with a CAR can be, e.g., more readily identified, isolated, sorted, induced to proliferate, tracked, and/or eliminated as compared to a modified cell without a tag cassette.

Exemplary CARs and CAR architectures useful in the methods and compositions of the present disclosure include those provided by WO2012/138475A1, U.S. Pat. No. 9,624,306B2, U.S. Pat. No. 9,266,960B2, US2017/017477, EP2694549B1, US2017/0283504, US2017/0281766, US20170283500, US2018/0086846, US2010/0105136, US2010/0105136, WO2012/079000, WO2008045437, WO2016/139487A1, and WO2014/039523.

TCR refer to naturally occurring T cell receptors. HSC can be modified in vivo to express a selected TCR. CAR/TCR hybrids refer to proteins having an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

Particular CAR/TCR hybrids include TRuC® (T Cell Receptor Fusion Construct) hybrids; TCR2 Therapeutics, Cambridge, Mass. By way of example, the production of TCR fusion proteins is described in International Patent Publications WO 2018/026953 and WO 2018/067993, and in Application Publication US 2017/0166622.

In particular embodiments, CAR/TCR hybrids include a “T-cell receptor (TCR) fusion protein” or “TFP”. A TFP includes a recombinant polypeptide derived from the various polypeptides including the TCR that is generally capable of i) binding to a surface antigen on target cells and ii) interacting with other polypeptide components of the intact TCR complex, typically when co-located in or on the surface of a T-cell.

In particular embodiments, a TFP includes an antibody fragment that binds a cancer antigen (e.g., CD19, ROR1) wherein the sequence of the antibody fragment is contiguous with and in the same reading frame as a nucleic acid sequence encoding a TCR subunit or portion thereof. The TFPs are able to associate with one or more endogenous (or alternatively, one or more exogenous, or a combination of endogenous and exogenous) TCR subunits in order to form a functional TCR complex.

### I(C)(i)(b). Gene Editing Systems and Components

In various embodiments, a payload of the present disclosure encodes at least one component, or all components, of a gene editing system. Gene editing systems of the present disclosure include CRISPR systems and base editing systems. Broadly, gene editing systems can include a plurality of components including a gene editing enzyme selected from a CRISPR-associated RNA-guided endonuclease and a base editing enzyme and at least one gRNA. Accordingly, gene editing systems of the present disclosure can include either (i) in the case of a CRISPR system, a CRISPR enzyme that is a CRISPR-associated RNA-guided endonuclease and at least one guide RNA (gRNA), or (ii) in the case of a base editing system, a base editing enzyme and at least one gRNA.

The present disclosure includes that self-inactivating gene editing systems include gene editing systems that are present in a vector of the present disclosure and are rendered non-functional upon excision and/or integration into a host cell genome of a portion of the vector, e.g., an integration element. In various embodiments, the gene editing system is rendered non-functional by degradation of the vector sequence encoding at least one component of the gene editing system following excision of the integration element and/or integration of the integration element into a host cell genome.

The present disclosure includes, in various embodiments, a nucleic acid sequence encoding a gene editing system in which a CRISPR enzyme or base editing enzyme is operably linked with a PGK promoter. The present disclosure includes the experimental discovery that PGK is a weaker promoter in producer cells such as HEK293 cells for donor vector production (i.e., drives relatively low or reduced levels of coding sequence expression, e.g., as compared to an Ef1α promoter in a producer cell and/or as compared to a PGK promoter in an HSC) but drives efficient transgene expression in HSCs (i.e., drives relatively high or increased levels of coding sequence expression, e.g., as compared to an Ef1α promoter in an HSC and/or as compared to a PGK promoter in a producer cell such as a HEK293 cell).

In various embodiments, a nucleic acid sequence encoding a gene editing system that includes a CRISPR enzyme or base editing enzyme includes a microRNA target site that reduces or suppresses expression of the enzyme in producer cells such as HEK293 cells, e.g., to avoid or reduce potential adverse effects of gene editing system expression (e.g., base editing system expression) in the producer cell(s), e.g., from expression of TadA and/or Tad*. In various embodiments, a miR sequence can be a sequence that suppresses base editing or CRISPR enzyme expression in a producer cell during HDAd35 donor vector production, e.g., as described in Saydaminova et al., Mol. Ther. Meth. Clin. Dev. 1: 14057, 2015; Li et al., Mol. Ther. Meth. Clin. Dev. 9: 390-401, 2018, which are herein incorporated by reference.

For the avoidance of doubt, the present disclosure therefore includes embodiments in which a nucleic acid sequence encoding a gene editing system can include any or all of (i) a nucleic acid sequence encoding a CRISPR enzyme or base editing enzyme, optionally where the nucleic acid sequence includes a modified TadA and/or TadA* as disclosed herein; (ii) a PGK promoter operably linked to the CRISPR enzyme or base editing enzyme coding sequence; and (iii) a microRNA target site that reduces or suppresses expression of the enzyme in producer cells such as HEK293 cells. The present disclosure includes that these features (i, ii, and iii) can contribute to effective gene therapy individually and in synergistic combination.

### I(C)(i)(b)(1). CRISPR Payload Expression Products

The CRISPR (Clustered Regularly Interspaced Short Palindromic Repeats)/Cas (CRISPR-associated protein) nuclease system is an engineered nuclease system used for genetic engineering that is based on a bacterial system. It is based in part on the adaptive immune response of many bacteria and archaea. When a virus or plasmid invades a bacterium, segments of the invaders DNA are converted into CRISPR RNAs (crRNA) by the bacteria's “immune” response. The crRNA then associates, through a region of partial complementarity, with another type of RNA called tracrRNA to guide a Cas nuclease to a region homologous to the crRNA in the target DNA called a “protospacer.” The Cas nuclease cleaves the DNA to generate blunt ends at the double-strand break at sites specified by a 20-nucleotide complementary strand sequence contained within the crRNA transcript. In some instances, the Cas nuclease requires both the crRNA and the tracrRNA for site-specific DNA recognition and cleavage.

Guide RNA (gRNA) is one example of a targeting element. In its simplest form, gRNA provides a sequence that targets a site within a genome based on complementarity (e.g., crRNA). As explained below, however, gRNA can also include additional components. For example, in particular embodiments, gRNA can include a targeting sequence (e.g., crRNA) and a component to link the targeting sequence to a cutting element. This linking component can be tracrRNA. In particular embodiments, as described below, gRNA including crRNA and tracrRNA can be expressed as a single molecule referred to as single gRNA (sgRNA). gRNA can also be linked to a cutting element through other mechanisms such as through a nanoparticle or through expression or construction of a dual or multi-purpose molecule. Those of skill in the art will appreciate that gRNA or other targeting elements to generate a selected nucleic acid sequence correction or modification, e.g., in a host cell of an adenoviral donor vector or genome of the present disclosure, can be readily designed and implemented, e.g., based on available sequence information.

In particular embodiments, targeting elements (e.g., gRNA) can include one or more modifications (e.g., a base modification, a backbone modification), to provide the nucleic acid with a new or enhanced feature (e.g., improved stability). Modified backbones may include those that retain a phosphorus atom in the backbone and those that do not have a phosphorus atom in the backbone. Suitable modified backbones containing a phosphorus atom may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable targeting elements having inverted polarity can include a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

Targeting elements can include one or more phosphorothioate and/or heteroatom internucleoside linkages, in particular —CH₂—NH—O—CH₂—, —CH₂—N(CH₃)—O—CH₂— (i.e. a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—).

In particular embodiments, targeting elements can include a morpholino backbone structure. For example, the targeting elements can include a 6-membered morpholino ring in place of a ribose ring. In some of these embodiments, a phosphorodiamidate or other non-phosphodiester internucleoside linkage replaces a phosphodiester linkage.

In particular embodiments, targeting elements can include one or more substituted sugar moieties. Suitable polynucleotides can include a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH₂)nO) mCH₃, O(CH₂)nOCH₃, O(CH₂)nNH₂, O(CH₂)nCH₃, O(CH₂)nONH₂, and O(CH₂)nON((CH₂)nCH₃)₂, where n and m are from 1 to 10.

Examples of cutting elements include nucleases. CRISPR-Cas loci have more than 50 gene families and there are no strictly universal genes, indicating fast evolution and extreme diversity of loci architecture. Exemplary Cas nucleases include Casl, CasIB, Cas2, Cas3, Cas4, Cas5, Cas6, Cas7, Cas8, Cas9 (also known as Csn1 and Csx12), CasIO, Cpfl, C2c3, C2c2 and C2clCsyl, Csy2, Csy3, Csel, Cse2, Cscl, Csc2, Csa5, Csn2, Csm2, Csm3, Csm4, Csm5, Csm6, Cmr1, Cmr3, Cmr4, Cmr5, Cmr6, Cpfl, Csbl, Csb2, Csb3, Csxl7, Csxl4, Csxl0, Csxl6, CsaX, Csx3, Csxl, Csxl5, Csf1, Csf2, Csf3, and Csf4.

There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40W:58-66). Type II Cas nucleases include Casl, Cas2, Csn2, and Cas9. These Cas nucleases are known to those skilled in the art. For example, the amino acid sequence of the Streptococcus pyogenes wild-type Cas9 polypeptide is set forth, e.g., in NCBI Ref. Seq. No. NP 269215, and the amino acid sequence of Streptococcus thermophilus wild-type Cas9 polypeptide is set forth, e.g., in NCBI Ref. Seq. No. WP_011681470.

In particular embodiments, Cas9 refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, includes one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species.

As indicated previously, the CRISPR/Cas system has been engineered such that, in certain cases, crRNA and tracrRNA can be combined into one molecule called a single gRNA (sgRNA). In this engineered approach, the sgRNA guides Cas to target any desired sequence (see, e.g., Jinek et al., Science 337:816-821, 2012; Jinek et al., eLife 2:e00471, 2013; Segal, eLife 2:e00563, 2013). Thus, the CRISPR/Cas system can be engineered to create a double-strand break at a desired target in a genome of a cell, and harness the cell's endogenous mechanisms to repair the induced break by HDR, or NHEJ. Particular embodiments described herein utilize homology arms to promote HDR at defined integration sites.

Useful variants of the Cas9 nuclease include a single inactive catalytic domain, such as a RuvC″ or HNH″ enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include N854A and N863 A. A double-strand break is introduced using a Cas9 nickase if at least two DNA-targeting RNAs that target opposite DNA strands are used. A double-nicked induced double-strand break is repaired by HDR or NHEJ. This gene editing strategy generally favors HDR and decreases the frequency of indel mutations at off-target DNA sites. The Cas9 nuclease or nickase, in some embodiments, is codon-optimized for the target cell or target organism.

Particular embodiments can utilize Staphylococcus aureus Cas9 (SaCas9). Particular embodiments can utilize SaCas9 with mutations at one or more of the following positions: E782, N968, and/or R1015. Particular embodiments can utilize SaCas9 with mutations at one or more of the following positions: E735, E782, K929, N968, A1021, K1044 and/or R1015. In some embodiments, the variant SaCas9 protein includes one or more of the following mutations: R1015Q, R1015H, E782K, N968K, E735K, K929R, A1021T, and/or K1044N. In some embodiments, the variant SaCas9 protein includes mutations at D10A, D556A, H557A, N580A, e.g., D10A/H557A and/or D10A/D556A/H557A/N580A. In some embodiments, the variant SaCas9 protein includes one or more mutations selected from E735, E782, K929, N968, R1015, A1021, and/or K1044. In some embodiments, the SaCas9 variants can include one of the following sets of mutations: E782K/N968K/R1015H (KKH variant); E782K/K929R/R1015H (KRH variant); or E782K/K929R/N968K/R1015H (KRKH variant).

A Class II, Type V CRISPR-Cas class exemplified by Cpf1 has been identified Zetsche et al. (2015) Cell 163(3): 759-771. The Cpf1 nuclease particularly can provide added flexibility in target site selection by means of a short, three base pair recognition sequence (TTN), known as the protospacer-adjacent motif or PAM. Cpf1's cut site is at least 18 bp away from the PAM sequence. Moreover, staggered DSBs with sticky ends permit orientation-specific donor template insertion, which is advantageous in non-dividing cells.

Particular embodiments can utilize engineered Cpfls. For example, US 2018/0030425 describes engineered Cpf1 nucleases from Lachnospiraceae bacterium ND2006 and Acidaminococcus sp. BV3L6 with altered and improved target specificity. Particular variants include Lachnospiraceae bacterium ND2006, e.g., at least including amino acids 19-1246 with mutations (i.e., replacement of the native amino acid with a different amino acid, e.g., alanine, glycine, or serine), at one or more of the following positions: S202, N274, N278, K290, K367, K532, K609, K915, Q962, K963, K966, K1002, and/or S1003. Particular Cpf1 variants can also include Acidaminococcus sp. BV3L6 Cpf1 (AsCpf1) with mutations (i.e., replacement of the native amino acid with a different amino acid, e.g., alanine, glycine, or serine (except where the native amino acid is serine)), at one or more of the following positions: N178, S186, N278, N282, R301, T315, S376, N515, K523, K524, K603, K965, Q1013, Q1014, and/or K1054.

Other Cpf1 variants include Cpf1 homologs and orthologs of the Cpf1 polypeptides disclosed in Zetsche et al. (2015) Cell 163: 759-771 as well as the Cpf1 polypeptides disclosed in U.S. 2016/0208243. Other engineered Cpf1 variants are known to those of ordinary skill in the art and included within the scope of the current disclosure (see, e.g., WO/2017/184768).

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016/205711, WO2017/106657, WO2017/127807 and applications related thereto.

In some embodiments a CRISPR system is engineered to modify a nucleic acid sequence that encodes γ-globin, e.g., to increase expression of γ-globin. The main fetal form of hemoglobin, hemoglobin F (HbF) is formed by pairing of γ-globin polypeptide subunits with α-globin polypeptide subunits. Human fetal γ-globin genes (HBG1 and HBG2; two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficially, particularly in subjects with β-globin deficiency. A variety of mutations that cause increased expression of γ-globin are known in the art or disclosed herein (see, e.g., Wienert, Trends in Genetics 34(12): 927-940,2018, which is incorporated herein by reference in its entirety and with respect to mutations that increase expression of γ-globin). Certain such mutations are found in the HBG1 promoter or HBG2 promoter.

In some embodiments, a vector or genome includes a CRISPR system in which a payload includes an integration element and at least one component of the CRISPR system is present in the payload but outside of the integration element (e.g., outside of the fragment of a payload including a transposable integration element that is flanked by the transposon inverted repeats or outside of the fragment of a payload that includes homology arms for homologous integration). In certain particular embodiments in which a payload includes a transposable integration element, where the transposable integration element is flanked by transposon inverted repeats, one or more of a CRISPR enzyme and/or one or more gRNAs of the CRISPR system are present in the payload at a position outside of (i.e., not present in) the transposable integration element (i.e., not present in the nucleic acid sequence flanked by the transposon inverted repeats). In certain particular embodiments in which a payload includes a transposable integration element, where the transposable integration element is flanked by homology arms, one or more of a CRISPR enzyme and/or one or more gRNAs of the CRISPR editing system are present in the payload at a position outside of (i.e., not present in) the integration element (i.e., not present in the nucleic acid sequence flanked by the homology arms). In such systems, expression and/or activity of the CRISPR system is transient, in that transposition of the transposable integration element can disrupt the vector and reduce or terminate expression of one or more of the CRISPR system components positioned outside of the transposable integration element. Such vectors that include CRISPR systems can sometimes be referred to as “self-inactivating” CRISPR systems or vectors because integration of the integration element (e.g., by transposition or homologous recombination) can inactivate expression and/or activity of the CRISPR system. In various embodiments, a self-inactivating CRISPR system is present in a combination payload.

The present inventors have observed that an adenoviral vector (e.g., an HDAd adenoviral vector) including a self-inactivating CRISPR system payload resulted in an increased cleavage frequency in gene therapy (e.g., in vivo gene therapy) and/or increased survival of transduced and/or edited target cells (e.g., increased survival of transduces HSPCs) as compared to other CRISPR system payloads, e.g., wherein a CRISPR system is fully within an integration element or in which the CRISPR system does not integrate into a host cell genome but expression is not inactivated by vector disruption. Self-inactivation of CRISPR systems shortens expression of the CRISPR enzyme and/or gRNAs, increases survival of edited cells, and increases the percentage of long-term repopulating cells, To provide one example, gene therapy using HDAd vectors including a combination payload including a self-inactivating CRISPR system for reactivation of HBG1 and/or HGB2 and further including a nucleic acid sequence for expression of γ-globin, produced significantly higher γ-globin in RBCs after transduction that did HDAd vectors including either a non-inactivating CRISPR system or nucleic acid sequence for expression of γ-globin alone.

Further provided herein are methods in which a donor vector including a self-inactivating CRISPR system is administered, e.g., to a human subject, in combination with a support vector or genome encoding a transposase for transposition of the integration element. The present disclosure includes that in various instances the donor vector is administered prior to administration of the support vector, wherein the time period between administration of the donor vector and administration of the support vector provides a means of regulating the duration and/or level of activity of the CRISPR system. For instance, in various embodiments, a support vector may be administered, e.g., to a subject, a period of time after administration of the donor vector where the period of time is at least 1, 2, 3, 4, 5, 6, 8, 10, 12, 14, 16, 18, 20, 22, 24, 30, 36, 42, 48, 54, 60, 66, or 72, 96, or 128 hours (e.g., wherein the period has a lower bound of 1, 2, 3, 4, 5, 6, 8, 10, 12, 14, 16, 18, 20, 22, 24, 30, 36, 42, 48, 54, 60, 66, or 72 hours and an upper bound of 6, 8, 10, 12, 14, 16, 18, 20, 22, 24, 30, 36, 42, 48, 54, 60, 66, 72, 96, or 128 hours).

In some embodiments, a nucleic acid sequence encoding a CRISPR system component (e.g., encoding a CRISPR enzyme) is engineered to include a microRNA target site for microRNA regulation of CRISPR expression and/or activity.

### I(C)(i)(b)(2). Base Editor Payload Expression Products

The present disclosure includes, among other things, base editing agents and nucleic acids encoding the same, optionally wherein a base editing agent or nucleic acid encoding the same is present in an vector or genome such as an adenoviral vector or genome. A base editing system can include a base editing enzyme and/or at least one gRNA as components thereof. In certain particular embodiments, a base editing agent and/or a base editing system of the present disclosure is present in an Ad35 or Ad5/35 adenoviral vector. However, those of skill in the art will appreciate that base editing agents of the present disclosure and nucleic acid sequences encoding the same can be present in any context or form, e.g., in a vector that is not an adenoviral vector, e.g., in a plasmid. Nucleotide sequences encoding base editing systems as disclosed herein are typically too large for inclusion in many limited-capacity vector systems, but the large capacity of adenoviral vectors permits inclusion of such sequences in adenoviral vectors and genomes of the present disclosure. Indeed, as discussed elsewhere herein, adenoviral vectors can include payloads that encode a base editing system and further encode one or more additional coding sequences. An additional advantage of adenoviral vectors and genomes as disclosed herein for gene therapy with payloads encoding base editors of the present disclosure is that adenoviral genomes such as Ad35 genomes do not naturally integrate into host cell genomes, which facilitates transient expression of base editing systems, which can be desirable, e.g., to avoid immunogenicity and/or genotoxicity.

Base editing refers to the selective modification of a nucleic acid sequence by converting a base or base pair within genomic DNA or cellular RNA to a different base or base pair (Rees & Liu, Nature Reviews Genetics, 19:770-788, 2018). There are two general classes of DNA base editors: (i) cytosine base editors (CBEs) that convert guanine-cytosine base pairs into thymine-adenine base pairs, and (ii) adenine base editors (ABEs) that convert adenine-thymine base pairs to guanine cytosine base pairs. In particular embodiments, components from the CRISPR system are combined with other enzymes or biologically active fragments thereof to directly install, cause, or generate mutations such as point mutations in nucleic acids, e.g., into DNA or RNA, e.g., without making, causing, or generating one or more double-stranded breaks in the mutated nucleic acid. Certain such combinations of components are known as base editors.

DNA base editors can include a catalytically disabled nuclease fused to a nucleobase deaminase enzyme and, in some cases, a DNA glycosylase inhibitor. RNA base editors achieve analogous changes using components that base modify RNA.

Upon binding to its target locus in DNA, base pairing between the guide RNA and target DNA strand leads to displacement of a small segment of single-stranded DNA. DNA bases within this single-stranded DNA bubble can be modified by the deaminase enzyme. In certain embodiments, to improve efficiency in eukaryotic cells, a catalytically disabled nuclease also generates a nick in the non-edited DNA strand, inducing cells to repair the non-edited strand using the edited strand as a template.

For CBEs, CRISPR-based editors can be produced by linking a cytosine deaminase with a Cas nickase, e.g., Cas9 nickase (nCas9). To provide one example, nCas9 can create a nick in target DNA by cutting a single strand, reducing the likelihood of detrimental indel formation as compared to methods that require a double-stranded break. After binding with DNA, the CBE deaminates a target cytosine (C) into a uracil (U) base. Later the resultant U-G pair is either repaired by cellular mismatch repair machinery making an original C-G pair converted to T-A or reverted to the original C-G by base excision repair mediated by uracil glycosylase. In various embodiments, expression of uracil glycosylase inhibitor (UGI), e.g., a UGI present in a payload, reduces the occurrence of the second outcome and increases the generation of T-A base pair formation.

For adenosine base editors (ABEs), exemplary adenosine deaminases that can act on DNA for adenine base editing include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. As the cellular inosine excision repair is not as active as uracil excision, ABE does not require any additional inhibitor protein like UGI in CBE. In some embodiments, a typical ABE can include three components including a wild-type E. coli tRNA-specific adenosine deaminase (TadA) monomer, which can play a structural role during base editing, a TadA* mutant TadA monomer that catalyzes deoxyadenosine deamination, and a Cas nickase such as Cas9(D10A). In certain embodiments, there is a linker positioned between TadA and TadA*, and in certain embodiments there is a linker positioned between TadA* and the Cas nickase. In various embodiments, one or both linkers includes at least 6 amino acids, e.g., at least 5, 6, 7, 8, 9, 10, 15, 20, 25, 30, 35, 40, 45, or 50 amino acids (e.g., having a lower bound of 5, 6, 7, 8, 9, 10, or 15, amino acids and an upper bound of 20, 25, 30, 35, 40, 45, or 50 amino acids). In various embodiments, one or both linkers include 32 amino acids. In some embodiments, one or both linkers has a sequence according to (SGGS)2-XTEN-(SGGS)2, or a sequence otherwise known to those of skill in the art.

Base editors can directly convert one base or base pair into another, enabling the efficient installation of point mutations in non-dividing cells without generating excess undesired editing by-products, such as insertions and deletions (indels). For example, base editors can generate less than 10%, 9%, 8%, 7%, 6%, 5.5%, 5%, 4.5%, 4%, 3.5%, 3%, 2.5%, 2%, 1.5%, 1%, 0.5%, or 0.1% indels.

DNA base editors can insert such point mutations in non-dividing cells without generating double-strand breaks. Due to the lack of double-strand breaks, base editors do not result in excess undesired editing by-products, such as insertions and deletions (indels). For example, base editors can generate fewer than 10%, 9%, 8%, 7%, 6%, 5.5%, 5%, 4.5%, 4%, 3.5%, 3%, 2.5%, 2%, 1.5%, 1%, 0.5%, or 0.1% indels as compared to technologies that do rely on double-strand breaks.

Components of most base-editing systems include (1) a targeted DNA binding protein, (2) a nucleobase deaminase enzyme, and (3) a DNA glycosylase inhibitor.

Any nuclease of the CRISPR system can be disabled and used within a base editing system. Exemplary Cas nucleases include Casl, CasIB, Cas2, Cas3, Cas4, Cas5, Cas6, Cas7, Cas8, Cas9 (also known as Csnl and Csxl2), CasIO, Cpfl, C2c3, C2c2 and C2clCsyl, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Cpfl, Csbl, Csb2, Csb3, Csxl7, Csxl4, CsxIO, Csxl6, CsaX, Csx3, Csxl, Csxl5, Csf1, Csf2, Csf3, Csf4 and mutations thereof.

Particular embodiments utilize a nuclease-inactive Cas9 (dCas9) as the catalytically disabled nuclease. However, any nuclease of the CRISPR system (many of which are described above) can be disabled and used within a base editing system. In particular embodiments, a Cas9 domain with high fidelity is selected wherein the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver, et al., Nature 529, 490-495, 2016; and Slaymaker et al., Science 351, 84-88, 2015.

Nucleases from other gene-editing systems may also be used. For example, base-editing systems can utilize zinc finger nucleases (ZFNs) (Urnov et al., Nat Rev Genet., 11(9):636-46, 2010) and transcription activator like effector nucleases (TALENs) (Joung et al., Nat Rev Mol Cell Biol. 14(1):49-55, 2013). For additional information regarding DNA-binding nucleases, see US2018/0312825A1.

In particular embodiments, the nucleobase deaminase enzyme includes a cytidine deaminase domain or an adenine deaminase domain.

Particular embodiments utilize a cytidine deaminase domain as the nucleobase deaminase enzyme. Particular embodiments utilize an adenine deaminase domain as the nucleobase deaminase enzyme. Further, particular embodiments utilize a uracil glycosylase inhibitor (UGI) as a glycosylase inhibitor. For example, in particular embodiments, dCas9 or a Cas9 nickase can be fused to a cytidine deaminase domain. The dCas9 or a Cas9 nickase fused to the cytidine deaminase domain can be fused to one or more UGI domains. Base editors with more than one UGI domain can generate less indels and more efficiently deaminates target nucleic acids.

In particular embodiments, a deaminase domain (cytidine and/or adenine) is fused to the N-terminus of the catalytically disabled nuclease. This is because a cytidine deaminase domain fused to the N-terminus of Cas9 can have improved base-editing efficiency when compared to other configurations. In these embodiments, a glycosylase inhibitor (e.g., UGI domain) can be fused to the C-terminus of the catalytically disabled nuclease. When multiple glycosylase inhibitors are used, each can be fused to the C-terminus of the catalytically disabled nuclease.

In particular embodiments, CBE utilizing a cytidine deaminase domain convert guanine-cytosine base pairs into thymine-adenine base pairs by deaminating the exocyclic amine of the cytosine to generate uracil. Examples of cytosine deaminase enzymes include APOBECI, APOBEC3A, APOBEC3G, CDA1, and AID. APOBECI particularly accepts single stranded (ss)DNA as a substrate but is incapable of acting on double stranded (ds)DNA.

Most base-editing systems also include a DNA glycosylase inhibitor that serves to override natural DNA repair mechanisms that might otherwise repair the intended base editing. In particular embodiments, the DNA glycosylase inhibitor includes an uracil glycosylase inhibitor, such as the uracil DNA glycosylase inhibitor protein (UGI) described in Wang et al. (Gene 99, 31-37, 1991).

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester); amino acid linkers; carbon-nitrogen bond amide linkers; cyclic or acyclic, substituted or unsubstituted, branched or unbranched aliphatic or heteroaliphatic linkers; monomeric, dimeric, or polymeric aminoalkanoic acid linkers; aminoalkanoic acid (e.g., glycine, ethanoic acid, alanine, β-alanine, 3-aminopropanoic acid, 4-aminobutanoic acid, 5-pentanoic acid) linkers; monomeric, dimeric, or polymeric aminohexanoic acid (Ahx) linkers; carbocyclic moiety (e.g., cyclopentane, cyclohexane) linkers; aryl or heteroaryl moiety linkers; and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4-100 amino acids in length. In particular embodiments, linkers are 4 amino acids, 9 amino acids, 14 amino acids, 16 amino acids, 32 amino acids, or 100 amino acids.

Numerous base-editing (BE) systems formed by linking targeted DNA binding proteins with cytidine deaminase enzymes and DNA glycosylase inhibitors (e.g., UGI) have been described. These complexes include for example, BEI ([APOBECI-16 amino acid (aa) linker-Sp dCas9 (D10A, H840A)] Korner et al., Nature, 533, 420-424, 2016), BE2 ([APOBECI-16aa linker-Sp dCas9 (D10A, H840A)-4aa linker-UGI] Komer et al., 2016 supra), BE3 ([APOBECI-16aa linker-Sp nCas9 (D10A)-4aa linker-UGI]Korner et al., supra), HF-BE3 ([APOBECI-16aa linker-HF nCas9 (D10A)-4aa linker-UGI] Rees et al., Nat. Commun. 8, 15790, 2017), BE4, BE4max ([APOBECI-32aa linker-Sp nCas9 (D10A)-9aa linker-UGI-9aa linker-UGI] Koblan et al., Nat. Biotechnol 10.1038/nbt.4172, 2018; Komer et al., Sci. Adv., 3, eaao4774, 2017), BE4-GAM ([Gam-16aa linker-APOBECI-32aa linker-Sp nCas9 (D10A)-9aa linker-UGI-9aa linker-UGI] Komer et al., 2017 supra), YE1-BE3 ([APOBECI (W90Y, R126E)-16aa linker-Sp nCas9 (D10A)-4aa linker-UGI] Kim et al., Nat. Biotechnol. 35, 475-480, 2017), EE-BE3 ([APOBECI (R126E, R132E)-16aa linker-Sp nCas9 (D10A)-4aa linker-UGI] Kim et al., 2017 supra), YE2-BE3 ([APOBECI (W90Y, R132E)-16aa linker-Sp nCas9 (D10A)-4aa linker-UGI]Kim et al., 2017 supra), YEE-BE3 ([APOBECI (W90Y, R126E, R132E)-16aa linker-Sp nCas9 (D10A)-4aa linker-UGI] Kim et al., 2017 supra), VQR-BE3 ([APOBECI-16aa linker-Sp VQR nCas9 (D10A)-4aa linker-UGI] Kim et al., 2017 supra), VRER-BE3 ([APOBECI-16aa linker-Sp VRER nCas9 (D10A)-4aa linker-UGI] Kim et al., Nat. Biotechnol. 35, 475-480, 2017), Sa-BE3 ([APOBECI-16aa linker-Sa nCas9 (D10A)-4aa linker-UGI] Kim et al., 2017 supra), SA-BE4 ([APOBECI-32aa linker-Sa nCas9 (D10A)-9aa linker-UGI-9aa linker-UGI] Komer et al., 2017 supra), SaBE4-Gam ([Gam-16aa linker-APOBECI-32aa linker-Sa nCas9 (D10A)-9aa linker-UGI-9aa linker-UGI] Komer et al., 2017 supra), SaKKH-BE3 ([APOBECI-16aa linker-Sa KKH nCas9 (D10A)-4aa linker-UGI] Kim et al., 2017 supra), Cas12a-BE ([APOBECI-16aa linker-dCas12a-14aa linker-UGI], Li et al., Nat. Biotechnol. 36, 324-327, 2018), Target-AID ([Sp nCas9 (D10A)-100aa linker-CDA1-9aa linker-UGI] Nishida et al., Science, 353, 10.1126/science.aaf8729, 2016), Target-AID-NG ([Sp nCas9 (D10A)-NG-100aa linker-CDA1-9aa linker-UGI] Nishimasu et al., Science, 361(6408): 1259-1262, 2018), xBE3 ([APOBECI-16aa linker-xCas9(D10A)-4aa linker-UGI] Hu et al., Nature, 556, 57-63, 2018), eA3A-BE3 ([APOBEC3A (N37G)-16aa linker-Sp nCas9(D10A)-4aa linker-UGI] Gerkhe et al., Nat. Biotechnol., 10.1038/nbt.4199, 2018), A3A-BE3 ([hAPOBEC3A-16aa linker-Sp nCas9(D10A)-4aa linker-UGI] Wang et al., Nat. Biotechnol. 10.1038/nbt.4198, 2018), and BE-PLUS ([10X GCN4-Sp nCas9(D10A)/ScFv-rAPOBEC1-UGI] Jiang et al., Cell. Res, 10.1038/s41422-018-0052-4, 2018). For additional examples of BE complexes, including adenine deaminase base editors, see Rees & Liu Nat. Rev Genet. 19(12): 770-788, 2018.

For additional information regarding base editors, see US2018/0312825A1, WO2018/165629A, Urnov et al., Nat Rev Genet. 11(9):636-46, 2010; Joung et al., Nat Rev Mol Cell Biol. 14(1):49-55, 2013; Charpentier et al., Nature.; 495(7439):50-1, 2013; Seo & Kim, Nature Medicine. 24, 1493-1495, 2018, and Rees & Liii, Nature Reviews Genetics, 19, 770-78, 2018, each of which is incorporated herein by reference in its entirety and with specific respect to base editors. Certain base editor constructs that can be used in various embodiments of the present disclosure are described in Zafra et al., Nat Biotech, 36(9):888-893, 2018, and Koblan et al., Nat Biotech 36(9):843-846, 2018, each of which is incorporated herein by reference in its entirety and with specific respect to base editor constructs.

In some embodiments a base editor system is engineered to modify a nucleic acid sequence that encodes γ-globin, e.g., to increase expression of γ-globin. The main fetal form of hemoglobin, hemoglobin F (HbF) is formed by pairing of γ-globin polypeptides with α-globin polypeptides. Human fetal γ-globin genes (HBG1 and HBG2; two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficially, particularly in subjects with β-globin deficiency. A variety of mutations that cause increased expression of γ-globin are known in the art or disclosed herein (see, e.g., Wienert Trends in Genetics 34(12): 927-940, 2018, which is incorporated herein by reference in its entirety and with respect to mutations that increase expression of γ-globin). Certain such mutations are found in the HBG1 promoter or HBG2 promoter.

In some embodiments, a vector or genome includes a base editing system in which a payload includes an integration element and at least one component of the base editing system is present in the payload but outside of the integration element (e.g., outside of the fragment of a payload including a transposable integration element that is flanked by the transposon inverted repeats or outside of the fragment of a payload that includes homology arms for homologous integration). In certain particular embodiments in which a payload includes a transposable integration element, where the transposable integration element is flanked by transposon inverted repeats, one or more of a base editing enzyme and/or one or more gRNAs of the base editing system are present in the payload at a position outside of (i.e., not present in) the transposable integration element (i.e., not present in the nucleic acid sequence flanked by the transposon inverted repeats). In certain particular embodiments in which a payload includes a transposable integration element, where the transposable integration element is flanked by homology arms, one or more of a base editing enzyme and/or one or more gRNAs of the base editing system are present in the payload at a position outside of (i.e., not present in) the integration element (i.e., not present in the nucleic acid sequence flanked by the homology arms). In such systems, expression and/or activity of the base editing system is transient, in that transposition of the transposable integration element can disrupt the vector and reduce or terminate expression of one or more of the base editing system components positioned outside of the transposable integration element. Such vectors that include base editing systems can sometimes be referred to as “self-inactivating” base editing systems or vectors because integration of the integration element (e.g., by transposition or homologous recombination) can inactivate expression and/or activity of the base editing system. In various embodiments, a self-inactivating base editing system is present in a combination payload.

The present disclosure includes that an adenoviral vector (e.g., an HDAd adenoviral vector) including a self-inactivating base editing system payload can generate an increased cleavage frequency in gene therapy (e.g., in vivo gene therapy) and/or increased survival of transduced and/or edited target cells (e.g., increased survival of transduces HSPCs) as compared to other base editing system payloads, e.g., wherein a base editing system is fully within an integration element or in which the base editing system does not integrate into a host cell genome but expression is not inactivated by vector disruption. Self-inactivation of base editing systems shortens expression of the base editor enzyme and/or gRNAs, increases survival of edited cells, and increases the percentage of long-term repopulating cells, For example, gene therapy using HDAd vectors including a combination payload including a self-inactivating base editing system for reactivation of HBG1 and/or HBG2 and further including a nucleic acid sequence for expression of γ-globin can produce significantly higher γ-globin in RBCs after transduction that HDAd vectors including either a non-inactivating base editing system or nucleic acid sequence for expression of γ-globin alone.

Further provided herein are methods in which a donor vector including a self-inactivating base editing system is administered, e.g., to a human subject, in combination with a support vector or genome encoding a transposase for transposition of the integration element. The present disclosure includes that in various instances the donor vector is administered prior to administration of the support vector, wherein the time period between administration of the donor vector and administration of the support vector provides a means of regulating the duration and/or level of activity of the base editing system. For instance, in various embodiments, a support vector may be administered, e.g., to a subject, a period of time after administration of the donor vector where the period of time is at least 1, 2, 3, 4, 5, 6, 8, 10, 12, 14, 16, 18, 20, 22, 24, 30, 36, 42, 48, 54, 60, 66, or 72, 96, or 128 hours (e.g., wherein the period has a lower bound of 1, 2, 3, 4, 5, 6, 8, 10, 12, 14, 16, 18, 20, 22, 24, 30, 36, 42, 48, 54, 60, 66, or 72 hours and an upper bound of 6, 8, 10, 12, 14, 16, 18, 20, 22, 24, 30, 36, 42, 48, 54, 60, 66, 72, 96, or 128 hours).

In some embodiments, a nucleic acid sequence encoding a base editing system component (e.g., encoding a base editing enzyme) is engineered to include a microRNA target site for microRNA regulation of base editor expression and/or activity.

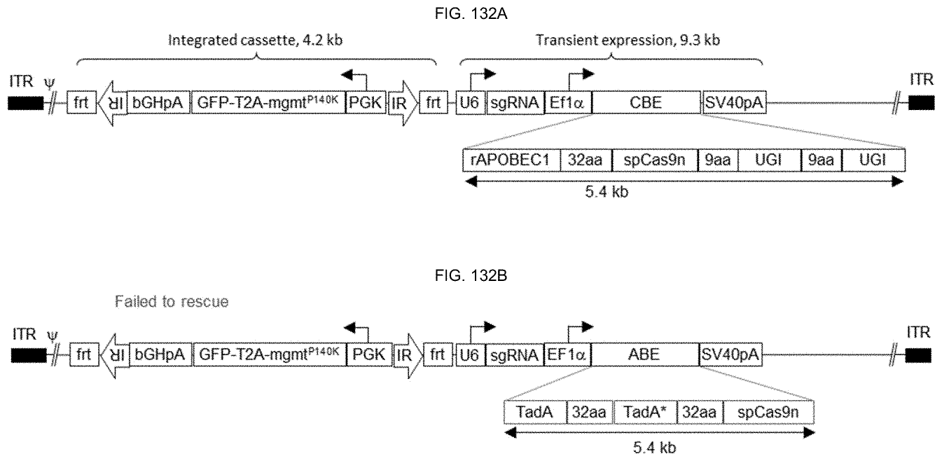

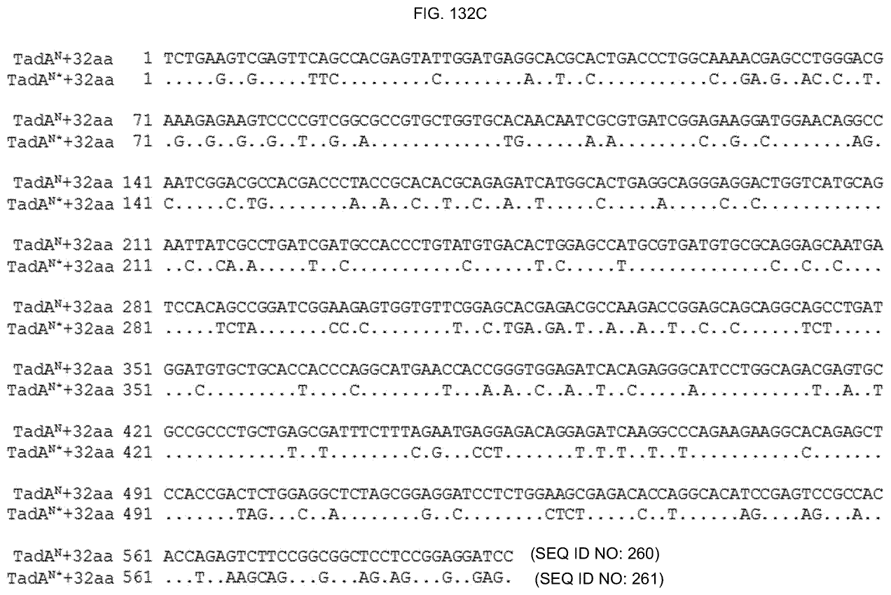

The present disclosure further recognized and solved a problem in the utilization of ABE systems. The present disclosure includes the recognition that repetitiveness and/or sequence similarity in base editor TadA and TadA* sequences can result in homologous recombination that reduces the efficacy of such vectors for expression and/or activity of encoded base editing systems, e.g., for in vivo gene therapy. To the knowledge of the present inventors, the present disclosure represents the first recognition of this problem, e.g., as observed in in vivo gene therapy. To address the problem, TadA and/or TadA* were modified to achieve reduced homology between similar sequences. In various embodiments, at least 5 corresponding codons of nucleic acid sequences encoding TadA and TadA* are engineered to have different nucleotide sequences, optionally wherein the engineering includes replacement of an initial codon sequence in the TadA or TadA* nucleotide sequence with a different codon sequence that encodes the same amino acid according to codon usage in a relevant system, e.g., in humans. In various embodiments, at least 5, 10, 15, 20, 25, 30, 35, 40, 45, or 50 codons are engineered to differ between nucleic acid sequences respectively encoding TadA and TadA*. Exemplary engineered sequences are shown in FIG. 132C.

In various embodiments, an ABE includes TadA and TadA* sequences that include at least one sequence modification relative to the following TadA and TadA* sequences, which can be, e.g., directly fused or separated by a linker in a sequence encoding an ABE. In various embodiments a TadA sequence is a sequence that has at least 80% identity with the below TadA sequence (e.g., at least 80%, 85%, 90%, 95%, 96%, 97%, 98%, 99%, or 100% identity) and can include any or all TadA modifications provided herein. In various embodiments a TadA* sequence is a sequence that has at least 80% identity with the below TadA* sequence (e.g., at least 80%, 85%, 90%, 95%, 96%, 97%, 98%, 99%, or 100% identity) and can include any or all TadA* modifications provided herein. In various embodiments a TadA and/or a TadA* sequence of the present disclosure can include, or not include, a linker such as a 32 amino acid linker. In various sequences and embodiments, including those including the TadA and/or TadA* sequences provided below, a sequence can include a 3′ sequence of 96 nucleotides encoding a 32 amino acid linker. Accordingly, in various embodiments a TadA sequence is a sequence that has at least 80% identity with nucleotides 1-498 (excluding 96 3′ nucleotides) of the below TadA sequence (e.g., at least 80%, 85%, 90%, 95%, 96%, 97%, 98%, 99%, or 100% identity) and can include any or all corresponding TadA modifications provided herein. Also accordingly, in various embodiments a TadA* sequence is a sequence that has at least 80% identity with nucleotides 1-498 (excluding 96 3′ nucleotides) of the below TadA* sequence (e.g., at least 80%, 85%, 90%, 95%, 96%, 97%, 98%, 99%, or 100% identity) and can include any or all corresponding TadA* modifications provided herein.

In various embodiments, the sequence of a TadA and/or a TadA* of an ABE are engineered to reduce the percent identity between the TadA and the TadA* (or an aligned portion thereof, e.g., including nucleotides 1 to 579 or 1 to 498) to less than 80% (e.g., less than 80%, 75%, 70%, 65%, 60%, 55%, 50%, 45%, or 40%, or a percent identity that is between 60% and 80%, 65% and 80%, 70%, and 80%, 75% and 80%, 60% and 75%, 65% and 75%, 70% and 75%, 60% and 70%, or 65% and 70%). In the pCMV-ABEmax plasmid (Addgene #112095) produced by others, there are 109 bp mismatches between the two 594 bp TadA+32aa repeats, having an identity of 81.6%. Sites for TadA and/or TadA* modification in various present embodiments include those underlined in the below sequences and described in the following tables. In various embodiments, a TadA* sequence includes one or more, or all, modifications corresponding to those shown in the TadA* modification table (Table 11). In various embodiments, a TadA sequence includes one or more, or all, modifications shown in the TadA modification table (Table 10) and a TadA* sequence includes one or more, or all, modifications corresponding to those shown in the TadA* modification table (Table 11). In certain particular embodiments, a TadA sequence includes 0, 1, 2, 3, 4, 5, 6, 7, 8. 9. 10, 11, 12, 13, 14, 15, 16, 17, 18, 19, 20, 21, 22, 23, 24, or 25 modifications (e.g., 1 to 5, 5 to 10, 5 to 20, 5 to 25, 10 to 20, 10 to 25, 15 to 20, 15 to 25, or 20 to 25 modifications) corresponding to those shown in the TadA modification table (Table 10; with reference to SEQ ID NO: 280) and a TadA* sequence includes 0, 1, 2, 3, 4, 5, 6, 7, 8. 9. 10, 11, 12, 13, 14, 15, or 16 modifications (e.g., 1 to 5, 5 to 10, 5 to 16, or 10 to 16 modifications) corresponding to those shown in the TadA* modification table (Table 11; with reference to SEQ ID NO: 281).

As those of skill in the art will appreciate, decreased-identity TadA and TadA* sequences are of general utility in the field of genetic engineering, including without limitation in in vivo and ex vivo genetic engineering. TadA and TadA* sequences engineered to have decreased identity can also be included in payloads (e.g., payloads of the present disclosure), e.g., an in adenoviral vector or genome such as an Ad35, Ad35++, HDAd35, or HDAd35++donor vector or donor genome, e.g., for in vivo gene therapy.

TABLE 11
TadA* modification table

| Position | nucleotide change |
|---|---|
| 321 | C > T |
| 330 | C > T |
| 345 | C > T |
| 382 | C > A |
| 384 | C > A |
| 465 | C > T |
| 498 | C > T |
| 499 | T > A |
| 500 | C > G |
| 501 | C > T |
| 504 | A > C |
| 516 | A > G |
| 537 | A > C |
| 592 | T > A |
| 593 | C > G |
| 594 | A > C |

Reference Sequences:
TadA (SEQ ID NO: 280)
TadA* (SEQ ID NO: 281)

TABLE 10
TadA modification table

| Position | nucleotide change |
|---|---|
| 15 | T > C |
| 57 | G > A |
| 63 | A > C |
| 69 | T > C |
| 87 | G > C |
| 112 | A > C |
| 114 | A > C |
| 126 | G > A |
| 147 | C > A |
| 198 | C > A |
| 216 | C > T |
| 289 | A > C |
| 318 | G > A |
| 333 | C > A |
| 343 | T > A |
| 344 | C > G |
| 369 | C > A |
| 402 | A > C |
| 451 | C > A |
| 507 | A > C |
| 547 | A > T |
| 548 | G > C |
| 568 | A > T |
| 569 | G > C |
| 570 | C > T |

Those of skill in the art will further appreciate that the number of modifications corresponding to those of the TadA modification table and/or the TadA* modification table that are present in an ABE including a TadA sequence and a TadA* sequence can be significant without consideration of the particular modifications selected, at least insofar as reduction of the identity between the TadA and TadA* nucleotide sequences is a solution to the identified problem that does not require any particular modification but rather an overall change in the identity between the TadA and TadA* sequences. Thus, while the present disclosure provides exemplary modifications, inclusion or exclusion of any particular modification is not critical to the solution presented herein. The present disclosure therefore includes reduced-identity sequences of TadA and TadA* that include one or more modifications presented in the TadA and TadA* modification tables and have a percent identity between the TadA and the TadA* (or an aligned portion thereof, e.g., including nucleotides 1 to 579) that is less than 80% (e.g., less than 80%, 75%, 70%, 65%, 60%, 55%, 50%, 45%, or 40%).

For the avoidance of doubt, a provided sequence can be identified as including or not including any TadA or TadA* sequence modification provided herein by comparison to a corresponding nucleotide position of the below TadA and TadA* sequences. Accordingly, determination of the presence or absence of any TadA or TadA* sequence modification provided herein does not depend upon the origin or history of any provided sequence and can be determined solely from the sequence itself.

Those of skill in the art will appreciate that ABE systems of the present disclosure, as well as TadA and TadA* sequences thereof, represent contributions of general utility not limited to the present context or any other context set forth in the present specification, e.g., not limited to use in a particular vector, serotype, or other context. Indeed, sequences of the present disclosure can be used in vivo, in vitro, or ex vivo, in any experimental system that can encode or include base editing components. The sequences are useful as tools in various molecular biology applications.

### I(C)(i)(c). Small RNA Payload Expression Products

Small RNAs are short, non-coding RNA molecules that play a role in regulating gene expression. In particular embodiments, small RNAs are less than 200 nucleotides in length. In particular embodiments, small RNAs are less than 100 nucleotides in length. In particular embodiments, small RNAs are less than 50, 45, 40, 35, 30, 25, or 20 nucleotides in length. In particular embodiments, small RNAs are less than 20 nucleotides in length. In various embodiments a small RNA has a length having a lower bound of 5, 10, 15, 20, 25, or 30 nucleotides and an upper bound of 20, 25, 30, 35, 40, 45, 50, 75, or 100 nucleotides. Small RNAs include but are not limited to microRNAs (miRNAs, Piwi-interacting RNAs (piRNAs), small interfering RNAs (siRNAs), small nucleolar RNAs (snoRNAs), tRNA-derived small RNAs (tsRNAs) small rDNA-derived RNAs (srRNAs), and small nuclear RNAs. Additional classes of small RNAs continue to be discovered.

In particular embodiments, interfering RNA molecules that are homologous to a target mRNA or to which the interfering RNA can hybridize can lead to degradation of the target mRNA molecule or reduced translation of the target mRNA, a process referred to as RNA interference (RNAi) (Carthew, Curr. Opin. Cell. Biol. 13: 244-248, 2001). RNAi occurs in cells naturally to remove foreign RNAs (e.g., viral RNAs). In some instances, natural RNAi proceeds via fragments cleaved from free double-strand RNA (dsRNA) which direct the degradative mechanism to other similar RNA sequences. Alternatively, RNAi can be manufactured, for example, to silence the expression of target genes. Exemplary RNAi molecules include small hairpin RNA (shRNA, also referred to as short hairpin RNA) and small interfering RNA (siRNA).

Without limiting the disclosure, and without being bound by theory, RNA interference in nature and/or in some embodiments is typically a two-step process. In the first step, the initiation step, input dsRNA is digested into 21-23 nucleotide (nt) siRNA, probably by the action of Dicer, a member of the ribonuclease (RNase) III family of dsRNA-specific ribonucleases, which processes (cleaves) dsRNA (introduced directly or via a transgene or a virus) in an ATP-dependent manner. Successive cleavage events degrade the RNA to 19-21 base pair (bp) duplexes (siRNA), each with 2-nucleotide 3′ overhangs (Hutvagner & Zamore, Curr. Opin. Genet. Dev. 12: 225-232, 2002; Bernstein, Nature 409:363-366, 2001).

In a second step, an effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and typically cleaves the mRNA into 12 nucleotide fragments from the 3′ terminus of the siRNA (Hutvagner & Zamore, Curr. Opin. Genet. Dev. 12: 225-232, 2002; Hammond et al., Nat. Rev. Gen. 2:110-119, 2001; Sharp, Genes. Dev. 15:485-490, 2001). Research indicates that each RISC contains a single siRNA and an RNase (Hutvagner & Zamore, Curr. Opin. Genet. Dev. 12: 225-232, 2002).

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC (Hutvagner & Zamore, Curr. Opin. Genet. Dev. 12: 225-232, 2002; Hammond et al., Nat. Rev. Gen. 2:110-119, 2001; Sharp, Genes. Dev. 15:485-490, 2001). RNAi is also described in Tuschl (Chem. Biochem. 2: 239-245, 2001); Cullen (Nat. Immunol. 3:597-599, 2002); and Brantl (Biochem. Biophys. Act. 1575:15-25, 2002).

In some embodiments, synthesis of RNAi molecules suitable for use with the present disclosure can be performed as follows. First, an mRNA sequence can be scanned downstream of the start codon of targeted transgene. Occurrence of each AA and the 3′ adjacent 19 nucleotides is recorded as potential siRNA target sites. In particular embodiments, the siRNA target sites can be selected from the open reading frame, as untranslated regions (UTRs) are richer in regulatory protein binding sites. UTR-binding proteins and/or translation initiation complexes may interfere with binding of the siRNA endonuclease complex (Tuschl, Chem. Biochem. 2: 239-245, 2001). It will be appreciated though, that siRNAs directed at untranslated regions may also be effective, as demonstrated for Glyceraldehyde 3-phosphate dehydrogenase (GAPDH) wherein siRNA directed at the 5′ UTR mediated a 90% decrease in cellular GAPDH mRNA and completely abolished protein level. Second, potential target sites can be compared to an appropriate genomic database using any sequence alignment software, such as the Basic Local Alignment Search Tool (BLAST) software available from the National Center for Biotechnology Information (NCBI) server. Putative target sites which exhibit significant homology to other coding sequences can be filtered out.

Qualifying target sequences can be selected as templates for siRNA synthesis. Selected sequences can include those with low G/C content as these have been shown to be more effective in mediating gene silencing as compared to those with G/C content higher than 55%. Several target sites can be selected along the length of the target gene for evaluation. For better evaluation of the selected siRNAs, a negative control can be used. Negative control siRNA can include the same nucleotide composition as the siRNAs but lack significant homology to the genome. Thus, a scrambled nucleotide sequence of the siRNA may be used, provided it does not display any significant homology to other genes.

A sense strand can be designed based on the sequence of the selected portion. The antisense strand is routinely the same length as the sense strand and includes complementary nucleotides. In particular embodiments, the strands are fully complementary and blunt-ended when aligned or annealed. In other embodiments, the strands align or anneal such that 1-, 2- or 3-nucleotide overhangs are generated, i.e., the 3′ end of the sense strand extends 1, 2 or 3 nucleotides further than the 5′ end of the antisense strand and/or the 3′ end of the antisense strand extends 1, 2 or 3 nucleotides further than the 5′ end of the sense strand. Overhangs can include nucleotides corresponding to the target gene sequence (or complement thereof). Alternatively, overhangs can include deoxyribonucleotides, for example deoxythymines (dTs), or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced. In particular embodiments, the base-pair strength is less due to fewer G:C base pairs between the 5′ end of the first or antisense strand and the 3′ end of the second or sense strand than between the 3′ end of the first or antisense strand and the 5′ end of the second or sense strand. In particular embodiments, the base pair strength is less due to at least one mismatched base pair between the 5′ end of the first or antisense strand and the 3′ end of the second or sense strand. Preferably, the mismatched base pair is selected from the group including G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair including a rare nucleotide, e.g., inosine (I). In particular embodiments, the base pair is selected from the group including an I:A, I:U and I:C. In yet another embodiment, the base pair strength is less due to at least one base pair including a modified nucleotide. In particular embodiments, the modified nucleotide is selected from, for example, 2-amino-G, 2-amino-A, 2,6-diamino-G, and 2,6-diamino-A.

ShRNAs are single-stranded polynucleotides with a hairpin loop structure. The single-stranded polynucleotide has a loop segment linking the 3′ end of one strand in the double-stranded region and the 5′ end of the other strand in the double-stranded region. The double-stranded region is formed from a first sequence that is hybridizable to a target sequence, such as a polynucleotide encoding transgene, and a second sequence that is complementary to the first sequence, thus the first and second sequence form a double stranded region to which the linking sequence connects the ends of to form the hairpin loop structure. The first sequence can be hybridizable to any portion of a polynucleotide encoding transgene. The double-stranded stem domain of the shRNA can include a restriction endonuclease site.

Transcription of shRNAs is initiated at a polymerase III (Pol III) promoter and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of 21-23 nucleotides (Brummelkamp et al., Science. 296(5567):550-553, 2002; Lee et al., Nature Biotechnol. 20(5):500-505, 2002; Miyagishi & Taira, Nature Biotechnol. 20(5):497-500, 2002; Paddison et al., Genes & Dev. 16(8): 948-958, 2002; Paul et al., Nature Biotechnol. 20(5):505-508, 2002; Sui, Proc. Natl. Acad. Sci. USA. 99(6):5515-5520, 2002; Yu et al., Proc. Natl. Acad. Sci. USA. 99(9):6047-6052, 2002).

The stem-loop structure of shRNAs can have optional nucleotide overhangs, such as 2-bp overhangs, for example, 3′ UU overhangs. While there may be variation, stems typically range from 15 to 49, 15 to 35, 19 to 35, 21 to 31 bp, or 21 to 29 bp, and the loops can range from 4 to 30 bp, for example, 4 to 23 bp. In particular embodiments, shRNA sequences include 45-65 bp; 50-60 bp; or 51, 52, 53, 54, 55, 56, 57, 58, or 59 bp. In particular embodiments, shRNA sequences include 52 or 55 bp. In particular embodiments siRNAs have 15-25 bp. In particular embodiments siRNAs have 16, 17, 18, 19, 20, 21, 22, 23, or 24 bp. In particular embodiments siRNAs have 19 bp. The skilled artisan will appreciate, however, that siRNAs having a length of less than 16 nucleotides or greater than 24 nucleotides can also function to mediate RNAi. Longer RNAi agents have been demonstrated to elicit an interferon or Protein kinase R (PKR) response in certain mammalian cells which may be undesirable. Preferably the RNAi agents do not elicit a PKR response (i.e., are of a sufficiently short length). However, longer RNAi agents may be useful, for example, in situations where the PKR response has been downregulated or dampened by alternative means.

Small RNAs may also be used to activate gene expression.

### I(C)(i)(d). Combination Payloads

The present disclosure includes adenoviral vectors and genomes in that include a payload that encodes a plurality of expression products. Payloads that encode a plurality of expression products can be referred to as combination payloads. In various embodiments, combination payload can include a first nucleic acid sequence encoding a first expression product and a second nucleic acid sequence encoding a second expression product. In various embodiments, each of the first and second expression products can be independently selected from any of a protein (e.g., a therapeutic protein, e.g., a replacement enzyme), binding domain, antibody, CAR, TCR, CRISPR system, base editor system, a small RNA, and/or a selectable marker e.g., as disclosed herein, Exemplary combination payloads are disclosed herein.

Those of skill in the art will appreciate that coding sequences can be controlled by and/or expressed in operable linkage with any of a variety of promoters and/or other regulatory sequences provided herein or otherwise known in the art. As those of skill in the art will be aware, and as exemplified in the present disclosure, sequences available to control and/or express a coding sequence in a vector are known in the art and include those provided herein. In various particular examples, a coding sequence present in a payload of the present disclosure can be operably linked with one or more regulatory sequences optionally selected from a promoter, enhancer, termination region, insulator, mini-LCR, termination signal, polyadenylation signal, splicing signal, and the like.

In some embodiments, a combination payload encodes one or more, or all, components of a CRISPR system including a CRISPR-associated RNA-guided endonuclease and at least one guide RNA (gRNA), optionally wherein the at least one gRNA include 1, 2, 3, 4, or 5 gRNAs, and optionally one or more further coding sequences not part of the CRISPR system. For example, gRNAs of a CRISPR system can include one or more, or all, of a gRNA that targets a nucleic acid sequence of HBG1 promoter, a gRNA that targets a nucleic acid sequence of HBG2 promoter, and/or a gRNA that targets a nucleic acid sequence of erythroid enhancer bcl11a. In various embodiments, (i) the HBG1 promoter-targeted gRNA is designed to increase expression of a γ-globin coding sequence operably linked to the HBG1 promoter by inactivation of a BCL11A repressor protein binding site in the HBG1 promoter, (ii) the HBG2 promoter-targeted gRNA is designed to increase expression of a γ-globin coding sequence operably linked to the HBG2 promoter by inactivation of a BCL11A repressor protein binding site in the HBG2 promoter, and/or (iii) the bcl11a-targeted gRNA is designed to increase expression of a γ-globin coding sequence operably linked to the bcl11a enhancer, where modification and/or inactivation of the erythroid bcl11a enhancer results in reduced BCL11A repressor protein expression in erythroid cells. In various embodiments, a combination payload that includes a CRISPR system further includes a nucleic acid encoding a therapeutic protein, optionally wherein the therapeutic protein is selected from one or more of γ-globin and β-globin. In some embodiments, the therapeutic protein is operably linked with a β-globin promoter and/or a β-globin LCR.

In some embodiments, a combination payload encodes one or more, or all, components of a base editor system including a base editing enzyme and at least one guide RNA (gRNA), optionally wherein the at least one gRNA include 1, 2, 3, 4, or 5 gRNAs, and optionally one or more further coding sequences not part of the base editor system. For example, gRNAs of a base editor system can include one or more, or all, of a gRNA that targets a nucleic acid sequence of HBG1 promoter, a gRNA that targets a nucleic acid sequence of HBG2 promoter, and/or a gRNA that targets a nucleic acid sequence of erythroid enhancer bcl11a. In various embodiments, (i) the HBG1 promoter-targeted gRNA is designed to increase expression of a γ-globin coding sequence operably linked to the HBG1 promoter by inactivation of a BCL11A repressor protein binding site in the HBG1 promoter, (ii) the HBG2 promoter-targeted gRNA is designed to increase expression of a γ-globin coding sequence operably linked to the HBG2 promoter by inactivation of a BCL11A repressor protein binding site in the HBG2 promoter, and/or (iii) the bcl11a-targeted gRNA is designed to increase expression of a γ-globin coding sequence operably linked to the bcl11a enhancer, where modification and/or inactivation of the erythroid bcl11 a enhancer results in reduced BCL11A repressor protein expression in erythroid cells. In various embodiments, a combination payload that includes a base editor system further includes a nucleic acid encoding a therapeutic protein, optionally wherein the therapeutic protein is selected from one or more of γ-globin and β-globin. In some embodiments, the therapeutic protein is operably linked with a β-globin promoter and/or a β-globin LCR.

In some embodiments, a combination payload includes a nucleic acid sequence that encodes an antibody. In some embodiments a combination payload includes a first nucleic acid sequence that encodes a first antibody and a second nucleic acid sequence that encodes a second antibody. In some embodiments, the antibody (e.g., a first and/or a second antibody) is an scFv. In some embodiments the antibody is an antibody that includes an immunoglobulin heavy chain and an immunoglobulin light chain.

In various embodiments, at least one expression product encoded by a payload nucleic acid sequence of a combination payload is a selectable marker. In various embodiments, the selectable marker is MGMT^{P140K}.

Exemplary Ad35 payloads and systems include:

(i) In various embodiments, an Ad35 payload includes an integration element flanked by transposase inverted repeats for transposition by SB100x, and the transposase inverted repeats are flanked by frt direct repeats for recombination by an FLP recombinase such as FLPe. In various embodiments, the integration element includes, optionally from 5′ to 3′, (a) a β-globin mini-LCR, (b) a gene including a β-globin promoter operably linked with a human γ-globin coding sequence, which γ-globin coding sequence is operably linked with a 3′UTR (e.g., a γ-globin 3′UTR), where the β-globin mini-LCR is also operably linked with the γ-globin coding sequence (c) a cHS4 insulator sequence, and (d) a gene including a promoter such as a PGK promoter operably linked with an MGMTP¹⁴⁰K coding sequence, a 2A self-cleaving peptide, a GFP fluorescent marker coding sequence, and a polyadenylation signal, optionally where any of (a)-(d) can be encoded in a 5′ to 3′ orientation on either of the two strands of an Ad35 payload.

In various embodiments, an Ad35 payload further includes, outside of the integration element and outside of the recombinase sites, a nucleic acid sequence encoding a CRISPR system. In certain particular embodiments, the nucleic acid sequence encoding a CRISPR system includes, optionally from 5′ to 3′, (a) a first gRNA gene including a first U6 promoter operably linked with a first gRNA-encoding sequence, where the first gRNA targets bcl11a enhancer, (b) a second gRNA gene including a second U6 promoter operably linked with a second gRNA-encoding sequence, where the second gRNA targets an HBG promoter, and (c) a CRISPR enzyme gene including a promoter such as an EF1α promoter operably linked with a CRISPR/Cas9 coding sequence, wherein the CRISPR/Cas9 coding sequence is operably linked with a 3′UTR/miR sequence and a polyadenylation signal. In various embodiments, the CRISPR system targets the erythroid bcl11a enhancer and the BCL11A binding site of the HBG promoter, each of which contributes to causing γ-globin activation or re-activation. As disclosed herein, the CRISPR system can be self-inactivating, in that cleavage of donor vector by transposition results in degradation of non-integrated donor vector nucleic acids. In various embodiments, a miR sequence can be a sequence that suppresses Cas9 expression in a producer cell during HDAd35 donor vector production (see, e.g., Saydaminova et al., Mol. Ther. Meth. Clin. Dev. 1: 14057, 2015; Li et al., Mol. Ther. Meth. Clin. Dev. 9: 390-401, 2018).

In various embodiments, an Ad35 system of the present disclosure further includes an Ad35 support vector, where the support vector includes, optionally from 5′ to 3′, (a) a recombines gene including an EF1α promoter operably linked with a FLPe recombinase coding sequence, and (b) a transposase gene including a PGK promoter operably linked with an SB100x transposase coding sequence.

In various embodiments an Ad35 payload is present in an Ad35 donor vector genome. In various embodiments an Ad35 payload present in an Ad35 donor vector genome is flanked by Ad35 ITRs. In various embodiments, an Ad35 donor vector genome is present in an Ad35 donor vector. In various embodiments, the donor vector is an Ad35++ vector.

In various embodiments a support genome includes Ad35 ITRs. In various embodiments, a support genome is present in an Ad35 vector. In various embodiments, the support vector is an Ad35++ vector.

In various embodiments, an Ad35 donor vector is a helper dependent donor vector (HDAd35). In certain such embodiments, systems of the present disclosure can include an HDAd35 donor vector or genome, and Ad35 helper vector or genome, and in various embodiments can further include an Ad35 support vector.

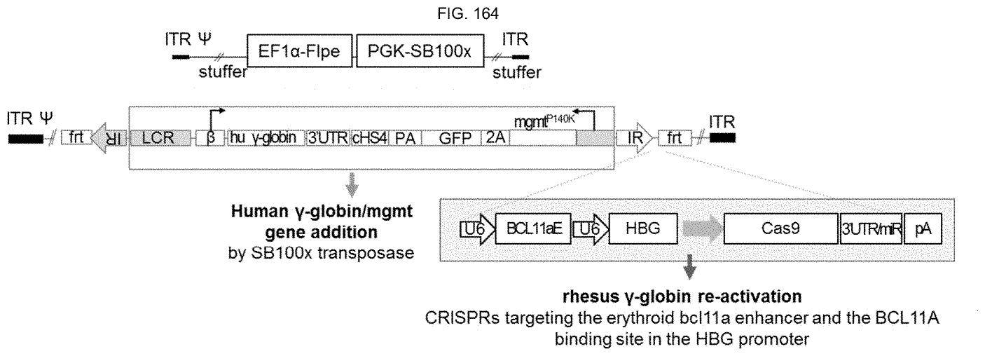

Certain exemplary embodiments are illustrated in FIG. 164.

(ii) In various embodiments, an Ad35 payload includes an integration element flanked by homology arms (e.g., 1.8 kb homology arms), having at least 80% identity (e.g., at least 80%, 85%, 90%, 95%, 96%, 97%, 98%, 99%< or 100% identity) with a target cell genome. In various embodiments, the integration element includes, optionally from 5′ to 3′, (a) a β-globin mini-LCR including HS1, HS2, HS3, and HS4, but not HS5, (b) a gene including a β-globin promoter operably linked with a γ-globin coding sequence, which γ-globin coding sequence is operably linked with a γ-globin 3′UTR, where the β-globin mini-LCR is also operably linked with the γ-globin coding sequence (c) a cHS4 insulator sequence, and (d) a gene including a PGK promoter operably linked with an MGMT^{P140K} coding sequence, where the MGMT^{P140K} coding sequence is operably linked with a polyadenylation signal, optionally where any of (a)-(d) can be encoded in a 5′ to 3′ orientation on either of the two strands of an Ad35 payload.

In various embodiments, an Ad35 payload further includes, outside of the integration element and outside of the recombinase sites, a nucleic acid sequence encoding a CRISPR system. In certain particular embodiments, the nucleic acid sequence encoding a CRISPR system includes, optionally from 5′ to 3′, (a) an sgRNA gene including a U6 promoter operably linked with an sgRNA-encoding sequence, where the sgRNA targets an HBG2 promoter, and (b) a CRISPR enzyme gene including an EF1α promoter operably linked with an spCas9 coding sequence, where the spCas9 coding sequence is operably linked with an miR site, a β-globin 3′UTR sequence, and a polyadenylation signal. In various embodiments, the CRISPR system targets a BCL11A binding site of the HBG promoter and can cause γ-globin activation or re-activation. As disclosed herein, the CRISPR system can be self-inactivating, in that cleavage of donor vector by AAVS1 CRISPR results in degradation of non-integrated donor vector nucleic acids. In various embodiments, a miR sequence can be a sequence that suppresses Cas9 expression in a producer cell during HDAd35 donor vector production (see, e.g., Saydaminova et al., Mol. Ther. Meth. Clin. Dev. 1: 14057, 2015; Li et al., Mol. Ther. Meth. Clin. Dev. 9: 390-401, 2018).

In various embodiments, an Ad35 system of the present disclosure further includes an Ad35 support vector, where the support vector includes, optionally from 5′ to 3′, a U6 promoter operably linked to an sgAAVS1-rm coding sequence.

In various embodiments an Ad35 payload is present in an Ad35 donor vector genome. In various embodiments an Ad35 payload present in an Ad35 donor vector genome is flanked by Ad35 ITRs. In various embodiments, an Ad35 donor vector genome is present in an Ad35 donor vector. In various embodiments, the donor vector is an Ad35++ vector.

In various embodiments a support genome includes Ad35 ITRs. In various embodiments, a support genome is present in an Ad35 vector. In various embodiments, the support vector is an Ad35++ vector.

In various embodiments, an Ad35 donor vector is a helper dependent donor vector (HDAd35). In certain such embodiments, systems of the present disclosure can include an HDAd35 donor vector or genome, and Ad35 helper vector or genome, and in various embodiments can further include an Ad35 support vector.

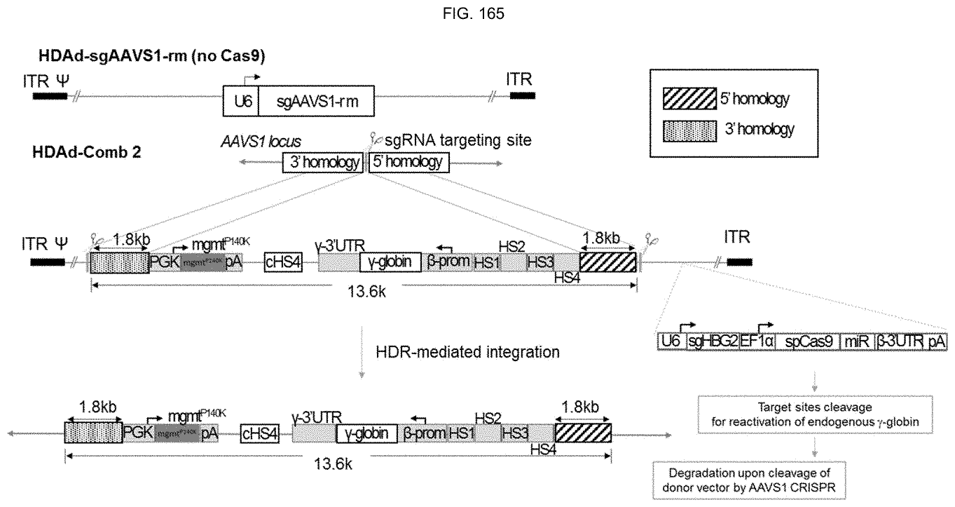

Certain exemplary embodiments are illustrated in FIG. 165.

(iii) In various embodiments, an Ad35 payload includes an integration element flanked by transposase inverted repeats for transposition by SB100x, and the transposase inverted repeats are flanked by frt direct repeats for recombination by an FLP recombinase such as FLPe. In various embodiments, the integration element includes, optionally from 5′ to 3′, (a) a β-globin mini-LCR, (b) a gene including a β-globin promoter operably linked with a rhesus γ-globin coding sequence, which γ-globin coding sequence is operably linked with a 3′UTR (e.g., a γ-globin 3′UTR), where the β-globin mini-LCR is also operably linked with the γ-globin coding sequence (c) a cHS4 insulator sequence, and (d) a gene including a PGK promoter operably linked with an MGMT^{P140K} coding sequence, where the MGMT^{P140K} coding sequence is operably linked with a polyadenylation signal, optionally where any of (a)-(d) can be encoded in a 5′ to 3′ orientation on either of the two strands of an Ad35 payload.

In various embodiments, an Ad35 payload further includes, outside of the integration element and outside of the recombinase sites, a nucleic acid sequence encoding a CRISPR system. In certain particular embodiments, the nucleic acid sequence encoding a CRISPR system includes, optionally from 5′ to 3′, (a) a gRNA gene including a U6 promoter operably linked with a gRNA-encoding sequence, where the gRNA targets an HBG promoter, and (b) a CRISPR enzyme gene including an EF1α promoter operably linked with a CRISPR/Cas9 coding sequence, wherein the CRISPR/Cas9 coding sequence is operably linked with a 3′UTR/miR sequence and a polyadenylation signal. In various embodiments, the CRISPR system targets the BCL11A binding site of the HBG promoter, which can result in γ-globin activation or re-activation. As disclosed herein, the CRISPR system can be self-inactivating, in that cleavage of donor vector by transposition results in degradation of non-integrated donor vector nucleic acids. In various embodiments, a miR sequence can be a sequence that suppresses Cas9 expression in a producer cell during HDAd35 donor vector production (see, e.g., Saydaminova et al., Mol. Ther. Meth. Clin. Dev. 1: 14057, 2015; Li et al., Mol. Ther. Meth. Clin. Dev. 9: 390-401, 2018).

In various embodiments, an Ad35 system of the present disclosure further includes an Ad35 support vector, where the support vector includes, optionally from 5′ to 3′, (a) a recombines gene including an EF1α promoter operably linked with a FLPe recombinase coding sequence, and (b) a transposase gene including a PGK promoter operably linked with an SB100x transposase coding sequence.

In various embodiments an Ad35 payload is present in an Ad35 donor vector genome. In various embodiments an Ad35 payload present in an Ad35 donor vector genome is flanked by Ad35 ITRs. In various embodiments, an Ad35 donor vector genome is present in an Ad35 donor vector. In various embodiments, the donor vector is an Ad35++ vector.

In various embodiments a support genome includes Ad35 ITRs. In various embodiments, a support genome is present in an Ad35 vector. In various embodiments, the support vector is an Ad35++ vector.

In various embodiments, an Ad35 donor vector is a helper dependent donor vector (HDAd35). In certain such embodiments, systems of the present disclosure can include an HDAd35 donor vector or genome, and Ad35 helper vector or genome, and in various embodiments can further include an Ad35 support vector.

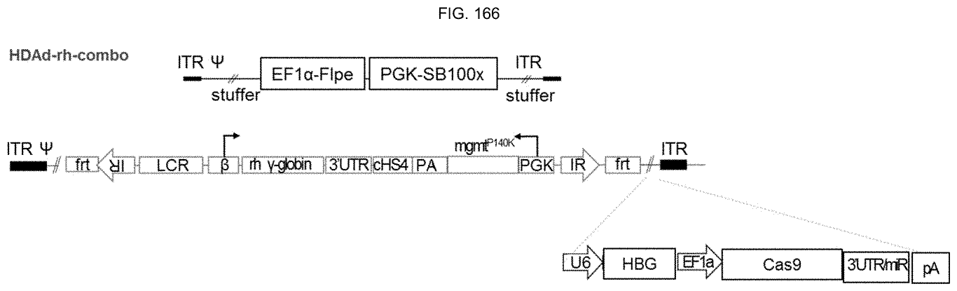

Certain exemplary embodiments are illustrated in FIG. 166.

(iv) In various embodiments, an Ad35 payload includes an integration element flanked by transposase inverted repeats for transposition by SB100x, and the transposase inverted repeats are flanked by frt direct repeats for recombination by an FLP recombinase such as FLPe. In various embodiments, the integration element includes, optionally from 5′ to 3′, (a) a β-globin mini-LCR, (b) a gene including a β-globin promoter operably linked with a human γ-globin coding sequence, which γ-globin coding sequence is operably linked with a 3′UTR (e.g., a γ-globin 3′UTR), where the β-globin mini-LCR is also operably linked with the γ-globin coding sequence (c) a cHS4 insulator sequence, and (d) a gene including a promoter such as a PGK promoter operably linked with an MGMT^{P140K} coding sequence, a 2A self-cleaving peptide, a GFP fluorescent marker coding sequence, and a polyadenylation signal, optionally where any of (a)-(d) can be encoded in a 5′ to 3′ orientation on either of the two strands of an Ad35 payload.

In various embodiments, an Ad35 payload further includes, outside of the integration element and outside of the recombinase sites, a nucleic acid sequence encoding a base editing system. In certain particular embodiments, the nucleic acid sequence encoding a base editing system includes, optionally from 5′ to 3′, (a) a first gRNA gene including a first U6 promoter operably linked with a first gRNA-encoding sequence, where the first gRNA targets bcl11a enhancer, (b) a second gRNA gene including a second U6 promoter operably linked with a second gRNA-encoding sequence, where the second gRNA targets an HBG promoter, and (c) a base editing enzyme gene including a promoter such as an EF1α promoter operably linked with a base editing enzyme coding sequence, wherein the base editing enzyme coding sequence is operably linked with a 3′UTR/miR sequence and a polyadenylation signal. In various embodiments, the base editing system targets the erythroid bcl11a enhancer and the BCL11A binding site of the HBG promoter, each of which contributes to causing γ-globin activation or re-activation. As disclosed herein, the base editing system can be self-inactivating, in that cleavage of donor vector by transposition results in degradation of non-integrated donor vector nucleic acids. In various embodiments, a miR sequence can be a sequence that suppresses Cas9 expression in a producer cell during HDAd35 donor vector production (see, e.g., Saydaminova et al., Mol. Ther. Meth. Clin. Dev. 1: 14057, 2015; Li et al., Mol. Ther. Meth. Clin. Dev. 9: 390-401, 2018).

In various embodiments, an Ad35 system of the present disclosure further includes an Ad35 support vector, where the support vector includes, optionally from 5′ to 3′, (a) a recombines gene including an EF1α promoter operably linked with a FLPe recombinase coding sequence, and (b) a transposase gene including a PGK promoter operably linked with an SB100x transposase coding sequence.

In various embodiments an Ad35 payload is present in an Ad35 donor vector genome. In various embodiments an Ad35 payload present in an Ad35 donor vector genome is flanked by Ad35 ITRs. In various embodiments, an Ad35 donor vector genome is present in an Ad35 donor vector. In various embodiments, the donor vector is an Ad35++ vector.

In various embodiments a support genome includes Ad35 ITRs. In various embodiments, a support genome is present in an Ad35 vector. In various embodiments, the support vector is an Ad35++ vector.

In various embodiments, an Ad35 donor vector is a helper dependent donor vector (HDAd35). In certain such embodiments, systems of the present disclosure can include an HDAd35 donor vector or genome, and Ad35 helper vector or genome, and in various embodiments can further include an Ad35 support vector.

(v) In various embodiments, an Ad35 payload includes an integration element flanked by transposase inverted repeats for transposition by SB100x, and the transposase inverted repeats are flanked by frt direct repeats for recombination by an FLP recombinase such as FLPe. In various embodiments, the integration element includes, optionally from 5′ to 3′, (a) a β-globin mini-LCR, (b) a gene including a β-globin promoter operably linked with a rhesus γ-globin coding sequence, which γ-globin coding sequence is operably linked with a 3′UTR (e.g., a γ-globin 3′UTR), where the β-globin mini-LCR is also operably linked with the γ-globin coding sequence (c) a cHS4 insulator sequence, and (d) a gene including a PGK promoter operably linked with an MGMT^{P140K} coding sequence, where the MGMT^{P140K} coding sequence is operably linked with a polyadenylation signal, optionally where any of (a)-(d) can be encoded in a 5′ to 3′ orientation on either of the two strands of an Ad35 payload.

In various embodiments, an Ad35 payload further includes, outside of the integration element and outside of the recombinase sites, a nucleic acid sequence encoding a base editing system. In certain particular embodiments, the nucleic acid sequence encoding a base editing system includes, optionally from 5′ to 3′, (a) a gRNA gene including a U6 promoter operably linked with a gRNA-encoding sequence, where the gRNA targets an HBG promoter, and (b) a base editing enzyme gene including an EF1α promoter operably linked with a base editing enzyme coding sequence, wherein the base editing enzyme coding sequence is operably linked with a 3′UTR/miR sequence and a polyadenylation signal. In various embodiments, the base editing system targets the BCL11A binding site of the HBG promoter, which can result in γ-globin activation or re-activation. As disclosed herein, the base editing system can be self-inactivating, in that cleavage of donor vector by transposition results in degradation of non-integrated donor vector nucleic acids. In various embodiments, a miR sequence can be a sequence that suppresses Cas9 expression in a producer cell during HDAd35 donor vector production (see, e.g., Saydaminova et al., Mol. Ther. Meth. Clin. Dev. 1: 14057, 2015; Li et al., Mol. Ther. Meth. Clin. Dev. 9: 390-401, 2018).

In various embodiments, an Ad35 system of the present disclosure further includes an Ad35 support vector, where the support vector includes, optionally from 5′ to 3′, (a) a recombines gene including an EF1α promoter operably linked with a FLPe recombinase coding sequence, and (b) a transposase gene including a PGK promoter operably linked with an SB100x transposase coding sequence.

In various embodiments an Ad35 payload is present in an Ad35 donor vector genome. In various embodiments an Ad35 payload present in an Ad35 donor vector genome is flanked by Ad35 ITRs. In various embodiments, an Ad35 donor vector genome is present in an Ad35 donor vector. In various embodiments, the donor vector is an Ad35++ vector.

In various embodiments a support genome includes Ad35 ITRs. In various embodiments, a support genome is present in an Ad35 vector. In various embodiments, the support vector is an Ad35++ vector.

In various embodiments, an Ad35 donor vector is a helper dependent donor vector (HDAd35). In certain such embodiments, systems of the present disclosure can include an HDAd35 donor vector or genome, and Ad35 helper vector or genome, and in various embodiments can further include an Ad35 support vector.

### I(C)(ii). Payload Regulatory Sequences

### I(C)(ii)(a). Promoter Regulatory Sequences

A promoter can be a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the enzyme and other related protein factors that attach to it and the rate of RNA synthesis. The RNA is processed to produce messenger RNA (mRNA) which serves as a template for translation of the RNA sequence into the amino acid sequence of the encoded polypeptide. The 5′ non-translated leader sequence is a region of the mRNA upstream of the coding region that may play a role in initiation and translation of the mRNA. The 3′ transcription termination/polyadenylation signal is a non-translated region downstream of the coding region that functions in the plant cell to cause termination of the RNA synthesis and the addition of polyadenylate nucleotides to the 3′ end.

Promoters can include general promoters, tissue-specific promoters, cell-specific promoters, and/or promoters specific for the cytoplasm. Promoters may include strong promoters, weak promoters, constitutive expression promoters, and/or inducible (conditional) promoters. Inducible promoters direct or control expression in response to certain conditions, signals, or cellular events. For example, the promoter may be an inducible promoter that requires a particular ligand, small molecule, transcription factor, hormone, or hormone protein in order to effect transcription from the promoter. Particular examples of promoters include the AFP (α-fetoprotein) promoter, amylase 1C promoter, aquaporin-5 (AP5) promoter, αl-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, COX-2 promoter, CXCR4 promoter, desmin promoter, E2F-1 promoter, human elongation factor Iα promoter (EFIα), CMV (cytomegalovirus viral) promoter, minCMV promoter, SV40 (simian virus 40) immediately early promoter, EGR1 promoter, eIF4A1 promoter, elastase-1 promoter, endoglin promoter, FerH promoter, FerL promoter, fibronectin promoter, Flt-1 promoter, GAPDH promoter, GFAP promoter, GPIIb promoter, GRP78 promoter, GRP94 promoter, HE4 promoter, hGR1/1 promoter, hNIS promoter, Hsp68 promoter, the Hsp68 minimal promoter (proHSP68), HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, Nphsl promoter, OG-2 promoter, PGK (Phospho Glycerate kinase) promoters, PGK-1 promoter, polymerase III (Pol III) promoter, PSA promoter, ROSA promoter, SP-B promoter, Survivn promoter, SYN1 promoter, SYT8 gene promoter, TRP1 promoter, Tyr promoter, ubiquitin B promoter, WASP promoter, and the Rous Sarcoma Virus (RSV) long-terminal repeat (LTR) promoter

Promoters may be obtained as native promoters or composite promoters. Native promoters, or minimal promoters, refer to promoters that include a nucleotide sequence from the 5′ region of a given gene. A native promoter includes a core promoter and its natural 5′UTR. In particular embodiments, the 5′UTR includes an intron. Composite promoters refer to promoters that are derived by combining promoter elements of different origins or by combining a distal enhancer with a minimal promoter of the same or different origin.

In particular embodiments, the SV40 promoter includes the sequence set forth in SEQ ID NO: 80. In particular embodiments, the dESV40 promoter (SV40 promoter with deletion of the enhancer region) includes the sequence set forth in SEQ ID NO: 81. In particular embodiments, the human telomerase catalytic subunit (hTERT) promoter includes the sequence set forth in SEQ ID NO: 82. In particular embodiments, the RSV promoter derived from the Schmidt-Ruppin A strain includes the sequence set forth in SEQ ID NO: 83. In particular embodiments, the hNIS promoter includes the sequence set forth in SEQ ID NO: 84. In particular embodiments, the human glucocorticoid receptor 1A (hGR 1/Ap/e) promoter includes the sequence set forth in SEQ ID NO: 85.

In particular embodiments, promoters include wild type promoter sequences and sequences with optional changes (including insertions, point mutations or deletions) at certain positions relative to the wild-type promoter. In particular embodiments, promoters vary from naturally occurring promoters by having 1 change per 20 nucleotide stretch, 2 changes per 20 nucleotide stretch, 3 changes per 20 nucleotide stretch, 4 changes per 20 nucleotide stretch, or 5 changes per 20 nucleotide stretch. In particular embodiments, the natural sequence will be altered in 1, 2, 3, 4, 5, 6, 7, 8, 9, or 10 bases. The promoter may vary in length, including from 50 nucleotides of LTR sequence to 100, 200, 250 or 350 nucleotides of LTR sequence, with or without other viral sequence.

Some promoters are specific to a tissue or cell and some promoters are non-specific to a tissue or cell. Each gene in mammalian cells has its own promoter and some promoters can only be activated in certain cell types. A non-specific promoter, or ubiquitous promoter, aids in initiation of transcription of a gene or nucleotide sequence that is operably linked to the promoter sequence in a wide range of cells, tissues and cell cycles. In particular embodiments, the promoter is a non-specific promoter. In particular embodiments, a non-specific promoter includes CMV promoter, RSV promoter, SV40 promoter, mammalian elongation factor 1a (EF1a) promoter, β-act promoter, EGR1 promoter, eIF4A1 promoter, FerH promoter, FerL promoter, GAPDH promoter, GRP78 promoter, GRP94 promoter, HSP70 promoter, β-Kin promoter, PGK-1 promoter, ROSA promoter, and/or ubiquitin B promoter.

A specific promoter aids in cell specific expression of a nucleotide sequence that is operably linked to the promoter sequence. In particular embodiments, a specific promoter is active in a B cells, monocytic cells, leukocytes, macrophages, pancreatic acinar cells, endothelial cells, astrocytes, and/or any other cell type or cell cycle. In particular embodiments, the promoter is a specific promoter. In particular embodiments, an SYT8 gene promoter regulates gene expression in human islets (Xu, et al., Nat Struct Mol Biol., 2011, 18: 372-378). In particular embodiments, kallikrein promoter regulates gene expression in ductal cell specific salivary glands. In particular embodiments, the amylase 1C promoter regulates gene expression in acinar cells. In particular embodiments, the aquaporin-5 (AP5) promoter regulates gene expression in acinar cells (Zheng and Baum, Methods Mol Biol., 434: 205-219, 2008). In particular embodiments, the B29 promoter regulates gene expression in B cells. In particular embodiments, the CD14 promoter regulates gene expression in monocytic cells. In particular embodiments, the CD43 promoter regulates gene expression in leukocytes and platelets. In particular embodiments, the CD45 promoter regulates gene expression in hematopoietic cells. In particular embodiments, the CD68 promoter regulates gene expression in macrophages. In particular embodiments, the desmin promoter regulates gene expression in muscle cells. In particular embodiments, the elastase-1 promoter regulates gene expression in pancreatic acinar cells. In particular embodiments, the endoglin promoter regulates gene expression in endothelial cells. In particular embodiments, the fibronectin promoter regulates gene expression in differentiating cells or healing tissue. In particular embodiments, the Flt-1 promoter regulates gene expression in endothelial cells. In particular embodiments, the GFAP promoter regulates gene expression in astrocytes. In particular embodiments, the GPllb promoter regulates gene expression in megakaryocytes. In particular embodiments, the ICAM-2 promoter regulates gene expression in endothelial cells. In particular embodiments, the Mb promoter regulates gene expression in muscle. In particular embodiments, the Nphsl promoter regulates gene expression in podocytes. In particular embodiments, the OG-2 promoter regulates gene expression in osteoblasts, odontoblasts. In particular embodiments, the SP-B promoter regulates gene expression in lung cells. In particular embodiments, the SYN1 promoter regulates gene expression in neurons. In particular embodiments, the WASP promoter regulates gene expression in hematopoietic cells.

In particular embodiments, the promoter is a tumor-specific promoter. In particular embodiments, the AFP promoter regulates gene expression in hepatocellular carcinoma. In particular embodiments, the CCKAR promoter regulates gene expression in pancreatic cancer. In particular embodiments, the CEA promoter regulates gene expression in epithelial cancers. In particular embodiments, the c-erbB2 promoter regulates gene expression in breast and pancreas cancer. In particular embodiments, the COX-2 promoter regulates gene expression in tumors. In particular embodiments, the CXCR4 promoter regulates gene expression in tumors. In particular embodiments, the E2F-1 promoter regulates gene expression in tumors. In particular embodiments, the HE4 promoter regulates gene expression in tumors. In particular embodiments, the LP promoter regulates gene expression in tumors. In particular embodiments, the MUC1 promoter regulates gene expression in carcinoma cells. In particular embodiments, the PSA promoter regulates gene expression in prostate and prostate cancers. In particular embodiments, the Survivn promoter regulates gene expression in tumors. In particular embodiments, the TRP1 promoter regulates gene expression in melanocytes and melanoma. In particular embodiments, the Tyr promoter regulates gene expression in melanocytes and melanoma.

### I(C)(ii)(b). LCR Regulatory Sequences

Locus control regions are operationally defined by their ability to enhance the expression of linked genes to physiological levels in a tissue-specific and copy number-dependent manner at ectopic chromatin sites. Li et al., Blood, 2002, 100(9): 3077-3086.

The β-globin LCR is exemplary of at least some LCRs in at least several respects. For example, like many other LCRs, the β-globin LCR enhances expression (e.g., increased transcription, increased translation, and/or increased cell or tissue specificity) of operably linked genes or transgenes and includes DNAse hypersensitive (HS) regions understood by those of skill in the art to mediate the expression effects of the LCR. In addition, like many other LCRs, the β-globin LCR can be utilized in whole or in part, e.g., in that it can be utilized in nucleic acids that include a β-globin LCR sequence that includes all of the β-globin LCR HS regions (HSI-HS5) or includes a subset of the β-globin LCR HS regions (e.g., HSI-HS4).

An exemplary nucleic acid sequence for the Homo sapiens β-globin region on chromosome 11 is provided at GenBank Accession Number NG_000007. A β-globin long LCR can, in some instances, be or include a sequence located 6 to 22 kb 5′ to the first (embryonic) globin gene in the locus. A β-globin long LCR can include 5 DNAse I hypersensitive sites, 5′HSs 1 to 5. Li et al., Blood, 2002, 100(9): 3077-3086. NG_000007 provides the location of the restriction sites that delineate the DNAse I hypersensitive sites HSI, HS2, HS3, and HS4 within the Locus Control Region (e.g., the SnaBI and BstXI restriction sites of HS2, the HindIII and BamHI restriction sites of HS3, and the BamHI and BanII restriction sites of HS4), and is incorporated herein by reference in its entirety and particularly with respect to hyper sensitive site positions. The sequence and position of HSI is described, for example, by Pasceri et al., Ann NY Acad. Sci. 850:377-381, 1998; Pasceri et al., Blood. 92:653-663, 1998; and Milot et al., Cell. 87:105-114, 1996. In particular embodiments, the HS2 region extends from position 16,671 to 17,058 of the Locus Control Region. The SnaBI and BstXI restriction sites of HS2 are located at positions 17,093 and 16,240, respectively. The HS3 region extends from position 12,459 to 13,097 of the Locus Control Region. The BamHI and HindIII restriction sites of HS3 are located at positions 12,065 and 13,360, respectively. The HS4 region extends from position 9,048 to 9,713 of the Locus Control Region. The BamHI and BanII restriction sites of HS4 are located at positions 8,496 and 9,576 respectively.

Particular embodiments disclosed herein utilize mini-portions of the β-globin LCR. Mini-portions include less than all 5 HS regions, such as HS1, HS2, HS3, HS4, and/or HS5, so long as the LCR does not include all 5 segments of the β-globin LCR. The 4.3 kb HS1-HS4 LCR utilized in Example 1 of the disclosure provides one example of a mini-LCR. Other mini-LCR can include, for example, HS1, HS2, and HS3; HS2, HS3, and HS4; HS3, HS4, and HS5; HS1, HS3, and HS5; HS1, HS2, and HS5; and HS1, HS4, and HS5. For additional examples of mini-LCR, see Sadelain et al., Proc. Nat. Acad. Sci. (USA) 92: 6728-6732, 1995; and Lebouich et al., EMBO J. 13: 3065-3076, 1994. Particular embodiments can utilize a mini-β-globin LCR in combination with a β-globin promoter. In particular embodiments, this combination yields a 5.9 kb LCR-promoter combination. In relation to LCR, “mini” and “micro” are used interchangeably herein.

Particular embodiments disclosed herein utilize long portions of the locus control region (LCR). A long β-globin LCR can include HS1, HS2, HS3, HS4, and HS5. In particular embodiments, a long LCR includes an 21.5 kb sequence including HS1, HS2, HS3, HS4, and HS5 of the β-globin LCR. A long β-globin LCR can be coupled with the β-globin promoter to drive high protein expression levels.

Particular embodiments can include as a long β-globin LCR positions 5292319-5270789 (21,531 bp) of human chromosome 11 (SEQ ID NO: 185) as enumerated in GRCh38. In various embodiments, a long LCR can have a total length equal to or greater than, 18 kb, 18.5 kb, 19 kb, 19.5 kb, 20 kb, 20.5 kb, 21 kb, 21.5 kb, or 21.531 kb. In various embodiments, a long LCR can have a total length equal to or greater than 70%, 75%, 80%, 85%, 90%, 91%, 92%, 93%, 94%, 95%, 96%, 97%, 98%, or 99% of the length of SEQ ID NO: 185. In various embodiments, a long LCR can include at least 18 kb, 18.5 kb, 19 kb, 19.5 kb, 20 kb, 20.5 kb, 21 kb, or 21.5 kb of SEQ ID NO: 185. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of SEQ ID NO: 185. In any of the various embodiments provided herein, a long LCR can include HS1, HS2, HS3, HS4, and HS5.

In various embodiments, an Ad35 vector system can include, e.g., a transposable transgene insert that includes positions 5228631-5227023 (1609 bp) of human chromosome 11 or 5228631-5227018 (1614 bp) (SEQ ID NO: 186) as enumerated in GRCh38 as a β-globin promoter. In various embodiments, a β-globin promoter can have a total length equal to or greater than, e.g., 1.0 kb, 1.1. kb, 1.2 kb, 1.3 kb, 1.4 kb, 1.5 kb, 1.6 kb, or 1.609 kb. In various embodiments, a β-globin promoter can include at least 1.0 kb, 1.1. kb, 1.2 kb, 1.3 kb, 1.4 kb, 1.5 kb, 1.6 kb, or 1.609 kb of SEQ ID NO: 186. In various embodiments, the transposable transgene insert can include positions 5228631-5227023 (1609 bp) of human chromosome 11. In various embodiments, a β-globin promoter can include a total length equal to or greater than, e.g., 100 bp, 200 bp, 300 bp, 400 bp, 500 bp, 1 kb, 1.5 kb, 2 kb, 2.5 kb, 3 kb, 4 kb, or 5 kb of a nucleic acid sequence upstream of, e.g., immediately upstream of the first coding nucleotide of, a gene whose expression is regulated by the β-globin LCR, including without limitation any of epsilon (HBE1), G-gamma (HBG2), A-gamma (HBG1), delta (HBD), and beta (HBB) globin genes and/or one or more genes present in the hemoglobin β locus (11:5,225,463-5,227,070, complement). In various embodiments, a β-globin promoter can include a total length equal to or greater than, e.g., 100 bp, 200 bp, 300 bp, 400 bp, 500 bp, 1 kb, 1.5 kb, 2 kb, 2.5 kb, 3 kb, 4 kb, or 5 kb of a nucleic acid sequence upstream, e.g., immediately upstream, of Chromosome 11 NC_000011.10 position 5227021. In various embodiments, a β-globin promoter can have a total length equal to or greater than 70%, 75%, 80%, 85%, 90%, 91%, 92%, 93%, 94%, 95%, 96%, 97%, 98%, or 99% of the length of SEQ ID NO: 186. In any of the various embodiments provided herein, a β-globin promoter can be or include a nucleic acid having a sequence having at least 70%, 75%, 80%, 85%, 90%, 91%, 92%, 93%, 94%, 95%, 96%, 97%, 98%, or 99% identity with a corresponding contiguous portion of SEQ ID NO: 186.

In various embodiments, a β-globin LCR, such as a long β-globin LCR, causes expression of an operably linked coding sequence in erythrocytes. In various embodiments, the operably linked coding sequence is also operably linked with a β-globin promoter as set forth herein or otherwise known in the art.

The immunoglobulin heavy chain locus B cell LCR is an exemplary LCR that enhances expression (e.g., increases transcription, increases translation, and/or increases cell or tissue specificity) of operably linked coding sequences. Expression of a coding sequence can be enhanced when operably linked to a immunoglobulin heavy chain locus B cell LCR that includes the complete immunoglobulin heavy chain locus B cell LCR sequence and/or that includes an expression-regulatory fragment thereof. The immunoglobulin heavy chain locus B cell LCR includes DNAse hypersensitive sites (HS) understood by those of skill in the art to mediate at least some of the expression-enhancing effects of the immunoglobulin heavy chain locus B cell LCR. The immunoglobulin heavy chain locus B cell LCR includes four DNase I-hypersensitive sites (HS1, HS2, HS3, and HS4) in the 3′Ca region of the immunoglobulin heavy chain (IgH) locus functions as an enhancer-locus control region (LCR). Accordingly, a immunoglobulin heavy chain locus B cell LCR can be a complete immunoglobulin heavy chain locus B cell LCR including all of HS1-HS4, or can be an expression-regulatory fragment thereof that includes a subset of the hypersensitive sites HS1-HS4. These HS sites map to 10-30 kb of the IgH C gene and can cause lymphoid cell-specific and developmentally regulated enhancer elements in transient transfection assays. It has been observed that this nucleic acid sequence can direct a similar pattern of expression when linked to c-myc genes in Burkitt Lymphoma and plasmacytoma cell lines. In Burkitt Lymphomas and plasmacytomas, control of c-myc by the B-cell LCR occurs because of characteristic chromosome translocations that cause c-myc genes to become juxtaposed with the IgH sequences, thereby resulting in aberrant c-myc transcription. Additional description of the B Cell LCR can be found, for example, in Madisen et al., Mol Cell Biol. 18(11):6281-92, 1998; Giannini et al, J. Immunol. 150:1772-1780, 1993; Madisen & Groudine, Genes Dev. 8:2212-2226, 1994; and Michaelson et al., Nucleic Acids Res. 23:975-981, 1995.

Expression constructs can additionally include features that enhance the stability of mRNA transcripts, for example, insulators, and/or polyA tails.

### I(C)(ii)(c). Micro RNA Site Regulatory Sequences

In various embodiments, a microRNA (or miRNA) control system can refer to a method or composition in which expression of a gene is regulated by the presence of microRNA sites (e.g., nucleic acid sequences with which a microRNA can interact). In various embodiments, the present disclosure includes an Ad35 donor vector that includes a payload in which a nucleic acid sequence encoding an expression product is operably linked to an miRNA target site such that expression of the expression product is controlled by presence, level, activity, and/or contact with a corresponding miRNA. In various embodiments, the miRNA site is a target site for an miRNA selected from any of miR423-5, miR423-5p, miR42-2, miR181c, miR125a, miR15a, miR187, and/or miR218. For the avoidance of doubt the present disclosure contemplates that a nucleic acid sequence operably linked with an miRNA site, e.g., as disclosed herein can be a nucleic acid sequence that encodes, e.g., any of one or more expression products provided herein.

In particular embodiments, a microRNA control system regulated expression of a gene such that the gene is expressed exclusively in target cells, such as HSPCs e.g., tumor infiltrating HSPCs. In some embodiments, a nucleic acid (e.g., a therapeutic gene) encoding a protein or nucleic acid of interest (e.g., an anti-cancer agent such as a CAR, TCR, antibody, and/or checkpoint inhibitor, e.g., an αPD-L1 antibody (e.g., an αPD-L1γ1 antibody) that is a checkpoint inhibitor) includes, is associated with, or is operably linked with a microRNA site, a plurality of same microRNA sites, or a plurality of distinct microRNA sites. While those of skill in the art will be familiar with means and techniques of associating a microRNA site with a nucleic acid or portion thereof having a sequence that encodes a gene of interest, certain non-limiting examples are provided herein. For example, a gene of interest (e.g., a sequence encoding an αPD-L1γ1 antibody) can be present in a nucleic acid such that expression of the gene of interest is regulated by the presence of one or more microRNA sites that suppress expression in cells that are not tumor-infiltrating leukocyte cells, but do not suppress expression in tumor-infiltrating leukocytes. In certain particular examples, a gene of interest (e.g., a sequence encoding an αPD-L1γ1 antibody) can be present in a nucleic acid such that expression of the gene of interest is regulated by the presence of one or more miR423-5p microRNA sites that suppress expression in cells that are not tumor-infiltrating leukocyte cells, but do not suppressed expression in tumor-infiltrating leukocytes. In various embodiments, a microRNA control system can include a nucleic acid that includes, or in which expression of a protein or nucleic acid of interest is regulated by, one or more microRNA sites, e.g., 1, 2, 3, 4, 5, 6, 7, 8, 9, 10, or more microRNA sites. In various embodiments, a microRNA control system can include a nucleic acid that includes, or in which expression of a protein or nucleic acid of interest is regulated by, one or more miR423-5p microRNA sites, e.g., 1, 2, 3, 4, 5, 6, 7, 8, 9, 10, or more miR423-5p microRNA sites. In some particular embodiments, a microRNA control system can include a nucleic acid that encodes αPD-L1γ1 antibody and includes, or in which expression of αPD-L1γ1 antibody is regulated by, one or more miR423-5p microRNA sites, e.g., 1, 2, 3, 4, 5, 6, 7, 8, 9, 10, or more miR423-5p microRNA sites, e.g., miR423-5p microRNA sites.

In various embodiments, a microRNA site can be a sequence that suppresses expression of an operably linked coding sequence in a producer cell during HDAd35 donor vector production, e.g., a coding sequence encoding a CRISPR enzyme, base editing enzyme, or gRNA (see, e.g., Saydaminova et al., Mol. Ther. Meth. Clin. Dev. 1: 14057, 2015; Li et al., Mol. Ther. Meth. Clin. Dev. 9: 390-401, 2018).

### I(C)(iii). Selection Sequences

In particular embodiments vectors include a selection element including a selection cassette. In particular embodiments, a selection cassette includes a promoter, a cDNA that adds or confers resistance to a selection agent, and a poly A sequence enabling stopping the transcription of this independent transcriptional element.

A selection cassette can encode one or more proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cell lines. In particular embodiments, a positive selection cassette includes resistance genes to neomycin, hygromycin, ampicillin, puromycin, phleomycin, zeomycin, blasticidin, viomycin. In particular embodiments, a positive selection cassette includes the DHFR (dihydrofolate reductase) gene providing resistance to methotrexate, the MGMT^{P140K} gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) and other genes for detoxification with respect to some drugs. In particular embodiments, the selection agent includes neomycin, hygromycin, puromycin, phleomycin, zeomycin, blasticidin, viomycin, ampicillin, O⁶BG/BCNU, methotrexate, tetracycline, aminopterin, hypoxanthine, thymidine kinase, DHFR, Gln synthetase, or ADA.

In particular embodiments, negative selection cassettes include a gene for transformation of a substrate present in the culture medium into a toxic substance for the cell that expresses the gene. These molecules include detoxification genes of diphtheria toxin (DTA) (Yagi et al., Anal Biochem. 214(1):77-86, 1993; Yanagawa et al., Transgenic Res. 8(3):215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium. and for all positive and negative selections, a poly A transcription termination sequence from different origins, the most classical being derived from SV40 poly A, or a eukaryotic gene poly A (bovine growth hormone, rabbit β-globin, etc.).

In particular embodiments, the selection cassette includes MGMT^{P140K} as described in Olszko et al. (Gene Therapy 22: 591-595, 2015). In particular elements, the selection agent includes O⁶BG/BCN U.

The drug resistant gene MGMT encoding human alkyl guanine transferase (hAGT) is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG but retain their ability to repair DNA damage (Maze et al., J. Pharmacol. Exp. Ther. 290: 1467-1474, 1999). MGMT^{P140K}-based drug resistant gene therapy has been shown to confer chemoprotection to mouse, canine, rhesus macaques, and human cells, specifically hematopoietic cells (Zielske et al., J. Clin. Invest. 112: 1561-1570, 2003; Pollok et al., Hum. Gene Ther. 14: 1703-1714, 2003; Gerull et al., Hum. Gene Ther. 18: 451-456, 2007; Neff et al., Blood 105: 997-1002, 2005; Larochelle et al., J. Clin. Invest. 119: 1952-1963, 2009; Sawai et al., Mol. Ther. 3: 78-87, 2001).

In particular embodiments, combination with an in vivo selection cassette will be a critical component for diseases without a selective advantage of gene-corrected cells. For example, in SCID and some other immunodeficiencies and FA, corrected cells have an advantage and only transducing the therapeutic gene into a “few” HSPCs is sufficient for therapeutic efficacy. For other diseases like hemoglobinopathies (i.e., sickle cell disease and thalassemia) in which cells do not demonstrate a competitive advantage, in vivo selection of the gene corrected cells, such as in combination with an in vivo selection cassette such as MGMT^{P140K}, will select for the few transduced HSPCs, allowing an increase in the gene corrected cells and in order to achieve therapeutic efficacy. This approach can also be applied to HIV by making HSPCs resistant to HIV in vivo rather than ex vivo genetic modification.

### I(C)(iv). Stuffer Sequences

In particular embodiments, the vector includes a stuffer sequence. In particular embodiments, the stuffer sequence may be added to render the genome at a size near that of wild-type length. Stuffer is a term generally recognized in the art intended to define functionally inert sequence intended to extend the length

The stuffer sequence is used to achieve efficient packaging and stability of the vector. In particular embodiments, the stuffer sequence is used to render the genome size between 70% and 110% of that of the wild type virus.

The stuffer sequences can be any DNA, preferably of mammalian origin. In a preferred embodiment of the invention, stuffer sequences are non-coding sequences of mammalian origin, for example intronic fragments.

The stuffer sequence, when used to keep the size of the vector a predetermined size, can be any non-coding coding sequence or sequence that allows the genome to remain stable in dividing or nondividing cells. These sequences can be derived from other viral genomes (e.g. Epstein bar virus) or organism (e.g. yeast). For example, these sequences could be a functional part of centromeres and/or telomeres.

### I(C)(v). Payload Integration and Support Vectors

Gene therapy often requires integration of a desired nucleic acid payload into the genome of a target cell. A variety of systems can be designed and/or used for integration of a payload into a host or target cell genome. Various such systems can include one or more of certain payload sequence features and support vectors and support genomes (support genomes).

One means of engineering adenoviral vectors that integrate a payload into a host cell genome has been to produce integrating viral hybrid vectors. Integrating viral hybrid vectors combine genetic elements of a vector that efficiently transduces target cells with genetic elements of a vector that stably integrates its vector payload. Integration elements of interest, e.g., for use in combination with adenoviral vectors, have included those of bacteriophage integrase PHiC31, retrotransposons, retrovirus (e.g., LTR-mediated or retrovirus integrate-mediated), zinc-finger nuclease, DNA-binding domain-retroviral integrase fusion proteins, AAV (e.g., AAV-ITR or AAV-Rep protein-mediated), and Sleeping Beauty (SB) transposase.

Ad35 vectors described herein can optionally include transposable elements including transposases and transposons. Transposases can include integrases from retrotransposons or of retroviral origin, as well as an enzyme that is a component of a functional nucleic acid-protein complex capable of transposition and which is mediating transposition. A transposition reaction includes a transposon and a transposase or an integrase enzyme. In particular embodiments, the efficiency of integration, the size of the DNA sequence that can be integrated, and the number of copies of a DNA sequence that can be integrated into a genome can be improved by using such transposable elements. Transposons include a short nucleic acid sequence with terminal repeat sequences upstream and downstream of a larger segment of DNA. Transposases bind the terminal repeat sequences and catalyze the movement of the transposon to another portion of the genome.

A number of transposases have been described in the art that facilitate insertion of nucleic acids into the genome of vertebrates, including humans. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himarl, Passport, Minos, Ac/Ds, PIF, Harbinger, Harbinger3-DR, HSmar1, and spinON.

The PiggyBac (PB) transposase is a compact functional transposase protein that is described in, for example, Fraser et al., Insect Mol. Biol., 1996, 5, 141-51; Mitra et al., EMBO J., 2008, 27, 1097-1109; Ding et al., Cell, 2005, 122, 473-83; and U.S. Pat. Nos. 6,218,185; 6,551,825; 6,962,810; 7,105,343; and 7,932,088. Hyperactive piggyBac transposases are described in U.S. Pat. No. 10,131,885.

In particular embodiments, PB transposase has the sequence as set forth in SEQ ID NO: 291 (GenBank ABS12111.1).

In particular embodiments, a Frog Prince transposase has the sequence as set forth in SEQ ID NO: 292 (GenBank: AAP49009.1). See also US2005/0241007.

In particular embodiments, a TcBuster transposase has the sequence as set forth in SEQ ID NO: 293 (GenBank: ABF20545.1).

In particular embodiments, a Tol2 transposase has the sequence set forth in SEQ ID NO: 294 (GenBank: BAA87039.1).

Additional information on DNA transposons can be found, for instance, in Muñoz-López & Garcia Pérez, Curr Genomics, 11(2):115-128, 2010.

Sleeping Beauty is described in Ivics et al. Cell 91, 501-510, 1997; Izsvak et al., J. Mol. Biol., 302(1):93-102, 2000; Geurts et al., Molecular Therapy, 8(1): 108-117, 2003; Mates et al. Nature Genetics 41:753-761, 2009; and U.S. Pat. Nos. 6,489,458; 7,148,203; and 7,160,682; US Publication Nos. 2011/117072; 2004/077572; and 2006/252140. In certain embodiments, the Sleeping Beauty transposase enzyme has the sequence SEQ ID NO: 73. In particular embodiments, the Hyperactive Sleeping Beauty (SB100x) transposase enzyme has the sequence SEQ ID NO: 74.

Systematic mutagenesis studies have been undertaken to increase the activity of the SB transposase. For example, Yant et al., undertook the systematic exchange of the N-terminal 95 AA of the SB transposase for alanine (Mol. Cell Biol. 24: 9239-9247, 2004). Ten of these substitutions caused hyperactivity between 200-400% as compared to SB10 as a reference. SB16, described in Baus et al. (Mol. Therapy 12: 1148-1156, 2005) was reported to have a 16-fold activity increase as compared to SB10. Additional hyperactive SB variants are described in Zayed et al. (Molecular Therapy 9(2):292-304, 2004) and U.S. Pat. No. 9,840,696.

SB transposons need to circularize in order to transpose (Yant et al., Nature Biotechnology, 20: 999-1005, 2002). Furthermore, there is an inverse linear relationship, for transposons between 1.9 and 7.2 kb, between the length of the transposon and transposition frequency. In other words, SB transposase mediate the delivery of larger transposons less efficiently compared to smaller transposons (Geurts et al., Mol Ther., 8(1):108-17, 2003).

SB transposases transpose nucleic acid transposon payloads that are positioned between SB ITRs. Various SB ITRs are known in the art. In some embodiments, an SB ITR is a 230 bp sequence including imperfect direct repeats of 32 bp in length that serve as recognition signals for the transposase. Engineered SB ITRs are known in the art, including SB ITRs known as pT, pT2, pT3, pT2B, and pT4. In some embodiments, pT4 ITRs are used, e.g., to flank a transposon payload of the present disclosure, e.g., for transposition by an SB100x transposase.

In particular embodiments, the sequence encoding the IR(inverted repeat)/DR(direct repeat) and chromosomal sequence of Sleeping Beauty includes SEQ ID NO: 4. In particular embodiments, the sequence encoding the IR/DR and chromosomal sequence of Sleeping Beauty includes SEQ ID NO: 5. In particular embodiments, the IR/DR encoding sequence of Sleeping Beauty includes SEQ ID NO: 295. In particular embodiments, the sequence encoding the IR/DR and chromosomal sequence of Sleeping Beauty includes SEQ ID NO: 296. In particular embodiments, the sequence encoding the IR/DR and chromosomal sequence of Sleeping Beauty includes SEQ ID NO: 297. In particular embodiments, the sequence encoding the IR/DR of Sleeping Beauty includes SEQ ID NO: 298. In particular embodiments, the sequence encoding the IR/DR and chromosomal sequence of Sleeping Beauty includes SEQ ID NO: 299. In particular embodiments, the sequence encoding the IR/DR of Sleeping Beauty includes SEQ ID NO: 300.

In various embodiments, an Ad35 donor vector or genome includes a payload that includes SB100x transposon inverted repeats that flank an integration element that includes at least one coding sequence that encodes a β-globin expression product or a γ-globin expression product.

In various embodiments, an adenoviral transposition system includes an Ad35 donor vector or genome that includes an integration element flanked by transposon inverted repeats, and can further include an adenoviral support vector or support genome. In various embodiments, a support vector includes (i) the adenoviral capsid; and (ii) an adenoviral support genome including a nucleic acid sequence encoding a transposase that corresponds to the inverted repeats that flank the integration element. Accordingly, in various embodiments, at least one function of a support vector or support genome can be to encode, express, and/or deliver to a target cell a transposase for transposition of an integration element present in a donor vector administered to the target cell. For instance, in some embodiments, an Ad35 donor vector or genome includes SB100x transposon inverted repeats that flank an integration element that includes at least one coding sequence that encodes a β-globin expression product or a γ-globin expression product, and a support vector or support genome includes a coding sequence that encodes SB100x transposase. In certain embodiments, an integration element is flanked by recombinase direct repeats, e.g., where the integration element is flanked by transposon inverted repeats and the transposon inverted repeats are flanked by recombinase direct repeats. In certain such embodiments, at least one function of a support vector or support genome can be to encode, express, and/or delivery to a target cell a recombinase for recombination of recombinase sites present in a donor vector administered to the target cell. In various embodiments, a support vector or support genome can encode, express, and/or delivery to a target cell a recombinase for recombination of recombinase sites present in a donor vector administered to the target cell and also encode, express, and/or deliver to a target cell a transposase for transposition of an integration element present in a donor vector administered to the target cell.

Particular embodiments disclosed herein also use site-specific recombinase systems. In these embodiments, in addition to at least one therapeutic gene, the transposon including transposase-recognized inverted repeats also includes at least one recombinase-recognized site. Thus, in particular embodiments, The present disclosure also provides methods of integrating a therapeutic gene into the genome including administering: (a) a transposon including the therapeutic gene, wherein the therapeutic gene is flanked by (i) an inverted repeat sequence recognized by a transposase and (ii) a recombinase-recognized site; and b) a transposase and recombinase that serve to excise the therapeutic gene from a plasmid, episome, or transgene and integrate the therapeutic gene into the genome. In some embodiments, the protein(s) of (b) are administered as a nucleic acid encoding the protein(s). In some embodiments, the transposon and the nucleic acids encoding the protein(s) of (b) are present on separate vectors. In some embodiments, the transposon and nucleic acid encoding the protein(s) of (b) are present on the same vector. When present on the same vector, the portion of the vector encoding the protein(s) of (b) are located outside the portion carrying the transposon of (a). In other words, the transposase and/or recombinase encoding region is located external to the region flanked by the inverted repeats and/or recombinase-recognition site. In the aforementioned methods, the transposase protein recognizes the inverted repeats that flank an inserted nucleic acid, such as a nucleic acid that is to be inserted into a target cell genome. The use of recombinases and recombinase-recognized sites can increase the size of a transposon that can be integrated into a genome further.

Examples of recombinase systems include the Flp/Frt system, the Cre/loxP system, the Dre/rox system, the Vika/vox system, and the PhiC31 system.

The Flp/Frt DNA recombinase system was isolated from Saccharomyces cerevisiae. The Flp/Frt system includes the recombinase Flp (flippase) that catalyzes DNA-recombination on its Frt recognition sites. In particular embodiments, Flp (flippase) includes the sequence SEQ ID NO: 75 and the FRT recognition site includes SEQ ID NO: 76.

Variants of the Flp protein include SEQ ID NO: 77 (GenBank: ABD57356.1) and SEQ ID NO: 78 (GenBank: ANW61888.1).

The Cre/loxP system is described in, for example, EP 0220000961. Cre is a site-specific DNA recombinase isolated from bacteriophage P1. In particular embodiments, Cre includes the sequence SEQ ID NO: 79.

The recognition site of the Cre protein is a nucleotide sequence of 34 base pairs, the loxP site (SEQ ID NO: 80). Cre recombines the 34 bp loxP DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine. Variants of the lox recognition site that can also be used include: 1ox2272 (SEQ ID NO: 81); lox511 (SEQ ID NO: 82); 1ox66 (SEQ ID NO: 83); lox71 (SEQ ID NO: 84); loxM2 (SEQ ID NO: 85); and 1ox5171 (SEQ ID NO: 86).

The VCre/VIoxP recombinase system was isolated from Vibrio plasmid p0908. In particular embodiments, the VCre recombinase of this system includes SEQ ID NO: 87 and the VloxP recognition site includes SEQ ID NO: 88.

The sCre/SloxP system is described in WO 2010/143606. The Dre/rox system is described in U.S. Pat. Nos. 7,422,889 and 7,915,037B2. It generally includes a Dre recombinase isolated from Enterobacteria phage D6 with the sequence SEQ ID NO: 89 and the rox recognition site (SEQ ID NO: 90).

The Vika/vox system is described in U.S. Pat. No. 10,253,332. Additionally, the PhiC31 recombinase recognizes the AttB/AttP binding sites.

The amount of vector nucleic acid including the transposon (including inverted repeats and/or recombinase recognition sites), and in many embodiments the amount of vector nucleic acid encoding the transposase and/or recombinase, are introduced into the cell is sufficient to provide for the desired excision and insertion of the transposon nucleic acid into the target cell genome. As such, the amount of vector nucleic acid introduced should provide for a sufficient amount of transposase activity and/or recombinase activity and a sufficient copy number of the transposon that is desired to be inserted into the target cell genome. Particular embodiments include a 1:1; 1:2; or 1:3 ratio of transposon to transposase/recombinase.

The subject methods result in stable integration of the nucleic acid into the target cell genome. By stable integration is meant that the nucleic acid remains present in the target cell genome for more than a transient period of time and is passed on a part of the chromosomal genetic material to the progeny of the target cell.

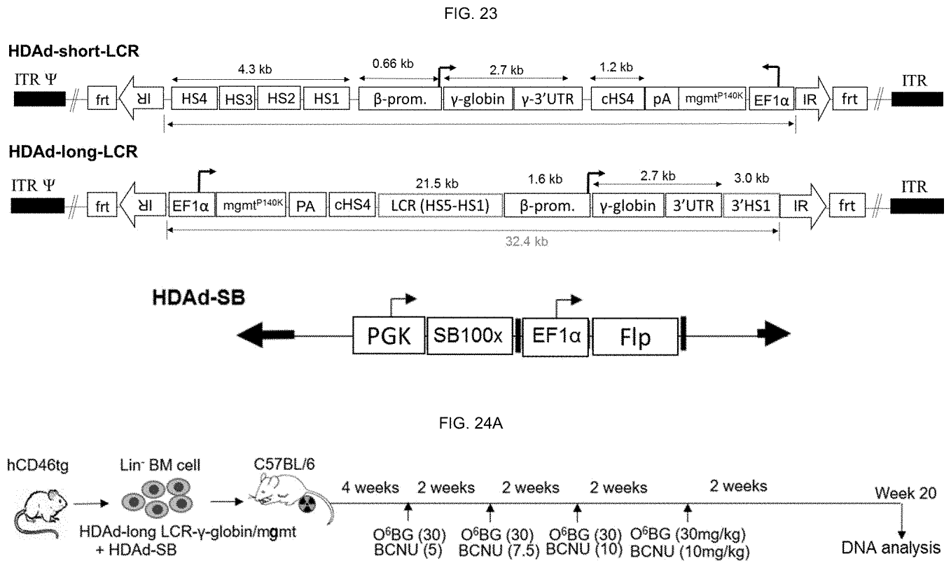

Example 2 of the current disclosure describes the surprising result that the hyperactive Sleeping Beauty transposase can be used to integrate a 32.4 kb transposon into the genome of HSPC. These embodiments include the use of SBX100 in combination with the Flp/Frt system as depicted in FIG. 23.

As indicated previously, particular embodiments utilize homology arms to facilitate targeted insertion of genetic constructs utilizing homology directed repair. Homology arms can be any length with sufficient homology to a genomic sequence at a cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. Homology arms are generally identical to the genomic sequence, for example, to the genomic region in which the double stranded break (DSB) occurs. However, as indicated, absolute identity is not required.

Particular embodiment can utilize homology arms with 25, 50, 100, or 200 nucleotides (nt), or more than 200 nt of sequence homology between a homology-directed repair template and a targeted genomic sequence (or any integral value between 10 and 200 nucleotides, or more). In particular embodiments, homology arms are 40-1000 nt in length. In particular embodiments, homology arms 500-2500 base pairs, 700-2000 base pairs, or 800-1800 base pairs. In particular embodiments, homology arms include at least 800 base pairs or at least 850 base pairs. The length of homology arms can also be symmetric or asymmetric.

Particular embodiment can utilize first and/or second homology arms each including at least 25, 50, 100, 200, 400, 600, 800, 1,000, 1,200, 1,400, 1,600, 1,800, 2,000, 2,500, or 3,000 nucleotides or more, having sequence identity or homology with a corresponding fragment of a target genome. In some embodiments, first and/or second homology arms each include a number of nucleotides having sequence identity or homology with a corresponding fragment of a target genome that has a lower bound of 25, 50, 100, 200, 400, 600, 800, 1,000, 1,200, 1,400, 1,600, or 1,800 nucleotides and an upper bound of 1,000, 1,200, 1,400, 1,600, 1,800, 2,000, 2,500, or 3,000 nucleotides. In some embodiments, first and/or second homology arms each include a number of nucleotides having sequence identity or homology with a corresponding fragment of a target genome that is between 40 and 1,000 nucleotides, between 500 and 2,500 nucleotides, between 700 and 2,000 nucleotides, or between 800 and 1800 nucleotides, or that has a length of at least 800 nucleotides or at least 850 nucleotides. First and second homology arms can have same, similar, or different lengths.

For additional information regarding homology arms, see Richardson et al., Nat Biotechnol. 34(3):339-44, 2016.

In particular embodiments, genetic constructs (e.g., genes leading to expression of a therapeutic product within a cell) are precisely inserted within genomic safe harbors. Genomic safe harbor sites are intragenic or extragenic regions of the genome that are able to accommodate the predictable expression of newly integrated DNA without adverse effects on the host cell. A useful safe harbor must permit sufficient transgene expression to yield desired levels of the encoded protein. A genomic safe harbor site also must not alter cellular functions. Methods for identifying genomic safe harbor sites are described in Sadelain et al., Nature Reviews 12:51-58, 2012; and Papapetrou et al., Nat Biotechnol. 29(1):73-8, 2011. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, to meet the criteria of a genomic safe harbor, chromatin sites must be >150 kb away from a known oncogene, >30 kb away from a known transcription start site; and have no overlap with coding mRNA. In particular embodiments, to meet the criteria of a genomic safe harbor, chromatin sites must be >200 kb away from a known oncogene, >40 kb away from a known transcription start site; and have no overlap with coding mRNA. In particular embodiments, to meet the criteria of a genomic safe harbor, chromatin sites must be >300 kb away from a known oncogene, >50 kb away from a known transcription start site; and have no overlap with coding mRNA. In particular embodiments, a genomic safe harbor meets the preceding criteria (>150 kb, >200 kb or >300 kb away from a known transcription start site; and have no overlap with coding mRNA >40 kb, or >50 kb away from a known transcription start site with no overlap with coding mRNA) and additionally is 100% homologous between an animal of a relevant animal model and the human genome to permit rapid clinical translation of relevant findings.

In particular embodiments, a genomic safe harbor meets criteria described herein and also demonstrates a 1:1 ratio of forward:reverse orientations of lentiviral integration further demonstrating the loci does not impact surrounding genetic material.

Particular genomic safe harbors sites include CCR5, HPRT, AAVS1, Rosa and albumin. See also, e.g., U.S. Pat. Nos. 7,951,925 and 8,110,379; U.S. Publication Nos. 2008/0159996; 2010/00218264; 2012/0017290; 2011/0265198; 2013/0137104; 2013/0122591; 2013/0177983 and 2013/0177960 for additional information and options for appropriate genomic safe harbor integration sites.

Various technologies known in the art can be used to direct integration of an integration element at specific genomic loci such as genomic safe harbors. For example AAV-mediated gene targeting, as well as homologous recombination enhanced by the introduction of DNA double-strand breaks using site-specific endonucleases (zinc-finger nucleases, meganucleases, transcription activator-like effector (TALE) nucleases), and CRISPR/Cas systems are all tools that can mediate targeted insertion of foreign DNA at predetermined genomic loci such as genomic safe harbors. Immunosuppression regimens are described, e.g., in U.S. Provisional Application No. 63/009,218, which is incorporated herein by reference in its entirety and in particular with respect to immunosuppression regimens.

In certain embodiments, integration of an integration element at specific genomic loci such as genomic safe harbors can include homology-directed integration using CRISPR enzyme-mediated cleavage of a target genome. CRISPR enzyme (e.g., Cas9) cleaves double stranded DNA at a site specified by a guide RNA (gRNA). The double strand break can be repaired by homology-directed repair (HDR) when a donor template (such as an Ad35 payload integration element including left and right homology arms) is present. In various such methods, an integration element is a “repair template” in that it includes left and right homology arms (e.g., of 500-3,000 bp) for insertion into a cleaved target genome. CRISPR-mediated gene insertion can be several orders of magnitude more efficient compared with spontaneous recombination of DNA template, demonstrating that CRISPR-mediated gene insertion can be an effective tool for genome editing. Exemplary methods of homology-directed integration of a nucleic acid sequence into a specified genomic locus are known in the art, e.g., in Richardson et al. (Nat Biotechnol. 34(3):339-44, 2016).

In various embodiments, an adenoviral donor vector including an integration element for insertion at a genomic safe harbor of a target cell genome can cause integration of a nucleic acid sequence having a length of up to 15 kb. In various embodiments, an integration element for integration into a target cell genome at a genomic safe harbor can have a length of at least 1 kb, 2 kb, 3 kb, 4 kb, 5 kb, 6 kb, 7 kb, 8 kb, 9 kb, 10 kb, 11 kb, 12 kb, 13 kb, 14 kb, or 15 kb, e.g., where the length has a lower bound of 1 kb, 2 kb, 3 kb, 4 kb, or 5 kb and an upper bound of 10 kb, 11 kb, 12 kb, 13 kb, 14 kb, or 15 kb.

### II. TARGET CELL POPULATIONS

In various embodiments, Ad35 donor vectors and genomes of the present disclosure can transduce target cells of any of a variety of types, including without limitation HSCs, T cells, B cells, and tumor cells disclosed herein.

### II(A). HSCs

In particular embodiments, vector-targeted cell types include hematopoietic stem cells (HSCs). HSCs are targeted for in vivo genetic modification by binding CD46. As indicated, within the current disclosure, HSC are targeted for in vivo genetic modification by binding CD46. Vectors can include mutations disclosed herein to increase the specificity and/or strength of CD46 binding. HSC can also be identified by the following marker profiles: CD34+, Lin-CD34+CD38-CD45RA-CD9O+CD49f+(HSC1) and CD34+CD38-CD45RA-CD90− CD49f+ (HSC2). Human HSC1 can be identified by the following profiles: CD34+/CD38-/CD45RA-/CD90+ or CD34+/CD45RA-/CD90+ and mouse LT-HSC can be identified by Lin-Scal+ckit+CD150+CD48-F1t3-CD34− (where Lin represents the absence of expression of any marker of mature cells including CD3, Cd4, CD8, CD11 b, CD11 c, NK1.1, Gr1, and TER119). In particular embodiments, HSC are identified by a CD164+ profile. In particular embodiments, HSC are identified by a CD34+/CD164+ profile. For additional information regarding HSC marker profiles, see WO2017/218948.

### II(B). T Cells

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells, and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

In particular embodiments, CARs are genetically modified to be expressed in cytotoxic T-cells.

“Central memory” T-cells (or “TCM”) as used herein refers to an antigen experienced CTL that expresses CD62L or CCR7 and CD45RO on the surface thereof, and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA.

### II(C). B Cells

B cells are mediators of the humoral response and are responsible for production and release of antibodies specific to an antigen. Several types of B cells exist which can be characterized by key markers. In general, immature B cells express CD19, CD20, CD34, CD38, and CD45R, and as they mature the key expressed markers are CD19 and IgM.

### II(D). Tumors

In particular embodiments, vectors can target tumors. In particular embodiments, tumors are targeted by targeting receptors present on tumor cells and not on healthy cells. Tumors can be targeted for in vivo genetic modification by binding αv integrins. The αv integrins play an important role in angiogenesis. The αvβ3 and αvβ5 integrins are absent or expressed at low levels in normal endothelial cells but are induced in angiogenic vasculature of tumors (Brooks et al., Cell, 79: 1157-1164, 1994; Hammes et al., Nature Med, 2: 529-533, 1996). Aminopeptidase N/CD13 has recently been identified as an angiogenic receptor for the NGR motif (Burg et al., Cancer Res, 59:2869-74, 1999). Aminopeptidase N/CD13 is strongly expressed in the angiogenic blood vessels of cancer and in other angiogenic tissues.

In particular embodiments, vectors can target tumors by targeting cancer cell antigen epitopes. Cancer cell antigens are expressed by cancer cells or tumors.

In particular embodiments, cancer cell antigen epitopes are preferentially expressed by cancer cells. “Preferentially expressed” means that a cancer cell antigen is found at higher levels on cancer cells as compared to other cell types. In some instances, a cancer antigen epitope is only expressed by the targeted cancer cell type. In other instances, the cancer antigen is expressed on the targeted cancer cell type at least 25%, 35%, 45%, 55%, 65%, 75%, 85%, 95%, 96%, 97%, 98%, 99%, or 100% more than on non-targeted cells.

In particular embodiments, cancer cell antigens are significantly expressed on cancerous and healthy tissue. In particular embodiments, significantly expressed means that the use of a bi-specific antibody was stopped during development based on on-target/off-cancer toxicities. In particular embodiments, significantly expressed means the use of a bi-specific antibody requires warnings regarding potential negative side effects based on on-target/off-cancer toxicities. As one example, cetuximab is anti-EGFR antibody associated with a severe skin rash thought to be due to EGFR expression in the skin. Another example is Herceptin (trastuzumab), which is an anti-HER2 (ERBB2) antibody. Herceptin is associated with cardiotoxicity due to target expression in the heart. Moreover, targeting Her2 with a CAR-T cell was lethal in a patient due to on-target, off-cancer expression in the lung.

Table 12 provides examples of cancer antigens that are more likely to be co-expressed in particular cancer types.

TABLE 12

| Cancer Antigens Likely to be Co-Expressed | Cancer Type |
|---|---|
| CD19, CD20, CD22, ROR1, CD33, CD56, | Leukemia/Lymphoma |
| CLL-1, WT-1, CD123, PD-L1, EFGR | |
| B-cell maturation antigen (BCMA), PD-L1, | Multiple Myeloma |
| EFGR | |
| PSMA, WT1, Prostate Stem Cell antigen | Prostate Cancer |
| (PSCA), SV40 T, PD-L1, EFGR | |
| HER2, ERBB2, ROR1, PD-L1, EFGR, | Breast Cancer |
| MUC16, folate receptor (FOLR), CEA | |
| CD133, PD-L1, EFGR | Stem Cell Cancer |
| L1-CAM, MUC16, FOLR, Lewis Y, ROR1, | Ovarian Cancer |
| mesothelin, WT-1, PD-L1, EFGR, CD56 | |
| mesothelin, PD-L1, EFGR | Mesothelioma |
| carboxy-anhydrase-IX (CAIX); PD-L1, EFGR | Renal Cell Carcinoma |
| GD2, PD-L1, EFGR | Melanoma |
| mesothelin, CEA, CD24, ROR1, PD-L1, | Pancreatic Cancer |
| EFGR, MUC16 | |
| ROR1, PD-L1, EFGR, mesothelin, MUC16, | Lung Cancer |
| FOLR, CEA, CD56 | |
| mesothelin, PD-L1, EFGR | Cholangiocarcinoma |
| MUC16, PD-L1, EFGR, | Bladder Cancer |
| ROR1, glypican-2, CD56, disialoganglioside, | Neuroblastoma |
| PD-L1, EFGR, | |
| CEA, PD-L1, EFGR, | Colorectal Cancer |
| CD56, PD-L1, EFGR, | Merkel Cell Carcinoma |

In more particular examples, cancer cell antigens include: Mesothelin, MUC16, FOLR, PD-L1, ROR1, glypican-2 (GPC2), disialoganglioside (GD2), HER2, EGFR, EGFRvIII, CEA, CD56, CLL-1, CD19, CD20, CD123, CD30, CD33 (full length), CD33 (DeltaE2 variant), CD33 (with C-terminal truncation), BCMA, IGFR, MUC1, VEGFR, PSMA, PSCA, IL13Ra2, FAP, EpCAM, CD44, CD133, Tro-2, CD200, FLT3, GCC, and VVT1. As will be understood by one of ordinary skill in the art, targeted antigens can lack signal peptides.

CD56, also known as neural cell adhesion molecule 1 (NCAM1), is a type I membrane glycoprotein involved in cell-cell and cell-matrix adhesion. Its extracellular domain has five IgG-like domains at the N-terminus and two fibronectin type III domains in the membrane-proximal region.

Disialoganglioside GalAcbeta1-4(NeuAcalpha2-8NeuAcalpha2-3)Galbeta1-4Glcbeta1-1Cer (GD2) is expressed on various tumors, including neuroblastoma. The disialoganglioside antigen GD2 includes a backbone of oligosaccharides flanked by sialic acid and lipid residues. See, e.g., Cheresh (Surv. Synth. Pathol. Res. 4:97, 1987) and U.S. Pat. No. 5,653,977.

EGFR variant III (EGFRvIII), a tumor specific mutant of EGFR, is a product of genomic rearrangement which is often associated with wild-type EGFR gene amplification. EGFRvIII is formed by an in-frame deletion of exons 2-7, leading to deletion of 267 amino acids with a glycine substitution at the junction. The truncated receptor loses its ability to bind ligands but acquires constitutive kinase activity. Interestingly, EGFRvIII frequently co-expresses with full length wild-type EGFR in the same tumor cells. Moreover, EGFRvIII expressing cells exhibit increased proliferation, invasion, angiogenesis and resistance to apoptosis.

EGFRvIII is most often found in glioblastoma multiforme (GBM). It is estimated that 25-35% of GBM carries this truncated receptor. Moreover, its expression often reflects a more aggressive phenotype and poor prognosis. Besides GBM, expression of EGFRvIII has also been reported in other solid tumors such as non-small cell lung cancer, head and neck cancer, breast cancer, ovarian cancer and prostate cancer. In contrast, EGFRvIII is not expressed in healthy tissues.

In particular embodiments, a targeted cancer antigen epitope can have high expression by a targeted cancer cell or tumor or low expression by a targeted cancer cell or tumor. In particular embodiments, high and low expression can be determined using flow cytometry or fluorescence-activated cell-sorting (FACS). As is understood by one of ordinary skill in the art of flow cytometry, “hi”, “lo”, “+” and “−” refer to the intensity of a signal relative to negative or other populations. In particular embodiments, positive expression (+) means that the marker is detectable on a cell using flow cytometry. In particular embodiments, negative expression (−) means that the marker is not detectable using flow cytometry. In particular embodiments, “hi” means that the positive expression of a marker of interest is brighter as measured by fluorescence (using for example FACS) than other cells also positive for expression. In these embodiments, those of ordinary skill in the art recognize that brightness is based on a threshold of detection. Generally, one of skill in the art will analyze a negative control tube first, and set a gate (bitmap) around the population of interest by FSC and SSC and adjust the photomultiplier tube voltages and gains for fluorescence in the desired emission wavelengths, such that 97% of the cells appear unstained for the fluorescence marker with the negative control. Once these parameters are established, stained cells are analyzed, and fluorescence recorded as relative to the unstained fluorescent cell population. In particular embodiments, and representative of a typical FACS plot, hi implies to the farthest right (x line) or highest top line (upper right or left) while lo implies within the left lower quadrant or in the middle between the right and left quadrant (but shifted relative to the negative population). In particular embodiments, “hi” refers to greater than 20-fold of +, greater than 30-fold of +, greater than 40-fold of +, greater than 50-fold of +, greater than 60-fold of +, greater than 70-fold of +, greater than 80-fold of +, greater than 90-fold of +, greater than 100-fold of +, or more of an increase in detectable fluorescence relative to + cells. Conversely, “lo” can refer to a reciprocal population of those defined as “hi”.

### II(E). Other Targets

In addition to HSCs, T Cells, B Cells, and tumors (or cancer cells), vectors can target other antigens for bacteria and fungi.

Antigens targeting bacteria can be derived from, for example, anthrax, gram-negative bacilli, Chlamydia, diphtheria, Helicobacter pylori, Mycobacterium tuberculosis, pertussis toxin, pneumococcus, rickettsiae, Staphylococcus, Streptococcus and tetanus.

As particular examples of bacterial antigen markers, anthrax antigens include anthrax protective antigen; gram-negative bacilli antigens include lipopolysaccharides; diptheria antigens include diphtheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

Antigens targeting fungi can be derived from, for example, Candida, coccidiodes, Cryptococcus, Histoplasma, Leishmania, Plasmodium, protozoa, parasites, schistosomae, tinea, Toxoplasma, and Trypanosoma cruzi.

As particular examples of fungal antigens, coccidiodes antigens include spherule antigens; cryptococcal antigens include capsular polysaccharides; Histoplasma antigens include heat shock protein 60 (HSP60); Leishmania antigens include gp63 and lipophosphoglycan; Plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; tinea fungal antigens include Trichophyton; Toxoplasma antigens include SAG-1 and p30; and Trypanosoma cruzi antigens include the 75-77 kDa antigen and the 56 kDa antigen.

### III. DOSAGES, FORMULATIONS, AND ADMINISTRATION

A vector can be formulated such that it is pharmaceutically acceptable for administration to cells or animals, e.g., to humans. A vector may be administered in vitro, ex vivo, or in vivo. The Ad35 viral vector vectors described herein can be formulated for administration to a subject. Formulations include an Ad35 viral vector associated with a therapeutic gene (“active ingredient”) and one or more pharmaceutically acceptable carriers.

As disclosed herein, a vector can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations.

In addition, one skilled in the art may also contemplate additional delivery method may be via electroporation, sonophoresis, intraosseous injections methods or by using gene gun. Vectors may also be implanted into microchips, nano-chips or nanoparticles.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

A vector solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount can be an amount at which any toxic or detrimental effects of the composition are outweighed by therapeutically beneficial effects.

In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinositol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

In particular embodiments, the formulations include active ingredients of at least 0.1% w/v or w/w of the formulation; at least 1% w/v or w/w of formulation; at least 10% w/v or w/w of formulation; at least 20% w/v or w/w of formulation; at least 30% w/v or w/w of formulation; at least 40% w/v or w/w of formulation; at least 50% w/v or w/w of formulation; at least 60% w/v or w/w of formulation; at least 70% w/v or w/w of formulation; at least 80% w/v or w/w of formulation; at least 90% w/v or w/w of formulations; at least 95% w/v or w/w of formulation; or at least 99% w/v or w/w of formulation.

The actual dose and amount of an Ad35 viral vector and, in particular embodiments, of an Ad35 viral vector and mobilization factors, administered to a particular subject and concordant mobilization procedure and schedule can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts of Ad35 vector associated with a therapeutic gene can include doses ranging from, for example, 1×10⁷ to 50×10⁸ infection units (IU) or from 5×10⁷ to 20×10⁸ IU. In other examples, a dose can include 5×10⁷ IU, 6×10⁷ IU, 7×10⁷ IU, 8×10⁷ IU, 9×10⁷ IU, 1×10⁸ IU, 2×10⁸ IU, 3×10⁸ IU, 4×10⁸ IU, 5×10⁸ IU, 6×10⁸ IU, 7×10⁸ IU, 8×10⁸ IU, 9×10⁸ IU, 10×10⁸ IU, or more. In particular embodiments, a therapeutically effective amount of an Ad35 vector associated with a therapeutic gene includes 4×10⁸ IU. In particular embodiments, a therapeutically effective amount of an Ad35 vector associated with a therapeutic gene can be administered subcutaneously or intravenously. In particular embodiments, a therapeutically effective amount of an Ad35 vector associated with a therapeutic gene can be administered following administration with one or more mobilization factors.

In various embodiments of the present disclosure, an in vivo gene therapy includes administration of at least one viral gene therapy vector to a subject in combination with at least one immune suppression regimen. In an in vivo gene therapy including more than one vector species, such as a first vector that is a supported viral gene therapy vector in combination with a second vector that is a support vector, the first vector and the second vector can be administered in a single formulation or dosage form or in two separate formulations or dosage forms. In various embodiments, the first and second vectors can be administered at the same time or at different times, e.g., during the same one-hour period or during non-overlapping one-hour periods. In various embodiments, the first and second vectors can be administered at the same time or at different times, e.g., on the same day or on different days. In various embodiments, the first and second vectors can be administered at the same dosage or at different dosages, e.g., where the dosage is measured as the total number of viral particles or as a number of viral particles per kilogram of the subject. In various embodiments, the first and second vectors can be administered in a pre-defined ratio. In various embodiments, the ratio is in the range of 2:1 to 1:2, e.g., 1:1.

In various embodiments, a vector is administered to a subject in a single total dose on a single day. In various embodiments a vector is administered in two, three, four, or more unit doses that together constitute a total dose. In various embodiments, one unit dose of a vector is administered to a subject per day on each of one, two, three, four, or more consecutive days. In various embodiments, two unit doses of a vector are administered to a subject per day on each of one, two, three, four, or more consecutive days. Accordingly, in various embodiments, a daily dose can refer to the dose of vector received by a subject over the course of a day. In various embodiments, the term day refers to a twenty-four-hour period, such as a twenty-four-hour period from midnight of a first calendar date to midnight of the next calendar date.

In various embodiments, a unit dose, daily dose, or total dose of a vector, such as a viral gene therapy vector or support vector, or the total combined dose of a viral gene therapy vector and a support vector, can be at least 1E8, 5E8, 1E9, 5E9, 1E10, 5E10, 1E11, 5E11, 1E12, 5E12, 1E13, 5E13, 1E14, or 1E15 viral particles per kilogram (vp/kg). In various embodiments, a unit dose, daily dose, or total dose of a vector, such as a viral gene therapy vector or support vector, or the total combined dose of a viral gene therapy vector and a support vector, can fall within a range having a lower bound selected from 1E8, 5E8, 1E9, 5E9, 1E10, 5E10, 1E11, 5E11, 1E12, 5E12, 1E13, 5E13, 1E14, or 1E15 vp/kg and an upper bound selected from 1E8, 5E8, 1E9, 5E9, 1E10, 5E10, 1E11, 5E11, 1E12, 5E12, 1E13, 5E13, 1E14, or 1E15 vp/kg.

In various embodiments, a viral gene therapy vector is administered at a unit dose, daily dose, or total dose of at least 1E10, 5E10, 1E11, 5E11, 1E12, 5E12, 1E13, 5E13, 1E14, or 1E15 vp/kg and a support vector is administered at a unit dose, daily dose, or total dose of at least 1 E8, 5E8, 1E9, 5E9, 1E10, 5E10, 1E11, and 5E11 vp/kg, optionally where the unit dose, daily dose, or total dose of the viral gene therapy vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the support vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg.

In various embodiments, a support vector is administered at a unit dose, daily dose, or total dose of at least 1E10, 5E10, 1E11, 5E11, 1E12, 5E12, 1E13, 5E13, 1E14, or 1E15 vp/kg and a supported viral gene therapy vector is administered at a unit dose, daily dose, or total dose of at least 1E8, 5E8, 1E9, 5E9, 1E1 0, 5E10, 1E11, and 5E11 vp/kg, optionally where the unit dose, daily dose, or total dose of the support vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the supported viral gene therapy vector is within a range having a lower bound selected from 1 E8, 5E8, 1E9, 5E9, 1E10, and 5E10 vp/kg and an upper bound selected from 1E9, 5E9, 1E10, 5E10, 1E11, and 5E11 vp/kg. In various embodiments, a supported viral gene therapy vector and a support vector are administered in a pre-defined ratio. In various embodiments, the ratio is in the range of 2:1 to 1:2, e.g., 1:1.

### IV. APPLICATIONS

Methods and compositions provided herein are disclosed at least in part for use in in vivo gene therapy. However, for the avoidance of doubt, the present disclosure expressly includes the use of compositions and methods provided herein for ex-vivo engineering of cells and/or tissues, as well as in vitro uses including the engineering of cells and/or tissues for research purposes. Gene therapy includes use of a vector, genome, or system of the present disclosure in a method of introducing exogenous DNA into a host cell (such as a target cell) and/or a nucleic acid (such as a target nucleic acid, such as a target genome, e.g., the genome of a target cell). The present disclosure includes description and exemplification of compositions and methods relating to in vivo, in vitro, and ex vivo therapy that those of skill in the art will appreciate that various methods and compositions provided herein are generally applicable to introduction of a nucleic acid payload into a subject, e.g., a host or target cell. Because such compositions and methods are of general utility, e.g., in gene therapy, they are useful both as tools in gene therapy in general and in various particular conditions in particular, including those provided herein.

### IV(A). In Vivo Gene Therapy

Treatments using in vivo gene therapy, which includes the direct delivery of a viral vector to a patient, have been explored. In vivo gene therapy is an attractive approach because it may not require any genotoxic conditioning (or could require less genotoxic conditioning) nor ex vivo cell processing and thus could be adopted at many institutions worldwide, including those in developing countries, as the therapy could be administered through an injection, similar to what is already done worldwide for the delivery of vaccines. In various embodiments methods of in vivo gene therapy with adenoviral vectors of the present disclosure can include one or more steps of (i) target cell mobilization, (ii) immunosuppression, (iii) administration of a vector, genome, system or formulation provided herein, and/or (iv) selection of transduced cells and/or cells that have integrated an integration element of a payload of an adenoviral vector or genome.

The adenoviral vector formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts of one or more vectors, genomes, or systems of the present disclosure. Therapeutically effective amounts include those that provide effective amounts, prophylactic treatments, and/or therapeutic treatments.

IV(A)i. Mobilization of HSCs

Vectors described herein can be administered in coordination with mobilization factors. In certain embodiments, adenoviral vector formulations described herein can be administered in concert with HSPC mobilization. In particular embodiments, administration of adenoviral donor vector occurs concurrently with administration of one or more mobilization factors. In particular embodiments, administration of adenoviral donor vector follows administration of one or more mobilization factors. In particular embodiments, administration of adenoviral donor vector follows administration of a first one or more mobilization factors and occurs concurrently with administration of a second one or more mobilization factors. Agents for HSPC mobilization include, for example, granulocyte-colony stimulating factor (G-CSF), granulocyte macrophage colony stimulating factor (GM-CSF), AMD3100, SCF, S-CSF, a CXCR4 antagonist, a CXCR2 agonist, and Gro-Beta (GRO-β). In various embodiments, a CXCR4 antagonist is AMD3100 and/or a CXCR2 agonist is GRO-β.

G-CSF is a cytokine whose functions in HSPC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and formulations as disclosed herein, for example, Filgrastim (Neupogen®, Amgen Inc., Thousand Oaks, Calif.) and PEGylated Filgrastim (Pegfilgrastim, NEULASTA®, Amgen Inc., Thousand Oaks, Calif.).

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and formulations as disclosed herein, for example, Sargramostim (Leukine, Bayer Healthcare Pharmaceuticals, Seattle, Wash.) and molgramostim (Schering-Plough, Kenilworth, N.J.).

AMD3100 (MOZOBIL™, PLERIXAFOR™; Sanofi-Aventis, Paris, France), a synthetic organic molecule of the bicyclam class, is a chemokine receptor antagonist and reversibly inhibits SDF-1 binding to CXCR4, promoting HSPC mobilization. AMD3100 is approved to be used in combination with G-CSF for HSPC mobilization in patients with myeloma and lymphoma. The structure of AMD3100 is:

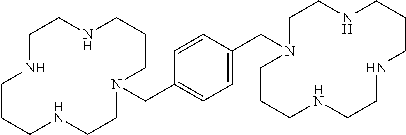 [SMILES: c1cc(CN2CCCNCCNCCCNCC2)ccc1CN1CCCNCCNCCCNCC1]

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and formulations as disclosed herein, for example, recombinant human SCF (Ancestim, STEMGEN®, Amgen Inc., Thousand Oaks, Calif.).

Chemotherapy used in intensive myelosuppressive treatments also mobilizes HSPCs to the peripheral blood as a result of compensatory neutrophil production following chemotherapy-induced aplasia. In particular embodiments, chemotherapeutic agents that can be used for mobilization of HSPCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israel); TG-0054 (Taigen Biotechnology, Taipei, Taiwan); CXCL12 neutralizer NOX-Al2 (NOXXON Pharma, Berlin, Germany) which binds to SDF-1, inhibiting its binding to CXCR4); Sphingosine-1-phosphate (SIP) agonists (e.g., SEW2871, Juarez et al. Blood 119: 707-716, 2012); vascular cell adhesion molecule-1 (VCAM) or very late antigen 4 (VLA-4) inhibitors (e.g., Natalizumab, a recombinant humanized monoclonal antibody against α4 subunit of VLA-4 (Zohren et al. Blood 111: 3893-3895, 2008); B105192, a small molecule inhibitor of VLA-4 (Ramirez et al. Blood 114: 1340-1343, 2009)); parathyroid hormone (Brunner et al. Exp Hematol. 36: 1157-1166, 2008); proteasome inhibitors (e.g., Bortezomib, Ghobadi et al. ASH Annual Meeting Abstracts. p. 583, 2012); Groβ, a member of CXC chemokine family which stimulates chemotaxis and activation of neutrophils by binding to the CXCR2 receptor (e.g., SB-251353, King et al. Blood 97: 1534-1542, 2001); stabilization of hypoxia inducible factor (HIF) (e.g., FG-4497, Forristal et al. ASH Annual Meeting Abstracts. p. 216, 2012); Firategrast, an α4β1 and α4β7 integrin inhibitor (α4β1/7) (Kim et al. Blood 128: 2457-2461, 2016); Vedolizumab, a humanized monoclonal antibody against the a4p7 integrin (Rosario et al. Clin Drug lnvestig 36: 913-923, 2016); and BOP (N-(benzenesulfonyl)-L-prolyl-L-O-(1-pyrrolidinylcarbonyl) tyrosine) which targets integrins α9β1/α4β1 (Cao et al. Nat Commun 7: 11007, 2016). Additional agents that can be used for HSPC mobilization are described in, for example, Richter R et al. Transfus Med Hemother 44:151-164, 2017, Bendall & Bradstock, Cytokine & Growth Factor Reviews 25: 355-367, 2014, WO 2003043651, WO 2005017160, WO 2011069336, U.S. Pat. Nos. 5,637,323, 7,288,521, 9,782,429, US 2002/0142462, and US 2010/02268.

In particular embodiments, a therapeutically effective amount of G-CSF includes 0.1 μg/kg to 100 μg/kg. In particular embodiments, a therapeutically effective amount of G-CSF includes 0.5 μg/kg to 50 μg/kg. In particular embodiments, a therapeutically effective amount of G-CSF includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before Ad35 delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to Ad35 administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before Ad35 delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to Ad35 administration. A dosing regimen for Sargramostim can include 200 μg/m², 210 μg/m², 220 μg/m², 230 μg/m², 240 μg/m², 250 μg/m², 260 μg/m², 270 μg/m², 280 μg/m², 290 μg/m², 300 μg/m², or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 μg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to Ad35 administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 μg/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 μg/kg 6 to 11 hours prior to Ad35 delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to Ad35 injection.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to Ad35 administration.

In particular embodiments, growth factors GM-CSF and G-CSF can be administered to mobilize HSPC in the bone marrow niches to the peripheral circulating blood to increase the fraction of HSPCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a S1P receptor 1 (S1PR1) modulator agent. US 20110044997 describes mobilization protocols utilizing a CXCR4 antagonist with a vascular endothelial growth factor receptor (VEGFR) agonist.

Ad35 viral vectors are an example of vectors that can be administered in concert with HSPC mobilization. In particular embodiments, administration of an Ad35 viral vector occurs concurrently with administration of one or more mobilization factors. In particular embodiments, administration of an Ad35 viral vector follows administration of one or more mobilization factors. In particular embodiments, administration of an Ad35 viral vector follows administration of a first one or more mobilization factors and occurs concurrently with administration of a second one or more mobilization factors.

In particular embodiments, an HSC enriching agent, such as a CD19 immunotoxin or 5-FU can be administered to enrich for HSPCs. CD19 immunotoxin can be used to deplete all CD19 lineage cells, which accounts for 30% of bone marrow cells. Depletion encourages exit from the bone marrow. By forcing HSPCs to proliferate (whether via CD19 immunotoxin of 5-FU, this stimulates their differentiation and exit from the bone marrow and increases transgene marking in peripheral blood cells.

Therapeutically effective amounts can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

IV(A)ii. Immunosuppression Regimens

Ad35 viral vectors can be administered concurrently with or following administration of one or more immunosuppression agents or immunosuppression regimens, which can include one or more steroids, IL-1 receptor antagonist, and/or an IL-6 receptor antagonist administration. These protocols can alleviate potential side effects of treatments.

IL-1 receptor antagonists are known and include ADC-1001 (Alligator Bioscience), FX-201 (Flexion Therapeutics), fusion proteins available from Bioasis Technologies, GQ-303 (Genequine Biotherapeutics GmbH), HL-2351 (Handok, Inc.), MBIL-1 RA (ProteoThera, Inc.), Anakinra (Pivor Pharmaceuticals), human immunoglobin G or Globulin S (GC Pharma). IL-6 receptor antagonists are also known in the art and include tocilizumab, BCD-089 (Biocad), HS-628 (Zhejiang Hisun Pharm), and APX-007 (Apexigen).

In various embodiments, an immune suppression regimen is administered to a subject that also receives at least one viral gene therapy vector, where the immune suppression regimen includes administration of at least one immune suppression agent to the subject on (i) one or more days prior to administration to the subject of a first dose of the viral gene therapy vector; (ii) on the same day as administration of a first dose of the viral gene therapy vector; (iii) on the same day as administration of one or more second or other subsequent doses of the viral gene therapy vector; and/or (iv) on any of one or more, or all, days intervening between administration to the subject of the first dose of the viral gene therapy vector and administration of any of one or more, or all, second or other subsequent doses of the viral gene therapy vector.

Immunosuppression regimens are further described, e.g., in U.S. Provisional Application No. 63/009,218, which is incorporated herein by reference in its entirety and in particular with respect to immunosuppression regimens.

IV(A)iii. Selection

In particular embodiments, methods of use include the treatment of conditions wherein corrected cells have a selective advantage over non-corrected cells. Ad35 viral vectors are an example of vectors that can be administered in concert with HSPC mobilization and prior to administration of selective agents that correspond with the in vivo selection cassette(s). Particular embodiments combine mobilization (e.g., a mobilization protocol described herein) with administration of an Ad35 vector described herein and BCNU or benzylguanine and temozolomide in the case of an Ad35 including a MGMT^{P140K} cassette and/or a CD33-targeting molecule in the case of the Ad35 vector including an anti-CD33 cassette.

In particular embodiments, in vivo Ad35-mediated gene delivery (with or without mobilization) can be combined with an in vivo selection marker. In particular embodiments, the in vivo selection marker can include MGMT^{P140K} as described in Olszko et al., Gene Therapy 22: 591-595, 2015.

The drug resistant gene MGMT encoding human alkyl guanine transferase (hAGT) is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze et al. J. Pharmacol. Exp. Ther. 290: 1467-1474, 1999). MGMT^{P140K}-based drug resistant gene therapy has been shown to confer chemoprotection to mouse, canine, rhesus macaques, and human cells, specifically hematopoietic cells (Zielske et al. J. Clin. Invest. 112: 1561-1570, 2003; Pollok et al. Hum. Gene Ther. 14: 1703-1714, 2003; Gerull et al. Hum. Gene Ther. 18: 451-456, 2007; Neff et al. Blood 105: 997-1002, 2005; Larochelle et al. Clin. Invest. 119: 1952-1963, 2009; Sawai et al. Mol. Ther. 3: 78-87, 2001).

In particular embodiments, combination with an in vivo selection marker will be a critical component for diseases without a selective advantage of gene-corrected cells. For example, in SCID and some other immunodeficiencies and FA, corrected cells have an advantage and only transducing the therapeutic gene into a “few” HSPCs is sufficient for therapeutic efficacy. For other diseases like hemoglobinopathies (i.e., sickle cell disease and thalassemia) in which cells do not demonstrate a competitive advantage, in vivo selection of the gene corrected cells, such as in combination with an in vivo selection marker such as MGMT^{P140K}, will select for the few transduced HSPCs, allowing an increase in the gene corrected cells and in order to achieve therapeutic efficacy. This approach can also be applied to HIV by making HSPCs resistant to HIV in vivo rather than ex vivo genetic modification.

Additional approaches can also be used. For example, the current disclosure can utilize systems and methods to genetically modify cells to provide a therapeutic gene while at the same time reducing CD33 expression selectively in the genetically modified therapeutic cells. In this manner, genetically modified therapeutic cells will not be harmed by concurrent or subsequent anti-CD33 therapies a patient may receive. However, pre-existing CD33-expressing cells in the patient and/or administered cells that lack the genetic modification will not be protected, resulting in positive selection for the gene-corrected cells over uncorrected cells.

In particular embodiments, this approach is achieved by linking the therapeutic gene and a CD33 blocking molecule in a single intracellular delivery vehicle. In particular embodiments, the single intracellular delivery vehicle is an Ad35 viral vector.

In particular embodiments, the CD33 blocking molecule is an shRNA or siRNA CD33 blocking molecule combined with the therapeutic gene by inclusion within a common Ad35 viral vector. In particular embodiments, the CD33 blocking molecule is the shRNA sequence including SEQ ID NO: 187 or sequence including SEQ ID NO: 188.

CD33-targeting treatments include anti-CD33 antibodies, anti-CD33 immunotoxins, anti-CD33 antibody-drug conjugates, anti-CD33 antibody-radioisotope conjugates, anti-CD33 bi-specific antibodies, anti-CD33 BiTE® antibodies, anti-CD33 tri-specific antibodies, and/or anti-CD33 CAR.

### IV(B). In Vitro and Ex Vivo Gene Therapy

In vitro gene therapy includes use of a vector, genome, or system of the present disclosure in a method of introducing exogenous DNA into a host cell (such as a target cell) and/or a nucleic acid (such as a target nucleic acid, such as a target genome), where the host cell or nucleic acid is not present in a multicellular organism (e.g., in a laboratory). In some embodiments, a target cell or nucleic acid is derived from a multicellular organism, such as a mammal (e.g., a mouse, rat, human, or non-human primate). In vitro engineering of a cell derived from a multicellular organism can be referred to as ex vivo engineering, and can be used in ex vivo therapy. In various embodiments, methods and compositions of the present disclosure are utilized, e.g., as disclosed herein, to modify a target cell or nucleic acid derived from a first multicellular organism and the engineered target cell or nucleic acid is then administered to a second multicellular organism, such as a mammal (e.g., a mouse, rat, human, or non-human primate), e.g., in a method of adoptive cell therapy. In some instances, the first and second organisms are the same single subject organism. Return of in vitro engineered material to a subject from which the material was derived can be an autologous therapy. In some instances, the first and second organisms are different organisms (e.g., two organisms of the same species, e.g., two mice, two rats, two humans, or two non-human primates of the same species). Transfer of engineered material derived from a first subject to a second different subject can be an allogeneic therapy.

Ex vivo cell therapies can include isolation of stem, progenitor or differentiated cells from a patient or a normal donor, expansion of isolated cells ex vivo—with or without genetic engineering—and administration of the cells to a subject to establish a transient or stable graft of the infused cells and/or their progeny. Such ex vivo approaches can be used, for example, to treat an inherited, infectious or neoplastic disease, to regenerate a tissue or to deliver a therapeutic agent to a disease site. In various ex vivo therapies there is no direct exposure of the subject to the gene transfer vector, and the target cells of transduction can be selected, expanded and/or differentiated, before or after any genetic engineering, to improve efficacy and safety.

Ex vivo therapies include haematopoietic stem cell (HSC) transplantation (HCT). Autologous HSC gene therapy represents a therapeutic option for several monogenic diseases of the blood and the immune system as well as for storage disorders, and it may become a first-line treatment option for selected disease conditions. Another established cell and gene therapy application is adoptive immunotherapy, which exploits ex vivo expanded T cells, with or without genetic engineering to redirect their antigen specificity or to increase their safety profile, in order to harness the power of immune effector and regulatory cells for use against malignancies, infections and autoimmune diseases. A range of other types of somatic stem cells—in some cases involving genetic engineering—are showing promise for therapeutic applications, including epidermal and limbal stem cells, neural stem/progenitor cells (NSPCs), cardiac stem cells and multipotent stromal cells (MSCs).

Applications of ex-vivo therapy include reconstituting dysfunctional cell lineages. For inherited diseases characterized by a defective or absent cell lineage, the lineage can be regenerated by functional progenitor cells, derived either from normal donors or from autologous cells that have been subjected to ex vivo gene transfer to correct the deficiency. An example is provided by SCIDs, in which a deficiency in any one of several genes blocks the development of mature lymphoid cells. Transplantation of non-manipulated normal donor HSCs, which can allow generation of donor-derived functional haematopoietic cells of various lineages in the host, represents a therapeutic option for SCIDs, as well as many other diseases that affect the blood and immune system. Autologous HSC gene therapy, which can include replacing a functional copy of a defective gene in transplanted haematopoietic stem/progenitor cells (HSPCs) and, similarly to HCT, can provide a steady supply of functional progeny, may have several advantages, including reduced risk of graft versus host disease (GvHD), reduced risk of graft rejection, and reduced need for post-transplant immunosuppression.

Applications of ex-vivo therapy include augmenting therapeutic gene dosage. In some applications, HSC gene therapy may augment the therapeutic efficacy of allogenic HCT. Therapeutic gene dosage can be engineered to supra-normal levels in transplanted cells.

Applications of ex-vivo therapy include introducing novel function and targeting gene therapy. Ex vivo gene therapy can confer a novel function to HSCs or their progeny, such as establishing drug resistance to allow administration of a high-dose antitumor chemotherapy regime or establishing resistance to a pre-established infection with a virus, such as HIV, or other pathogen by expressing RNA-based agents (for example, ribozymes, RNA decoys, antisense RNA, RNA aptamers and small interfering RNA) and protein-based agents (for example, dominant-negative mutant viral proteins, fusion inhibitors and engineered nucleases that target the pathogen's genome).

Applications of ex-vivo therapy include enhancing immune responses. In neoplastic diseases, allogenic adaptive immune cell types, such as T cells, can recognize and kill cancer cells. Unfortunately, recognition of healthy tissues by alloreactive lymphocytes can also result in detrimental GvHD. Transfer of a suicide gene in donor lymphocytes allows their anti-tumor potential to be exploited, while taming their toxicity. In the autologous setting, lymphocytes with specificity directed against transformed or infected cells may be isolated from the patient's tissues and selectively expanded ex vivo Alternatively, they may be generated by transfer of a gene for a synthetic or chimeric antigen receptor that triggers the cell's response when it encounters transformed or infected cells. These approaches may potentiate an underlying host response to a tumor or infection, or induce it de novo.

### IV(C). Conditions Treatable by Gene Therapy

At least in part because adenoviral vectors of the present disclosure can be used in vivo, in vitro, or ex vivo for modification of host and/or target cells, and further because an adenoviral vector can include payloads encoding a wide variety of expression products, it will be clear from the present specification that various technologies provide herein have broad applicability and can be used to treat a wide variety of conditions. Examples of conditions treatable by administration of an adenoviral vector, genome, or system of the present disclosure include, without limitation, hemoglobinopathies, immunodeficiencies, point mutation conditions, cancers, protein deficiencies, infectious diseases, and inflammatory conditions.

In certain embodiments, vectors, genomes, systems and formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Therapeutically effective amounts include those that provide effective amounts, prophylactic treatments, and/or therapeutic treatments.

In particular embodiments, methods and formulations disclosed herein can be used to treat blood disorders. In particular embodiments, formulations are administered to subjects to treat hemophilia, β-thalassemia major, Diamond Blackfan anemia (DBA), paroxysmal nocturnal hemoglobinuria (PNH), pure red cell aplasia (PRCA), refractory anemia, severe aplastic anemia, and/or blood cancers such as leukemia, lymphoma, and myeloma.

Hemoglobinopathies represent a global health burden with disproportionate outcomes. Defects in hemoglobin proteins or in the expression of globin genes can result in diseases termed hemoglobinopathies. Hemoglobinopathies are amongst the most common genetic disorders world-wide.

Every year, 1.1 million births worldwide are at risk for hemoglobinopathies, affecting as many as 25 in every 1,000 births in geographic regions where malaria falciparum is prevalent, owing to a natural resistance to malaria infection conferred by hemoglobin (Hb) genetic variance. In developed regions, patients are at risk of iron overload from chronic transfusions. In underdeveloped regions, survival is significantly lower. For example, in Africa, childhood mortality is 40% in patients with hemoglobinopathies, compared to 16% in all children.

Mutations in the globin genes may generate an abnormal form of hemoglobin, as in sickle cell disease (SCD) and hemoglobin C, D, and E disease, or result in reduced production of the α or β polypeptides and thus an imbalance of the globin chains in the cell. These latter conditions are termed α- or β-thalassemias, depending on which globin chain is impaired. 5% of the world population carry a significant hemoglobin variant with the sickle cell mutation in the b-globin (HBB) gene (a glutamate to valine conversion; historically E6V, contemporaneously E7V) being by far the most common (40% of carriers). The high prevalence and severity of hemoglobin disorders presents a substantial burden, impacting not only the lives of those affected but also health-care systems, since lifelong patient care is costly.

There are two forms of hemoglobin, fetal (HbF), which includes two alpha (α) and two gamma (γ) chains, and adult (HbA), which includes two α and two beta (β) chains. The natural switch from HbF to HbA occurs shortly after birth and is regulated by transcriptional repression of γ globin genes by factors including a master regulator, bcl11a. Critically, a variety of clinical observations demonstrate that the severity of β-hemoglobinopathies such as sickle cell disease and β-thalassemia are ameliorated by increased production of HbF.

In particular embodiments, a therapeutically effective treatment induces or increases expression of HbF, induces or increases production of hemoglobin and/or induces or increases production of β-globin. In particular embodiments, a therapeutically effective treatment improves blood cell function, and/or increases oxygenation of cells.

In various embodiments, the present disclosure includes treatment of a blood disorder using an adenoviral donor vector of the present disclosure that includes a β-globin long LCR, a β-globin promoter, and a coding nucleic acid sequence that encodes a protein or agent for treatment of the blood disorder. In various embodiments, the blood disorder is thalassemia and the protein is a β-globin or γ-globin protein, or a protein that otherwise partially or completely functionally replaces β-globin or γ-globin. In various embodiments, the blood disorder is hemophilia and the protein is ET3 or a protein that otherwise partially or completely functionally replaces Factor VIII. In various embodiments, the blood disorder is a point mutation disease such as sickle cell anemia, and the agent is a gene editing protein.

ET3 can have the following amino acid sequence: SEQ ID NO 301. In various embodiments, a Factor VIII replacement protein can have an amino acid sequence having at least 70%, 75%, 80%, 85%, 90%, 91%, 92%, 93%, 94%, 95%, 96%, 97%, 98%, 99%, or 100% identity to the SEQ ID NO: 301

β-globin can have the following amino acid sequence: SEQ ID NO 302. In various embodiments, a β-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 302.

γ-globin can have the following amino acid sequence: SEQ ID NO 303. In various embodiments, a γ-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 303.

More than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

X-linked severe combined immunodeficiency (SCID-X1) is both a cellular and humoral immune depletion caused by mutations in the common gamma chain gene (γC), which result in the absence of T and natural killer (NK) lymphocytes and the presence of nonfunctional B lymphocytes. SCID-X1 is fatal in the first two years of life unless the immune system is reconstituted, for example, through bone marrow transplant (BMT) or gene therapy.

Because most individuals lack a matched donor for BMT or non-autologous gene therapy, haploidentical parental bone marrow depleted of mature T cells is often used; however, complications include graft versus host disease (GVHD), failure to make adequate antibodies hence requiring long-term immunoglobulin replacement, late loss of T cells due to failure to engraft hematopoietic stem and progenitor cells (HSPCs), chronic warts, and lymphocyte dysregulation.

Fanconi anemia (FA) is an inherited blood disorder that leads to bone marrow failure. It is characterized, in part, by a deficient DNA-repair mechanism. At least 20% of patients with FA develop cancers such as acute myeloid leukemias, and cancers of the skin, liver, gastrointestinal tract, and gynecological systems. The skin and gastrointestinal tumors are usually squamous cell carcinomas. The average age of patients who develop cancer is 15 years for leukemia, 16 years for liver tumors, and 23 years for other tumors.

A therapeutic gene can be selected to provide a therapeutically effective response against a condition that, in particular embodiments, is inherited. In particular embodiments, the condition can be Grave's Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Fanconi anemia (FA), Battens disease, adrenoleukodystrophy (ALD) or metachromatic leukodystrophy (MLD), muscular dystrophy, pulmonary alveolar proteinosis (PAP), pyruvate kinase deficiency, Schwachman-Diamond-Blackfan anemia, dyskeratosis congenita, cystic fibrosis, Parkinson's disease, Alzheimer's disease, or amyotrophic lateral sclerosis (Lou Gehrig's disease). In particular embodiments, depending on the condition, the therapeutic gene may be a gene that encodes a protein and/or a gene whose function has been interrupted.

In particular embodiments, methods and formulations disclosed herein can be used to treat cancer. In particular embodiments, formulations are administered to subjects to treat acute lymphoblastic leukemia (ALL), acute myelogenous leukemia (AML), chronic lymphocytic leukemia (CLL), chronic myelogenous leukemia (CML), chronic myelomonocytic leukemia, diffuse large B-cell lymphoma, follicular lymphoma, Hodgkin's lymphoma, juvenile myelomonocytic leukemia, multiple myeloma, myelodysplasia, and/or non-Hodgkin's lymphoma.

Additional exemplary cancers that may be treated include astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, clear cell sarcoma of soft tissue, diffuse large B-cell lymphoma, ependymoma, epithelioid sarcoma, extragonadal germ cell tumor, extrarenal rhabdoid tumor, Ewing sarcoma, gastrointestinal stromal tumor, glioblastoma, HBV-induced hepatocellular carcinoma, head and neck cancer, kidney cancer, lung cancer, malignant rhabdoid tumor, medulloblastoma, melanoma, meningioma, mesothelioma, multiple myeloma, neuroglial tumor, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullo carcinoma, rhabdomyosarcoma, sarcoma, schwannoma, skin squamous cell carcinoma, and stem cell cancer. In various particular embodiments, the cancer is ovarian cancer. In various particular embodiments the cancer is breast cancer.

In particular embodiments, methods and formulations disclosed herein can be used to treat point mutation conditions. In particular embodiments, formulations are administered to subjects to treat sickle cell disease, cystic fibrosis, Tay-Sachs disease, and/or phenylketonuria. In various embodiments, a transposon payload of the present disclosure encodes a CRISPR-Cas for corrective editing of a nucleic acid lesion. In various embodiments, a transposon payload of the present disclosure encodes a base editor for corrective editing of a nucleic acid lesion.

In particular embodiments, methods and formulations disclosed herein can be used to treat particular enzyme deficiency. In particular embodiments, formulations are administered to subjects to treat Hurler's syndrome, selective IgA deficiency, hyper IgM, IgG subclass deficiency, Niemann-Pick disease, Tay-Sachs disease, Gaucher disease, Fabry disease, Krabbe disease, glucosemia, maple syrup urine disease, phenylketonuria, glycogen storage disease, Friedreich ataxia, Zellweger syndrome, adrenoleukodystrophy, complement disorders, and/or mucopolysaccharidoses.

Therapeutically effective amounts may provide function to immune and other blood cells and/or microglial cells or may alternatively—depending on the treated condition—inhibit lymphocyte activation, induce apoptosis in lymphocytes, eliminate various subsets of lymphocytes, inhibit T cell activation, eliminate or inhibit autoreactive T cells, inhibit Th-2 or Th-1 lymphocyte activity, antagonize IL-1 or TNF, reduce inflammation, induce selective tolerance to an inciting agent, reduce or eliminate an immune-mediated condition; and/or reduce or eliminate a symptom of the immune-mediated condition. Therapeutically effective amounts may also provide functional DNA repair mechanisms; surfactant protein expression; telomere maintenance; lysosomal function; breakdown of lipids or other proteins such as amyloids; permit ribosomal function; and/or permit development of mature blood cell lineages which would otherwise not develop such as macrophages other white blood cell types.

In particular embodiments, methods of the present disclosure can restore T-cell mediated immune responses in a subject in need thereof. Restoration of T-cell mediated immune responses can include restoring thymic output and/or restoring normal T lymphocyte development.

In particular embodiments, restoring thymic output can include restoring the frequency of CD3+ T cells expressing CD45RA in peripheral blood to a level comparable to that of a reference level derived from a control population. In particular embodiments, restoring thymic output can include restoring the number of T cell receptor excision circles (TRECs) per 106 maturing T cells to a level comparable to that of a reference level derived from a control population. The number of TRECs per 106 maturing T cells can be determined as described in Kennedy et al., Vet Immunol Immunopathol 142: 36-48, 2011.

In particular embodiments, restoring normal T lymphocyte development includes restoring the ratio of CD4+ cells: CD8+ cells to 2. In particular embodiments, restoring normal T lymphocyte development includes detecting the presence of αβ TCR in circulating T-lymphocytes. The presence of αβ TCR in circulating T-lymphocytes can be detected, for example, by flow cytometry using antibodies that bind an α and/or β chain of a TCR. In particular embodiments, restoring normal T lymphocyte development includes detecting the presence of a diverse TCR repertoire comparable to that of a reference level derived from a control population. TCR diversity can be assessed by TCRVβ spectratyping, which analyzes genetic rearrangement of the variable region of the TCR gene. Robust, normal spectratype profiles can be characterized by a Gaussian distribution of fragments sized across 17 families of TCRVβ segments. In particular embodiments, restoring normal T lymphocyte development includes restoring T-cell specific signaling pathways. Restoration of T-cell specific signaling pathways can be assessed by lymphocyte proliferation following exposure to the T cell mitogen phytohemagglutinin (PHA). In particular embodiments, restoring normal T lymphocyte development includes restoring white blood cell count, neutrophil cell count, monocyte cell count, lymphocyte cell count, and/or platelet cell count to a level comparable to a reference level derived from a control population.

In particular embodiments, methods of the present disclosure can improve the kinetics and/or clonal diversity of lymphocyte reconstitution in a subject in need thereof. In particular embodiments, improving the kinetics of lymphocyte reconstitution can include increasing the number of circulating T lymphocytes to within a range of a reference level derived from a control population. In particular embodiments, improving the kinetics of lymphocyte reconstitution can include increasing the absolute CD3+ lymphocyte count to within a range of a reference level derived from a control population. A range of can be a range of values observed in or exhibited by normal (i.e., non-immuno-compromised) subjects for a given parameter. In particular embodiments, improving the kinetics of lymphocyte reconstitution can include reducing the time required to reach normal lymphocyte counts as compared to a subject in need thereof not administered a therapy described herein. In particular embodiments, improving the kinetics of lymphocyte reconstitution can include increasing the frequency of gene corrected lymphocytes as compared to a subject in need thereof not administered a therapy described herein. In particular embodiments, improving the kinetics of lymphocyte reconstitution can include increasing diversity of clonal repertoire of gene corrected lymphocytes in the subject as compared to a subject in need thereof not administered a gene therapy described herein. Increasing diversity of clonal repertoire of gene corrected lymphocytes can include increasing the number of unique retroviral integration site (RIS) clones as measured by a RIS analysis.

In particular embodiments, methods of the present disclosure can restore bone marrow function in a subject in need thereof. In particular embodiments, restoring bone marrow function can include improving bone marrow repopulation with gene corrected cells as compared to a subject in need thereof not administered a therapy described herein. Improving bone marrow repopulation with gene corrected cells can include increasing the percentage of cells that are gene corrected. In particular embodiments, the cells are selected from white blood cells and bone marrow derived cells. In particular embodiments, the percentage of cells that are gene corrected can be measured using an assay selected from quantitative real time PCR and flow cytometry.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage ψX174. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, methods of the present disclosure can be used to treat SCID-X1. In particular embodiments, methods of the present disclosure can be used to treat SCID (e.g., JAK 3 kinase deficiency SCID, purine nucleoside phosphorylase (PNP) deficiency SCID, adenosine deaminase (ADA) deficiency SCID, MHC class II deficiency or recombinase activating gene (RAG) deficiency SCID). In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating SCIDX-1 with methods of the present disclosure include restoring functionality to the γC-dependent signaling pathway. The functionality of the γC-dependent signaling pathway can be assayed by measuring tyrosine phosphorylation of effector molecules STAT3 and/or STAT5 following in vitro stimulation with IL-21 and/or IL-2, respectively. Tyrosine phosphorylation of STAT3 and/or STAT5 can be measured by intracellular antibody staining.

In particular embodiments, methods of the present disclosure can be used to treat FA. In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating FA with methods of the present disclosure include increasing resistance of bone marrow derived cells to mitomycin C (MMC). In particular embodiments, the resistance of bone marrow derived cells to MMC can be measured by a cell survival assay in methylcellulose and MMC.

In particular embodiments, methods of the present disclosure can be used to treat hypogammaglobulinemia. Hypogammaglobulinemia is caused by a lack of B-lymphocytes and is characterized by low levels of antibodies in the blood. Hypogammaglobulinemia can occur in patients with chronic lymphocytic leukemia (CLL), multiple myeloma (MM), non-Hodgkin's lymphoma (NHL) and other relevant malignancies as a result of both leukemia-related immune dysfunction and therapy-related immunosuppression. Patients with acquired hypogammaglobulinemia secondary to such hematological malignancies, and those patients receiving post-HSPC transplantation are susceptible to bacterial infections. The deficiency in humoral immunity is largely responsible for the increased risk of infection-related morbidity and mortality in these patients, especially by encapsulated microorganisms. For example, Streptococcus pneumoniae, Haemophilus influenzae, and Staphylococcus aureus, as well as Legionella and Nocardia spp. are frequent bacterial pathogens that cause pneumonia in patients with CLL. Opportunistic infections such as Pneumocystis carinii, fungi, viruses, and mycobacteria also have been observed. The number and severity of infections in these patients can be significantly reduced by administration of immune globulin (Griffiths et al. Blood 73: 366-368, 1989; Chapel et al. Lancet 343: 1059-1063, 1994).

In particular embodiments, formulations are administered to subjects to treat acute lymphoblastic leukemia (ALL), acute myelogenous leukemia (AML), adrenoleukodystrophy, agnogenic myeloid metaplasia, amegakaryocytosic/congenital thrombocytopenia, ataxia telangiectasia, β-thalassemia major, chronic granulomatous disease, chronic lymphocytic leukemia (CLL), chronic myelogenous leukemia (CML), chronic myelomonocytic leukemia, common variable immune deficiency (CVID), complement disorders, congenital agammaglobulinemia, Diamond Blackfan syndrome, diffuse large B-cell lymphoma, familial erythrophagocytic lymphohistiocytosis, follicular lymphoma, Hodgkin's lymphoma, Hurler's syndrome, hyper IgM, IgG subclass deficiency, juvenile myelomonocytic leukemia, metachromatic leukodystrophy, mucopolysaccharidoses, multiple myeloma, myelodysplasia, non-Hodgkin's lymphoma, paroxysmal nocturnal hemoglobinuria (PNH), primary immunodeficiency diseases with antibody deficiency, pure red cell aplasia, refractory anemia, Shwachman-Diamond-Blackfan anemia (DBA), selective IgA deficiency, severe aplastic anemia, sickle cell disease, specific antibody deficiency, Wiskott-Aldridge syndrome, and/or X-linked agammaglobulinemia (XLA).

Additional exemplary cancers that may be treated include astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, clear cell sarcoma of soft tissue, diffuse large B-cell lymphoma, ependymoma, epithelioid sarcoma, extragonadal germ cell tumor, extrarenal rhabdoid tumor, Ewing sarcoma, gastrointestinal stromal tumor, glioblastoma, HBV-induced hepatocellular carcinoma, head and neck cancer, kidney cancer, lung cancer, malignant rhabdoid tumor, medulloblastoma, melanoma, meningioma, mesothelioma, multiple myeloma, neuroglial tumor, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullary carcinoma, rhabdomyosarcoma, sarcoma, schwannoma, skin squamous cell carcinoma, and stem cell cancer. In various particular embodiments, the cancer is ovarian cancer. In various particular embodiments the cancer is breast cancer.

In the context of cancers, therapeutically effective amounts can decrease the number of tumor cells, decrease the number of metastases, decrease tumor volume, increase life expectancy, induce apoptosis of cancer cells, induce cancer cell death, induce chemo- or radiosensitivity in cancer cells, inhibit angiogenesis near cancer cells, inhibit cancer cell proliferation, inhibit tumor growth, prevent metastasis, prolong a subject's life, reduce cancer-associated pain, reduce the number of metastases, and/or reduce relapse or re-occurrence of the cancer following treatment.

Particular embodiments include treatment of secondary, or acquired, immune deficiencies such as immune deficiencies caused by trauma, viruses, chemotherapy, toxins, and pollution. As previously indicated, acquired immunodeficiency syndrome (AIDS) is an example of a secondary immune deficiency disorder caused by a virus, the human immunodeficiency virus (HIV), in which a depletion of T lymphocytes renders the body unable to fight infection. Thus, as another example, a gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/a2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

Particular embodiments, formulations are administered to subjects to prevent or delay cancer reoccurrence or prevent or delay cancer onset in carriers of high-risk germ line mutations. In particular embodiments, formulations are administered to subjects to receive higher therapeutic doses of temozolomide (TMZ) and benzylguanine or BCNU. Due to strong myelosupressvive off-target effects, it remains a challenge to deliver an effective dose of TMZ and benzylguanine to tumors. Patients may currently receive TMZ and benzylguanine for treatments associated with acute myeloid leukemia (AML), esophageal Cancer, Head & Neck Cancer, High-Grade Glioma, myelodysplastic syndrome, non-small cell lung cancer, NSCLC; Refractory AML, small cell lung cancer, anaplastic astrocytoma, brain tumors, breast cancer (e.g., metastatic), colorectal cancer (e.g., metastatic), diffuse intrinsic brainstem glioma, Ewing sarcoma, glioblastoma multiforme (GBM), malignant glioma, melanoma, metastatic malignant melanoma, recurrent malignant melanoma, nasopharyngeal cancer, metastatic breast cancer, and pediatric cancers.

Patients with MGMT expressing tumors would benefit from administration of Ad35 viral vector with an active ingredient (such as a CAR, TCR, or checkpoint inhibitor) combined with the MGMT^{P140K} in vivo selection cassette. Ex vivo approaches have shown the applicability of this approach. In particular embodiments, therapeutic amounts of TMZ and benzylguanine or BCNU are administered to reduce the tumor burden or volume.

In particular embodiments, therapeutically effective amounts may provide function to immune and other blood cells, reduce or eliminate an immune-mediated condition; and/or reduce or eliminate a symptom of the immune-mediated condition.

In the vectors, mobilization factors, formulations, and methods of use described herein, variants of protein and/or nucleic acid sequences can also be used. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

Obtained values for parameters associated with in vivo gene therapy and/or HSPC mobilization described herein can be compared to a reference level derived from a control population, and this comparison can indicate whether an in vivo gene therapy described herein is effective for a subject in need thereof administered the gene therapy. Parameters associated with in vivo gene therapy and/or HSPC mobilization can include, for example: number of total white blood cells, neutrophils, monocytes, lymphocytes, and/or platelets; time required to reach normal lymphocyte counts; percent CD3+CD45RA+ T cells; number of TRECs per 10⁶ cells; percent of cells that are CD4+; percent of cells that are CD8+; the ratio of CD4/CD8; percent of TCRαβ+ cells in CD3+ T cells; diversity of TCR; frequency of gene corrected lymphocytes; diversity of clonal repertoire of gene corrected lymphocytes; number of unique RIS clones; primary and secondary antibody responses to bacteriophage injection; rate of bacteriophage inactivation; percentage of cells that are gene corrected; level of immunoglobulins IgA, IgM, and/or IgG; resistance of bone marrow derived cells to mitomycin C; percent of living cells in methylcellulose and mitomycin C; functionality of γC-dependent signaling pathway; and percent phosphorylation of STAT3 with IL-21/mitogen stimulation of cells. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual datapoints; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 0-2 years) and non-immunocompromised status. In particular embodiments, a normal control population includes individuals that are age-matched to a test subject and non-immune compromised. In particular embodiments, age-matched includes, e.g., 0-6 months old; 0-1 year old; 0-2 years old; 0-3 years old; 10-15 years old, as is clinically relevant under the circumstances).

In particular embodiments, the relevant reference level for values of a particular parameter associated with in vivo gene therapy and/or HSPC mobilization described herein is obtained based on the value of a particular corresponding parameter associated with in vivo gene therapy and/or HSPC mobilization in a control population to determine whether an in vivo gene therapy disclosed herein has been therapeutically effective for a subject in need thereof administered the gene therapy.

In particular embodiments, a control population can include those that are healthy and do not have immune deficiencies. In particular embodiments, a control population can include those that have an immune deficiency and have not been administered a therapeutically effective amount of (i) a formulation including an Ad35 viral vector associated with a therapeutic gene; and (ii) mobilization factors. In particular embodiments, a control population can include those that have an immune deficiency and have been administered a therapeutically effective amount of a formulation including an Ad35 viral vector associated with a therapeutic gene and not including mobilization factors. As an example, the relevant reference level can be the value of the particular parameter associated with in vivo gene therapy and/or HSPC mobilization in the control subjects.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

In particular embodiments, values obtained for parameters associated with in vivo gene therapy and/or HSPC mobilization described herein and/or other dataset components can be subjected to an analytic process with chosen parameters. The parameters of the analytic process may be those disclosed herein or those derived using the guidelines described herein. The analytic process used to generate a result may be any type of process capable of providing a result useful for classifying a sample, for example, comparison of the obtained value with a reference level, a linear algorithm, a quadratic algorithm, a decision tree algorithm, or a voting algorithm. The analytic process may set a threshold for determining the probability that a sample belongs to a given class. The probability preferably is at least 60%, at least 70%, at least 80%, at least 90%, at least 95% or higher.

Ad35 vectors described herein can be utilized in place of Ad5/Ad35++ vectors described in the following Exemplary Embodiments and Examples.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### V. EXEMPLARY EMBODIMENTS

A first set of exemplary embodiments can include the following:

1. A recombinant adenoviral serotype 35 (Ad35) vector production system including: a recombinant Ad35 helper genome including: a nucleic acid sequence encoding an Ad35 fiber shaft; a nucleic acid sequence encoding an Ad35 fiber knob; and recombinase direct repeats (DRs) flanking at least a portion of an Ad35 packaging sequence, and a recombinant helper dependent Ad35 donor genome including: a 5′ Ad35 inverted terminal repeat (ITR); a 3′ Ad35 ITR; an Ad35 packaging sequence; and a nucleic acid sequence encoding at least one heterologous expression product.
2. A recombinant adenoviral serotype 35 (Ad35) helper vector including: an Ad35 fiber shaft; an Ad35 fiber knob; and an Ad35 genome including recombinase direct repeats (DRs) flanking at least a portion of an Ad35 packaging sequence.
3. A recombinant adenoviral serotype 35 (Ad35) helper genome including: a nucleic acid sequence encoding an Ad35 fiber shaft; a nucleic acid sequence encoding an Ad35 fiber knob; and recombinase direct repeats (DRs) flanking at least a portion of an Ad35 packaging sequence.
4. A recombinant helper dependent adenoviral serotype 35 (Ad35) donor vector including: a nucleic acid sequence including: a 5′ Ad35 inverted terminal repeat (ITR); a 3′ Ad35 ITR; an Ad35 packaging sequence; and a nucleic acid sequence encoding at least one heterologous expression product, wherein the genome does not include a nucleic acid sequence encoding an Ad35 viral structural protein; and an Ad35 fiber shaft and/or an Ad35 fiber knob.
5. A recombinant helper dependent adenoviral serotype 35 (Ad35) donor genome including: a 5′ Ad35 inverted terminal repeat (ITR); a 3′ Ad35 ITR; an Ad35 packaging sequence; and a nucleic acid sequence encoding at least one heterologous expression product, wherein the Ad35 donor genome does not include a nucleic acid sequence encoding an expression product encoded by the wild-type Ad35 genome.
6. A method of producing a recombinant helper dependent adenoviral serotype 35 (Ad35) donor vector, the method including isolating the recombinant helper dependent Ad35 donor vector from a culture of cells, wherein the cells include: a recombinant Ad35 helper genome including: a nucleic acid sequence encoding an Ad35 fiber shaft; a nucleic acid sequence encoding an Ad35 fiber knob; and recombinase direct repeats (DRs) flanking at least a portion of an Ad35 packaging sequence, and a recombinant helper dependent Ad35 donor genome including: a 5′ Ad35 inverted terminal repeat (ITR); a 3′ Ad35 ITR; an Ad35 packaging sequence; and a nucleic acid sequence encoding at least one heterologous expression product.
7. A recombinant adenoviral serotype 35 (Ad35) production system including: a recombinant Ad35 helper genome including: a nucleic acid sequence encoding an Ad35 fiber shaft; a nucleic acid sequence encoding an Ad35 fiber knob; and recombinase direct repeats (DRs) within 550 nucleotides of the 5′ end of the Ad35 genome that functionally disrupt the Ad35 packaging signal but not the 5′ Ad35 inverted terminal repeat (ITR), and a recombinant Ad35 donor genome including: a 5′ Ad35 ITR; a 3′ Ad35 ITR; an Ad35 packaging sequence; and a nucleic acid sequence encoding at least one heterologous expression product.
8. A recombinant adenoviral serotype 35 (Ad35) helper vector including: an Ad35 fiber shaft; an Ad35 fiber knob; and an Ad35 genome including recombinase direct repeats (DRs) within 550 nucleotides of the 5′ end of the Ad35 genome that functionally disrupt the Ad35 packaging signal but not the 5′ Ad35 inverted terminal repeat (ITR).
9. A recombinant adenoviral serotype 35 (Ad35) helper genome including: a nucleic acid sequence encoding an Ad35 fiber shaft; a nucleic acid sequence encoding an Ad35 fiber knob; and recombinase direct repeats (DRs) within 550 nucleotides of the 5′ end of the Ad35 genome that functionally disrupt the Ad35 packaging signal but not the 5′ Ad35 inverted terminal repeat (ITR).
10. A method of producing a recombinant helper dependent adenoviral serotype 35 (Ad35) donor vector, the method including isolating the recombinant helper dependent Ad35 donor vector from a culture of cells, wherein the cells include: a recombinant Ad35 helper genome including: a nucleic acid sequence encoding an Ad35 fiber shaft; a nucleic acid sequence encoding an Ad35 fiber knob; and recombinase direct repeats (DRs) within 550 nucleotides of the 5′ end of the Ad35 genome that functionally disrupt the Ad35 packaging signal but not the 5′ Ad35 inverted terminal repeat (ITR), and a recombinant Ad35 donor genome including: a 5′ Ad35 ITR; a 3′ Ad35 ITR; an Ad35 packaging sequence; and a nucleic acid sequence encoding at least one heterologous expression product.
11. The recombinant Ad35 vector production system, helper vector, helper genome, donor vector, or method of any one of embodiments 1˜4 or 6-10, wherein: the Ad35 fiber knob includes a wild-type Ad35 fiber knob, or the Ad35 fiber knob includes an engineered Ad35 fiber knob, wherein the engineered fiber knob includes a mutation that increases affinity of the fiber knob with CD46.
12. The recombinant Ad35 vector production system, helper vector, helper genome, donor vector, or method of embodiment 11, wherein the mutation: includes a mutation selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or includes each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.
13. The recombinant Ad35 vector production system, donor genome, donor vector, or method of any one of embodiments 1, 4-7, or 10-12, wherein the heterologous expression product includes a therapeutic expression product operably linked with a regulatory sequence, optionally wherein the therapeutic expression product includes: (a) a β-globin protein or γ-globin protein; (b) an antibody or an immunoglobulin chain thereof, optionally wherein the antibody includes an anti-CD33 antibody; (c) a first antibody or an immunoglobulin chain thereof and a second antibody or an immunoglobulin chain thereof, optionally wherein the antibody includes an anti-CD33 antibody; (d) a CRISPR-associated RNA-guided endonuclease and/or a guide RNA (gRNA), optionally wherein the CRISPR-associated RNA-guided endonuclease includes Cas9 or cpf1; (e) a base editor and/or a gRNA, optionally wherein the base editor includes a cytosine base editor (CBE) or adenine base editor (ABE), optionally wherein the base editor includes a catalytically disabled nuclease selected from a disabled Cas9 and a disabled cpf1; (f) a coagulation factor or a protein that blocks or reduces viral infection, optionally wherein the therapeutic expression produce includes a Factor VII replacement protein or a Factor VIII replacement protein; (g) a checkpoint inhibitor; (h) chimeric antigen receptor or engineered T cell receptor; or (i) a protein selected from the group consisting of γC, JAK3, IL7RA, RAG1, RAG2, DCLRE1C, PRKDC, LIG4, NHEJ1, CD3D, CD3ε, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, FancA, FancB, FancC, FancD1, FancD2, FancE, FancF, FancG, Fancl, FancJ, FancL, FancM, FancN, FancO, FancP, FancQ, FancR, FancS, FancT, FancU, FancV, FancW, soluble CD40, CTLA, Fas L, an antibody to PD-L1, an antibody to CD4, an antibody to CD5, an antibody to CD7, an antibody to CD52, an antibody to IL-1, an antibody to IL-2, an antibody to IL-4, an antibody to IL-6, an antibody to IL-10, an antibody to TNF, an antibody to a TCR specifically present on autoreactive T cells, a globin family gene, WAS, phox, dystrophin, pyruvate kinase, CLN3, ABCD1, arylsulfatase A, SFTPB, SFTPC, NLX2.1, ABCA3, GATA1, a ribosomal protein gene, TERT, TERC, DKC1, TINF2, CFTR, LRRK2, PARK2, PARK7, PINK1, SNCA, PSEN1, PSEN2, APP, SOD1, TDP43, FUS, ubiquilin 2, and/or C9ORF72, optionally wherein the protein includes a FancA protein.
14. The recombinant Ad35 vector production system, donor genome, donor vector, or method of embodiment 13(d) or 13(e), wherein: the gRNA binds a target nucleic acid sequence of HBG1, HBG2, and/or erythroid enhancer bcl11a, optionally wherein the gRNA is engineered to increase expression of γ-globin; or the gRNA binds a target nucleic acid sequence that encodes a portion of CD33, optionally wherein the CD33 includes human CD33.
15. The recombinant Ad35 vector production system, donor genome, donor vector, or method of embodiment 13, wherein the therapeutic expression product includes: a β-globin protein or a γ-globin protein; and a CRISPR system including a CRISPR-associated RNA-guided endonuclease; and one, two, or three of: a gRNA that binds a target nucleic acid sequence of HBG1; a gRNA that binds a target nucleic acid sequence of HBG2; and/or a gRNA that binds a target nucleic acid sequence of Bcl11a, optionally wherein the gRNA is engineered to increase expression of γ-globin.
16. The recombinant Ad35 vector production system, donor genome, donor vector, or method of embodiment 13, wherein the regulatory sequence(s) include a promoter, optionally wherein the promoter includes a β-globin promoter, optionally wherein the β-globin promoter has a length of about 1.6 kb and/or includes a nucleic acid according to positions 5228631-5227023 of chromosome 11.
17. The recombinant Ad35 vector production system, donor genome, donor vector, or method of embodiment 13, wherein the regulatory sequence(s) include a Locus Control Region (LCR), optionally wherein the LCR includes a β-globin LCR
18. The recombinant Ad35 vector production system, donor genome, donor vector, or method of embodiment 13, wherein the β-globin LCR:
includes β-globin LCR DNAse I hypersensitive sites (HS) including or consisting of HS1, HS2, HS3, and HS4, optionally wherein the β-globin LCR has a length of about 4.3 kb;
includes β-globin LCR DNAse I HS including HS1, HS2, HS3, HS4, and HS5, optionally wherein the β-globin LCR has a length of about 21.5 kb; or wherein the β-globin LCR includes a sequence according to positions 5292319-5270789 of chromosome 11.
19. The recombinant Ad35 vector production system, donor genome, donor vector, or method of embodiment 13 or 14, wherein the regulatory sequence(s) include a 3′HS1, optionally wherein the 3′HS1 includes a sequence according to positions 5206867-5203839 of chromosome 11.
20. The recombinant Ad35 vector production system, donor genome, donor vector, or method of any one of embodiments 13-19, wherein the regulatory sequence(s) include an miRNA binding site, optionally wherein: the miRNA binding site includes a binding site for an miRNA naturally expressed by a species of interest; the miRNA demonstrates differential occupancy profiles in the blood and a tumor microenvironment or target tissue, optionally wherein the occupancy profile is higher in blood than in the tumor microenvironment or target tissue; the miRNA binding site includes an miR423-5, miR423-5p, miR42-2, miR181c, miR125a, or miR15a binding sites; and/or the miRNA binding sites include an miR187 or miR218 binding sites.
21. The recombinant Ad35 vector production system, donor genome, donor vector, or method of any one of embodiments 1, 4-7, or 10-21, wherein the nucleic acid encoding the heterologous expression product is part of a payload further including an integration element, optionally wherein the integration element includes an expression product.
22. The recombinant Ad35 vector production system, donor genome, donor vector, or method of embodiment 21, wherein the integration element is engineered for integration into a target genome by homologous recombination, wherein the integration element is flanked by homology arms that correspond to contiguously linked sequences of the target genome, optionally wherein: the homology arms are between 0.8 and 1.8 kb; and/or the homology arms are homologous to nucleic acid sequences of the target genome that flank a chromosomal safe harbor locus, optionally wherein the safe harbor loci is selected from AAVS1, CCR5, HPRT, or Rosa.
23. The recombinant Ad35 vector production system, donor genome, donor vector, or method of embodiment 21, wherein the integration element is engineered for integration into a target genome by transposition, wherein the integration element is flanked by transposon inverted repeats (IRs), optionally wherein the transposon IRs are flanked by recombinase DRs.
24. The recombinant Ad35 vector production system, donor genome, donor vector, or method of embodiment 23, wherein: the transposon IRs are Sleeping Beauty (SB) IRs, optionally wherein the SB IRs are pT4 IRs; or the transposon IRs are piggyback, Mariner, frog prince, Tol2, TcBuster, or spinON IRs.
25. The recombinant Ad35 vector production system, donor genome, donor vector, or method of any one of embodiments 21-24, including a nucleic acid encoding a transposase that mediates transposition of the integration element flanked by the transposon IRs, optionally wherein the nucleic acid encoding the transposase is comprised by a support vector or support vector genome.
26. The recombinant Ad35 vector production system, donor genome, donor vector, or method of embodiment 25, wherein the transposase includes a Sleeping beauty, piggyback, Mariner, frog prince, Tol2, TcBuster, or spinON transposase, optionally wherein the transposase includes a Sleeping Beauty 100x (SB100x) transposase.
27. The recombinant Ad35 vector production system, donor genome, donor vector, or method of embodiment 25 or 26, wherein the nucleic acid encoding the transposase is operably linked with a PGK promoter.
28. The recombinant Ad35 vector production system, helper vector, helper genome, or method of any one of embodiments 1-3 or 6-27, wherein the recombinase DRs that flank at least a portion of the Ad35 packaging sequence and/or are within 550 nucleotides of the 5′ end of the Ad35 genome and functionally disrupt the Ad35 packaging signal but not the 5′ Ad35 ITR are FRT, loxP, rox, vox, AttB, or AttP sites.
29. The recombinant Ad35 vector production system, helper vector, helper genome, or method of embodiment 28, wherein a nucleic acid encoding a recombinase for excision of the at least portion of the Ad35 packaging sequence is encoded by a nucleic acid sequence of a cell including the helper genome.
30. The recombinant Ad35 vector production system, helper vector, helper genome, or method of any one of embodiments 23-29, wherein the recombinase DRs that flank the transposon IRs are FRT, loxP, rox, vox, AttB, or AttP sites.
31. The recombinant Ad35 vector production system, helper vector, helper genome, or method of any one of embodiments 21-28, wherein a nucleic acid encoding a recombinase for excision of the nucleic acid including the integration element is comprised by a support vector or support vector genome.
32. The recombinant Ad35 vector production system, helper vector, helper genome, or method of embodiment 29 or 31, wherein the recombinase includes a Flp, Cre, Dre, Vika, or PhiC31 recombinase.
33. The recombinant Ad35 vector production system, helper vector, helper genome, or method of embodiment 32, wherein the nucleic acid encoding the recombinase is operably linked with an EF1α promoter.
34. The recombinant Ad35 vector production system, helper vector, helper genome, or method of any one of embodiments 21-33, wherein the payload includes an integration element including the heterologous expression product, wherein the heterologous expression product includes a β-globin protein operably linked with a β-globin promoter and a β-globin long LCR, wherein the integration element is flanked by SB IRs, and wherein the SB IRs are flanked by recombinase DRs, optionally wherein the recombinase DRs are FRT sites.
35. The recombinant Ad35 vector production system, helper vector, helper genome, or method of any one of embodiments 21-34, wherein the payload includes: an integration element, and a conditionally expressed nucleic acid sequence that encodes an expression product, is not comprised by the integration element, and is positioned such that it is rendered nonfunctional by integration of the integration element into a target genome.
36. The recombinant Ad35 vector production system, helper vector, helper genome, or method of embodiment 35, wherein the expression product encoded by the conditionally expressed nucleic acid sequence includes a CRISPR system component or a base editor system component, optionally wherein the component includes a CRISPR-associated RNA-guided endonuclease, a base editor enzyme, or a gRNA.
37. The recombinant Ad35 vector production system, helper vector, helper genome, or method of any one of embodiments 21-36, wherein the payload includes a selection cassette, optionally wherein the selection cassette is comprised by the integration element.
38. The recombinant Ad35 vector production system, helper vector, helper genome, or method of embodiment 37, wherein the selection cassette includes a nucleic acid sequence encoding mgmt^{P140K} or wherein the selection cassette includes a nucleic acid sequence encoding an anti-CD33 shRNA.
39. The recombinant Ad35 vector production system, helper vector, helper genome, or method of any one of embodiments 1-3 or 6-38, wherein the at least portion of the Ad35 packaging sequence flanked by recombinase DRs corresponds to nucleotides 138-481 of the Ad35 sequence according to Gen Bank Accession No. AX049983.
40. The recombinant Ad35 vector production system, helper vector, helper genome, or method of any one of embodiments 1-3 or 6-38, wherein the at least portion of the Ad35 packaging sequence flanked by recombinase DRs corresponds to: nucleotides 179-344; nucleotides 366-481; nucleotides 155-481; nucleotides 159-480; nucleotides 159-446; nucleotides 180-480; nucleotides 207-480; nucleotides 140-446; nucleotides 159-446; nucleotides 180-446; nucleotides 202-446; nucleotides 159-481; nucleotides 180-384; nucleotides 180-481; or nucleotides 207-481. of the Ad35 sequence according to Gen Bank Accession No. AX049983.
41. The recombinant Ad35 vector production system, helper vector, helper genome, or method of any one of embodiments 1-3 or 6-40, wherein the recombinase DRs are LoxP sites.
42. The helper vector or helper genome of any one of embodiments 2, 3, 8, or 9, wherein the Ad35 helper genome includes Ad5 E4orf6 for amplification in 293 T cells.
43. The helper vector or helper genome of any one of embodiments 2, 3, 8, or 9, wherein the helper genome includes or generates the sequence as set forth in any one of SEQ ID NOs: 51-65.
44. A cell including the helper vector, the helper genome, the donor vector, or the donor genome of any one of embodiments 2-5, 8, or 9, optionally wherein the cell is a HEK293 cell.
45. A cell including the donor genome of any one of embodiments 1, 4, 6, 7, 10, 13-27 or 44 optionally wherein the cell is an erythrocyte, optionally wherein the cell is a hematopoietic stem cell, T-cell, B-cell, or myeloid cell, optionally wherein the cell secretes the expression product.
46. The method of any one of embodiments 6 or 10-41, wherein the cells are HEK293 cells.
47. A method of modifying a cell, the method including contacting the cell with an Ad35 donor vector according to any one of embodiments 5 or 11-27.
48. A method of modifying a cell of a subject, the method including administering to the subject an Ad35 donor vector according to any one of embodiments 5 or 11-27, optionally wherein the method does not include isolation of the cell from the subject.
49. A method of treating a disease or condition in a subject in need thereof, the method including administering to the subject an Ad35 donor vector according to any one of embodiments 5 or 11-27, optionally wherein the administration is intravenous.
50. The method of embodiment 49, wherein the method includes administering to the subject a mobilization agent, optionally wherein the mobilization agent includes one or more of granulocyte-colony stimulating factor, GM-CSF, S-CSF, a CXCR4 antagonist, and a CXCR2 agonist, optionally wherein the CXCR4 antagonist includes AMD3100 and/or wherein the CXCR2 agonist includes GRO-β.
51. The method of embodiment 49 or 50, wherein the Ad35 donor vector includes a selection cassette, optionally wherein the method further includes administering a selection agent to the subject, optionally wherein the selection cassette encodes mgmt^{P140K} and the selection agent includes O⁶BG/BCN U.
52. The method of any one of embodiments 49-51, wherein the method further includes administering to the subject an immune suppression agent, optionally wherein the immune suppression regimen includes a steroid, an IL-6 receptor antagonist, and/or an IL-1R receptor antagonist, optionally wherein the steroid includes a glucocorticoid or dexamethasone.
53. The method of any one of embodiments 49-52, wherein the Ad35 donor vector includes an integration element and the method causes integration and/or expression of a copy of the integration element thereof in at least 20%, 30%, 40%, 50%, 60%, 70%, 80%, 90%, or 95% of cells expressing CD46, in at least 20%, 30%, 40%, 50%, 60%, 70%, 80%, 90%, or 95% of hematopoietic stem cells, and/or in at least 20%, 30%, 40%, 50%, 60%, 70%, 80%, 90%, or 95% of erythroid Ter119⁺ cells.
54. The method of any one of embodiments 49-53, wherein the method causes integration of an average of at least 2 copies or at least 2.5 copies of the integration element in target cell genomes including at least 1 copy of the integration element.
55. The method of any one of embodiments 49-54, wherein the method causes expression of an expression product encoded by the payload or an integration element thereof at a level that is at least about 20% of the level of reference or at least about 25% of the level of a reference, optionally wherein the reference is expression of an endogenous reference protein in the subject or in a reference population.
56. The method of any one of embodiments 49-55, wherein the disease or condition includes a hemoglobinopathy, a platelet disorder, anemia, an immune deficiency a coagulation factor deficiency, Fanconi anemia, alpha-1 antitrypsin deficiency, sickle cell anemia, thalassemia, thalassemia intermedia, hemophilia A, hemophilia B, von Willebrand Disease, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome, or mucopolysaccharidosis.
57. The method of any one of embodiments 49-56, wherein the subject is a subject suffering from cancer and the method treats, prevents, or delays cancer, or delays cancer recurrence, optionally wherein the subject is a carrier of one or more germ-line mutation associated with development of cancer, optionally wherein the cancer includes anaplastic astrocytoma, breast cancer, ovarian cancer, colorectal cancer, diffuse intrinsic brainstem glioma, Ewing sarcoma, glioblastoma multiforme, malignant glioma, melanoma, metastatic malignant melanoma, nasopharyngeal cancer, or a pediatric cancer, optionally wherein the subject has received or is administered O⁶BG, TMZ (temozolomide), and/or BCNU (Carmustine).
58. The method of any one of embodiments 49-57, wherein the disease or condition includes thalassemia intermedia, optionally wherein the vector or genome includes a nucleic acid encoding one or more expression products selected from: expression product(s) that increase or reactivate expression of endogenous γ-globin, optionally wherein the expression product(s) that increase or reactivate expression of endogenous γ-globin includes a CRISPR-associated RNA-guided endonuclease or base editor and one or more of: a gRNA that binds a nucleic acid sequence of HBG1 and is engineered to increase expression from a coding sequence operably linked with the target nucleic acid sequence; a gRNA that binds a nucleic acid sequence of HBG2 and is engineered to increase expression from a coding sequence operably linked with the target nucleic acid sequence; and a gRNA that binds a nucleic acid sequence of erythroid enhancer bcl11a and is engineered to reduce BCL11A expression; γ-globin; and β-globin, optionally wherein the method reduces a symptom of thalassemia intermedia and/or treats thalassemia intermedia and/or increases HbF.

A second set of exemplary embodiments can include the following:

1. A recombinant serotype 35 adenovirus (Ad35) vector targeting CD46 for in vivo gene editing of hematopoietic stem cells.
2. The recombinant Ad35 vector of embodiment 1, wherein the fiber knob protein of the vector includes mutations that increase CD46 binding.
3. The recombinant Ad35 vector of embodiment 2, wherein the fiber knob protein mutations are selected from one or more of Asn217Asp, Thr254Pro, Ile256Leu, Asp207Gly (or Glu207Gly), Thr245Ala, Thr226Ala, Ile192Val, Ile256Val, Arg259Cys, and Arg279His.
4. The recombinant Ad35 vector of embodiment 2, wherein the fiber knob protein mutations include Asn217Asp, Thr254Pro, Ile256Leu, Asp207Gly (or Glu207Gly), Thr245Ala, Thr226Ala, Ile192Val, Ile256Val, Arg259Cys, and Arg279His.
5. The recombinant Ad35 vector of embodiment 2, wherein the fiber knob protein mutations consist of Asn217Asp, Thr254Pro, Ile256Leu, Asp207Gly (or Glu207Gly), Thr245Ala, Thr226Ala, Ile192Val, Ile256Val, Arg259Cys, and Arg279His.
6. The recombinant Ad35 vector of embodiment 1, including an miRNA control system that regulates expression of encoded genes in vivo.
7. The recombinant Ad35 vector of embodiment 6, wherein the miRNA control system consists of miRNA target sites with differential occupancy profiles in the blood and a tumor microenvironment or target tissue.
8. The recombinant Ad35 vector of embodiment 7, wherein the occupancy profile is higher in blood than in the tumor microenvironment or target tissue.
9. The recombinant Ad35 vector of embodiment 6, wherein the miRNA target sites include miR423-5, miR423-5p, miR42-2, miR181c, miR125a, and/or miR15a.
10. The recombinant Ad35 vector of embodiment 6, wherein the miRNA target sites control the expression of Cas9.
11. The recombinant Ad35 vector of embodiment 6, wherein the miRNA target sites include miR187, and/or miR218.
12. The recombinant Ad35 vector of embodiment 1, including nucleotides encoding CRISPR components to mediate DNA breaks and/or to activate endogenous gene expression.
13. The recombinant Ad35 vector of embodiment 12, wherein the CRISPR components include a nuclease and guide RNA.
14. The recombinant Ad35 vector of embodiment 13, wherein the nuclease includes Cas9 or cpf1.
15. The recombinant Ad35 vector of embodiment 12, wherein the CRISPR components include a catalytically disabled nuclease.
16. The recombinant Ad35 vector of embodiment 15, wherein the catalytically disabled nuclease includes a disabled Cas9 or a disabled cpf1.
17. The recombinant Ad35 vector of embodiment 15, wherein the catalytically disabled nuclease is fused to guide RNA and a cytidine or adenine deaminase or transaminase.
18. The recombinant Ad35 vector of embodiment 13, wherein the guide RNAs bind HBG1 promoter, HBG2 promoter, and/or bcl11a enhancer.
19. The recombinant Ad35 vector of embodiment 1, including a positive selection marker.
20. The recombinant Ad35 vector of embodiment 19, wherein the positive selection marker includes an anti-CD33 shRNA cassette and/or an MGMT^{P140K} cassette.
21. The recombinant Ad35 vector of embodiment 1, including homology arms.
22. The recombinant Ad35 vector of embodiment 21, wherein the homology arms are between 0.8 and 1.8 kb.
23. The recombinant Ad35 vector of embodiment 21, wherein the homology arms are specific to a chromosomal safe harbor loci.
24. The recombinant Ad35 vector of embodiment 23, wherein the chromosomal safe harbor loci is selected from AAVS1, CCR5, HPRT, or Rosa.
25. The recombinant Ad35 vector of embodiment 1, including inverted repeat sequences recognized by a transposase.
26. The recombinant Ad35 vector of embodiment 1, including a nucleotide sequence encoding a transposase.
27. The recombinant Ad35 vector of embodiment 26, wherein the transposase includes Sleeping beauty, piggyback, Mariner, frog prince, Tol2, TcBuster, and spinON.
28. The recombinant Ad35 vector of embodiment 26, wherein the transposase includes a hyperactive Sleeping beauty transposase or a hyperactive piggyBac transposase.
29. The recombinant Ad35 vector of embodiment 28, wherein the hyperactive Sleeping beauty transposase includes SB100X.
30. The recombinant Ad35 vector of embodiment 26, wherein the nucleotide sequence encoding the transposase is under the transcriptional control of a PGK promoter.
31. The recombinant Ad35 vector of embodiment 1, including recombinase recognition sequences.
32. The recombinant Ad35 vector of embodiment 31, wherein the recombinase recognition sequences include Frt, lox, rox, vox, AttB, or AttP.
33. The recombinant Ad35 vector of embodiment 1, including a nucleotide sequence encoding a recombinase.
34. The recombinant Ad35 vector of embodiment 33, wherein the recombinase includes Flp, Cre, Dre, Vika, or PhiC31.
35. The recombinant Ad35 vector of embodiment 33, wherein the nucleotide sequence encoding the recombinase is under the transcriptional control of an EF1α promoter.
36. The recombinant Ad35 vector of any one of embodiments 1-35 including a therapeutic cassette.
37. The recombinant Ad35 vector of embodiment 36, wherein the therapeutic cassette includes a therapeutic gene or encodes a therapeutic gene product selected from yC, JAK3, IL7RA, RAG1, RAG2, DCLREIC, PRKDC, LIG4, NHEJ1, CD3D, CD3ε, CD3Z, CD3G, PTPRC, ZAP70, LCK, AK2, ADA, PNP, WHN, CHD7, ORAI1, STIM1, COROIA, CIITA, RFXANK, RFX5, RFXAP, RMRP, DKC1, TERT, TINF2, DCLRE1B, SLC46A1, FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), FancW (RFWD3), soluble CD40, CTLA, Fas L, an antibody to PD-L1, an antibody to CD4, an antibody to CD5, an antibody to CD7, an antibody to CD52, an antibody to IL-1, an antibody to IL-2, an antibody to IL-4, an antibody to IL-6, an antibody to IL-10, an antibody to TNF, an antibody to a TCR specifically present on autoreactive T cells, a globin family gene, WAS, phox, dystrophin, pyruvate kinase, CLN3, ABCD1, arylsulfatase A, SFTPB, SFTPC, NLX2.1, ABCA3, GATA1, a ribosomal protein gene, TERT, TERC, DKC1, TINF2, CFTR, LRRK2, PARK2, PARK7, PINK1, SNCA, PSEN1, PSEN2, APP, SOD1, TDP43, FUS, ubiquilin 2, and/or C9ORF72.
38. The recombinant Ad35 vector of embodiment 36, wherein the therapeutic cassette includes a therapeutic gene that includes or encodes common gamma ( [symbol] ) chain, FancA, [symbol] -globin, and/or FVIII.
39. The recombinant Ad35 vector of embodiment 36, wherein the therapeutic cassette includes a therapeutic gene that encodes a chimeric antigen receptor, an engineered T cell receptor, and/or a therapeutic antibody.
40. The recombinant Ad35 vector of embodiment 38, wherein the therapeutic gene is under the transcriptional control of a β-globin promoter.
41. The recombinant Ad35 vector of embodiment 38, wherein the therapeutic gene is under the transcriptional control of a β-globin locus control region (LCR) including DNAse I hypersensitive sites (HS) consisting of HS1, HS2, HS3, and HS4.
42. The recombinant Ad35 vector of embodiment 41, wherein the β-globin LCR is approximately 4.3 kb.
43. The recombinant Ad35 vector of embodiment 41, wherein the therapeutic gene is further under the transcriptional control of a β-globin promoter.
44. The recombinant Ad35 vector of embodiment 43, wherein the β-globin promoter is approximately 1.6 kb.
45. The recombinant Ad35 vector of embodiment 44, wherein the β-globin promoter has the sequence of positions 5228631-5227023 of chromosome 11.
46. The recombinant Ad35 vector of embodiment 38, wherein the therapeutic gene is under the transcriptional control of a β-globin long LCR including HS1, HS2, HS3, HS4, and HS5.
47. The recombinant Ad35 vector of embodiment 46, wherein the β-globin long LCR is approximately 21.5 kb.
48. The recombinant Ad35 vector of embodiment 47, wherein the β-globin long LCR has the sequence of positions 5292319-5270789 of chromosome 11.
49. The recombinant Ad35 vector of embodiment 46, wherein the therapeutic gene is further under the transcriptional control of a β-globin promoter.
50. The recombinant Ad35 vector of embodiment 49, wherein the β-globin promoter is approximately 1.6 kb.
51. The recombinant Ad35 vector of embodiment 50, wherein the β-globin promoter has the sequence of positions 5228631-5227023 of chromosome 11.
52. The recombinant Ad35 vector of embodiment 46, further including a 3′HS1.
53. The recombinant Ad35 vector of embodiment 52, wherein the 3′HS1 has the sequence of positions 5206867-5203839 of chromosome 11.
54. The recombinant Ad35 vector of embodiment 1, including at least a 30 kb transposon.
55. The recombinant Ad35 vector of embodiment 1, including a 32.4 kb transposon.
56. The recombinant Ad35 vector of embodiment 1, generated using a helper virus.
57. The recombinant Ad35 vector of embodiment 56, wherein the helper virus includes the Ad5 E4orf6 for amplification in 293 T cells.
58. The recombinant Ad35 vector of embodiment 56, wherein the helper virus includes Ad35 signaling sequences and packaging sequences.
59. The recombinant Ad35 vector of embodiment 56, wherein the helper virus includes an anti-CRISPR (acr) expression cassette to prevent expression of CRISPR components during viral manufacturing.
60. The recombinant Ad35 vector of embodiment 56, wherein the helper vector includes or generates the sequence of SEQ ID NOs: 51-64.
61. An erythrocyte genetically modified to express a therapeutic protein.
62. An erythrocyte of embodiment 61, wherein the therapeutic protein includes a coagulation factor or a protein that blocks or reduces viral infection.
63. An erythrocyte of embodiment 61, wherein the erythrocyte secretes the therapeutic protein.
64. Use of a recombinant Ad35 vector or erythrocyte of any of embodiments 1-63, to increase HbF reactivation by simultaneously targeting the erythroid bcl11a-enhancer and the HBG promoter regions.
65. Use of a recombinant Ad35 vector or erythrocyte of any of embodiments 1-63, for a combination of γ-globin gene addition and endogenous γ-globin gene reactivation.
66. Use of a recombinant Ad35 vector of or erythrocyte of any of embodiments 1-63, for in vivo CRISPR genome engineering.
67. Use of a recombinant Ad35 vector or erythrocyte of any of embodiments 1-63, to provide a therapeutic gene.
68. Use of a recombinant Ad35 vector or erythrocyte of any of embodiments 1-63, to treat a (i) hemoglobinopathy, (ii) Fanconi anemia, (iii) a coagulation factor deficiency optionally selected from hemophilia A, hemophilia B, or Von Willebrand Disease, (iv) a platelet disorder, (v) anemia, (vi) alpha-1 antitrypsin deficiency, or (v) an immune deficiency.
69. Use of a recombinant Ad35 vector or erythrocyte of any of embodiments 1-63, to treat thalassemia.
70. Use of a recombinant Ad35 vector or erythrocyte of any of embodiments 1-63, to treat cancer, prevent or delay cancer recurrence or prevent or delay cancer onset in carriers of high-risk germ-line mutations, optionally wherein the cancer is breast cancer or ovarian cancer.
71. Use of a recombinant Ad35 vector or erythrocyte of any of embodiments 1-63, for self-inactivation of CRISPR/Cas9.
72. Use of a recombinant Ad35 vector or erythrocyte of any of embodiments 1-3, for targeted integration using HDAd as donor vectors with a self-releasing cassette.
73. A use of any of embodiments 64-72 including mobilization.
74. A use of embodiment 49, wherein the mobilization includes administration of Gro-beta, GM-CSF, S-CSF, and/or AMD3100.
75. A use of any of embodiments 64-72 including administering a steroid, an IL-6 receptor antagonist, and/or an IL-1R receptor antagonist to a subject receiving the Ad35 vector and/or erythrocyte.
76. The use of embodiment 75, wherein the steroid includes a glucocorticoid.
77. The use of embodiment 75, wherein the steroid includes dexamethasone.
78. A use of any of embodiments 64-72 including administering O6BG and TMZ (temozolomide) or BCNU (Carmustine) to a subject receiving the Ad35 vector and/or erythrocyte.
79. A use of embodiment 78, wherein the subject is receiving O6BG and TMZ or BCNU as a treatment for anaplastic astrocytoma, breast cancer, colorectal cancer, diffuse intrinsic brainstem glioma, Ewing sarcoma, glioblastoma multiforme (GBM), malignant glioma, melanoma, metastatic malignant melanoma, nasopharyngeal cancer, or a pediatric cancer.

A third set of exemplary embodiments can include the following:

1. A recombinant serotype 35 adenovirus (Ad35) vector targeting CD46 for in vivo gene editing of hematopoietic stem cells.
2. The recombinant Ad35 vector of embodiment 1, wherein the fiber knob protein of the vector includes mutations that increase CD46 binding.
3. The recombinant Ad35 vector of embodiment 2, wherein the fiber knob protein mutations are selected from one or more of Asn217Asp, Thr254Pro, Ile256Leu, Asp207Gly (or Glu207Gly), Thr245Ala, Thr226Ala, Ile192Val, Ile256Val, Arg259Cys, and Arg279His.
4. The recombinant Ad35 vector of embodiment 2, wherein the fiber knob protein mutations include Asn217Asp, Thr254Pro, Ile256Leu, Asp207Gly (or Glu207Gly), Thr245Ala, Thr226Ala, Ile192Val, Ile256Val, Arg259Cys, and Arg279His.
5. The recombinant Ad35 vector of embodiment 2, wherein the fiber knob protein mutations consist of Asn217Asp, Thr254Pro, Ile256Leu, Asp207Gly (or Glu207Gly), Thr245Ala, Thr226Ala, Ile192Val, Ile256Val, Arg259Cys, and Arg279His.
6. The recombinant Ad35 vector of embodiment 1, including an miRNA control system that regulates expression of encoded genes in vivo.
7. The recombinant Ad35 vector of embodiment 6, wherein the miRNA control system consists of miRNA target sites with differential occupancy profiles in the blood and a tumor microenvironment or target tissue.
8. The recombinant Ad35 vector of embodiment 7, wherein the occupancy profile is higher in blood than in the tumor microenvironment or target tissue.
9. The recombinant Ad35 vector of embodiment 6, wherein the miRNA target sites include miR423-5, miR423-5p, miR42-2, miR181c, miR125a, and/or miR15a.
10. The recombinant Ad35 vector of embodiment 6, wherein the miRNA target sites control the expression of Cas9.
11. The recombinant Ad35 vector of embodiment 6, wherein the miRNA target sites include miR187, and/or miR218.
12. The recombinant Ad35 vector of embodiment 1, including nucleotides encoding CRISPR components to mediate DNA breaks and/or to activate endogenous gene expression.
13. The recombinant Ad35 vector of embodiment 12, wherein the CRISPR components include a nuclease and guide RNA.
14. The recombinant Ad35 vector of embodiment 13, wherein the nuclease includes Cas9 or cpf1.
15. The recombinant Ad35 vector of embodiment 12, wherein the CRISPR components include a catalytically disabled nuclease.
16. The recombinant Ad35 vector of embodiment 15, wherein the catalytically disabled nuclease includes a disabled Cas9 or a disabled cpf1.
17. The recombinant Ad35 vector of embodiment 15, wherein the catalytically disabled nuclease is fused to guide RNA and a cytidine or adenine deaminase or transaminase.
18. The recombinant Ad35 vector of embodiment 13, wherein the guide RNAs bind HBG1, HBG2, and/or Bc11a.
19. The recombinant Ad35 vector of embodiment 1, including a positive selection marker.
20. The recombinant Ad35 vector of embodiment 19, wherein the positive selection marker includes an anti-CD33 shRNA cassette and/or an MGMTPl⁴Ok cassette.
21. The recombinant Ad35 vector of embodiment 1, including homology arms.
22. The recombinant Ad35 vector of embodiment 21, wherein the homology arms are between 0.8 and 1.8 kb.
23. The recombinant Ad35 vector of embodiment 21, wherein the homology arms are specific to a chromosomal safe harbor loci.
24. The recombinant Ad35 vector of embodiment 23, wherein the chromosomal safe harbor loci is selected from AAVS1, CCR5, HPRT, or Rosa.
25. The recombinant Ad35 vector of embodiment 1, including inverted repeat sequences recognized by a transposase.
26. The recombinant Ad35 vector of embodiment 1, including a nucleotide sequence encoding a transposase.
27. The recombinant Ad35 vector of embodiment 26, wherein the transposase includes Sleeping beauty, piggyback, Mariner, frog prince, Tol2, TcBuster, and spinON.
28. The recombinant Ad35 vector of embodiment 26, wherein the transposase includes a hyperactive Sleeping beauty transposase or a hyperactive piggybac transposase.
29. The recombinant Ad35 vector of embodiment 28, wherein the hyperactive Sleeping beauty transposase includes SB100X.
30. The recombinant Ad35 vector of embodiment 26, wherein the nucleotide sequence encoding the transposase is under the transcriptional control of a PGK promoter.
31. The recombinant Ad35 vector of embodiment 1, including recombinase recognition sequences.
32. The recombinant Ad35 vector of embodiment 31, wherein the recombinase recognition sequences include Frt, lox, rox, vox, AttB, or AttP.
33. The recombinant Ad35 vector of embodiment 1, including a nucleotide sequence encoding a recombinase.
34. The recombinant Ad35 vector of embodiment 33, wherein the recombinase includes Flp, Cre, Dre, Vika, or PhiC31.
35. The recombinant Ad35 vector of embodiment 33, wherein the nucleotide sequence encoding the recombinase is under the transcriptional control of an EF1α promoter.
36. The recombinant Ad35 vector of any one of embodiments 1-35 including a therapeutic cassette.
37. The recombinant Ad35 vector of embodiment 36, wherein the therapeutic cassette includes a therapeutic gene or encodes a therapeutic gene product selected from γC, JAK3, IL7RA, RAG1, RAG2, DCLREIC, PRKDC, LIG4, NHEJ1, CD3D, CD3ε, CD3Z, CD3G, PTPRC, ZAP70, LCK, AK2, ADA, PNP, WHN, CHD7, ORAI1, STIM1, COROIA, CIITA, RFXANK, RFX5, RFXAP, RMRP, DKC1, TERT, TINF2, DCLRE1B, SLC46A1, FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), FancW (RFWD3), soluble CD40, CTLA, Fas L, an antibody to PD-L1, an antibody to CD4, an antibody to CD5, an antibody to CD7, an antibody to CD52, an antibody to IL-1, an antibody to IL-2, an antibody to IL-4, an antibody to IL-6, an antibody to IL-10, an antibody to TNF, an antibody to a TCR specifically present on autoreactive T cells, a globin family gene, WAS, phox, dystrophin, pyruvate kinase, CLN3, ABCD1, arylsulfatase A, SFTPB, SFTPC, NLX2.1, ABCA3, GATA1, a ribosomal protein gene, TERT, TERC, DKC1, TINF2, CFTR, LRRK2, PARK2, PARK7, PINK1, SNCA, PSEN1, PSEN2, APP, SOD1, TDP43, FUS, ubiquilin 2, and/or C9ORF72.
38. The recombinant Ad35 vector of embodiment 36, wherein the therapeutic cassette includes a therapeutic gene that includes or encodes common gamma (γ) chain, FancA, γ-globin, and/or FVIII.
39. The recombinant Ad35 vector of embodiment 36, wherein the therapeutic cassette includes a therapeutic gene that encodes a chimeric antigen receptor, an engineered T cell receptor, and/or a therapeutic antibody.
40. The recombinant Ad35 vector of embodiment 38, wherein the therapeutic gene is under the transcriptional control of a β-globin promoter.
41. The recombinant Ad35 vector of embodiment 38, wherein the therapeutic gene is under the transcriptional control of a β-globin locus control region (LCR) including DNAse I hypersensitive sites (HS) consisting of HS1, HS2, HS3, and HS4.
42. The recombinant Ad35 vector of embodiment 41, wherein the β-globin LCR is approximately 4.3 kb.
43. The recombinant Ad35 vector of embodiment 41, wherein the therapeutic gene is further under the transcriptional control of a β-globin promoter.
44. The recombinant Ad35 vector of embodiment 43, wherein the β-globin promoter is approximately 1.6 kb.
45. The recombinant Ad35 vector of embodiment 44, wherein the β-globin promoter has the sequence of positions 5228631-5227023 of chromosome 11.
46. The recombinant Ad35 vector of embodiment 38, wherein the therapeutic gene is under the transcriptional control of a β-globin long LCR including HS1, HS2, HS3, HS4, and HS5.
47. The recombinant Ad35 vector of embodiment 46, wherein the β-globin long LCR is approximately 21.5 kb.
48. The recombinant Ad35 vector of embodiment 47, wherein the β-globin long LCR has the sequence of positions 5292319-5270789 of chromosome 11.
49. The recombinant Ad35 vector of embodiment 46, wherein the therapeutic gene is further under the transcriptional control of a β-globin promoter.
50. The recombinant Ad35 vector of embodiment 49, wherein the β-globin promoter is approximately 1.6 kb.
51. The recombinant Ad35 vector of embodiment 50, wherein the β-globin promoter has the sequence of positions 5228631-5227023 of chromosome 11.
52. The recombinant Ad35 vector of embodiment 46, further including a 3′HS1.
53. The recombinant Ad35 vector of embodiment 52, wherein the 3′HS1 has the sequence of positions 5206867-5203839 of chromosome 11.
54. The recombinant Ad35 vector of embodiment 1, including at least a 30 kb transposon.
55. The recombinant Ad35 vector of embodiment 1, including a 32.4 kb transposon.
56. The recombinant Ad35 vector of embodiment 1, generated using a helper virus.
57. The recombinant Ad35 vector of embodiment 56, wherein the helper virus includes the Ad5 E4orf6 for amplification in 293 T cells.
58. The recombinant Ad35 vector of embodiment 56, wherein the helper virus includes Ad35 signaling sequences and packaging signals.
59. The recombinant Ad35 vector of embodiment 56, wherein the helper virus includes an anti-CRISPR (acr) expression cassette to prevent expression of CRISPR components during viral manufacturing.
60. The recombinant Ad35 vector of embodiment 56, wherein the helper vector includes or generates the sequence of any one of SEQ ID NOs: 51-65.
61. An erythrocyte genetically modified to express a therapeutic protein.
62. An erythrocyte of embodiment 61, wherein the therapeutic protein includes a coagulation factor or a protein that blocks or reduces viral infection.
63. An erythrocyte of embodiment 61, wherein the erythrocyte secretes the therapeutic protein.
64. Use of a recombinant Ad35 vector or erythrocyte of any of embodiments 1-63, to increase HbF reactivation by simultaneously targeting the erythroid bcl11a-enhancer and the HBG promoter regions.
65. Use of a recombinant Ad35 vector or erythrocyte of any of embodiments 1-63, for a combination of γ-globin gene addition and endogenous γ-globin gene reactivation.
66. Use of a recombinant Ad35 vector of or erythrocyte of any of embodiments 1-63, for in vivo CRISPR genome engineering.
67. Use of a recombinant Ad35 vector or erythrocyte of any of embodiments 1-63, to provide a therapeutic gene.
68. Use of a recombinant Ad35 vector or erythrocyte of any of embodiments 1-63, to treat a (i) hemoglobinopathy, (ii) Fanconi anemia, (iii) a coagulation factor deficiency optionally selected from hemophilia A, hemophilia B, or Von Willebrand Disease, (iv) a platelet disorder, (v) anemia, (vi) alpha-1 antitrypsin deficiency, or (v) an immune deficiency.
69. Use of a recombinant Ad35 vector or erythrocyte of any of embodiments 1-63, to treat thalassemia.
70. Use of a recombinant Ad35 vector or erythrocyte of any of embodiments 1-63, to treat cancer, prevent or delay cancer recurrence or prevent or delay cancer onset in carriers of high-risk germ-line mutations, optionally wherein the cancer is breast cancer or ovarian cancer.
71. Use of a recombinant Ad35 vector or erythrocyte of any of embodiments 1-63, for self-inactivation of CRISPR/Cas9.
72. Use of a recombinant Ad35 vector or erythrocyte of any of embodiments 1-3, for targeted integration using HDAd as donor vectors with a self-releasing cassette.
73. A use of any of embodiments 64-72 including mobilization.
74. A use of embodiment 49, wherein the mobilization includes administration of Gro-beta, GM-CSF, S-CSF, and/or AMD3100.
75. A use of any of embodiments 64-72 including administering a steroid, an IL-6 receptor antagonist, and/or an IL-1R receptor antagonist to a subject receiving the Ad35 vector and/or erythrocyte.
76. The use of embodiment 75, wherein the steroid includes a glucocorticoid.
77. The use of embodiment 75, wherein the steroid includes dexamethasone.
78. A use of any of embodiments 64-72 including administering O⁶BG and TMZ (temozolomide) or BCNU (Carmustine) to a subject receiving the Ad35 vector and/or erythrocyte.
79. A use of embodiment 78, wherein the subject is receiving O⁶BG and TMZ or BCNU as a treatment for anaplastic astrocytoma, breast cancer, colorectal cancer, diffuse intrinsic brainstem glioma, Ewing sarcoma, glioblastoma multiforme (GBM), malignant glioma, melanoma, metastatic malignant melanoma, nasopharyngeal cancer, or a pediatric cancer.

### VI. EXPERIMENTAL EXAMPLES

### Example 1. In Vivo Hematopoietic Stem Cell Gene Therapy Ameliorates Murine Thalassemia Intermedia

This example illustrates an in vivo HSPC gene therapy approach employing an integrating HDAd5/35++ vector expressing the human γ-globin gene in “healthy” human CD46-transgenic (CD46tg) mice; as a proof of concept, this approach is illustrated in a mouse model for Thalassemia intermedia (CD46^{+/+}/Hbbth-3 mice). This provides an alternative to traditional lentivirus vector ex vivo gene therapy for thalassemia. At least some of the information contained in this example was published in Wang et al., (J Clin Invest. 129(2):598-615, 2019; e-pub Nov. 13, 2018).

Thalassemia is one of the most common inherited diseases in humans worldwide (Weatherall, Ann N Y Acad Sci. 1202:17-23, 2010), resulting from absent (β0/β0) or deficient (β+/β+) β-globin chain synthesis. 60,000 children are born annually with β-thalassemia major. Without treatment, children with thalassemia major die in their first to second decade of life. In the absence of sufficient β-globin chain synthesis for hemoglobin tetramer formation, excess α-globin chains precipitate and form inclusions that cause the premature death of late erythroblasts in the bone marrow or reduce the half-life of circulating erythrocytes, generating the major hematological hallmarks of β-thalassemia, ineffective erythropoiesis and erythrocyte death. The resulting anemia stimulates the expansion of the hematopoietic compartment, producing erythroid hyperplasia and extramedullary hematopoiesis.

The major treatment modalities for β-thalassemia major consist of supportive care with lifelong transfusions of red blood cells (RBCs) and chelation to remove excess iron; or curative treatment with transplantation of allogeneic hematopoietic stem/progenitor cells (HSPCs). For patients lacking a well-matched donor or at risk to undergo an allogeneic HSPC transplantation, lentiviral vector wild-type β-globin or fetal γ-globin gene therapy has the potential for a cure bypassing the immunological risks of allogeneic transplantation. HSPC gene therapy with SIN-lentiviral globin vectors, incorporating micro-LCR cassettes, rescued β-thalassemia and sickle cell disease (SCD) phenotypes in animal models and in patient cells in vitro (Pstaha et al., Curr Gene Ther. 17(5):364-378, 2017). Based on this, a number of clinical trials for thalassemia and SCD are currently ongoing in Europe, Asia, and the United States (Pstaha et al., Curr Gene Ther. 17(5):364-378, 2017, Cavazanna-Calvo et al., Nature. 467(7313):318-322, 2010, Ferarri et al., Hematology/Oncology Clinics of North America: Gene Therapy. 31(5), Thompson et al., N Engl J Med. 378(16):1479-1493, 2018). While the data from these trials so far demonstrate long-term transfusion independence for the majority of patients having a β+ genotype, the cure of β0/β0 thalassemia still remains a challenge.

Despite the encouraging clinical results, current thalassemia gene therapy protocols are complex, involving the collection of HSPCs from donors/patients by leukapheresis, in vitro culture, transduction with lentivirus vectors carrying a β- or γ-globin expression cassette, and retransplantation into patients conditioned with full myeloablation. Besides the technical complexity, other disadvantages of this approach include (a) the necessity for culture in the presence of multiple cytokines, which can affect the pluripotency of hematopoietic stem cells (HSCs) and their engraftment potential; (b) the requirement for myeloablative regimens, as myeloablation in patients with chronic, nonmalignant diseases and preexisting organ damage, such as those with hemoglobinopathies, represents a critical risk factor associated with considerable hematopoietic and nonhematopoietic, early or late toxicity; and (c) the cost of the approach. The fact that thalassemia is prevalent in resource-poor countries demands a simpler and cheaper therapy approach.

A minimally invasive and readily translatable approach for in vivo HSPC gene delivery without leukapheresis, myeloablation, and HSPC transplantation has been developed (Richter et al., Blood. 2016; 128(18):2206-2217, Richter et al., Hematol Oncol Clin North Am. 31(5):771-785, 2017, Ren et al., Blood. 128(18):2194-219, 2016). It involves injections of G-CSF/AMD3100 to mobilize HSPCs from the bone marrow into the peripheral bloodstream and the intravenous injection of an integrating, helper-dependent adenovirus (HDAd5/35++) vector system. HDAd5/35++ vectors target human CD46, a receptor that is expressed on primitive HSCs (Richter et al., Blood. 128(18):2206-2217, 2016). In HDAd5/35++, all proteins except the fiber knob domain and shaft are derived from serotype 5; the fiber knob domain and shaft are derived from serotype 35; mutations that increase the affinity to CD46 are introduced into the Ad35 fiber knob (see WO 2010/0120541) and the ITR and packaging signal are derived from Ad5. In HdAd35++, all proteins are derived from serotype 35; mutations that increase the affinity to CD46 are introduced into the fiber knob and the ITR and packaging signal are derived from Ad35.

Transgene integration is achieved, in a random pattern, by a hyperactive Sleeping Beauty transposase (SB100X) (Mátés et al., Nat Genet. 41(6):753-761, 2009). It was demonstrated in mouse models, using GFP as a reporter gene, that HSPCs transduced in the periphery home back to the bone marrow, where they persist and stably express the reporter long-term in in vivo-transduced mice and secondary recipients (Richter et al., Blood. 2016; 128(18):2206-2217).

Given the high level of transgene marking required in order to phenotypically correct thalassemia, the in vivo HSPC transduction approach was optimized by inserting an MGMT^{P140K} expression cassette into HDAd5/35++ vectors. This allows for in vivo selection of gene-corrected progenitors with low doses of methylating agents, e.g., O⁶-benzylguanine (O⁶BG) plus bis-chloroethylnitrosourea (BCNU) or temozolomide (Beard et al., J Clin Invest. 120(7):2345-2354, 2010, Larochelle et al., J Clin Invest. 119(7):1952-1963, 2009, Trobridge et al., PLoS One. 7(9):e45173, 2012). It was previously shown that the combined in vivo transduction/selection approach was safe and resulted in stable GFP expression in up to 80% of peripheral blood cells, a level that was maintained in secondary recipients, indicating the stable transduction of self-renewing, multilineage, long-term repopulating HSCs (Wang et al., Mol Ther Methods Clin Dev. 8:52-64, 2018).

Herein the in vivo HSPC gene therapy approach was tested using an integrating HDAd5/35++vector expressing the human γ-globin gene in “healthy” human CD46-transgenic (CD46tg) mice and, as a proof of concept, in a mouse model for thalassemia intermedia (CD46^{+/+}/Hbbth-3 mice).

Materials and Methods. Reagents. The following reagents were used: G-CSF (Neupogen, Amgen), AMD3100 (Sigma-Aldrich), plerixafor (Mozobil, Genzyme Corp.), O⁶-BG and BCNU (Sigma-Aldrich), mycophenolate mofetil (CellCept Intravenous, Genentech), rapamycin (Rapamune/Sirolimus, Pfizer), and methylprednisolone (Pfizer).

HDAd vectors. The generation of the transposon vector HDAd-γ-globin/mgmt and the SB100X-expressing human embryonic kidney-293 cell-derived 116 cells (Palmer et al., Gene Therapy Protocols. Volume 1: Production and In vivo Applications of Gene Transfer Vectors (Methods in Molecular Biology):33-53, 2009) has been described previously (Li et al., Mol Ther Methods Clin Dev. 9:142-152, 2018). Helper virus contamination levels were found to be less than 0.05%. Titers were 6×10¹² to 12×10¹² vp/ml. All HDAd vectors used in this study contain chimeric fibers composed of the Ad5 fiber tail, the Ad35 fiber shaft, and the affinity-enhanced Ad35++fiber knob (Wang et al., J Virol. 82(21):10567-10579, 2008). All of the HDAd preparations had less than 1 copy wild-type virus in 10¹⁰ vp (measured by qPCR using the primers described elsewhere; Haeussler et al., PLoS One. 6(8):e23160, 2011).

Intracellular flow cytometry detecting human γ-globin expression. The FIX and PERM cell permeabilization kit (Thermo Fisher Scientific) was used, and the manufacturer's protocol was followed. Briefly, 1×10⁶ cells were resuspended in 100 μl FACS buffer (PBS supplemented with 1% FCS), 100 μl reagent A (fixation medium) was added and incubated for 2-3 minutes at room temperature, and 1 ml precooled absolute methanol was then added, mixed, and incubated on ice in the dark for 10 minutes. The samples were then washed with FACS buffer and resuspended in 100 μl reagent B (permeabilization medium) and 1 μg γ-globin antibody (Santa Cruz Biotechnology, catalog sc-21756 PE), incubated for 30 minutes at room temperature. After the wash, cells were resuspended in FACS buffer and analyzed. For erythroid and γ-globin double staining, cells were stained with APC anti-mouse Ter119 antibody (Ter119-APC, BioLegend, catalog 116212) first, and then washed and fixed with fixation medium as described above.

Globin HPLC. Individual globin chain levels were quantified on a Shimadzu Prominence instrument with an SPD-10AV diode array detector and an LC-10AT binary pump (Shimadzu). A 38%-60% gradient mixture of 0.1% trifluoroacetic acid in water/acetonitrile was applied at a rate of 1 ml/min using a Vydac C4 reversed-phase column (Hichrom).

Real-time reverse transcription PCR. Total RNA was extracted from 50-100 μl blood using TRIzol™ reagent (Thermo Fisher Scientific, Cat. #15596026) following the manufacturer's phenol-chloroform extraction method. QuantiTect reverse transcription kit (Qiagen, Cat. #205311) and Power SYBR Green PCR Master Mix (Thermo Fisher Scientific, Cat. #4367659) were used. Real-time quantitative PCR was performed on a StepOnePlus Real-Time PCR System (Applied Biosystems). The following primer pairs were used in this work: mouse RPL10 forward (SEQ ID NO: 189), and reverse (SEQ ID NO: 190); human γ-globin forward (SEQ ID NO: 191), and reverse (SEQ ID NO: 192); mouse β-major globin forward (SEQ ID NO: 193), and reverse (SEQ ID NO: 194).

Magnetic cell sorting. For the depletion of lineage-committed cells, the mouse Lineage Cell Depletion Kit (Miltenyi Biotec, Cat. #130-090-858) was used according to the manufacturer's instructions. For the selection of Ter119⁺ cells from the bone marrow of primary CD46^{+/+}/Hbbth-3 mice or CD46+ cells from the hematopoietic tissues of secondary C57BL/6 recipients, mouse anti-Ter119 microbeads (Miltenyi Biotec, catalog 130-049-901) or anti-PE microbeads (Miltenyi Biotec, catalog 130-048-801) following staining with human anti-CD46-PE primary antibody (Miltenyi Biotec, catalog 130-104-508) were used, respectively.

Animal studies. C57BL/6-based transgenic mice that are homozygous for the human CD46 genomic locus (CD46tg) and provide CD46 expression at a level and in a pattern similar to that in humans were described earlier (Kemper et al., Clin Exp Immunol. 2001; 124(2):180-189). CD46tg mice were provided by Roberto Cattaneo, Mayo Clinic (Rochester, Minn., USA). A thalassemic mouse model susceptible to infection by HDAd5/35++ vectors was obtained by breeding of female CD46tg mice with male Hbbth-3 mice (The Jackson Laboratory) and backcrossing of F1 with CD46tg mice, to generate CD46^{+/+}/Hbbth-3 mice. Six- to ten-week-old female CD46tg and CD46^{+/+}/Hbbth-3 females were used for the in vivo transduction/selection studies. Six- to ten-week-old female C57BL/6 mice were used as secondary recipients.

Mobilization and in vivo transduction of CD46tg mice. HSPCs were mobilized in mice by s.c. injections of human recombinant G-CSF (5 μg/mouse per day, 4 days) followed by an s.c. injection of AMD3100 (5 mg/kg) on day 5. In addition, animals received dexamethasone (10 mg/kg) i.p. 16 and 2 hours before virus injection. Thirty and 60 minutes after AMD3100, animals were injected i.v. with HDAd-γ-globin/mgmt plus HDAd-SB through the retro-orbital plexus with a dose of 4×10¹⁰ vp per injection (total 2 injections, 30 minutes apart). Four weeks later, mice were injected with O⁶-BG (15 mg/kg, i.p.) 2 times, 30 minutes apart. One hour after the second injection of O⁶-BG, mice were injected with BCNU (5 mg/kg, i.p.). The BCNU dose was increased in the second and third cycles to 7.5 and 10 mg/kg, respectively.

Mobilization and in vivo transduction of CD46^{+/+}/Hbbth-3 mice. In these studies, a 7-day mobilization approach with G-CSF 250 μg/kg i.p. (1-6 days) and plerixafor 5 mg/kg i.p. (formerly AMD3100; Mozobil, Genzyme Corp.) (days 5-7) was applied, as previously described in a thalassemic mouse model (Psatha et al., Hum Gene Ther Methods. 25(6):317-327, 2014). In vivo transduction was performed as above. Following treatment, combined immunosuppression was administered. At week 17, mice were subjected to 4 cycles of in vivo selection with O⁶BG (30 mg/kg, i.p.) and escalated BCNU doses (5, 7.5, 10, 10 mg/kg) with a 2-week interval between doses. Immunosuppression was resumed 2 weeks after the last O⁶-BG/BCNU dose.

Immunosuppression. Daily i.p. injection of mycophenolate mofetil (20 mg/kg/d), rapamycin (0.2 mg/kg/d), and methylprednisolone (20 mg/kg/d) was performed.

Secondary bone marrow transplantation. Recipients were female C57BL/6 mice, 6-8 weeks old, from The Jackson Laboratory. On the day of transplantation, recipient mice were irradiated with 10 Gy. Bone marrow cells from in vivo-transduced CD46tg mice were isolated aseptically, and lineage-depleted cells were isolated using magnetic cell sorting (MACS). Four hours after irradiation, cells were injected i.v. at 1×10⁶ cells per mouse. In the CD46^{+/+}/Hbbth-3 mouse studies, 2×10⁶ whole bone marrow cells from in vivo-transduced CD46^{+/+}/Hbbth-3 mice were transplanted into submyeloablated secondary C57BL/6 recipients conditioned with 100 mg/kg i.p. busulfan (Busilvex, Pierre Fabre) divided into 3 daily doses or lethal TBI (1,000 cGy). At week 20, secondary recipients were sacrificed, and CD46+ cells from blood, bone marrow, and spleen were either isolated by MACS, or mice were subjected to mobilization and in vivo transduction, as described above. All secondary recipients received immunosuppression starting at week 4.

Tissue analysis. Spleen and liver tissue sections of 2.5 μm thickness were fixed in 4% formaldehyde for at least 24 hours, dehydrated, and embedded in paraffin. Staining with H&E was used for histological evaluation of extramedullary hemopoiesis. Hemosiderin was detected in tissue sections by Perls' Prussian blue staining. Briefly, the tissue sections were treated with a mixture of equal volumes (2%) of potassium ferrocyanide and hydrochloric acid in distilled water and then counterstained with neutral red. The spleen size was assessed as the ratio of spleen weight (mg) to body weight (g).

Blood analysis and bone marrow cytospins. Blood samples were collected into EDTA-coated tubes, and analysis was performed on a HemaVet 950FS (Drew Scientific) or ProCyteDx (IDEXX) machine. Peripheral blood smears were prepared and stained with May-Grunwald/Giemsa for 5 and 15 minutes, respectively (Merck). Suspensions of bone marrow cells were centrifuged onto slides using a cytospin device and stained with May-Grunwald/Giemsa.

Statistics. Data are presented as means±SEM. For comparisons of multiple groups, 1-way and 2-way ANOVA with Bonferroni post-testing for multiple comparisons was used. Differences between groups for 1 grouping variable were determined by unpaired, 2-tailed Student's t test. For nonparametric analyses, the Kruskal-Wallis test was used. Statistical analysis was performed using GraphPad Prism version 6.01 (GraphPad Software Inc.). A P value less than 0.05 was considered significant; *P≤0.05, **P≤0.0002, ***P≤0.00003.

Animal study approval. All experiments were conducted with approval from the controlling Institutional Review Board and IACUC.

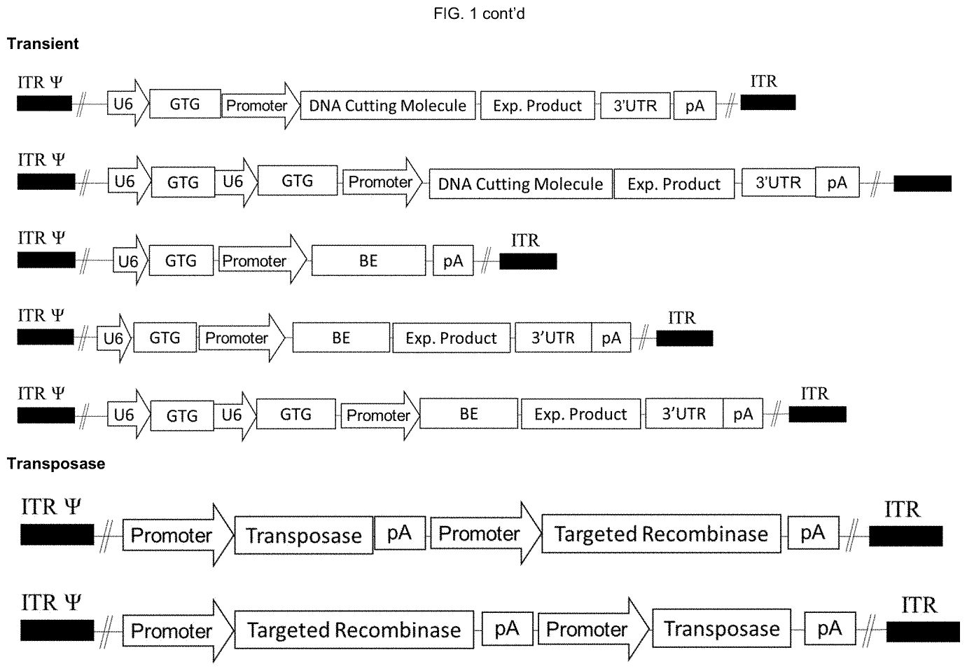

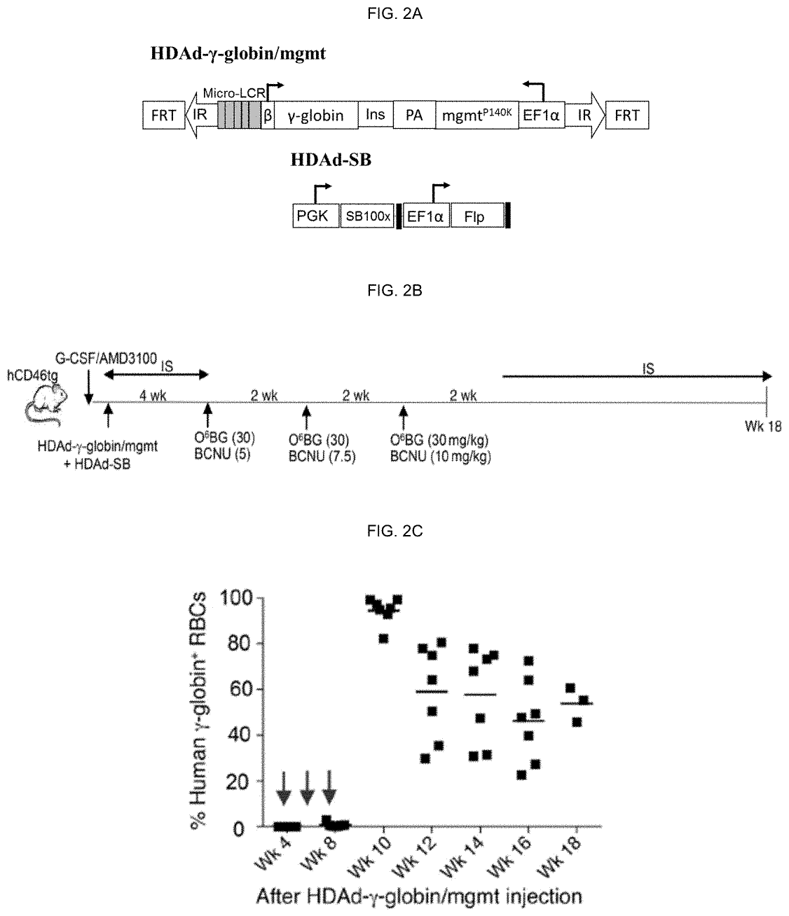

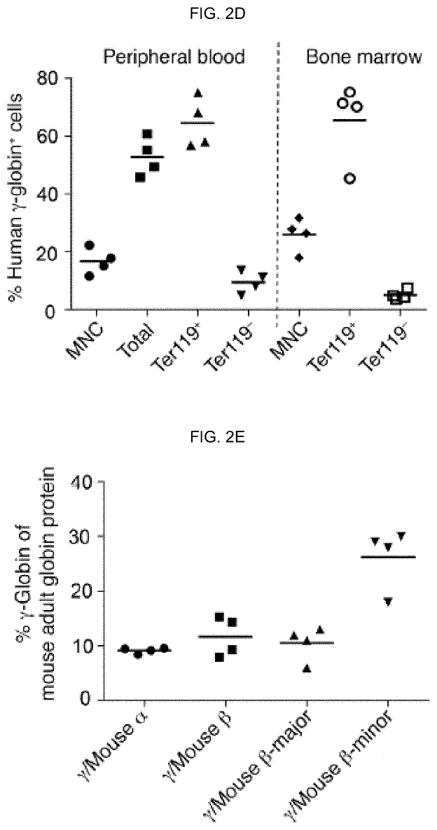

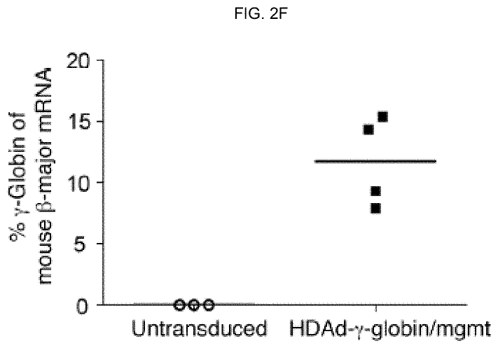

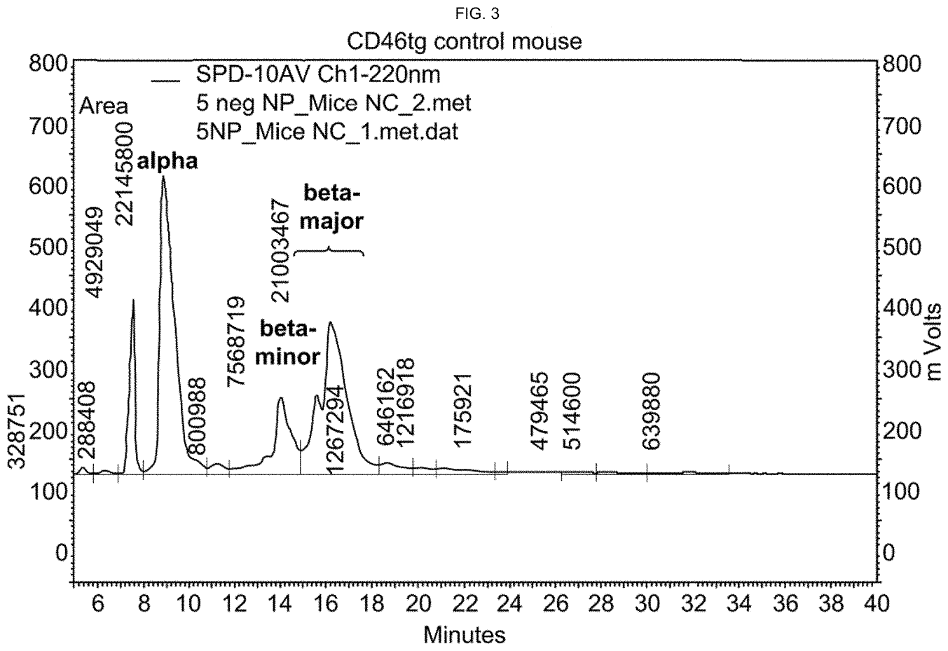

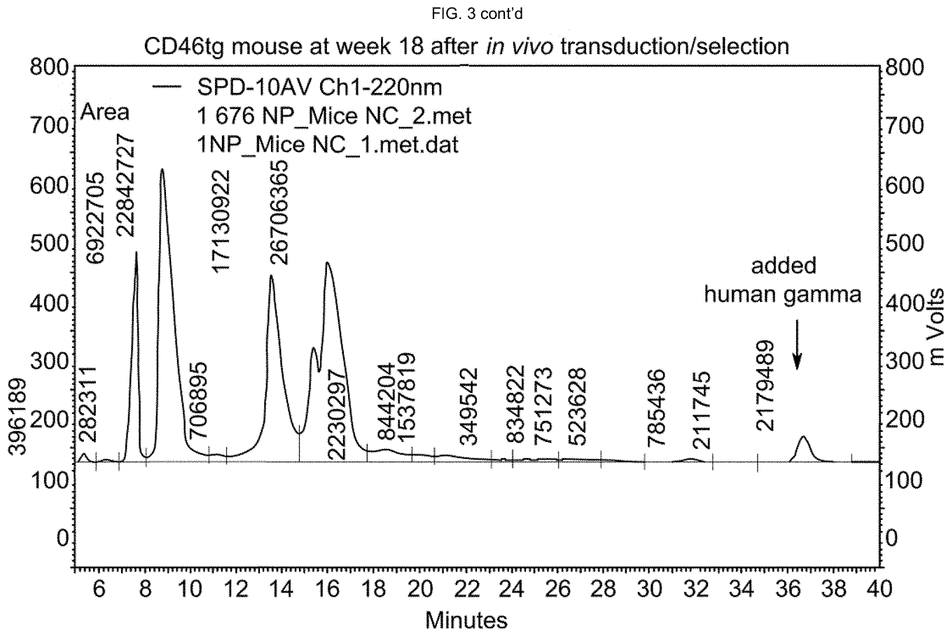

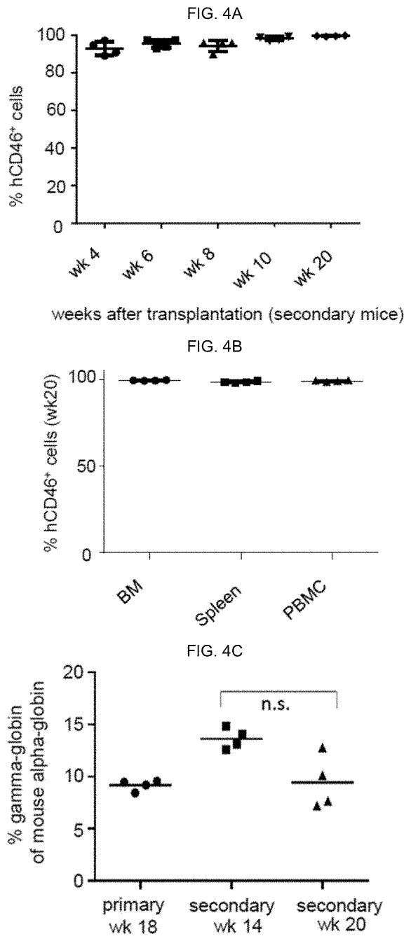

Results. In vivo HSPC transduction with subsequent in vivo selection in CD46tg mice results in stable γ-globin expression in the majority of peripheral RBCs. The therapeutic HDAd5/35++ vector contains the human γ-globin gene under the control of 5-kb “micro” β-globin LCR/β-promoter for efficient expression in erythrocytes as well as an MGMT^{P140K} expression cassette (FIG. 2A, HDAd-γ-globin/mgmt). CD46tg mice are homozygous for the human CD46 locus expressing the HDAd5/35++receptor CD46 in a pattern and at a level similar to that in humans and are therefore a model for in vivo HSPC transduction studies (Richter et al., Blood. 128(18):2206-2217, 2016, Kemper et al., Clin Exp Immunol. 124(2):180-189, 2001). The goal of these studies in “healthy” CD46tg mice was to analyze the level, kinetics, and distribution of human γ-globin on mouse cells and the safety of the approach. Animals were mobilized with G-CSF/AMD3100 and then intravenously injected with HDAd-γ-globin/mgmt and the SB100X-expressing HDAd-SB vector. Three cycles of O⁶BG/BCNU treatment were initiated 4 weeks after vector injection, and mice were followed until week 18 after injection of the vectors (FIG. 2B). First, human γ-globin expression in RBCs was analyzed (FIG. 2C). The levels before the start of in vivo selection (week 4 after transduction) were only marginally above background. The percentage of γ-globin⁺ cells started to increase after the second round of selection and reached levels above 80% after the third round. The percentage of γ-globin-expressing cells was 7- to 10-fold higher in erythroid Ter119⁺ cells versus nonerythroid Ter119-cells in peripheral blood and bone marrow (FIG. 2D). HPLC was used to measure the level of γ-globin protein in comparison with the adult mouse α- and β-globin chains (FIG. 2E and FIG. 3; supplemental material at https://doi.org/10.1172/JCI122836DS1). At week 18, these levels reached 10%-15% of adult mouse α-globin and β-major globin and 25% of mouse β-minor globin. This was confirmed on the mRNA level by quantitative reverse transcription PCR (RT-qPCR), where human γ-globin mRNA was 13% of mouse β-major mRNA (FIG. 2F). To further demonstrate that primitive, long-term repopulating HSCs were transduced, lineage-depleted (Lin−) bone marrow cells from in vivo-transduced/selected mice were transplanted into irradiated C57BL/6 mice. Engraftment levels analyzed in peripheral blood, bone marrow, and spleen were greater than 95% and stable over an observation period of 20 weeks (FIGS. 4A, 4B). Human γ-globin levels (compared with mouse α-globin) were similar in (“primary”) in vivo-transduced mice (analyzed at week 18 after transduction) and secondary recipients analyzed at weeks 14 and 20 after transplantation (FIG. 4C).

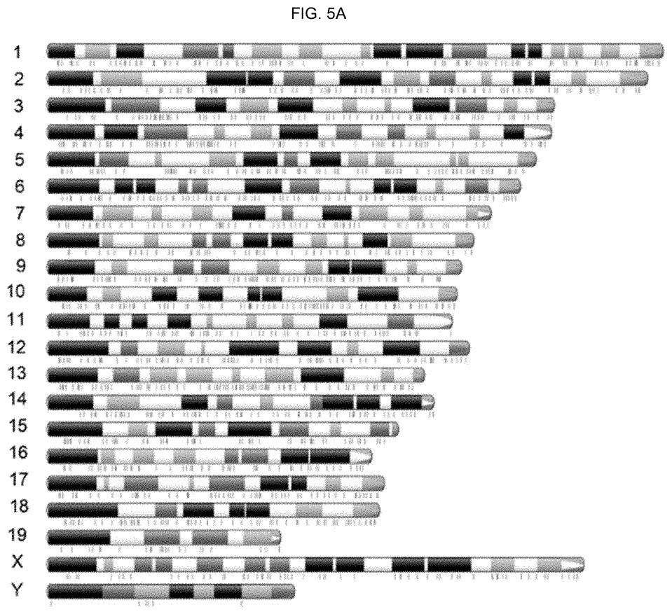

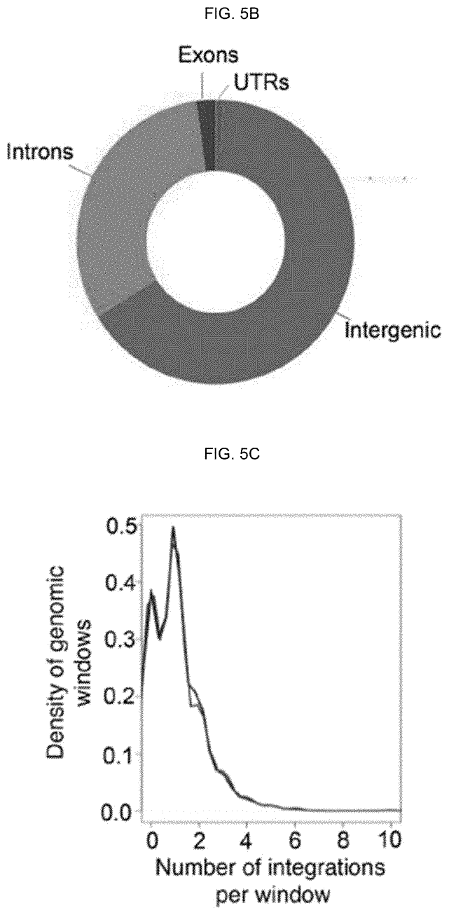

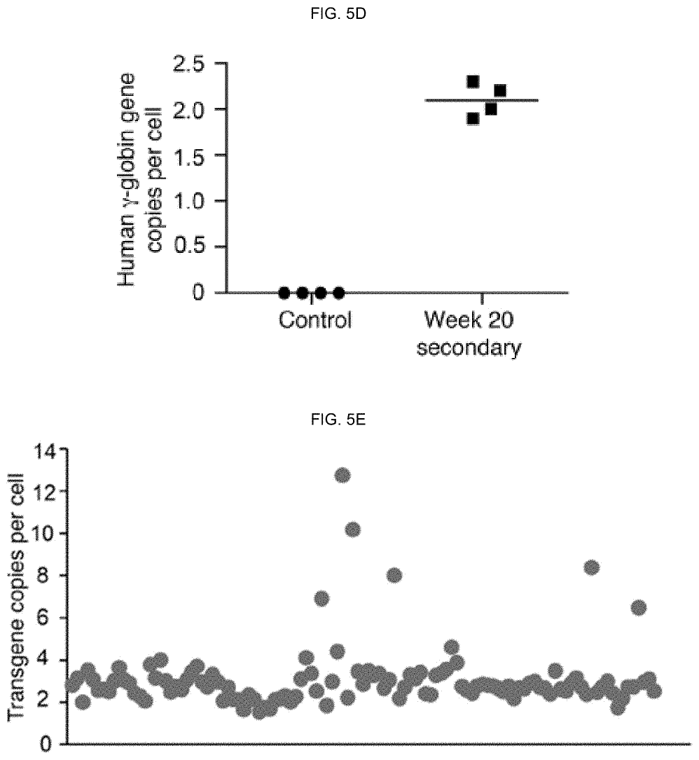

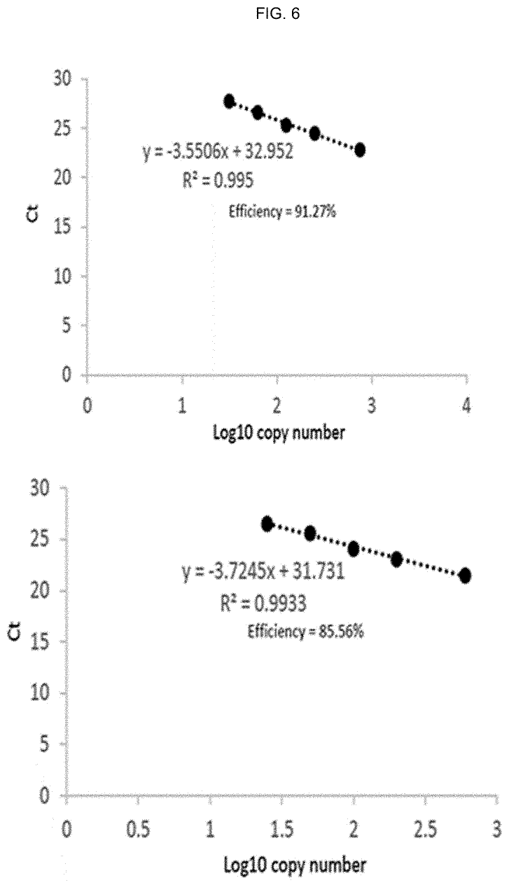

The in vivo HSPC transduction/selection approach does not change the SB100X-mediated random transgene integration pattern and does not alter hematopoiesis. It was previously shown that in vivo transduction with the hybrid transposon/SB100X HDAd5/35++system resulted in random transgene integration in HSPCs (Richter et al., Blood. 128(18):2206-2217, 2016). To evaluate the effect of O⁶BG/BCNU in in vivo selection, transgene integration in bone marrow Lin− cells was analyzed at the end of the study, i.e., at week 20 in secondary recipients. Linear amplification-mediated PCR (LAM-PCR) followed by deep sequencing showed a random distribution pattern of integration sites in the mouse genome (FIG. 5A). Data pooled from 5 mice demonstrated 2.23% integration into exons, 31.58% into introns, 65.17% into intergenic regions, and 1.04% into untranslated regions (FIG. 5B). The level of randomness of integration was 99% without preferential integration in any given window of the whole mouse genome (FIG. 5C). This indicates that in vivo selection and further expansion of cells in secondary recipients did not result in the emergence of dominant integration sites (FIG. 5D). qPCR was used to measure, on average, two γ-globin cDNA copies per bone marrow cell in a population containing both transduced and non-transduced cells. The integrated transgene copy number was then quantified on a single-cell level. To do this, bone marrow Lin− cells from week 18 mice were plated in methylcellulose, isolated individual progenitor colonies, and performed qPCR on genomic DNA. In transgene-positive colonies (n=113), 86.7% of colonies had 2 or 3 integrated copies (FIG. 5E and FIG. 6). Four copies were found in 6.2% of colonies, 8 copies in 1.78%. 0.88% of colonies had either 13, 10, 7, 6, or 5 integrated vector copies.

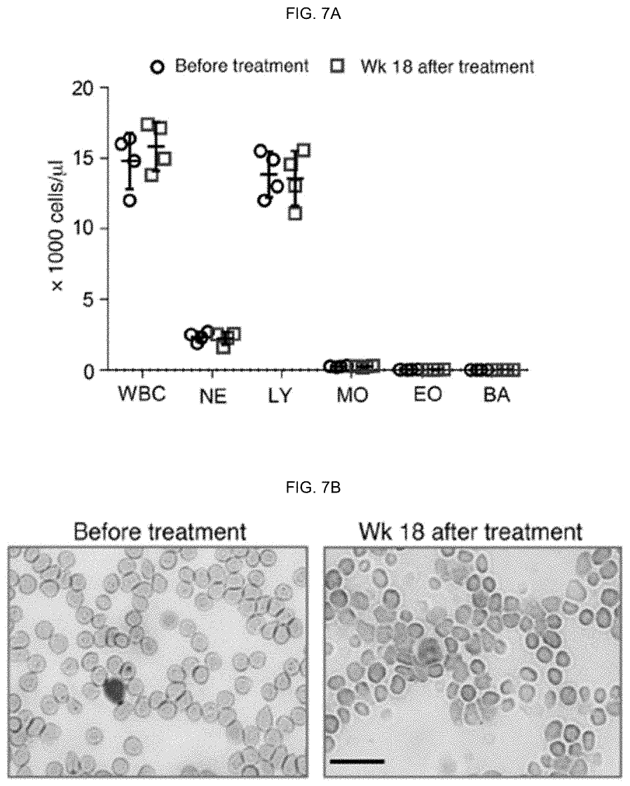

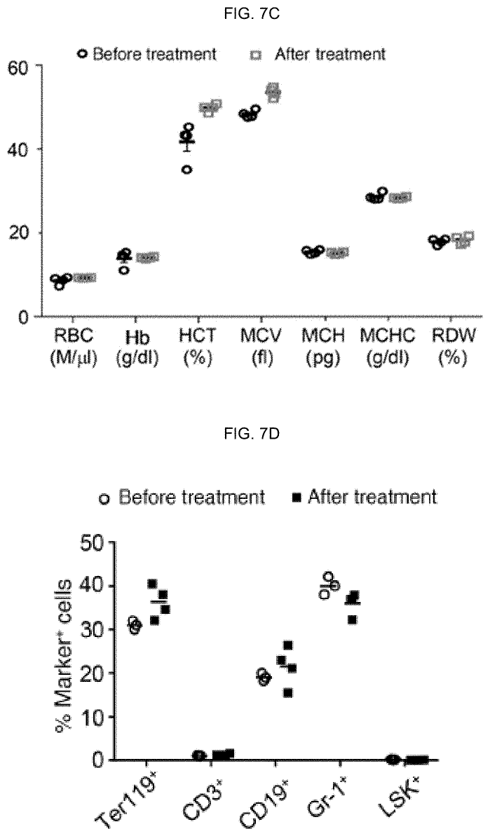

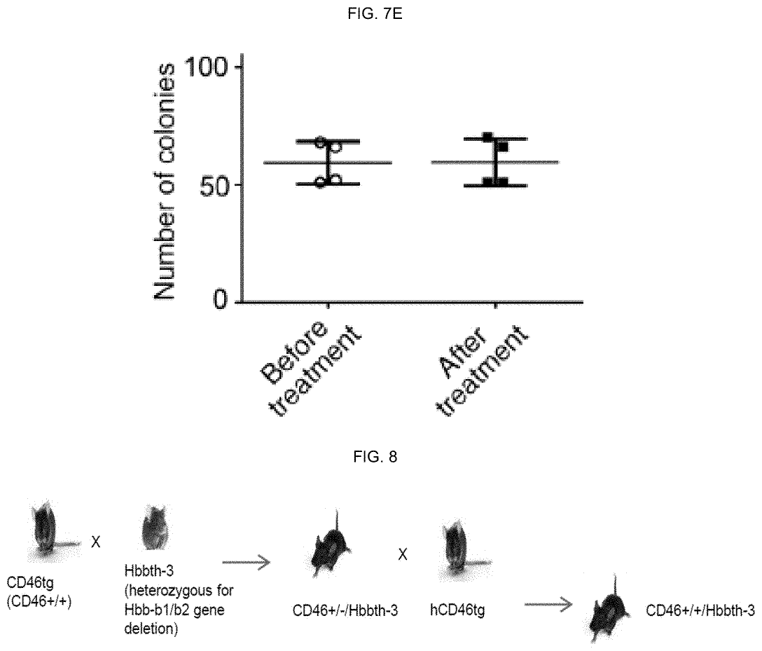

No alterations in blood cell counts were found at the end the study (week 18) (FIG. 7A). Analysis of RBC parameters did not show abnormalities (FIGS. 7A-7C). Composition of Lin+ fractions in the bone marrow was similar in mice before and after treatment (week 18) mice (FIG. 7D). The levels of Lin-Sca1+cKit+ (LSK) HSPCs (FIG. 7D, last lane) and progenitor colony-forming cells (FIG. 7E) were also comparable in both groups.

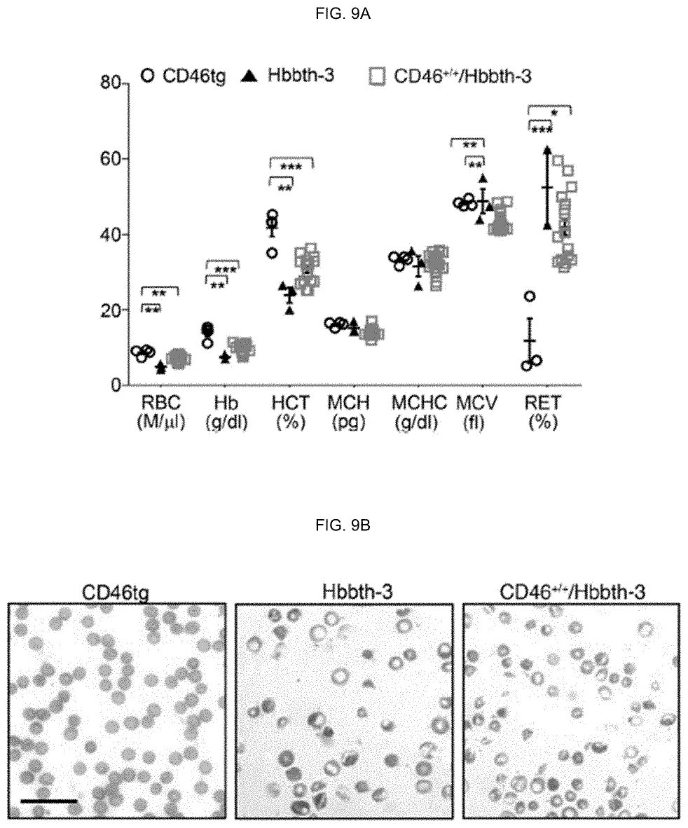

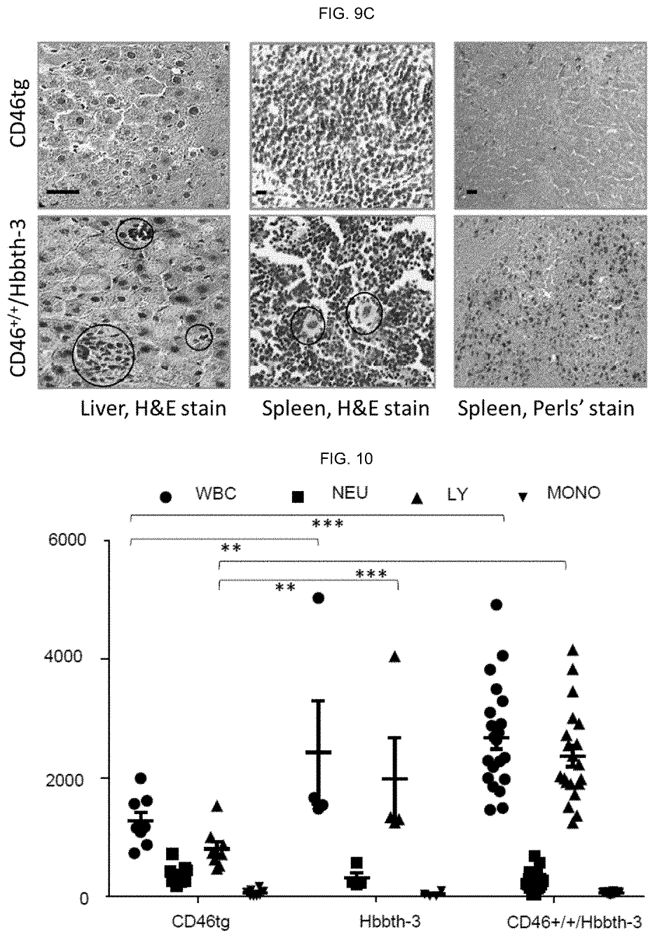

Generation of the CD46+/+/Hbbth-3 mouse model expressing human CD46 and resembling human thalassemia intermedia. HDAd5/35++ vectors require human CD46 for infection. In order to develop a thalassemic mouse model for in vivo HSPC transduction studies, CD46tg (CD46+/+) mice were bred with Hbbth-3 mice heterozygous for the mouse Hbb-β1 and -β2 gene deletion (Yang et al., Proc Natl Acad Sci USA. 92(25):11608-11612, 1995). (The homozygous state is lethal in utero or early postnatally.) Hbbth-3 mice represent a viable form of thalassemia, resembling human thalassemia intermedia. F1 hybrid mice were backcrossed with CD46+/+ mice to generate CD46+/+/Hbbth-3 mice (FIG. 8). These mice displayed a thalassemic phenotype. Compared with parental CD46tg mice, CD46+/+/Hbbth-3 mice had significantly decreased RBC numbers (7.1±0.1 vs. 8.63±0.29 M/μl); lower hemoglobin (9.7±0.18 vs. 13.9±0.63 g/dl), hematocrit (30.7%±0.46% vs. 41.7%±1.48%), mean corpuscular hemoglobin (13.9±0.14 vs. 16.1±0.23 g/dl), and mean corpuscular volume (43.03±0.22 vs. 48.35±0.9 fl); and increased RBC distribution width (42.9%±0.29% vs. 25.3%±0.79%); and showed overt reticulocytosis (42.4%±1.43% vs. 11.8%±3.7%) (FIG. 9A). The red cell morphology in blood smears was characterized by hypochromia, widely varying sizes and shapes (anisopoikilocytosis), and cell fragmentation, similarly to the morphology of the Hbbth-3 mouse blood smears and in sharp contrast to the normocytic red cell appearance of CD46tg mice (FIG. 9B). Likewise, histological analysis of liver and spleen from CD46^{+/+}/Hbbth-3 mice revealed foci of extramedullary hemopoiesis containing clusters of erythroid precursors or megakaryocytes (FIG. 9C, bottom left and bottom middle panels), while Perls' staining demonstrated marked parenchymal iron deposition (FIG. 9C, bottom right panel) as opposed to absent or limited extramedullary hemopoiesis and iron accumulation in tissue sections from parental CD46tg mice (FIG. 9C, top panels). These characteristics of CD46^{+/+}/Hbbth-3 mice recapitulate the human disease and support the validity of such a model for subsequent experiments. Notably, the thalassemic phenotype in the CD46^{+/+}/Hbbth-3 model was also characterized by quantitative differences in lineages other than the erythroid lineage, as indicated by the elevated numbers of total WBCs (FIG. 10).

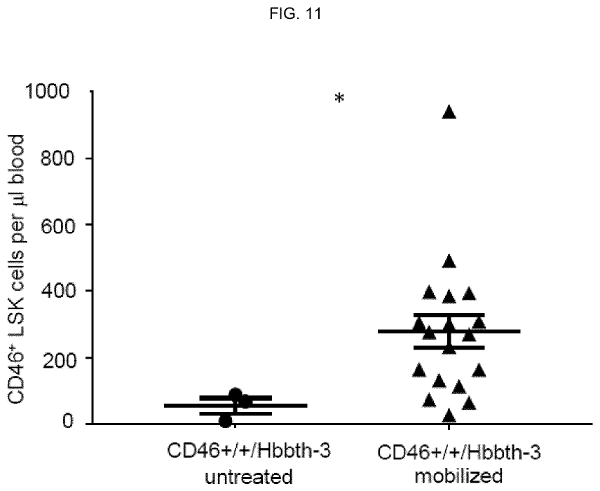

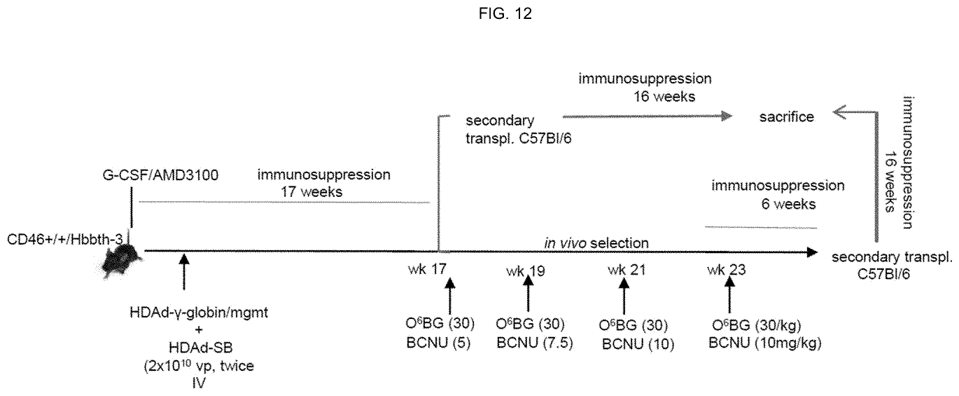

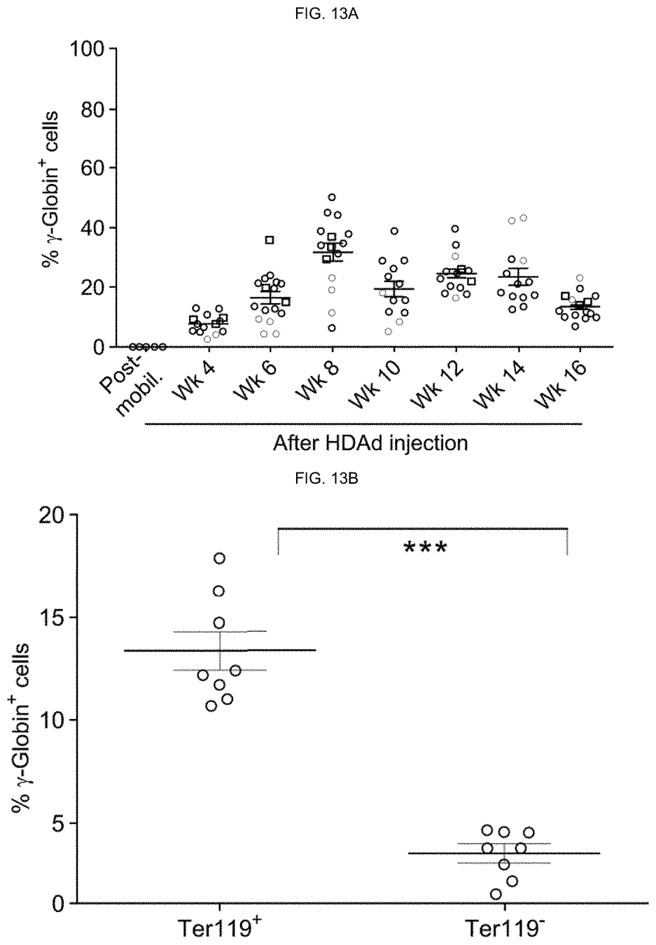

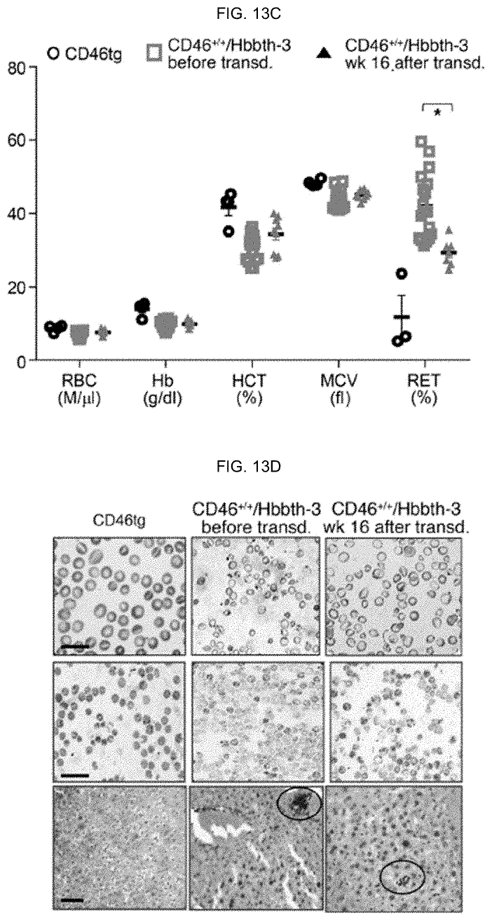

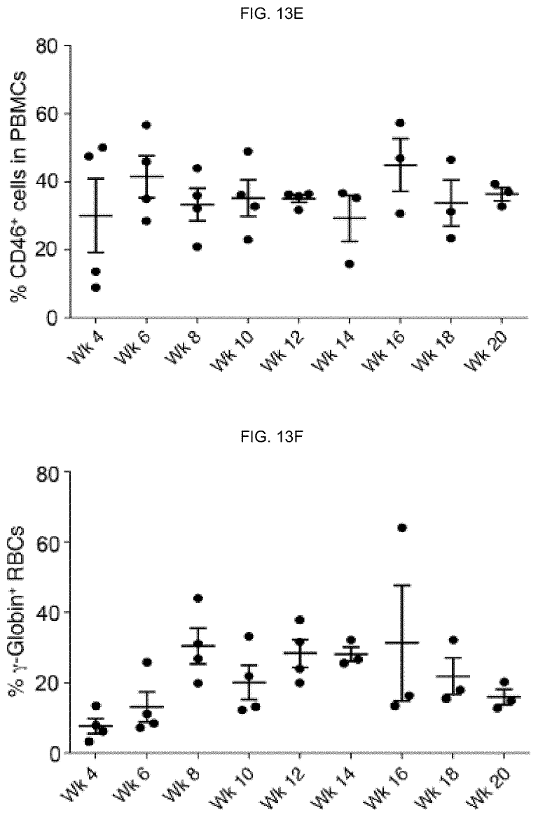

HSPC in vivo transduction with HDAd-γ-globin/mgmt plus HDAd-SB followed by in vivo selection in CD46^{+/+}/Hbbth-3 mice results in high, stable, and long-term expression of γ-globin. It was determined whether the in vivo transduction approach could ameliorate the characteristic disease parameters of the CD46^{+/+}/Hbbth-3 thalassemia mouse model. A modified G-CSF/AMD3100 mobilization scheme that was previously validated in Hbbth-3 mice (Psatha et al., Hum Gene Ther Methods. 2014; 25(6):317-327) yielded high numbers of LSK cells in the peripheral blood 1 hour after the last plerixafor (AMD3100) injection (FIG. 11), i.e., at the time point when HDAd-γ-globin/mgmt and HDAd-SB were injected intravenously. Mice received immunosuppression to avoid responses against the human γ-globin and MGMT proteins (FIG. 12). Considering a report that after ex vivo lentivirus vector gene therapy, genetically corrected erythroblasts have a survival advantage and undergo in vivo selection in Hbbth-3 mice (Miccio et al., Proc Natl Acad Sci USA 105(30):10547-10552, 2008), it was initially planned to conduct the study without O⁶BG/BCNU treatment. Average γ-globin⁺ RBC percentages reached 31.19%±2.7% at week 8 after in vivo transduction of CD46^{+/+}/Hbbth-3 mice but declined to 13.14%±0.4% by week 16. At this time, mice were split into 2 groups. Half of the mice were used for blood and bone marrow analysis (group 1: without in vivo selection) and as donors for secondary recipients, while the study was continued with the other group involving O⁶BG/BCNU in vivo selection (group 2: with in vivo selection) (see FIG. 12). At week 16, group 1 showed γ-globin expression in 13% of peripheral RBCs (FIGS. 13A, 13B). This level of γ-globin marking resulted in a significant reduction in the percentage of peripheral blood reticulocytes (FIG. 13C, last lane). However, it did not suffice to improve other RBC parameters, including RBC morphology and extramedullary hemopoiesis (FIGS. 13C, 13D). The level of primary γ-globin marking was maintained over 20 weeks in secondary C57BL/6 recipients that were myelo-conditioned with busulfan before transplantation (FIGS. 13E, 13F). This indicates that long-term-repopulating HSPCs were transduced.

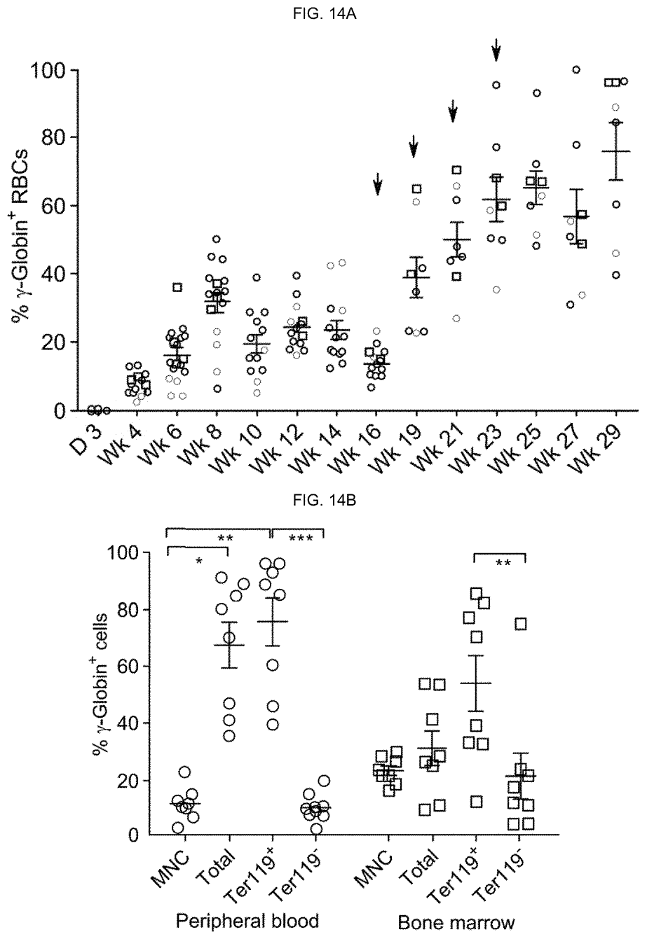

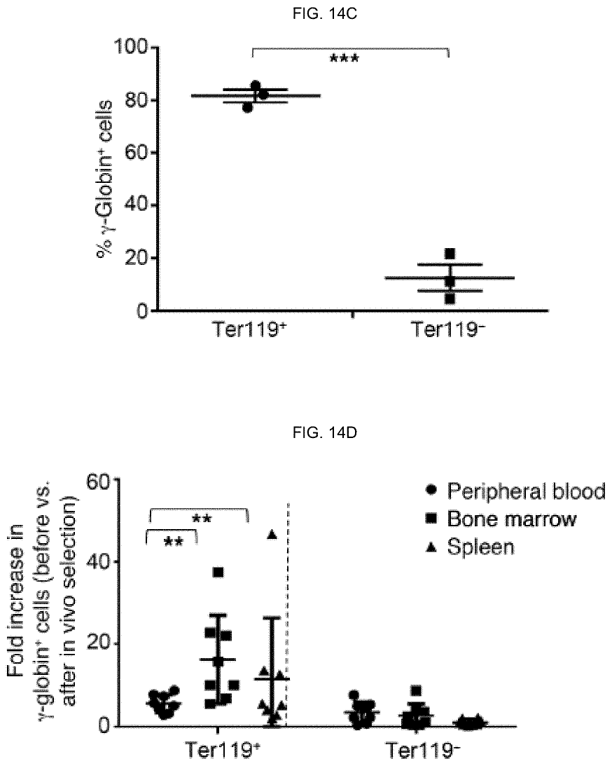

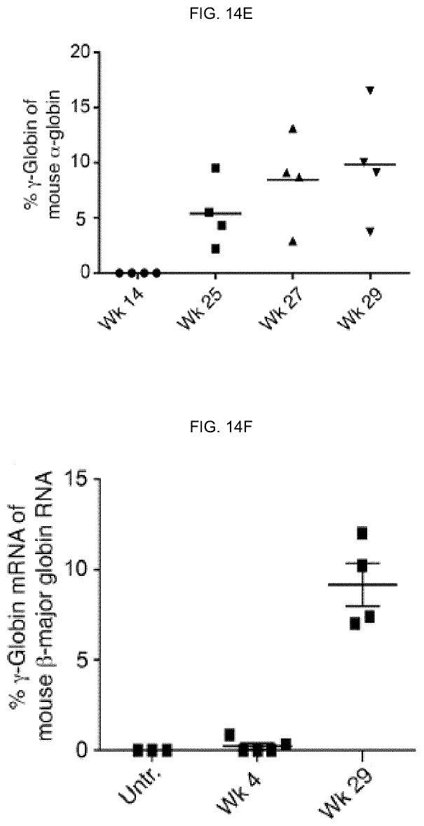

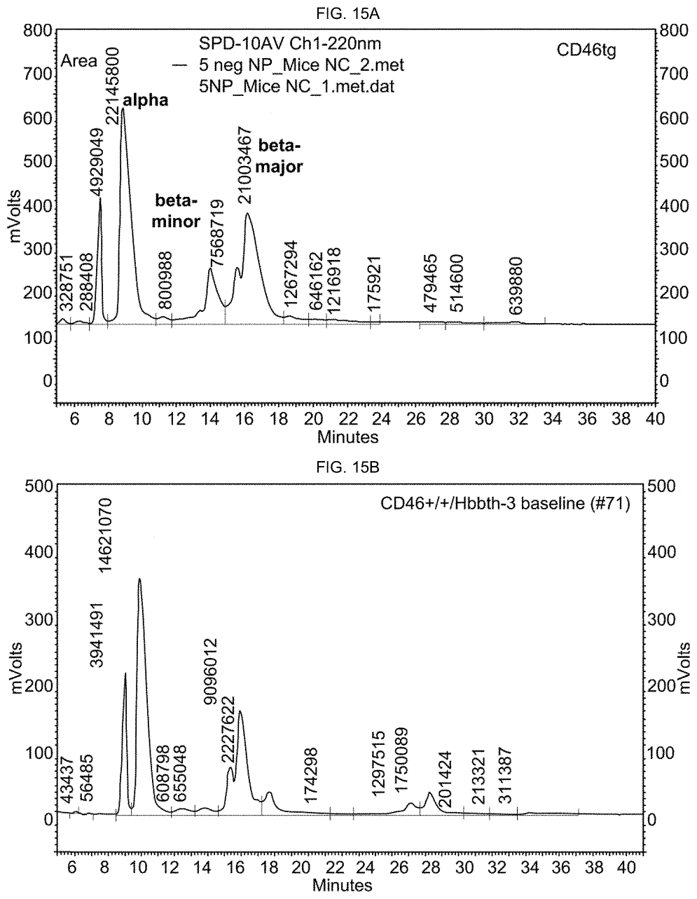

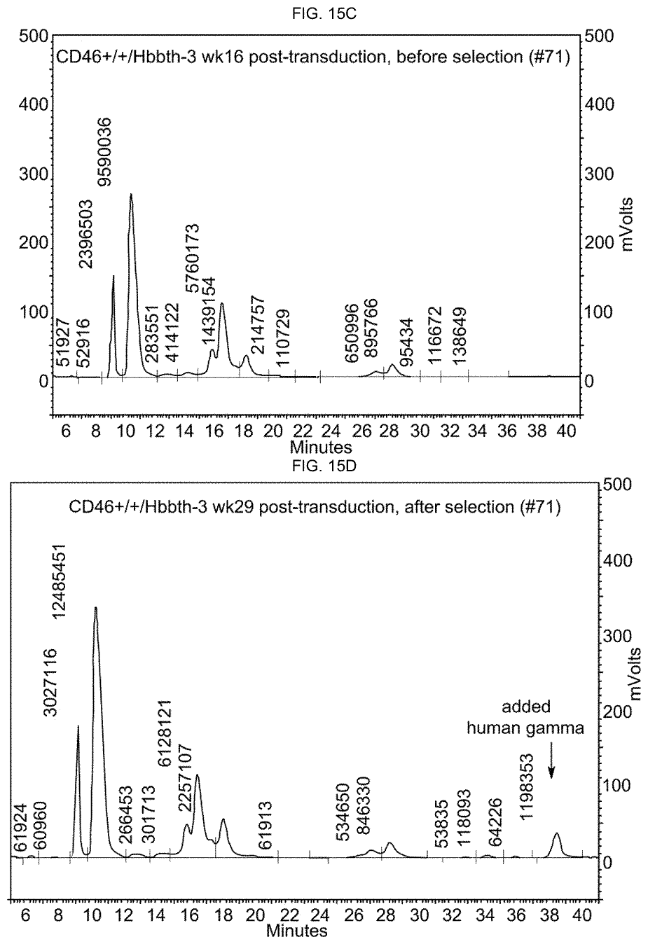

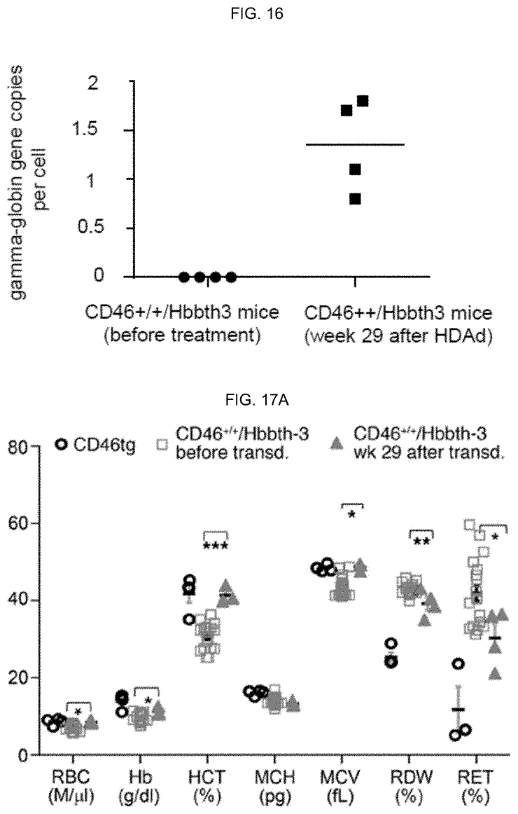

In group 2, 4 cycles of in vivo selection resulted in a 6-fold increase in the percentage γ-globin⁺ RBCs reaching an average of 76% at week 29 (FIG. 14A). γ-Globin expression was erythroid-specific as indicated by analysis of γ-globin expression in gated or immunomagnetically isolated Ter119+ erythroid cells by flow cytometry as compared with Ter119− cells (FIG. 14B, FIG. 14C). In agreement with other studies (Miccio et al., Proc Natl Acad Sci USA. 105(30):10547-10552, 2008, Zhao et al., Blood. 113(23):5747-5756, 2009), selection occurred at the level of (nucleated and proliferating erythroid) progenitors before they exit the bone marrow (or spleen) and lose their nucleus. This is reflected in an increase of γ-globin⁺ Ter119⁺ cells in the bone marrow and spleen that occurred predominantly after versus before in vivo selection (FIG. 14D). However, the overall increase of γ-globin⁺ marking in Ter119⁺ cells in peripheral blood (where enucleated RBCs predominate) (FIG. 14B) is probably due to the additive effect of the “natural” in vivo selection provided by the thalassemic background. The human γ-globin over mouse α-globin ratio in RBCs measured by HPLC increased from almost undetectable levels at week 14 to 10% at week 29 (FIGS. 14E and 15; see CD46^{+/+}/Hbbth-3 mouse at baseline (FIG. 15B), week 16 (FIG. 15C) and week 29 (FIG. 15D, and CD46tg control (FIG. 15A)). Similarly, the level of γ-globin mRNA in blood cells of treated mice increased, translating into 10% human γ-globin mRNA of mouse β-globin mRNA at week 29 (FIG. 14F). 1.5 γ-globin gene copies per cell were measured in treated CD46^{+/+}/Hbbth-3 mice at week 29 after in vivo transduction (FIG. 16).

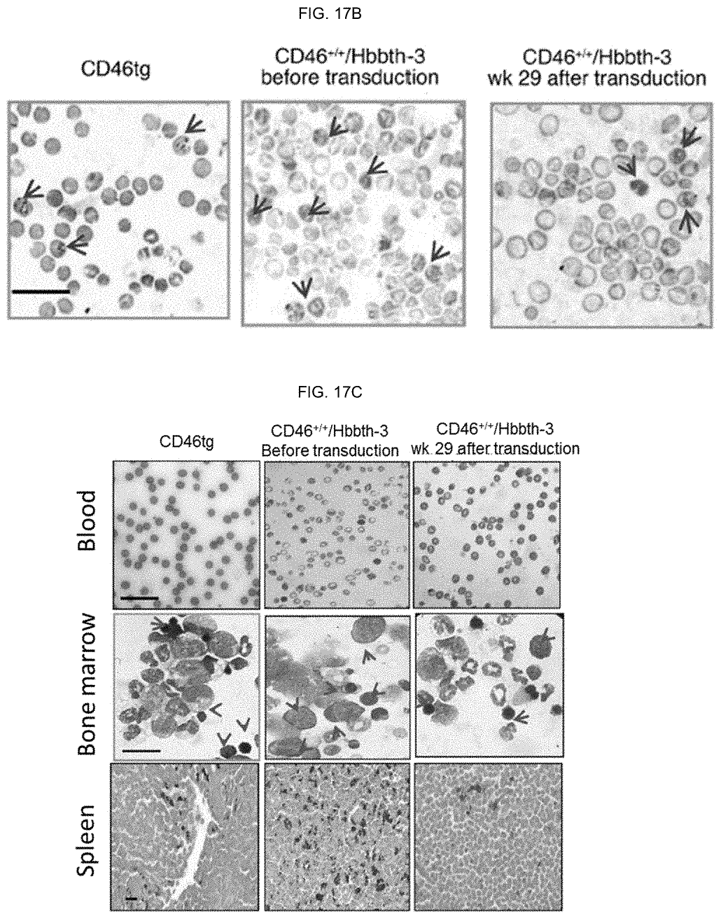

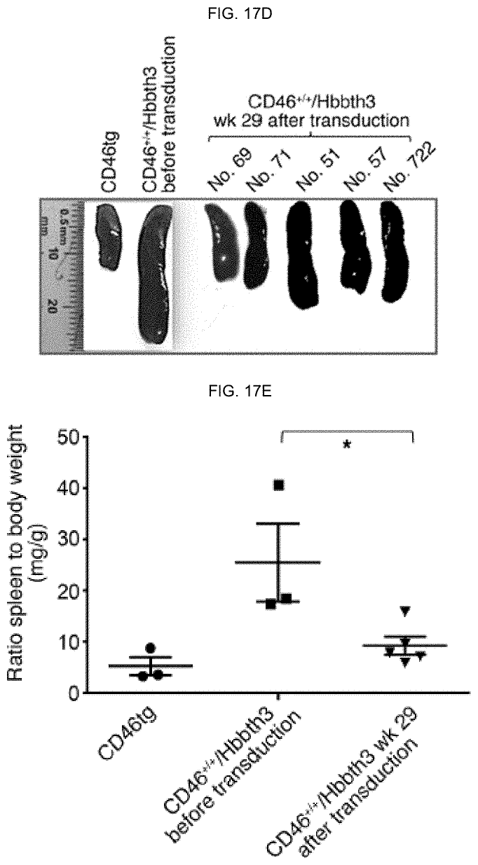

Reversal of the thalassemic phenotype of CD46^{+/+}/Hbbth-3 mice after in vivo transduction/selection. Six weeks after the last dose of O⁶BG/BCNU treatment, CD46^{+/+}/Hbbth-3 mice were sacrificed, and hematopoietic tissues were harvested for analysis. Hematological parameters at week 29 after in vivo transduction were significantly improved over baseline (FIG. 17A) (RBCs: 8.53±0.16 vs. 7.1±0.13 M/μl, P=0.01; hemoglobin: 11.27±0.39 vs. 9.7±0.18 g/dl, P=0.05; hematocrit: 41.37%±0.81% vs. 30.7%±0.46%, P=0.00001; mean corpuscular volume: 48.63±0.36 vs. 43.5±0.38 fl, P=0.003; RBC distribution width: 39.5%±0.8% vs. 43%±0.3%, P=0.006; reticulocytes: 31.13%±3.17% vs. 42.4%±1.43%, P=0.05), and for specific red cell indices (hematocrit [HCT], RBCs, mean corpuscular volume), levels were indistinguishable from their control CD46tg counterparts, suggesting near to complete phenotypic correction. Reticulocyte staining of blood smears demonstrated an impressive 3-fold reduction of reticulocyte numbers in treated CD46^{+/+}/Hbbth-3 mice with the highest percentage of γ-globin⁺ RBCs (FIG. 17B). Indicative of the reversal of the thalassemic phenotype in peripheral blood smears of the treated CD46^{+/+}/Hbbth-3 mice, the hypochromic, highly fragmented and anisopoikilocytic baseline RBCs were replaced by near-normochromic, well-shaped RBCs less variant in size (FIG. 17C, top panels). In contrast to the blockade of erythroid lineage maturation in bone marrow of CD46^{+/+}/Hbbth-3 mice, represented by the prevalence of pro-erythroblasts and basophilic erythroblasts, in cytospins from control and treated CD46^{+/+}/Hbbth-3 mice, maturing erythroblasts predominated and were represented by polychromatic and orthochromatic erythroblasts (FIG. 17C, middle panels). Intense parenchymal hemosiderosis was observed in the untreated CD46^{+/+}/Hbbth-3 mice, whereas only limited iron accumulation in the CD46tg and the treated CD46^{+/+}/Hbbth-3 mice could be detected (FIG. 17C, bottom panels). Accordingly, spleen size, a measurable characteristic of compensatory hemopoiesis, was markedly reduced in treated animals (FIGS. 17D, 17E).

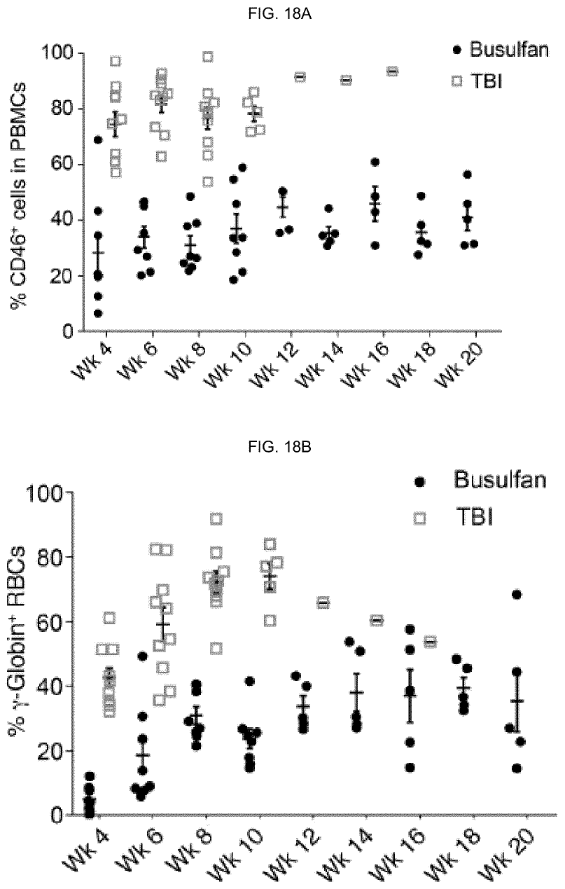

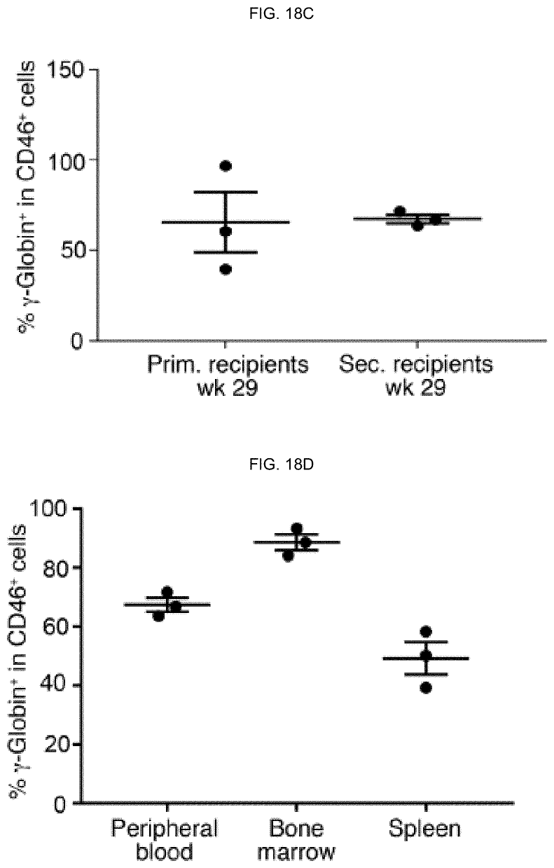

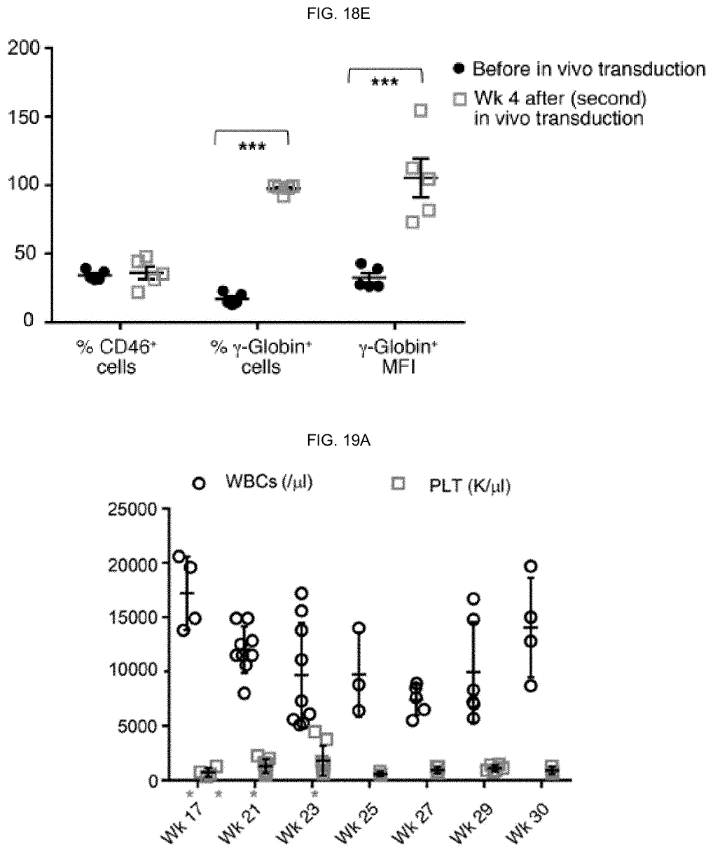

In order to determine whether the combined in vivo transduction/selection approach resulted in genetic modification of primitive HSCs, bone marrow cells from treated CD46^{+/+}/Hbbth-3 mice harvested at week 29 (after transduction) were transplanted into C57BL/6 secondary recipients after either sublethal busulfan treatment or lethal total-body irradiation (TBI) (FIGS. 18A, 18B). Although, as expected, the engraftment rates in mice that received TBI were higher than those in busulfan-treated animals, the levels of expression adjusted to the engraftment levels did not show significantly different frequencies of γ-globin⁺ RBCs. The fact that more than 75% of transplant-derived (CD46+) erythrocytes were γ-globin⁺ at week 20 after secondary transplantation and with a marking rate similar to that found in primary treated mice at week 29 (FIGS. 18C, 18D) under the competitive conditions generated by the submyeloablative busulfan conditioning in a normal recipient background (in which the HDAd-γ-globin/HDAd-SB-transduced CD46^{+/+}/Hbbth-3 HSPCs had no selective advantage) further supports the conclusion that the approach results in the genetic correction of long-term repopulating HSCs. Moreover, secondary, busulfan-conditioned C57BL/6 recipients at week 20 after transplant that were submitted to mobilization and in vivo transduction demonstrated a remarkable enrichment in γ-globin-expressing cells and a significant increase in expression/MFI (FIG. 18E).

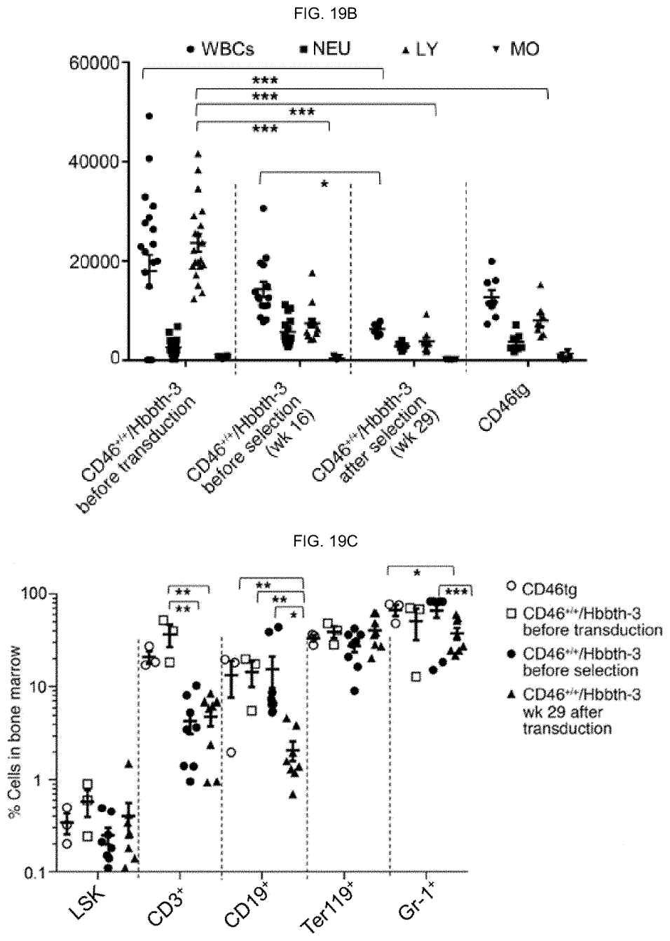

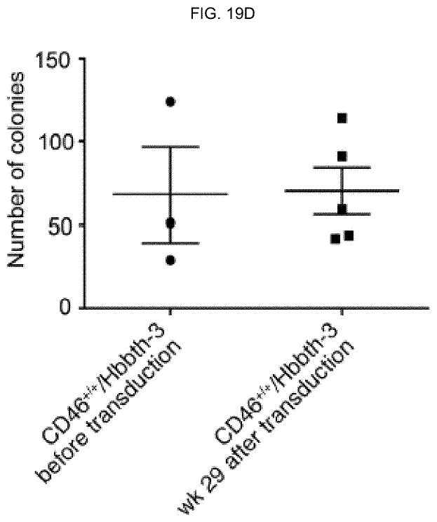

Safety of in vivo HSPC transduction with HDAd-γ-globin/mgmt plus HDAd-SB followed by O⁶-BG/BCNU in vivo selection. In the mouse studies, the procedure was well tolerated. No overt hematological abnormalities were observed. At time of sacrifice, 6 weeks after the last O⁶-GB/BCNU dose, all hematological values were within normal ranges, although the total WBC counts were lower compared with levels before in vivo selection, suggesting a cytoreductive effect of drug treatment on WBCs—in particular, lymphocytes (FIGS. 19A, 19B). This effect was also reflected in the reduced frequency of CD3+, CD19+, and Gr-1+ cells in bone marrow as compared with their untreated or preselection counterparts (FIG. 19C). Notably, even at their nadir (week 25-27; 2-4 weeks after the last O⁶BG/BCNU injection), the WBCs and platelets never reached aplasia levels (i.e., neutrophils <1,000/μl, platelets <20,000/p1), and the WBCs started to recover by week 30 (7 weeks after the last O⁶BG/BCNU injection). This together with the observation that in the CD46tg model, WBCs and lymphocyte counts returned to pretreatment levels 10 weeks after the last O⁶BG/BCNU injection (FIG. 7A), suggests that the cytoreductive effect of the in vivo selection drugs is mild and transient. Importantly, the bone marrow cell composition in the percentage of LSK and Ter119⁺ cells, as well as the colony-forming potential of bone marrow cells, was not affected by the in vivo transduction/selection of HSPCs (FIGS. 19C, 19D).

Discussion Despite the unequivocal clinical progress in ex vivo HSPC gene therapy of hemoglobinopathies, the need for myeloablative conditioning in order to reach clinically relevant HSPC engraftment rates is a major limitation. Furthermore, the technical complexity allows the implementation of such treatment in only a small number of specialized and/or accredited centers. The in vivo HSPC gene therapy approach that has been developed does not require myeloablation and HSPC cell transplantation and therefore makes HSPC gene therapy for thalassemia safer and more accessible. The central idea of the approach is to mobilize HSPCs from the bone marrow and, while they circulate at high numbers in the periphery, transduce them with an intravenously injected HSPC-tropic HDAd5/35++gene transfer vector system. The novel features of the HDAd5/35++ vector system include (a) CD46-affinity-enhanced fibers that allow for efficient transduction of primitive HSCs while avoiding infection of nonhematopoietic tissues after intravenous injection, (b) an SB100X transposase-based integration system that functions independently of cellular factors and mediates random transgene integration without a preference for genes, and (c) an MGMT^{P140K} expression cassette mediating selective survival and expansion of progeny cells without affecting the pool of transduced primitive HSCs by short-term treatment with low-dose O⁶BG/BCNU (Wang et al., Mol Ther Methods. Clin Dev. 8:52-64, 2018). Additional features that distinguish HDAd5/35++ vectors from currently used SIN-lentiviral (SIN-LV) vectors include their large (30 kb) insert capacity, which, in this study, was used to incorporate a micro-LCR/β-promoter-driven γ-globin gene and an EF1A promoter-driven MGMT^{P140K} gene with a size of 11.8 kb. Furthermore, the production of HDAd5/35++ vectors does not require large-scale plasmid transfections and yields more than 3×10¹² infectious particles per liter spinner culture. Notably, the yields of SIN-LV vectors used in clinical trials for hemoglobinopathies are at least 2 orders of magnitude lower.

Efficacy of the in vivo approach. In contrast to HSPC gene therapy of other genetic diseases (i.e., X-linked SCID, Cavazzana-Calvo et al., Science. 288(5466):669-672, 2000; ADA-SCID, Gaspar et al., Sci Transl Med. 3(97):97ra80. 2011; or Wiskott-Aldrich syndrome, Aiuti et al., Science. 341(6148):1233151, 2013) where stable transduction of less than 1% of HSPCs provides a significant clinical benefit, phenotypic correction of hemoglobinopathies in patients requires at least 20% corrected erythroid precursors (Persons et al., Blood. 97(10):3275-3282, 2001, Andreani et al., Blood.; 87(8):3494-3499, 1996, Negre et al., Blood. 117(20):5321-5331, 2011). In murine models for hemoglobinopathies, γ-globin expression at 15% of the total α-globin mRNA was sufficient for therapy (Persons et al., Blood. 2001; 97(10):3275-3282, McColl et al., Blood Med. 7:263-274, 2016, Pestina et al., Mol Ther. 17(2):245-252, 2009). In this study, after in vivo transduction/selection, more than 60% of bone marrow erythroblasts expressed γ-globin in the in vivo transduced CD46tg and CD46^{+/+}/Hbbth-3 models (FIGS. 2C and 14A). This translated into 40%-97% circulating γ-globin-expressing RBCs (FIGS. 2D and 14B). Importantly also, in both animal models, sustained γ-globin marking in RBCs was demonstrated in secondary recipients, suggesting that primitive, long-term repopulating HSCs were initially transduced by the vector system.

The qPCR studies detected 2 to 3 integrated transgene copies per cell in the overwhelming majority of bone marrow cells. In agreement with earlier studies (Zhao et al., Blood. 113(23):5747-5756, 2009, Zielske et al., Mol Ther. 9(6):923-931, 2004), it was not found that in vivo selection selected for high-copy-number clones. Taking into account the genome-wide integration site analysis, 1,000 originally transduced HSCs were projected. Considering that mice have 10,000 to 20,000 HSCs (Abkowitz et al., Blood. 100(7):2665-2667, 2002; Chen et al., Blood. 107(9):3764-3771, 2006), this would mean that the vector system targeted 5%-10% of HSCs, which would be a solid basis for a polyclonal reconstitution of hematopoiesis after in vivo selection and for a long-term therapeutic effect.

In the thalassemia intermedia model, a near-complete phenotypic correction was achieved. Key hematological parameters (HCTs, RBCs, mean corpuscular volume) were indistinguishable from their counterparts in “healthy” (parental CD46tg) mice. The degree of correction of RBC indices and morphology correlated with the level of γ-globin-expressing cells in individual mice. Peripheral RBCs and erythroid bone marrow precursor cells resembled those of healthy mice in both morphology and the maturation process. Extramedullary hematopoiesis and parenchymal iron deposition regressed, and spleen size was significantly reduced. The thalassemic phenotype in the CD46+/+/Hbbth-3 model was also characterized by leukocytosis/lymphocytosis (FIG. 10). (Leukocytosis/lymphocytosis is also often present in splenectomized thalassemia/sickle cell disease patients or patients with functional, disease-associated asplenia; Brousse et al, Br J Haematol. 166(2):165-176, 2014). Interestingly, WBC counts in CD46++/Hbbth-3 mice returned to levels of “healthy” CD46tg mice at week 29 after in vivo transduction (FIG. 19A). This effect suggests that the reversal of the thalassemic phenotype by the approach extends beyond the erythroid compartment, resulting in normalization of WBCs, and most likely overall spleen function.

Notably, in contrast to the study in CD46tg mice, in the context of a thalassemic background and in the absence of O⁶BG/BCNU treatment, 13% of γ-globin⁺ RBCs were circulating in peripheral blood of CD46+/+/Hbbth-3 mice, and this level was maintained long-term in secondary recipients. This indicates that γ-globin gene expression conferred a survival advantage to thalassemic genetically modified erythroid precursors similar to what was reported with ex vivo lentivector HSPC gene therapy in a mouse model of thalassemia major (Micco et al, Proc Natl Acad Sci USA. 105(30):10547-10552, 2008). However, the phenotypic correction in the thalassemia mouse model required O⁶BG/BCNU treatment. This suggests that, if required because of low globin marking, the inducible in vivo selection system allows for salvaging of the therapeutic efficacy by an easy pharmacological intervention.

To increase the level of γ-globin in murine thalassemia models further, the following possibilities may be considered: (a) The ratio of HDAd-SB to HDAd-γ-globin/mgmt vectors could be changed from 1:1 to 1:3 to increase the number of integrated transgene copies per cell (Zhang et al, PLoS One. 8(10):e75344, 2013). (b) It is also planned to use a 26.1-kb version of β-globin LCR to drive γ-globin expression to minimize transgene integration position effects (Wang et al, J Virol. 79(17):10999-11013, 2005). (c) In addition to the SB100X-based γ-globin gene addition system, the HDAd5/35++ vector could accommodate a CRISPR/Cas9 to disrupt γ-globin suppressor regions and reactivate the endogenous γ-globin genes (Li et al., Blood. 131(26):2915-2928, 2018).

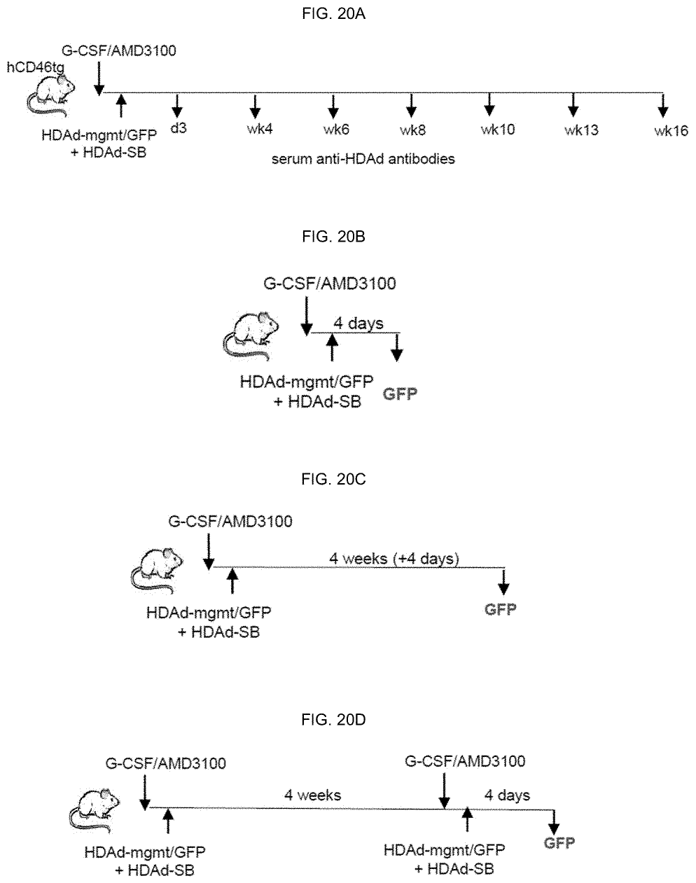

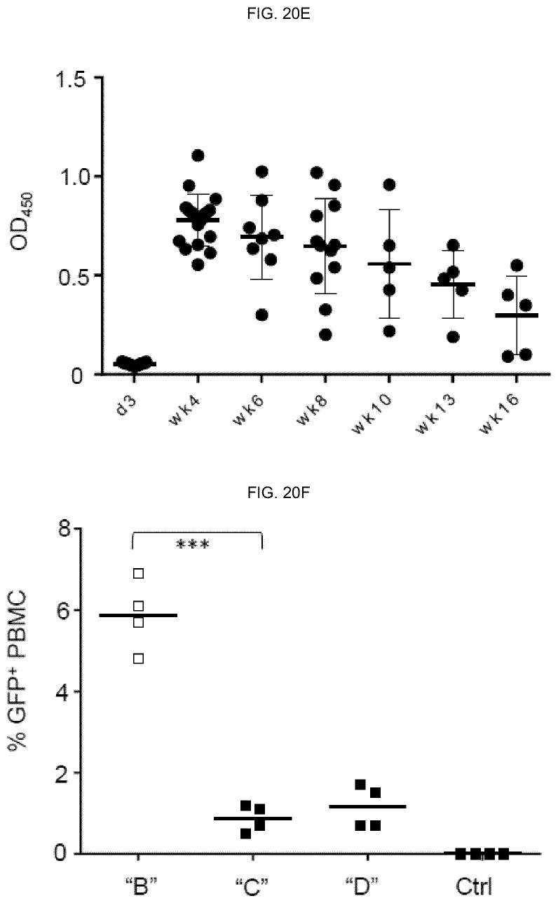

To evaluate the relationship of time from mobilization and expression, an HDAd-mgmt/GFP vector+an HDAd-SB vector were administered to hCD46tg mice after mobilization with G-CSF and AMD3100. Serum anti-HDAd antibodies were measured as shown in FIGS. 20A and 20E. GFP was measured 4 days or 4 weeks and 4 days after mobilization (FIGS. 20B (“B”) and 20C (“C”)). A second round of mobilization and HDAd injection (4 weeks after the first round; FIG. 20D). Results are shown in FIG. 20F. The second round of mobilization (FIG. 20D; “D”) did not result in transduction of peripheral blood cells because of the development of neutralizing serum antibodies against the virus. However, as the in vivo transduction studies in secondary transplant recipients indicate (FIG. 18E), if the development of anti-HDAd antibodies could be pharmaceutically blocked, a second treatment could increase both the percentage of γ-globin⁺ RBCs and the γ-globin expression level/MFI.

Safety of the in vivo HSPC transduction/selection approach. This approach abrogates the need for myeloablation/conditioning and its associated toxicity, while it effectively targets HSPCs in the unconditioned host by simple intravenous and subcutaneous substance/vector injections. Importantly, the procedure has been well tolerated in all animals involved in this study.

Concerning the HSPC mobilization based on G-CSF/AMD3100 (plerixafor), the approach has been clinically proven safe and efficacious and is routinely used for HSPC mobilization and collection by leukapheresis in all running trials for thalassemia major (Psatha et al., Curr Gene Ther. 17(5):364-378, 2017, Karponi et al., Blood. 126(5):616-619, 2015). As an alternative to the mobilization regimen used in this study, other approaches may involve the continuous blockade of CXCR4 by small synthetic molecules to achieve a more efficient mobilization of HSPCs (Karpova et al., Blood. 129(21):2939-2949, 2017).

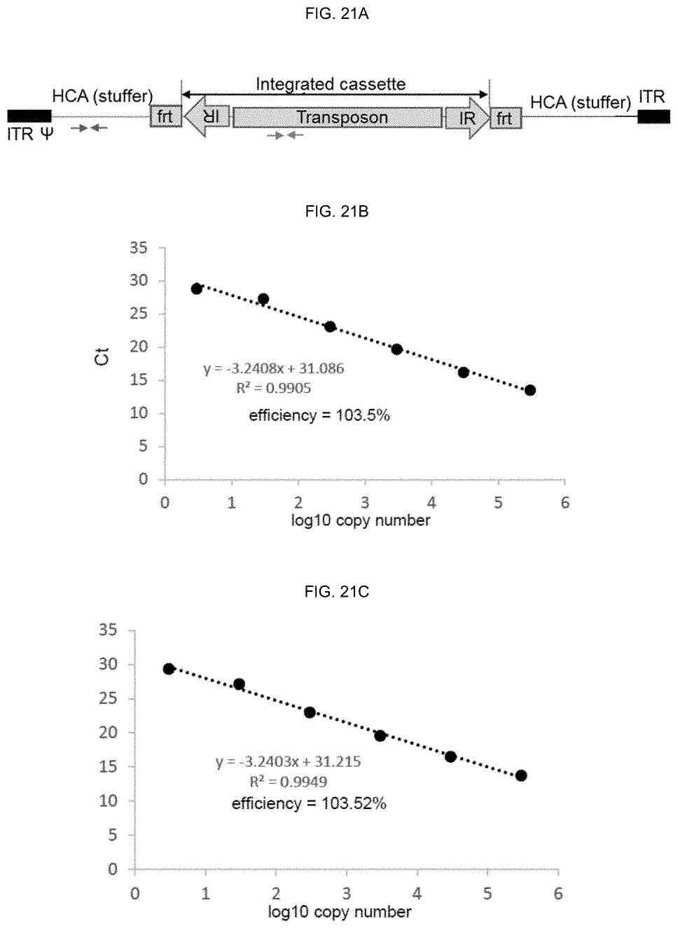

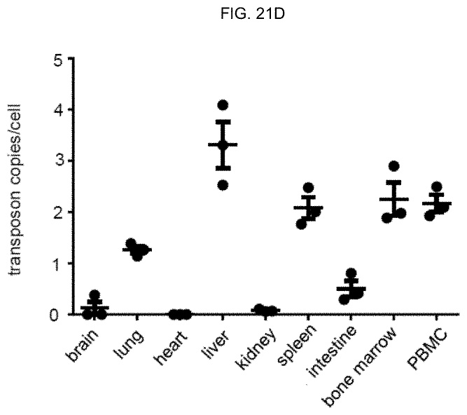

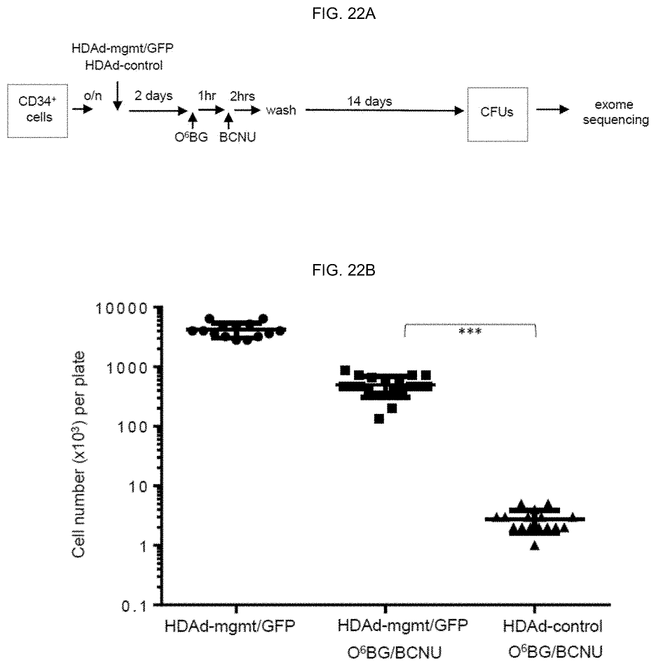

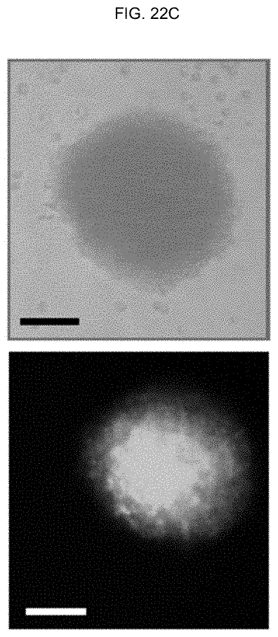

The intravenous injection of HDAd5/35++ vectors does not result in transgene expression in tissues other than the mobilized HSPCs and PBMCs in CD46tg mice at day 3 after injection (Richter et al., Blood. 128(18):2206-2217, 2016). This was in agreement with early studies in baboons with intravenously injected first-generation CD46-targeting Ad5/35 and Ad5/11 vectors (Ni et al., Blood. 128(18):2206-2217, 2016). A potential explanation for this tropism is that CD46 receptor density and accessibility are not sufficiently high in nonhematopoietic tissues to allow for efficient viral transduction (Richter et al., Blood. 128(18):2206-2217, 2016; Ong et al., Exp Hematol. 34(6):713-720, 2006). Here, the number of integrated transgene copies per cell were measured in different tissues at week 18 after in vivo transduction/selection using a transposon vector (FIG. 21A). Efficiency relative to copy number is presented in FIGS. 21B and 21C. Transposon copies integrated per cell in various tissues are shown (FIG. 21D). The copy number in bone marrow, PBMCs, and spleen was 2.5. Integrated transgenes were also detected in the genomic DNA from liver, lung, and intestine. Previous studies with a GFP vector system have shown that the signals in these organs originate from infiltrating blood cells and/or residential macrophages (Richter et al., Blood. 2016; 128(18):2206-2217).

Intravenous injection of HDAd vectors (but also other viral vectors) is associated with the release of proinflammatory cytokines (Atasheva et al., Curr Opin Virol. 21:109-113, 2016, Grieg et al., Mol Ther Methods Clin Dev. 3:16079, 2016), which can, however, efficiently be blocked by pretreatment with glucocorticoids the day before virus injection (Seregin et al., Mol Ther. 17(4):685-696, 2009) or vector dose fractionation (Illingworth et al., Mol Ther Oncolytics. 5:62-74, 2017). Good safety profiles of intravenously injected oncolytic adenoviruses have been documented in dozens of clinical trials, including a trial with a CD46-targeting oncolytic adenovirus (Garcia-Carbonero et al., J Immunother Cancer. 5(1):71, 2017).

Regarding the safety of in vivo selection and the concern that O⁶BG/BCNU-stimulated proliferation may deplete the reservoir of long-term quiescent HSPCs, studies with large-animal models have provided evidence for long-term multilineage selection without HSPC exhaustion or emergence of dominant clones (Beard et al., J Clin Invest. 120(7):2345-2354, 2010, Neff et al., J Clin Invest. 112(10):1581-1588, 2003). In these models, the hematopoietic and the extramedullary toxicity profile was acceptable. In the present study and the previous mouse studies (Wang et al., Mol Ther Methods Clin Dev. 8:52-64, 2018, Li et al., Blood. 131(26):2915-2928, 2018), in vivo selection was well tolerated without myelosuppression. No changes in the frequency of bone marrow HSPCs upon O⁶BG/BCNU treatment were observed. A mild decrease in WBCs, specifically lymphocyte counts, was transient. Three to four cycles of low-dose treatment with O⁶BG, an inhibitor of DNA repair processes, and BCNU, an alkylating agent, resulting in survival of in vivo selected HSPCs could, theoretically, trigger mutations and tumorigenesis. Arguing against this risk are long-term follow-up studies in monkeys and dogs that received such treatment and did not suggest signs of carcinogenesis (Beard et al., J Clin Invest. 120(7):2345-2354, 2010, Radke et al., Sci Transl Med. 9(414):eaan1145, 2017, Beard et al., Blood. 113(21):5094-5103, 2009). In an attempt to assess this risk in HSPCs, an in vitro study was performed with CD34+ cells transduced with an MGMT^{P140K}-expressing HDAd vector and subjected to O⁶BG/BCNU treatment at a dose that killed 98% of cells that were not protected by MGMT^{P140K} expression (FIGS. 22A-22C). At day 14 after drug exposure, Illumina whole exome sequencing of CD34+ cells without treatment and cells that survived the treatment was performed, with the result shown in the following tables. Whole exome sequencing of CD34+ cells that survived drug treatment vs untreated CD34+ cells. Sample sequences were compared to a Homo sapiens reference genome (UCSC hg19).

### Sample #1: Untreated CD34+ Cells

| Total | Percent | Targeted | Read | Padded Target | Padded Read |
|---|---|---|---|---|---|
| Aligned reads | Aligned Reads | Aligned Reads | Enrichment | aligned Reads | Enrichment |
| 46,870,836 | 80.51% | 38,437,631 | 82.01% | 40,158,769 | 85.68% |
| Total | Percent | Targeted | Base | Padded Target | Padded Base |
| Aligned Bases | Aligned Bases | Aligned Bases | Enrichment | aligned Bases | Enrichment |
| 6,544,191,633 | 75.45% | 4,308,625,487 | 65.64% | 5,541,019,474 | 84.67% |

### Sample #2: Selected CD34+ Cells

| Total | Percent | Targeted | Read | Padded Target | Padded Read |
|---|---|---|---|---|---|
| Aligned reads | Aligned Reads | Aligned Reads | Enrichment | aligned Reads | Enrichment |
| 47,858,908 | 81.07% | 39,945,698 | 81.38% | 40,463,838 | 84.555 |
| Total | Percent | Targeted | Base | Padded Target | Padded Base |
| Aligned Bases | Aligned Bases | Aligned Bases | Enrichment | aligned Bases | Enrichment |
| 6,590,512,869 | 74.93% | 4,339,416,710 | 65.84% | 5,523,089,486 | 83.80% |

Using Sorting Intolerant from Tolerant (SIFT; available online at uswest.ensemble.org) as a filter that predicts whether an amino acid substitution affects protein function, 126 de novo mutations per 47,858,908 sequenced base pairs in the treated sample (2.63×10⁻⁶ mutations per base pair) were identified. Using ClinVar as a filter, six mutations with potential pathological effects were found. Table 11 summarizes on which chromosome unique mutations were found:

TABLE 13
Number of unique mutations

| | |
|---|---|
| Chromosome 1 | 793 |
| Chromosome 2 | 502 |
| Chromosome 3 | 369 |
| Chromosome 4 | 243 |
| Chromosome 5 | 253 |
| Chromosome 6 | 341 |
| Chromosome 7 | 383 |
| Chromosome 8 | 241 |
| Chromosome 9 | 312 |
| Chromosome 10 | 269 |
| Chromosome 11 | 536 |
| Chromosome 12 | 362 |
| Chromosome 13 | 94 |
| Chromosome 14 | 252 |
| Chromosome 15 | 271 |
| Chromosome 16 | 475 |
| Chromosome 17 | 527 |
| Chromosome 18 | 94 |
| Chromosome 19 | 755 |
| Chromosome 20 | 283 |
| Chromosome 21 | 92 |
| Chromosome 22 | 276 |
| Chromosome M | 0 |
| Chromosome X | 351 |
| Chromosome Y | 6 |
| Total | 8080 |

The finding that O⁶BG/BCNU treatment causes mutations is not unexpected; however, the consequences of the exome sequencing data are unclear. Loss-of-function variants are common in the human population. A recent analysis by the Exome Aggregation Consortium identified 3,230 genes with loss-of-function mutations, with 72% of these variants having no currently established human disease phenotype (Lek et al., Nature. 536(7616):285-291, 2016).

The HDAd-SB vector that carries SB100X transposase and Flpe recombinases gene does not integrate and is lost during cell division (Li et al., Mol Ther Methods Clin Dev. 9:142-152, 2018). In agreement with previously published data (Li et al., Mol Ther Methods Clin Dev. 9:142-152, 2018), neither integrated nor episomal HDAd-SB vector was detectable by qPCR at the end of the studies in bone marrow Lin− cells. SB100X transposase mediates random transgene integration without a preference for integration into or near genes (Richter et al., Blood. 128(18):2206-2217, 2016, Zhang et al., PLoS One. 8(10):e75344, 2013). This random pattern is maintained after in vivo selection without the emergence of dominant integration sites/clones. Theoretically, random integration is relatively safer than preferential integration into active genes, which occurs during lentivirus or AAV vector transduction (Deyle et al., Curr Opin Mol Ther. 11(4):442-447, 2009, Bartholomae et al., Mol Ther. 19(4):703-710, 2011, Schroder et al., Ce//. 110(4):521-529, 2002). Notably, in a SIN-LV-based clinical trial for β-thalassemia, integration into an intron of the HMGA2 proto-oncogene triggered a benign clonal dominance in one patient (Cavazzana-Calvo et al., Nature. 467(7313):318-322, 2010).

To reduce the risk of potential tumorigenicity from a combined effect of SB100X-mediated random transgene integration and treatment with mutagenic selection drugs, a vector system was designed to eliminate the first risk factor. It mediated targeted γ-globin integration into a chromosomal safe harbor site and resulted in stable γ-globin marking in more than 70% of RBCs in mice (Li et al., 21st Annual American Society of Gene and Cell Therapy Meeting. Abstract 972).

The safety of this approach may be first clearly documented in long-term studies in nonhuman primates. In this context it is notable that macaque and baboon bone marrow CD34+ cells are as efficiently transduced by Ad5/35 vectors as human CD34+ cells (Tuve et al., J Virol. 80(24):12109-12120, 2006), and direct in vivo transduction of mobilized CD34+ cells by an integrating HDAd5/35++vector expressing GFP in macaques was demonstrated (Harworth et al., 21st Annual American Society of Gene and Cell Therapy Meeting. Abstract 995).

Toward the clinical translation of the approach. Production of HDAd5/35++ vectors routinely yields 5×10¹² viral particles (vp) per liter spinner culture. cGMP-grade HDAd production for Flexion's FX201 vector is established. Protocols for the pharmacological control of innate immune reaction to intravenously injected virus are more developed for humans than for mice and are currently practiced in clinical trials with intravenously injected high-dose rAAV vectors. However, the majority of humans have neutralizing serum antibodies directed against Ad5 capsid proteins, which will block in vivo transduction with HDAd5/35 vectors, i.e., vectors that contain Ad5 capsid proteins and chimeric Ad35 fibers. An alternative described in this disclosure includes vectors derived from Ad35. Ad35 is one of the rarest of the 57 known human serotypes, with a seroprevalence of less than 7% and no cross-reactivity with Ad5 (Vogels et al., J Virol. 77(15):8263-8271, 2003, Abbink et al., J Virol. 81(9):4654-4663, 2007, Kostense et al., AIDS. 18(8):1213-1216, 2004, Flomenberg et al., J Infect Dis. 155(6):1127-1134, 1987, Barouch et al., Vaccine. 29(32):5203-5209, 2011). Ad35 is less immunogenic than Ad5 (Johnson et al., J Immunol. 188(12):6109-6118, 2012), which is, in part, due to attenuation of T cell activation by the Ad35 fiber knob (Adams et al., J Gen Virol. 93(pt 6):1339-1344, 2012. Adams et al., Proc Natl Acad Sci USA 108(18):7499-7504, 2011, Shoji et al., PLoS One. 7(1):e30302, 2012). After intravenous injection, there is only minimal transduction (only detectable by PCR) of tissues, including the liver, in human CD46-transgenic mice (Sakurai et al., Gene Ther. 13(14):1118-1126, 2006, Sakurai et al., Mol Ther. 16(4):726-733, 2008) and nonhuman primates (Sakurai et al., Mol Ther. 16(4):726-733, 2008). First-generation Ad35 vectors have been used clinically for vaccination purposes (Baden et al., Ann Intern Med. 164(5):313-322, 2016, Kazmin et al., Proc Natl Acad Sci USA 114(9):2425-2430, 2017). For upcoming studies in humans, vectors will be generated based on HDAd35++ for in vivo HSPC gene therapy.

In summary, this provides an alternative to traditional lentivirus vector ex vivo gene therapy for thalassemia, which may simplify the therapy and, theoretically, make it accessible to resource-poor regions where thalassemia major is endemic and HSPC transplantation not feasible.

### Example 2. In Vivo Hematopoietic Stem Cell Gene Therapy of Murine Thalassemia Using a 29 kb β-Globin Locus Control Region

Example 1 describes significant advances in the ability to drive γ-globin gene expression in in vivo modified HSPC. It also states that to increase the level of γ-globin expression further, a longer version (e.g., 26.1 kb) of the β-globin LCR might be used to drive γ-globin expression. This Example provides the results of that follow-up analysis.

As described herein, hematopoietic stem/progenitor cell (HSPC) mobilization followed by intravenous injection of integrating, helper-depending adenovirus HDAd5/35++ vectors resulted in efficient transduction of long-term repopulating cells and disease amelioration in mouse models after in vivo selection of transduced HSPCs. Acute innate toxicity associated with HDAd5/35++ injection was controlled by appropriate prophylaxis making this approach feasible for clinical translation. This technically can be used as a simple in vivo HSPC transduction approach for gene therapy of thalassemia major or Sickle Cell Disease. A cure of these diseases requires high expression levels of the therapeutic protein ( [symbol] - or β-globin), which is difficult to achieve with lentivirus vectors due to their genome size limitation not allowing larger regulatory elements to be accommodated. This example capitalizes on the 35 kb insert capacity of HDAd5/35++ vectors to demonstrate that transcriptional regulatory regions of the β-globin locus with a total length of 29 kb can efficiently be transferred into HSPCs. The in vivo HSPC transduction resulted in stable [symbol] -globin levels in erythroid cells that conferred a complete cure of murine thalassemia intermedia. Notably, this was achieved with a minimal in vivo HSPC selection regimen. This study demonstrates that HDAd5/35++ vectors that incorporate large regulatory regions can address challenges in gene therapy of diseases that require high-level transgene expression.

Introduction. For gene therapy of hemoglobinopathies such as thalassemia major and Sickle Cell Anemia to be successful, the transferred gene is preferably expressed in erythroid cells at high levels, without position effects of integration and transcriptional silencing. The β-globin locus control region (LCR) is thought to be beneficial in such use. For gene therapy applications, a β-globin LCR containing HSI to HS5 has been shown to confer high-level expression upon cis-linked genes in transgenic mice (Grosveld et al., Cell 51: 975-985, 1987). However, this version of the LCR is too large to be used in lentivirus vectors (insert capacity 8 kb) and, therefore truncated “mini” or “micro” LCR versions have been developed. For example, in ongoing clinical trials in thalassemia patients a lentivirus containing a 2.7 kb mini-LCR (covering HS2-HS4) and a 266 bp β-globin promoter is being used (Negre et al., Curr Gene Ther 15:64-81, 2015). In Example 1, a 5.9 kb β-globin LCR version was employed that contained HS1 to HS4 and the β-globin promoter for expression of γ-globin in CD46 transgenic mice or CD46/Hbb^{th3} thalassemic mice (Wang et al., J Clin Invest 129:598-615, 2019). With the in vivo HSPC transduction/selection approach, γ-globin marking was achieved in nearly 100% of peripheral blood erythrocytes, while the level of γ-globin expression was 10-15% of that of adult mouse α-globin with an average integrated vector copy number (VCN) of 2-3 copies per cell.

For a complete cure of β₀/β₀ thalassemia or Sickle Cell Anemia, it is generally thought that a therapeutic globin (either γ- or β-globin) expression level of 20% in erythroid cells is required (Fitzhugh et al., Blood 130:1946-1948, 2017). One way to reach this level is by increasing the VCN by improving HSPC transduction or increasing the vector dose. Such approaches, however, have historically been observed in other contexts to increase the risk of toxicity, at least in part due to random integration pattern of utilized vector systems. In this Example, stronger transcriptional elements, namely a longer LCR version, were utilized to increase γ-globin expression in RBCs after in vivo HSPC transduction of CD46-transgenic mice.

A novel in vivo HSPC transduction approach that does not require leukapheresis, myeloablation, and HSPC transplantation is provided (Richter et al., Blood, 128:2206-2217, 2016). The approach involves a new vector platform suitable for in vivo HSPC transduction, i.e. helper-dependent, capsid-modified adenovirus vectors (HDAd5/35++). Features of these vectors include CD46-affinity enhanced fibers that allow for efficient transduction of primitive HSCs while avoiding infection of non-hematopoietic tissues after i.v. injection and an insert capacity of up to 30 kb. Due to limited accessibility, HSPCs localized in the bone marrow cannot be transduced by intravenously injected vectors, including HDAd5/35++ vectors, even when the vector targets receptors that are present on bone marrow cells (Ni et al., Hum Gene Ther, 16: 664-677, 2005 and Ni et al., Cancer Gene Ther, 13:1072-1081, 2006). A combination of granulocyte-colony-stimulating factor (G-CSF) and the CXCR4 antagonists AMD3100 (MOZOBIL™, PLERIXA™) has been shown to efficiently mobilize primitive progenitor cells in animal models and in humans (Fruehauf et al., Cytotherapy, 11: 992-1001, 2009 and Yannaki et al., Hum Gene Ther, 24: 852-860, 2013). G-CSF/AMD3100 was used to mobilize HSPCs from the bone marrow into the peripheral blood stream followed by an intravenous injection of HDAd5/35++ vectors. This was shown previously in human CD46 transgenic mice (Richter et al., Blood, 128: 2206-2217, 2016; Li et al., Mol Ther Methods Clin Dev, 9: 390-401, 2018; Li et al., Blood, 131: 2915-2928. 2018; Wang et al., J Clin Invest, 129: 598-615. 2019; Wang et al., Blood Adv, 3: 2883-2894, 2019; and Wang et al., Mol Ther Methods Clin Dev, 8: 52-64, 2018), humanized mice (Richter et al., Blood, 128: 2206-2217, 2016) and rhesus macaques (Harworth et al., ASCGT 21th Annual meeting, 2018, DOI: 10.1016/j.ymthe.2018.05.001). HSPCs transduced in the periphery home back to the bone marrow where they persist long-term. Without a proliferative advantage, in vivo transduced HSPCs do not efficiently exit the bone marrow and contribute to downstream differentiation. Short-term treatment of animals with O⁶BG/BCNU provides a proliferation stimulus to mgmt^{P140K} gene-modified HSPCs and subsequent stable transgene expression in >80% of peripheral blood cells (Wang et al., Mol Ther Methods Clin Dev, 8: 52-64, 2018).

HD-Ad5/35++ genomes do not integrate into the host cell genome and are lost upon cell division. For gene therapy purposes and to trace in vivo transduced HSPCs long-term, HD-Ad5/35++ vectors were modified to allow for transgene integration. This was done by incorporating a hyperactive Sleeping Beauty transposase system (SB100) (Zhang et al., PLoS One, 8: e75344, 2013; Hausl et al., Mol Ther, 18: 1896-1906, 2010; and Yant et al., Nat Biotechnol, 20: 999-1005, 2002). The transposase, co-expressed in trans from a second vector, recognizes specific DNA sequences (inverted repeats; “IRs”) flanking the transgene cassette and triggers the integration into TA dinucleotides of the chromosomal DNA. Unlike retrovirus integration, SB100x-mediated integration does not depend on the transcriptional status of the targeted genes (Yant et al., Mol Cell Biol, 25: 2085-2094, 2005). Several studies have demonstrated SB100x-mediated transgene integration is random and has not been associated with the activation of proto-oncogenes (Richter et al., Blood, 128: 2206-2217, 2016; Wang et al., Mol Ther Methods Clin Dev, 8: 52-64, 2018; Zhang et al., PLoS One, 8: e75344, 2013; Hausl et al., Mol Ther, 18: 1896-1906, 2010; and Yant et al., Nat Biotechnol, 20: 999-1005, 2002). An advantage of the SB100x-based integration system is that it does not depend on an efficient homologous DNA repair machinery of the cell. The latter is critical in HSPCs, which show low activity of DNA repair and recombination enzymes (Beerman et al., Cell Stem Cell, 15: 37-50, 2014). It was demonstrated that in vivo HSC co-infection with a HDAd35++-transposon vector and a SB100x/Flpe expressing vector in CD46-transgenic mice (Richter et al., Blood, 128: 2206-2217, 2016; Wang et al., J Clin Invest, 129: 598-615. 2019; Li et al., Mol Ther, 27: 2195-2212, 2019; Li et al., Mol Ther Methods Clin Dev, 9: 142-152, 2018; and Wang et al., J Virol, 79: 10999-11013, 2005) and human CD34+ cells (Li et al., Mol Ther, 27: 2195-2212, 2019) resulted in random transgene integration of 2 transgene copies/cell without a preference for genes.

The human genome is organized in a 3-D structure with long-range interactions between regulatory regions (i.e. transcription factor binding sites) usually through loop forming. Most of these interactions occur in the context of topologically associating domains (TADs). TADs are considered functional units of chromosome organization in which enhancers interact with other regulatory regions to control transcription. TAD/LCR border insulation is thought to restrict the search space of enhancers and promoters and to prevent unwanted regulatory contacts to be formed. Boundaries at both sides of these domains are conserved between different mammalian cell types and even across species.

Currently used lentivirus and rAAV gene transfer vectors can accommodate only small enhancers/promoters, often resulting in suboptimal level and tissue specificity of transgene expression, transgene silencing, and unintentional interactions with regulatory regions surrounding the vector integration site. In the worst-case scenario, the latter can lead to the activation of proto-oncogenes.

To increase the safety and efficacy of gene therapy, TADs should be used for gene addition strategies. The median size of TAD is 880 kb. With further advancement of high-throughput chromosome conformation capture (3C) assay and its subsequent 4C, 5C and H-C protocols as well as fiber-Seq assays, the interrogation of regulatory genome will progress at a rapid speed and, for gene therapy purposes, could deliver TADs that contain only critical core elements. The β-globin Locus Control Region (LCR) fails under the definition of a TAD.

Capsid-modified HDAd5/35++ vectors have been used for in vivo HSPC gene therapy (Li & Lieber, FEBS Lett. 593(24):3623-48, 2019; Richter et al., Blood. 128(18):2206-17, 2016). The approach involves the mobilization of HSPCs from the bone marrow, and while they circulate at high numbers in the periphery, HDAd5/35++ vectors are injected intravenously. These vectors target CD46, a receptor that is expressed on primitive HSPCs (Richter et al., Blood. 128(18):2206-17, 2016). Transduced HSPCs return to the bone marrow where they persist long-term. Random integration is mediated by an activity-enhanced Sleeping Beauty transposase (SB100x) (Boehme et al., Mol Ther Nucleic Acids. 5(7):e337, 2016). Targeted integration can be achieved via homology dependent DNA repair (Li et al., Mol Ther. 27(12):2195-212, 2019). This approach resulted in an amelioration of murine thalassemia intermedia (Wang et al., J Clin Invest. 129(2):598-615, 2019), the correction of murine hemophilia (Wang et al., Blood Adv. 3(19):2883-94, 2019), and the reversion of spontaneous cancer (Li et al., Cancer Res. 80(3):549-560, 2019). First data in non-human primates show that the in vivo HSPC gene therapy approach is safe when combined with glucocorticoid, IL6- and IL1β-receptor antagonist pretreatment to suppress innate immune responses after intravenous HDAd5/35++injection (Li et al., 23rd Annual ASGCT meeting. 2020; abstract #546). The intravenous injection of HDAd5/35++ vectors did not result in transgene expression in tissues other than the mobilized HSPCs and PBMCs in CD46tg mice at day 3 after injection (Richter et al., Blood. 128(18):2206-17, 2016; Wang et al., J Clin Invest. 129(2):598-615, 2019). This was recently confirmed in non-human primates. A potential explanation for this tropism is that CD46 receptor density and accessibility is not sufficiently high in non-hematopoietic tissues to allow for efficient viral transduction (Richter et al., Blood. 128(18):2206-17, 2016; Ni et al., Hum Gene Ther. 16(6):664-77, 2005).

In a previous study with HDAd5/35++ vectors, a 4.3 kb HSI-HS4 mini-LCR (β-globin locus control region) was used in combination with a 0.66 kb β-globin promoter to drive human γ-globin expression after in vivo HSPC transduction (Wang et al, J Clin Invest. 129(2):598-615, 2019; Ong et al., Exp Hematol. 34(6):713-20, 2006). In Hbb^{th3}/CD46+/+thalassemic mice, stable (8+months) γ-globin marking was achieved in nearly 100% of peripheral blood erythrocytes and near complete phenotypic correction (Wang et al., J Clin Invest. 129(2):598-615, 2019). However, the level of γ-globin expression was only 10-15% of that of adult mouse α-globin with an average integrated vector copy number (VCN) of 2 copies per cell, thus rendering the clinical translation of the approach to thalassemia major or SCD particularly challenging. Here, the large capacity of HDAd5/35++ vectors was exploited by incorporating β-globin TAD core elements including a γ-globin expression cassette with a length of 29 kb to achieve complete phenotypic correction

In this context, another intention was to demonstrate that the SB100x system can mediate the efficient integration of a 32.4 kb transposon. From studies with plasmid-based SB systems it was thought that the SB integration activity negatively correlated with the length of the transposon (Li et al., Mol Ther Methods Clin Dev. 9:142-52, 2018; Karsi et al., Mar Biotechnol (NY). 3(3):241-5, 2001). Taking this into consideration, the first SB-based HDAd vectors developed by the Kay and Ehrhardt groups carried relatively small (4 kb-6 kb) transposons (Turchiano et al., PLoS One. 9(11):e112712, 2014; Yant et al., Nat Biotechnol. 20(10):999-1005, 2002).

Recently, using HDAd5/35++ vectors, efficient SB100x-mediated integration of 10.8 kb (Wang et al., Blood Adv. 3(19):2883-94, 2019) and 11.8 kb (Wang et al., J Clin Invest. 129(2):598-615, 2019; Ong et al., Exp Hematol. 34(6):713-20, 2006) transposons in HSPC was demonstrated after ex vivo or in vivo HSPC transduction. This example provides proof that the HDAd5/35++-based SB100x vector system can integrate a 32.4 kb transposon.

Overall, these in vivo studies in normal and thalassemic mice as well as in vitro studies with human CD34+ cells indicate that the described long-LCR containing HDAd5/35++ vector can be an efficient therapeutic tool for the treatment of hemoglobinopathies.

Materials and Methods.

Component Positions: HS5→HS1 (21.5 kb): Chr11, 5292319→5270789; p-promoter: chr11, 5228631→5227023; and 3′HS1: Chr11, 5206867→5203839.

HDAd vectors: The generation of HDAd-SB and HDAd-short-LCR vector has been described previously (Richter et al., Blood 128: 2206-2217, 2016; Ong et al., Exp Hematol 34(6):713-20, 2006). For the generation of the HDAd-long-LCR vector, corresponding shuttle plasmids were based on the cosmid vector pWE15 (Stratagene, La Jolla, Calif.). pWE.Ad5-SB-mgmt contains the Ad5 5′ITR (nucleotides 1 through 436) and 3′ITR (nucleotides 35741 through 35938), the human EF1α promoter-mgmt^{P140K}-SV40 pA-cHS4 cassette derived from pBS-pLCR-γ-globin-mgmt (Wang et al., J Clin Invest 129: 598-615, 2019), SB100x-specific IR/DR sites and FRT sites. The GFP-BGHpA fragment in the pAd.LCR-β-GFP (containing a 21.5-kb human β-globin LCR (Hudecek et al., Crit Rev Biochem Mol Biol 52(4):355-380, 2017) was replaced by the human γ-globin gene and its 3′UTR region (Chr 11:5,247,139→5,249,804) (pAd-long-LCR-β-γ-globin). The plasmid pAd-long-LCR-β-γ-globin contains a 21.5-kb human β-globin LCR and 3.0-kb human β-globin 3′HS1. The 28.9-kb fragment containing LCR-β-γ-globin-3′HS1 was inserted downstream of the cassette of EF1α-mgmt-SV40 pA-cHS4 into pWE.Ad5-SB-mgmt (pWE.Ad5-SB-long-LCR-γ-globin/mgmt). The complete long-LCR-γ-globin/mgmt cassette was flanked by SB100x-specific IR/DR sites and FRT sites. The resulting plasmids were packaged into phages using Gigapack III Plus Packaging Extract (Stratagene, La Jolla, Calif.) and propagated. To generate the HD-Ad-long-LCR-γ-globin/mgmt virus, the viral genomes were released by I-Ceul digestion from the plasmid for rescue in 116 cells. There are two known variants of the HBG1 gene in the human population with a single amino acid variation (76-Isoleucine or 76-Threonine). The 76-Ile HBG1 variant was used which has a range in frequency from 13% in Europeans to 73% in East Asians.

To generate HDAd viruses, the viral genomes were released by Fsel digestion from the plasmid for rescue in 116 cells (Palmer et al., Mol Ther 8: 846-852, 2003) with Ad5/35++-Acr helper virus. This helper virus is a derivative of AdNG163-5/35++, an Ad5/35++ helper vector containing chimeric fibers composed of the Ad5 fiber tail, the Ad35 fiber shaft, and the affinity-enhanced Ad35++fiber knob (Richter et al., Blood 128: 2206-2217, 2016). A human codon-optimized AcrIIA4-T2A-AcrIIA2 sequence that was recently shown to inhibit SpCas9 activity was synthesized (Yang et al., Proc Natl Acad Sci USA. 92(25):11608-12, 1995) and cloned into a shuttle plasmid pBS-CMV-pA (pBS-CMV-Acr-pA). Subsequently, the 2.0-kb CMV-Acr-pA cassette was amplified from pBS-CMV-Acr-pA and inserted into the Swal sites of pNG163-2-5/35++ (Richter et al., Blood 128: 2206-2217, 2016) by In-Fusion HD cloning kit (Takara). The viral genome was then released by PacI digestion and the Ad5/35++-Acr helper virus was rescued and propagated in 293 cells (HEK293). The generation of HDAd-SB has been described previously (Richter et al., Blood 128: 2206-2217, 2016). Helper virus contamination levels were below 0.05%. All preparations were free of bacterial endotoxin.

CD34+ cell culture: CD34+ cells from G-CSF-mobilized adult donors were recovered from frozen stocks and incubated overnight in Iscove's modified Dulbecco's medium (IMDM) supplemented with 10% heat-inactivated FCS, 1% BSA 0.1 mmol/l 2-mercaptoethanol, 4 mmol/l glutamine and penicillin/streptomycin, Flt3 ligand (Flt3L, 25 ng/mi), interleukin 3 (10 ng/mi), thrombopoietin (TPO) (2 ng/mi), and stem cell factor (SCF) (25 ng/mi). Flow cytometry demonstrated that >98% of cells were CD34+. Cytokines and growth factors were from Peprotech (Rocky Hill, N.J.). CD34+ cells were transduced with virus in low attachment 12 well plates.

Erythroid in vitro differentiation: Differentiation of human HSPCs into erythroid cells were carried out based on the protocol described in Douay et al. (Methods Mol Bio/482: 127-140, 2009). In brief, in step 1, cells at a density of 10⁴ cells/ml were incubated for 7 days in IMDM supplemented with 5% human plasma, 2 IU/ml heparin, 10 μg/ml insulin, 330 μg/ml transferrin, 1 μM hydrocortisone, 100 ng/ml SCF, 5 ng/ml IL-3, 3 U/ml erythropoietin (Epo), glutamine, and Pen-Strep. In step 2, cells at a density of 1×10⁵ cells/ml were incubated for 3 days in IMDM supplemented with 5% human plasma, 2 IU/ml heparin, 10 μg/ml insulin, 330 μg/ml transferrin, 100 ng/ml SCF, 3 U/ml Epo, glutamine, and Pen/Strep. In step 3, cells at a density of 1×10⁶ cells/ml cells were incubated for 12 days in IMDM supplemented with 5% human plasma, 2 IU/ml heparin, 10 μg/ml insulin, 330 μg/mi transferrin, 3 U/ml Epo, glutamine, and Pen/Strep.

In vitro selection of transduced CD34+ cells: Transduced CD34+ cells were selected with O⁶BG/BCNU on day 5 in step 1 of the in vitro differentiation protocol. Briefly, CD34+ cells were incubated with 50 μM O⁶13 G for one hour and then incubated with 35 μM BCNU for another two hours, cells were then washed twice and resuspended in fresh step 1 medium.

Lin⁻ cell culture: Lineage negative cells were isolated form total mouse bone marrow cells by MACS using the Lineage Cell Depletion kit from Miltenyi Biotech (Bergisch Gladbach, Germany). Lin⁻ cells were cultured in IMDM supplemented with 10% FCS, 10% BSA, Pen-Strep, glutamine, 10 ng/ml human TPO, 20 ng/ml mouse SCF and 20 ng/ml human Fit-3L.

Globin HPLC: Individual globin chain levels were quantified on a Shimadzu Prominence instrument with an SPD-10AV diode array detector and an LC-10AT binary pump (Shimadzu, Kyoto, Japan). A 40%-60% gradient mixture of 0.1% trifluoroacetic acid in water/acetonitrile was applied at a rate of 1 mL/min using a Vydac C4 reversed-phase column (Hichrom, UK).

Flow cytometry: Cells were resuspended at 1×10⁶ cells/100 μL in PBS supplemented with 1% FCS and incubated with FcR blocking reagent (Miltenyi Biotech, Auburn Calif.) for ten minutes on ice. Next the staining antibody solution was added in 100 μL per 10⁶ cells and incubated on ice for 30 minutes in the dark. After incubation, cells were washed once in FACS buffer (PBS, 1% FBS). The staining step was repeated with a secondary staining solution. After the wash, cells were resuspended in FACS buffer and analyzed using a LSRII flow cytometer (BD Biosciences, San Jose, Calif.). Debris was excluded using a forward scatter-area and sideward scatter-area gate. Single cells were then gated using a forward scatter-height and forward scatter-width gate. Flow cytometry data were then analyzed using FlowJo (version 10.0.8, FlowJo, LLC). For flow analysis of LSK cells, cells were stained with biotin-conjugated lineage detection cocktail (cat #: 130-092-613; Miltenyi Biotec, San Diego, Calif.) and antibodies against c-Kit (cat #:12-1171-83) and Sca-1 (cat #: 25-5981-82) as well as APC-conjugated streptavidin. Other antibodies from eBioscience (San Diego, Calif.) included anti-mouse LY-6A/E (Sca-1)-PE-Cyanine7 (clone D7), anti-mouse CD117 (c-Kit)-PE (clone 2B8), anti-mouse CD3-APC (clone 17A2; cat #:17-0032-82), anti-mouse CD19-PE-Cyanine7 (clone eBio1D3; cat #: 25-0193-82), and anti-mouse Ly-66 (Gr-1)-PE, (clone RB6-8C5; cat #: 12-5931-82). Anti-mouse Ter-119-APC (clone: Ter-119; cat #: 116211) was from Biolegend (San Diego, Calif.).

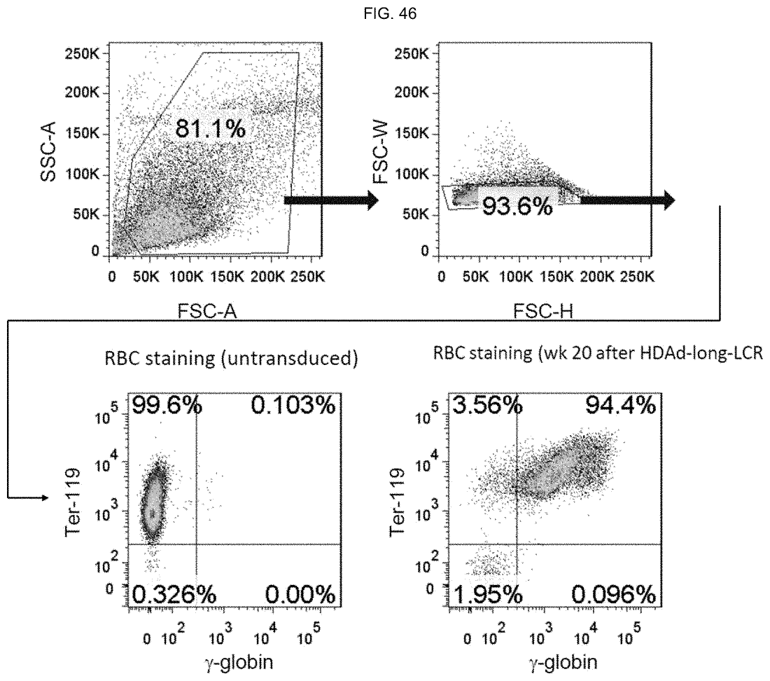

Intracellular flow cytometry detecting human γ-globin expression: The FIX & PERM™ (Nordic Immunological Laboratories, Susteren, Netherlands) cell permeabilization kit (Thermo Fisher Scientific, Waltham, Mass.) was used and the manufacture's protocol was followed. Briefly, 1×10⁶ cells were resuspended in 100 μl FACS buffer (PBS supplemented with 1% FCS), 100 μl of reagent A (fixation medium) was added and incubated for 2-3 minutes at room temperature, 1 ml pre-cooled absolute methanol was then added, mixed and incubated on ice in the dark for 10 minutes. The samples were then washed with FACS buffer and resuspended in 100 μl reagent B (permeabilization medium) and 0.3 μg hemoglobin γ antibody (Santa Cruz Biotechnology, Dallas, Tex., cat #sc-21756 PE), incubated for 30 minutes at room temperature. After the wash, cells were resuspended in FACS buffer and analyzed. Flow cytometry gating strategies are shown in FIG. 46.

Real-time reverse transcription PCR: Total RNA was extracted from 50-100 μl blood by using TRIzol™ reagent (Thermo Fisher Scientific) following the manufacture's phenol-chloroform extraction method. Quantitect reverse transcription kit (Qiagen) and power SYBR™ green PCR master mix (Thermo Fisher Scientific) were used. Real time quantitative PCR was performed on a StepOnePlus real-time PCR system (AB Applied Biosystems). The following primer pairs were used: mouse RPL10 (house-keeping) forward (SEQ ID NO: 189), and reverse (SEQ ID NO: 190); human γ-globin forward (SEQ ID NO: 191), and reverse (SEQ ID NO: 192); mouse β-major globin forward (SEQ ID NO: 193), and reverse (SEQ ID NO: 194), mouse a globin forward (SEQ ID NO: 212), and reverse (SEQ ID NO: 213).

Measurement of vector copy number: Total DNA from bone marrow cells was extracted using the Quick-DNA miniprep kit (Zymo Research). Viral DNA extracted from HDAd-short LCR-γ-globin/mgmt virus was serially diluted and used for a standard curve. qPCR was conducted in triplicate using the power SYBR Green PCR master mix on a StepOnePlus real-time PCR system (Applied Biosystems). 9.6 ng DNA (9600 μg/6 μg/cell=1600 cells) was used fora 10 μL reaction. The following primer pairs were used: human γ-globin forward (SEQ ID NO: 195), and reverse (SEQ ID NO: 196).

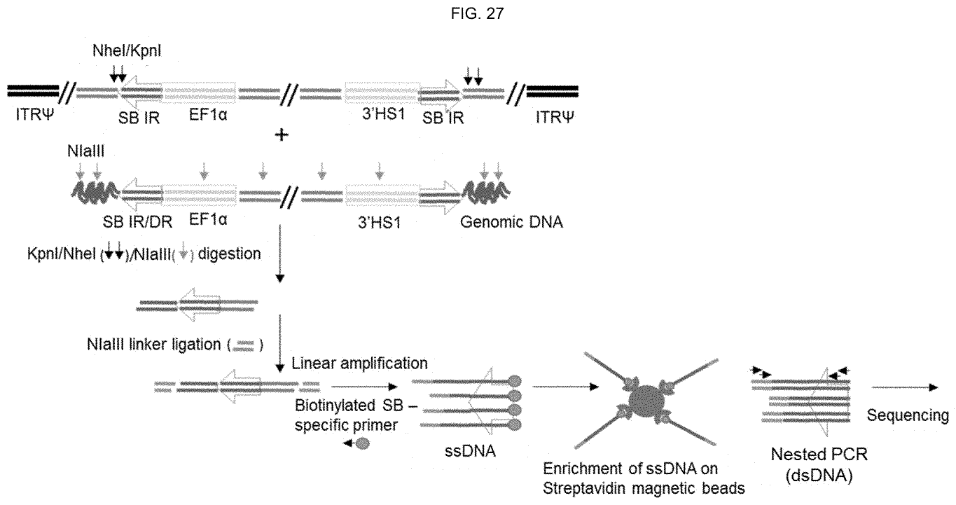

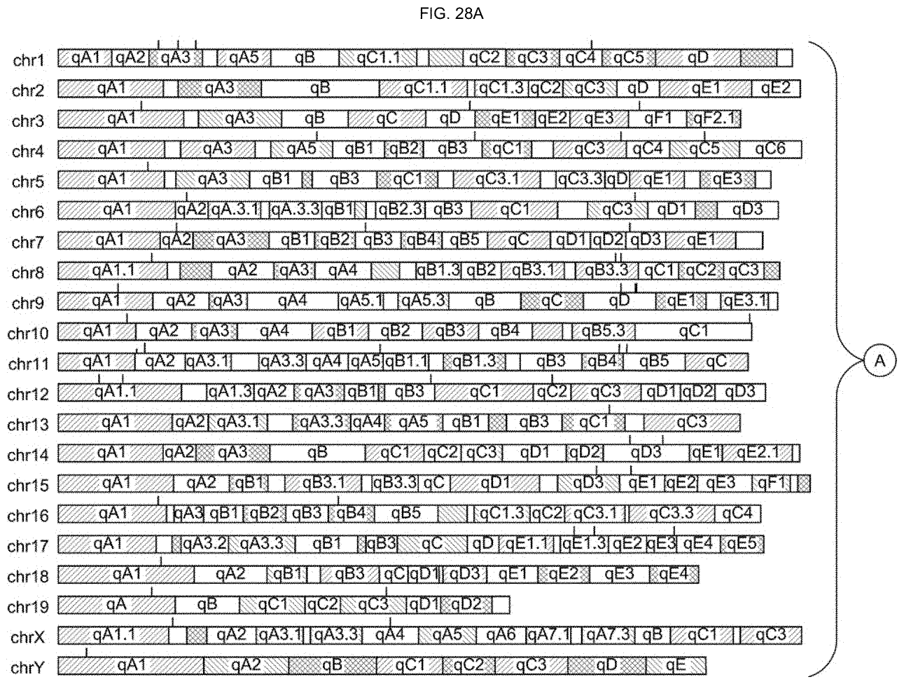

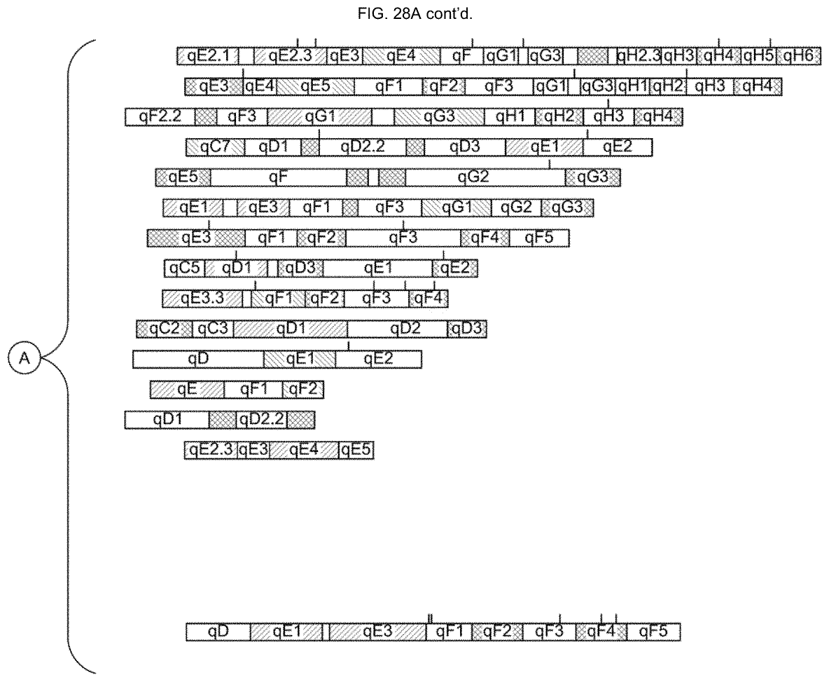

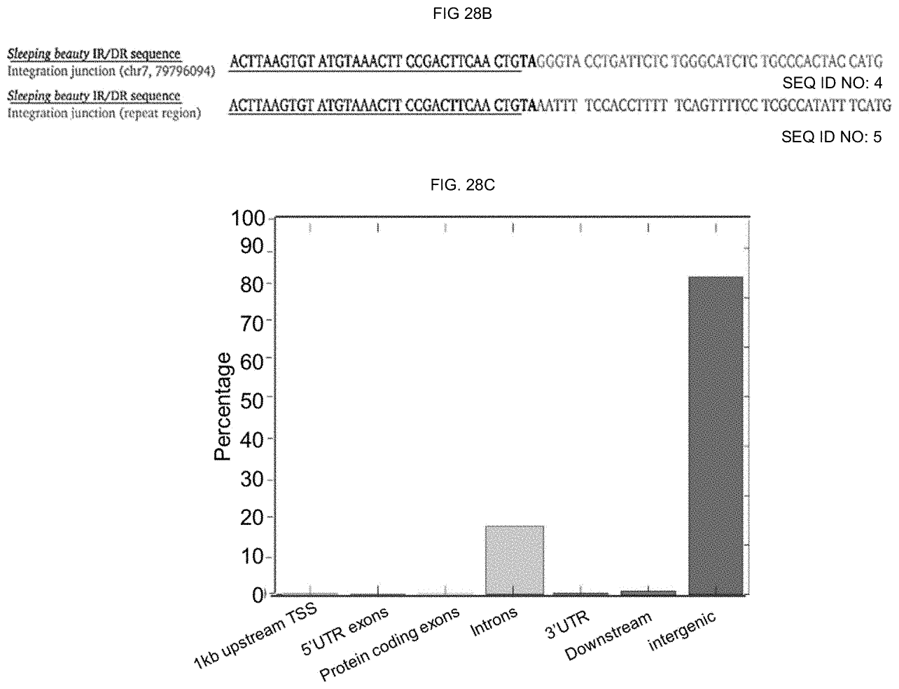

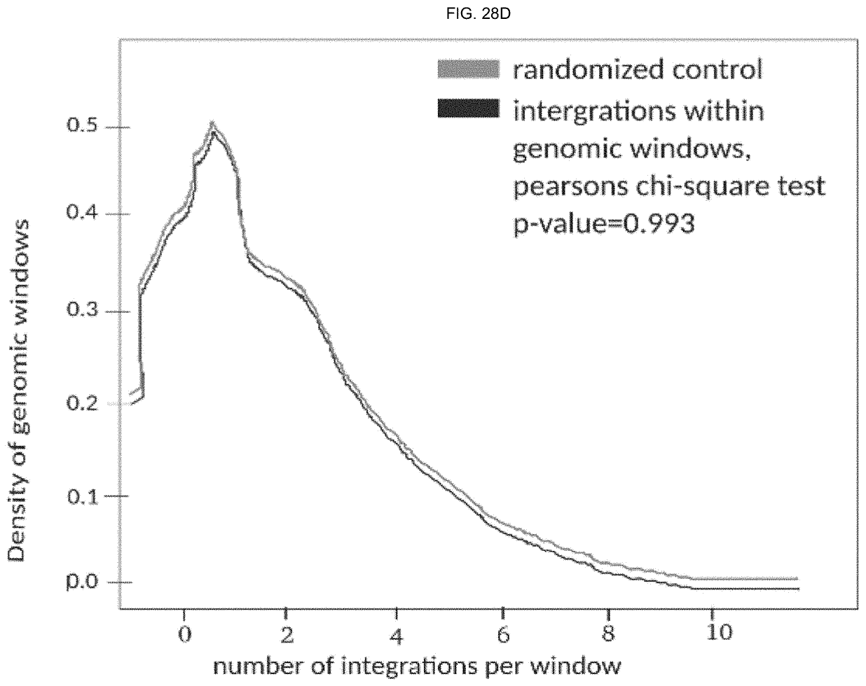

Integration site analysis. For a description of the procedure, see FIG. 27. The randomized data for FIG. 28D was created using a Poisson Regression Insertion Model (PRIM) to calculate the expected insertion rate for non-overlapping 20 kilobase windows along the length of each chromosome in the mouse reference genome (mm9). The PRIM algorithm generated a statistical model based on the number of TA dinucleotides within each window, the chromosome in which the window resides, and the total number of unique insertions. For each window, the expected number of insertions was calculated and compared to the observed number of insertions to produce a p-value. Bonferroni-correction was then applied to identify windows that showed enrichment for detection of inserted transposons. Random sequences from the reference genome containing TA were then generated, mapped using Bowtie2 and plotted against the real integration data. Calculations and plots were made using ggplot2 in R. figures were drawn using HOMER and ChlPseeker.

Integration site analysis (inverse PCR). Junctions in total bone marrow cells were analyzed by inverse PCR as described elsewhere with modifications (Hudecek et al., Crit Rev Biochem Mol Biol 52(4):355-80, 2017). Briefly, genomic DNA from bone marrow cells was isolated by Quick-DNA miniprep kit (Zymo Research) following the manufacturer's instructions. 5-10 μg of DNA was digested with SacI and re-ligated under conditions that promote intramolecular reaction. The ligation mixture was purified with phenol/chloroform extraction and ethanol precipitation and then used for nested PCR (30 cycles each) using KOD Hot Start DNA polymerase. The following primers were used: EF1α p1 forward (SEQ ID NO: 197) and reverse (SEQ ID NO: 198); EF1α p2 forward (SEQ ID NO: 199) and reverse (SEQ ID NO: 200); 3′HS1 p1 forward (SEQ ID NO: 201) and reverse (SEQ ID NO: 202); and 3′HS1 p2 forward (SEQ ID NO: 203) and reverse (SEQ ID NO: 204). In SEQ ID NOs: 197-204, the underlined bases are used for downstream cloning. PCR amplicons were gel purified, cloned, sequenced and aligned to identify the integration sites.

RNA-seq analysis was performed by Omega Bioservices (Norcross, Ga.). Data was analyzed by Rosalind (available online at rosalind.onramp.bio/), with a HyperScale architecture developed by OnRamp Biolnformatics, Inc. (San Diego, Calif.). Reads were trimmed using cutadapt. Quality scores were assessed using FastQC. Individual sample reads were quantified using HTseq4 and normalized via Relative Log Expression (RLE) using DESeq2 R library. DEseq2 was also used to calculate fold changes and p-values and perform optional covariate correction. Clustering of genes for the final heatmap of differentially expressed genes was done using the PAM (Partitioning Around Medoids) method using the fpc R library. Several database sources were referenced for enrichment analysis, including Interpro9, NCBI10, MSigDB11,12, REACTOMEI3, WikiPathways. Enrichment was calculated relative to a set of background genes relevant for the experiment.

The volcano plot was generated with a custom Python script that plots log-scale fold change versus p-values.

Animals:

Study approval: All experiments involving animals were conducted in accordance with the institutional guidelines set forth by the University of Washington. The University of Washington is an Association for the Assessment and Accreditation of Laboratory Animal Care International (AALAC)-accredited research institution and all live animal work conducted at this university is in accordance with the Office of Laboratory Animal Welfare (OLAVV) Public Health Assurance (PHS) policy, USDA Animal Welfare Act and Regulations, the Guide for the Care and Use of Laboratory Animals and the controlling Institutional Animal Care and Use Committee (IACUC) policies. The studies were approved by the University of Washington IACUC (Protocol No. 3108-01).

Ex vivo and in vivo HSPC transduction studies were performed with a C57Bl/6-based transgenic mouse model (hCD46tg) that contained the complete human CD46 locus. These mice express hCD46 in a pattern and ata level similar to humans (Wang et al., Mol Ther Methods Clin Dev. 8:52-64, 2018).

Breeding and screening of Hbb^{th3}/CD46+/+ mice: After three rounds of backcrossing, Hbbth³ mice homozygosity for CD46 was confirmed by PCR on gDNA (using CD46F-5′ (SEQ ID NO: 205) and CD46R primers (SEQ ID NO: 206) as well as by flow cytometry that allowed measuring CD46 MFI. The thalassemic phenotype of Hbb^{th3}/CD46+/+ mice was assessed by peripheral blood smears, after Giemsa/May-Grunwald staining, as described below.

Bone marrow Lin⁻ cell transplantation: Recipients were female C57BL/6 mice, 6-8 weeks old. On the day of transplantation, recipient mice were irradiated with 1000 Rad. Four hours after irradiation 1×10⁶ Lin⁻ cells were injected intravenously through the tail vein. This protocol was used for transplantation of ex vivo transduction Lin⁻ cells and for transplantation into secondary recipients.

HSPC mobilization and in vivo transduction: This procedure was described previously in Richter, et al., (2016) Blood 128: 2206-2217. HSPCs were mobilized in mice by s.c. injections of human recombinant G-CSF (5 μg/mouse/day, 4 days) (Amgen Thousand Oaks, Calif.) followed by an s.c. injection of AMD3100 (5 mg/kg) (Sigma-Aldrich) on day 5. In addition, animals received Dexamethasone (10 mg/kg) i.p. 16 h and 2 h before virus injection. Thirty and 60 minutes after AMD3100, animals were intravenously injected with HDAd vectors through the retro-orbital plexus with a dose of 4×10¹⁰ vp for each virus per injection. Four weeks later, in vivo selection of O⁶BG/BCNU was initiated.

Secondary bone marrow transplantation: Recipients were female C57BL/6 mice, 6-8 weeks old from the Jackson Laboratory. On the day of transplantation, recipient mice were irradiated with 1000 Rad. Bone marrow cells from in vivo transduced CD46tg mice were isolated aseptically and lineage-depleted cells were isolated using MACS. Four hours after irradiation cells were injected intravenously at 1×10⁶ cells per mouse. At week 20, secondary recipients were either sacrificed and CD46+ cells from blood, bone marrow and spleen were isolated by MACS or subjected to mobilization and in vivo transduction, as described above. All secondary recipients received immunosuppression starting at week 4.

Hematological analyses: Blood samples were collected into EDTA-coated tubes, and analysis was performed on a HemaVet 950FS (Drew Scientific).

Tissue analysis: Spleen and liver tissue sections of 2.5 μm thickness were fixed in 4% formaldehyde for at least 24 hours, dehydrated and embedded in paraffin. Staining with hematoxylin-eosin was used for histological evaluation of extramedullary hemopoiesis. Hemosiderin was detected in tissue sections by Perl's Prussian blue staining. Briefly, the tissue sections were treated with a mixture of equal volumes (2%) of potassium ferrocyanide and hydrochloric acid in distilled water and then counterstained with neutral red. To quantitate extracellular hemopoiesis and hemosiderosis, 10 random areas in 5 different tissue sections from at least 3 animals were evaluated by investigators that were blinded for the mouse groups. The spleen size was assessed as the ratio of spleen weight (mg)/body weight (g).

Blood analysis and bone marrow cytospins: Blood samples were collected into EDTA-coated tubes and analysis was performed on a HemaVet 950FS (Drew Scientific, Waterbury, Conn.). Peripheral blood smears and bone marrow cell cytospins were stained with Giemsa/May-Grunwald/Giemsa (Merck, Darmstadt, Germany) for 5 and 15 minutes, respectively. Reticulocytes were stained with Brilliant cresyl blue. The investigators who counted the reticulocytes on blood smears have been blinded to the sample group allocation. Only animal numbers appeared on the slides (5 slides per animal, 5 random 1 cm² sections).

Statistical analyses: Data are presented as means±standard error of the mean (SEM). For comparisons of multiple groups, one-way and two-way analysis of variance (ANOVA) with Bonferroni post-testing for multiple comparisons was employed. Differences between groups for one grouping variable were determined by the unpaired, two-tailed Student's t-test. For non-parametric analyses the Kruskal-Wallis test was used. Statistical analysis was performed using GraphPad Prism version 6.01 (GraphPad Software Inc., La Jolla, Calif.). *p≤0.05, **p≤00.0001. A P value less than 0.05 was considered significant.

Results.

As a model for the in vivo transduction studies with intravenously injected HDAd5/35++vectors, transgenic mice were used that contain the complete human CD46 locus and therefore express hCD46 in a pattern and at a level similar to humans (hCD46tg mice) (Kemper, et al., (2001) Clin Exp Immunol 124: 180-189).

HDAd5/35++ vector containing a long β-globin LCR. In the studies described in Example 1, a HDAd5/35++ vector (FIG. 23, “HDAd-short-LCR”) (Wang et al., J Clin Invest 129: 598-615, 2019) was used expressing γ-globin under the control of a 4.3 kb mini LCR (encompassing the core elements of HS1 to HS4 (Lisowski et al., Blood 110: 4175-4178, 2007)) linked to a 1.6 kb β-globin promoter (Wang et al., J Clin Invest 129: 598-615, 2019; Li, et al., ( ) Mol Ther Methods Clin Dev 9: 142-152, 2018). In the present Example, an HDAd5/35++ vector was constructed that contained the following elements to maximize γ-globin gene expression: i) a 21.5 kb LCR including the full-length HS5 to HS1 regions, ii) a 1.6 kb β-globin promoter, iii) a β-globin 3′UTR to stabilize γ-globin mRNA, and iv) a 3′ HS1 region. The vector was named HDAd-long-LCR (FIG. 23, “HDAd-long-LCR”). To mediate integration, the LCR-vectors are used in combination with a SB100x/Flpe expressing HDAd vectors (FIG. 23, “HDAd-SB”). The transposon vectors (HDAd-short-LCR and HDAd-long-LCR) contain inverted/direct repeat (IR/DRs) motifs, which are recognized by the SB100x transposase and frt sites that allow for circularization of the transgene cassette in the presence of Flpe recombinase. Both HDAd-short-LCR and HDAd-long-LCR also carried the gene for a mutant O⁶-methylguanine-DNA methyltransferase (mgmt^{P140K}) under control of the ubiquitously active EF1α promoter to allow for selection of stably transduced cells by low-dose O⁶BG/BCNU treatment (Hausl et al., B. Mol Ther 18(11):1896-906, 2010; Neff et al., J Clin Invest 112(10):1581-8, 2003).

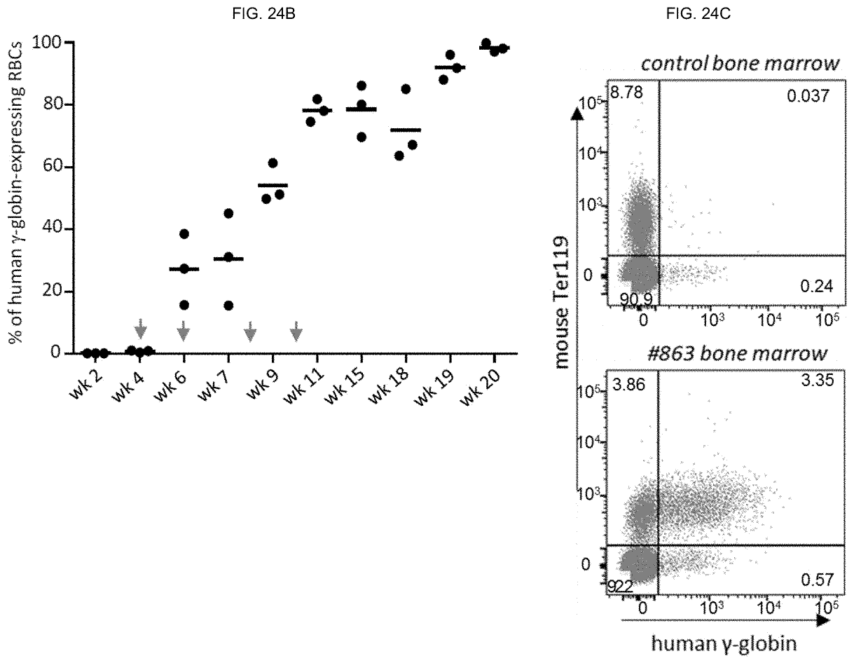

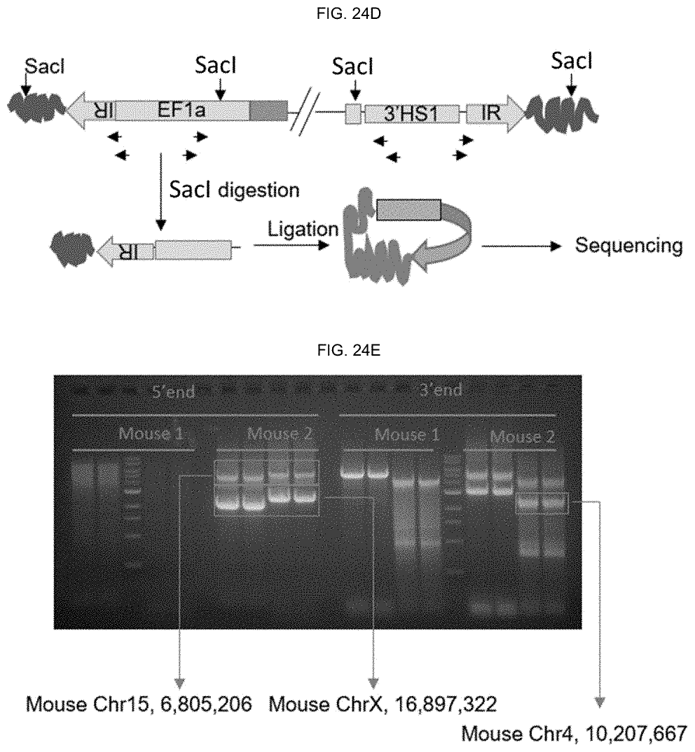

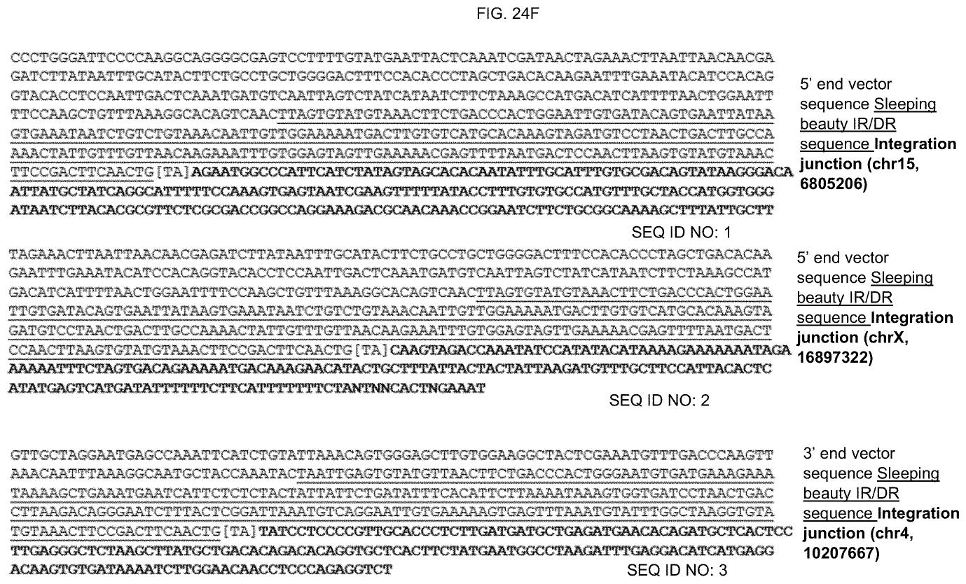

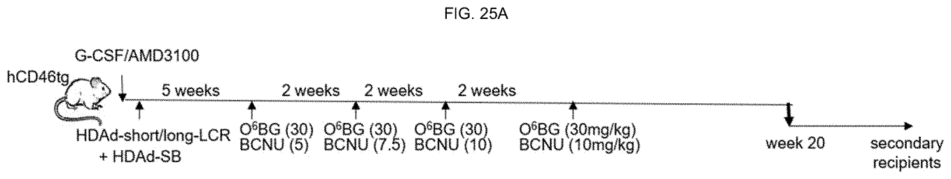

Ex vivo HSPC transduction/transplantation study. While in humans, CD46 is expressed on all nucleated cells, the corresponding orthologue in mice is present only in the testes. As a model for in vivo transduction studies with intravenously injected HDAd5/35++ vectors, transgenic mice that contained the complete human CD46 locus were used and therefore expressed hCD46 in a pattern and at a level similar to humans (CD46tg mice) (Wang et al., Mol Ther Methods Clin Dev 8:52-64, 2018). Because, a priori, it was not known whether SB100x can mediate the integration of the 32.4 kb transposon, ex vivo HSPC transduction studies were performed, in a setting where the HSPC transduction efficacy could be controlled. CD46tg mouse bone marrow lineage-negative (Lin⁻) cells, a cell fraction enriched for HSPCs were transduced ex vivo with HDAd-long-LCR+HDAd-SB (FIG. 24A). Ex vivo transduced cells were then transplanted into lethally irradiated C57Bl/6 mice. Engraftment rates at week 4 were >95% based on CD46-positive PBMCs. One month after transplantation, mice were subjected to four rounds of O⁶BG/BCNU treatment to selectively expand progenitors with integrated γ-globin/mgmt transgenes (FIG. 24A). With each round of in vivo selection, the percentage of γ-globin-positive peripheral red blood cells (RBCs) increased, reaching >95%, by the end of the study (FIG. 24B). At week 20, animals were sacrificed and bone marrow mononuclear cells (MNCs) were analyzed. The average VCN measured by qPCR was 2.8 copies per cell. γ-globin expression was detected by flow cytometry in 85.46(+/−5.9)% of erythroid Ter119⁺ cells and in 14.54(+/−2.3)% non-erythroid (Ter119⁻) bone marrow MNCs (FIG. 24C).

To demonstrate that γ-globin expression originated from SB100x integrated transgenes, an inverse PCR (iPCR) analysis was performed on genomic DNA from bone marrow mononuclear cells (MNCs) harvested at week 20 after transplantation. The iPCR protocol involves the digestion of genomic DNA with SacI, a re-ligation/circularization step, nested PCR and sequencing of vector/chromosome junctions (FIG. 24D). (FIG. 24E) shows three representative PCR products and the localization of the integration sites on chromosomes 4, 15, and X. Sequencing of the products demonstrated vector/chromosome junctions typical for SB100x mediated integration including the TA di-nucleotides at the vector IR/DR-chromosome junctions (FIG. 24F). In summary, in the ex vivo HSPC transduction study, the long globin LCR conferred high-level γ-globin expression originating from SB100x integrated transposons.

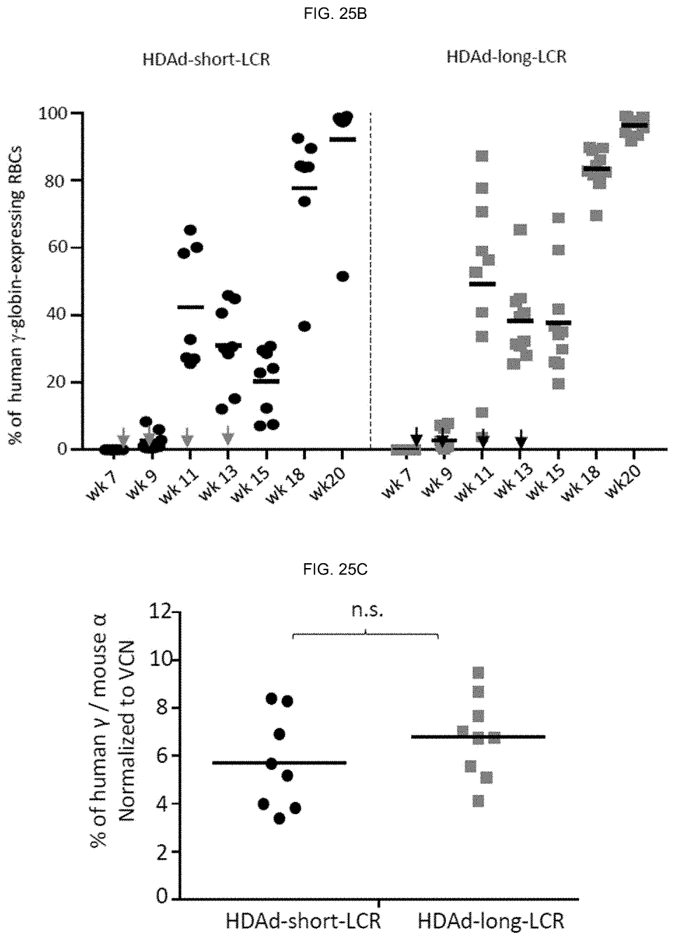

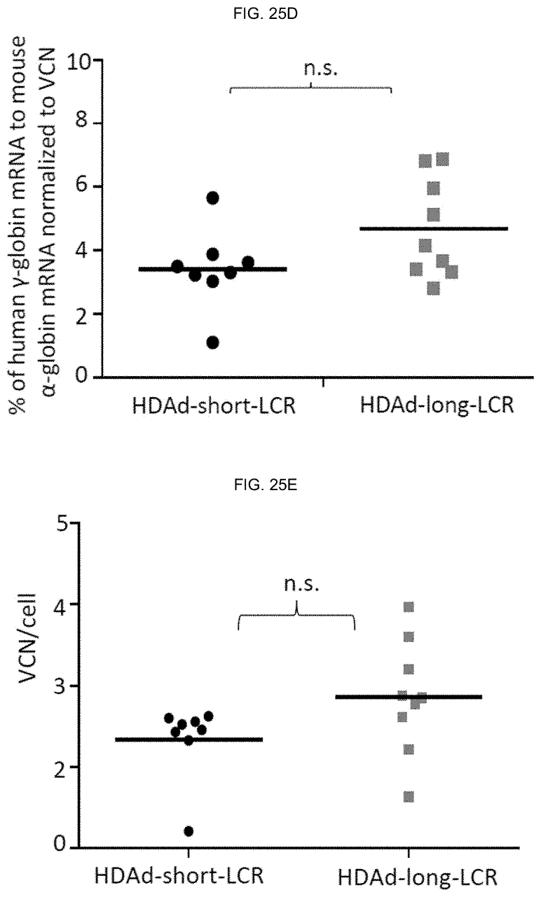

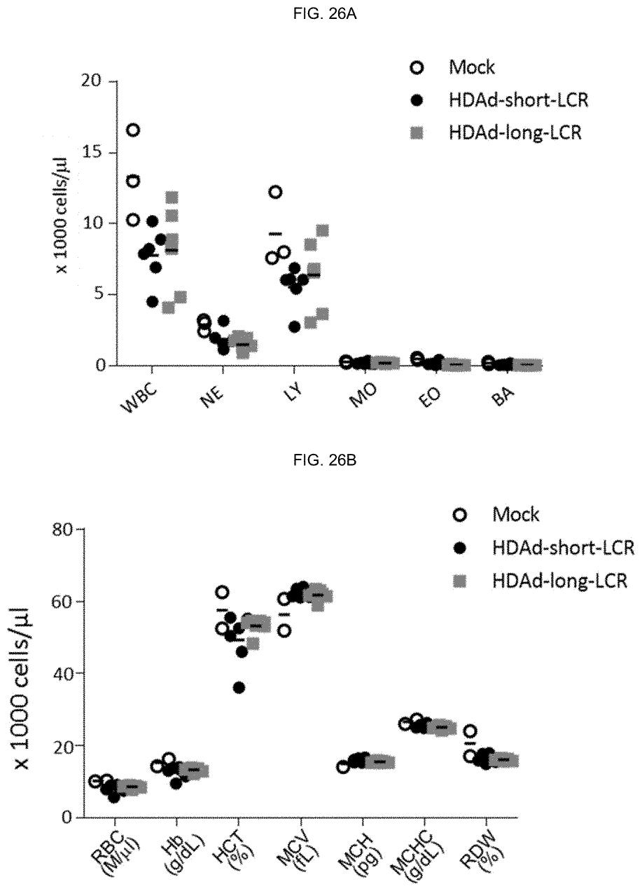

In vivo HSPC transduction in CD46b transgenic mice with HDAd5/35++ vectors containing the short vs long LCRs. A side-by-side comparison of HDAd-long-LCR and the previously used vector in Example 1 (Wang et al., J Clin Invest 129: 598-615, 2019; Li et al., Mol Ther Methods Clin Dev 9: 142-152, 2018) containing the miniLCR (herein referred to as “HDAd-short-LCR”) was performed (FIG. 23). CD46-transgenic mice were mobilized with G-CSF/AMD3100 and intravenously injected with the vectors, and five weeks later, subjected to in vivo selection (FIG. 25A). The percentage of γ-globin-positive red blood cells (RBCs) increased with each round of in vivo selection reaching >95% for both vectors at week 20 (FIG. 25B). HPLC performed on RBC lysates from week 20 samples did not show significant differences in percentages of γ-globin/adult mouse α-globin between the vectors (FIG. 25C). This was also reflected at the mRNA level (FIG. 25D).

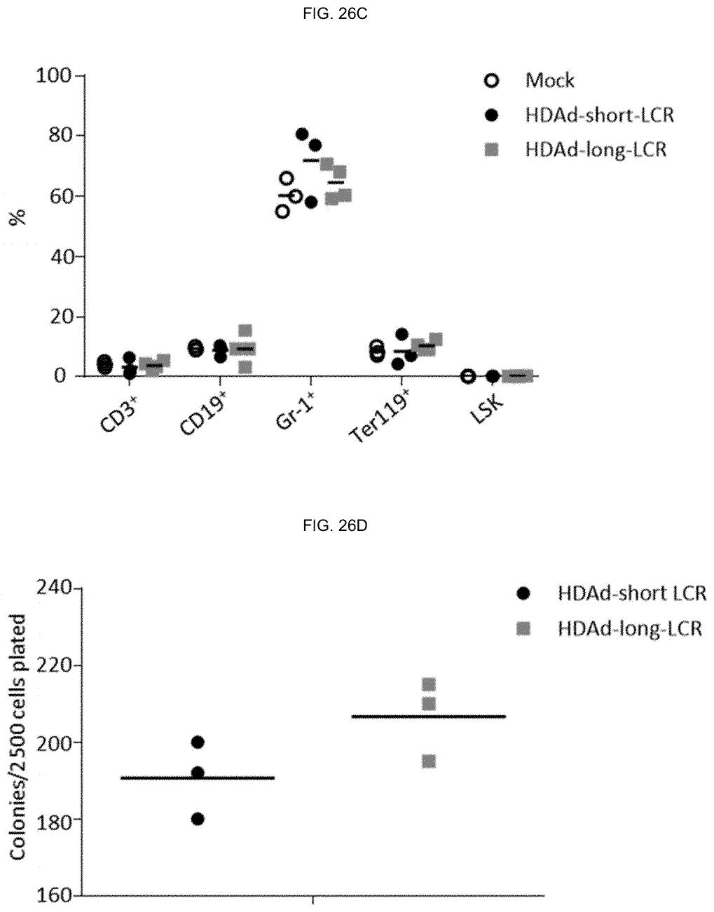

The vector copy number in bone marrow mononuclear cells (MNCs) measured at week 20 by qPCR, was 2.5 copies per cell (FIG. 25E) and not significantly different between the vectors. This indicated that the integration of the “long” 32.4 kb transposon was as efficient as the integration of the “short” 11.8 kb transposon. SB100x-mediated integration of the 32.4 kb transposon after in vivo HSPC transduction with the vectors did not cause hematological abnormalities (week 20) in spite of γ-globin expression in the vast majority of erythroid cells (FIG. 26B). The composition of cellular bone marrow (FIG. 26C) and the colony forming-potential of bone marrow Lin⁻ cells (FIG. 26D) were not significant between groups.

In a secondary transplant to demonstrate in vivo transduction and SB100x-mediated integration occurred in long-term repopulating HSPCs, the composition of cellular bone marrow (FIG. 26C) and the colony forming-potential of bone marrow Lin⁻ cells (FIG. 26D) were not significant between groups. Transplanted bone marrow Lin− cells were harvested at week 20 after in vivo HSPC transduction into lethally irradiated C57Bl/6 mice without hCD46 transgene). The ability of transplanted cells to drive the multi-lineage reconstruction in secondary recipients was assessed over a period of 16 weeks. As in the “primary” in vivo HSPC transduced mice, no effect of the high-level globin expression on the cellular composition of bone marrow or hematological parameters in the peripheral blood were observed.

Bone marrow Lin⁻ cells harvested at week 20 were also used to perform a genome-wide integration site analysis. In this assay, a linear amplification-mediated PCR (LAM-PCR) strategy is followed by sequencing of integration junctions (FIG. 27). The distribution of integration sites over the mouse genome is shown in FIG. 28A. The integrated transgene cassette was precisely processed, and the identified IR/DR chromosome junctions contained TA dinucleotides (FIG. 28B). The vast majority of integrations were within intergenic and intronic regions at a frequency of 83% and 17%, respectively (FIG. 28C). The integration was random without preferential integration in any given window of the whole mouse genome (FIG. 28D). No integration within or near a proto-oncogene was found. This SB100x-mediated integration pattern is in agreement with previous studies (Richter et al., Blood 128(18):2206-17, 2016; Neff et al, J Clin Invest 112(10):1581-8, 2003; Kemper et al., Clin Exp Immunol. 124(2):180-9, 2001; Zhang et al., PLoS One 8(10):e75344, 2013; Yant et al., Nat Biotechnol 20(10):999-1005, 2002).

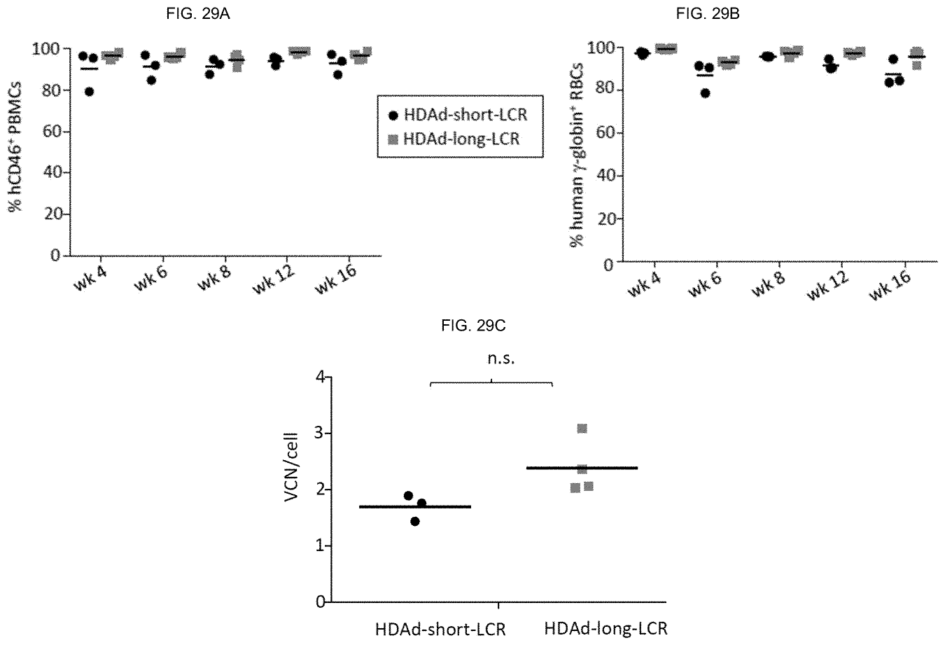

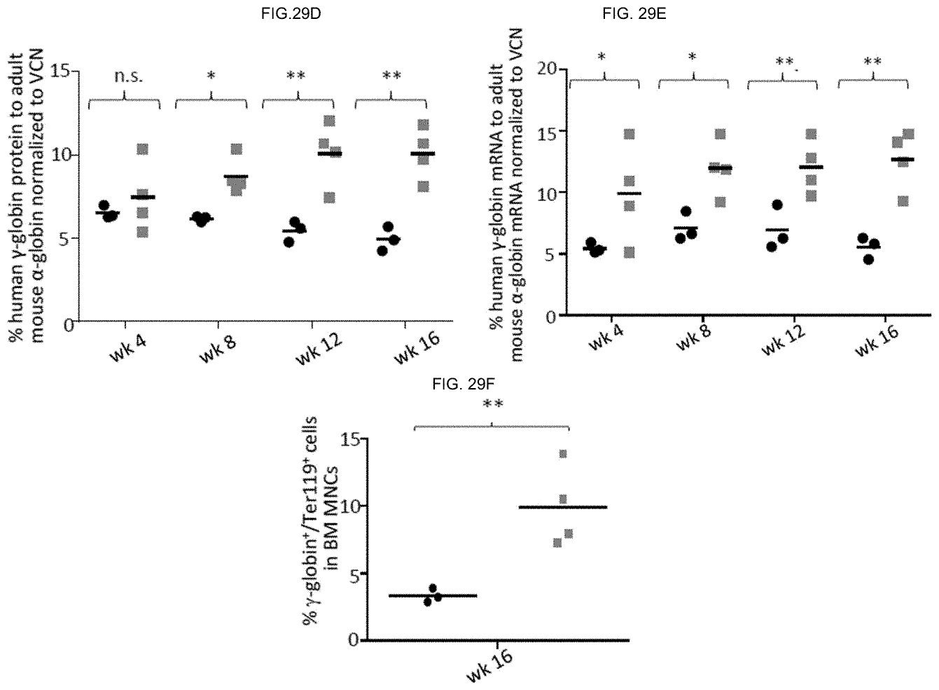

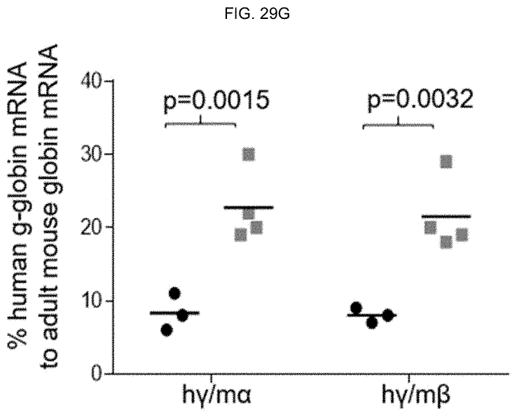

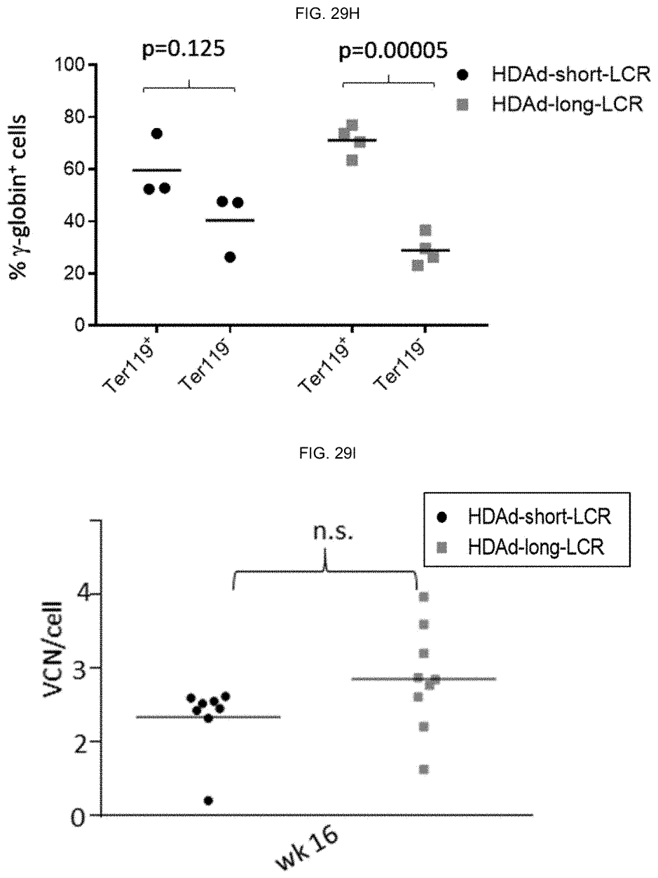

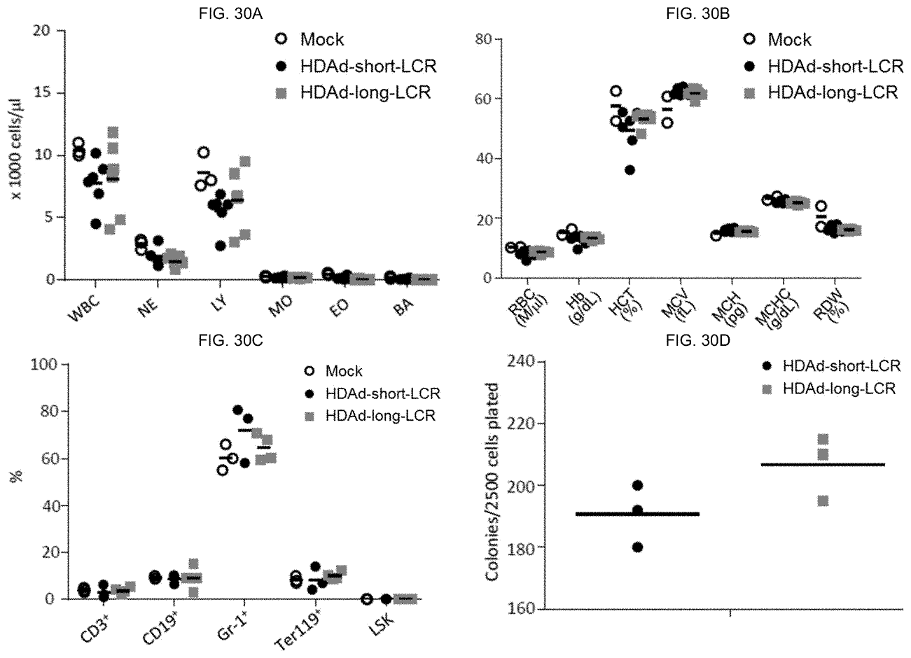

Analysis of secondary recipients. To demonstrate that in vivo transduction occurred in long-term repopulating HSPCs, bone marrow Lin⁻ cells harvested at week 20 after in vivo HSPC transduction were transplanted with HDAd-short-LCR and HDAd-long-LCR, into lethally irradiated C57Bl/6 mice (without the hCD46 transgene). The ability of transplanted cells to drive the multi-lineage reconstitution in secondary recipients was assessed over a period of 16 weeks. Engraftment rates based on CD46 expression in PBMCs were 95% and remained stable (FIG. 29A). γ-globin marking of RBCs measured by flow cytometry was in the range of 90 to 95% and stable (FIG. 29B). There was no significant difference between the two vectors in the percentage of γ-globin⁺ RBCs. The average integrated vector copy number also did not differ significantly between the two vectors indicating that integration of both transposons in long-term repopulating cells was equally efficient (FIG. 29C). Interestingly, the percentage of γ-globin to mouse adult globin chains increased over time for the HDAd-long-LCR vector reaching 20-25% of mouse α-globin (FIGS. 29D and 29E). In contrast, the percentage of γ-globin/mouse α-globin in secondary recipients of HDAd-short-LCR transduced bone marrow cells did not increase. The percentage of γ-globin expressing erythroid cells was significantly higher for HDAd-long-LCR (FIG. 29F). In addition to conferring higher γ-globin expression levels, the long LCR also provided more stringent erythroid-specific expression as shown by a significantly higher percentage of γ-globin expressing bone marrow cells in the erythroid (Ter119⁺) fraction vs the non-erythroid fraction (Ter119⁻) (FIG. 27H). The vector number copy per cell in bone marrow MNCs were not statistically significant between HDAd-short-LCR and HDad-long-LCR when harvested at week 16 after in vivo HSPC transduction (FIG. 27I). As in the “primary” in vivo HSPC transduced mice, no effect of high-level globin expression on the cellular composition of the bone marrow or hematological parameters in the peripheral blood were observed (FIGS. 30A-30D).

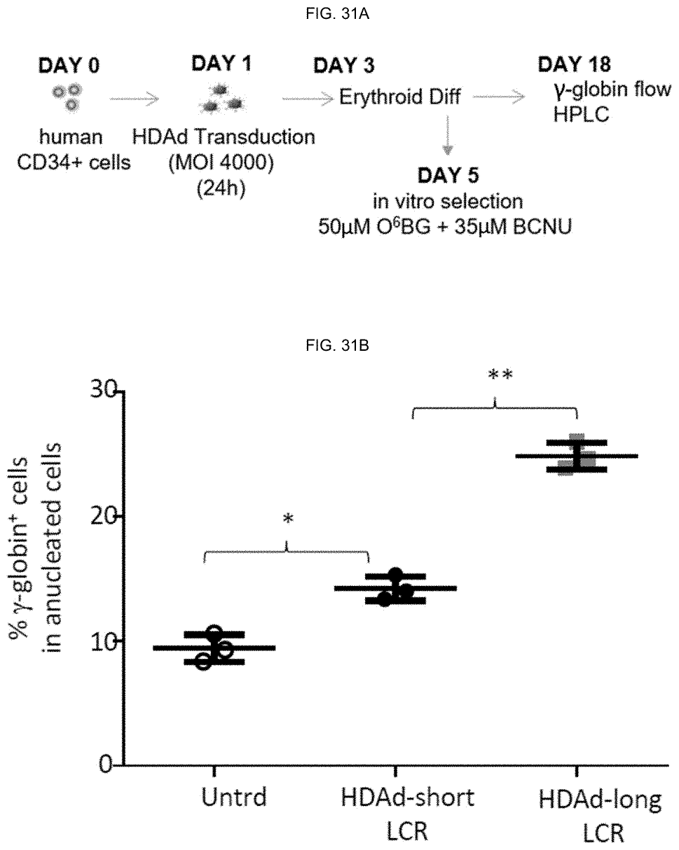

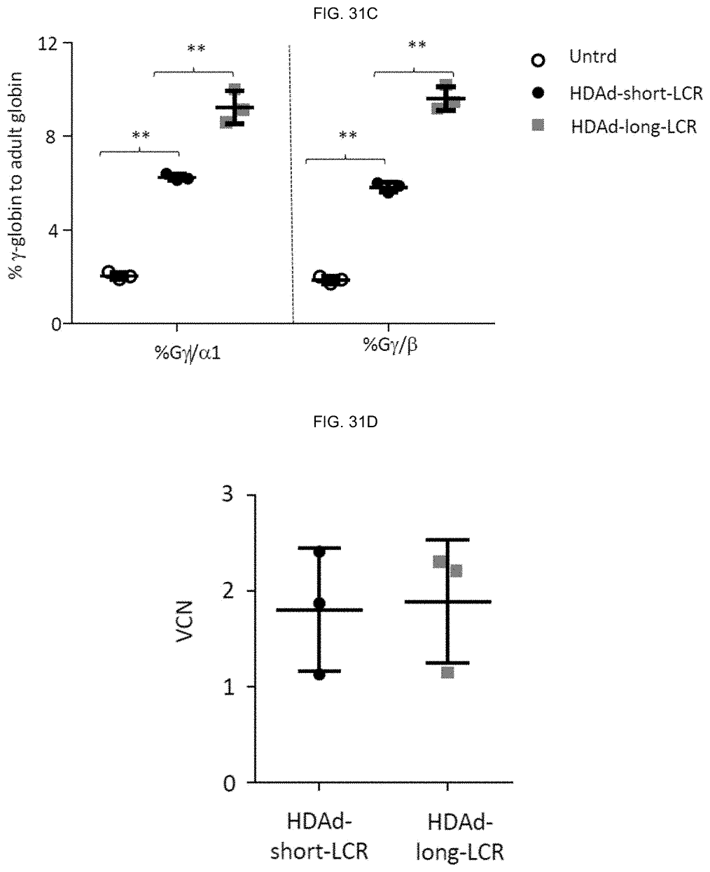

Comparison of the two vectors after human CD34+ transduction, in vitro selection, and erythroid differentiation. The function of the human β-globin LCR in a heterologous system like mouse erythroid cells could be suboptimal due to lack of conservation of transcription factors that bind within the LCR. An in vitro study in human cells was, therefore, performed (FIG. 31A). Human CD34+ cells obtained from GCSF-mobilized healthy donors were transduced with HDAd-long-LCR+HDAd-SB or HDAd-short-LCR+HDAd-SB at a total MOI of 4000 vp/cells, i.e. a MOI that confers the transduction of the majority of CD34+ cells (Li et al., Mol Ther Methods Clin Dev 9: 390-401, 2018). Transduced cells were then subjected to erythroid differentiation (ED) and O⁶BG/BCNU selection for cells with integrated transgenes. During expansion of transduced cells over 18 days, most of episomal vectors are lost. At the end of ED, significantly higher percentages of γ-globin⁺ anucleated cells (i.e. reticulocytes that lost the nucleus) were found for the HDAd-long-LCR+HDAd-SB setting by flow cytometry (FIG. 31B). HPLC analysis also demonstrated significantly higher γ-globin chain levels in HDAd-long-LCR+HDAd-SB-transduced cells (FIG. 31C).

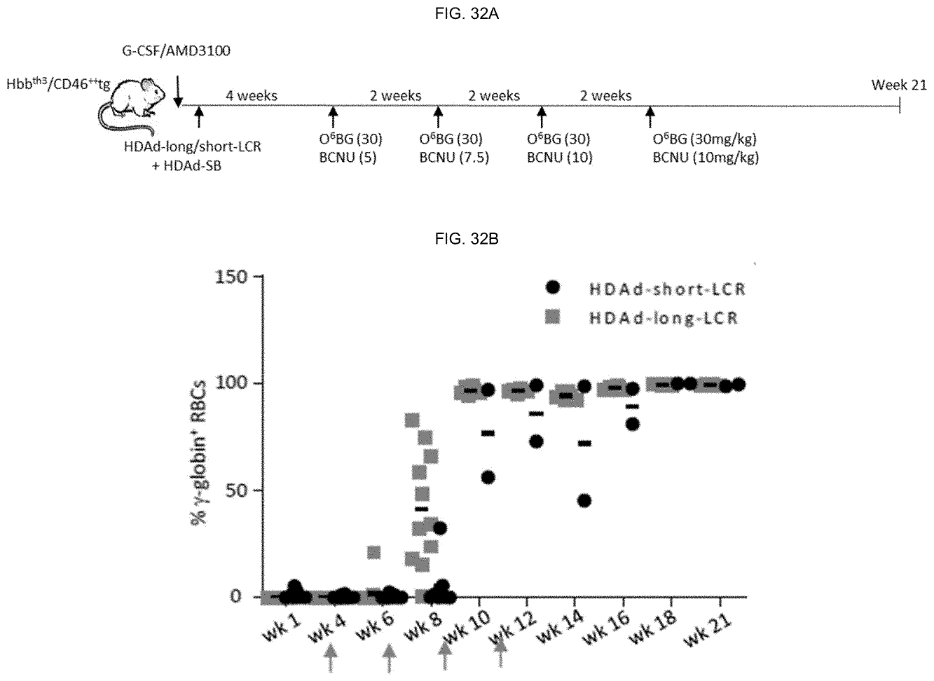

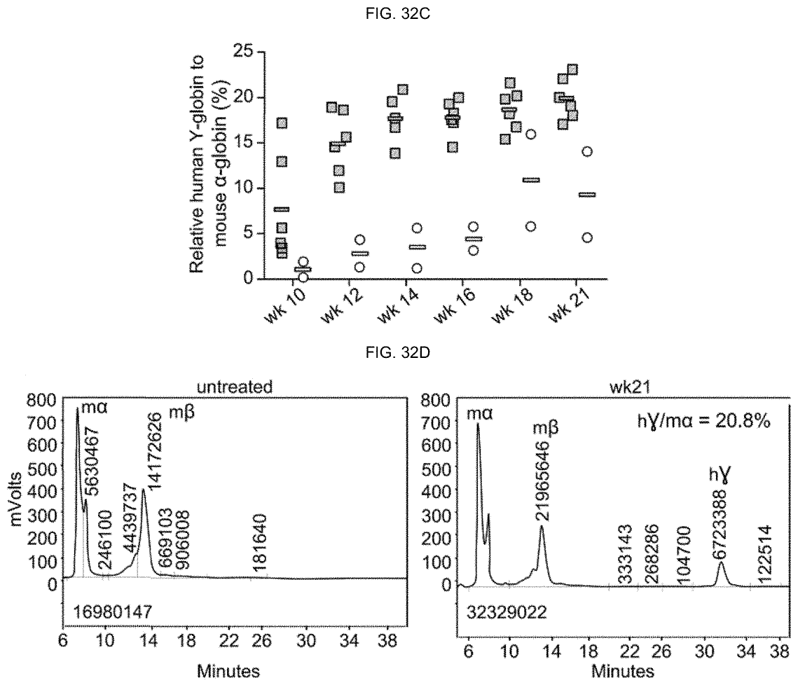

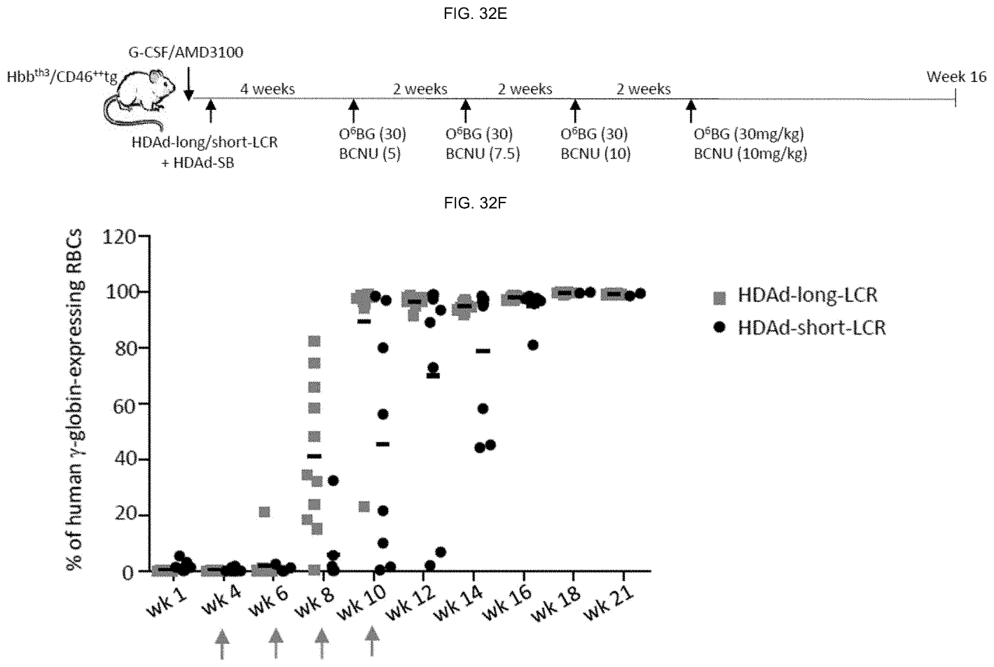

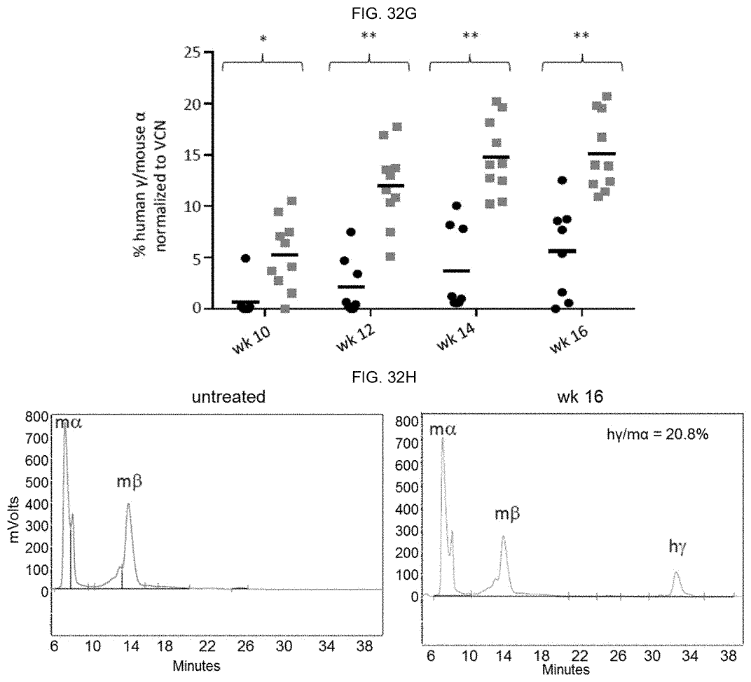

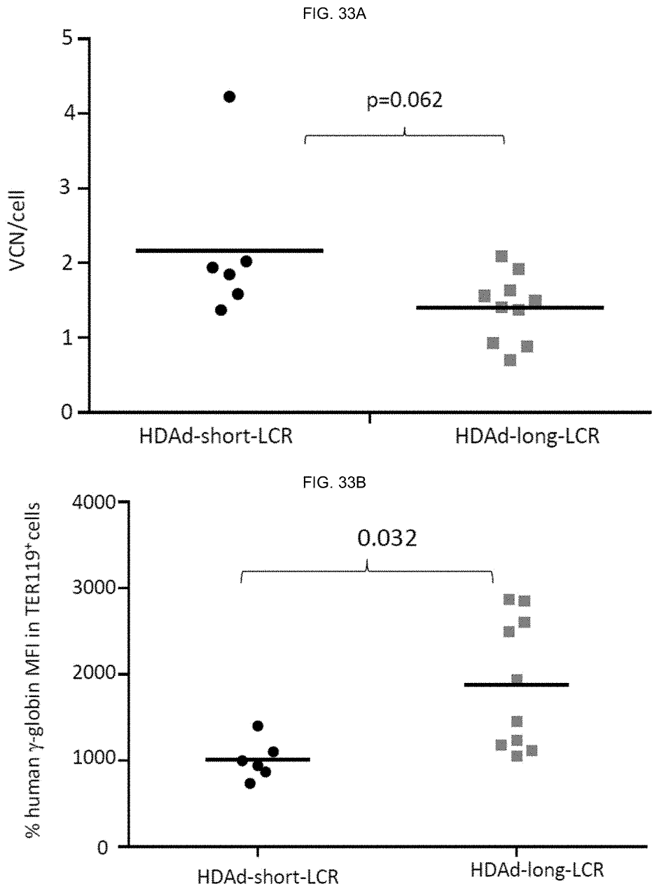

HDAd-short-LCR vs HDAd-long-LCR in vivo HSPC transduction studies in a mouse model of thalassemia intermedia [symbol] -globin levels. For these studies (over 4 rounds) (CD46+/+) mice were bred with Hbbth³ mice heterozygous for the mouse Hbb-beta1 and -beta2 gene deletion (Yoshida et al., Sci Rep 7:43613, 2017). Resulting Hbb^{th3}/CD46+/+ mice has the typical phenotype of thalassemia intermedia (Wang et al., J Clin Invest, 129: 598-615. 2019). Hbb^{th3}/CD46+/+ mice were mobilized, intravenously injected with HDAd-long-LCR and HDAd-short-LCR vector systems, and four weeks later subjected to in vivo selection (FIGS. 32A and 32E). Importantly, [symbol] -globin marking in peripheral red blood cells was on average 40% already after the second cycle of in vivo selection, reached >90% in 9 out of 10 mice after the third cycle, and plateaued near 100% in all mice at week 12 after in vivo transduction with HDAd-long-LCR (FIGS. 32B and 32F). in contrast, for mice transduced with HDAd-short-LCR, it required four in vivo selection cycles to reach 100% [symbol] -globin marking in RBCs in 2 out of 7 mice and 100% marking was achieved only at week 16 post-transduction. At 100% marking rate, the percentage of human [symbol] -globin vs adult mouse α-globin chains (measured by HPLC) increased over time for both vectors (most likely due to the disease background) reaching an average of 22% (max: 35%) and 11% (max: 19%) by week 16 after in vivo transduction with HDAd-long-LCR and HDAd-short-LCR, respectively (FIGS. 32G and 32H; FIGS. 32C and 32D for week 21 data). Similar to what was observed in CD46tg mice, analysis of bone marrow mononuclear cells showed comparable VCNs for both vectors and higher globin expression levels in erythroid cells for HDAd-long-LCR (FIG. 33). In summary, these data demonstrate the superiority of HDAd-long-LCR over HDAd-short-LCR by i) requiring less intense in vivo selection to reach 100% marking and achieving γ-globin levels in RBCs, that, in theory, should be curative in patients with SCD and thalassemia major.

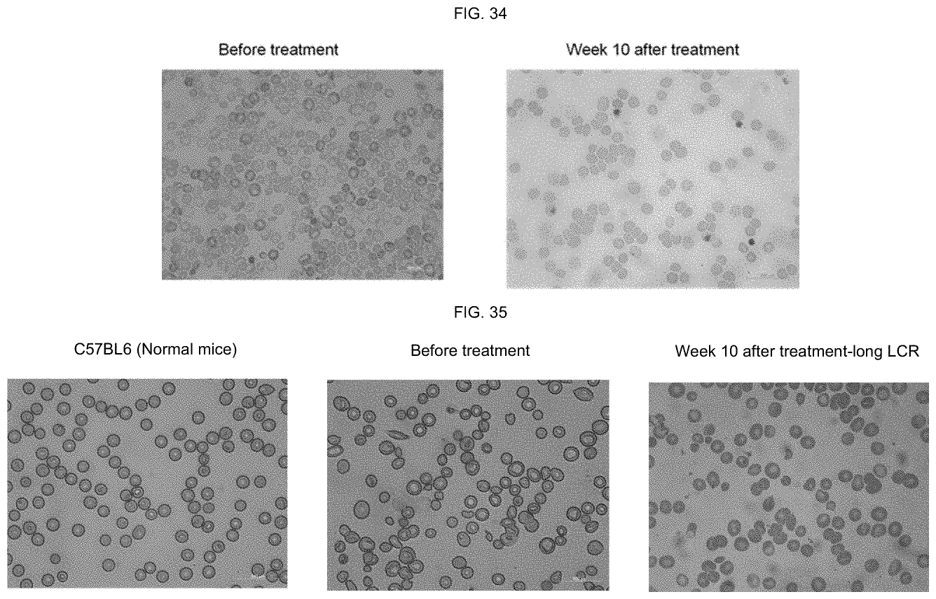

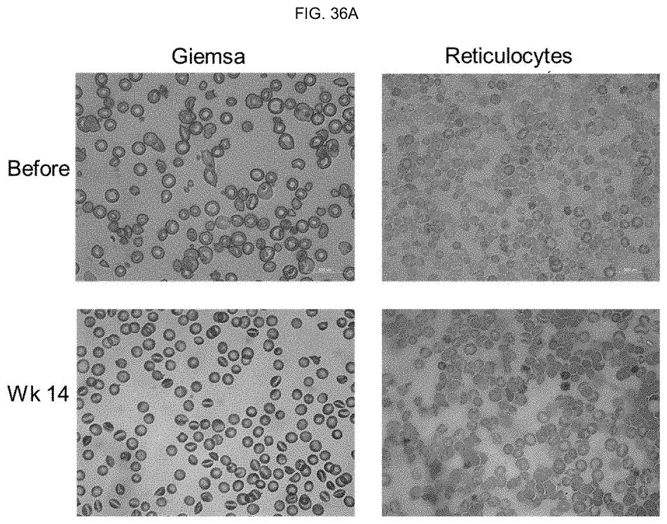

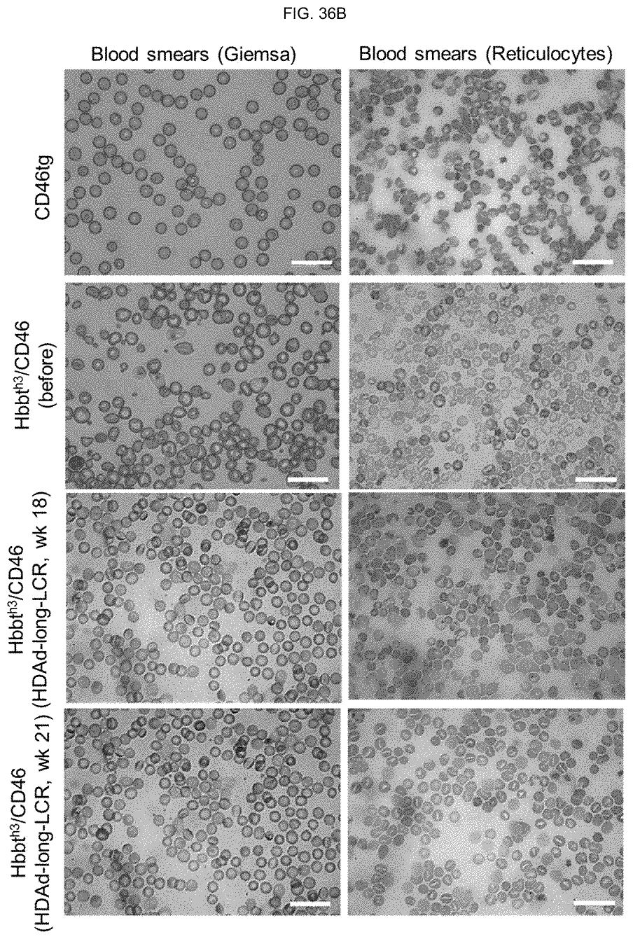

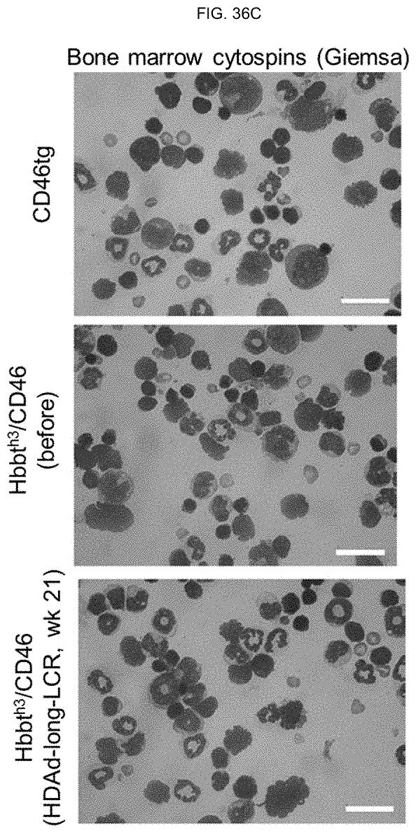

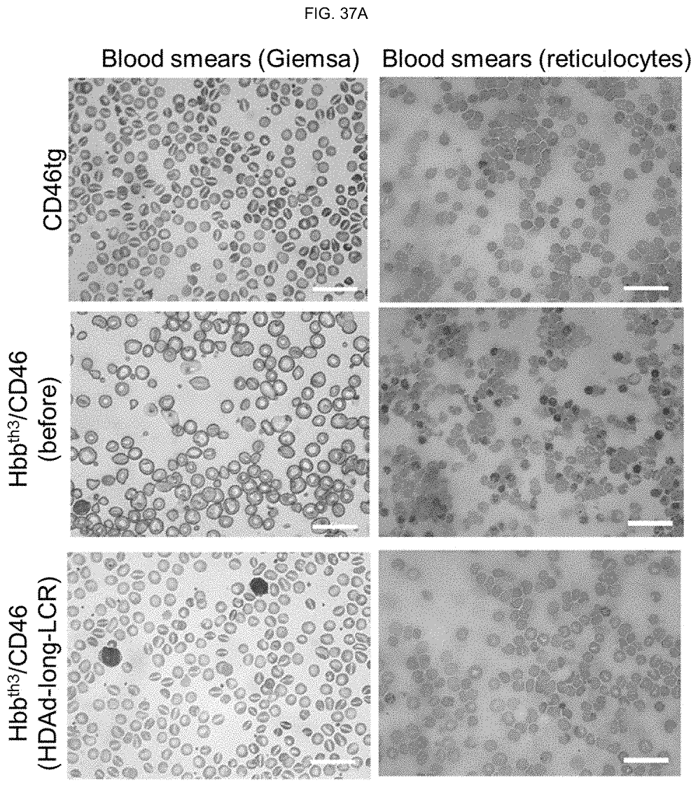

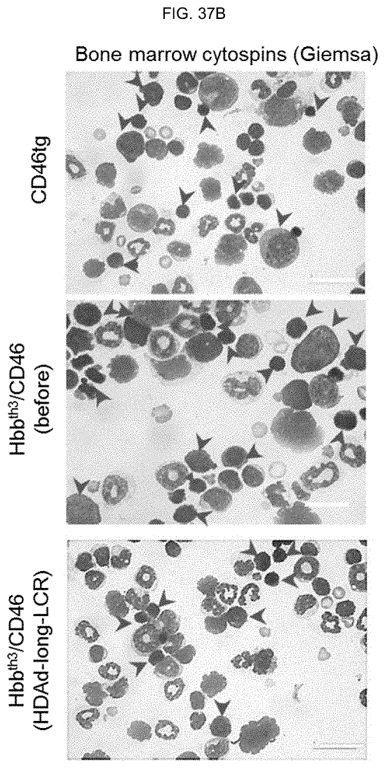

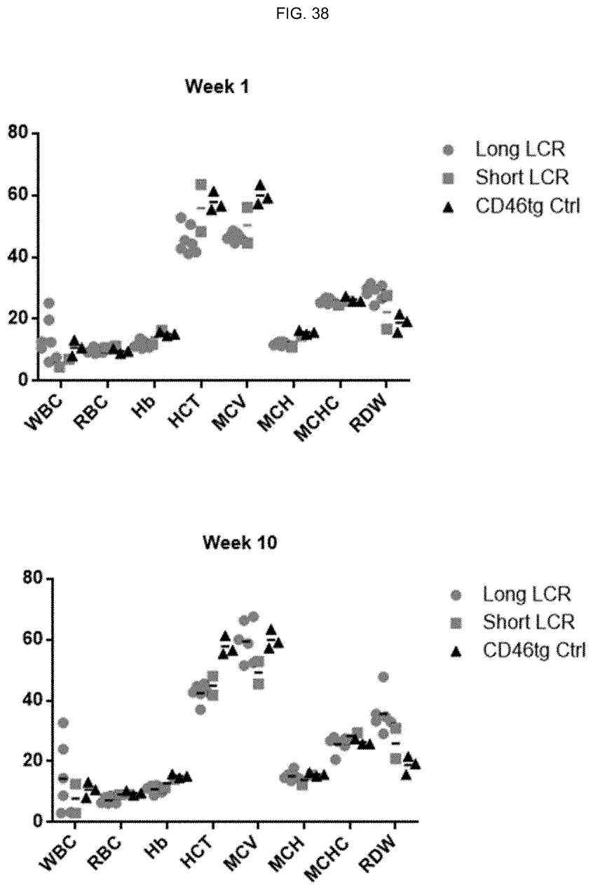

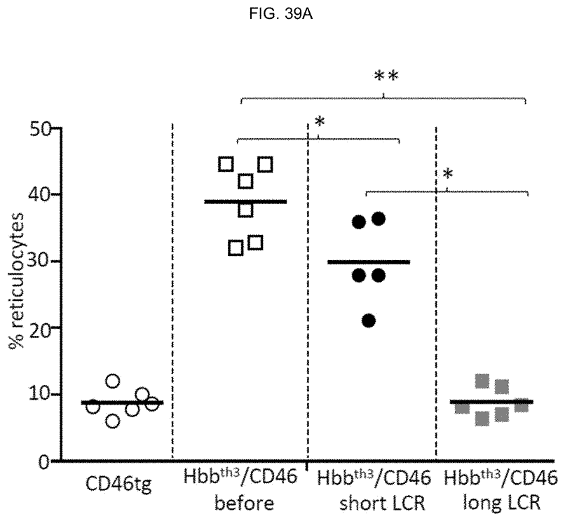

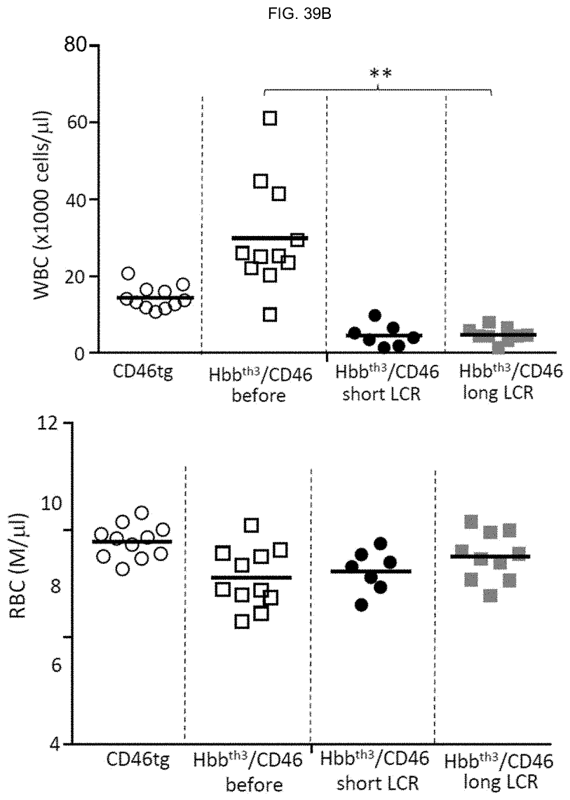

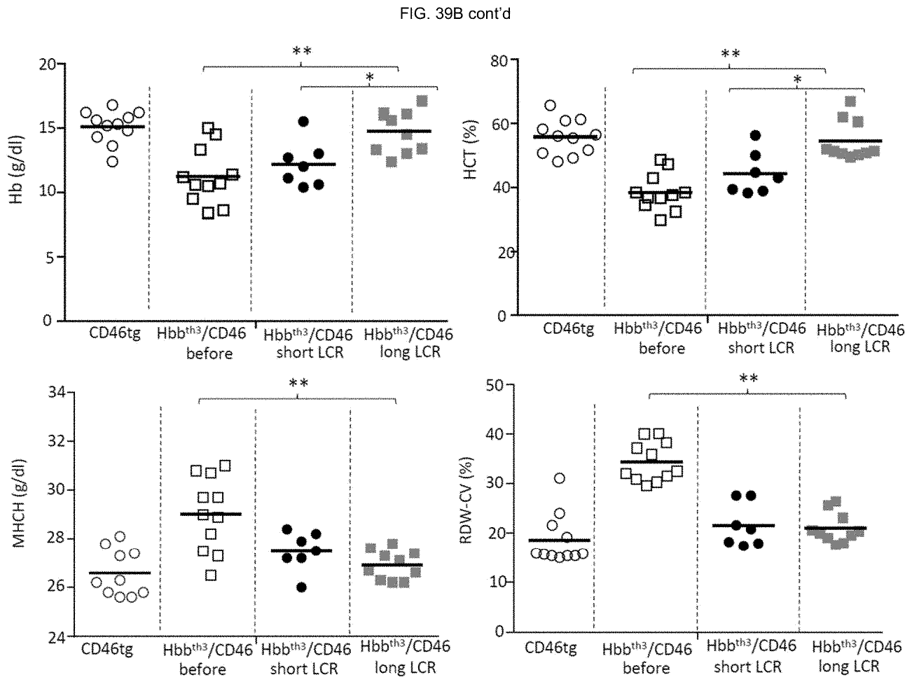

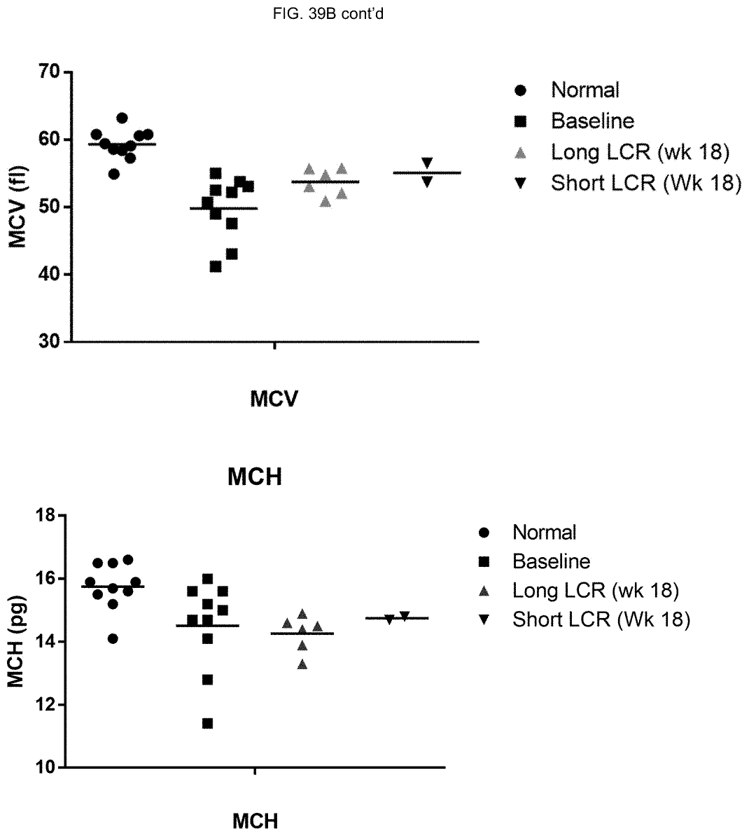

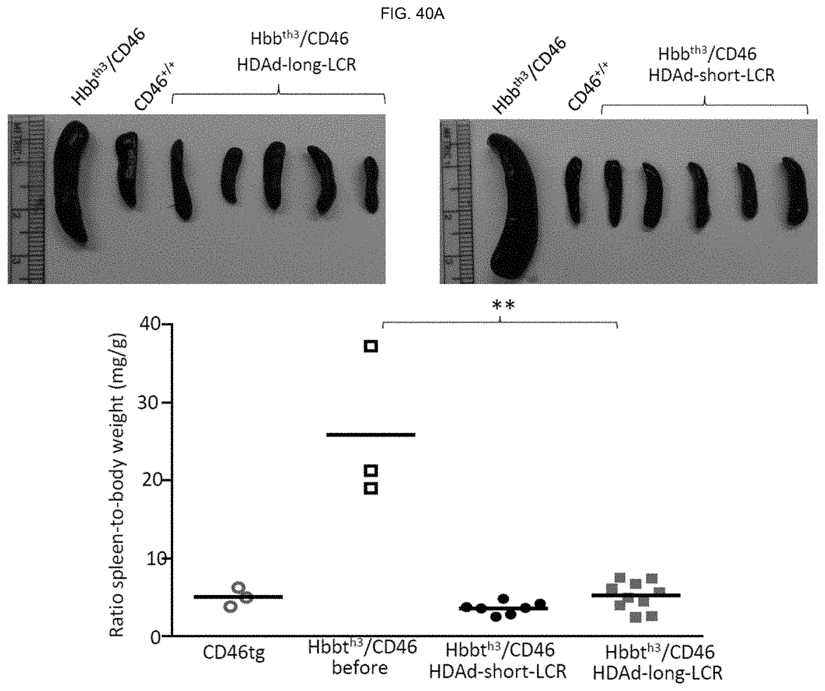

Correction of hematological parameters. Phenotypic correction is shown at different time points. Micrographs comparing the normalized erythrocyte morphology of C57BL6 and Townes SCA mice, before treatment and at week 10 after treatment with long LCR (FIG. 34) and micrographs showing the normalized erythropoiesis (reticulocyte count) for Townes mice, before treatment, and Townes mice at week 10, after treatment with long LCR are shown (FIG. 35). At week 14, blood cell morphology stained with Giemsa stain and May-Grunwald stain are shown (FIG. 36A). At week 16 after treatment, mice were sacrificed. Indicative of the reversal of the thalassemic phenotype in peripheral blood smears of the treated Hbb^{th3}/CD46+/+ mice, the hypochromic, highly fragmented and anisopoikilocytic baseline RBCs were replaced by near normochromic, well-shaped RBCs (FIG. 37A, left panels, see FIG. 36B for week 21 data). The level of reticulocytes in peripheral blood was comparable to normal CD46tg mice (FIG. 37A, right panels, see also FIG. 39). Analogous data for week 21 can be found in FIG. 36B, in the right panel. In bone marrow cytospins, in contrast to the blockade of erythroid lineage maturation in bone marrow of Hbb^{th3}/CD46+/+ mice, represented by the prevalence of basophilic erythroblasts, in cytospins from control and treated Hbb^{th3}/CD46+/+ mice, maturing polychromatic and orthochromatic erythroblasts predominated (FIG. 37B, see FIG. 36C for week 21 data). The normalized erythrocyte parameters of mice transduced with long LCR, short LCR, and control CD46tg vectors are shown (FIG. 38). Hematological parameters at week 16 post in vivo transduction were significantly improved compared to pre-treatment parameters for both vectors (FIGS. 38, 39A). For white blood cells, red blood cells, MCHC, MCV, and RDW-CV they were indistinguishable from the CD46tg controls (FIG. 39A). However, there were significant differences in favor of animals treated with HDAd-long-LCR vector vs HDAd-short-LCR, specifically, the percentage of reticulocytes in peripheral blood was 40.9 vs 26.8 vs 9.2% for non-treated, HDAd-short-LCR, and HDAd-long-LCR-treated Hbb^{th3}/CD46+/+ mice, respectively (FIG. 38). Furthermore, hemoglobin levels and hematocrit were higher for the HDAd-long-LCR-treated group.

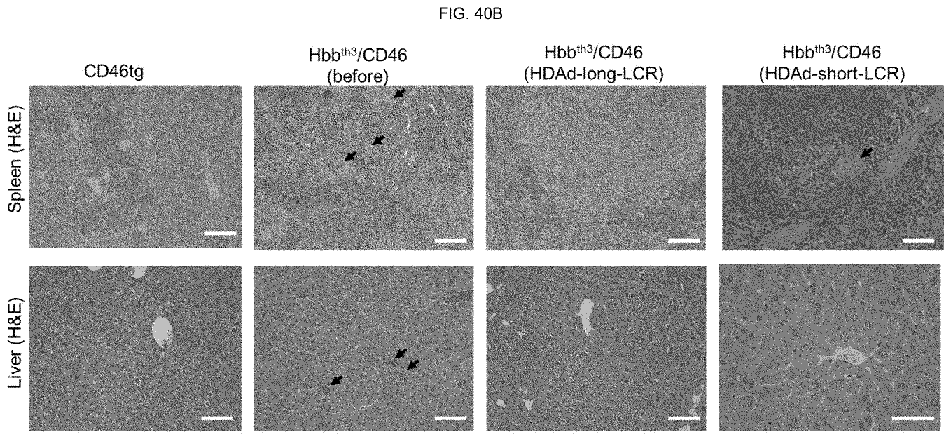

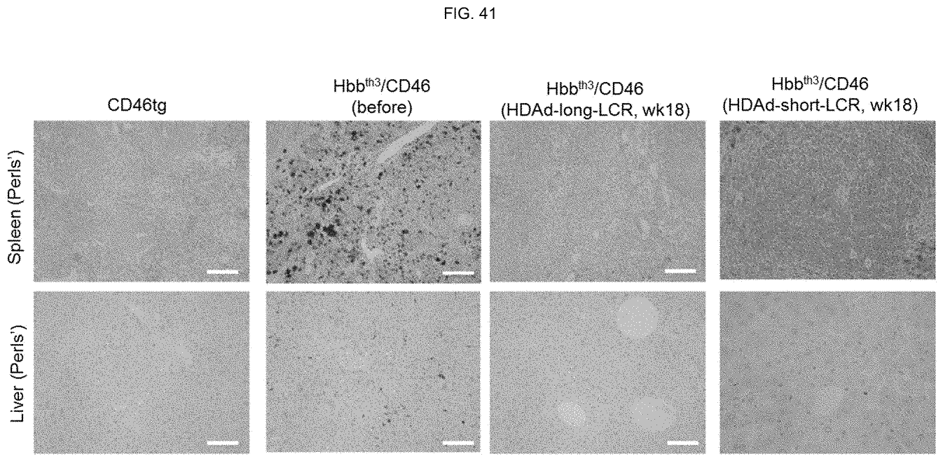

Correction of extramedullary hematopoiesis and hemosiderosis. Spleen size, a measurable characteristic of compensatory hemopoiesis was reduced to normal in animals treated with both vectors, whereby there was no significant difference between HDAd-long-LCR and HDAd-short-LCR (FIG. 40A). In contrast to Hbb^{th3}/CD46+/+ mice, no foci of extramedullary erythropoiesis were observed on spleen and liver sections after treatment with HDAd-long-LCR and only limited extramedullary erythropoiesis was detected in the HDAd-short-LCR-treated mice (FIG. 40B). Intense hemosiderosis in spleen and liver was prominent in the untreated Hbb^{th3}/CD46+/+ mice (FIG. 41, second panel. Signals after Perl's staining of the tissues were comparably low for CD46tg (FIG. 41, first panel) and HDAd-long-LCR treated Hbb^{th3}/CD46+/+ mice (FIG. 41, third panel(, whereas 2.7(+/−0.8)-fold more blue spots per cm² spleen tissue were counted for HDAd-short-LCR vs HDAd-long-LCR-treated animals (N=5).

In summary, reticulocytes, blood parameters, extracellular hematopoiesis and hemosiderosis in HDAd-long-LCR-treated animals were not significantly different from control CD46tg mice, indicating a complete phenotypic correction. Furthermore, HDAd-long-LCR proved to be superior over HDAd-short-LCR in curing thalassemic mice in several phenotypic parameters, most likely due to higher γ-globin levels expressed from the long-LCR.

Comparison of the two vectors after human CD34+transduction and erythroid differentiation. To consolidate the data in mice, an in vitro study was performed in human cells (FIG. 31A). Human CD34+ cells obtained from GCSF-mobilized healthy donors were transduced with HDAd-long-LCR+HDAd-SB or HDAd-short-LCR+HDAd-SB at a total MOI of 4000 vp/cells, i.e. a MOI that confers the transduction of the majority of CD34+ cells (Yang et al., Proc Natl Acad Sci USA. 92(25):11608-12, 1995). Transduced cells were then subjected to erythroid differentiation (ED) and O⁶BG/BCNU selection for cells with integrated transgenes. During expansion of transduced cells over 18 days, most of episomal vectors are lost.

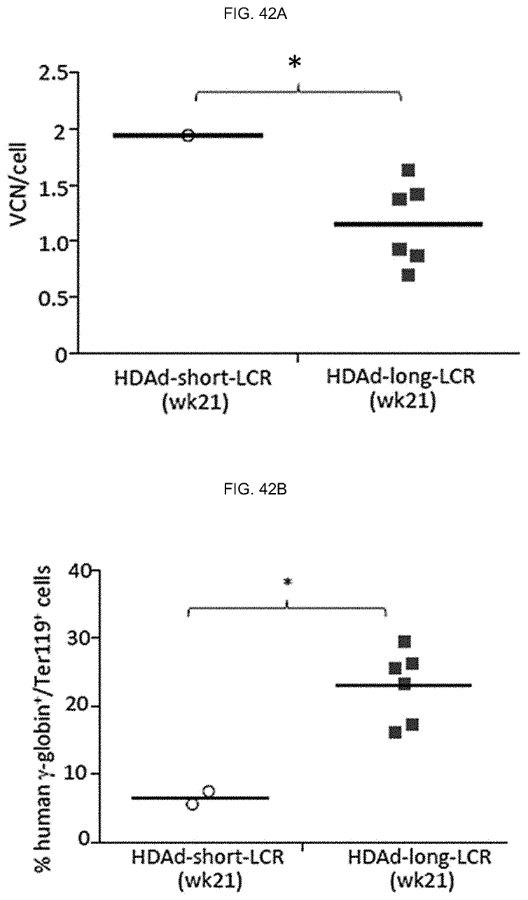

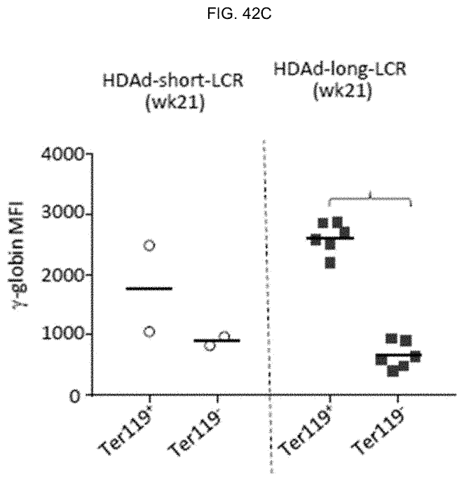

Bone marrow was harvested at week 21 after in vivo HSC transduction of Hbb^{th3}/CD46tg mice. (FIG. 42A) Vector copy number per cell in bone marrow MNCs. The difference between the two groups is not significant but could become significant if analyzed with greater sample size. (FIGS. 42B, 42C) Erythroid specificity of γ-globin expression. (FIG. 42B) Percentage of γ-globin expressing erythroid (Ter119⁺) and non-erythroid (Ter119⁻) cells. *p<0.05. Statistical analyses were performed using two-way ANOVA.

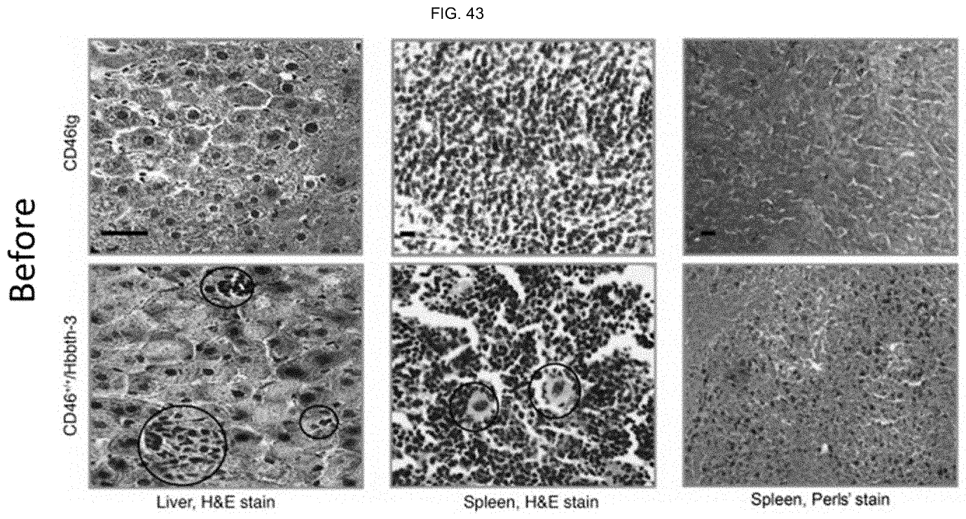

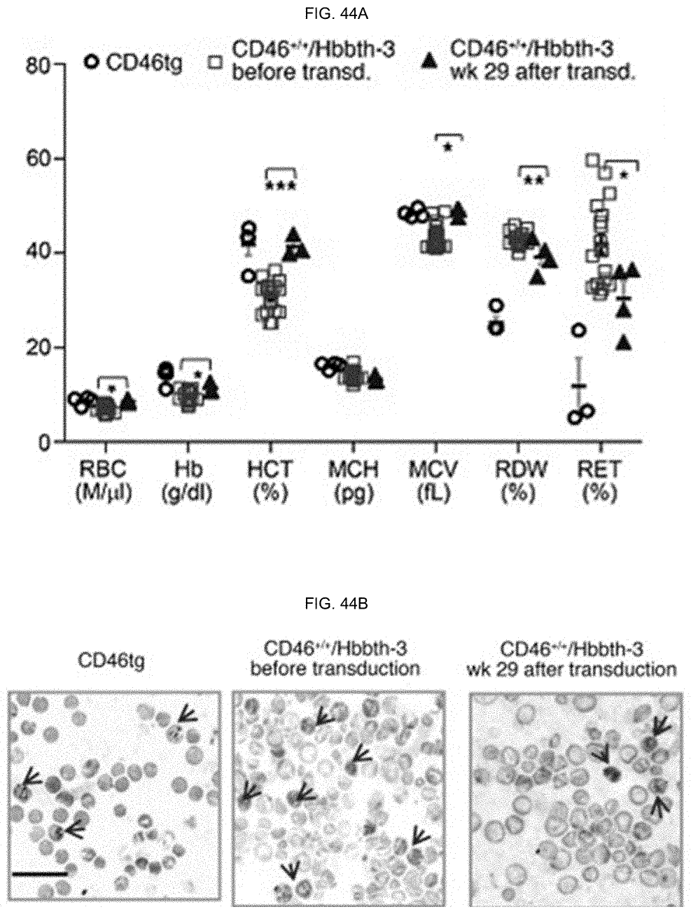

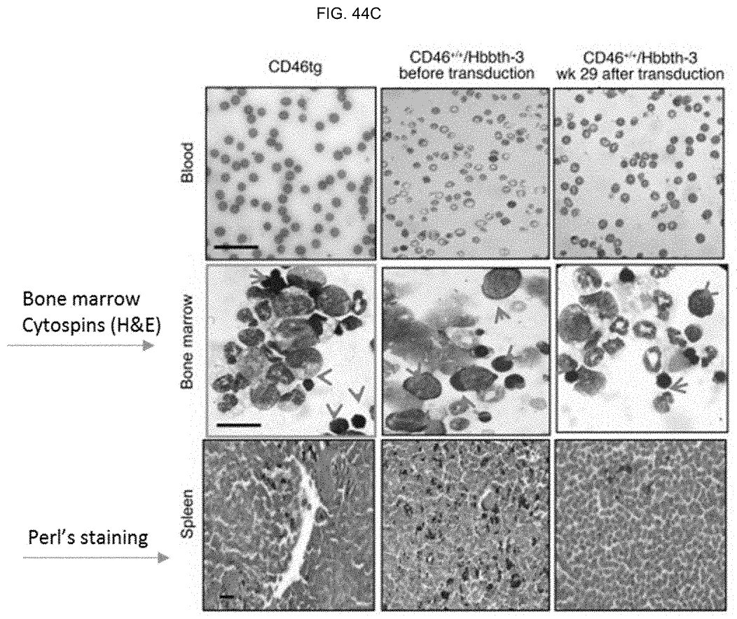

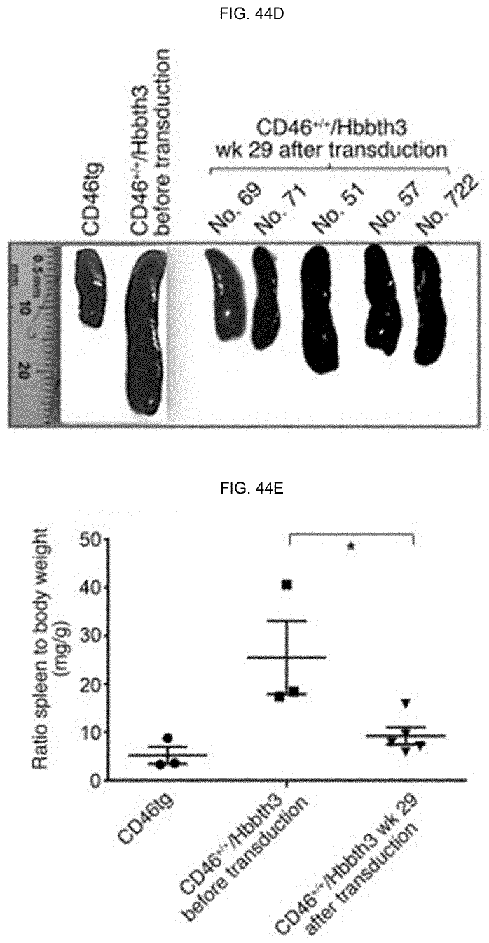

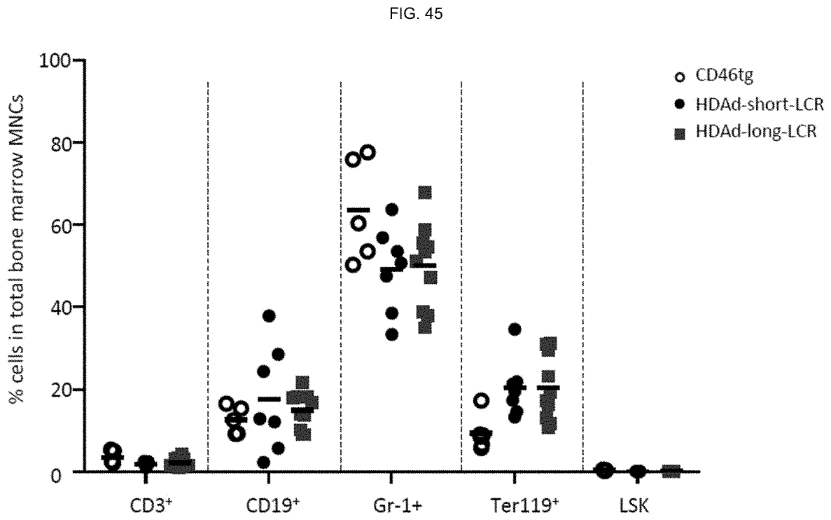

Extramedullary hemopoiesis by hematoxylin/eosin staining in liver and spleen sections from CD46tg and CD46^{+/+}/Hbb^{th-3} mice prior to administration of an adenoviral donor vector (FIG. 43). Iron deposition is shown by Perl's staining as cytoplasmic blue pigments of hemosiderin in spleen.

At the end of ED, significantly higher percentages of γ-globin⁺ enucleated cells (i.e. reticulocytes that lost the nucleus) were found by flow cytometry (FIG. 31B) and also significantly higher [symbol] -globin chain levels by HPLC in the HDAd-long-LCR vs HDAd-short-LCR setting (FIG. 31C). The vector copy number measured at day 18 was 2 for both vectors (FIG. 31D).

In summary, the ex vivo and in vivo HSPC transduction studies with mice as well as the in vitro studies with human HSPCs support the relevance of HDAd-long-LCR for gene therapy of hemoglobinopathies.

Discussion This Example describes work relevant to the clinical development of an in vivo HSPC gene therapy approach that does not require leukapheresis, myeloablation and HSPC transplantation (Richter et al, Blood. 128(18):2206-17, 2016). These are critical obstacles to a wide-spread application for ex vivo HSPC gene therapy of hemoglobinopathies, particularly in older patients and patients with comorbidities. The safety and efficacy of this approach has been demonstrated in several murine disease models (Wang et al., J Clin Invest. 129(2):598-615, 2019; Wang et al., Blood Adv. 3(19):2883-94, 2019; Li et al., Mol Ther Methods Clin Dev. 9:390-401, 2018) and, recently, in non-human primates (Li et al., 23rd Annual ASGCT meeting. 2020; abstract #546). In both species, a major problem associated with intravenous HDAd5/35++injection has been addressed, namely acute innate immune responses, by a prophylaxis regimen that blocked pro-inflammatory cytokines.

Reaching curative γ- or β-globin expression levels in thalassemia major and SCD patients in ex vivo HSPC gene therapy settings is still a challenge. It requires approaches to increase the number of integrated transgene copies by either optimizing the HSPC transduction process or by increasing the multiplicity of infection. Increasing the VCN however, bears the risk of inducing genotoxicity. Other attempts focus on further optimizing globin expression cassettes (Li et al., Cancer Res. 80(3):549-60, 2020). With high-payload capacity HDAd vectors, there is an opportunity to go beyond the genome size limitations set for lenti- and rAAV vectors. The present study demonstrates that curative levels of γ-globin can be achieved in RBCs by in vivo HSPC gene therapy with an integrating HDAd5/35++vector accommodating β-globin LCR/promoter elements with a total length of 29 kb.

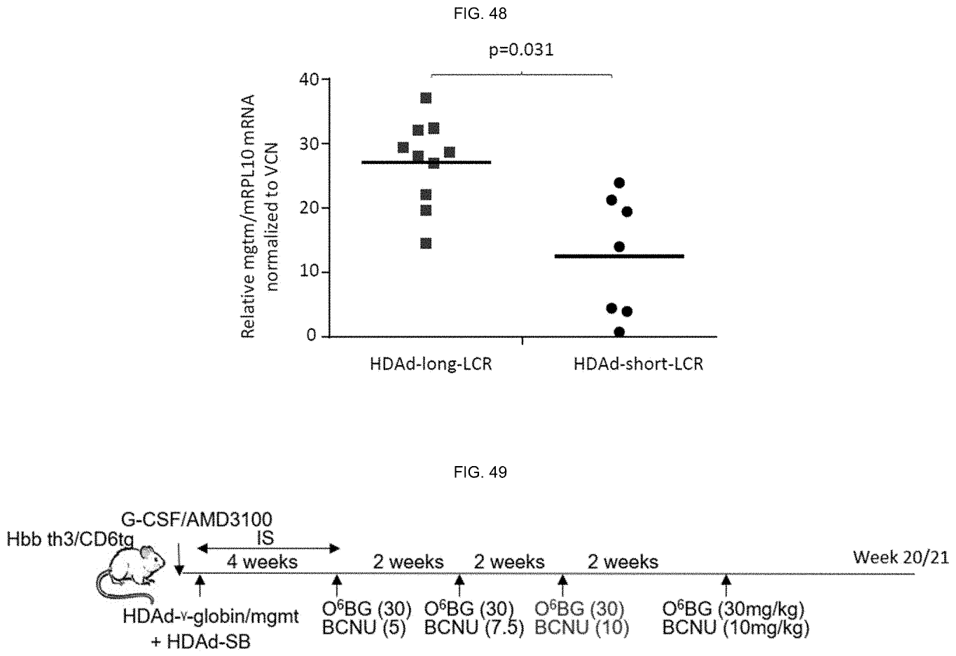

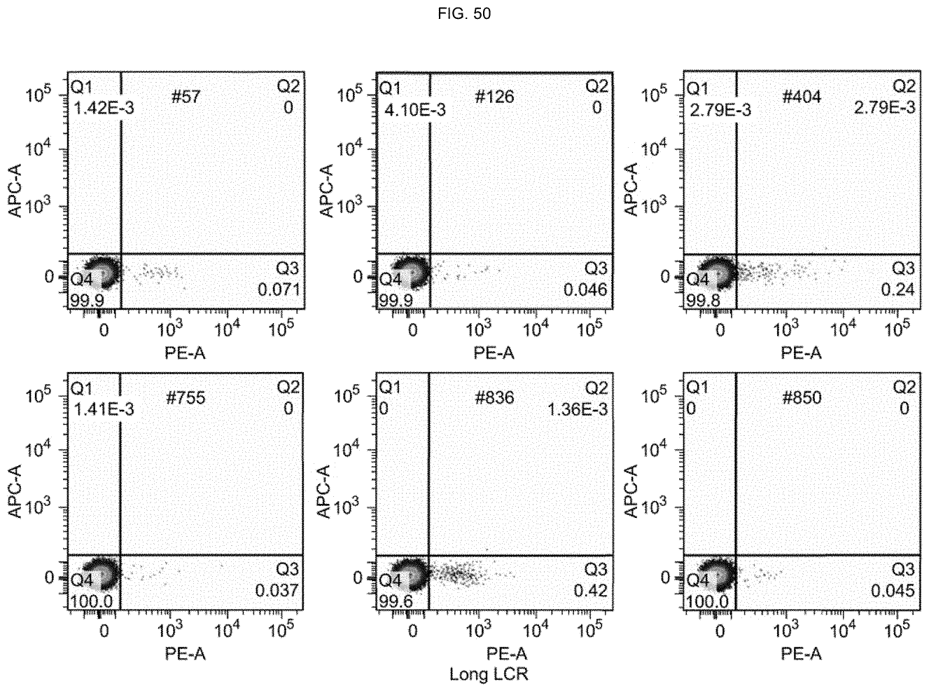

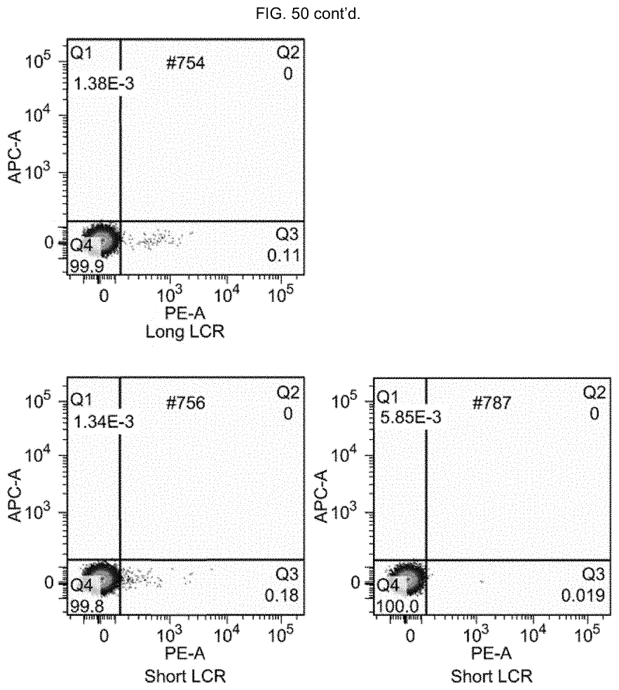

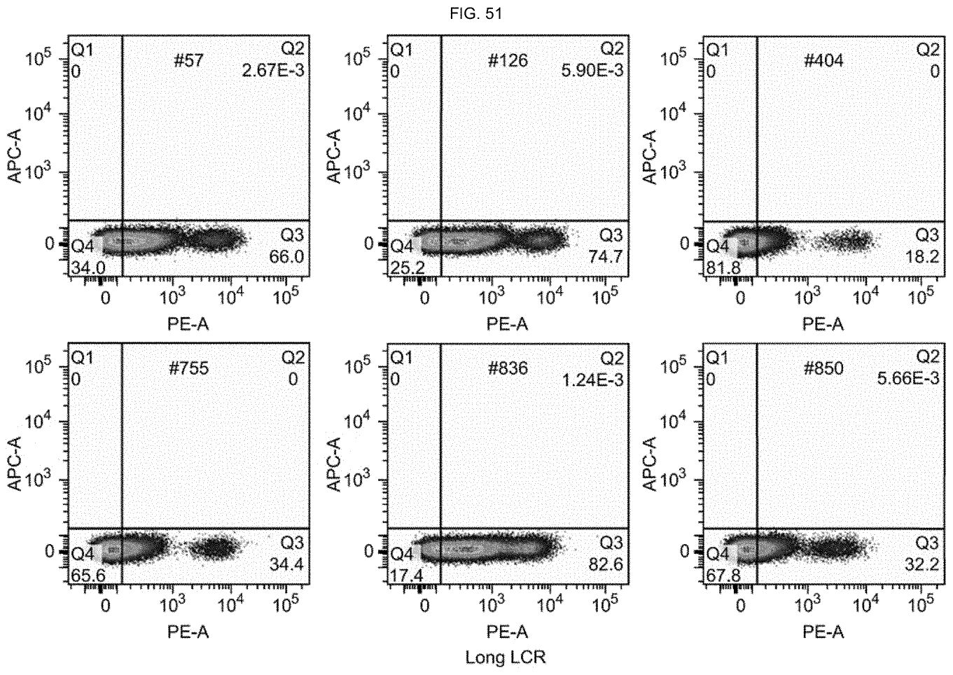

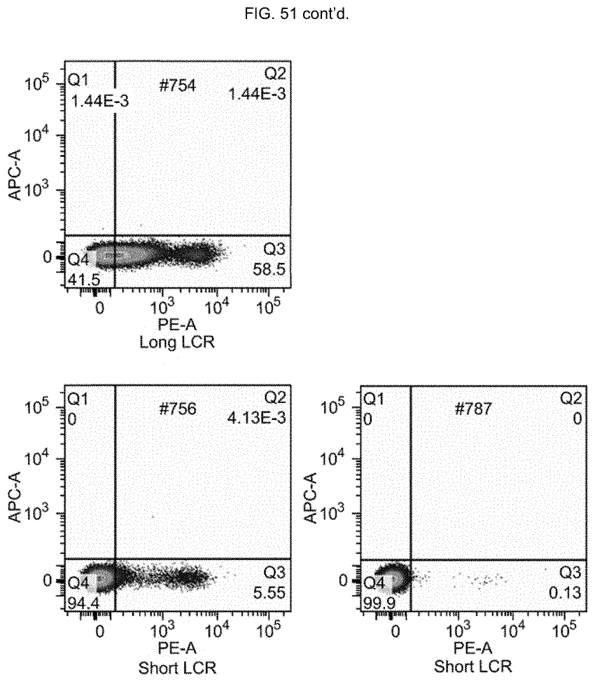

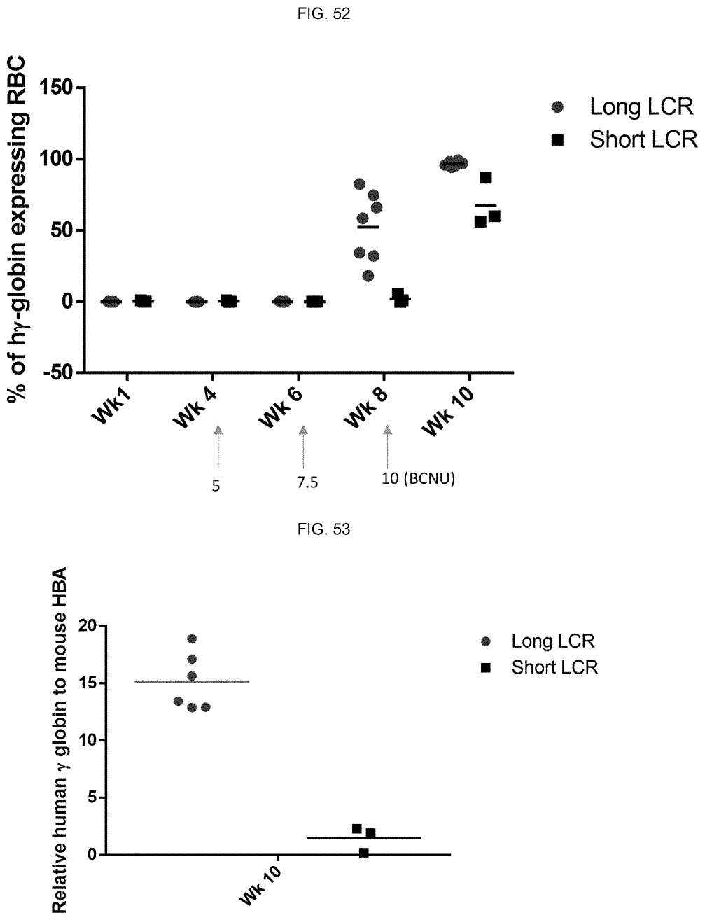

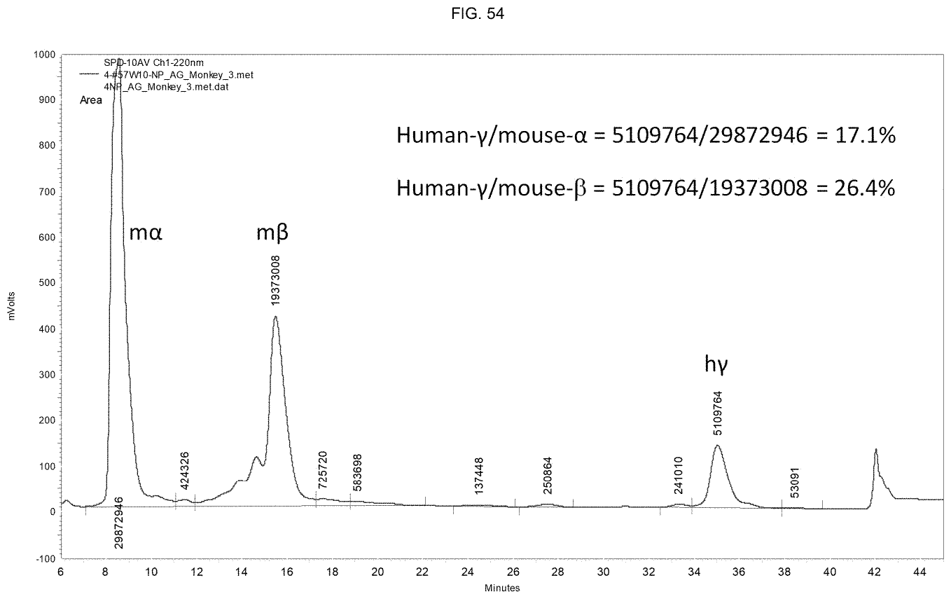

In thalassemic mice, 100% γ-globin marking in RBCs was achieved earlier and with fewer cycle of O⁶BG/BCNU in vivo selection of mice treated with HDAd-long-LCR compared to HDAd-short-LCR treated animals. This is important for the clinical translation of the approach. While the O⁶BG/BCNU in vivo selection system allows for a controlled increase of the percentage of γ-globin positive RBCs, it also causes transient leukopenia and side effects on the GI-tract (Wang et al, J Clin Invest. 129(2):598-615, 2019). A potential explanation for the requirement of less intense in vivo selection with HDAd-long-LCR could be that the long LCR prevents silencing of the EF1 a promoter driving the expression of the mgmt^{P140K} gene that provides resistance to O⁶BG/BCNU. This hypothesis is supported by the observation that mgmt mRNA levels (normalized to VCN) in bone marrow MNCs were significantly higher for HDAd-long-LCR (FIG. 48).

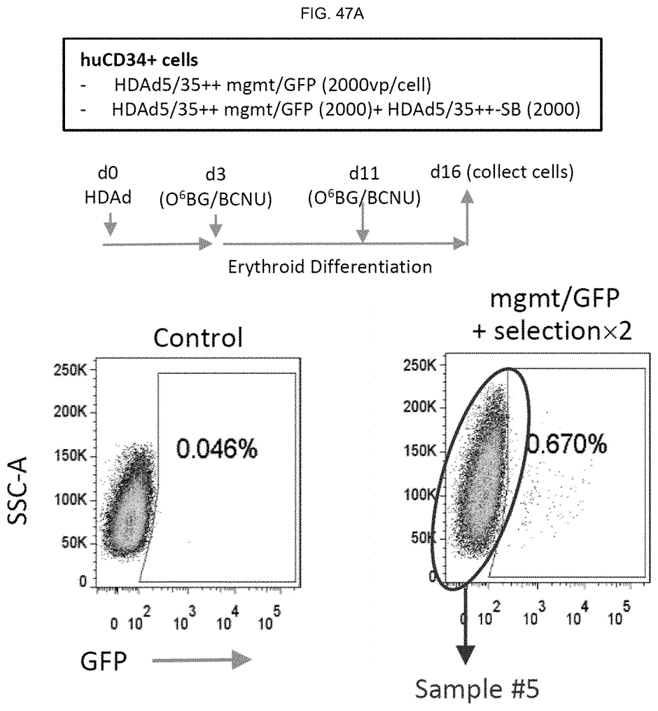

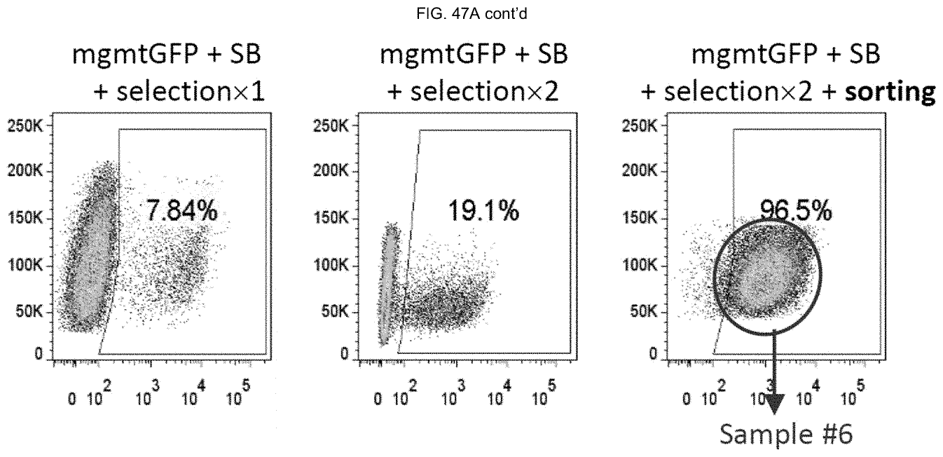

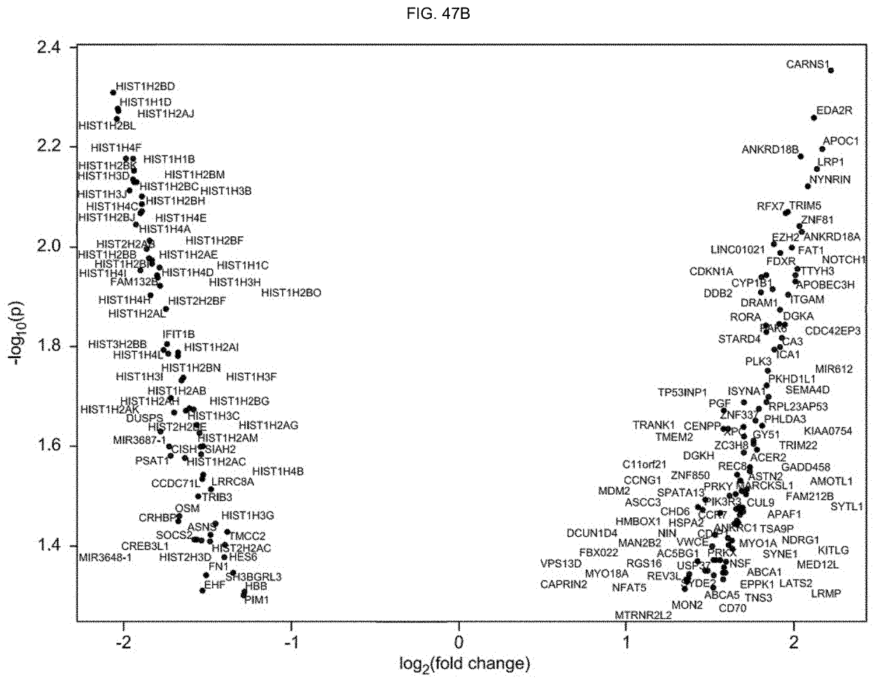

While this study focused on therapeutic aspects of the in vivo approach using HDAd-long-LCR, a number of mechanistic questions remain to be addressed in the future. One of these open questions is whether the long LCR prevents the transactivation of distant and neighboring genes. Furthermore, it is not completely clear whether the higher γ-globin expression levels from HDAd-long-LCR, which are also reflected at the mRNA level, are due to more active transcription initiation or less silencing of integrated vector copies, or both. The observation that in HDAd-long-LCR-treated Hbb^{th3}/CD46 mice, the percentage of γ-globin to mouse adult globin chains increased over time, a phenomenon that was also seen with in the CD46tg model in secondary recipients, could indicate that silencing, specifically in long-term repopulating cells, occurred over time and that the long-LCR protected against it. Higher mgmt^{P140K} mRNA levels per integrated vector copy (FIG. 48) also support the hypothesis that the long-LCR protects against silencing. To address these questions, future studies will focus on transduced CD34+ cell clones and will include genome-wide analysis using LAM-PCR/NGS (integration sites), chromosome conformation capture techniques, and RNA-Seq. A prerequisite for these studies would be that the SB100x transposase-mediated transgene integration and in vivo selection processes do not trigger undesired genomic alterations/rearrangements. In an attempt to assess this, RNA-Seq was performed on human CD34+ cells that stably expressed mgtm/GFP transgenes after SB100x-mediated integration and O⁶BG/BCNU selection in vitro (FIG. 47A). Modestly altered expression of only 176 genes was found, preferentially histone genes (FIG. 47B). This indicates that SB100x does not exert critical genotoxicity, which is also supported by the absence of clonal dominance in integration site analysis and the absence of hematological side effects in long-term studies.

The copy number of integrated transgenes analyzed in bone marrow MNCs 16 to 23 weeks after in vivo HSPC transduction/selection using the HDAd5/35++-based SB100x system was 2 copies per cell for transposons ranging from 13.8 (Wang et al., J Clin Invest. 129(2):598-615, 2019) to 32.4 kb. In order to form a catalytically primed transposon/transposase complex, the two ends of the transposon must be held together in close physical proximity by transposase molecules (Uchida et al., Nat Commun. 10(1):4479, 2019). This limitation has been addressed by incorporating frt sides into the HDAd vector which are recognized by the co-expressed Flpe recombinase leading to a circularization of the transposon (Turchiano et al., PLoS One. 9(11):e112712, 2014). The data reported here suggest that this process may make integration largely independent on the size of the transposon carried by HDAd5/35++ vectors.

This study demonstrates that using extended TAD/LCR core elements increases the expression level of a therapeutic transgene. While the β-globin LCR has been studied for decades, TAD core elements for other genes/clusters are less characterized. The median size of TAD is 880 kb. With further advancement of high-throughput chromosome conformation capture (3C) assay and its subsequent 4C, 5C and Hi-C protocols as well as fiber-Seq assays, the interrogation of the regulatory genome will progress at a rapid speed and, for gene therapy purposes, could deliver TADs that contain only critical core elements (Liu et al., BMC Genomics. 20(1):217, 2019).

In summary, the current Example shows that employing large regulatory elements in the context of HDAd5/35++ vectors for in vivo transduction of HSPCs in mice yielded a vector that confers γ-globin levels that meet gene expression thresholds thought to be curative for thalassemia major and Sickle Cell Anemia.

The human β-globin gene cluster lies in chromosome 11 and spans ˜100 kb. It has been proposed that the β-globin locus forms an erythroid-specific spatial structure composed of cis-regulatory elements and active β-globin genes, termed the active chromatin hub (ACH) (Tolhius et al., Mol Cell, 10:1453-1465, 2002). A core ACH is developmentally conserved and consists of the upstream 5′ DNAse hypersensitivity regions 1 to 5, called the globin LCR, and the downstream 3′HS1 as well as erythroid-specific transacting factors (Kim et al., Mol Cell Biol., 27:4551-65, 2007). For gene therapy applications, it is notable that a 23 kb β-globin LCR containing HS1 to HS5 plus a 3 kb 3′HS1 region conferred high-level, erythroid-specific, position independent expression upon cis-linked genes in transgenic mice (Grosveld, Cell, 51:975-985, 1987). A tool to deliver a transgene under the control of this LCR is available with 30+kb HDAd vectors.

The correction of many genetic diseases requires high level and tissue-restricted expression of the therapeutic gene, which can be accomplished by employing LCRs (Li et al., Blood 100: 3077-3086, 2002). For a cure of β-thalassemia major and Sickle Cell Anemia, it is thought that around 20% gene marking in HSPCs and 20% therapeutic-globin chain (β- or γ-globin) production in erythroid cells are required (Fitzhugh et al., Blood 130: 1946-1948, 2017). Due to size limitations, only truncated forms of the β-globin LCR can be used in lentivirus vectors which makes it difficult to meet the requirements for corrective gene expression levels (Uchida, et al., Nat Commun 10: 4479, 2019). A strategy to increase expression levels after lentivirus-mediated HSPC transduction is to increase the vector dose and thus the number of integrated transgene copies. This approach however enhances the risk of genotoxicity and tumorigenicity. Other attempts are focused on further optimizing globin expression cassettes (Uchida, et al., (2019) Nat Commun 10: 4479). HDAd vectors, having an insert capacity of 30 kb, are an ideal tool to develop the latter concept. In this Example, a HDAd5/35++ vector carrying a 29 kb γ-globin expression cassette was generated and tested after in vitro and in vivo HSPC transduction in CD46-transgenic mice.

In the HDAd vector system, the integration of the γ-globin cassette is mediated by the SB100x transposase. Non-viral gene transfer using the SB/transposon system is being used clinically for CD19 CAR T-cell therapy (Kebriaei et al., J Clin Invest 126: 3363-3376, 2016), age-related macular degeneration (Hudecek et al., Crit Rev Biochem Mol Biol 52: 355-380, 2017; Thumann et al., Mol Ther Nucleic Acids 6: 302-314, 2017), and Alzheimer's disease (Eyjolfsdottir et al., Alzheimers Res Ther 8: 30, 2016). HD-Ad mediated SB gene transfer was pioneered by the Kay and Ehrhardt groups. In their studies, transposons were relatively small; 4 kb-6 kb (Hausl et al., Mol Ther 18: 1896-1906, 2010; Yant et al., Nat Biotechnol 20: 999-1005, 2002). The current Example demonstrates for the first time that SB100x is capable of integrating a 32.4 kb transposon at an efficacy comparable to that of a 11.8 kb transposon, based on comparable VCNs (2-3 copies per cell). Per se this finding contradicts the observation that the efficacy of SB-mediated integration inversely correlates with the size of the SB transposon (Karsi et al., Mar Biotechnol (NY) 3: 241-245, 2001). The system appears to be lifted from the size limitation. First, in order to form a catalytically primed transposon/transposase complex, the two ends of the transposon must be held together in close physical proximity by transposase molecules (Hudecek et al., Crit Rev Biochem Mol Biol 52: 355-380, 2017). This limitation has been addressed by incorporating frt sides into the HDAd vector which are recognized by the co-expressed Flpe recombinase leading to a circularization of the transposon (Yant et al., Nat Biotechnol 20: 999-1005, 2002). The second mechanism limiting transposition of large constructs is a suicidal transpositional mechanism called auto-integration, i.e. the integration into TA dinucleotide inside the transposon (Wang et al., PLoS Genet 10: e1004103, 2014). The unseen differences in the VCN between HDAd-short-LCR and HDAd-long-LCR could be related to in vivo selection, which enriches for HSPCs and progenitors with a certain level of mgmt^{P140K} expression, i.e. for cells that have reached a threshold VCN.

Because of the powerful O⁶BG/BCNU in vivo selection system, nearly 100% of peripheral blood erythrocytes contained γ-globin. While this in vivo selection approach does not affect the cellular composition in the bone marrow, it results in leukopenia. Efforts are therefore focused on alternative approaches that do not involve the cytotoxic drug BCNU. Notably, as supported by the studies in the murine thalassemia model (Wang et al., J Clin Invest 129: 598-615, 2019), pharmaceutical in vivo selection might not be necessary in patients with hemoglobinopathies because gene-corrected HSPCs will have a proliferative advantage over non-corrected cells (Perumbeti et al., Blood 114: 1174-1185, 2009).

Given comparable VCNs for HDAd-short-LCR and HDAd-long-LCRs in primary animals and secondary recipients, γ-globin levels (measured by HPLC and qRT-PCR) in RBCs and bone marrow erythroid progenitors were significantly higher for the vector containing the long LCR. Interestingly, the differences between the two vectors were more pronounced in secondary recipients. This implies that RBCs that originated from transduced long-term repopulating HSPCs have higher γ-globin levels. Furthermore, HDAd-long-LCR displayed stronger erythroid specificity. These effects can be attributed to the additional LCR elements in HDAd-long-LCR that result in better access for transcription factors due to the LCR's chromatin opening ability (Li et al., Blood 100: 3077-3086, 2002), and/or the binding of additional transcription factors that result in increased transcription of the γ-globin gene. Another feature of the LCR is noteworthy, namely its ability to act as an autonomous regulatory unit, implying less transactivation of neighboring genes after random integration. In this context using a more complete LCR version decreases potential genotoxicity of the approach.

### Example 3. In Vivo HSC Gene Therapy Using a Combination of CRISPR-Triggered Reactivation of Endogenous Fetal Globin and SB100x Transposase-Mediated γ-Globin Gene Addition Cures Sickle Cell Disease in a Mouse Model

In patients with hereditary persistence of fetal globin and, more recently, in gene therapy patients, the degree of phenotypic correction of Sickle Cell Disease (SCD) correlates with the expression level of fetal γ-globin. It was recently reported that, after in vivo hematopoietic stem cell/progenitor (HSPC) transduction with HDAd5/35++ vectors, SB100x transposase-mediated γ-globin gene addition achieved 10-15% γ-globin of adult mouse globin, resulting in significant but incomplete phenotypic correction in a thalassemia intermedia mouse model. It has also been shown that genome editing of a γ-globin repressor binding site within the γ-globin promoter by CRISPR/Cas9 results in efficient reactivation of endogenous γ-globin. This example combines these two mechanisms to obtain curative levels of γ-globin after in vivo HSPC transduction.

A HDAd5/35++adenovirus vector (HDAd-combo) containing both modules was generated and tested in vitro and after in vivo HSPC transduction in “healthy” CD46/β-YAC mice and in a SCD mouse model (CD46/Townes), in which murine α- and β-globin genes were replaced with the human α-globin and human sickle β^{S}/fetal γ-globin genes. The present HDAd-combo contained a self-activating mechanism to reduce Cas9 expression after completion of target site cleavage. This resulted in significantly higher cleavage frequency in vivo, most likely due to better survival CRISPR/Cas9-edited HSPCs. Importantly, compared to HDAd vectors containing either the [symbol] -globin addition or the CRISPR/Cas9 reactivation units alone, significantly higher [symbol] -globin was found in RBCs after transduction with HDAd-combo. At week 13 after in vivo HSC transduction of CD46/Townes mice with the combo vector, the level of [symbol] -globin level in red blood cells was 30% of that of adult human α- and β^{S}-chains. This resulted in a complete phenotypic correction of SCD.

Introduction:

SCD gene therapy: Sickle Cell Disease and β-thalassemia are the most common monogenic disorders worldwide, with 317,000 affected neonates born each year. SCD is caused by a single mutation on the first exon of b-globin gene (β^{S} allele), resulting in the formation of defective hemoglobin tetramers, which polymerize upon low oxygen concentrations, leading to destruction of erythrocytes. SCD is associated with substantial morbidity, poor quality of life, and a shortened life expectancy. The clinical course of SCD is improved when fetal [symbol] -globin genes are highly expressed as seen in patients with HPFH traits (Conley et al., Blood 21: 261-281, 1963; Stamatoyannopoulos et al., Blood 46: 683-692, 1975). In SCD, [symbol] -globin exerts a potent anti-sickling function by competing with the sickle β-globin for incorporation in the Hb tetramers and by inhibiting sickle hemoglobin (HbS) polymerization. Pharmacological treatments increasing HbF levels are not equally effective in all patients. The development of gene therapy for β-hemoglobinopathies has been justified by the limited availability of matching donors and the narrow window of application of HSPC transplantation to the youngest patients. Current SCD gene therapy approaches involve the collection of HSPCs, their in vitro culture, transduction with lentivirus vectors carrying either an intact β-globin, an anti-sickling β-globin or, a fetal [symbol] -globin expression cassette, and retransplantation into myelo-conditioned patients. Phase I gene therapy trials with γ-globin gene addition lentivirus vectors are promising, however a long-term cure of all the SCD symptoms has so far not been achieved (Demirci et al., Hum Mol Genet., 2020. doi: 10.1093/hmg/ddaa088). For a cure of the disease, [symbol] -globin levels in RBCs must be at least 20% of adult α-globin, and optimally, β^{S} levels should be reduced. This is difficult to achieve with lenti-virus vectors, because of insert size limitations preventing the use of full-length globin LCRs or multi-modality genome editing cassettes (Uchida et al., Nat Commun 10: 4479, 2019).

In vivo HSPC gene therapy- [symbol] -globin gene addition: A major risk of ex vivo HSPC gene therapy is transplant-related morbidity (Anurathapan et al., Biol Blood Marrow Transplant 20: 2066-2071, 2014; Lucarelli et al., Blood Rev 16: 81-85, 2002; Storb et al., Hematology Am Soc Hematol Educ Program: 372-397, 2003). Furthermore, the use of lentivirus vectors bears the risk that transgene expression is silenced or chromosomal proto-oncogenes are activated. Importantly, the approach is complex, expensive, and therefore difficult to perform in countries with limited resources where SCD is prevalent. A simple in vivo HSPC gene therapy approach has been developed. It involves the subcutaneous injection of GCSF/AMD3100 to mobilize HSPCs from the bone marrow into the peripheral blood stream and the intravenous injection of an integrating helper-dependent adenovirus vector system, HDAd5/35++ vectors. These vectors have an insert capacity of 30+kb and target CD46, a receptor that is expressed on primitive HSPCs (Richter et al, Blood 128: 2206-2217, 2016). Innate toxicity associated with intravenous HDAd5/35++injection can be controlled by glucocorticoid, IL6- and IL1β-receptor antagonist pretreatment in mice and in non-human primates (Li et al., 23rd Annual ASGCT meeting. 2020; abstract #546) Random transgene integration is mediated by an activity-enhanced Sleeping Beauty transposase (SB100x) (Boehme et al., Mol Ther Nucleic Acids 5: e337, 2016). In this system, the transgene cassette is flanked by inverted repeats (IRs), which are recognized by the SB100x transposase and frt sites that allow for circularization of the transgene cassette in the presence of Flp recombinase. The second vector, HDAd-SB, supplies Flp recombinase and SB100x in trans to mediate integration of the GFP cassette into a TA dinucleotide of the genomic DNA (Mates et al., Nat Genet 41: 753-761, 2009). In a previous study with HDAd5/35++ vectors, a 4.3 kb HS1-HS4 mini-LCR (β-globin locus control region) was used in combination with a 0.66 kb β-globin promoter to drive human [symbol] -globin expression after in vivo HSPC transduction (Wang et al., J Clin Invest 129: 598-615, 2019; Li et al., Mol Ther Methods Clin Dev 9: 142-152, 2018). In Hbb^{th3}/CD46+/+ thalassemic mice, stable (8+months) [symbol] -globin marking was achieved in nearly 100% of peripheral blood erythrocytes and near complete phenotypic correction (Wang et al., J Clin Invest 129: 598-615, 2019). However, the level of [symbol] -globin expression was only 10-15% of that of adult mouse α-globin with an average integrated vector copy number (VCN) of 2 copies per cell, thus rendering the clinical translation of the approach to SCD particularly challenging.

In vivo HSPC gene therapy—reactivation of endogenous [symbol] -globin: In hereditary persistence of fetal hemoglobin (HPFH), a benign genetic condition, mutations attenuate γ-to-β globin switching, causing high fetal globin (HbF) levels throughout life thus alleviating the clinical manifestations of these disorders (Forget, Ann NY Acad Sci 850: 38-44, 1998). Early studies attempted to re-enact HPFH mutations by either creating large deletions within the β-globin locus (Sankaran, Hematology Am Soc Hematol Educ Program 2011: 459-465, 2011), or by introduction of mutations in the HBG promoters can increase the levels of HbF in erythroid cells (Wienert et al., Nat Commun 6: 7085, 2015; Traxler et al, Nat Med 22: 987-990, 2016; Lin et al., Blood 130: 284, 2017). With the discovery of BCL11A as a fetal globin repressor, these attempts became more focused involving the targeted disruption of the BCL11A binding site within the HBG promoters (Masuda et al., Science 351: 285-289, 2016) or the disruption of the erythroid bcl11a enhancer to reduce BCL11A expression (Wu et al., Nat Med 25: 776-783, 2019) by either CRISPR/Cas9 or, recently, base editors (Zeng et al., Nat Med 26: 535-541, 2020). An HBG1/HBG2 promoter targeted CRISPR/Cas9 was employed to reactivate γ-globin in human β-globin locus-transgenic (β-YAC) mice (Li et al., Blood 131: 2915-2928, 2018). After in vivo HSPC transduction, efficient target site disruption resulting in a pronounced switch from human β- to [symbol] -globin expression in red blood cells of adult mice that was maintained after secondary transplantation of HSPCs was demonstrated. In long-term follow up studies, hematological abnormalities were not detected, indicating that HBG promoter editing does not negatively affect hematopoiesis.

It was previously reported that expression of CRISPR/Cas9 from HDAd5/35++ vectors can compromise the stem cell function and survival of transduced HSPCs, specifically human HSPCs (Li et al., Mol Ther Methods Clin Dev 9: 390-401, 2018). Therefore, approaches to shorten CRISPR/Cas9 expression were developed (Li et al., Mol Ther Methods Clin Dev 9: 390-401, 2018; Li et al., Mol Ther 27: 2195-2212, 2019).

Here, the aim was to achieve curative levels of γ-globin after in vivo HSPC transduction by combining SB100x-mediated [symbol] -globin gene addition and reactivation of [symbol] -globin in β-YAC mice as well as in mouse model of Sickle Cell disease developed by Tim Townes (hα/hα::β^{S}/β^{S}) (Wu et al., Blood 108: 1183-1188, 2006). In this model, the murine α-globin genes were replaced with the human α-globin and the murine adult β-globin genes were replaced with human sickle β^{S} and fetal γ-globin genes linked together. This model displays key phenotypic features of Sickle Cell Disease.

Materials and Methods

Reagents: G-CSF (Neupogen™) (Amgen Thousand Oaks, Calif.) and AMD3100 (Sigma-Aldrich, St. Louis, Mo.) were used. O⁶-BG and BCNU were from Sigma-Aldrich (St, Louis, Mo.).

HDAd vectors: The HDAd-CRISPR (“cut”), HDAd-SB-addition (“add”) and HDAd-SB have been described previously (Li et al., Blood 131(26):2915-2928, 2018; Wang et al., J Clin Invest 129: 598-615, 2019). The cloning of pHCA-Combo involved 3 steps. Step 1) The sgHBG #2 (SEQ ID NO: 258) targeting the BCL11A binding site in HBG1/2 promoter regions was synthesized, annealed and inserted into the BbsI site of pSPgRNA (Addgene, Cambridge, Mass.), generating pSP-sgHBG #2. The 0.4 kb U6-sgHBG #2 fragment in pSP-sgHBG #2 was amplified and cloned to the BamHI site of pBST-sgAAVS1-miR (Li et al., Mol Ther 27: 2195-2212, 2019), obtaining pBST-sgHBG #2-miR. Step 2) A 1.5 kb PGK-mgmt-bGHpolyA fragment was synthesized as gBlock (IDT, Newark, N.J.) and ligated with ClaI-digested pBS-LCR-globin-mgmt (Li et al., Mol Ther 27: 2195-2212, 2019), getting pBS-LCR-globin-PGK-mgmt. Next, a 4.8 kb sequence containing the pBS-Frt-IR region was amplified from pBS-FRT-IR-Ef1α-mgmt (Li et al., Cancer Res 80: 549-560, 2020) and ligated with EcoRV-KpnI digested pBS-LCR-globin-PGK-mgmt, leading to pBS-Frt-IR-LCR-globin-PGK-mgmt. In this step primers containing 15 bp homology arms (HAs) for later infusion cloning (Takara, Mountain View, Calif.) were used. The two 15 bp HAs flanking the two Frt-IR components can be exposed upon PacI digestion to facilitate recombination with the modified pHCA construct described below. Step 3) The 5.3 kb XbaI fragment of pHCAS1S-MCS (Li et al., Mol Ther 27: 2195-2212, 2019) was deleted by XbaI restriction and re-ligation, generating pHCAS1S1-MCS. A 7.6 kb CRISPR cassette starting from the U6 promoter to the SV40 polyA signal sequence was amplified from pBST-sgHBG #2-miR and cloned to the NheI site of pHCAS1S1-MCS, forming pHCAS1S1-MCS-sgHBG #2. Lastly, the 12.0 kb HA-flanked Globin/mgmt cassette in pBS-Frt-IR-LCR-globin-PGK-mgmt was released by PacI treatment and recombined with PacI-digested pHCAS1S1-MCS-sgHBG #2, resulting in pHCA-Combo. The final construct was screened by several restriction enzymes (HindIII, EcoRI and PmeI) and confirmed by sequencing the whole region containing transgenes.

For the production of HDAd5/35++ vectors, corresponding plasmids were linearized with PmeI and rescued in 116 cells (Palmer & Ng, Mol Ther 8: 846-852, 2003) with AdNG163-5/35++, an Ad5/35++ helper vector containing chimeric fibers composed of the Ad5 fiber tail, the Ad35 fiber shaft, and the affinity-enhanced Ad35++fiber knob (Richter et al., Blood 128: 2206-2217, 2016). HD-Ad5/35++ vectors were amplified in 116 cells as described in detail elsewhere (Palmer & Ng, Mol Ther 8: 846-852, 2003). Helper virus contamination levels were found to be <0.05%. Titers were 2-5×10¹² vp/ml.

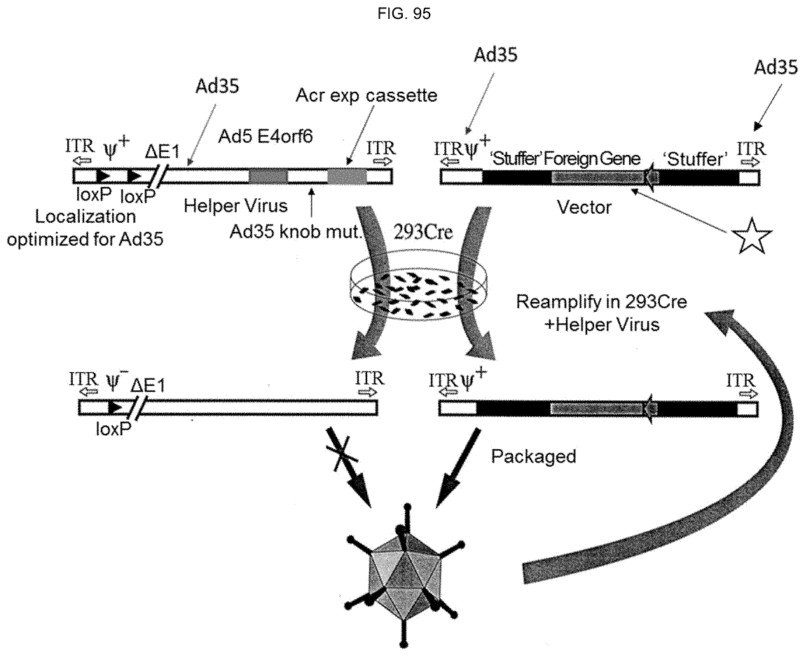

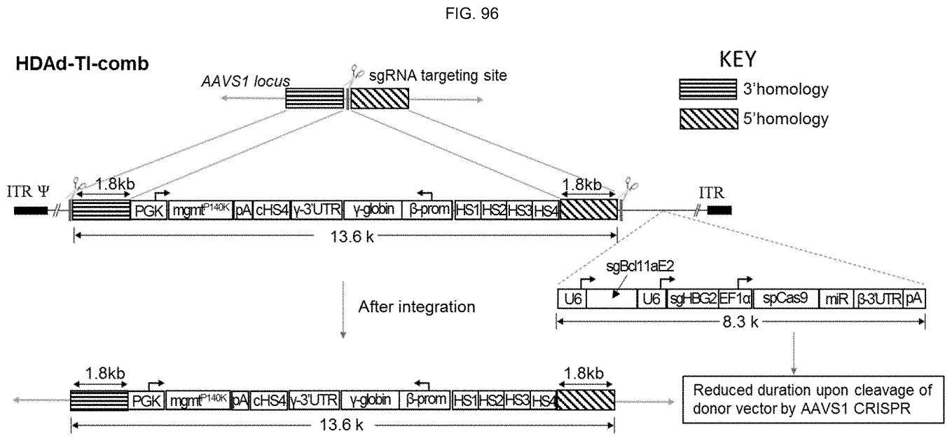

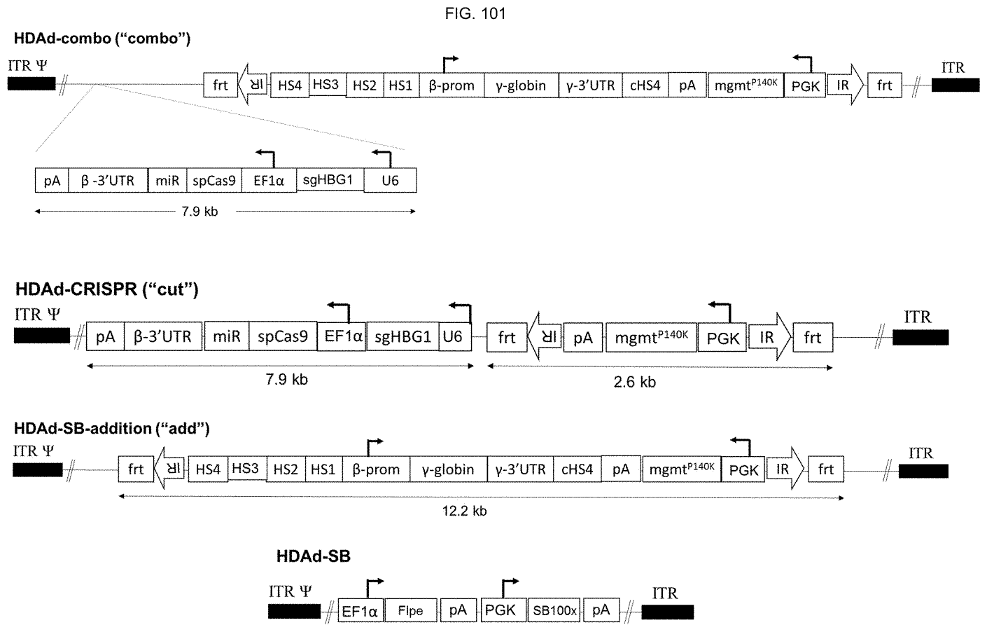

Vectors of the present Example are illustrated in FIG. 101, and include an HDAd combination adenoviral vector that includes both (i) a nucleic acid encoding a γ-globin transgene (“addition”) present in a transposon and (ii) a nucleic acid encoding a CRISPR/Cas9 system targeting HBG1/2 (“CRISPR”) for increased expression of endogenous γ-globin, not present in the transposon (the two together form the “Combination”). For further disclosure in relation to dual vectors, see also FIGS. 96, 102, 97A-97D, 98A-98N, 99A-99U).

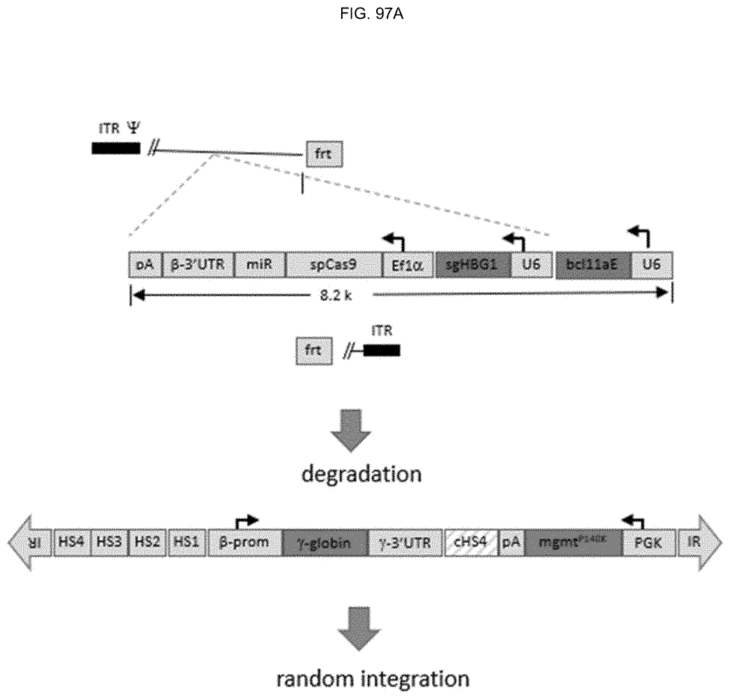

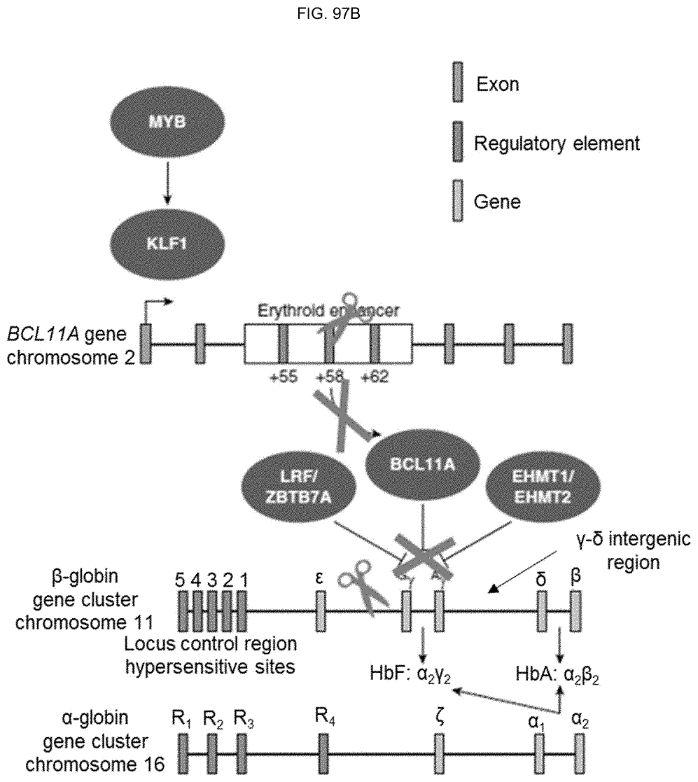

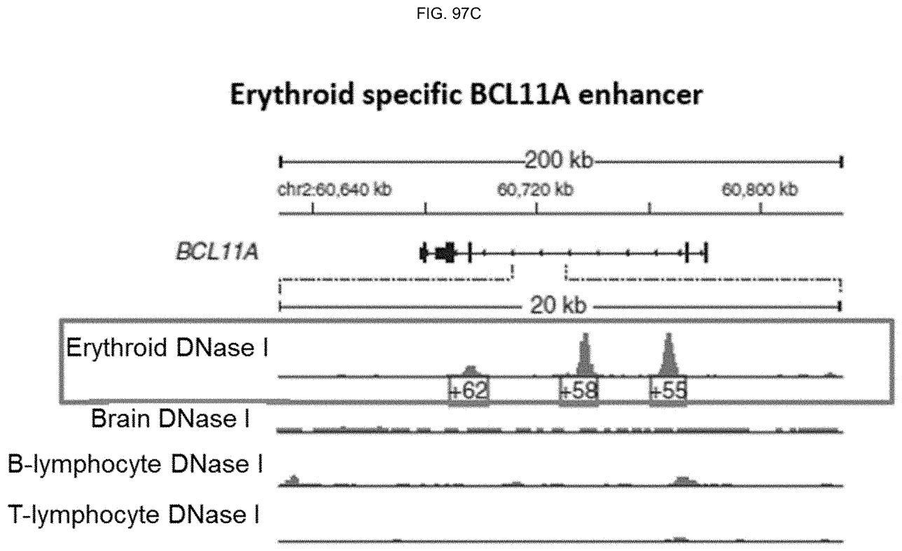

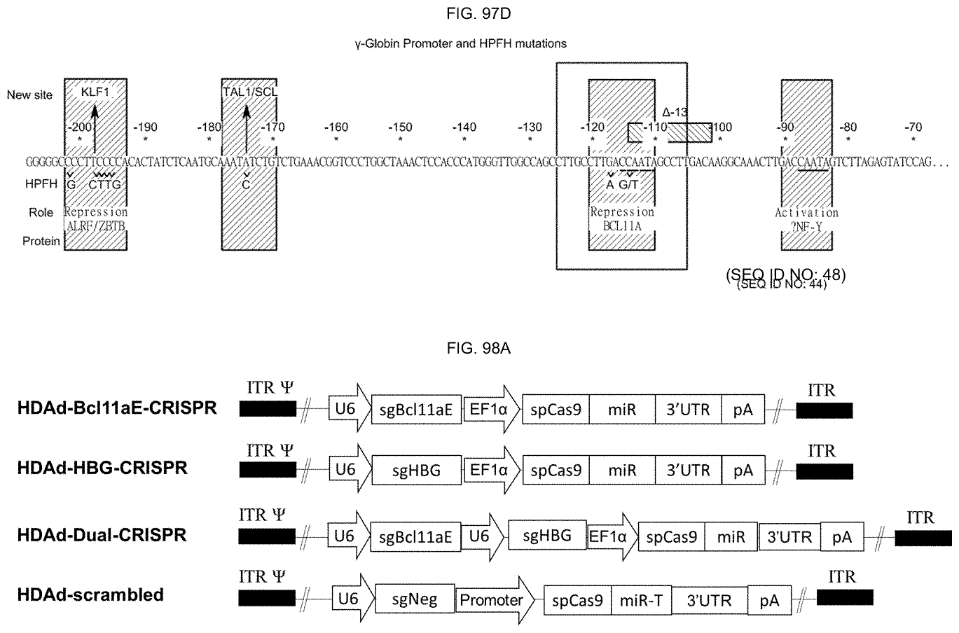

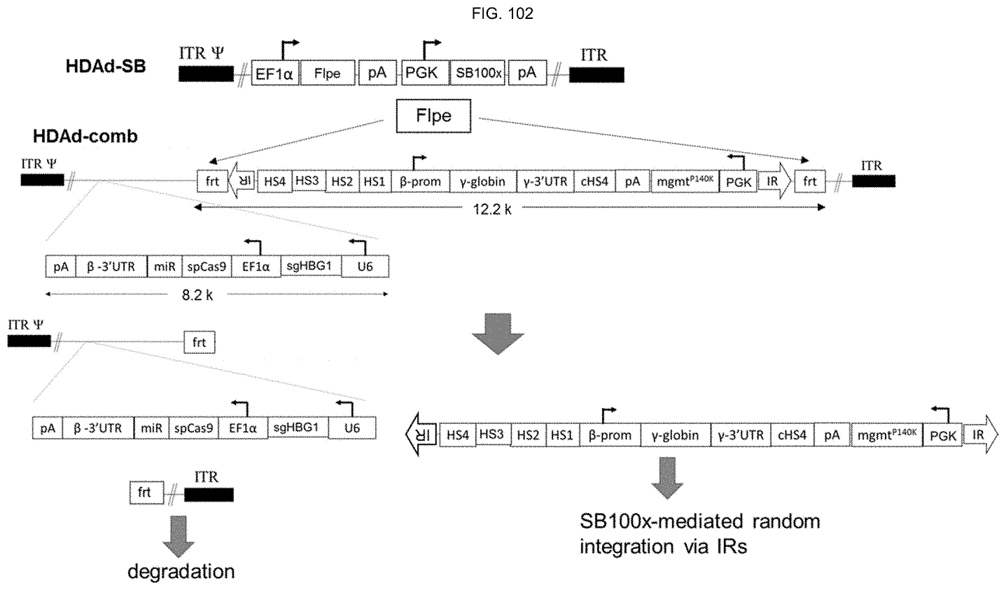

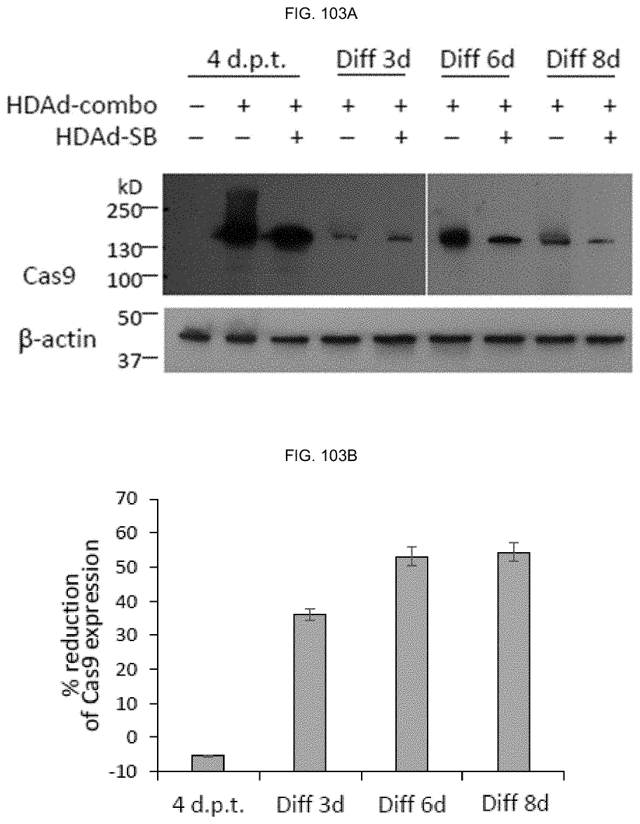

Specifically, FIG. 96 shows the schematic of HDAd-Tl-combo vector in which the CRISPR system targets two different sites (HBG promoter and erythroid bcl11a enhancer), which leads to increased gamma reactivation. FIG. 102 shows how, in HDAd-combo, the interaction of Flpe recombinase with the frt sites leads to a circularization of the transposon, leaving linear fragment of the vector containing the CRISPR cassette. Previous studies with the SB100x/Flpe system demonstrated that these vector parts are rapidly lost while the circularized transposon is integrated into the host genome by SB100x (Yant et al., Nat Biotechnol., 20: 999-1005, 2002). FIG. 97A shows how upon co-infection of HDAd-SB and HDAd-combo, Flpe will be expressed and release the IR-flanked transposon, which will then be integrated into the genome by SB100x transposase. Simultaneously, HBG1 and bcl11a-E CRISPRs will be expressed and generate DNA indels that will lead to reactivation of γ-globin. Upon Flp-mediated release of the transposon, the CRISPR cassette will be degraded, thereby avoiding cytotoxicity. The CRISPR system targets two different sites (HBG promoter and erythroid bcl11a enhancer), which leads to increased γ reactivation. The targeting strategy (FIG. 97B), erythroid specific BCL11A enhancer (FIG. 97C), and BCL11A binding site at HBG promoter (FIG. 97D) are also shown.

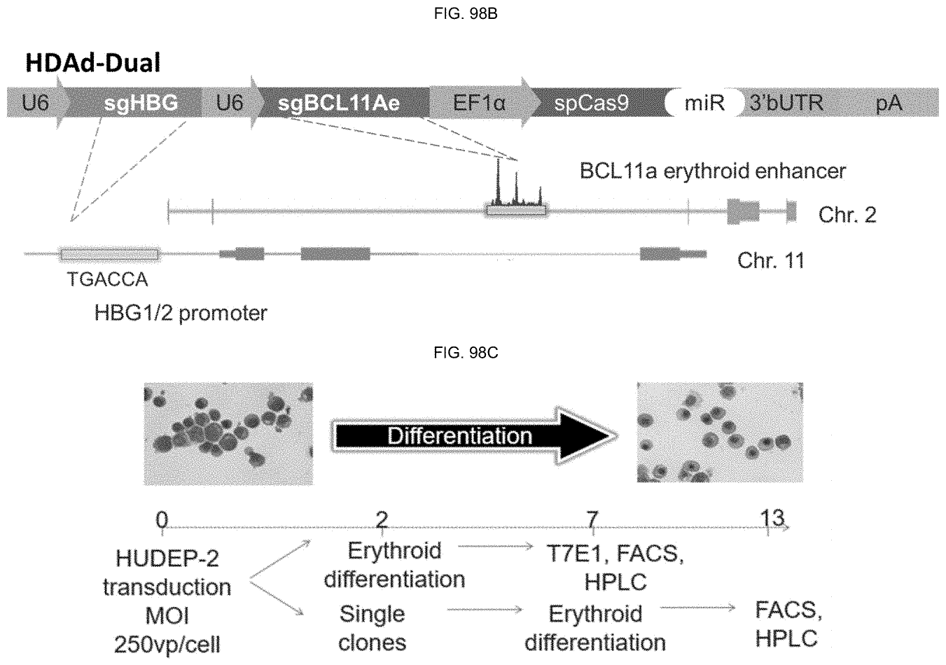

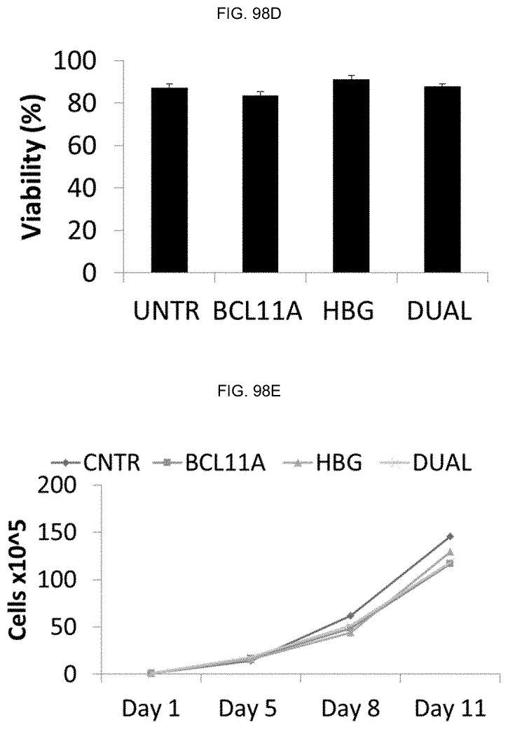

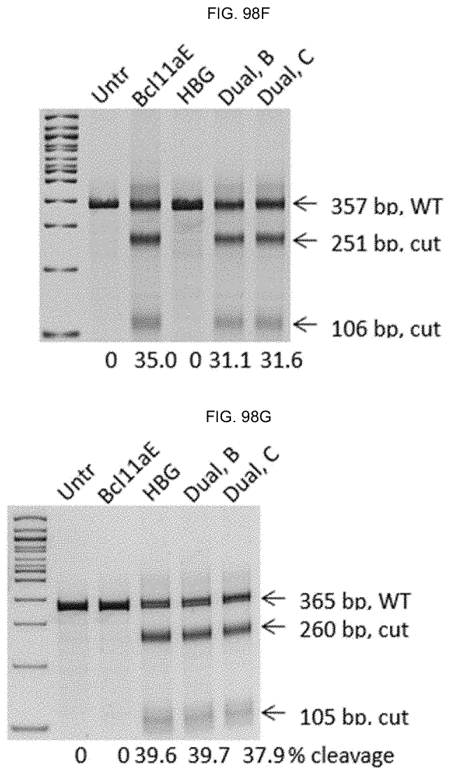

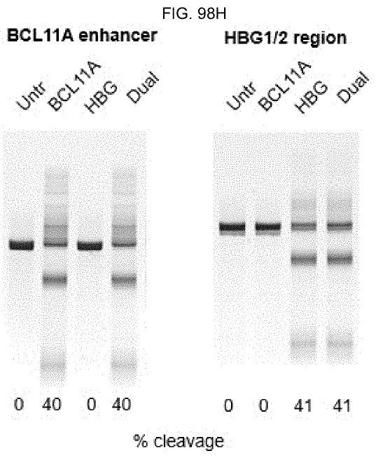

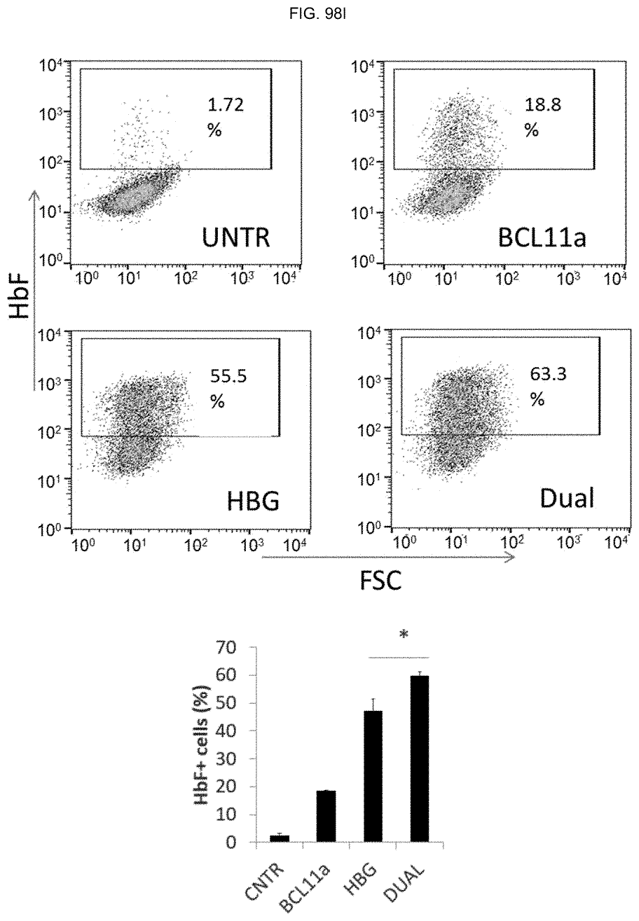

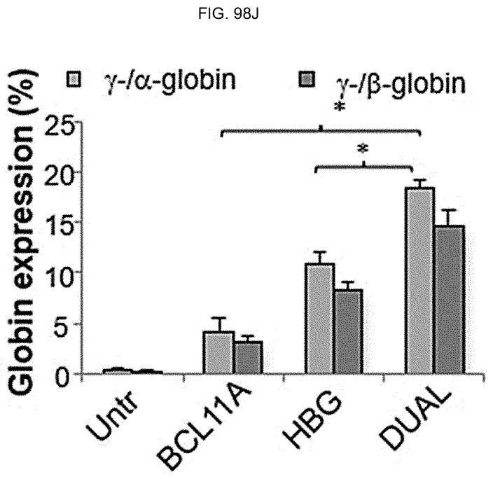

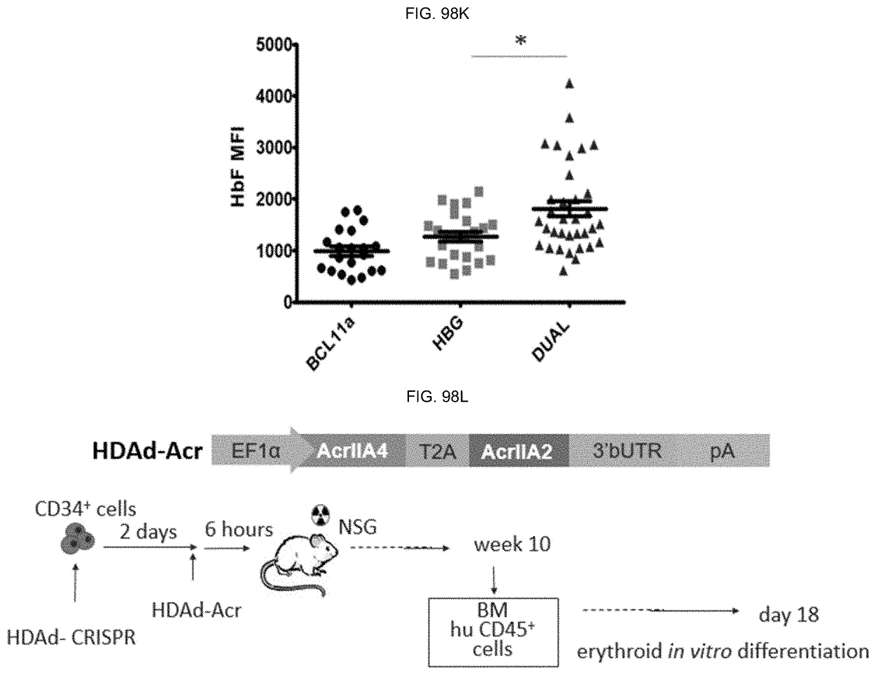

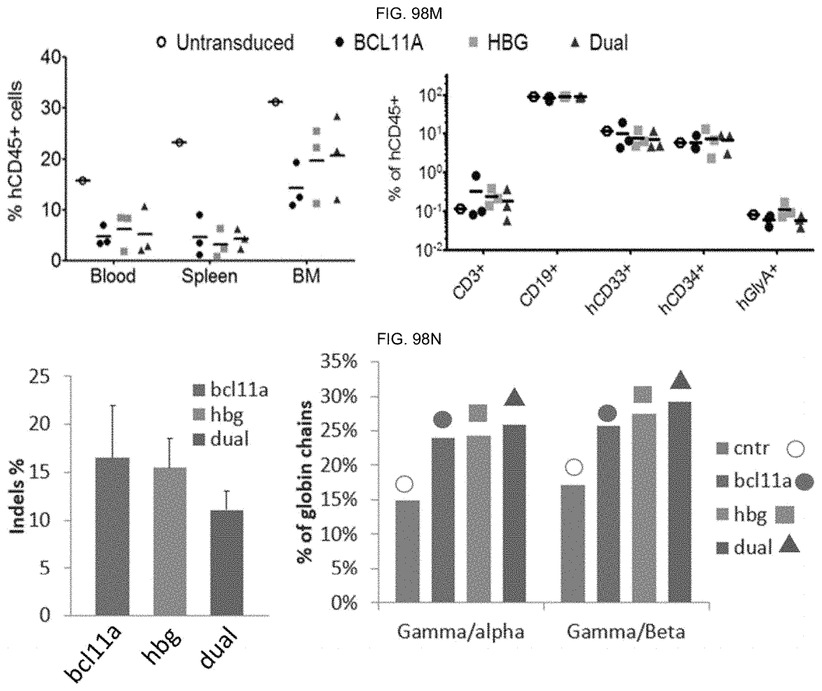

Dual CRISPR vectors and [symbol] -globin reactivation are shown in FIGS. 98A-98N. The vector designs for HDAd-Bcl11ae-CRISPR, HDad-HBG-CRISPR, HDAd-Dual-CRISPR, HDAd-scrambled (FIG. 98A) and HD-Ad5/35++CRISPR Vectors for dual gRNA vector (FIG. 98B) are shown. HD-Ad5/35++CRISPR transduction of a human erythroid progenitor cell line (HUDEP-2) is shown before and after differentiation in FIG. 98C. The HD-AD5/35++“Dual” gRNA vector does not negatively affect cell viability (FIG. 98D) nor proliferation (FIG. 98E) compared to untreated (UNTR), BCL11A, or HBG vectors. The Dual vectors achieve similar editing levels similar to those observed with the single gRNA vectors for the target loci (FIG. 98F) Bcl11a enhancer and (FIG. 98G) HBG promoter. Furthermore, the HD-AD5/35++“Dual” gRNA vector achieves editing levels of target loci similar to those observed with the single gRNA vectors (FIG. 98H). A significantly higher percentage of HbF+ cells were observed by flow cytometry in HUDEP-2 cells transduced with the HD-Ad5/35 “Dual” gRNA vector compared to the single gRNA vectors (FIG. 98I). The overall gamma globin expression, measured by HPLC, was significantly higher in the dual targeted samples (FIG. 98J). A significantly higher fetal globin expression in double knock-out clones than single knock-out clones was observed implying a possible synergistic effect of the two mutations, leading to higher gamma expression/cell (FIG. 98K). FIG. 98L shows that peripheral blood mobilized CD34+ cells were transduced with the HDAd5/35++ CRISPR vectors. To minimize CRISPR/Cas9 cytotoxicity, cells were subsequently transduced with an HDAd5/35++ vector that expresses anti-Cas9 peptides. Cells were transplanted into sub-lethally irradiated NSG mice and analyzed. At week 10 after transplantation, cells transduced with the HD-Ad5/35 “Dual” gRNA vector exhibited similar engraftment to the cells transduced with the single gRNA vectors. Lineage composition was similar in all groups (FIG. 98M). CD34+ cells transduced and edited by the double gRNA vector, efficiently engrafted in NSG mice (FIG. 98N). Furthermore, the engrafted dual targeted cells after erythroid differentiation expressed higher levels of gamma globin to the control, compared to the single targeted cells, despite the relatively lower editing levels (FIG. 98N).

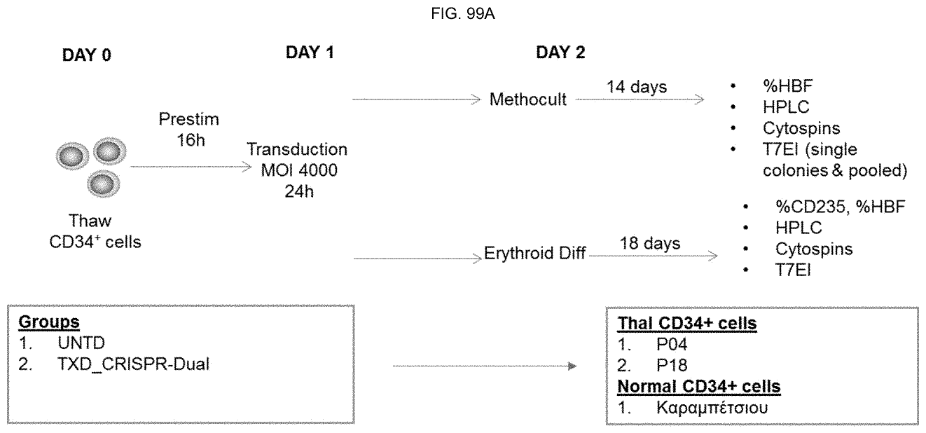

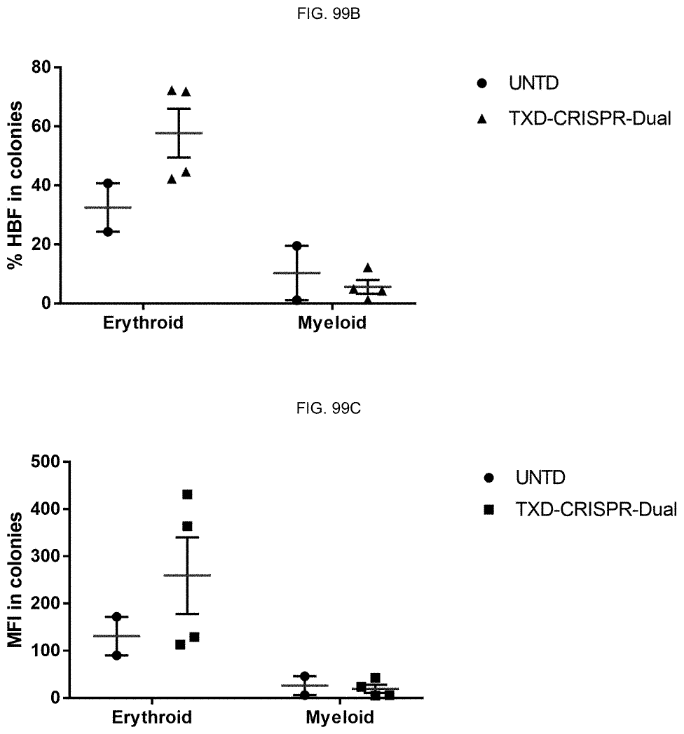

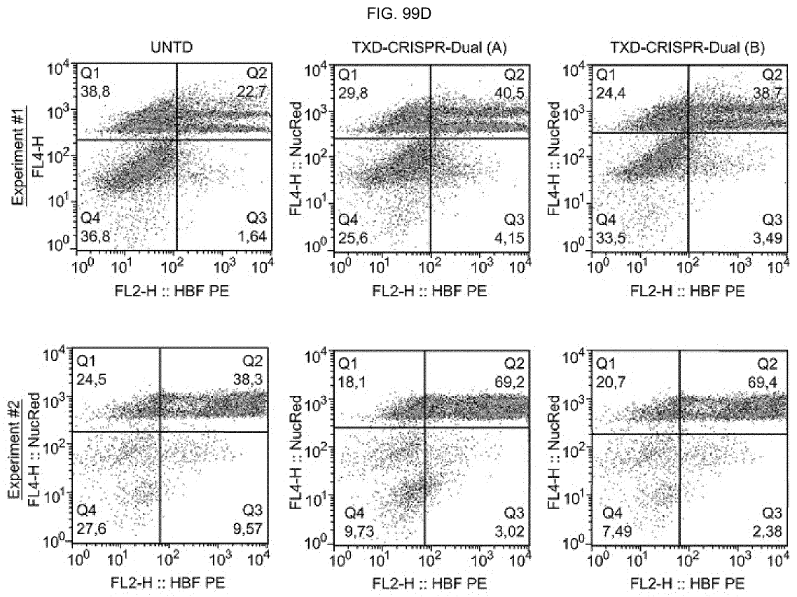

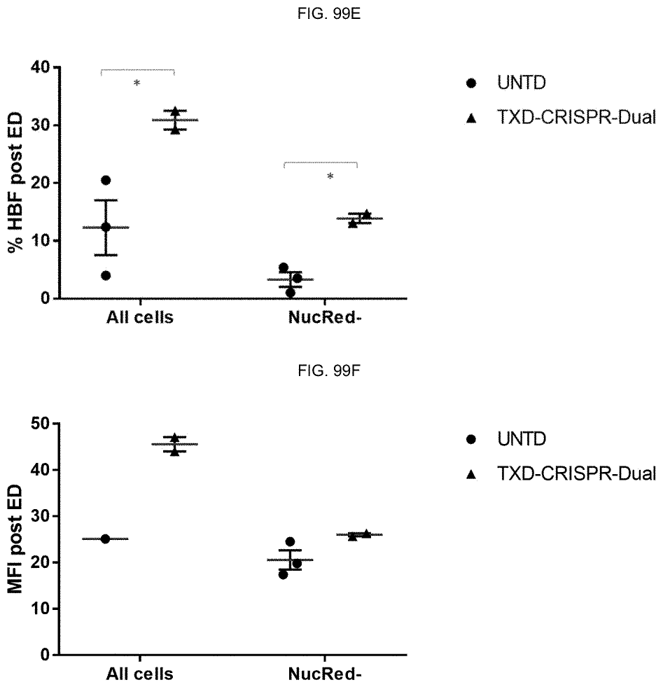

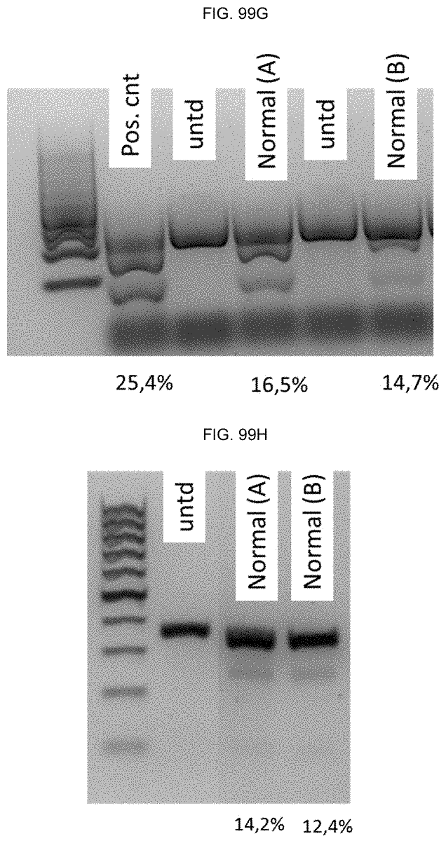

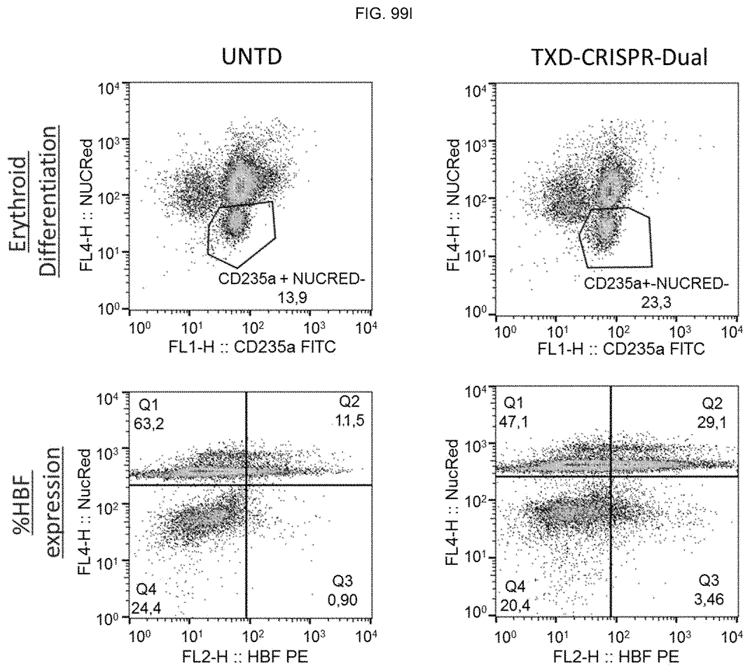

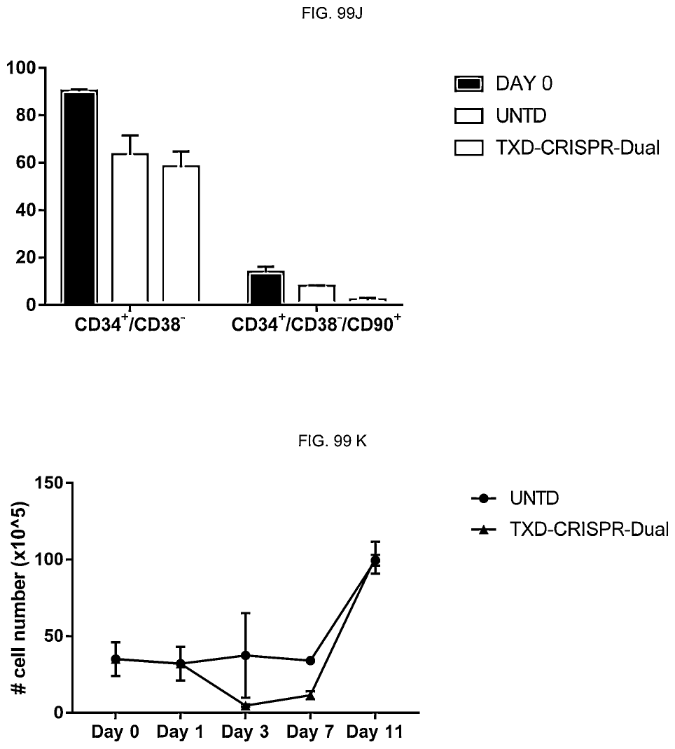

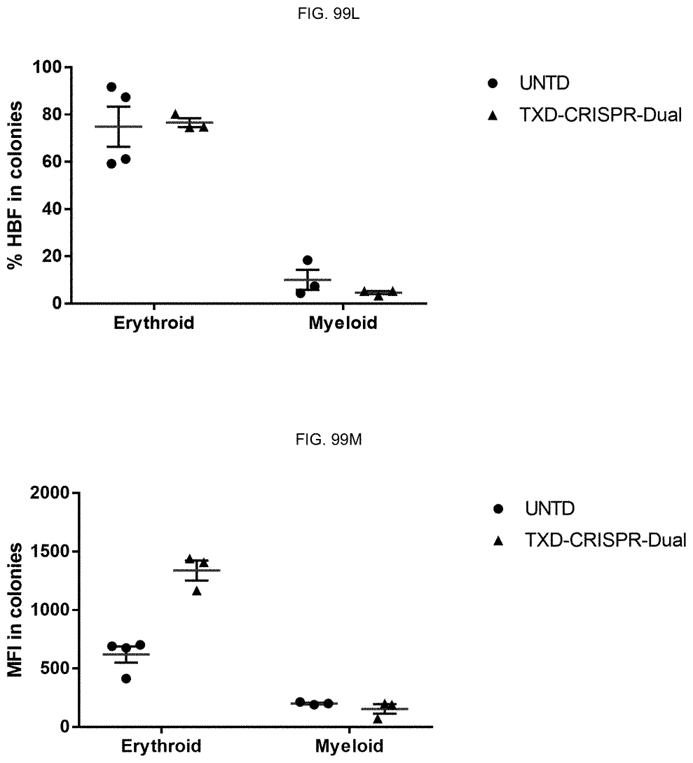

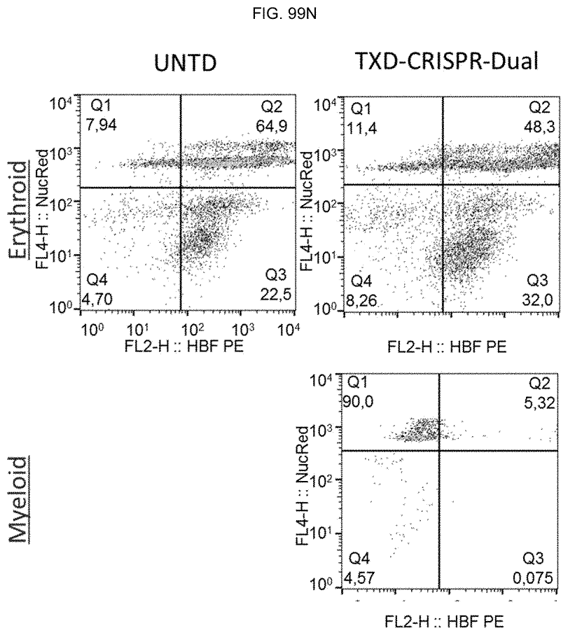

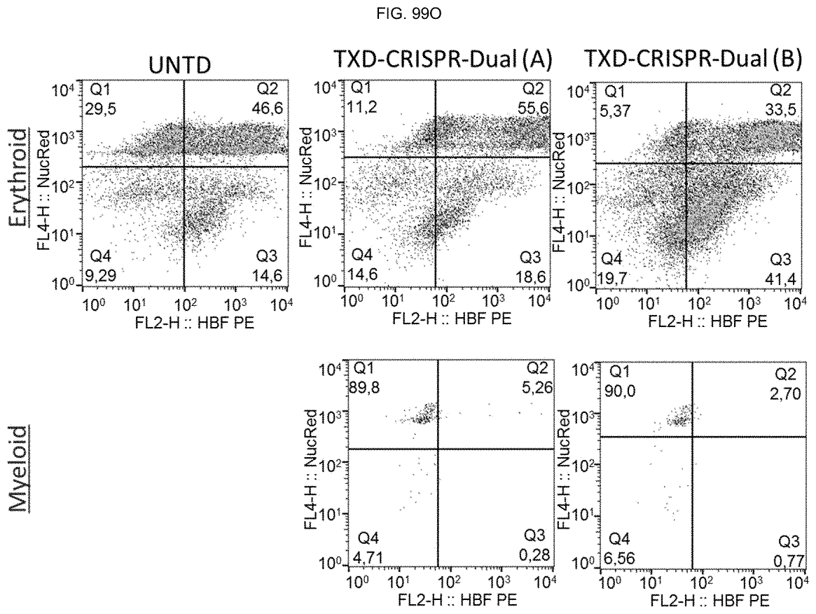

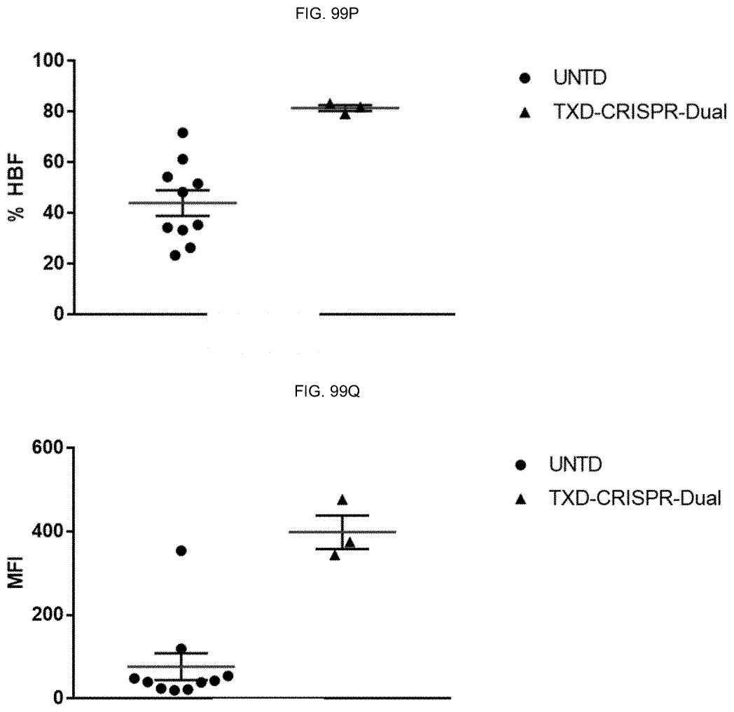

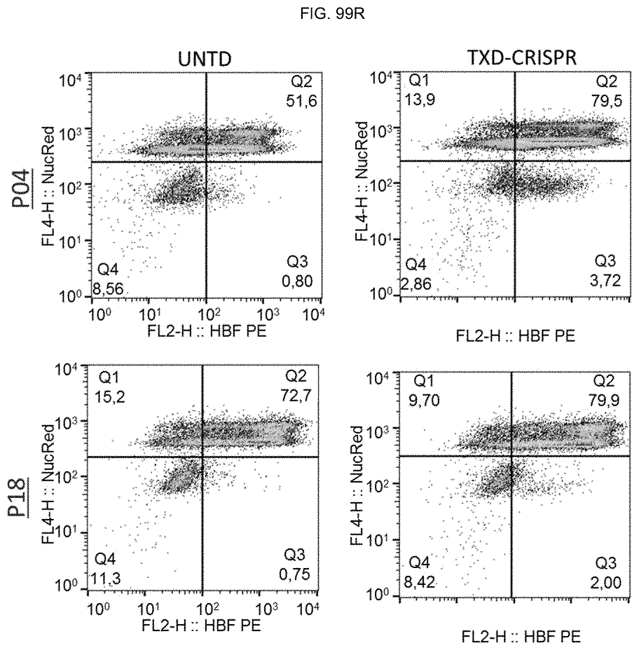

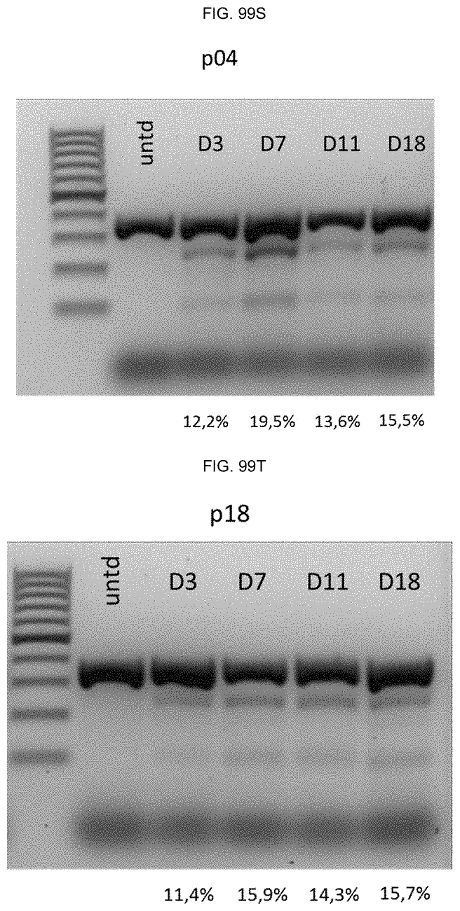

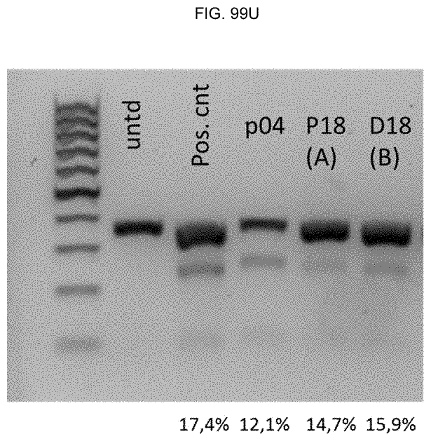

The experimental design for the ex vivo transduction of double edited normal and that CD34+ cells is shown in FIG. 99A. HBF expression (FIG. 99B), MFI (FIG. 99C), and flow cytometry data describing HBF expression (FIG. 99D) in colonies on day 15 for normal CD34+ cells are shown. HBF expression (FIG. 99E) and MFI (FIG. 99F) after erythroid differentiation (ED) for normal CD34+ cells are shown. TE71 for HBG site (FIG. 99G) and TE71 for BCL11A site (FIG. 99H) are shown 48 hours post transduction (txd) in normal CD34+ cells. Flow cytometry data describing HBF expression in EC and erythroid differentiation can be found in FIG. 99I. FIGS. 99J-99U show results in ThaI CD34+ cells. The immunophenotype of cells at day 0, untransduced cells and cells transduced with CRISPR-Dual (FIG. 99J) and a growth curve comparing untransduced cells and cells transduced with CRISPR-Dual (FIG. 99K) over 11 days. HBF expression (FIG. 99L) and MFI (FIG. 99M) are shown in colonies on day 15. HBF expression in EC (FIG. 99P), MFI (FIG. 99Q), and flow cytometry data describing HBF expression and PO4 and P18 (FIG. 99R) are also shown. TE71 for HBG site erythroid differentiation at p04 (FIG. 99S) and p18 (FIG. 99T) are shown while FIG. 99U shows TE71 for the BCL11A site 48 hours after transduction.

HUDEP-2 cells/erythroid differentiation: HUDEP-2 cells (Kurita et al., PLoS One 8: e59890, 2013) were cultured in StemSpan SFEM medium (STEMCELL Technologies) supplemented with 100 ng/ml SCF, 3 IU/ml EPO, 10⁻⁶ M dexamethasone and 1 μg/ml doxycycline (DOX). Erythroid differentiation was induced in IMDM containing 5% human AB serum, 100 ng/ml SCF, 3 IU/ml EPO, 10 μg/ml Insulin, 330 μg/ml transferrin, 2 U/ml Heparin and 1 μg/ml DOX for 6 days.

Colony-forming unit (CFU) assay: The lineage minus (Lin⁻) cells were isolated by depletion of lineage-committed cells in bone marrow MNCs using the mouse lineage cell depletion kit (Miltenyi Biotec, San Diego, Calif.) according to the manufacturer's instructions. CFU assays were performed using ColonyGEL (Reachbio, Seattle, Wash.) with mouse complete medium according to the manufacturer's protocol. Colonies were scored 10 days after plating.

T7EI mismatch nuclease assay: Genomic DNA was isolated using PureLink Genomic DNA Mini Kit per provided protocol (Life Technologies, Carlsbad, Calif.) (Miller et al., Nat Biotechnol 25: 778-785, 2007). A genomic segment encompassing the targeted site of HBG1/2 promoter was amplified by PCR primers: HBG1/2 forward (SEQ ID NO: 270), reverse (SEQ ID NO: 271). PCR products were hybridized and treated with 2.5 Units of T7EI (NEB) for 20 minutes at 37° C. Digested PCR products were resolved by 10% TBE PAGE (Bio-Rad) and stained with ethidium bromide. 100 bp DNA Ladder (New England Biolabs) were used. Band intensity was analyzed using ImageJ software. % cleavage=(1-sqrt(parental band/(parental band+cleaved bands))×100%.

Flow cytometry: Cells were resuspended at 1×10⁶ cells/100 μL in PBS supplemented with 1% FCS and incubated with FcR blocking reagent (Miltenyi Biotech, Auburn Calif.) for ten minutes on ice. Next the staining antibody solution was added in 100 μL per 10⁶ cells and incubated on ice for 30 minutes in the dark. After incubation, cells were washed once in FACS buffer (PBS, 1% FBS). For secondary staining the staining step was repeated with a secondary staining solution. After the wash, cells were resuspended in FACS buffer and analyzed using a LSRII flow cytometer (BD Biosciences, San Jose, Calif.). Debris was excluded using a forward scatter-area and sideward scatter-area gate. Single cells were then gated using a forward scatter-height and forward scatter-width gate. Flow cytometry data were then analyzed using FlowJo (version 10.0.8, FlowJo, LLC). For flow analysis of LSK cells, cells were stained with biotin-conjugated lineage detection cocktail (Miltenyi Biotec, San Diego, Calif.) (cat #: 130-092-613) and antibodies against c-Kit (cat #:12-1171-83) and Sca-1 (cat #: 25-5981-82) as well as APC-conjugated streptavidin. Other antibodies from eBioscience (San Diego, Calif.) included anti-mouse LY-6A/E (Sca-1)-PE-Cyanine7 (clone D7), anti-mouse CD117 (c-Kit)-PE (Clone 2B8), anti-mouse CD3-APC (clone 17A2) (cat #:17-0032-82), anti-mouse CD19-PE-Cyanine7 (clone eBio1D3) (cat #: 25-0193-82), and anti-mouse Ly-66 (Gr-1)-PE, (clone R66-8C5) (cat #: 12-5931-82. Anti-mouse Ter-119-APC (clone: Ter-119) (cat #: 116211) was from Biolegend (San Diego, Calif.).

Intracellular flow cytometry detecting human γ-globin expression: The FIX & PERM™ cell permeabilization kit (Thermo Fisher Scientific) was used and the manufacture's protocol was followed. Briefly, 1×10⁶ cells were resuspended in 100 μl FACS buffer (PBS supplemented with 1% FCS), 100 μl of reagent A (fixation medium) was added and incubated for 2-3 minutes at room temperature, 1 ml pre-cooled absolute methanol was then added, mixed and incubated on ice in the dark for 10 minutes. The samples were then washed with FACS buffer and resuspended in 100 μl reagent B (permeabilization medium) and 1 μg hemoglobin γ antibody (Santa Cruz Biotechnology, cat #sc-21756 PE), incubated for 30 minutes at room temperature. After the wash, cells were resuspended in FACS buffer and analyzed.

Globin HPLC: Individual globin chain levels were quantified on a Shimadzu Prominence instrument with a SPD-10AV diode array detector and an LC-10AT binary pump (Shimadzu, Kyoto, Japan). Vydac 214TP™ C4 Reversed-Phase columns for polypeptides (214TP54 Column, C4, 300 A, 5 μm, 4.6 mm i.d.×250 mm) (Hichrom, UK) were used. A 40%-60% gradient mixture of 0.1% trifluoroacetic acid in water/acetonitrile was applied at a rate of 1 mL/min.

Measurement of vector copy number: For absolute quantification of adenoviral genome copies per cell, genomic DNA was isolated from cells using PureLink Genomic DNA Mini Kit per provided protocol (Life Technologies), and used as template for qPCR performed using the power SYBR™ green PCR master mix (Thermo Fisher Scientific). The following primer pairs were used: human γ-globin forward (SEQ ID NO: 195), and reverse (SEQ ID NO: 196); rngrnt forward (SEQ ID NO: 220), and reverse (SEQ ID NO: 221).

Real-time reverse transcription PCR: Total RNA was extracted from 5×10\hat{ }6 differentiated HUDEP-2 cells or 100 μl blood by using TRIzol™ reagent (Thermo Fisher Scientific) following the manufacture's phenol-chloroform extraction method. Quantitect reverse transcription kit (Qiagen) and power SYBR™ green PCR master mix (Thermo Fisher Scientific) were used. Real time quantitative PCR was performed on a StepOnePlus real-time PCR system (AB Applied Biosystems). The following primer pairs were used: mouse RPL10 (house-keeping) forward (SEQ ID NO: 189), and reverse (SEQ ID NO: 190); human γ-globin forward (SEQ ID NO: 191), and reverse (SEQ ID NO: 192); human β-globin forward (SEQ ID NO: 216), and reverse (SEQ ID NO: 217); mouse β-major globin forward (SEQ ID NO: 193), and reverse (SEQ ID NO: 194), mouse a globin forward (SEQ ID NO: 212), and reverse (SEQ ID NO: 213).

Cas9 Western Blot: 3×10⁶ HUDEP-2 cells were harvested at various time points after transduction, washed twice with PBS, and lysed with Laemmli buffer with 5% β-mercaptoethanol. The samples were boiled at 95° C. for 5 minutes and cleared by centrifugation at 13,000 g for 10 minutes. 10 μL of lysates was separated by SDS-PAGE using 4-15% precast protein gels (Bio-Rad). The Cas9 protein in the blots was probed by anti-Cas9-HRP (clone 7A9-3A3) (Cell Signaling Technology, Danvers, Mass.). Chemiluminescence detection on X-ray films was performed after treatment with Pierce™ ECL Plus Western Blotting Substrate (Thermo Fisher Scientific). After Cas9 detection, the blots were stripped and re-probed by an anti-β-actin antibody from Sigma-Aldrich (Clone AC-74) for internal control.

Animals: All experiments involving animals were conducted in accordance with the institutional guidelines set forth by the University of Washington. The University of Washington is an Association for the Assessment and Accreditation of Laboratory Animal Care International (AALAC)-accredited research institution and all live animal work conducted at this university is in accordance with the Office of Laboratory Animal Welfare (OLAW) Public Health Assurance (PHS) policy, USDA Animal Welfare Act and Regulations, the Guide for the Care and Use of Laboratory Animals and the University of Washington's Institutional Animal Care and Use Committee (IACUC) policies. The studies were approved by the University of Washington IACUC (Protocol No. 3108-01). C57Bl/6 based transgenic mice that contained the human CD46 genomic locus and provide CD46 expression at a level and in a pattern similar to humans (hCD46+/+ mice) were described earlier (Kemper et al., Clin Exp Immunol 124: 180-189, 2001). Transgenic mice carrying the wildtype 248 kb β-globin locus yeast artificial chromosome (β-YAC) were used (Peterson et al., Ann NY Acad Sci 850: 28-37, 1998). β-YAC mice were crossed with human CD46+/+ mice to obtain β-YAC^{+/-}/CD46^{+/+} mice for in vivo HSPC transduction studies. The following primers were used for genotyping of mice: CD46 forward (SEQ ID NO: 233), and reverse (SEQ ID NO: 234); β-YAC ( [symbol] -globin promoter) forward (SEQ ID NO: 242), and reverse (SEQ ID NO: 243).

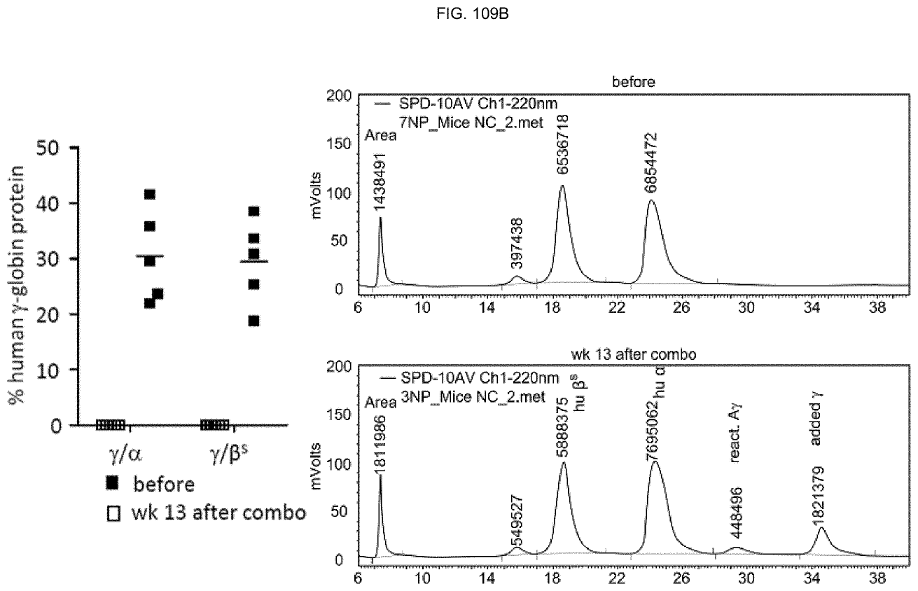

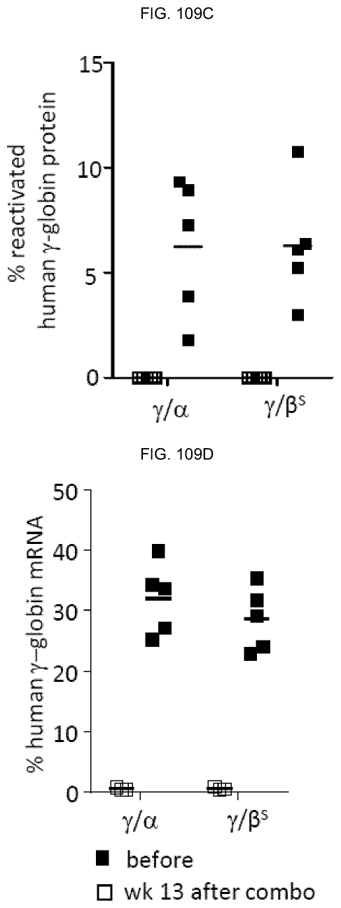

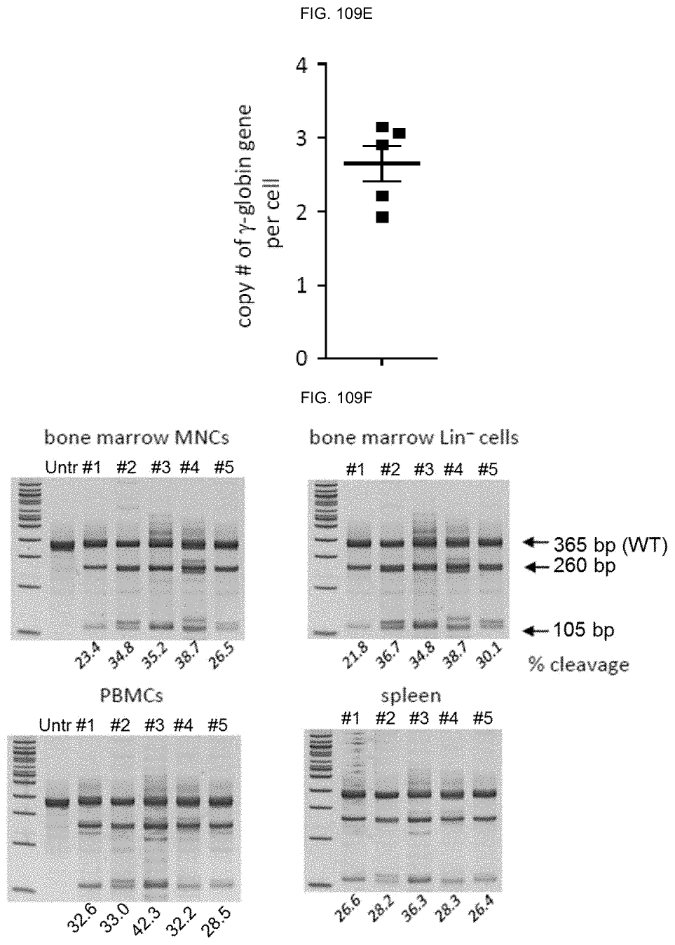

Sickle cell disease mouse model: A Townes male mouse (Hbb^{tm2(HBG1,HBB*)Tow} or hα/hα::β^{S}/β^{S}) was purchased from the Jackson Laboratory (JAX stock #013071) and bred with human CD46 transgenic female mice. As shown in FIG. 109A, after three rounds of breeding, mice homozygous for CD46, HbS and HBA were obtained and used for experiments. The following primers were used for genotyping: HBB primers (SEQ ID NOs: 246, 251, and 70), and HBA primers (SEQ ID NOs: 272-274); and CD46 primers as shown above (SEQ ID NOs: 233 and 234). The PCR results were interpreted according to the provided protocols by the vendor.

HSPC mobilization and in vivo transduction: HSPCs were mobilized in mice by s.c. injections of human recombinant G-CSF (5 μg/mouse/day, 4 days) followed by an s.c. injection of AMD3100 (5 mg/kg) on day 5. In addition, animals received Dexamethasone (10 mg/kg) i.p. 16 h and 2 h before virus injection. Thirty and 60 minutes after AMD3100, animals were intravenously injected with virus vectors through the retro-orbital plexus with a dose of 4×10¹⁰ viral particles (vp) per injection.

In vivo selection: Selection was started at one week (Townes model) or four weeks (β-YAC model) after transduction. Mice were injected with O⁶-BG (15 mg/kg, IP) two times, 30 minutes apart. One hour after the second injection of O⁶-BG, mice were injected (IP) with 5 mg/kg Carmustine (BCNU). At two and four weeks after the first round of selection, two more rounds were performed with BCNU doses at 7.5 and 10 mg/kg, respectively.

Immunosuppression: Mycophenolate mofetil (CellCept Intravenous) was from Genentech (Hillsboro, Oreg.). Rapamycin (Rapamune/Sirolimus) and methylprednisolone were from Pfizer (New York, N.Y.). Daily intraperitoneal injection of a mycophenolate mofetil (20 mg/kg/day), rapamycin (0.2 mg/kg/day), methylprednisolone (20 mg/kg/day) was performed.

Secondary bone marrow transplantation: Recipients were female C57BL/6 mice, 6-8 weeks old from the Jackson Laboratory. On the day of transplantation, recipient mice were irradiated with 1000 Rad. Bone marrow cells from in vivo transduced CD46tg mice were isolated aseptically and lineage-depleted cells were isolated using MACS as described above. Six hours after irradiation cells were injected intravenously at 1×10⁶ cells per mouse. The secondary recipients were kept for 16 weeks after transplantation for terminal point analyses. All secondary recipients received immunosuppression starting at week 4.

Tissue analysis: Spleen and liver tissue sections of 2.5 μm thickness were fixed in 4% formaldehyde for at least 24 hours, dehydrated and embedded in paraffin. Staining with hematoxylin-eosin was used for histological evaluation of extramedullary hemopoiesis. Hemosiderin was detected in tissue sections by Perl's Prussian blue staining. Briefly, the tissue sections were treated with a mixture of equal volumes (2%) of potassium ferrocyanide and hydrochloric acid in distilled water and then counterstained with neutral red. The spleen size was assessed as the ratio of spleen weight (mg)/body weight (g).

Blood analysis: Blood samples were collected into EDTA-coated tubes and analysis was performed on a HemaVet 950FS (Drew Scientific, Waterbury, Conn.). Peripheral blood smears were stained with Giemsa/May-Grunwald (Merck, Darmstadt, Germany) for 5 and 15 minutes, respectively. Reticulocytes were stained with Brilliant cresyl blue. The investigators who counted the reticulocytes on blood smears have been blinded to the sample group allocation. Only animal numbers appeared on the slides. (5 slides per animal, 5 random 1 cm² sections)

Statistical analyses: For comparisons of multiple groups, one-way and two-way analysis of variance (ANOVA) with Bonferroni post-testing for multiple comparisons was employed. Statistical analysis was performed using Graph Pad Prism version 6.01 (GraphPad Software Inc., La Jolla, Calif.).

Results and Discussion

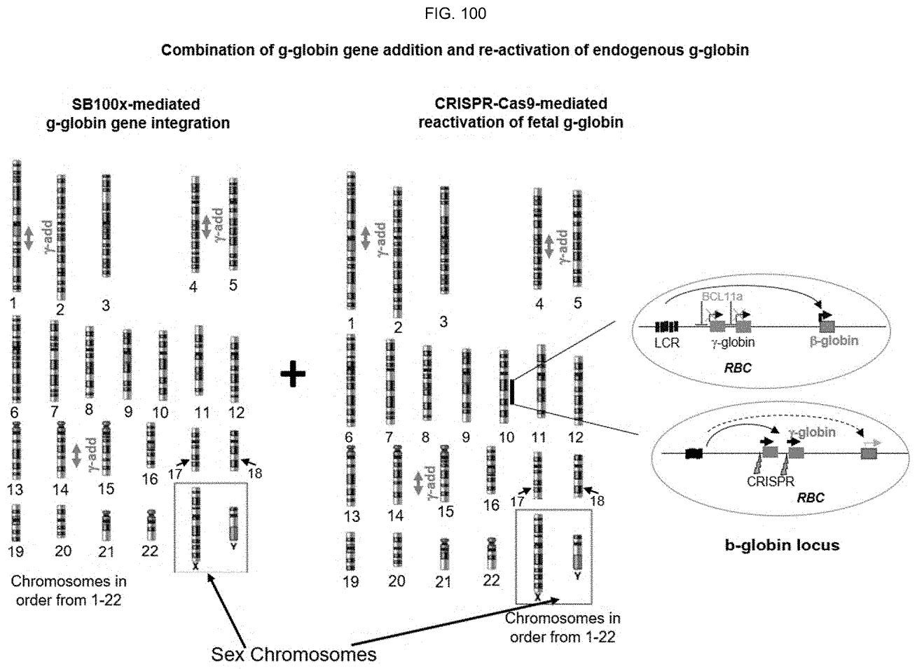

HDAd-combo vector for [symbol] -globin gene addition and self-inactivating CRISPR/Cas9 for γ-globin re-activation: The 30 kb insert capacity of HDAd5/35++ vectors was capitalized on to incorporate two therapeutic cassettes into one vector (FIG. 100, upper panel, “HDAd-combo”): i) a cassette for [symbol] -globin gene addition by SB100x consisting of a HS1-HS4 mini-LCR in combination with a β-globin promoter to drive human [symbol] -globin expression (Wang et al., J Clin Invest 129: 598-615, 2019). This cassette is linked to the gene for a mutant O⁶-methylguanine-DNA methyltransferase (mgmt^{P140K}) under control of the ubiquitously active PGK promoter to allow for selection of stably transduced cells by low-dose O⁶BG/BCNU treatment (Neff et al., J Clin Invest 112: 1581-1588, 2003; Wang et al., Mol Ther Methods C.lin Dev 8: 52-64, 2018). The γ-globin/mgmt^{P140K} transposon cassette is flanked by frt sites and IRs, a CRISPR/Cas9 expression cassette that was placed outside the IR/frt flanked transposon. This module consists of a U6-promoter driven sgRNA targeting the BCL11A binding site within the HBG1/2 promoters and a SpCas9 under the control of the EF1α promoter. Co-infection of HDAd combo and HDAd-SB and expression of SB100x and Flpe recombinase will mediate integration of the IR-flanked γ-globin/mgmt^{P140K} cassette and simultaneously destroy the vector and stop CRISPR/Cas9 expression (FIG. 101). This shortened expression of CRISPR/Cas9 should increase the survival of genome-edited cells and the percentage of long-term repopulating cells. For comparison, HDAd5/35++ vectors were included in the study that contained the two different modules separately, HDAd-CRISPR (“cut”) and HDAd-SB-addition (“add”) (FIG. 100, middle panels-“HDAd-cut” and “HDAd-SB-add”).

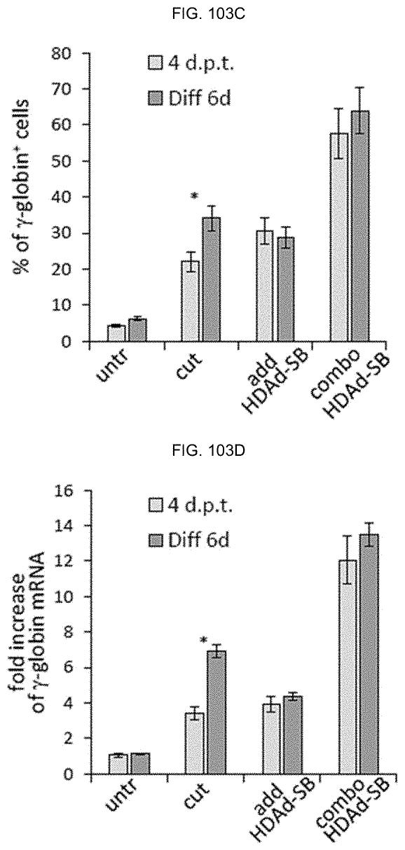

Vector validation in HUDEP-2 cells: The hypothesis was first tested in Human Umbilical cord blood-Derived Erythroid Progenitor (HUDEP-2) cells (Kurita et al., PLoS One 8: e59890, 2013), an immortalized human hematopoietic stem and progenitor cell-derived erythroid precursor cell line that expresses BCL11A and predominantly β-globin and, only low levels of γ-globin. HUDEP-2 cells have been widely used for [symbol] -globin re-activation studies (Canver et al., Nature 527: 192-197, 2015). Four days after infection of HUDEP-2 cells with HDAd-combo+/−HDAd-SB at an MOI that transduces the vast majority of cells, cells were further expanded for 8 days in erythroid differentiation medium as described earlier (Li et al., Mol Ther 27: 2195-2212, 2019). Cas9 Western blot signals sharply declined once cells were subjected to differentiation/expansion, most likely due to the loss of episomal HDAd-combo vector copies (FIG. 103A). A schematic for controlled Cas9 expression using HDAd-combo vectors is shown in FIG. 102. Cas9 was however detectable for the period of the study 12 days. Co-infection with HDAd-SB reduced Cas9 expression 35% (Diff d3) to 50% (Diff d8) (FIG. 103B), indicating an effect of the self-inactivation mechanism described in FIG. 101. Analysis of [symbol] -globin marking by flow cytometry (FIG. 103C) suggested an additive effect of the γ-globin gene addition and reactivation modules.

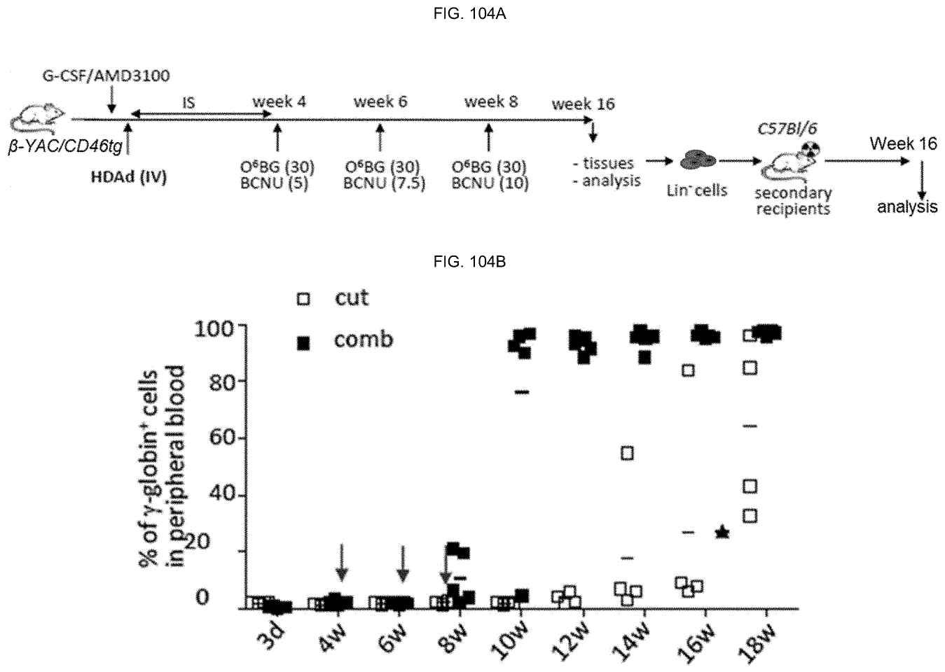

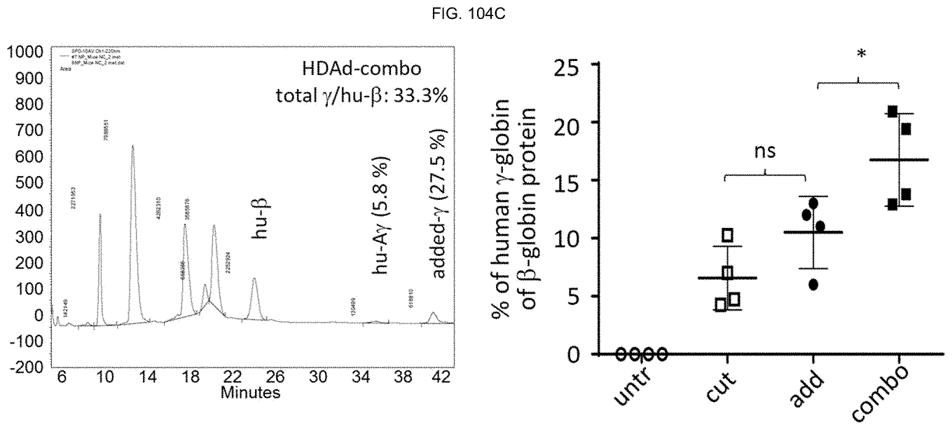

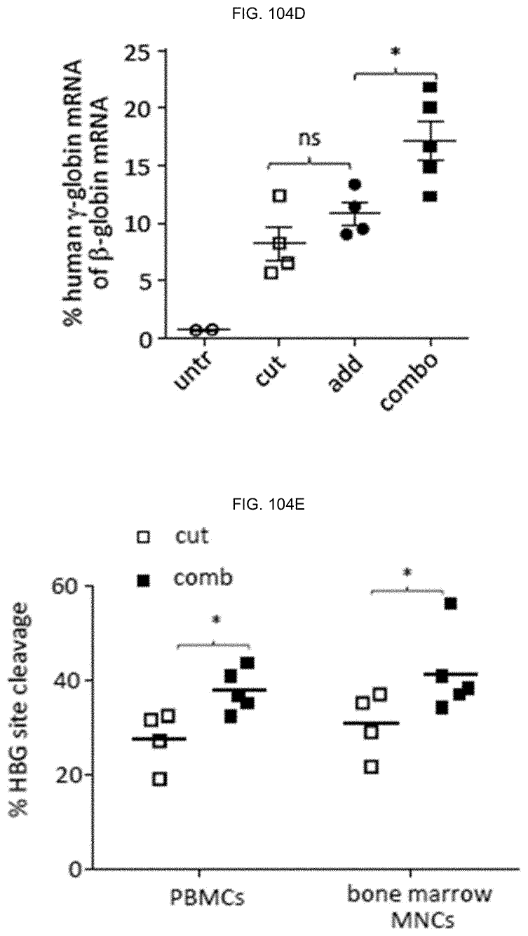

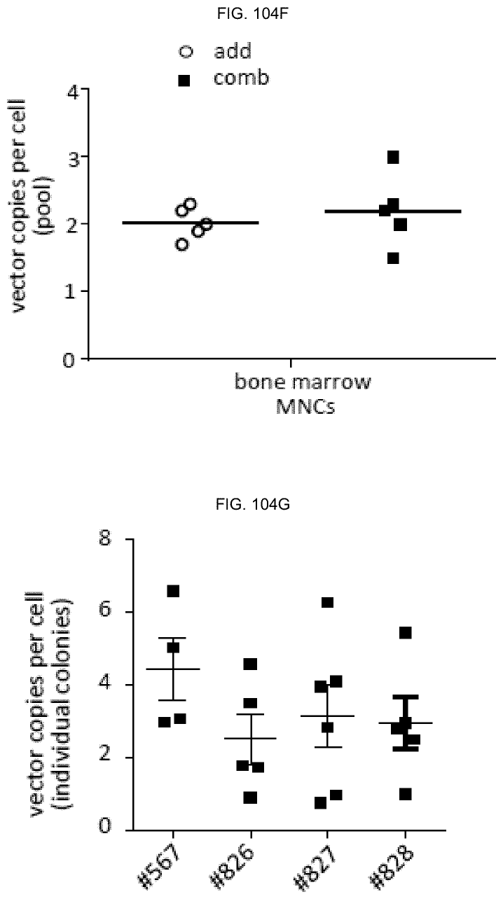

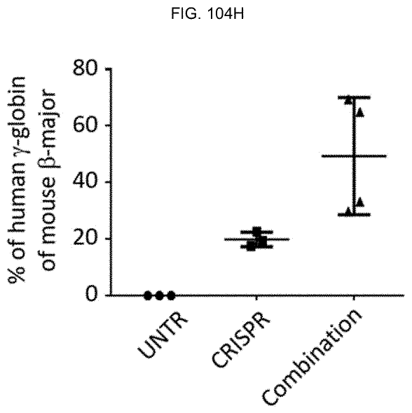

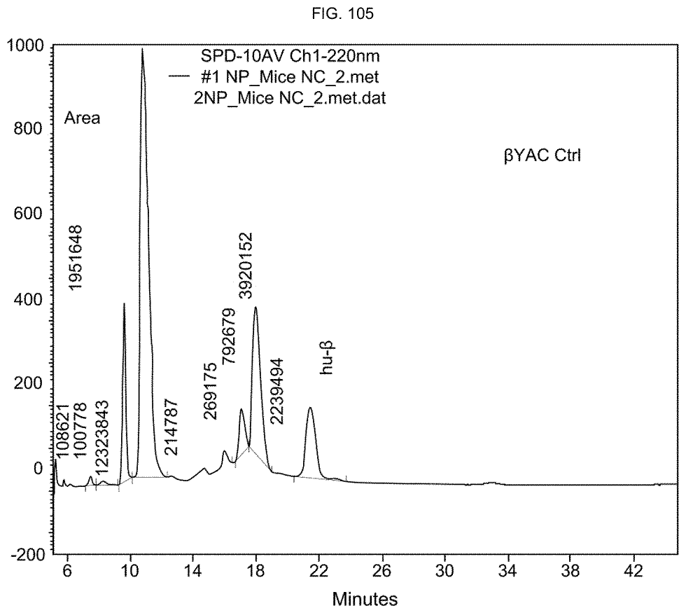

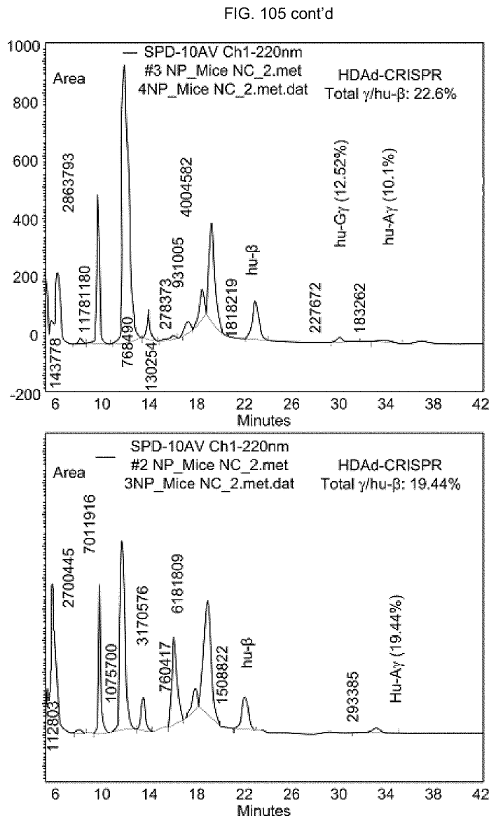

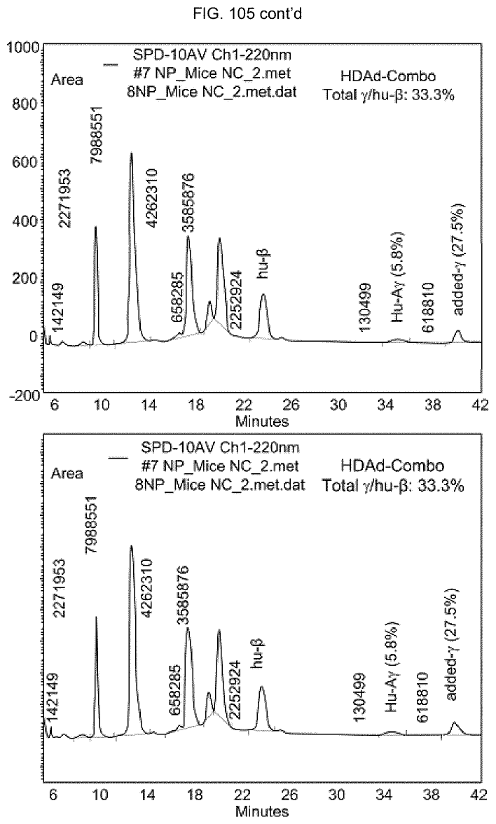

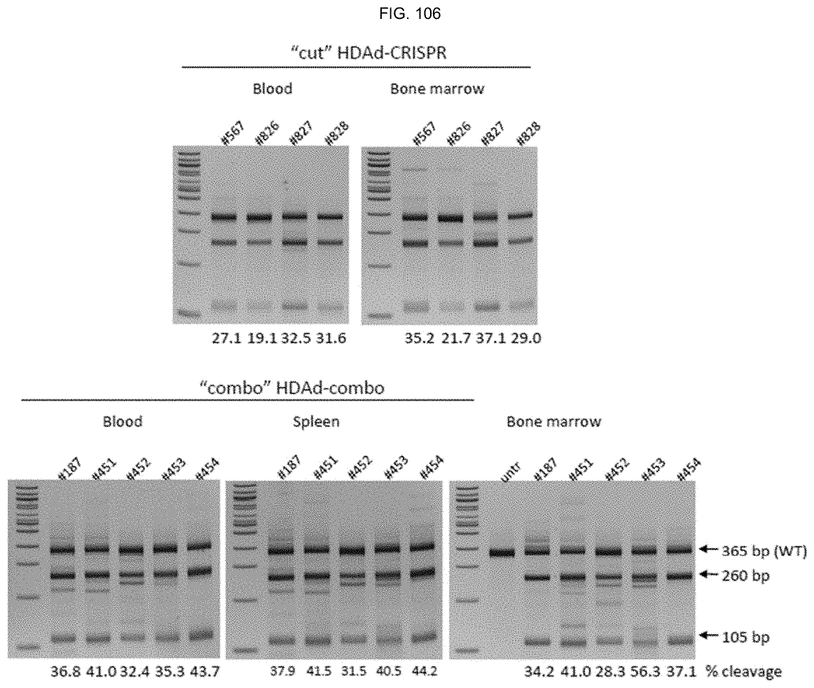

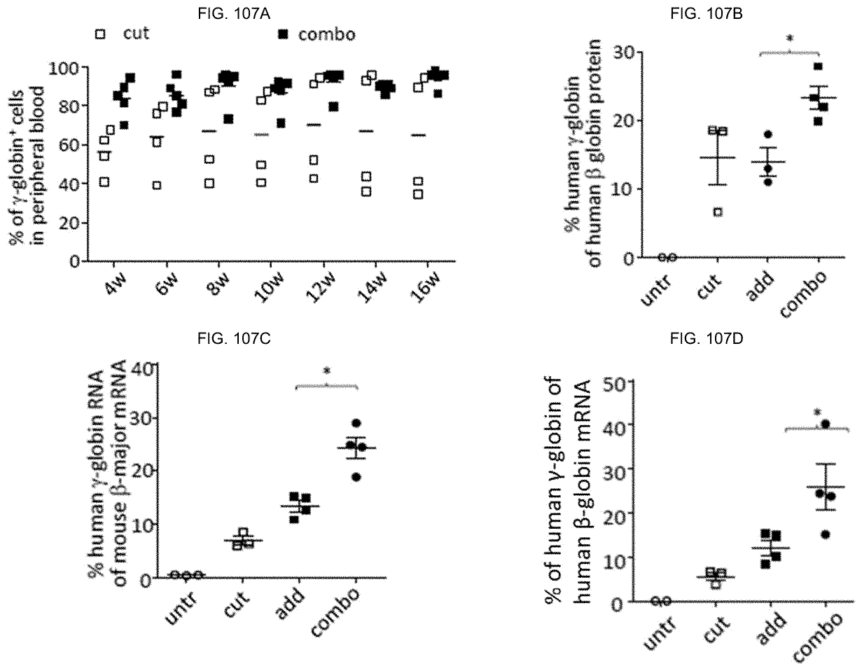

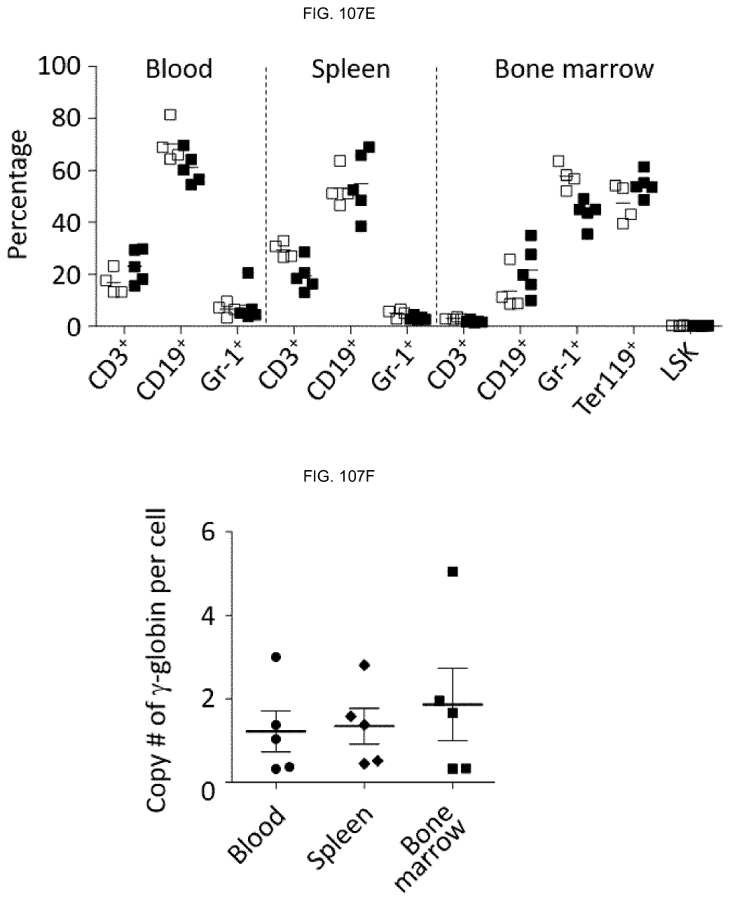

In vivo HSPC transduction in CD46/β-YAC mice. It has been previously demonstrated human γ-globin reactivation CD46/β-YAC mice after in vivo HSPC transduction with and HDAd5/35++ vector targeting the HBG1/2 promoter (Li et al., Blood 131: 2915-2928, 2018). Here, a similar protocol was followed to evaluate the new HDAd-combo vector. CD46/β-YAC mice were mobilized with G-CSF/AMD3100, intravenously injected with the “cut”. “add” and “combo” vectors and, four weeks later, subjected to three rounds of in vivo selection (FIG. 104A). The percentage of γ-globin-positive RBCs increased with each round of in vivo selection reaching >95% for the “combo” vector 2 weeks after the last round of O⁶BG/BCNU injection (FIG. 104B). Reactivation with the “cut” vector was less efficient (60%) and more variable between animals. At week 18, RBC lysates were analyzed by HPLC for globin chains. The chromatogram shows distinct peaks for human β-globin, reactivated human G [symbol] /A [symbol] (HBG1/2) and the added 76-Ile G [symbol] variant (Li et al., Mol Ther Methods Clin Dev 9: 142-152, 2018) (FIG. 104C left panel, FIG. 105). Notably, the simultaneous reactivation of G [symbol] and A [symbol] was seen only in a small fraction of mice treated with the “cut” vector (FIG. 105). The majority of “cut” and “combo” vector-treated mice displayed only reactivated A [symbol] , most likely due to the deletion of the HBG2 gene as a result of simultaneous cleavage of the CRISPR/Cas9 in both the HBG1 and HBG2 promoters (Li et al., Blood 131: 2915-2928, 2018). FIG. 104C (right panel) shows γ-globin protein levels relative to human β-globin. On average, 7%, 11% and 17% γ-globin protein were detected for the “cut”, “add”, and “combo” vector, respectively. A similar pattern was seen on the mRNA level (FIG. 104D). While the difference between the “cut” and “add” vectors were not significant, the level of γ-globin for the “combo” vector was significantly higher. The percentage of CRISPR/Cas9-mediated cleavage of the HBG promoter target site measured at week 18 in PBMCs and bone marrow MNCs was significantly higher for the “combo” vector compared to the “cut” vector (FIG. 104E, FIG. 106). This is most likely due to the mechanism leading to reduced CRISPR/Cas9 expression, and, potentially, better survival of CRISPR-edited HSPCs that were then expanded by in vivo selection. The vector copy number in bone marrow MNCs was comparable for the “add” and “combo” vector, excluding that the increased γ-globin levels for the “combo” vector were due to better transduction and vector integration (FIG. 104F). When analyzed in individual progenitor colonies in different mice, the VCN ranged from 1 to 6 copies per cell (FIG. 104G). To demonstrate that [symbol] -globin gene addition and CRISPR cleavage-mediated [symbol] -globin reactivation occurred in long-term repopulating HSCs, bone marrow Lin⁻ cells were transplanted, harvested at week 18 after in vivo HSPC transduction of β-YAC/CD46 mice with the “cut” and “combo” vector, into lethally irradiated C57Bl/6 mice. The ability of transplanted cells to drive the multi-lineage reconstitution in secondary recipients was assessed over a period of 16 weeks. Engraftment rates based on CD46 expression in PBMCs were 95% and remained stable. γ-globin marking of RBCs measured by flow cytometry was also stable and in the range of 70% and 95% at week 16 for the “cut” and “combo” vector, respectively (FIG. 107A). γ-globin expression levels (relative to mouse β-major) measured by HPLC (FIG. 107B) or qRT-PCR (FIG. 107C) were comparable to primary mice. FIG. 107B shows the level of γ-globin protein relative to human β-globin at week 16 after transplantation. FIGS. 107C and 107D show the level of γ-globin protein relative to mouse βₘₐⱼₒᵣ-globin and human β-globin.

No effect from the genetic manipulation of HSPCs or γ-globin expression from erythroid cells on the cellular composition of the blood, spleen, and bone marrow was observed. FIG. 107E Lineage-positive cell composition in MNCs of blood, spleen, and bone marrow at week 16 after transduction with the “combo” vector (solid symbols) compared to untransduced control mice (unfilled symbols). FIG. 107F shows number of integrated copies of transposon per cell in blood, spleen, and bone marrow.

In vivo HSPC transduction studies in SCD (Townes) mice. In this model, the murine α-globin genes were replaced with the human α-globin and the murine adult β-globin genes were replaced with human sickle β^{S} and fetal γ-globin genes linked together. The β-globin gene (HBG1) contains 1400 bp of 5′ flanking sequence, which contains the BCL11A target site cleaved by the CRISPR/Cas9. This should lead to reactivation of the β-globin gene. The genome of the Townes model is better characterized than that of another SCD mouse model, the Berkeley model (Hba^{0/0} Hbb^{0/0} Tg(Hu-miniLCRα1^{G}γ^{A}γδβ^{S}), which appears to have more than two copies of the human globin transgenes (Paszty et al., Science 278: 876-878, 1997).

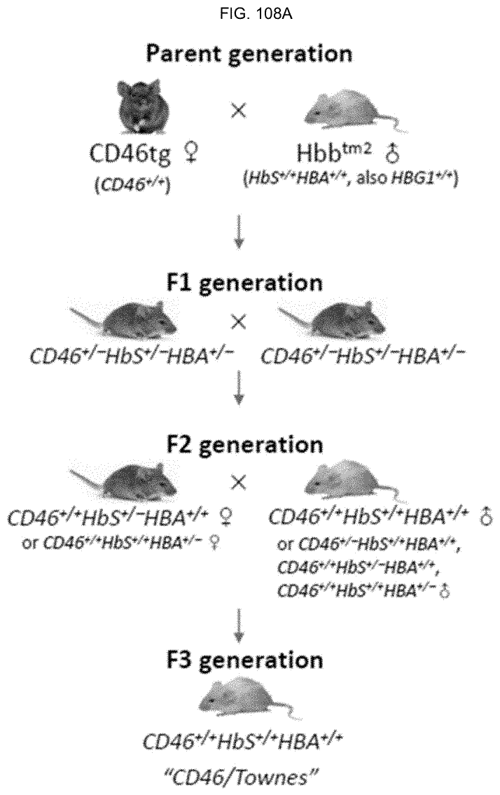

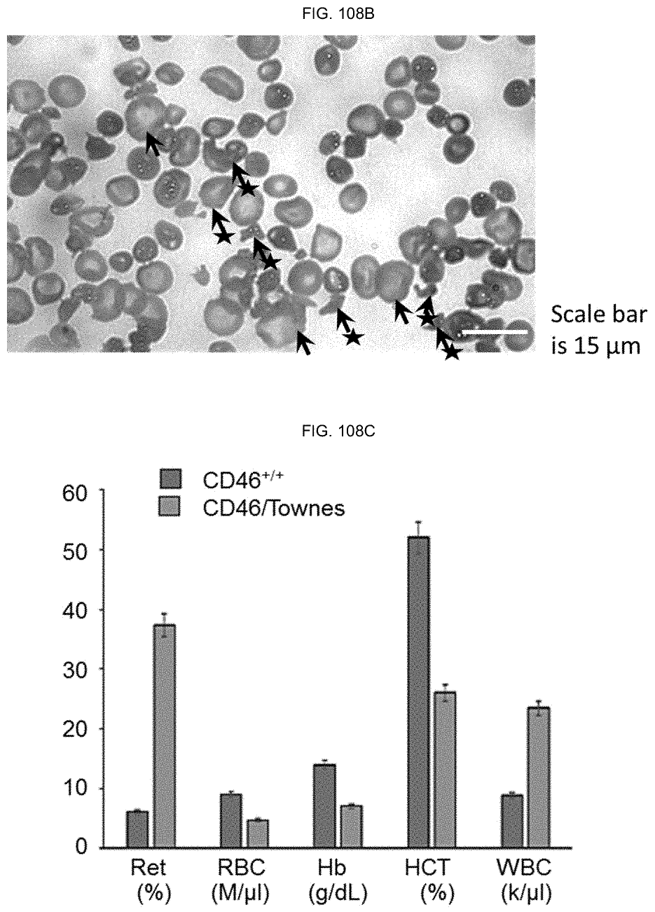

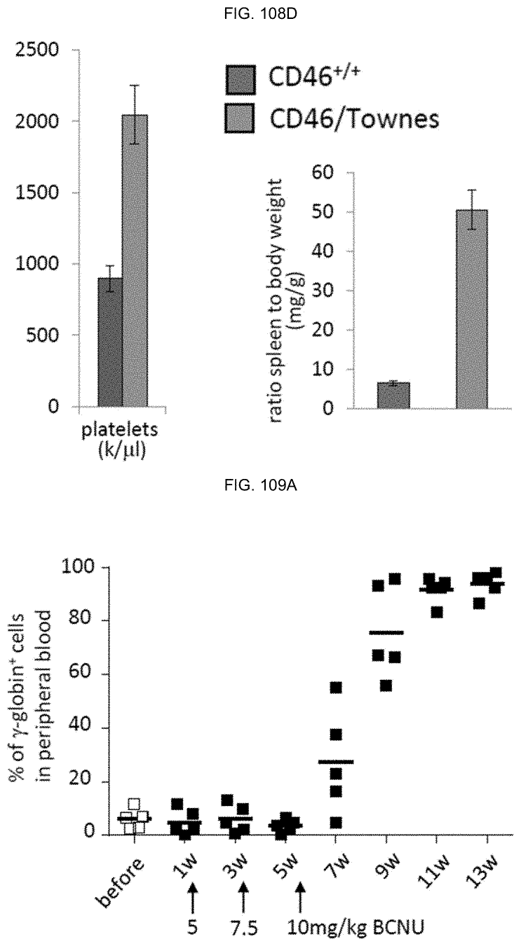

To make the Townes model suitable for HDAd5/35++ HSPC gene therapy, Townes mice were bred with human CD46 transgenic mice. After three rounds of backcrossing, mice homozygous for human CD46 and the two human (α, β^{S}/ [symbol] ) globin genes were used for experiments (FIG. 108A). Triple homozygous CD46/Townes mice displayed sickle-like erythrocytes (FIG. 108B), severe anemia, 40% reticulocytes in the peripheral blood as well as leukocytosis and thrombocytosis (FIG. 108C). The latter indicates that the disturbance of hematopoiesis extend beyond the erythroid lineage. Another characteristic feature was splenomegaly as a result of extramedullary hematopoiesis (FIG. 108D).

CD46/Townes mice were mobilized with GCSF/AMD3100 and intravenously injected with the HDAd-combo+HDAd-SB vectors. In vivo selection with O⁶BG/BCNU was started one week after transduction and repeated at weeks 4 and 6 with increasing BCNU doses (5→7.5→10 mg/kg). At baseline, on average, 5% of RBCs were γ-globin positive with a low MFI, indicating an incomplete repression of fetal globin in CD46/Townes mice. After three rounds of in vivo selection, the percentage of -globin-positive RBCs increased and reached >95% by the end of the study (week 13 after in vivo transduction) (FIG. 109A). HPLC analysis of RBC lysates showed γ-globin levels that were 30% of human α-globin or β^{S}-globin (FIG. 109B left panel). Peaks for added γ-globin and re-activated Ay were clearly visible (FIG. 109B right panel). As seen in the CD46/β-YAC model, reactivated [symbol] -globin contributed less than the added [symbol] -globin to total [symbol] -globin levels (FIG. 109C). The low level of baseline [symbol] -globin detected by flow cytometry was below the detection limit of the HPLC. Analysis of globin mRNA in RBCs mirrored the values seen at the protein level be HPLC (FIG. 109D). The γ-globin level after HDAd-combo in vivo HSC gene therapy was higher in the SCD CD46/Townes model than in “healthy” CD46/β-YAC mice.

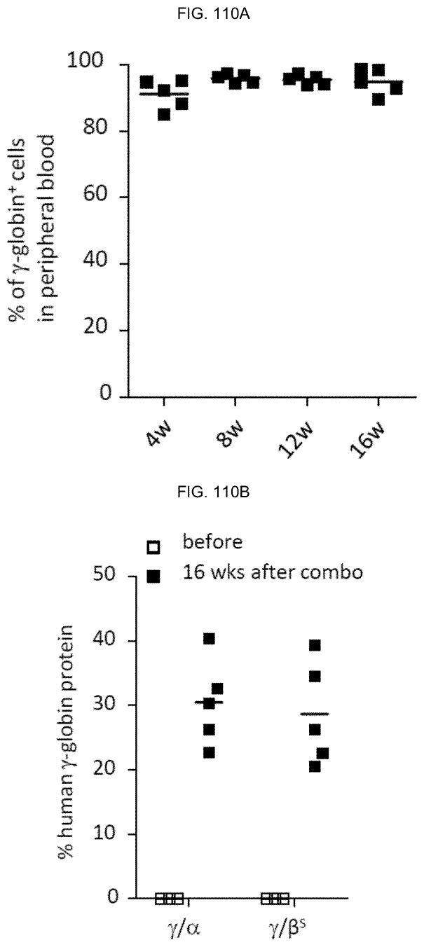

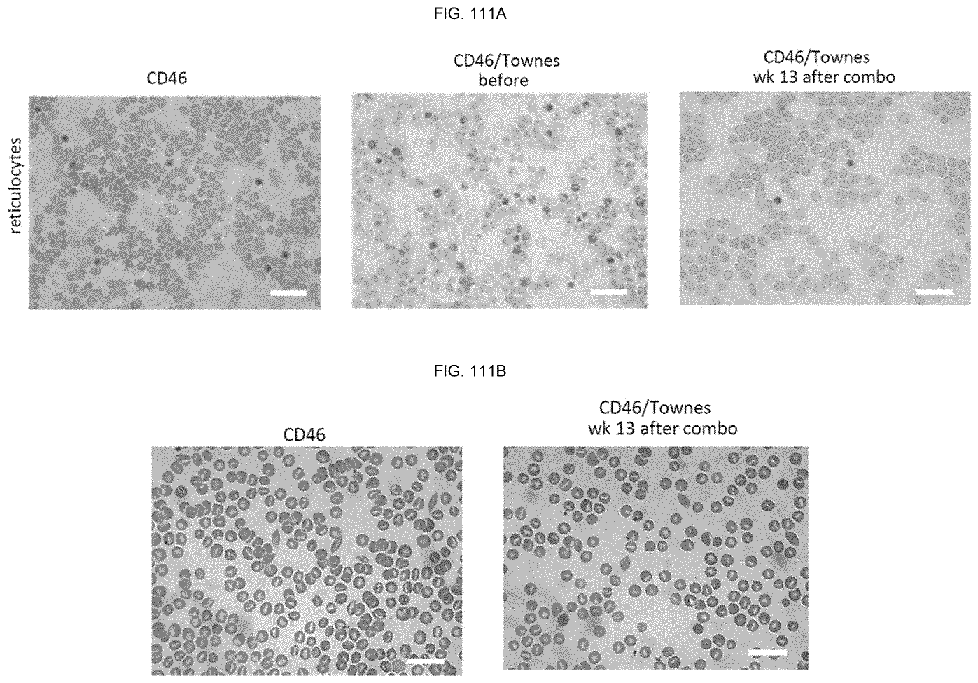

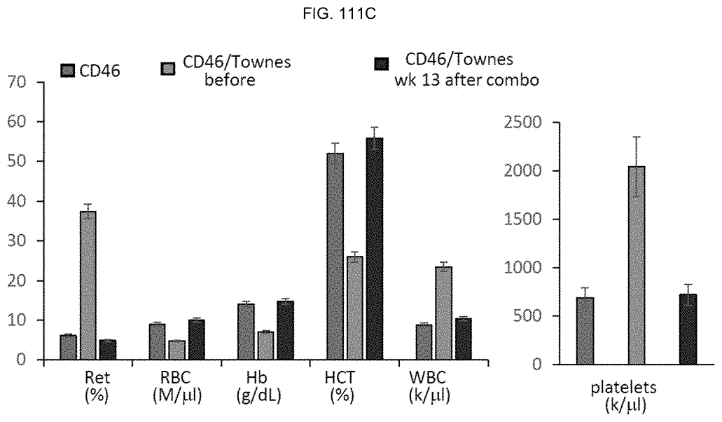

Both intended genomic modification were detected in bone marrow samples from week 13. On average 2.5 integrated γ-globin genes were found per cell (FIG. 109E). Target site cleavage efficiency measured by T7E1 assay was comparable, in the range of 25-30% in total bone marrow MNCs, Lin⁻ cells, PBMCs, and splenocytes (FIG. 109F). To show stable genetic modification of CD46/Townes HSPCs, Lin⁻ cells harvested at week 13 after in vivo transduction were transplanted into secondary lethally irradiated C57Bl/6 recipients. γ-globin marking in RBCs was stable over 16 weeks (FIG. 110A) at a level of 30% of adult human globin (FIG. 110B).

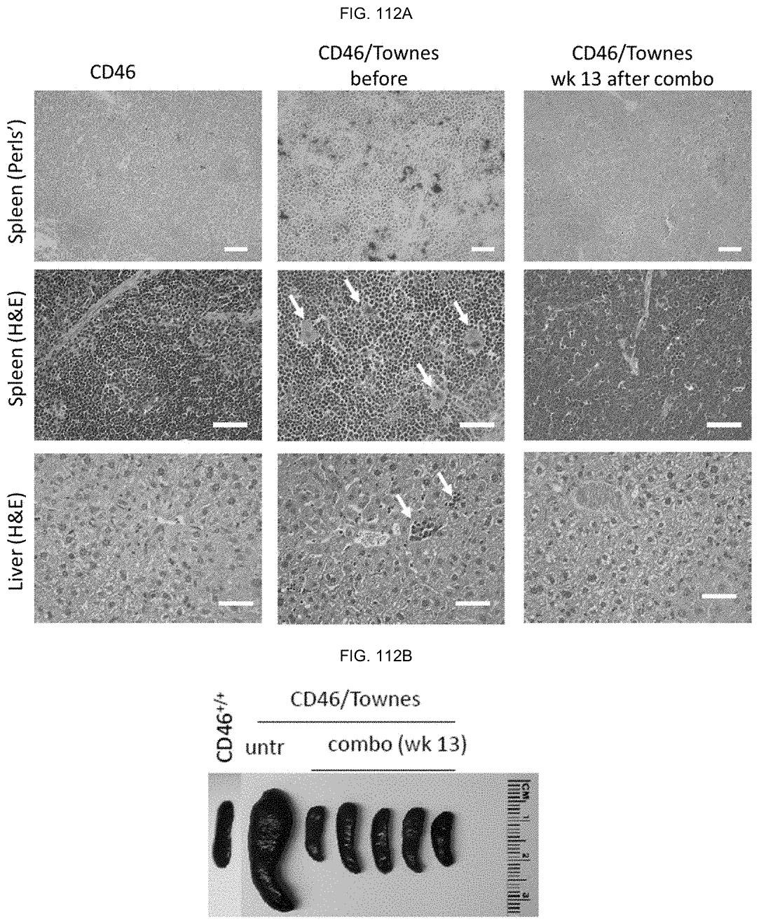

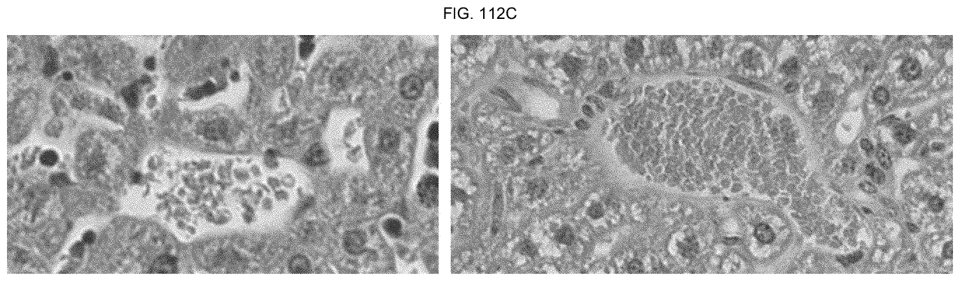

Phenotypic correction of SCD in the mouse model: At week 13 after in vivo HSPC transduction with the combo vector, phenotypic features of Sickle Cell Disease were analyzed in CD46/Townes mice. The average percentage of reticulocytes counted on peripheral blood smears was 5, 39, and 5% for parental (“healthy”) CD46 transgenic mice, CD46/Townes mice before treatment, and CD46/Townes mice at week 13 after treatment, respectively (FIGS. 111A and 111C). In treated mice, the red cell morphology in blood smears of CD46/Townes mice characterized by hypochromia, widely varying sizes/shapes (sickle cells) and cell fragmentation (see FIG. 108B), returned to the normocytic red cell appearance seen in CD46 mice (FIG. 111B). Hematological parameters including RBC, WBC, and platelet counts as well as erythroid features (e.g. hemoglobin and hematocrit) were similar in CD46 and treated CD46/Townes mice (FIG. 111C). Likewise, histological analyses of liver and spleen from treated CD46/Townes mice showed normalization, including absence of parenchymal iron deposition and extramedullary hemopoiesis (FIG. 112A). Spleen size, a measurable characteristic of compensatory hemopoiesis, in treated CD46/Townes mice was comparable to paternal CD46 mice (FIG. 112B).

Overall, these data indicate a complete cure of Sickle Cell Disease in CD46/Townes mice. It is postulated that this is directly related to the high γ-globin levels (>20%) achieved by a combination of SB100x transposase-mediated γ-globin gene addition (main contribution) and CRISPR/Cas9-triggered reactivation of endogenous γ-globin. Furthermore, these results demonstrate a reduction of Cas9 expression by Flpe/SB100x mediated excision of the CRISPR/Cas9 expression cassette from the HDAd-combo genome, leading to increased safety and percentage of CRISPR-edited HSPCS. Further improvements of this system could include approaches to decrease the amounts of β^{S} in RBCs, for example by the inclusion of Prime Editors that correct the SCD mutation into the HDAd-combo vector.

### Example 4. Production of Ad35 Vectors

This example describes the production of Ad35 vectors and demonstration of efficacy for transduction of CD34+ cells. Three exemplary Ad35 vectors were produced, with different structures (including different LoxP placement).

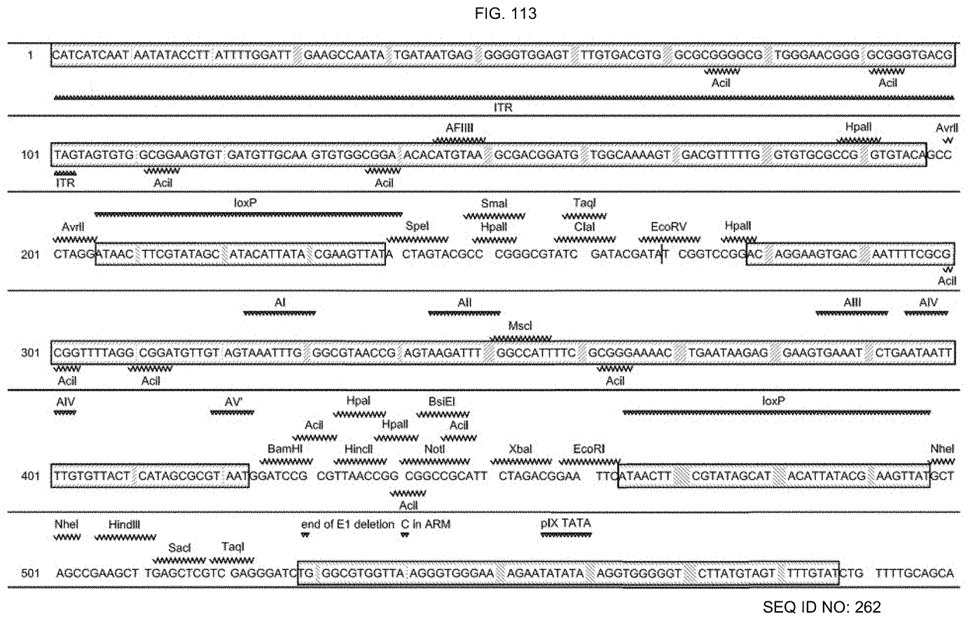

The left end of a representative Ad5/35 helper virus genome is shown in FIG. 113. The sequences shaded in dark grey correspond to the native Ad5 sequence, i.e., the unshaded or light grey highlighted sequences were artificially introduced. The sequences highlighted in light grey are two copies of the (tandemly repeated) loxP sequences. In the presence of “cre recombinase” protein, the nucleotide sequence between the two loxP sequences are deleted (leaving behind one copy of loxP). Because the Ad5 sequence between the loxP sites is essential for packaging the adenoviral DNA into capsids (in the nucleus of the producer cell), this deletion renders the helper adenovirus genome DNA deficient for packaging. Consequently, the efficiency of the deletion process has a direct influence of the level of packaged helper genomic DNA (the undesired helper virus “contamination”). In view of the above, in order to translate the same scheme to adenovirus serotypes other than Ad5, it is desirable to achieve the following: 1. Identify the sequences that are essential for packaging, so that they can be flanked by loxP sequence insertions and deleted in the presence of cre recombinase. Identification of these sequences is not straightforward if there is little similarity in sequences. 2. Determine where in the native DNA sequence the insertion of loxP sequence would have the least effect for the propagation and packaging of helper virus (in the absence of cre recombinase). 3. Determine the spacing between the loxP sequences to allow for efficient deletion of packaging sequences and keeping helper virus packaging to a minimum during the production of helper-dependent adenovirus (i.e., in a cre recombinase-expressing cell line such as the 116 cell line).

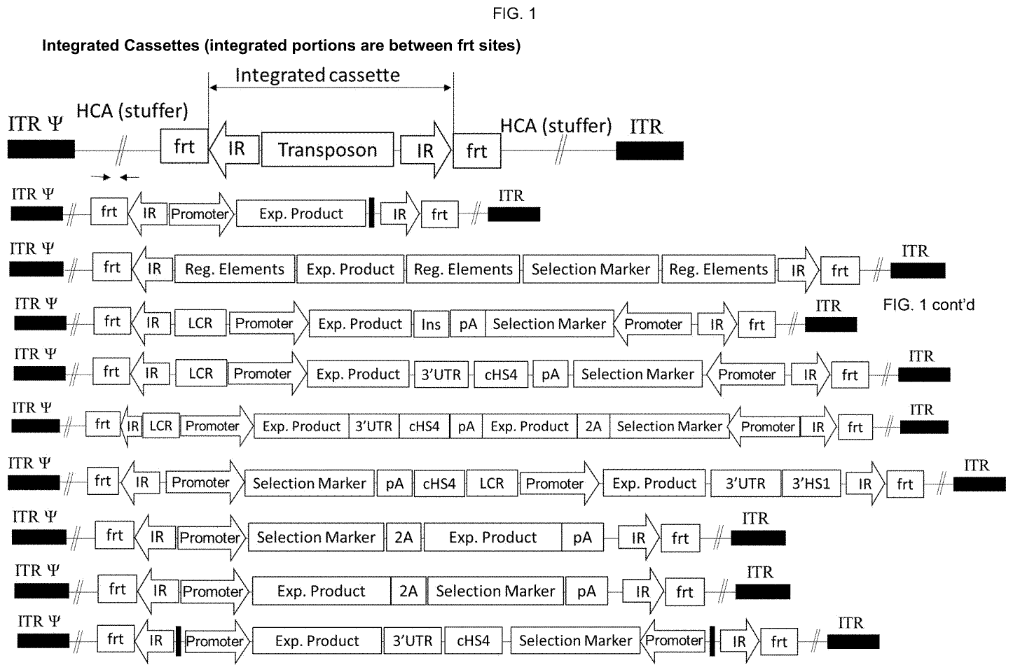

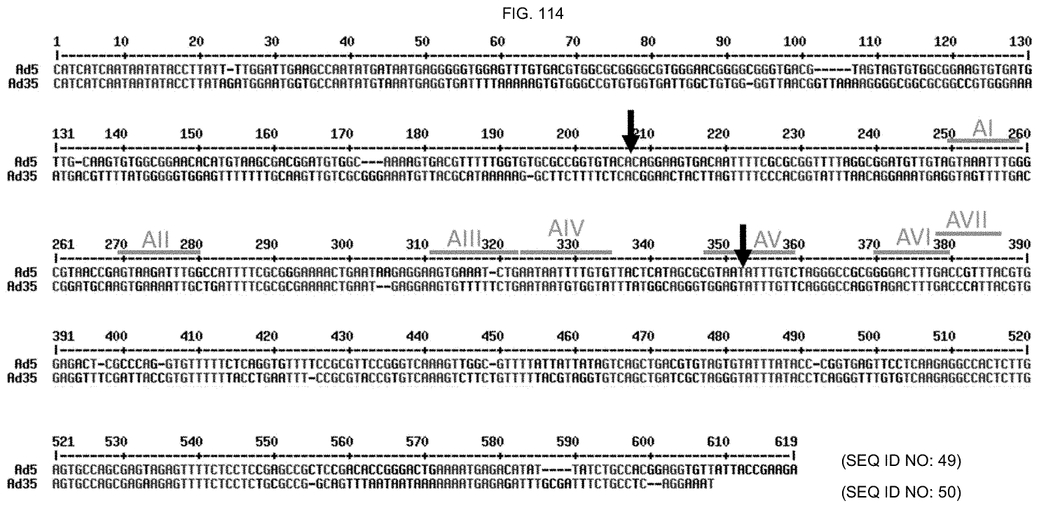

FIG. 114 shows an alignment of representative Ad5 and Ad35 packaging signals (SEQ ID NOs: 49 and 50). The alignment of the left end sequences of Ad5 with Ad35 help in identifying packaging signals. Motifs in the Ad5 sequence that are important for packaging (A1 through AV) are indicated with lines (see also FIG. 1B of Schmid et al., J Virol., 71(5):3375-4, 1997). The location of exemplary loxP insertion sites are indicated by black arrows. These insertions flank AI to AIV and disrupt AV. The additional packaging signal AVI and AVII, as indicated in Schmid et al., have been deleted in the Ad5 helper virus as part of the E1 deletion of this vector.

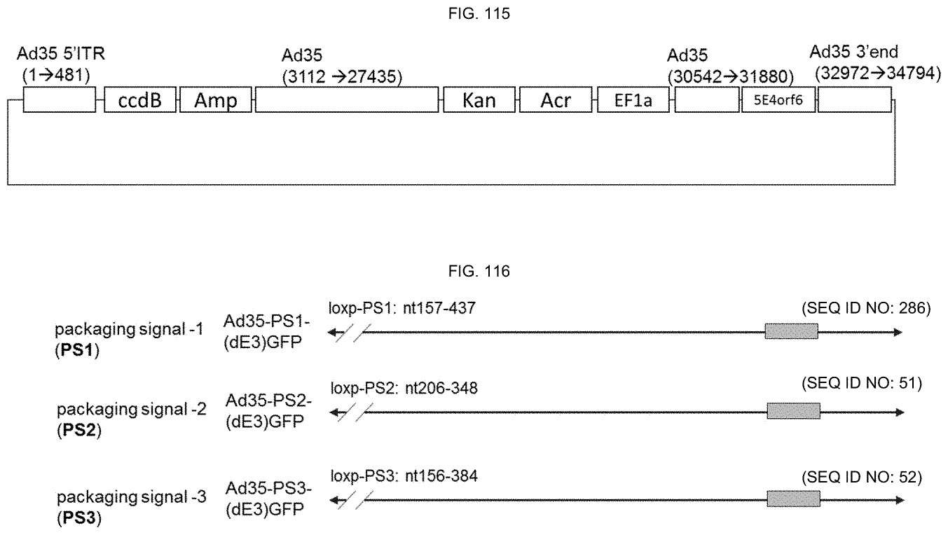

FIG. 115 is a schematic illustration of the Ad35 vector pAd35GLN-5E4. This is a first-generation (E1/E3-deleted) Ad35 vector derived from a vectorized Ad35 genome (Holden strain from the ATCC) using a recombineering technique (PMID: 28538186). This vector plasmid was then used to insert loxP sites.

The packaging site (PS)1 LoxP insertion sites are after nucleotide 178 and 344; this Ad35 vector is exemplified in SEQ ID NO: 286. This LoxP placement is expected to remove AI to AIV. The rest of the packaging signal including AVI and AVII (after 344) has been deleted (as part of the E1 deletion at positions 345 to 3113). The PS2 LoxP insertion sites are after nucleotide 178 and 481; this Ad35 vector is exemplified in SEQ ID NO: 51. Additionally, nucleotides 179 to 365 have been deleted, so AI through AV are not present. The remaining packaging motifs AVI and AVII are removable by cre recombinase during HDAd production. The E1 deletion is from 482 to 3113. The PS3 LoxP insertion sites are after nucleotide 154 and 481; this Ad35 vector is exemplified in SEQ ID NO: 52. The packaging signal structure of these three vectors is provided in FIG. 116.

Three engineered vectors could be rescued. The percentage of viral genomes with rearranged loxP sites was 50, 20, and 60% for PS1, PS2, and PS3, respectively. Rearrangements occur when the lox P sites critically affected viral replication and gene expression.

This HDAd35 platform compared to current HDAd5/35 platform is illustrated in FIG. 117. Both vectors contain a CMV-GFP cassette. The Ad35 vector does not contain immunogenic Ad5 capsid protein. These two vectors showed comparable transduction efficiency of CD34+ cells in vitro. Bridging study shows comparable transduction efficiency of CD34+ cells in vitro. Human HSCs, peripheral CD34+ cells from G-CSF mobilized donors were transduced with HDAd35 (produced with Ad35 helper P-2) or a chimeric vector containing the Ad5 capsid with fiber from Ad35, at MOIs 500, 1000, 2000 vp/cell. The percentage of GFP-positive cells was measured 48 hours after adding the virus in three independent experiments.

The PS2 helper vector was remade (as illustrated in FIG. 118) for use in monkey studies. The following are actions were taken to make this version: deletion of E1 region, a mutant packaging signal flanked by Loxp, mutant packaging sequence, deletion of E3 region (27435430540), replace with Ad5E4orf6, insertion of stuffer DNA flanking copGFP cassette, and introduction of mutation in the knob to make Ad35K++.

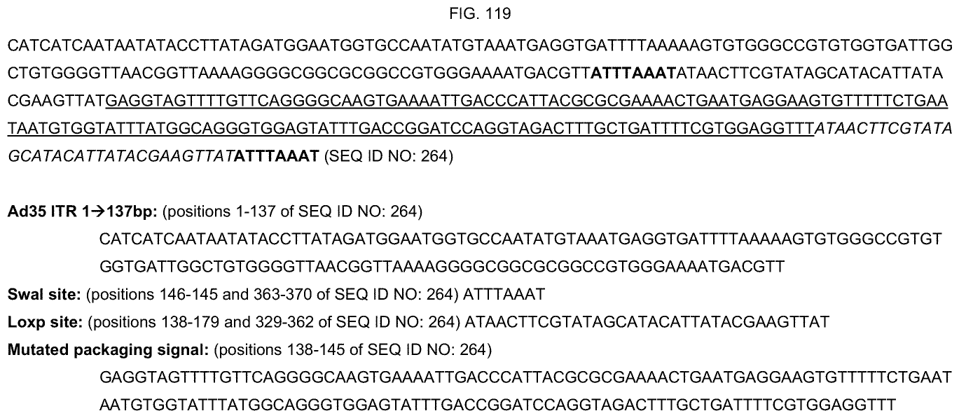

FIG. 119 shows a mutated packaging signal sequence. Residues 1 through 137 are the Ad35 ITR. Text in bold are Swal sites, the Loxp site is italicized, and the mutated packaging signal is underlined. For clarity, these sequences are shown individually in FIG. 119.

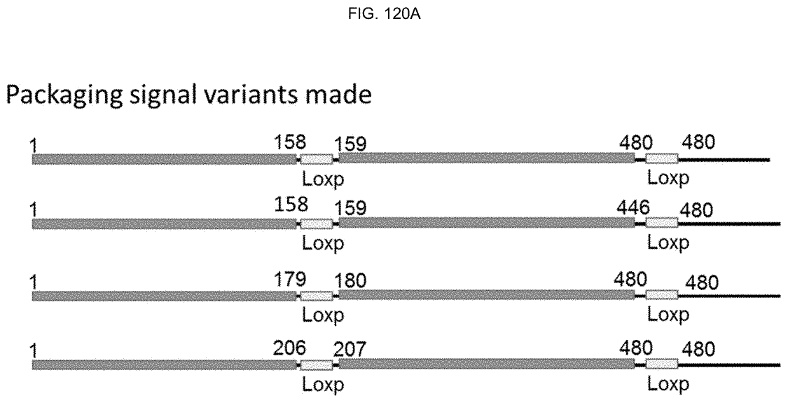

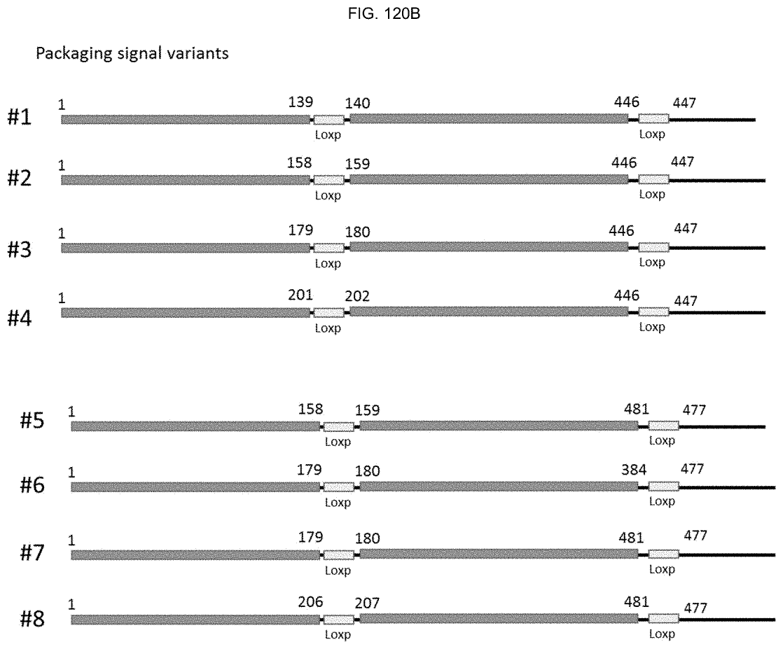

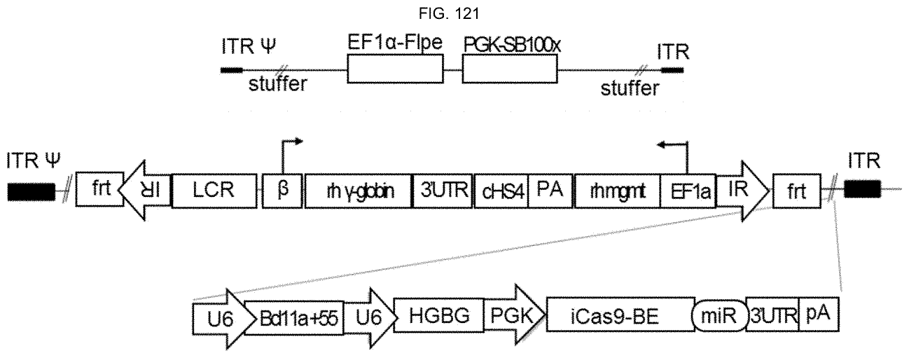

Four Ad35 helper vector packaging signal variants were made (FIG. 120A). The E3 region (27388→30402) was deleted and the CMV-eGFP cassette was located within an E3 deletion, Ad35K++, and eGFP was used instead of copGFP. The LoxP sites in these four packaging signal variants are at the illustrated positions (FIG. 120A). All four helper vectors could be rescued.

FIG. 1208 is a schematic representation of eight additional packaging signal variants, with the specified the LoxP sites.

In certain additional helper vector and packaging signal variants, changes were made to the helper vector in FIG. 120A, such as shortening the E3 deletion (27609→30402).

### Example 5. Targeted Integration and High-Level Transgene Expression in AAVS1 Transgenic Mice after Ex Vivo and In Vivo Hematopoietic Stem Cells Transduction with HDAd5/35++ Vectors

At least some of the information contained in this example was published in Li et al. (Mol Ther., 27(12): 2195-2212, 2019; e-pub Aug. 19, 2019).

Current hematopoietic stem cell gene therapy in patients use lentivirus vectors for gene delivery (Nadini, EMBO Mol Med, 11, 2019; Wang et al., Genome Res, 17, 1186-1194, 2007). Lentivirus vectors efficiently integrate in the human genome with a strong bias toward actively transcribed genes. This semi-random integration pattern entails a risk of perturbing the expression of neighboring genes, including cancer-related genes. A major goal in the field is therefore to target transgene integration to a preselected site. A number of “safe harbors” for targeted integration into the human genome have been suggested (e.g. AAVS1 and CCR5) (Papapetrou et al., Nat Biotechnol, 29, 73-78, 2011). Among the criteria for a safe harbor site are: (i) distance of >50 kb from 5′ end of any gene, (ii) distance of >300 kb from cancer-related genes, (iii) distance of >300 kb from any microRNA, (iv) outside a gene transcription unit, and (v) outside of ultra-conserved regions. The AAVS1 locus in chromosome 19 is used by wild-type AAV for integration mediated by the virus-encoded protein Rep78 that recognizes a specific motif (RBS) within the AAVS1 site (Muzyczka, Curr Top Microbiol Immunol, 158, 97-129, 1992, Huser et al., PLoS Pathog, 6, e1000985, 2010). Because a large proportion of the human population has encountered AAV, as evidenced by detectable antibodies against some AAV serotypes, but without any discernable pathology, it was concluded that integration into AAVS1 may be safe (Henckaerts et al., Future Virol, 5, 555-574, 2010). Furthermore, this locus contains a DNAse I hypersensitive site and an insulator that maintain an open chromatin conformation in CD34+ and iPS cells (van Rensburg et al., Gene Ther, 20, 201-214, 2013, Lombardo et al., Nat Methods, 8, 861-869, 2011, Ogata et al., J Virol, 77, 9000-9007, 2003). This allows for better access of genome editing tools, and on the other hand should, support high-level transgene expression (van Rensburg et al., Gene Ther, 20, 201-214, 2013, Voigt et al., J Mol Med, 86, 1205-1219, 2008).

Targeted transgene integration can be achieved via homology-directed repair (HDR) (Lombardo et al., Nat Med, 20, 1101-1103, 2014). Following cleavage by an engineered site-specific nuclease, DNA double-strand breaks are resolved through non-homologous end joining (NHEJ), an error-prone DNA repair pathway that typically leads to variable insertions or deletions (indels), or HDR, which repairs DNA by copying a homologous donor template. Delivery of exogenous DNA flanked by DNA homologous to the genomic sequence around the break site can lead to incorporation of the exogenous sequence in a site-specific manner.

Current approaches to achieve targeted integration are based on electroporation of HSCs in vitro with endonuclease-encoding mRNA and donor plasmid DNA (Blair et al., J Vis Exp, e53583, 2016, Dreyer et al., Biomaterials, 69, 191-200, 2015; Kuhn et al., Sci Rep, 7, 15195 2017; Li et al., Mol Med Rep, 15, 1313-1318, 2017), integration-deficient lentivirus vectors (IDLV) (Lombardo et al., Nat Med, 20, 1101-1103, 2014; Rio et al., EMBO Mol Med, 6, 835-848, 2014) or rAAV6 vectors (De Ravin et al., Nat Biotechnol, 34:424-429, 2016, Hung et al., Mol Ther, 26, 46-467, 2018; Johnson et al., Sci Rep, 8:12144, 2018). Helper-dependent adenovirus (HDAd5/35++) vectors were developed to deliver designer integrases (Li et al., Blood, 1431, 2915-2928, 2018, Saydaminova et al., Mol Ther Methods Clin Dev, 1, 14057, 2015) and, in this study, donor templates. HDAd5/35++ vectors target human CD46, a receptor that is expressed on primitive HSCs (Richter et al., Blood, 128, 2206-2217, 2016). The ability of HDAd5/35++ vectors to efficiently deliver their genomes into the nucleus of non-dividing cells allows for high amounts of donor DNA, a prerequisite for efficient targeted integration. Because HDAd5/35++ and HDAd35 vectors can carry up to 30 bp of foreign DNA, they can accommodate long stretches of donor sequences that are homologous to the given target site. This should increase the efficacy of gene targeting by homologous recombination, which directly correlates with the length of the homology region (Balamotis et al., Virology, 324, 229-237, 2004, Ohbayashi et al., Proc Nati Acad Sci USA, 102, 13628-13633, 2005, Suzuki et al., Proc Nati Acad Sci USA, 105, 13781-13786, 2008). Because these vectors are easy to produce at high yields and have a strong HSC tropism, they have been employed for in vivo HSC transduction (Richter et al., Blood, 128, 2206-2217, 2016). The central idea of the approach is to mobilize HSCs from the bone marrow using G-CSF/AMD3100, and while they circulate at high numbers in the periphery, transduce them with intravenously injected HDAd5/35++ vectors. Transduced cells return to the bone marrow where they persist long-term. The safety and efficacy of the approach was previously demonstrated in CD46 transgenic mouse models for hemoglobinopathies either by CRISPR/Cas9-mediated reactivation of endogenous fetal globin (Li et al., Blood, 1431, 2915-2928, 2018) or by fetal globin gene addition using a hyperactive Sleeping Beauty Transposase (SB100x) that mediates efficient random transient integration (Wang et al., J Clin Invest, 129, 598-615, 2019). While SB100x-mediated transgene integration is theoretically safer than the quasi-random integration of lentivirus vectors, it still raises concerns with regards to transgene silencing, undesired effects on neighboring genes, and genomic rearrangements. The goal of this study was therefore to modify the HDAd5/35++-based in vivo HSC transduction approach for targeted integration into AAVS1.

A sequence homologous to the human AAVS1 locus is absent in rodents (Samulski et al., EMBO J, 10, 3941-3950, 1991). Two transgenic rodent models have been reported previously, which contain either a 3.5-kb fragment of the AAVS1 locus (7 copies head-to-tail in rats) in the rat or mouse genome (X-chromosome) (Rizzuto et al., J Virol, 73, 2517-2526, 1999). A study showed that the open chromatin structure of AAVS1 is maintained in transgenic mice (Young et al., J Virol, 74, 3953-3966, 2000). Jackson Laboratories distributes AAVS1 transgenic mice (Bakowska et al., Gene Ther, 10, 1691-1702, 2003). Jackson Labs' website states that these mice contain five copies of an 8.2 kb human AAVS1 locus fragment inserted into a single genomic site. To make AAVS1 transgenic mice suitable for transduction with HDAd5/35++ vectors, they were crossed with mice that were transgenic for the human CD46 locus (Kemper et al., Clin Exp Immunol, 124, 180-189, 2001). All animal studies were performed with AAVS1/CD46+/+ mice.

Materials and Methods.

Cells: CD34⁺ cells from G-CSF-mobilized adult donors were obtained. Cells were recovered from frozen stocks and incubated overnight in StemSpan H3000 (STEMCELL Technologies, Vancouver, Canada) with penicillin/streptomycin, Flt3 ligand (F1t3L, 25 ng/ml), interleukin 3 (10 ng/ml), thrombopoietin (TPO) (2 ng/ml), and stem cell factor (SCF) (25 ng/ml). Cells were transduced with HDAd vectors at a MOI of 2000 vp/cell and analyzed as indicated. HUDEP-2 cells. HUDEP-2 cells (Kurita et al., PLoS One, 8, e59890, 2013) were also obtained. HUDEP-2 cells were cultured in the presence of SCF, EPO, Doxycycline and Dexamethasone as previously described (Canver et al., Nature, 527, 192-197, 2015). The cells were transduced with the HDAd vectors at a MOI of 500-1000 vp/cell and analyzed as indicated.

HDAd5/35++ vectors: HDAd-SB, HDAd-IR-GFP/mgmt, and HDAd-IR-γ-globin/mgmt have been described before (Li et al., Mol Ther Methods Clin Dev, 9, 142-152, 2018, Wang et al., Mol Ther Methods Clin Dev, 8, 52-64, 2018). For the cloning of the HDAd-CRISPR vector, sgRNA (SEQ ID NO: 207) (Mali et al., Science, 339, 823-826, 2013) targeting the human AAVS1 locus was synthesized, annealed and inserted into the Bbsl site of pSPgRNA (Addgene, Cambridge, Mass.), generating pSP-sgAAVS1. A Cas9 coding sequence amplified from pLentiCRISPRv2 (Addgene), U6sgAAVS1 fragments released by BamHI digestion of pSP-sgAAVS1, and a previously described microRNA targeting region (miR-183/218) (Saydaminova et al., Mol Ther Methods Clin Dev, 1, 14057, 2015) were sequentially cloned into the EcoRV-NotI, BamHI and NotI sites of pBS-T-EF1α (Saydaminova et al., Mol Ther Methods Clin Dev, 1, 14057, 2015), forming pBST-sgAAVS1-miR. To obtain the recombinant adenoviral plasmids, an 8 kb cassette starting from the U6 promoter to the SV40 polyA signal sequence was amplified from pBST-sgAAVS1-miR and ligated with NheI-XmaI digested pHCA (Sandig et al., Proc Natl Acad Sci USA, 97, 1002-1007, 2000) by Gibson assembly (New England Biolabs), generating the corresponding pHCA-sgAAVS1-miR plasmid.

For the construction of the HDAd-GFP-donor vector, two 0.8 kb homology arms (HA) immediately flanking the AAVS1 CRISPR cutting site were synthesized as gBlocks (IDT, San Jose, Calif.). One 23 bp sgAAVS1 with PAM sequence was included upstream of the 5′HA and downstream of 3′HA, respectively, to mediate the release of the donor cassette. A EF1α-mgmt-2A-GFP-pA fragment was synthesized by GenScript (Nanjing, China), and ligated with the two 5′HAs by overlap PCR, forming sgAAVS1-5′HA-Ef1α-mgmt-2A-GFP-pA-3′HA-sgAAVS1 which was subsequently inserted into the XmaI site of pHCA (Sandig et al., Proc Natl Acad Sci USA, 97, 1002-1007, 2000), generating GFP donor vector pHCA-AAVSI-GFP-mgmt.

The cloning of the HDAd-globin-donor vector involved 3 steps. Step 1) The 11.8 kb LCR-globin-mgmt cassette was released from pHM5-FR-IR-LCR-globin-mgmt (Li et al., Mol Ther Methods Clin Dev, 9, 142-152, 2018) by EcoRV-KpnI digestion and ligated with a 2.8 kb plasmid backbone amplified from pBS-Z (Saydaminova et al., Mol Ther Methods Clin Dev, 1, 14057, 2015), resulting in pBS-LCR-globin-mgmt. Two 1.8 kb HAs immediately adjacent to the AAVS1 CRISPR cutting site were PCR amplified from genomic DNA isolated from bone marrow cells of AAVS1-tg mice using primers containing the 23 bp sgAAVS1 with PAM sequence. The 5′ and 3′ side HAs were sequentially inserted into the EcoRV and KpnI sites, respectively, of pBS-LCR-globin-mgmt, generating pBS-AAVS1-globin-mgmt. Step 2) The nt1588-12121 region of pHCA was deleted by EcoRI digestion and self-ligation, generating pHCAS1. The original PacI site in pHCAS1 was destroyed by inserting two annealed oligo sequences. A new PacI cloning site was created at a BstBI site, getting pHCAS1-MCS. This cloning site was designed in such a way that two 15 bp homologous regions are exposed upon PacI digestion. The size of pHCAS1-MCS was further reduced by removing the 1.5 kb NheI fragment, resulting in pHCAS1S-MCS. Step 3) Following PacI digestion of the two final constructs from the above two steps, the products were recombined by Gibson Assembly, generating the globin donor vector pHCA-AAVS1-globin-mgmt.

For the production of HDAd5/35++ vectors, corresponding plasmids were linearized with PmeI and rescued in 116 cells (Palmer et al., Mol Ther, 8, 846-852, 2003) with Ad5/35++-Acr helper vector (Li et al., 2018. Blood, 1431, 2915-2928) as described in detail elsewhere (Palmer et al., Mol Ther, 8, 846-852, 2003). Helper virus contamination levels were found to be <0.05%. Titers were 6-12×10¹² vp/ml. All HDAd vectors used in this study contain chimeric fibers composed of the Ad5 fiber tail, the Ad35 fiber shaft, and the affinity-enhanced Ad35++fiber knob (Wang et al., J Virol, 82, 10567-10579, 2008).

Mismatch sensitive nuclease assay T7E1 assay. Genomic DNA was isolated as previously described (Miller et al., Nat Biotechnol, 25, 778-785, 2007). Genomic segments encompassing the AAVS1 target site were amplified by KOD Hot Start DNA Polymerase (MilliporeSigma, Burlington, Mass.) using the following primers: AAVS1 forward (SEQ ID NO: 208); reverse (SEQ ID NO: 209). PCR products were hybridized and treated with 2.5 Units of T7E1 (NEB) for 20 minutes at 37° C. Digested PCR products were resolved by 6% TBE PAGE (Bio-Rad) and stained with ethidium bromide. Band intensity was analyzed using ImageJ software. % cleavage=(1-sqrt(parental band/(parental band+cleaved bands)))×100%

Next generation sequencing: For deep sequencing of insertion/deletions (indels), a 250-bp region surrounding the predicted AAVS1 cleavage site was amplified and sequenced the products using an Illumina system. Genomic DNA was isolated as previously described (Saydaminova et al., Mol Ther Methods Clin Dev, 1, 14057, 2015). A 249 bp genomic region encompassing the AAVS1 target site was amplified using the following primers: AAVS1 forward (SEQ ID NO: 210); reverse (SEQ ID NO: 211). After cleaning-up the amplicon using AMPure XP Beads (Beckman Coulter, Indianapolis, Ind.), dA-tailing was performed using Klenow fragment. Illumina-compatible adaptors were ligated with the product by T4 ligase (New England Biolabs). A unique barcode sequence was introduced by PCR to allow sequencing multiple samples on the same sequencing run. Each step was followed by purification with AMPure XP Beads. The final libraries were quantified by Qubit (Invitrogen) and tested on an Agilent 2100 Bioanalyzer to determine average size of the amplicons. The amplicons were pooled at equal molarity and deep sequenced on an Illumina MiSeq system. 10⁵ reads per amplicon were generated to adequately probe the types of mutations. Sequencing data were aligned to the AAVS1 reference sequence using the Cas-Analyzer online tool (available at rgenome.net/cas-analyzer/#!) (Park et al., Bioinformatics, 33, 286-288, 2017, a JavaScript-based implementation for NGS data analysis.

Flow cytometry: Cells were resuspended at 1×10⁶ cells/100 μL in FACS buffer (PBS supplemented with 1% heat-inactivated FBS) and incubated with FcR blocking reagent (Miltenyi Biotech, Auburn Calif.) for ten minutes on ice. Next the staining antibody solution was added in 100 μL per 10⁶ cells and incubated on ice for 30 minutes in the dark. After incubation, cells were washed once in FACS buffer. For secondary staining the staining step was repeated with a secondary staining solution. After the wash, cells were resuspended in FACS buffer and analyzed using a LSRII flow cytometer (BD Biosciences, San Jose, Calif.). Debris was excluded using a forward scatter-area and sideward scatter-area gate. Single cells were then gated using a forward scatter-height and forward scatter-width gate. Flow cytometry data were then analyzed using FlowJo (version 10.0.8, FlowJo, LLC). For flow analysis of LSK cells, cells were stained with biotin-conjugated lineage detection cocktail (Miltenyi Biotec, San Diego, Calif.) and antibodies against c-Kit and Sca-1 as well as APC-conjugated streptavidin. Other antibodies from eBioscience (San Diego, Calif.) included anti-mouse LY-6A/E (Sca-1)-PE-Cyanine7 (clone D7), anti-mouse CD117 (c-Kit)-PE (Clone 2B8), anti-mouse CD3-APC (clone 17A2), anti-mouse CD19-PE-Cyanine7 (clone eBio1D3), and anti-mouse Ly-66 (Gr-1)-PE, (clone RB6-8C5). Other antibodies from Miltenyi Biotec included anti-human CD46-APC (clone: REA312). Anti-mouse Ter-119-APC (clone: Ter-119) was from BioLegend (San Diego, Calif.).

Intracellular staining of human γ-globin was performed using PE-conjugated anti-human γ-globin antibody from Santa Cruz (clone 51.7). The Fix & Perm cell permeabilization kit from Invitrogen was used according to manufacturer's instructions.

Real-time reverse transcription PCR: Total RNA was extracted from 50-100 μL blood by using TRIzol™ reagent (Thermo Fisher Scientific) following the manufacture's phenol-chloroform extraction method, then reverse transcribed to generate cDNA using Quantitect reverse transcription kit from Qiagen. Potential genomic DNA contamination was eliminated by treatment of the RNA samples with gDNA wipe-out reagents provided in the kit. Comparative real-time PCR was performed using Power SYBR Green PCR master mix (Applied Biosystems) and ran on a StepOnePlus real-time PCR system (Applied Biosystems). The following primer pairs were used: mouse RPL10 (house-keeping) forward (SEQ ID NO: 189), and reverse (SEQ ID NO: 190); human γ-globin forward (SEQ ID NO: 214), and reverse (SEQ ID NO: 215); mouse β-major globin forward (SEQ ID NO: 193), and reverse (SEQ ID NO: 217).

Globin HPLC: Individual globin chain levels were quantified on a Shimadzu Prominence instrument with an SPD-10AV diode array detector and a LC-10AT binary pump (Shimadzu, Kyoto, Japan). A 38%-58% gradient mixture of 0.1% trifluoroacetic acid in water/acetonitrile was applied at a rate of 1 mL/min using a Vydac C4 reversed-phase column (Hichrom, UK).

Colony forming unit assay. 2500 of Lin− cells were plated in triplicates in ColonyGEL 1202 mouse complete medium (ReachBio, Seattle Wash.) and incubated for 12 days at 37° C. in 5% CO₂ and maximum humidity. Colonies were enumerated using a Leica MS 5 dissection microscope (Leica Microsystems). For colonies derived from HDAd-GFP-donor-transduced mice, GFP positive colonies were counted, picked and analyzed.

Measurement of vector copy number Total DNA from bone marrow cells or single colonies was extracted by PureLink Genomic DNA Mini Kit (Invitrogen). Viral DNA extracted from HDAd-GFP-donor or HDAd-globin-donor was serially diluted and served as standard curve. qPCR was conducted duplicate using the power SYBR Green PCR master mix on a StepOnePlus real-time PCR system (Applied Biosystems). 5 ng DNA was used for a 10 μL reaction. The following primer pairs were used: GFP forward (SEQ ID NO: 218), and reverse (SEQ ID NO: 219); and mgmt forward (SEQ ID NO: 220), and reverse (SEQ ID NO: 221). Human γ-globin primers were described in the paragraph of Real-time reverse transcription PCR.

Localization of AAVS1 locus in AAVS1 transgenic mice. TLA library was prepared as described previously (de Vree et al., Nat Biotechnol, 32, 1019-1025, 2014). Briefly, formaldehyde crosslinked DNA from total bone marrow cells were digested with NlaIII. After ligation and reverse crosslinking, DNA was purified. This product was further digested with NspI and ligated to obtain circular chimeric DNA of 2 kb. Chimeric DNA was PCR amplified using AAVS1 specific TLA primers: forward (SEQ ID NO: 222), and reverse (SEQ ID NO: 223). TLA libraries from PCR amplified product was prepared using Illumina Nextera XT NGS kit according to manufacturer's protocol. Paired end sequencing was performed on NovaSeq. TLA protocol leads to reshuffling of DNA, thus reads were aligned using split-read aware aligner BWA (Li et al., Bioinformatics, 26, 589-595, 2010) using settings: bwasw-b 7 as suggested previously (see online at github.com/Cergentis/Cergentis_common) (Vain-Hom et al., 2017. Nucleic Acids Res, 45, e62). These aligned bam files were converted to RPKM normalized bigwig files using deepTools (Ramirez et al., Nucleic Acids Res, 42, W187-191, 2014). Genome wide distribution was visualized using WashU epigenome browser (Zhou et al., Nat Methods, 8, 989-990, 2011).

Southern Blot. Genomic DNA from mouse bone marrow was digested with either EcoRl or Blp1 and subjected to Southern blot with either an AAVS1- or GFP-specific probe labelled with ³²P using the Prime-It RmT Random Primer labeling kit (Agilent Technologies). Non-incorporated ³²P dCTP was removed by centrifugation through MicroSpin G25 columns (GE Healthcare). Hybridization was performed in PerfectHyb Plus hybridization buffer (Sigma). Blots were exposed to Amersham Hybond-XL films (GE Healthcare).

Inverse PCR: Junctions in total bone marrow cells, single colonies, HUDEP-2 cell mixture or clones were analyzed by inverse PCR as described elsewhere with modifications (Wang et al., J Virol, 79, 10999-11013, 2005). Briefly, genomic DNA was isolated by incubating with genomic DNA lysis buffer (100 mM Tris-CI (pH 8.0), 50 mM EDTA, 1% (w/v) SDS, and 400 μg/mL Proteinase K) at 55° C. overnight with shaking, followed by Phenol-Chloroform extraction, precipitation with isopropanol, and wash with 70% ethanol. The DNA samples were dissolved in 10 mM Tris/HCL buffer (pH 8.5). 5 μg of DNA was digested with 30 U NcoI in 50 μL reaction at 37° C. for 5 hours. After heat-inactivation and clean-up, the digested DNA was treated with 2.5 μL T4 ligase (New England Biolabs, M0202L) in 500 μL reaction buffer at 16° C. overnight for intramolecular ligation. Following heat-inactivation and clean-up, the re-ligated product was used for inverse PCR using KOD Hot Start DNA Polymerase. The following primers were used: EF1a, forward (SEQ ID NO: 224), and reverse (SEQ ID NO: 225); pA forward (SEQ ID NO: 226), and reverse (SEQ ID NO: 227); HS4 forward (SEQ ID NO: 228); and reverse (SEQ ID NO: 229). The Ef1a, and pA primer pairs were used for analyzing 5′ and 3′ junctions of GFP donor vector-treated samples, respectively. The HS4 and EF1α primer pairs were used for analyzing 5′ and 3′ junctions of globin donor vector-treated samples, respectively. PCR amplicons were gel purified, cloned, sequenced and aligned to identify the integration sites.

In-Out PCR: Genomic DNA was extracted as described in the section of Inverse PCR. 5 ng genomic DNA was directly used as template for In-Out PCR by KOD Hot Start DNA Polymerase in a 25 μl of reaction. The following PCR program was used: 94° C. 2 min; 5 cycles of 98° C. 10 sec, 66° C. 30 sec and 68° C. 1.5 min; 5 cycles of 98° C. 10 sec, 63° C. 30 sec and 68° C. 1.5 min; 15 cycles of 98° C. 10 sec, 60° C. 30 sec and 68° C. 1.5 min; 68° C. 5 min. Primers used are In-Out P1 (SEQ ID NO: 230), In-Out P2 (SEQ ID NO: 231), and In-Out P3 (SEQ ID NO: 232). The products were resolved in a 1% Agarose gel. One single 1.6 kb band indicates biallelic targeted integration; one 1.6 kb plus one 2.0 kb band indicates monoallelic targeted integration; one single 2.0 kb band indicates potential off-target integration.

In silico prediction of off-target cleavage sites: Off-target sites of the AAVS1 guide sequence in human or mouse genome were predicted using the online tool: available at sanger.ac.uk/htgt/wge/find_off_targets_by_seq.

Animal studies: All experiments were conducted with approval from the controlling Institutional Review Board and IACUC. Mice were housed in specific-pathogen-free facilities. AAVS1 transgenic mice (C3; B6-Tg(AAVS1)A1Xob/J) (The Jackson Laboratory) were recovered from cryopreserved embryos of mice as described in Bakowska et al. (Gene Ther, 10, 1691-1702, 2003). Mice are hemizygous for the human AAVS1 locus. AAVS1 transgenic mice were crossed with human CD46+/+ mice to obtain AAVS1^{+/-}/CD46^{+/-} mice for ex vivo studies and AAVS1^{+/-}/CD46+/+ mice for in vivo HSC transduction studies. The following primers were used for genotyping of CD46 mice: forward (SEQ ID NO: 233), and reverse (SEQ ID NO: 234). Mice homozygous or heterozygous for CD46 were identified by different intensity of CD46 expression on PBMCs detected by flow cytometry. Genotyping of AAVS1 transgene was performed by PCR according to Jackson Labs' recommended protocol.

Bone marrow Lin⁻ cell transplantation: Recipients were female C57BL/6 mice, 6-8 weeks old. On the day of transplantation, recipient mice were irradiated with 1000 Rad. Four hours after irradiation 1×10⁶ Lin⁻ cells were injected intravenously through the tail vein. This protocol was used for transplantation of ex vivo transduction Lin⁻ cells and for transplantation into secondary recipients.

HSC mobilization and in vivo transduction: This procedure was described previously (Richter et al., Blood, 128, 2206-2217, 2016). Briefly, HSCs were mobilized in mice by s.c. injections of human recombinant G-CSF (5 μg/mouse/day, 4 days) (Amgen Thousand Oaks, Calif.) followed by an s.c. injection of AMD3100 (5 mg/kg) (Sigma-Aldrich) on day 5. In addition, animals received Dexamethasone (10 mg/kg) i.p. 16 h and 2 h before virus injection. Thirty and 60 minutes after AMD3100, animals were intravenously injected with HDAd-CRISPR and HDAd-GFP-donor or HDAd-globin-donor through the retro-orbital plexus with a dose of 4×10¹⁰ vp for each virus per injection. Four weeks later, mice were injected with O⁶-BG (15 mg/kg, IP) two times, 30 minutes apart. One hour after the second injection of O⁶-BG, mice were injected with BCNU (5 mg/kg, IP). The BCNU dose was increased in the second cycle to 10 mg/kg. Both BCNU and O⁶-BG were from Sigma-Aldrich.

Statistical analyses: For comparisons of multiple groups, one-way and two-way analysis of variance (ANOVA) with Bonferroni post-testing for multiple comparisons was employed. Statistical analysis was performed using Graph Pad Prism version 6.01 (GraphPad Software Inc., La Jolla, Calif.).

Results

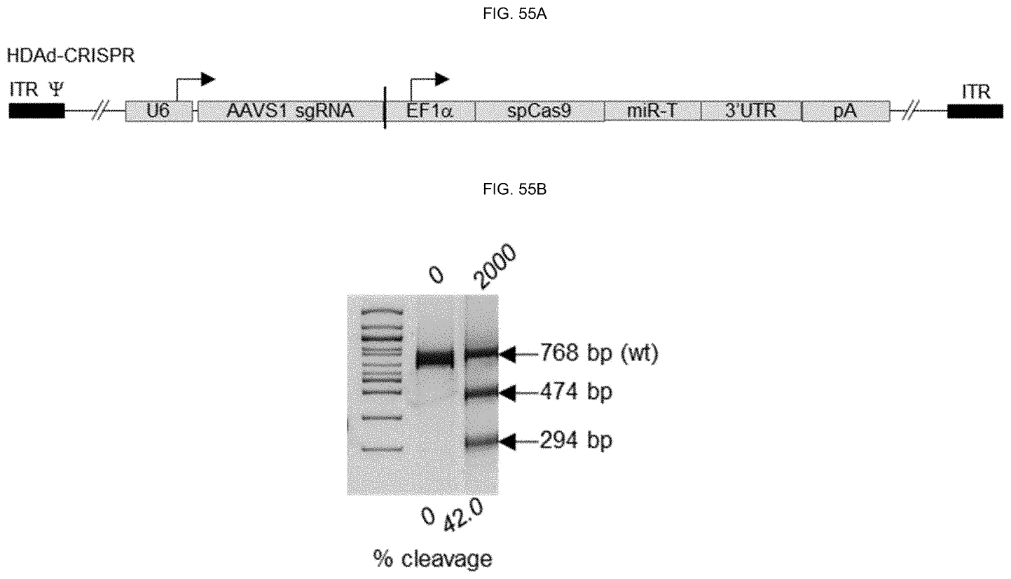

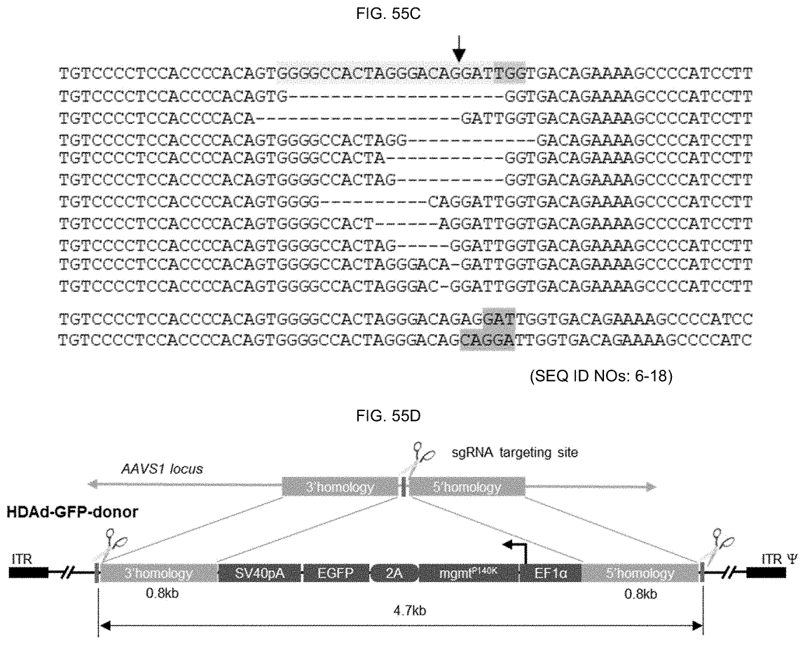

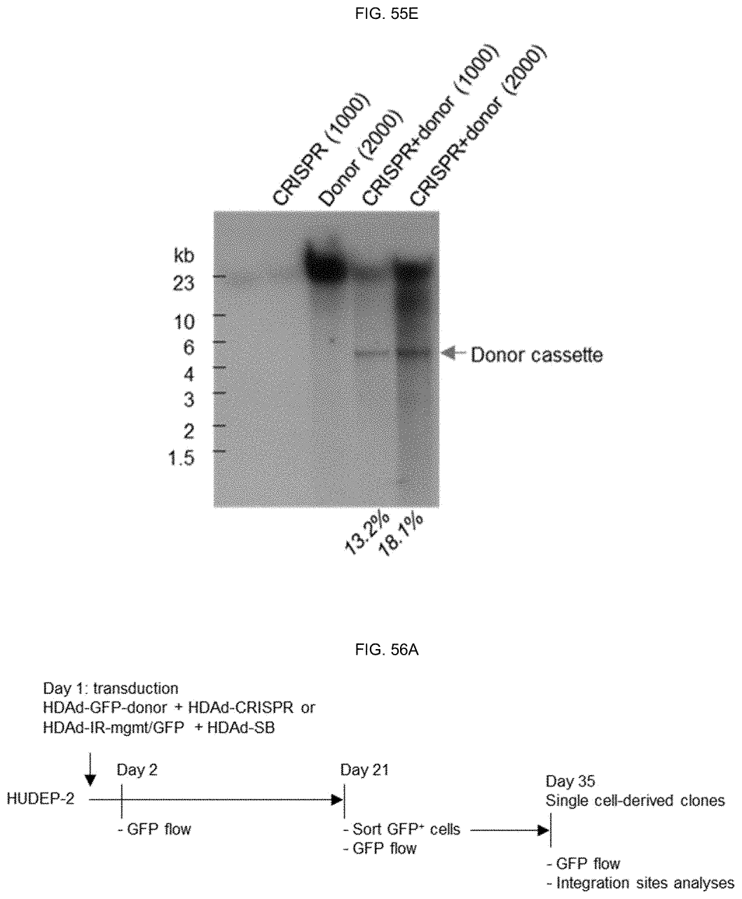

Design of HDAd-CRISPR and HDAd-donor vectors. A HDAd5/35++ vector expressing a CRISPR/Cas9 was created. The vector is capable of creating ds DNA breaks within the AAVS1 locus (FIG. 55A). Previous studies demonstrated that site-specific integration into this locus allowed for robust transgene expression without side-effects in primary human cells (Lombadro et al., Nat Methods, 8, 861-869, 2011). To test the activity of the corresponding HDAd-CRISPR vector human CD34+ cells were transduced, a cell fraction that is enriched for HSCs. AAVS1 site-specific cleavage at day 3 after infection with a frequency of 42% was demonstrated by mismatch sensitive nuclease assay T7E1 assay (FIG. 55B). For deep sequencing of HDAd-CRISPR insertion/deletions (indels), PCR amplification was performed on a 250-bp region surrounding the predicted AAVS1 cleavage site and sequenced the products using an Illumina system (FIG. 55C). 80% of indels were deletions ranging from 1 to 20 bp and only 10% were 1 to 2 bp micro-insertions.

A HDAd5/35++ vector was used as the donor vector. The first HDAd-donor vector contained an expression cassette for GFP and mgmt^{P140K} flanked on both sides by 0.8 kb long regions that are homologous to areas immediately adjacent to the CRISPR/Cas9 target site (FIG. 55D). Linear double-stranded adenoviral genomes are covalently linked with the virus produced “terminal protein—TP” when they enter cells and are translocated to the nucleus (Shenk, Fields Virology, 2:2111-2148, 1996). This same is the case for HDAd5/35++genomes, where the TP is helper virus derived. It is thought that the absence of free DNA ends in the donor greatly diminished HDR (Cristea et al., Biotechnol Bioeng, 110, 871-880, 2013). The sgRNA target sites for the AAVS1 CRISPR were incorporated into the donor vector flanking the donor transgene cassette (FIG. 55D). Co-infection of HDAd-CRISPR and HDAd-GFP-donor should therefore simultaneously create dsDNA breaks in the chromosomal AAVS1 target site and release the donor cassette from the incoming HDAd-donor genome inside the nucleus. IA HDAd-CRISPR-mediated release of the donor cassette from a co-infected HDAd-GFP-donor vector with an efficacy of 13.2 and 18.1% in CD34+ cells at day 2 after infection at a total MOI of 1000 and 2000 vp/cell was demonstrated, respectively (FIG. 55E). This finding also indicates that CRISPR/Cas9 is capable of cleaving double-stranded linear adenoviral DNA, which has implications for anti-viral therapies.

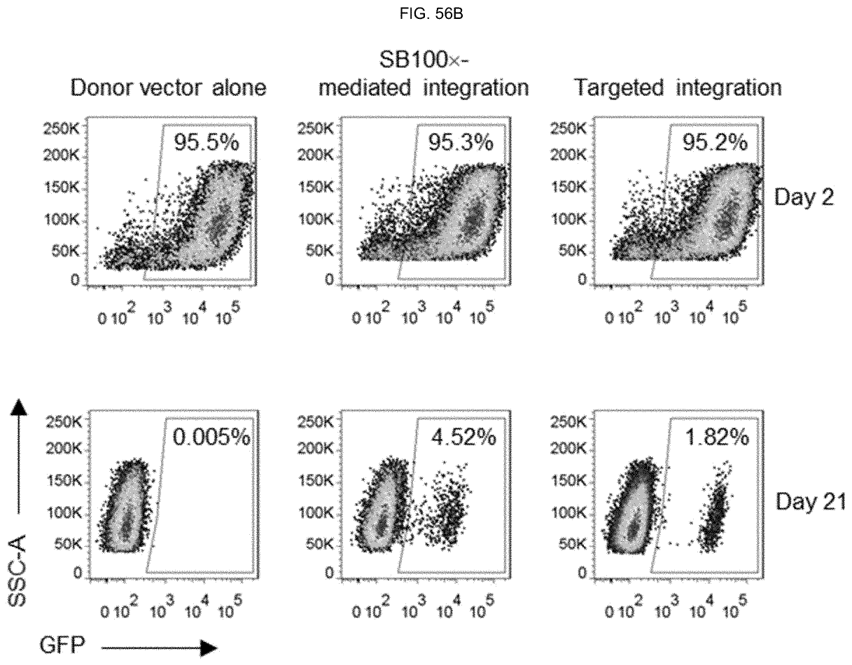

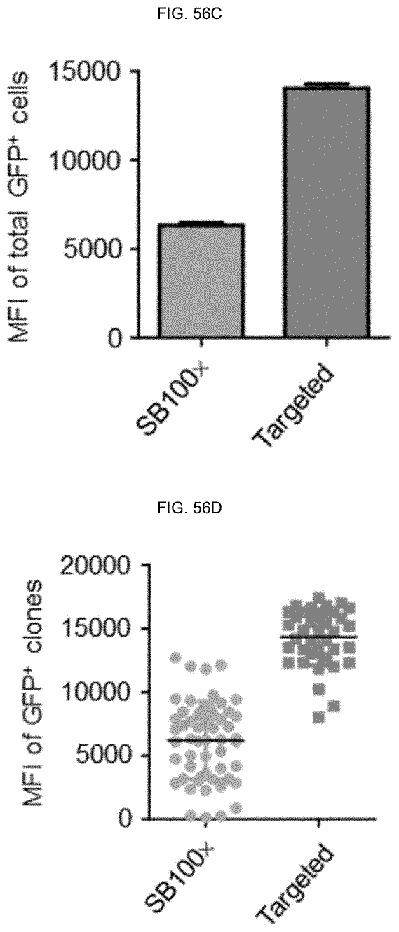

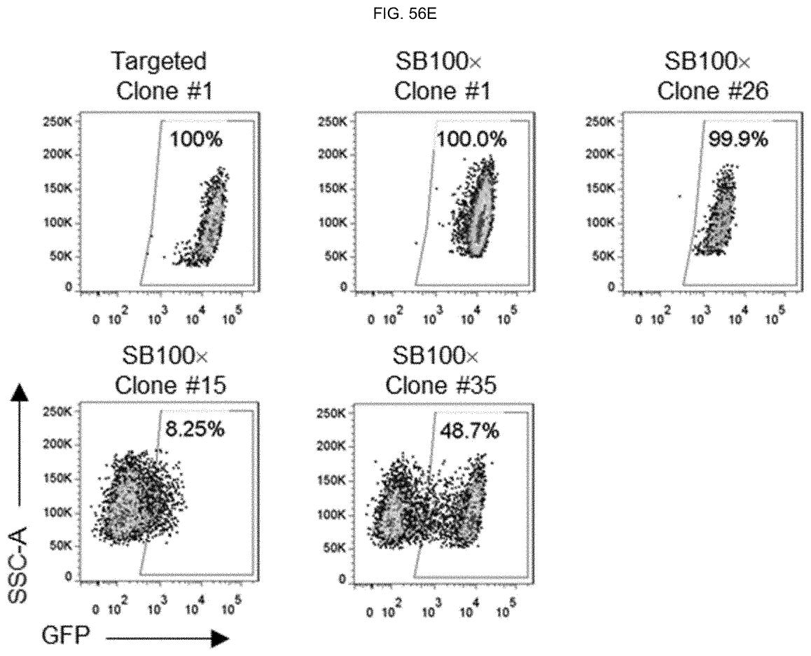

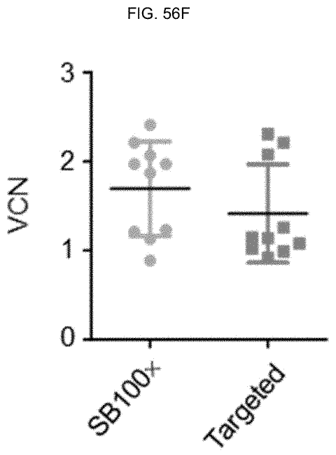

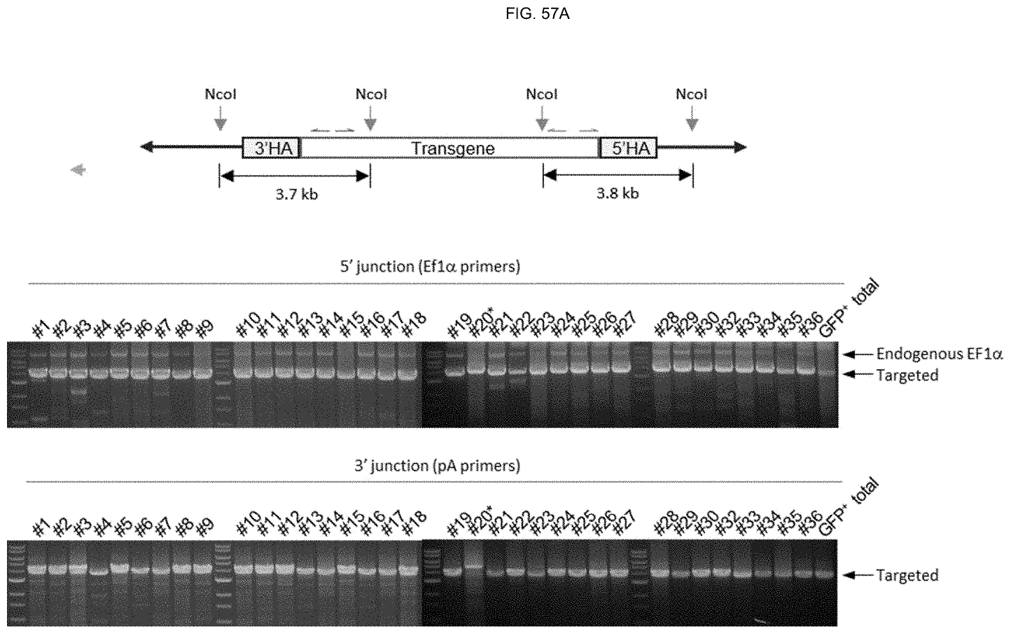

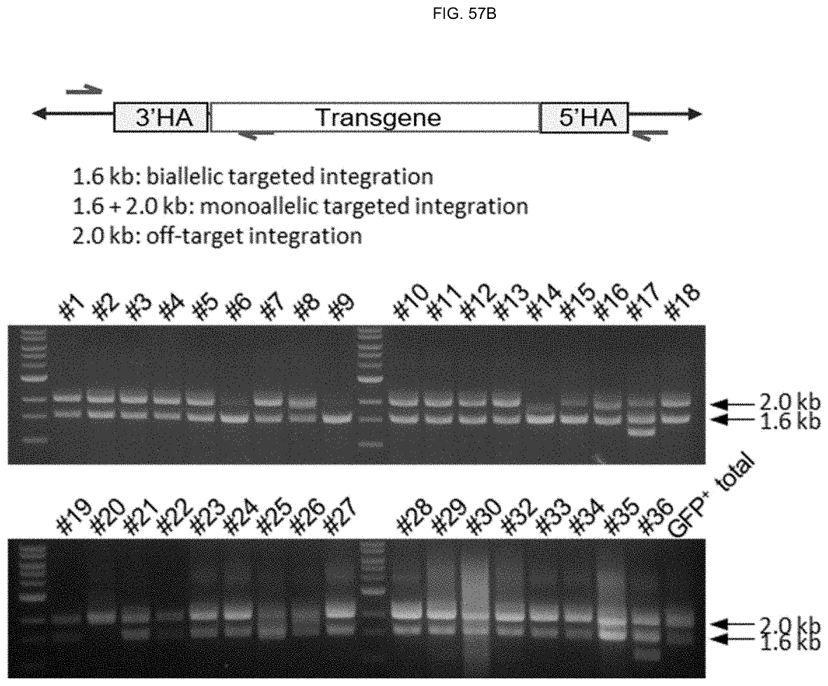

Targeted integration in vitro. First, the HDAd-CRISPR+HDAd-donor vector system was tested for targeted integration in vitro in direct comparison to the SB100x vector system that mediates random integration (FIG. 56A). HUDEP-2 cells, a human erythroid progenitor cell was used. This cell line is diploid and allows for the expansion of single colonies, features that facilitate integration site analysis. GFP flow cytometry performed at day 2 after transduction of HUDEP-2 cells demonstrated similar percentages of GFP-positive cells for the SB100x-mediated and targeted integration systems indicating similar transduction rates (FIG. 56B, upper panel). GFP expression at day 2 is likely to originate from episomal genomes because transduction with HDAd-GFP-donor alone resulted in similar GFP marking. After culturing transduced cells for 21 days, due cell proliferation, episomal genomes disappeared as the absence of GFP expression in the HDAd-GFP-donor alone setting indicates. At day 21, 4.52% and 1.82% of cells were GFP-positive for the SB100x-mediated and targeted integration system, respectively (FIG. 56B, lower panel). This suggests that the SB100x system confers higher stable transduction rates. However, the level of GFP expression, reflected by the Mean Fluorescence Intensity (MFI), was higher in cells transduced with HDAd-CRISPR+HDAd-GFP-donor both in the cell population at day 21 (FIG. 56C) and at the single clone level (FIG. 56D). Vector integration analysis was performed in single clones. Because of the long homology regions flanking the transgene cassette, it was not possible to employ commonly used tools for vector integration site analysis (e.g. LAM-PCR). To demonstrate the presence of vector-cellular DNA junctions, an inverse PCR (iPCR) method was used that involves the endonuclease cleavage of genomic DNA into 4 kb fragments, their circularization, and subsequent PCR with transgene specific primers (Wang et al., J Virol, 79, 10999-11013, 2005). Results showed that all tested 36 colonies derived from HDAd-CRISPR+HDAd-GFP-donor transduced HUDEP-2 cells had transgenes integrated into the AAVS1 site (FIG. 57A). This is in line with the homogeneous high level of transgene expression in clones with targeted integration. In-/out-PCR with AAVS1 and transgene specific primers revealed that integration in 3 out of 36 colonies occurred in both alleles; 31 out of 36 had monoallelic integrations, and 2 apparently had concatemeric integrants (FIG. 57B). In contrast, SB100x-mediated random integration with no preferential targeting of a specific locus (Wang et al., 2019. J Clin Invest, 129, 598-615, Boehme et al., Mol Ther Nucleic Acids, 5, e337, 2016) resulted in varying levels of gene silencing (FIG. 56E). Similar levels of vector copy number were detected in clones with SB100x and targeted integration (FIG. 56F).

In summary, the in vitro studies showed that the HDAd-CRISPR+HDAd-GFP-donor system conferred targeted integration at a high efficiency and resulted in higher GFP expression levels than the SB100x mediated system. The efficacy of stable integration was 40% lower for the targeted system.

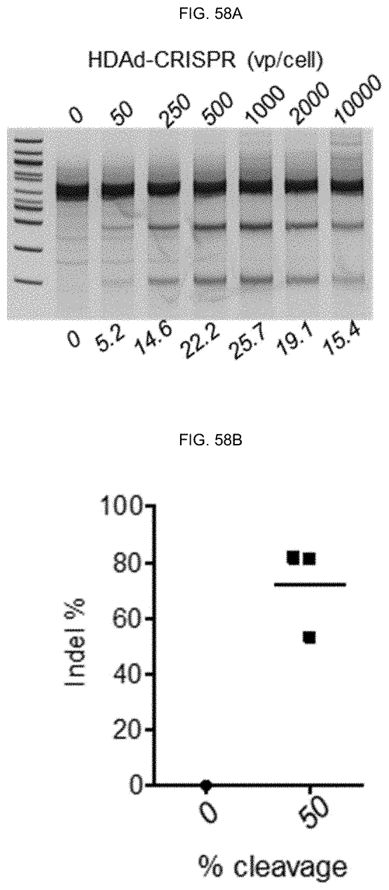

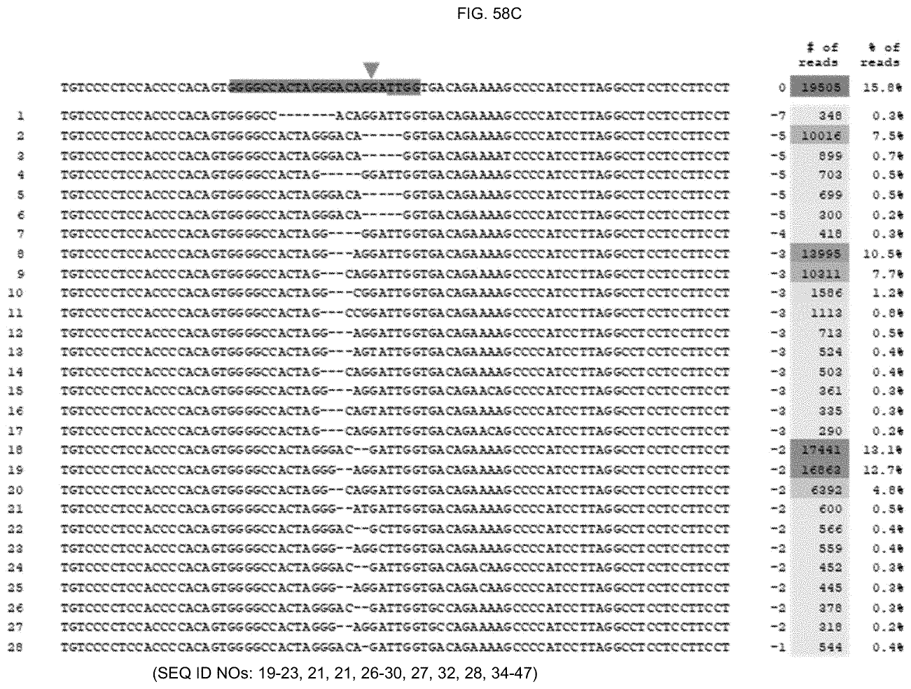

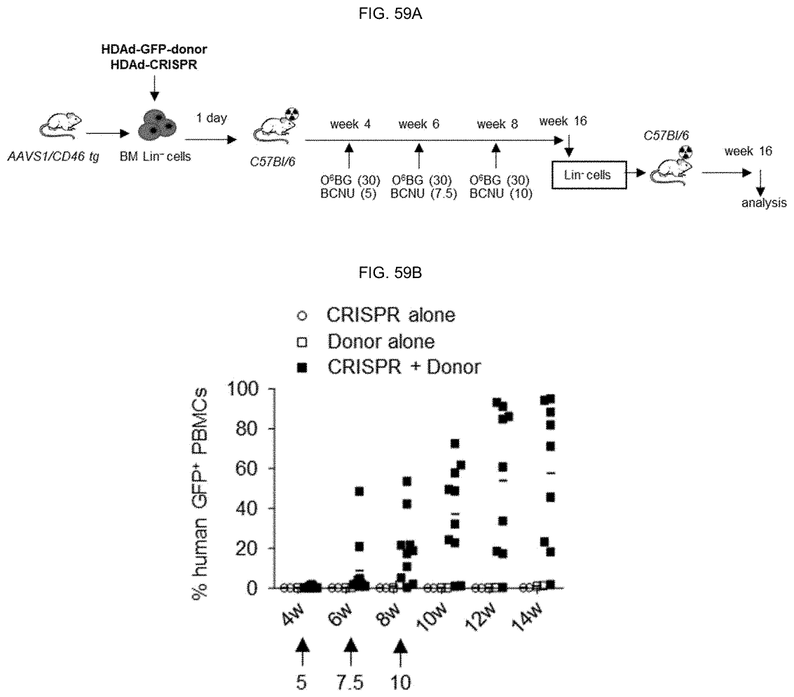

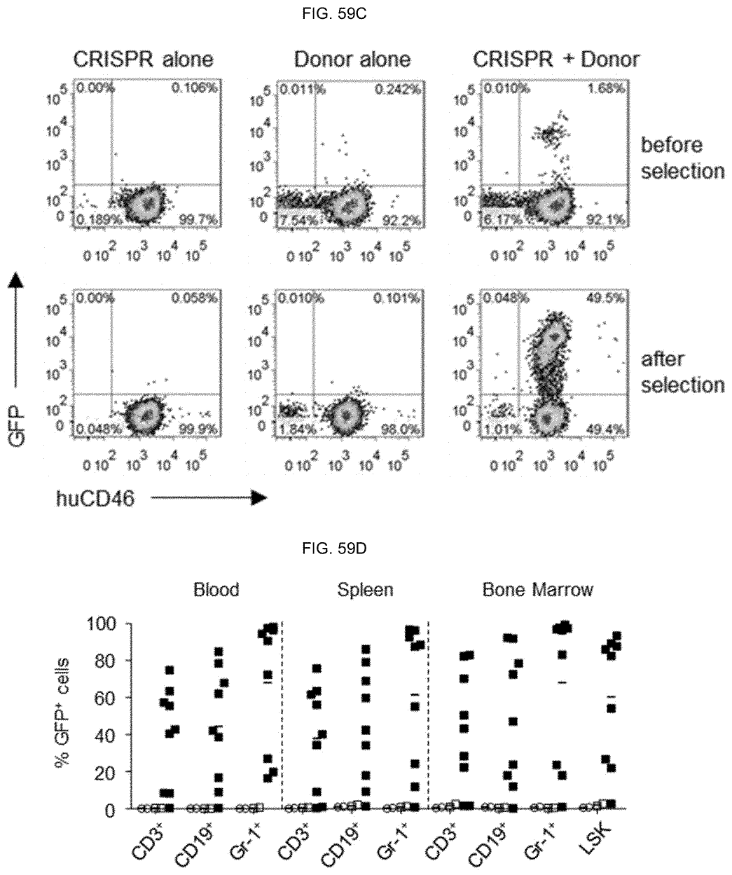

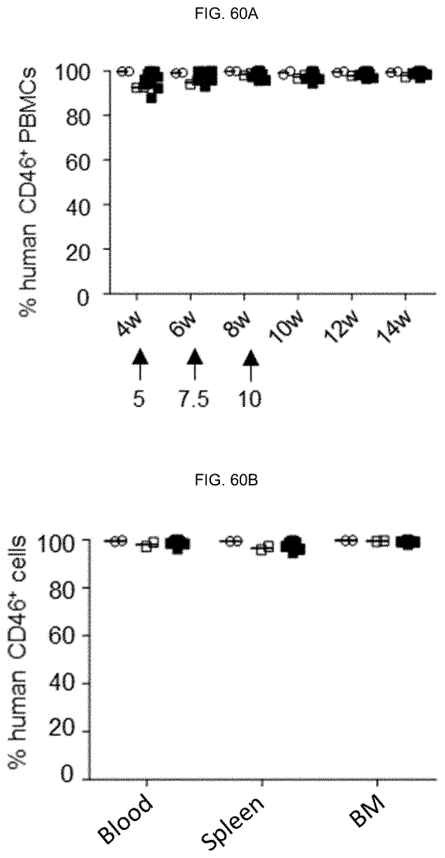

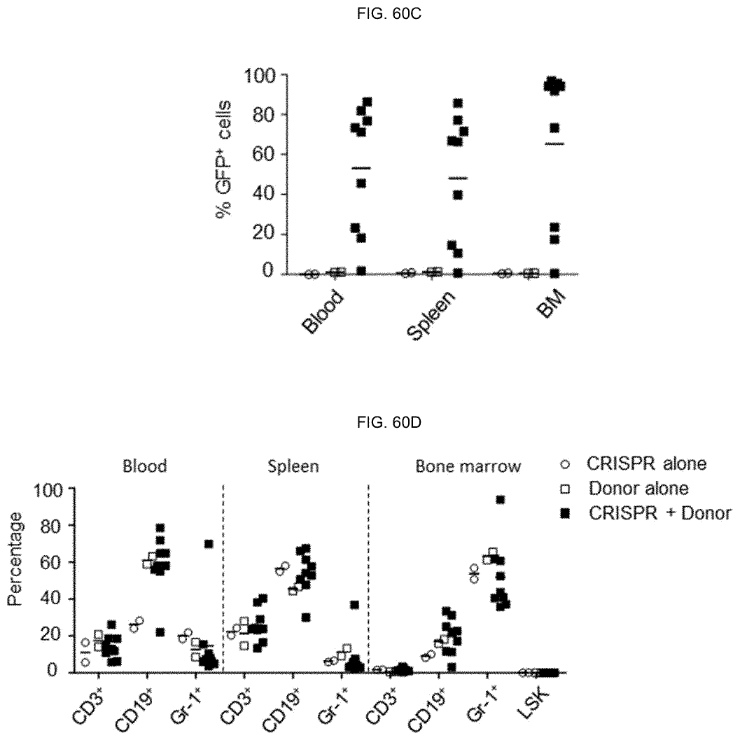

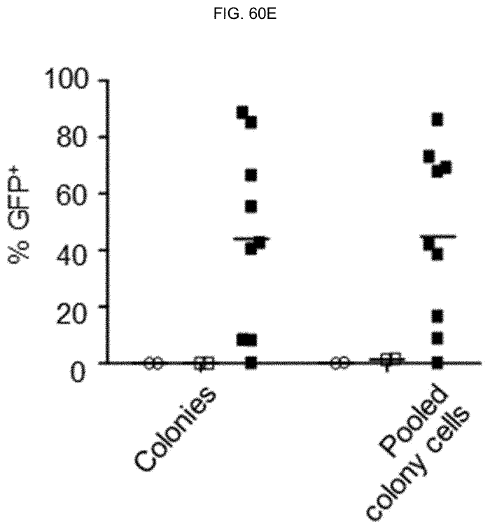

Ex vivo transduction of AAVS1/CD46 HSCs with HDAd-CRISPR+HDAd-GFP-donor and subsequent transplantation into lethally irradiated recipients. Next, the targeted integration system was tested in HSCs from AAVS1/CD46tg mice. The target site cleavage frequency after ex vivo transduction of lineage-negative (Lin⁻) cells, a bone marrow cell fraction enriched for HSCs, was 25% after transduction with the HDAd-CRISPR vector at a MOI of 1000 vp/cell (FIG. 58A). The percentage of insertions/deletions is shown in FIG. 58B at 0% and 50% cleavage. Exemplary sequences are shown in FIG. 58C. AAVS1/CD46 Lin⁻ cells transduced ex vivo with HDAd-CRISPR alone, HDAd-GFP-donor alone, and the combination of both were transplanted into lethally irradiated C57Bl/6 mice, which were then followed for 16 weeks (FIG. 59A). Engraftment of transplanted cells based on human CD46 expression on PBMCs was measured by percent of CD46+ PBMCs at indicated time points. Transduced donor cells expressed CD46 (FIG. 60B), while recipient C57Bl/6 mice did not. Percentage of CD46+ cells in PBMCs (blood), spleen, and bone are shown in FIGS. 60C and 60D. Expression of a GFP marker was also analyzed in colonies and pooled colony cells.

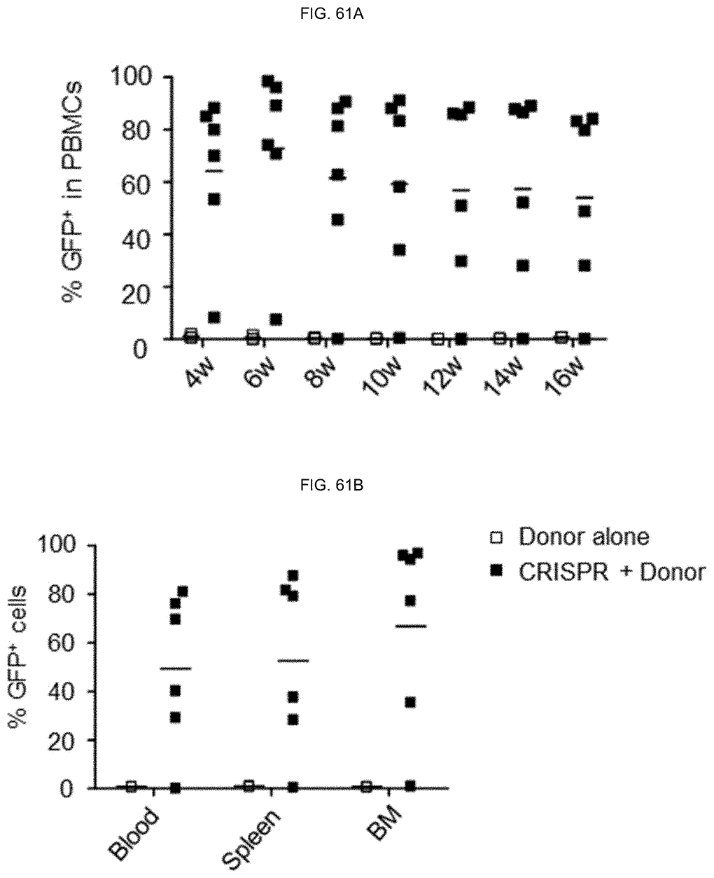

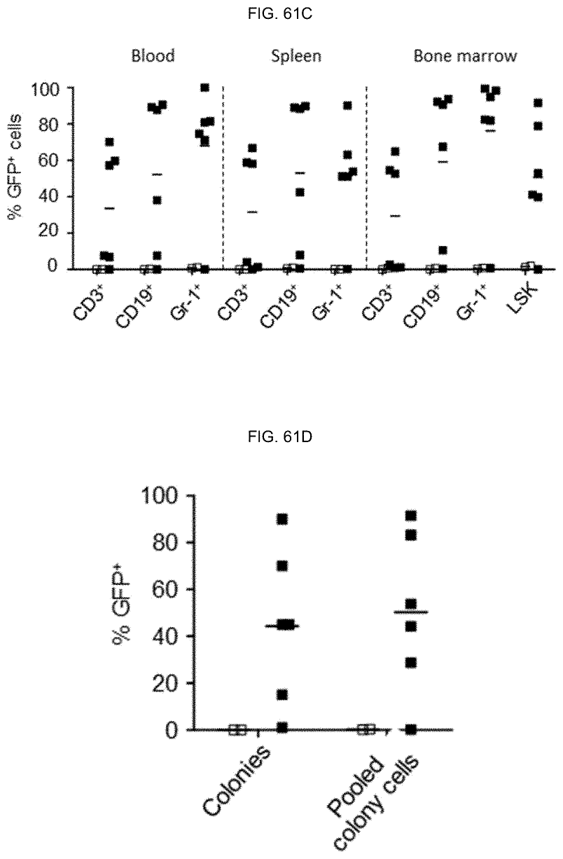

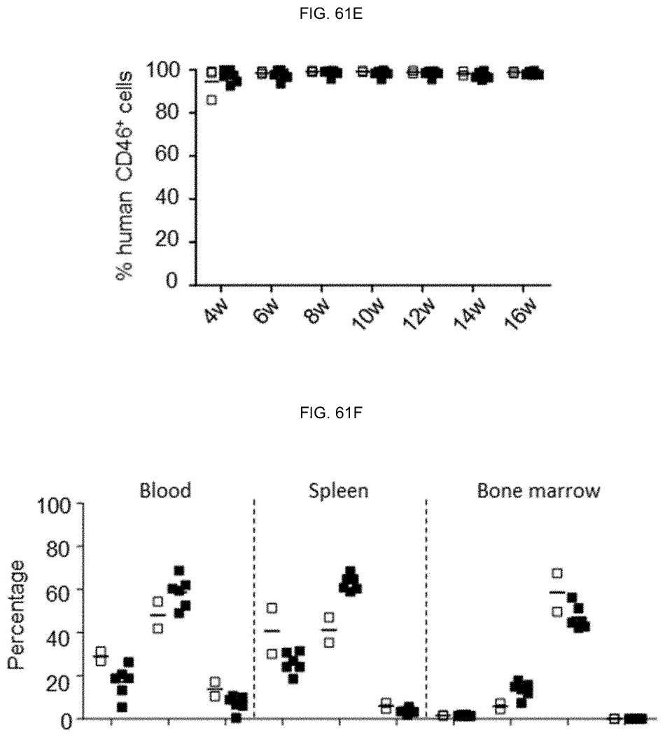

Donor cell engraftment rates were comparable for all three settings (FIG. 60) suggesting that the genomic modification introduced in HSCs by the HDAd-CRISPR and HDAd-CRISPR+HDAd-GFP-donor vector had no detrimental effects on HSC biology, specifically on the multilineage repopulation of lethally irradiated recipients. GFP marking rates reaching up to 100% appeared in PBMCs after three rounds of O⁶BG/BCNU selection of HSC/progenitors that stably expressed transgenes (FIGS. 59B, 59C). Before selection (4 weeks after transplantation), the percentage of GFP+PBMCs was 1.1%, indicating that targeted integration is a rare event. GFP+PBMCs were less than 0.2% on average in mice that were transplanted with Lin⁻ cells transduced with HDAd-GFP-donor only. This points toward the necessity of CRISPR/Cas9-mediated dsDNA breaks to achieve stable transgene expression. Mice analyzed at week 16 after transplantation showed GFP marking in all lineages analyzed in bone marrow, spleen, and PBMCs (FIG. 59D). GFP marking rates were maintained for 16 weeks in secondary transplant recipients demonstrating that primitive HSCs were genetically modified with the HDAd-CRISPR+HDAd-GFP-donor vector system (FIG. 61A), including in blood, spleen, and bone marrow (FIGS. 61B, 61C), and as shown for colonies and pooled colony cells (FIG. 61D). Percent of human CD46+ cells and percentage in blood, spleen, and bone marrow are further shown in FIGS. 61E and 61F.

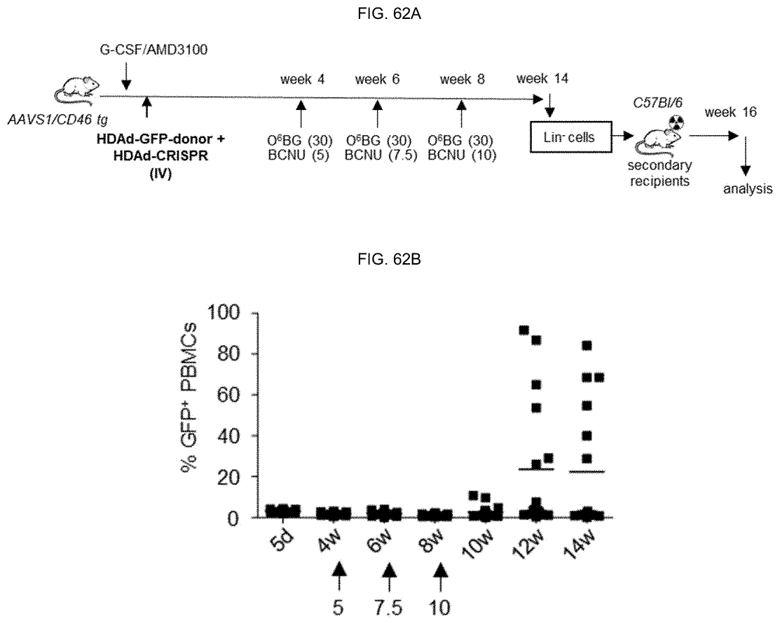

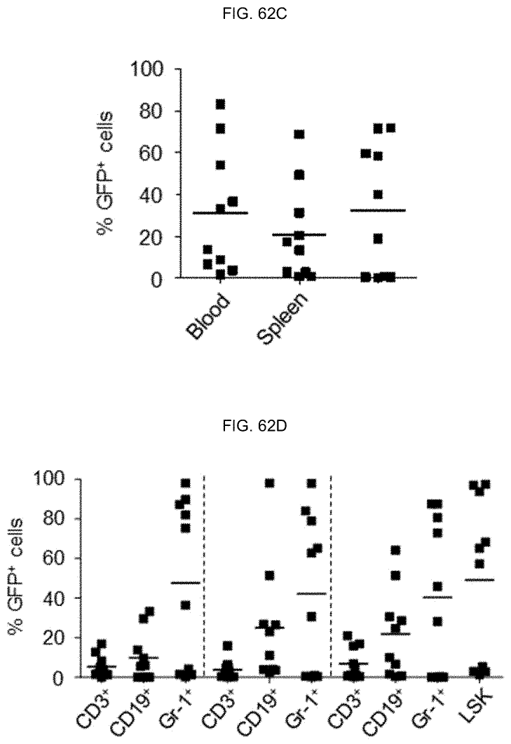

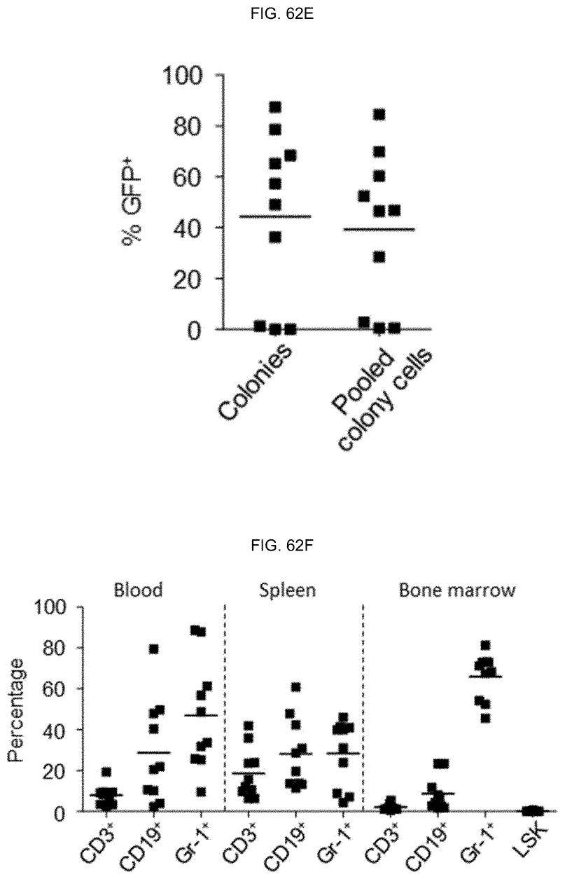

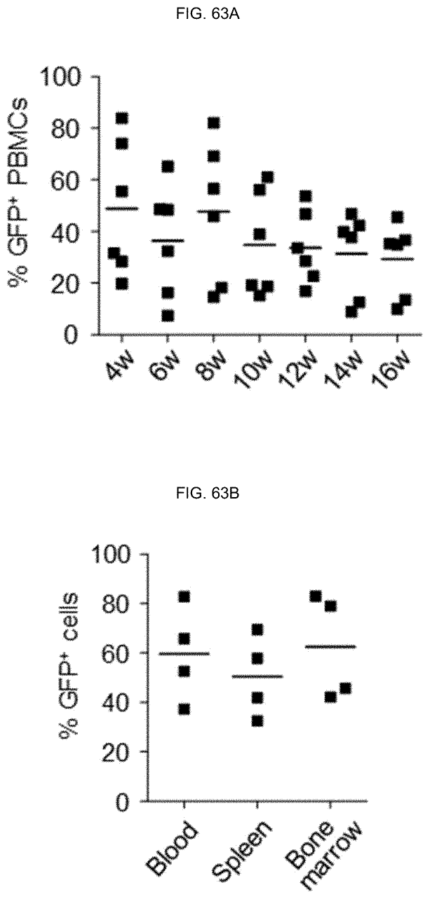

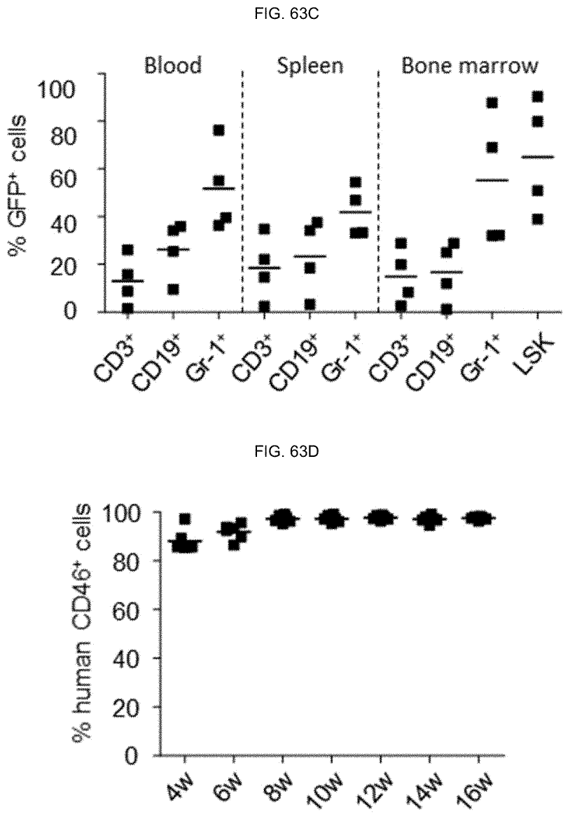

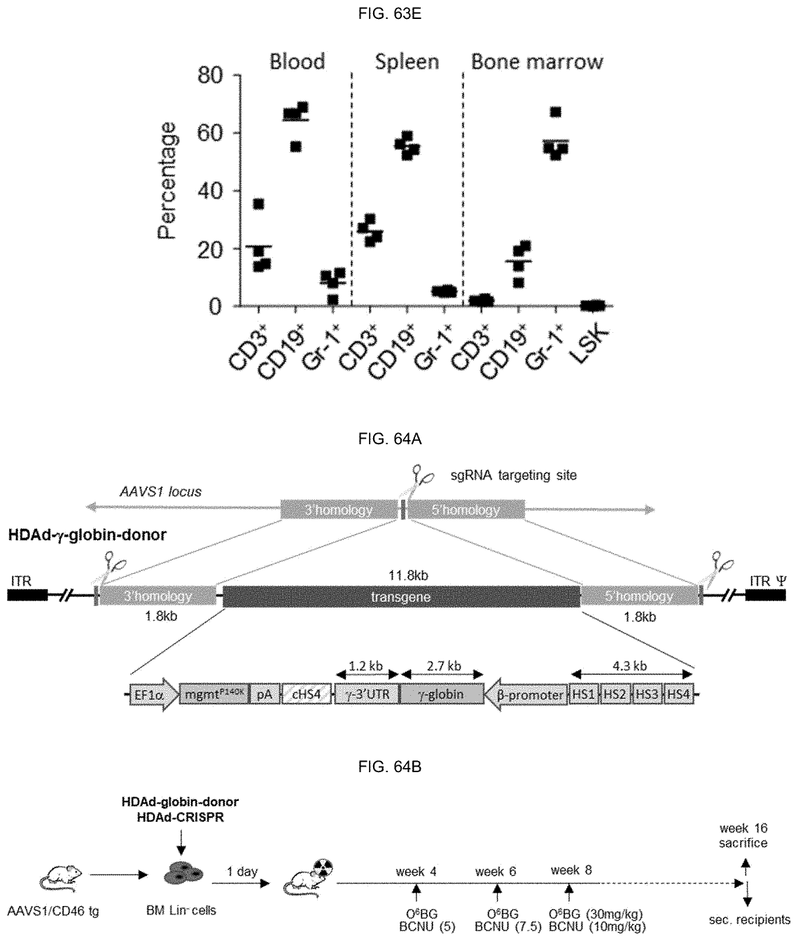

In vivo HSC transduction of AAVS1/CD46tg mice with HDAd-CRISPR+HDAd-GFP-donor. For in vivo HSC transduction of AAVS1/CD46-transgenic mice, HSCs were mobilized from the bone marrow into the peripheral blood stream by subcutaneous injections of G-CSF/AMD3100 and transduced in vivo by intravenously delivered HDAd-CRISPR+HDAd-GFP-donor vectors (FIG. 62A). After in vivo selection with three cycles of O⁶BG/BCNU, 60% of mice showed GFP expression in PBMCs ranging from 35 to 95% GFP+PBMCs in individual animals (FIG. 62B). At week 16 after in vivo transduction, similar marking was seen in mononuclear cells in blood, spleen and bone marrow (FIG. 62C). GFP marking was seen in CD3+, CD19+, and Gr-1+lineage cells in the blood, spleen and bone marrow (FIG. 62D). In the bone marrow of “responders”, more than 50% of LSK cells (a fraction that is enriched for HSCs) were GFP-positive (FIG. 62D, last group). This was also reflected by a functional assay for HSCs, the ability to form progenitor colonies (FIG. 62E). Furthermore, the transduction of primitive, long-term repopulating HSCs was shown in secondary recipients (see percentage of GFP+PBMCs at indicated time points (FIG. 63A), percentage of GFP+ cells in blood, spleen, and bone marrow (FIGS. 63B, 63C); percentage of human CD46+ cells (FIG. 63D), and percentage in blood, spleen, and bone marrow (FIG. 63E)). The in vivo HSC transduction/selection procedure had no negative influence on bone marrow cell composition and hematopoiesis (FIG. 62F).

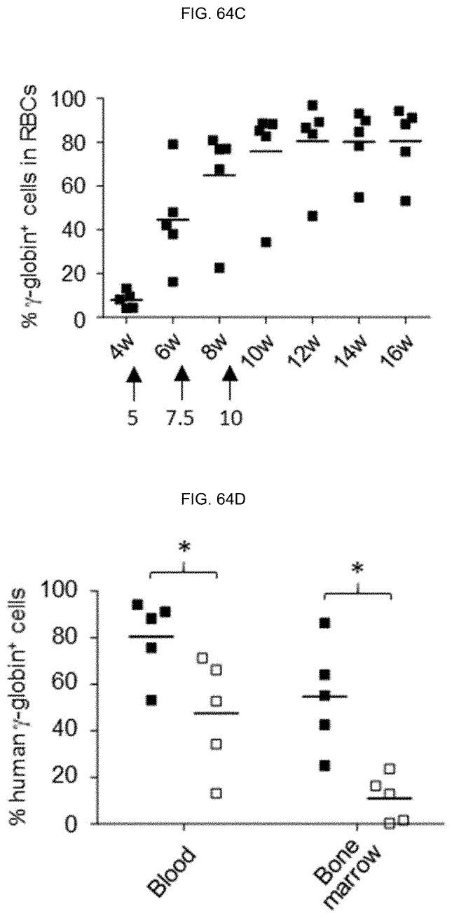

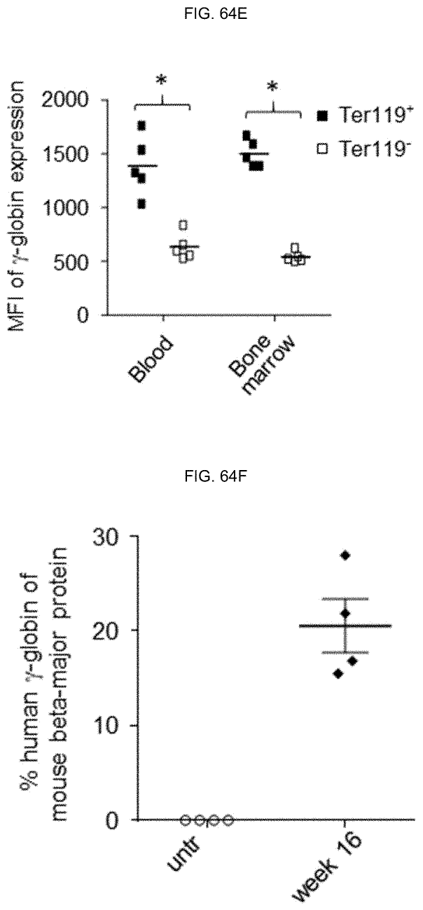

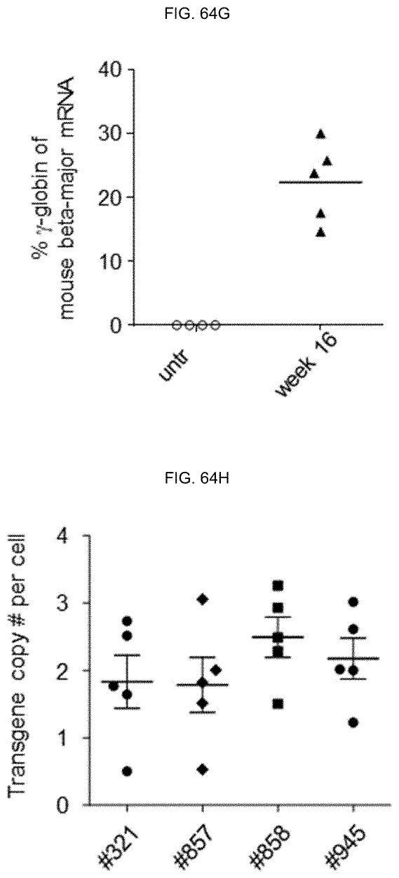

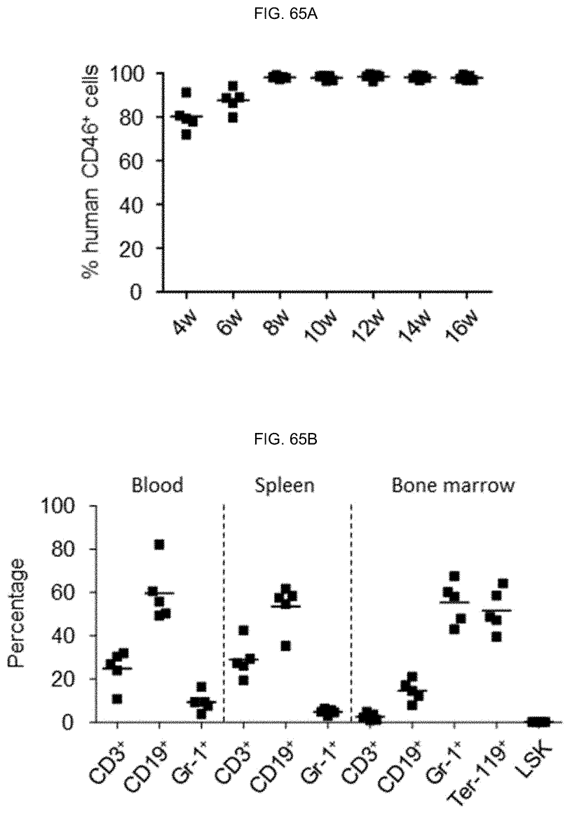

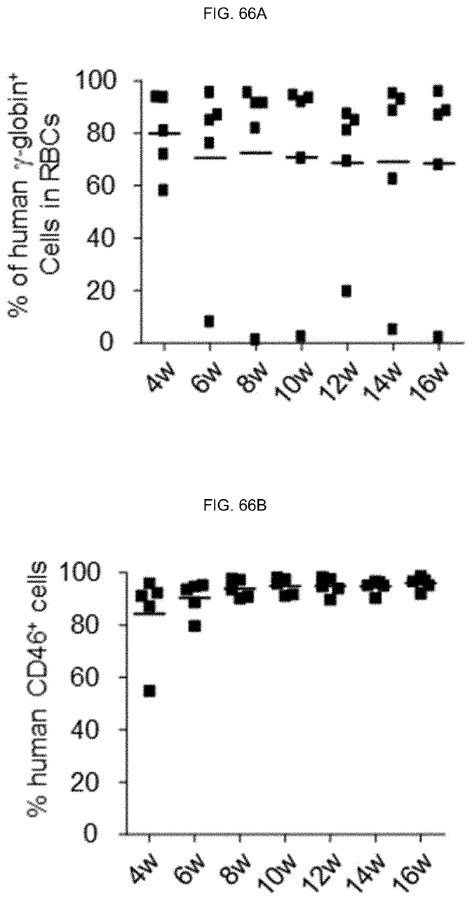

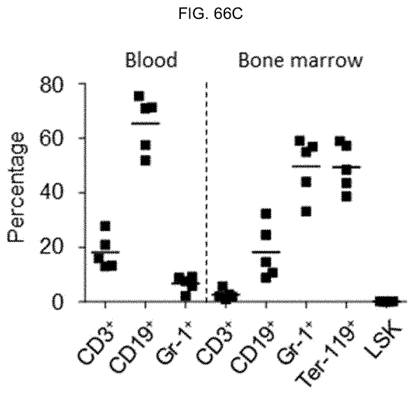

Ex vivo and in vivo HSC transduction the HDAd-CRISPR and HDAd-globin-donor vector. While the studies with the HDAd-GFP-donor vector suggest stable HSC transduction in the majority of animals, a higher rate of responders would be desirable. This would require increasing the efficacy of HDR-mediated integration, which can be achieved by increasing the length of the homology arms (Balamotis et al., Virology, 324, 229-237, 2004, Ohbayashi et al., Proc Natl Acad Sci USA, 102, 13628-13633, 2005, Suzuki et al., Proc Natl Acad Sci USA, 105, 13781-13786, 2008). A new HDAd-donor vector with 1.8 kb regions that were homologous to AAVS1 genomic sequences surrounding the CRISPR/Cas9 cleavage site were generated (FIG. 64A). For application in gene therapy of hemoglobinopathies, the human γ-globin gene (HBG1) under control of a mini γ-globin LCR was used. The HDAd-globin-donor vector were both tested in the ex vivo and in vivo HSC transduction protocols. In the ex vivo transduction setting (FIG. 64B), it was observed that all mice responded mice expressing γ-globin in 80% of peripheral red blood cells (RBCs) (FIG. 64C). The percentage of γ-globin-positive erythroid (Ter119+) cells in the blood and bone marrow was significantly higher than that of non-erythroid (Ter119⁻) cells (FIG. 64D). The same was the case for the γ-globin MFI (FIG. 64E). This suggests that the mini-LCR confers preferential expression in erythroid cells. At week 16, the level of γ-globin was 20.52(+/−5.66)% of that of adult mouse γ-globin measured by HPLC (FIG. 64F) and 22.33(+/−6.21)% by qRT-PCR (FIG. 64G). In a previous study, performed under the same regimen with the SB100x-system, γ-globin expression levels were 15.74(+/−2.69)% by HPLC and 15.40(+/−9.21)% by qRT-PCR (Li et al., Mol Ther Methods Clin Dev, 9, 142-152, 2018). This implies that the level of γ-globin expression is higher for the targeted integration system compared to the SB100x system. In fact, for the targeted integration system it would be in the range of curative levels, which is thought to be 20% γ-globin of adult globin for patients with β₀/β₀ thalassemia or sickle cell disease (Wang et al., J Clin Invest, 129, 598-615, 2019). In agreement with previous studies (Wang et al., J Clin Invest, 129, 598-615, 2019), two integrated vector copies per genome at week 16 were measured in single Lin⁻ cell-derived colonies on average (FIG. 64H). Ex vivo HSC transduction of Lin⁻ cells did not affect their ability for multilineage engraftment and complete hematopoietic reconstitution in lethally irradiated recipients (see percentage of human CD46+ cells at indicated time points (FIG. 65A), percentage in blood, spleen, and bone marrow (FIG. 65B)). Analysis of secondary HSC transplant recipients showed that ex vivo transduction with the HDAd-CRISPR+HDAd-globin-donor vector followed by in vivo selection did not affect the pool of HSCs capable of long-term repopulation (see percentage of human γ-globin⁺ cells in RBCs (FIG. 66A), percentage of human CD46+ cells (FIG. 66B), and percentage in blood and bone marrow (FIG. 66C)).

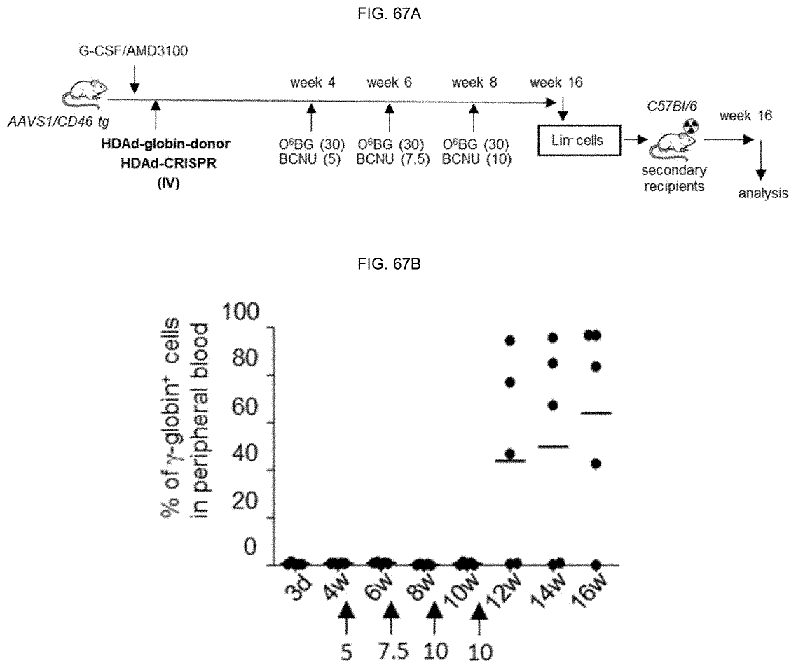

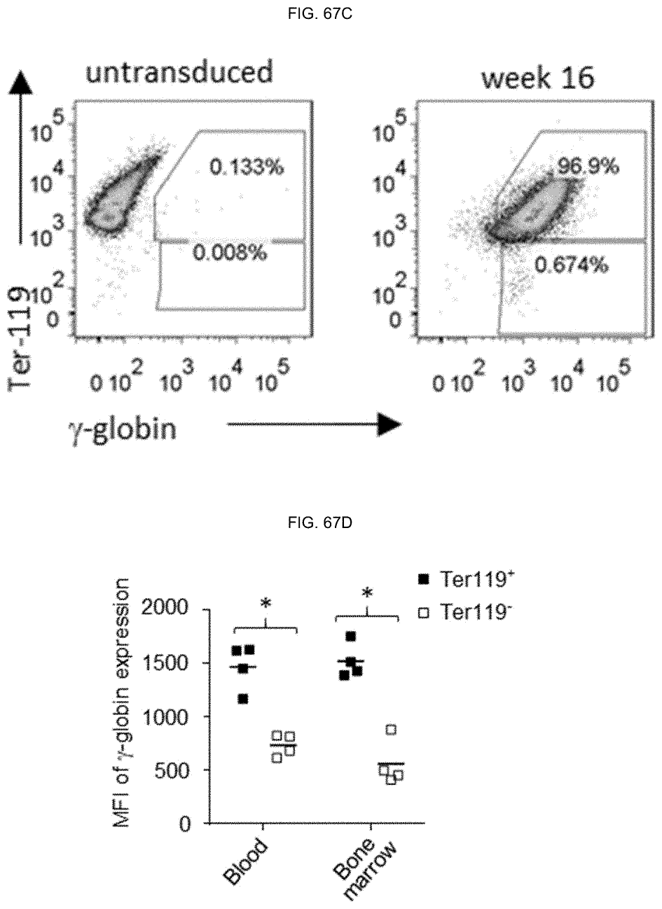

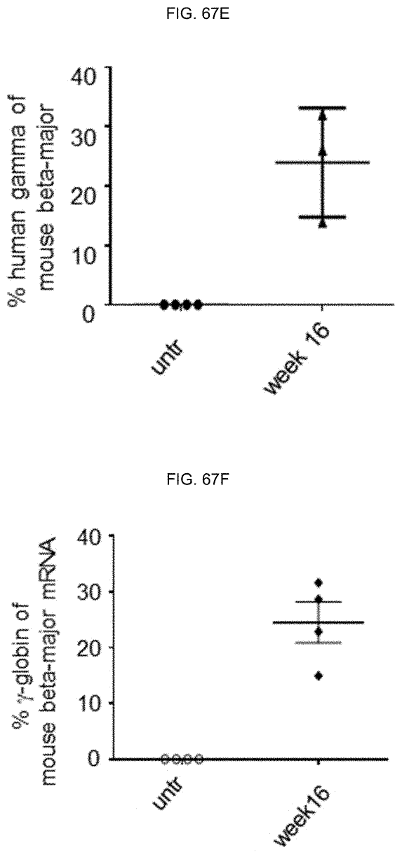

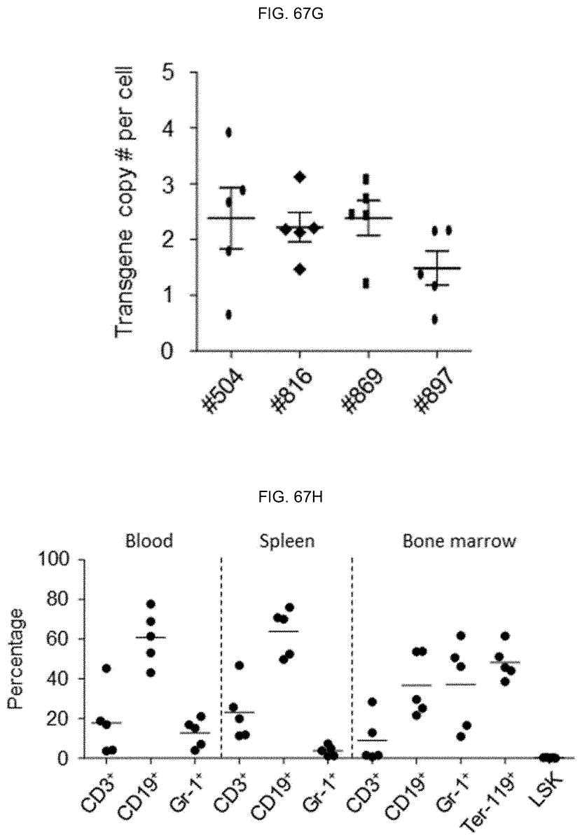

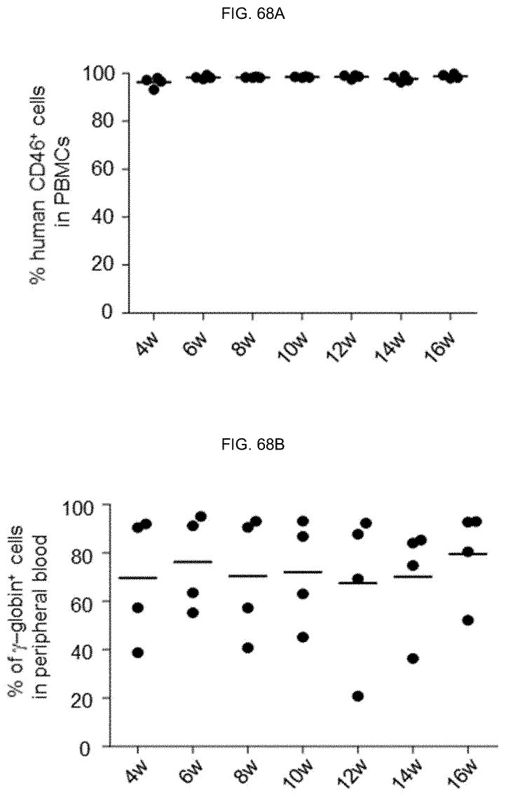

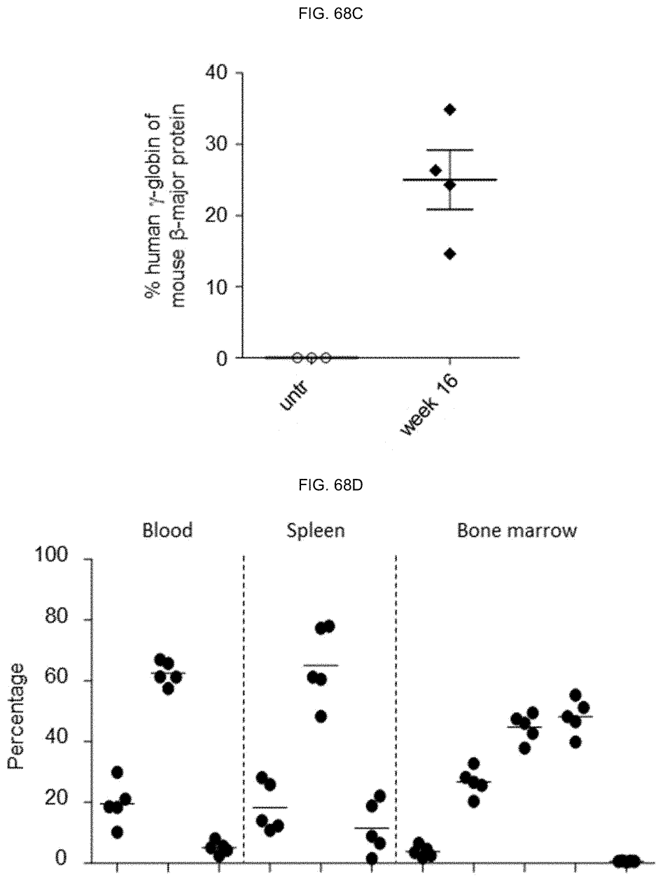

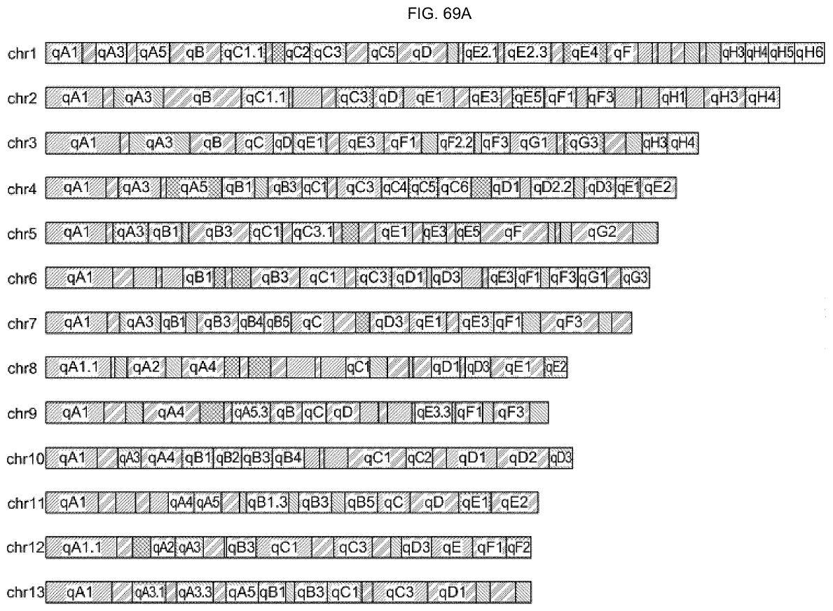

In the in vivo HSC transduction studies with the HDAd-CRISPR+HDAd-globin-donor vector (FIG. 67A), after in vivo selection, 4 out of 5 mice showed stable γ-globin expression in RBCs, ranging from 40 to 97% γ-globin⁺ RBCs in individual mice (FIG. 67B). γ-globin expression was found preferentially in erythroid cells (FIGS. 67C, 67D). The γ-globin expression levels in RBCs were 23.97(+/−7.22)% by HPLC (FIGS. 67E, 67H) and 24.53(+/−7.34)% by qRT-PCR (FIG. 67F) of that of adult mouse γ-globin. The vector copy number per cell ranged from 1.5 to 2.5 in individual mice (FIG. 67G). In the same in vivo HSC transduction/selection setting, using the SB100x based γ-globin vectors, γ-globin levels were 10.5(+/−3.1)% by HPLC and 12.17(+/−3.38)% by qRT-PCR with an average of 2 integrated vector copies per genome (Wang et al., J Clin Invest, 129, 598-615, 2019). Transplantation of bone marrow Lin⁻ cells harvested at week 16 after in vivo transduction with HDAd-CRISPR+HDAd-globin-donor into lethally irradiated recipients showed 100% engraftment and stable γ-globin expression in RBCs over 16 weeks with an average level of 24% γ- of adult β-globin (see percentage of human CD46+ cells in PBMCs at indicated time points (FIG. 68A); percentage of γ-globin⁺ cells in peripheral blood at indicated time points (FIG. 68B); human γ-globin as a percentage of mouse β-major protein (FIG. 68C); and percentage in blood, spleen, and bone marrow (FIG. 68D)).

In summary, the HSC transduction studies with HDAd-CRISPR+HDAd-globin-donor resulted in stable γ-globin expression at levels that are significantly higher than those achieved in previous studies with the SB100x-based system.

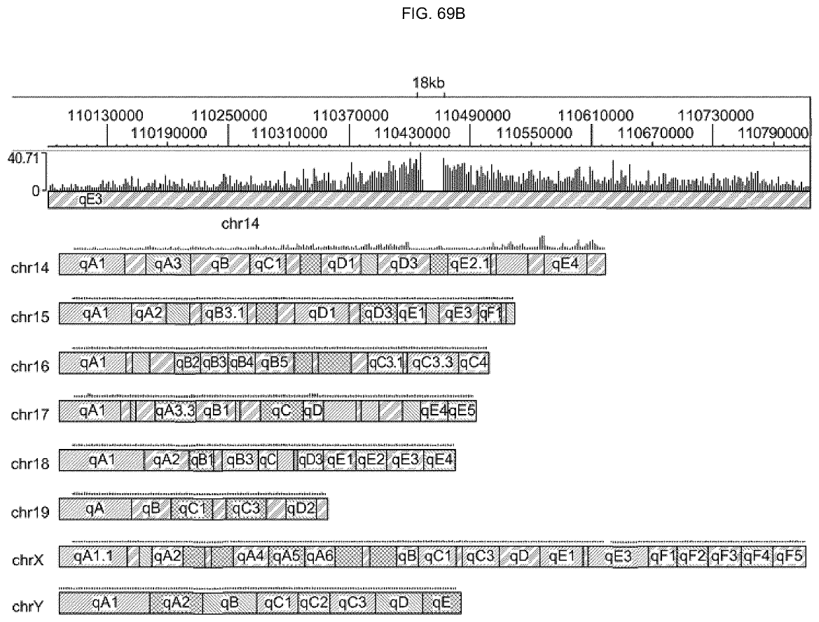

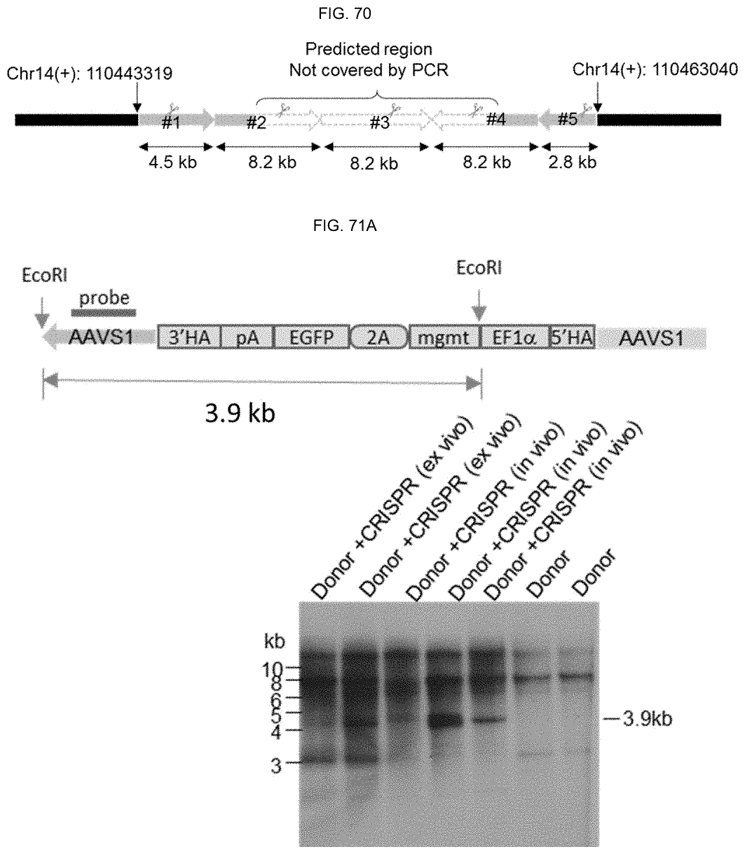

Localization of the AAVS1 locus in AAVS transgenic mice. Inverse PCR (iPCR) for integration site analysis requires the knowledge of the AAVS1 locus localization in the genome of AAVS1/CD46-transgenic mice. To determine this, a targeted locus amplification (TLA)/PCR technology that involves the crosslinking of physically proximal sequences was used (de Vree et al., Nat Biotechnol, 32, 1019-1025 2014; see Material and Methods). The TLA data obtained from bone marrow cells from AAVS1/CD46-tg mice were then aligned with a reference mouse genome (FIG. 69). TLA results indicate that the 18 kb AAVS1 locus is integrated into chromosome 14 at the location (Chr14:110443871-110461834) (FIG. 55B). Using this information, primers were used to sequence into the locus (FIG. 70). Repeats of the AAVS1 locus facing left-to-right and right-to-left were found. Both terminal repeats (#1 and #5) were truncated and 4.5 and 2.8 kb long, respectively. Repeat #5 lacked a complete 5′ homology region. This constellation of target sites complicated the integration site analysis. Some of the theoretical outcomes for the integration by the HDAd-CRISPR+HDAd-donor system the outlined in FIG. 70.

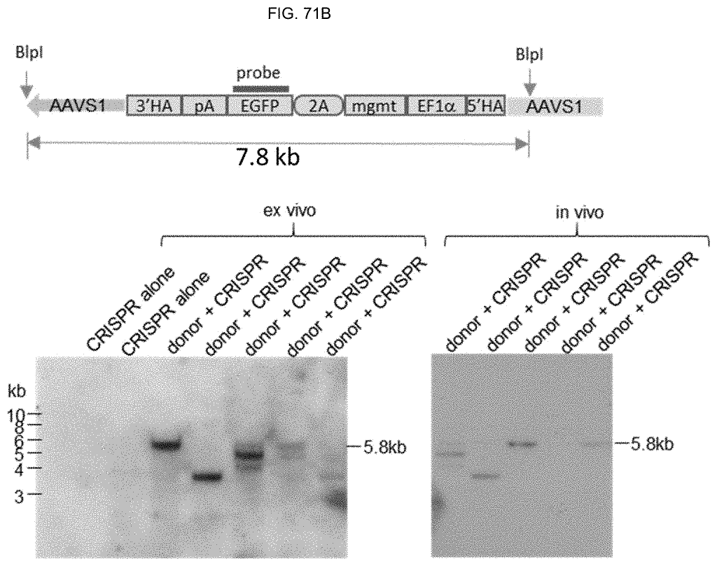

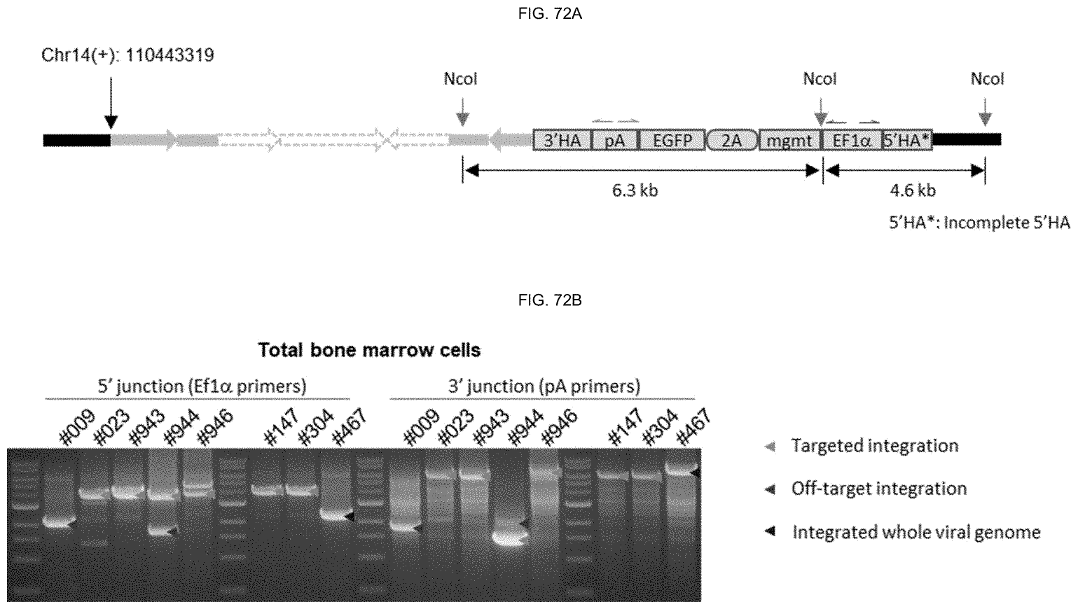

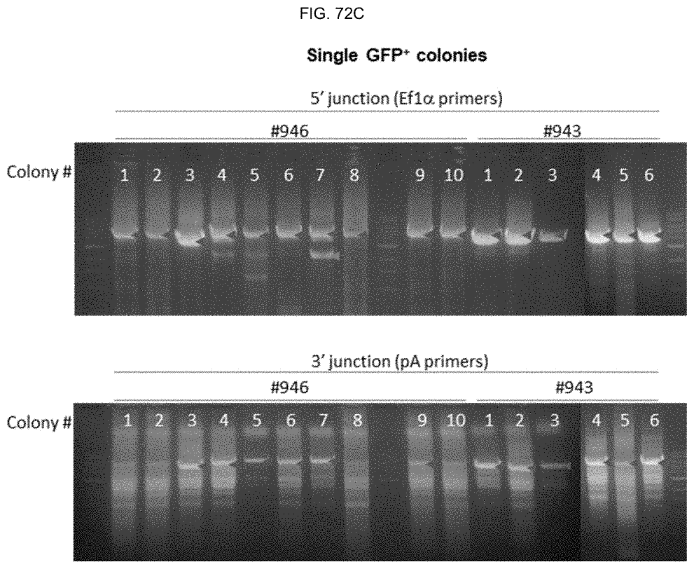

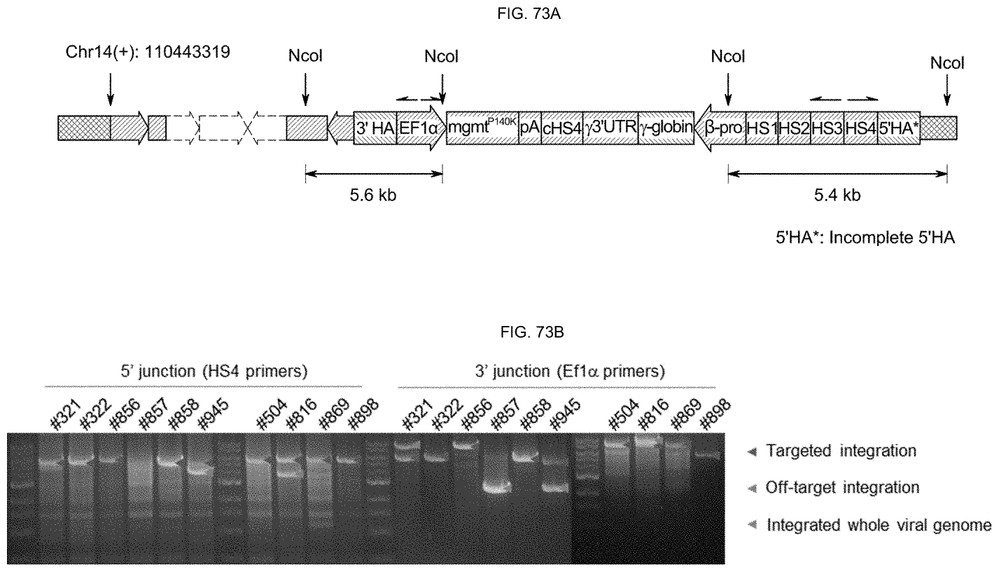

Chromosomal integration after ex vivo and in vivo HSC transduction with HDAd-CRISPR+HDAd-donor. First genomic Southern blot was performed on DNA from bone marrow cells harvested at week 16. Hybridization of EcoRI-digested genomic DNA with an AAVS1 specific probe showed in all analyzed mice a 3.9 kb-specific band indicative for integration of the donor cassette into one (or more) repeats of the AAVS1 locus (FIG. 71A). Hybridization of Blp1-digested DNA with the GFP probe resulted in 5.8 kb signals in 5 out 10 mice representative for integration into the full-length repeats #2-4 (FIG. 71B). The 5 and 6 kb signals could be the result of integration into repeat #1 and 5, respectively. Two out of ten mice appeared to have integrations into several AAVS1 motif repeats. To demonstrate the presence of transgene/chromosome junctions, iPCR was performed on genomic DNA from mice (FIGS. 72A, 72B). Six out of eight mice analyzed displayed PCR products consistent with HDR-mediated integration into the AAVS1 site (FIG. 72B). Several of these mice had additional bands that resulted from integration into one of the CRISPR/Cas9 off-target sites on chromosome 5 (FIG. 72B). Bands that originated from integration of the full-length HDAd genome involving the ITRs as junctions were also found. Interestingly, these integrated full-length HDAd genomes were on chromosome 14, the chromosome containing the CRISPR AAVS1 target site (FIG. 72B). In an attempt to de-complex these results derived from a pool of bone marrow cells, d GFP+bone marrow Lin⁻ cells were plated to generate progenitor colonies derived from single cells (FIG. 72C). Analysis of colonies from mice with only one band specific for HDR-integration into AAVS1 (e.g. mouse #943) showed homogenous signals in all colonies, whereas colonies from mice with additional off-target integration (e.g. #946) showed a chimeric pattern: nine out of ten colonies with only on-target integration, one colony containing both on-target and off-target integrations, which is possible because the average number of integrated transgenes per genome is 2. Integration site analysis of bone marrow cells in the ex vivo and in vivo transduction studies with HDAd-CRISPR and HDAd-globin-donor vector revealed a similar outcome (FIGS. 73A & 73B, showing on-target integration (FIG. 73A) and samples with on- and/or off-target integration (FIG. 73B)). In the ex vivo HSC transduction setting with HDAd-CRISPR +HDAd-globin-donor, a higher rate of animals with targeted integrations were found compared to the in vivo HSC transduction study with the HDAd-GFP-donor vector. This may be due to a higher HDR efficacy based on longer homology regions.

Overall these integration studies indicate a high frequency of targeted integrations into the AAVS1 loci. A fraction of integrations occurred into CRISPR off-target sites and possibly into regions that involved CRISPR-triggered large deletions on the chromosome that contained the target site.

Discussion Self-inactivating lentivirus vectors, in contrast to gamma-retrovirus vectors, have not been associated with insertion site-associated malignant clonal expansions in clinical HSC gene therapy trials. However, this risk cannot be completely excluded as a recent study in non-human primates indicates (Espinoza et al., Mol Ther, 6, 1074-1086, 2019). Theoretically, the random integration pattern mediated by SB100x and the lack of a preference for integration into activate genes and promoters should be safer but concerns about genotoxicity remain. Therefore, a major effort in the field is aimed toward targeted transgene integration into preselected sites such as the AAVS1 site. Zinc finger nuclease mRNA and AAV6-mediated donor template delivery in human HSCs resulted in >50% targeted integration into the AAVS1 locus (De Ravin et al. Nat Biotechnol, 34, 424-429, 2016). In other studies that employed an AAVS1-specific CRISPR/Cas9 RNP and AAV6 to deliver the donor template, the frequency of site-specific integration was 25% (Johnson et al., 2018. Sci Rep, 8, 12144). Similar rates were achieved for targeted integration into CCR5 (Hung et al., Mol Ther, 26, 456-467, 2018).

This approach for targeted integration into AAVS1 has a number of new aspects. (i) The use of a helper-dependent, capsid-modified HDAdvector to deliver the donor template. Corresponding genomes are double stranded, linear DNA covalently linked on both ends to the viral TP protein. It is thought that, in contrast to single-stranded AAV6 donor vectors, double-stranded, linear adenoviral DNA is not an optimal template for HDR. To compensate for this potential disadvantage, AAVS1 CRISPR/Cas9 cleavage sites were incorporated into the HDAd-donor vectors to create free “recombinogenic” DNA ends. (ii) Because the insert capacity of HDAdvectors is 30 kb it was possible to incorporate homology arms that would exceed the packaging capacity of rAAV6 or IDLV vectors. Previous studies (Balamotis et al., Virology, 324, 29-237, 2004, Ohbayashi et al., Proc Natl Acad Sci USA, 102, 13628-13633, 2005, and Suzuki et al., Proc Natl Acad Sci USA, 105, 13781-13786, 2008) and the comparison of HDAd-donor vectors with 0.8 and 1.8 kb homology regions suggest that increasing the homology improved the number of responder mice with high level transgene expression as well as the fraction of mice with targeted integration. (iii) The large HDAd5/35++insert capacity also allowed for the inclusion of the mgmt^{P140K}-based in vivo selection cassette into the donor template, thus mediating selective survival and expansion of progeny cells without affecting the pool of transduced primitive HSCs by short term treatment with low-dose O⁶BG/BCNU (Wang et al., Mol Ther Methods Clin Dev, 8, 52-64, 2018). Considering the low efficacy of HDR and consequently targeted integration in HSC (Genovese et al., Nature, 510, 235-240, 2014), in vivo HSC selection appears to be crucial to achieve high transgene marking levels in peripheral blood cells. (iv) Finally, because of the ease to produce high yields of HDAd5/35++ vectors and their tropism for primitive HSCs, they can be used for in vivo HSC transduction via intravenous injection into mobilized animals. Therefore, it was possible to perform a proof-of-principle of in vivo HSC gene therapy of hemoglobinopathies with targeted transgene integration.

To achieve stable transgene (GFP or γ-globin) expression, the coinfection of HDAd-donor and HDAd-CRISPR was essential, suggesting that CRISPR-mediated genomic DNA breaks and, most likely, the release of the donor template from the HDAd-donor vector greatly stimulated integration. An indicator for transgene integration into HSCs after in vivo transduction with HDAd-donor+HDAd-CRISPR was the fraction of mice that displayed stable high-level transgene expression after the completion of in vivo selection (i.e. “responders”). It was 6 out of 16 (37.5%) for the HDAd-GFP-donor+HDAd-CRISPR and 4 out of 5 (80%) for the HDAd-globin-donor+HDAd-CRISPR. Notably, the “responder” rate with a high frequency of targeted integration was 100% for both vectors in the ex vivo transduction setting. This indicates that a limiting factor for the targeted in vivo HSC transduction approach is the efficacy of HSC infection. The initial infection step could theoretically be improved by an optimized HSC mobilization regimen (Psatha et al., Hum Gene Ther Methods, 25, 317-327, 2014) and two rounds of HDAd injection one day apart.

These data indicate that the vector system is an efficient tool to achieve targeted integrations in HSC in ex vivo and in vivo transduction settings. This may be due, in large part, to the high efficacy of HDAd-donor vector delivery to the nucleus of non-dividing cells, the ability to release the donor cassette from the vector backbone, and the HDAd vectors' capacity to incorporate large homology regions.

An important finding in this study was that the targeted integration system conferred higher transgene expression levels than the SB100x-based system in the in vitro, ex vivo, and in vivo transduction setting. This is particularly relevant for gene therapy of hemoglobinopathies (β₀/β₀ thalassemia and Sickle Cell Disease) which requires γ-globin at levels that are >20% of adult globin levels. In “responder” mice that were ex vivo or in vivo transduced with the HDAd-CRISPR+HDAd-globin-donor, these theoretically curative levels were achieved. This an important improvement over a previous study in a thalassemia mouse model where the SB100x transposase system was utilized for γ-globin gene addition (Wang et al., J Clin Invest, 129, 598-615, 2019). Epigenomic effects on transgene expression may be less pronounced after integration into the AAVS1 locus which is known to maintain an open chromatin configuration in HSCs (Wang et al., Genome Res, 17, 1186-1194, 2007, Huser et al., PLoS Pathog, 6, e1000985, 2010, van Rensburg et al., Gene Ther, 20, 201-214, 2013) and in AAVS1 transgenic mice. On the other hand, it cannot be excluded that random SB100x-mediated integration places transgenes into regions that are subjected to silencing.

The integration site analyses suggest a near 100% targeted integration efficacy after in vitro transduction of HUDEP-2 cells. In ex vivo and in vivo HSC transduction studies, both Southern blot and iPCR on genomic bone marrow DNA showed efficient targeted integration in bone marrow HSCs. For example, iPCR of integration junctions documented targeted integration in 75% of mice, with most of these mice having no off-target integration. This was further confirmed by analysis of colonies derived from single CFUs. At a low frequency, integrations were also found in two of the in silico predicted CRISPR Cas9 off-target sites. Furthermore, full-length HDAd-donor genomes integrated in chromosome 14, the chromosome that carries the AAVS1 loci, were found. It was previously found that HDAd ITRs are prone to DNA breaks and that this can result in inefficient integration into genomic sites in which DNA breaks occur (Wang et al., J Virol, 79, 10999-11013, 2005, Wang et al., J Virol, 80, 11699-11709, 2006). Considering recent studies on CRISPR/Cas9-induced undesired large deletions/translocations (7-8 kb) around the target site (Kosicki et al., Nat Biotechnol, 36, 765-771, 2018), it is possible that CRISPR-Cas9 DNA breaks far away from the target site could be implicated in the integration of complete HDAd genomes. Overall, reports on large deletions/translocations question the safety of CRISPR/Cas9. On the other hand, because no developmental effects associated with CRISPR/Cas9-mediated germline editing in animals have been reported so far, it is likely that cells with such deleterious chromosomal changes are selected out during development. Support for this hypothesis comes from a recent NHP study in which CRISPR Cas9-edited HSCs were transplanted and a 9 kb deletion in the HBG1/2 region disappeared in PBMCs over time (Humbert et al., 23rd Annual Meeting of the ASGCT, abstract #974, 2019).

From these studies, it can be concluded that the AAVS1tg mouse model is suboptimal for targeted integration studies involving CRISPR/Cas9 because of the presence of multiple AAVS1 target loci, some of which were truncated to a degree that they lost areas of homology with the HDAd-donor vector. The presence of truncated AAVS1 loci also suggests that rearrangement can occur in AAVS1 transgenic mice as reported previously (Linden et Proc Natl Acad Sci USA, 93, 7966-7972, 1996).

### Example 6. Prophylactic In Vivo Hematopoietic Stem Cell Gene Therapy with an Immune Checkpoint Inhibitor Reverses Tumor Growth in a Syngeneic Mouse Tumor Model

At least some of the information contained in this example was published in Li et al. (Cancer Res. 80(3):549-560, 2020; published online Nov. 14, 2019).

Population-wide testing for cancer-associated germline mutations has established that more than one-fifth of ovarian and breast carcinomas are associated with inherited risk. Salpingo-oophorectomy and/or mastectomy are currently the only effective options offered to women with high-risk mutations. The goal is to develop a long-lasting approach that provides immuno-prophylaxis for carriers of inherited mutations. This approach leverages the fact that at early stages, tumors recruit hematopoietic stem/progenitor cells (HSPCs) from bone marrow and differentiate them into tumor-promoting cells. A technically simple technology has been developed to genetically modify HSPCs in vivo. The technology involves HSPC mobilization and intravenous injection of an integrating HDAd5/35++ vector. In vivo HSPC transduction with a GFP-expressing vector and subsequent implantation of syngeneic tumor cells showed >80% GFP-marking in tumor infiltrating leukocytes. To control expression of transgenes, a miRNA regulation system that is activated only when HSPCs are recruited to and differentiated by the tumor was developed. The approach was tested using the immune checkpoint inhibitor αPDD-L1-γ₁ as an effector gene. In in vivo HSPC-transduced mice with implanted mouse mammary carcinoma (MMC) tumors, after initial tumor growth, tumors regressed and did not recur throughout the observation period. The regression was T-cell mediated. “Conventional” treatment with an anti-PD-L1 monoclonal antibody had no significant anti-tumor effect, indicating that early, self-activating expression of αPDD-L1-γ₁ can overcome the immunosuppressive environment in MMC tumors. The efficacy and safety of the approach was further validated in an ovarian cancer model with typical germ-line mutations (ID8 p53^{-/-} brca2^{-/-}), both in a prophylactic and therapeutic setting.

Materials and Methods.

HDAd5/35++ vectors: HDAd-SB is described in Richter et al., Blood. 128: 2206-2217, 2016. The mouse αPD-L1-γ1 transgene is described in Engeland et al., Mol Ther. 22: 1949-1959, 2014); and the production of HDAd5/35++ vectors in 116 cells is described in Palmer et al., Methods in Molecular Biology, 33-53, 2009. Helper virus contamination levels were found to be <0.05%. Titers were 6-12×10¹² vp/ml. All HDAd vectors used in this study contain chimeric fibers composed of the Ad5 fiber tail, the Ad35 fiber shaft, and the affinity-enhanced Ad35++fiber knob (Wang et al., J Virol. 82: 10567-10579, 2008). All of the HDAd preparations had less than one copy wild-type virus in 1010 vp measured by qPCR using the primers described elsewhere (Haussler et al., PLoS One. 6: e23160, 2011)

Construction of the HDAd-GFP/mgmt and HDAd-αPD-L1γ₁miR423 vectors. Step 1: The PGK promoter, β-globin 3′ UTR and BGH polyA fragments were PCR amplified from pHCA-HBG-CRISPR/mgmt. (Li et al., Blood. 2018; 131: 2915-2928), followed by insertion into the BstBI site of pBS-Z-Ef1α (Saydaminova et al., Mol Ther Methods Clin Dev. 1: 14057, 2015) by Gibson assembly (New England Biolabs), generating pBS-PGK-3′UTR. The GFP coding sequence was PCR amplified from pHM5-frt-IR-EF1α-mgmt-2a-GFP (Wang et al., Mol Ther Methods Clin Dev. 8: 52-64, 2018) and ligated with EcoRI linearized pBS-PGK-3′UTR, generating pBS-PGK-GFP. Step 2: The Ef1α-mgmt^{P140K}-SV40 pA-cHS4 insulator cassette was amplified from pHM5-T/pLCR-γ-globin-mgmt-FRT2 (Li et al., Mol Ther Methods Clin Dev 9: 142-152, 2018) and ligated with PacI-digested pHM5-T/pLCR-γ-globin-mgmt-FRT2, forming pHM5-FRT-IR-Ef1α-mgmt. A BsrGI site at 3′ side of cHS4 was introduced by primer for downstream use. The bacterium plasmid backbone of pHM5-FRT-IR-Ef1α-mgmt was switched to the backbone from pBS-Z-Ef1a using primers containing 15 bp homology arm (HA) for later infusion cloning (Takara, Mountain View, Calif.), generating pBS-FRT-IR-Ef1α-mgmt. The two 15 bp HAs flanking the two Frt-IR components can be exposed upon PacI digestion to facilitate recombination with the modified pHCA construct described below. Then, the PGK-GFP-3′UTR-BGHpA fragment was moved from pBS-PGK-GFP in step 1 to the BsrGI site of pBS-FRT-IR-Ef1α-mgmt, generating pBS-FRT-IR-GFP/mgmt. Step 3: The original PacI site in pHCA was destroyed by inserting two annealed oligo sequences. A new PacI site together with two HAs were created at BstBI site. Finally, after PacI digestion of both pBS-FRT-IR-GFP/mgmt and modified pHCA, the products were recombined by infusion cloning, generating pHCA-FRT-IR-GFP/mgmt, which was used for subsequent virus rescue. HDAd-αPDD-L1γ₁ was constructed similarly as HDAd-GFP/mgmt described elsewhere in this Example, except instead of GFP coding sequence, the anti-PD-L1-γ₁ transgene was inserted into the EcoRI of pBS-PGK-3′UTR at step 1. For microRNA regulated gene expression, synthesized 4×miR423 oligos (forward (SEQ ID NO: 24); reverse (SEQ ID NO: 25)) were annealed and inserted into the AvrII-XhoI sites of pBS-PGK-3′UTR, generating pBS-PGK-miR423-3′UTR, which was then used for anti-PD-L1-γ₁ insertion.

HDAd-GFP-423 was constructed in a similar way by inserting the 4×miR423 target sites into the 3′UTR of HDAd-GFP/mgmt.

Flow cytometry: Cells were resuspended at 1×10⁶ cells/100 μL in PBS supplemented with 1% FCS and incubated with FcR blocking reagent (Miltenyi Biotech, Auburn Calif.) for ten minutes on ice. Next the staining antibody solution was added at 100 μL per 10⁶ cells and incubated on ice for 30 minutes in the dark. After incubation, cells were washed once in FACS buffer (PBS, 1% FBS). For secondary staining the staining step was repeated with a secondary staining solution. After the wash, cells were resuspended in FACS buffer and analyzed using a LSRII flow cytometer (BD Biosciences, San Jose, Calif.). Debris was excluded using a forward scatter-area and sideward scatter-area gate. Single cells were then gated using a forward scatter-height and forward scatter-width gate. Flow cytometry data were then analyzed using FlowJo (version 10.0.8, FlowJo, LLC). Matched isotype-controls were included in all experiments.

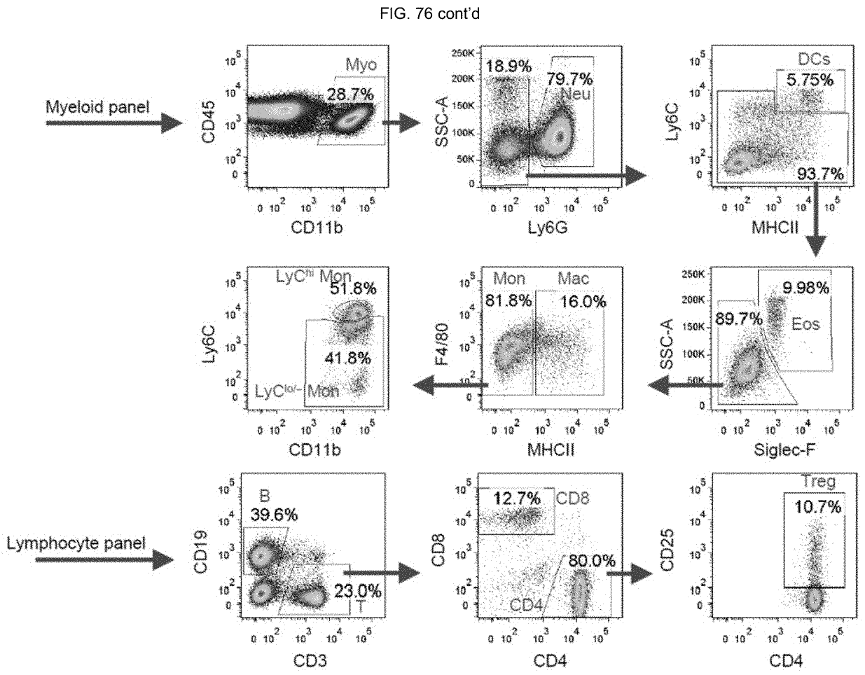

Flow cytometry for immunophenotyping: Lymphocyte flow cytometry panel 8c (CD45-APC/Cy7, clone 30-F11, cat #103116; CD3-APC, clone 17A2, cat #100236; CD4-PE/Cy7, clone GK1.5, cat #100422; CD8a-PE, clone 53-6.7, cat #100708; CD25-BV421, clone PC61, cat #102043; CD19-6V510, clone 6D5, cat #115546; all these antibodies were from BioLegend) and myeloid panel 9c (CD45-APC/Cy7, clone 30-F11, BioLegend, cat #103116; CD11c-APC, clone N418, BioLegend, cat #117310; F4/80-PE, clone C1:A3-1, Cedarlane, cat #CL8940PE; MHCII-BV510, clone M5/114.15.2, BioLegend, cat #107635; Siglec F-PerCP, clone 1RNM44N, eBioscience, cat #46-1702-82; Ly6C-BV421, clone AL-21, BD Biosciences, cat #562727; CD11b-PE/Cy7, clone M1/70, eBioscience, cat #25-0112-82; Ly6G-BV605, clone 1A8, BioLegend, cat #127639) were used. The gating strategy is shown in FIG. 76. LSK (lineage⁻/Sca-1⁺/c-Kit⁺) cells were characterized previously in Richter et al., Blood. 2016; 128: 2206-2217. The following antibodies were also used: biotin-conjugated lineage detection cocktail (Miltenyi Biotec, San Diego, cat #130-092-613); anti-mouse LY-6A/E (Sca-1)-PE-Cyanine7 (clone D7, eBioscience, San Diego, cat #25-5981-82); anti-mouse CD117 (c-Kit)-PE (Clone 2B8, eBioscience, San Diego, cat #12-1171-83); anti-mouse CD3-APC (clone 17A2, Invitrogen, Waltham, Mass., cat #17-0032-82); anti-mouse CD19-PE-Cyanine7 (clone eBio1D3, eBioscience, San Diego, cat #25-0193-82); anti-mouse Ly-6G (Gr-1)-PE, (clone RB6-8C5, eBioscience, San Diego, Calif., cat #12-5931-82); anti-human CD46-APC (clone E4.3, BD Pharmingen, San Diego, Calif., cat #564253).

IFNγ-flow cytometry: Splenocytes were isolated by passing freshly harvested spleen through a 70 μm cell strainer attached to a 50 mL Falcon tube. After centrifugation at 300×g for 10 minutes, red blood cells were removed by resuspending cells in 1 mL 1×BD Pharm Lyse™ lysing solution (BD Pharmingen, San Diego, Calif., cat #555899) and incubating for 30 seconds. 20 mL RPMI-1640 medium was added to stop lysing reaction. Following centrifugation and resuspension in RPMI-1640 medium with 10% heat-inactivated FBS, 100 units/ml penicillin and 100 mg/ml streptomycin, the obtained splenocytes were cultured at 5×10⁶ cells/ml (200 μl/well) in 96-well tissue culture plates in a humidified incubator with 5% CO₂. 1× Cell Stimulation Cocktail plus protein transport inhibitors (eBioscience, San Diego, cat #00-4975-93) was presented in the culture medium for induction and accumulation of IFN-γ production within the cells. After stimulation for 12 hours, the cells were collected, stained first with cell surface markers as described above, and then subject to intracellular staining for IFN-γ (BioLegend, San Diego, Calif., cat #505842) according to the manufacturer's instructions.

Neu-tetramer flow cytometry: The PE-labeled H-2Dq/RNEU420-429 (H-2D(q) PDSLRDLSVF) (SEQ ID NO: 290) tetramer was obtained from the National Institute of Allergy and Infectious Diseases MHC Tetramer Core Facility (Atlanta, Ga.), and used according to the manufacturer's instructions.

Isolation of tumor-infiltrating leukocytes for flow cytometry, FACS, and Western blot: Mice were sacrificed when tumor volume reached 500 mm³. Tumors were harvested, diced and digested with 300 U/mL Collagenase I (Sigma-Aldrich, St. Louis, Mo., cat #C0130) and 1 mg/mL Dispase II (Sigma-Aldrich, cat #4942078001) in 5 mL of RPMI 1640 for 30 minutes at 37° C. with gentle mixing. After digestion, 2000 U/mL DNase I (Sigma-Aldrich, cat #260913) was added to reduce viscosity by removing released DNA. Single cell suspension was obtained by passing the digested tissue through a 70 μm cell strainer using a syringe plunger. Subsequently, tumor infiltrating leukocytes were purified from the single cell suspension using mouse CD45 (TIL) MicroBeads (Miltenyi Biotech, Auburn Calif., cat #130-110-618).

Immunofluorescence studies: Tumor slides were fixed with acetone/methanol (10 min) and washed twice with PBS. Slides were blocked for 20 min at room temperature using PBS with 5% blotting grade milk (Bio-Rad, Hercules, Calif.) followed by incubation with primary antibodies in PBS for 1 h at room temperature. Then slides were washed twice with PBS and incubated with secondary antibodies for 1 h at room temperature followed by washing with PBS three times. Slides were washed twice with PBS, mounted with Mounting Medium for Fluorescence (Vector Laboratories Burlingame, Calif.) and then analyzed using a fluorescence microscope. Laminin was detected using anti-laminin polyclonal (primary) antibody (1:200; #Z0097; Dako, Carpinteria, Calif.) and goat anti-rabbit IgG Alexa Fluor568 (secondary) antibody (1:200; Molecular Probes, Carlsbad, Calif.).

Immunohistochemistry of mouse tissues: Tissues were fixed in 10% formalin and processed for hematoxylin and eosin staining. All samples were examined by two experienced pathologists for typical inflammation signs in a blinded manner.

T-cell assays: MMC cells (Neu-positive) and splenocytes from syngeneic neu/CD46-transgenic mice (Neu-negative) were treated with mitomycin C at a final concentration of 50 μg/m for 20 min, and then washed extensively. Splenocytes from test animals (HDAd-αPDD-L1-γ₁ treated) and untreated control animals (naïve) were mixed 1:1 with mitomycin C treated cells and incubated for 1 day in the presence of 10 U/ml IL-2. Control splenocytes were also treated with PMA/ionomycin. IFNγ concentrations in the supernatant were measured by IFNγ ELISA (InVitrogen, cat #88-7214-22)

MicroRNA array analysis was performed by the UW Functional Genomics, Proteomics & Metabolomics Facility Core using Affymetrix miRNA 4.0 arrays

Real-time PCR: Total RNA was extracted from tumor infiltrating leukocytes, PBMCs, splenocytes and bone marrow cells using TRIzol™ per manufacturer's instructions (Invitrogen), then reverse transcribed to generate cDNA using QuantiTect Reverse Transcription Kit from Qiagen (cat #205311). The gDNA wipe-out reagent provided in the kit was used to eliminate potential genomic DNA contamination. Comparative real-time PCR was performed using Power SYBR Green PCR master mix (Applied Biosystems). The following primers were used: anti-mouse PDL1 forward (SEQ ID NO: 238), and reverse (SEQ ID NO: 239); mouse PPIA forward (SEQ ID NO: 240), and reverse (SEQ ID NO: 241); mouse RPL10 forward (SEQ ID NO: 189), and reverse (SEQ ID NO: 190).

Mouse PPIA was used as an internal control. A second internal control mouse RPL10 was also included and similar results were observed. Results were calculated according to 2^{(-ΔΔCt)} method and presented as percentage of relative expression, with setting the cDNA level of corresponding tumor samples as 100%.

Isolation of lineage-depleted (Lin⁻) bone marrow cells: For the depletion of lineage-committed cells, the mouse lineage cell depletion kit (Miltenyi Biotec, San Diego, Calif.) was used according to the manufacturer's instructions.

Colony forming unit assay. A total of 2500 Lin⁻ cells were plated in triplicates in ColonyGEL 1202 mouse complete medium (Reach Bio, Seattle Wash.) and incubated for 12 days at 37° C. in 5% CO₂ and maximum humidity. Colonies were enumerated using a Leica MS 5 dissection microscope (Leica Microsystems).

Cells: Mouse Mammary Carcinoma (MMC) cells were established from a spontaneous tumor in a neu/CD46-tg mouse. MMC cell authentication was performed by immunofluorescence using the Neu-specific monoclonal antibody 7.16.4 (Knutson et al., Cancer Res. 2004; 64: 1146-1151). TC-1 cells were from the American Type Culture Collection (ATCC, Manassas, Va.). TC-1 cells are immortalized murine epithelial cells that stably express HPV-16 E6 and E7 proteins. C57Bl/6-derived ovarian cancer ID8 p53^{-/-} brca2^{-/-} cells were described previously. Walton et al., Cancer Res. 2016; 76: 6118-6129. This cell line was generated by CRISPR/Cas9 knock-out of p53 and brca2 in 1D8 cells. MMC and TC-1 cells were maintained in RPMI-1640 supplemented with 10% fetal calf serum, 1 mmol/1 sodium pyruvate, 10 mmol/1 HEPES, 2 mmol/1 L-glutamine, 100 units/ml penicillin and 100 mg/ml streptomycin. 1D8 p53^{-/-} brca2^{-/-} cells were cultured in DMEM supplemented with 4% fetal calf serum, 100 μg/mL penicillin, 100 μg/mL streptomycin, and ITS (5 μg/mL insulin, 5 μg/mL transferrin, and 5 ng/mL sodium selenite). Absence of mycoplasma was confirmed using the PCR Mycoplasma Detection Kit from abm (Richmond, BC, Canada). For amplification cryopreserved cells were thawed and passaged four times.

Ovarian cancer biopsies were provided by the Pacific Ovarian Cancer Research Consortium (POCRC) Specimen Repository without any confidential information which would serve to identify a patient (Fred Hutchinson Cancer Research Center IRB protocol #6289). Tumor tissue from biopsies was dissected into 4 mm pieces and digested for 2 hours at 37° C. with collagenase/dispase (Roche) as described previously in Strauss et al. (PLoS One. 6: e16186, 2011). Leukocytes were isolated by magnetic activated cell sorting using human CD45 microbeads (Miltenyi Biotech, cat #130-045-801). Tumor-associated leukocytes from two high-grade serous ovarian cancer biopsies were pooled and RNA was analyzed by miRNA-Seq in comparison to matching PBMC RNA by LC Sciences, LLC (Houston, Tex.).

MicroRNA analyses: miRNA-Seq: Small RNA sequencing was performed as previously described (Valdmanis et al., Nat Med. 2016; 22: 557-562.). RNA was extracted using a miRNeasy mini kit (Qiagen Cat #1071023). 1 μg of RNA per sample was ligated to a 3′ Universal miRNA Cloning Linker (New England Biosciences cat #S1315) using T4 RNA Ligase 1 (New England Biosciences cat #M0204) in the absence of ATP. Ligated samples were run on a 15% urea-polyacrylamide gel. Fragments corresponding to small RNAs (17-28 nt) were cut from the gel and ligated to 5′ barcodes, again using T4 RNA ligase 1. Barcoded samples were then multiplexed and sequenced on an Illumina MiSeq machine obtaining 50 bp single-end reads, at the UW Center for Precision Medicine. The barcodes and adaptors were trimmed from the sequence and subsequently aligned to mouse microRNAs on miRBase using Bowtie version 0.12.7, allowing for 2 mismatches (Langmead et al., Genome Biol. 10: R25, 2009).

Northern blot for small RNA. This protocol is described in Valdmanis et al., Nat Med. 2016; 22: 557-562. The following ³²P-γ-ATP labeled probes were used: for miRNA 423-5p (SEQ ID NO: 235); for U6 snRNA (SEQ ID NO: 236). Radioactive RNA molecular weight markers were from Ambion.

Western blot: Tissue lysates were separated by SDS-PAGE and blots were incubated with chicken anti-HA-tag-HRP (Abcam, ab1190). Chemiluminescence detection on X-ray films was performed after treatment with Pierce™ ECL Plus Western Blotting Substrate (Thermo Fisher Scientific, cat #34029).

αPD-L1-γ₁ ELISA: Recombinant mouse PD-L1 protein (Sino Biological Inc, cat #50010-MO8H) at 2 μg/ml were used to coat ELISA plates. Serum from test animals was added at a 1:10 dilution and αPD-L1-γ₁ was measured using chicken anti-HA-tag-HRP (Abcam, ab1190).

Animals: All experiments involving animals were conducted were conducted with approval from the controlling Institutional Review Board and IACUC.

hCD46-transgenic mice: C57Bl/6 based transgenic containing the human CD46 genomic locus and expressing CD46 at a level and in a pattern similar to humans are described in Kemper et al. (Clin Exp Immunol. 124: 180-189, 2001). They were used in transplantation studies with C57Bl/6 derived TC-1 cells. Neu transgenic mice: Neu-tg mice (strain name: FVB/N-Tg(MMTVneu)202Mul) were obtained from the Jackson Laboratory (Bar Harbor, Me.). These mice harbor nonmutated, nonactivated rat neu under control of the mouse mammary tumor virus promoter (one transgene copy per genome). For in vivo transduction studies, CD46tg and neu-tg mice were crossed to obtain CD46^{+/+}/neu+ mice.

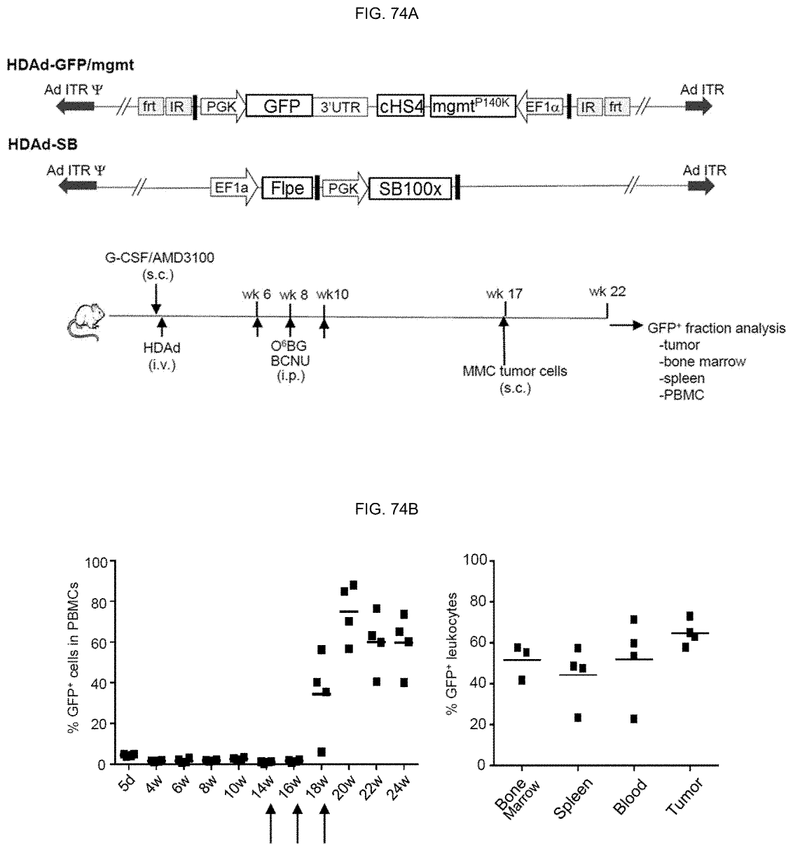

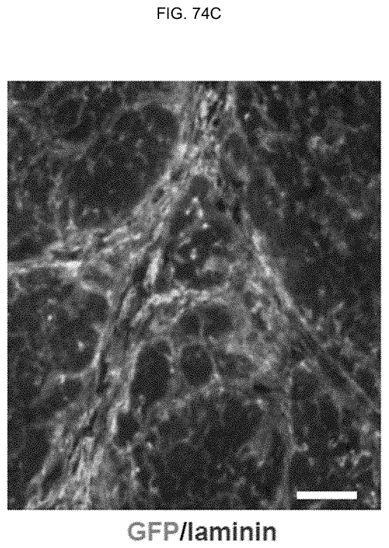

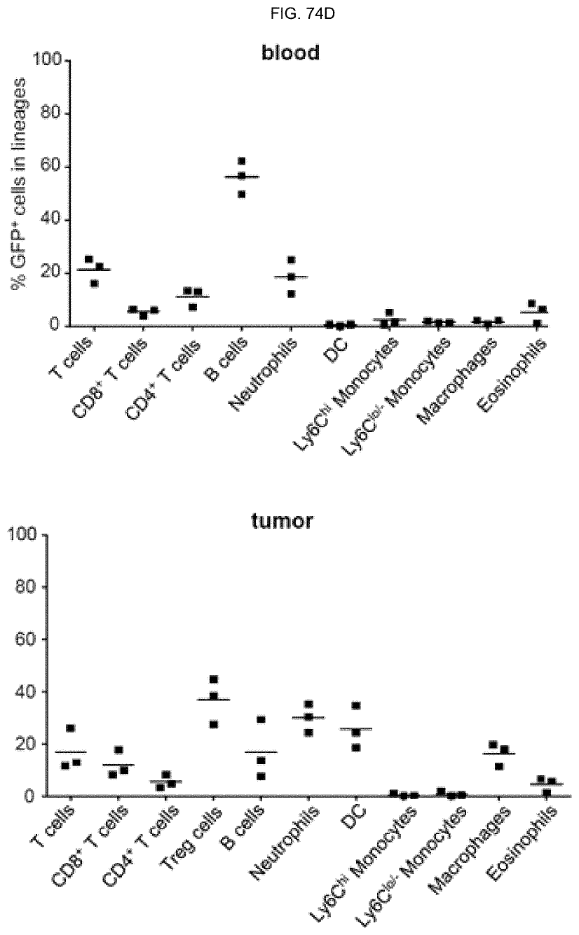

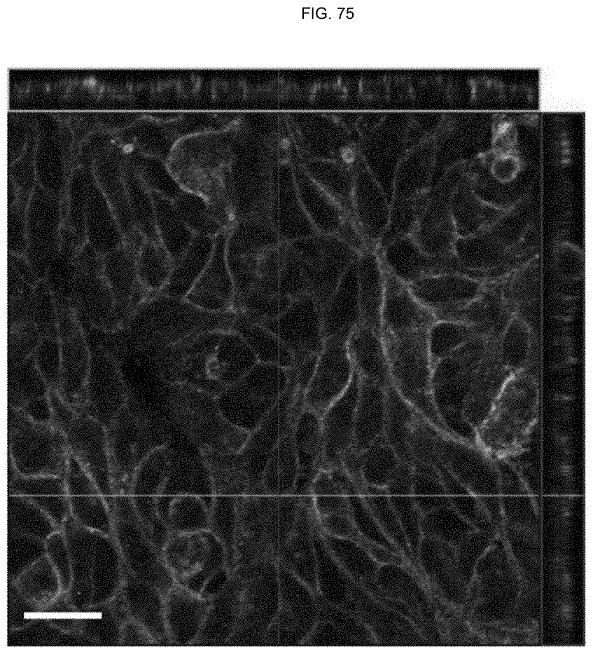

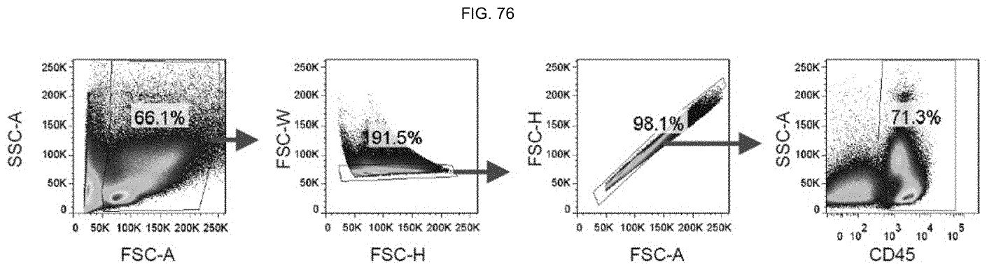

In Vivo HSPC Transduction/Selection: see FIG. 74A.

CD8 cell depletion: CDβ-T cells were depleted using intraperitoneal injection of 200 μg rat anti-mouse CD8 IgG (169.4; ATCC). Injection was repeated every 3 days to maintain the depletion.

Statistics: Statistical significance of in vivo data was analyzed by Kaplan-Meier survival curves and log-rank test (Graph Pad Prism Version 4). Statistical significance of in vitro data was calculated by two-sided Student's t-test (Microsoft Excel). P values >0.05 were considered not statistically significant (n.s.).

Results and Discussion.

Women who have at least one first-degree relative diagnosed with breast cancer before the age of 50 or with ovarian cancer at any age, are now referred to genetic testing. Using targeted capture and massively parallel genomic sequencing, a series of multi-gene tests have been established that detect germ-line mutations and predict the risk of cancer onset. Among these test platforms is BROCA (Walsh et al., Proc Natl Acad Sci USA. 108: 18032-18037, 2011, Shirts et al., Genet Med. 18: 974-981, 2016). Using BROCA, it has been established that more than one-fifth of ovarian and breast carcinomas are associated with inherited risk (Tung et al., Cancer. 121: 25-33, 2015). The problem is that the current options for prevention in high-risk carriers lag behind the constantly improving genetic diagnostics. Side effects of prophylactic salpingo-oophorectomy and mastectomy, including infertility, cardiovascular disease, osteoporosis, menopausal symptoms, and psychological effects, are expected throughout the woman's life. Use of serum markers such as CA125 and HE4 did not show significant reduction of ovarian cancer mortality (Jacobs et al., Lancet. 387: 945-95, 2016). Prophylactic vaccines against tumor-associated antigens like Her2/neu, HIF1α, or MUC1 rely on the presence of these antigens on all tumor cells, and are plagued by the development of antigen-loss mutants (Knutson et al., Cancer Res. 64:1146-1151, 2004).

The goal is to develop a long-lasting and technically simple approach that allows for the immuno-prophylaxis of cancer in patients with high-risk for tumor recurrence and, ultimately, in carriers of cancer-predisposing inherited mutations. During tumor progression, malignant cells secrete a number of specific chemokines that activate and mobilize HSPCs so that they enter the blood circulation and localize to the tumor where they are differentiated into tumor-supporting cells (Hanahan et al., Cell. 144: 646-674, 2011, Mantovani et al., Trends Immunol. 23: 549-555, 2002). HSPC-derived myeloid and lymphoid cells are present in early stages of cancer development (Okla et al., Front Immunol. 10: 691, 2019; Colvin, Front Oncol. 4: 137, 2014; Baert et al., Front Immunol. 10: 1273, 2019), for example in serous tubal intraepithelial carcinoma (STIC). Sarkar et al., Genes Dev. 31: 1109-1121, 2017. This approach is based on the genetic modification of hematopoietic stem cells. Because these cells are capable of self-renewal, a one-time intervention should have a life-long therapeutic effect. A minimally invasive and cost-efficient technology was developed that made in vivo gene delivery into HSPCs without leukapheresis, myeloablation and transplantation possible (Richter et al., Blood. 128: 2206-2217, 2016, Wang et al., J Clin Invest. 129: 598-615, 2019). The central idea of this approach is to mobilize HSPCs from the bone marrow using G-CSF/AMD3100, and while they circulate at high numbers in the periphery, transduce them with an intravenously injected HSPC-tropic helper-dependent adenovirus HDAd5/35++gene transfer vector system. These vectors use CD46, a receptor that is expressed on primitive hematopoietic stem cells. Transduced cells return to the bone marrow where they persist long-term. Novel features of the HDAd5/35++ vector system used in this study include: (i) CD46-affinity enhanced fibers that allow for efficient transduction of primitive HSPCs while avoiding infection of non-hematopoietic tissues after i.v. injection (including liver), (ii) a SB100X transposase-based integration system that functions independently of cellular factors and mediates random transgene integration without a preference for genes with one to two integrated vector copies per cell (FIG. 74A), and (iii) a MGMT^{P140K} expression cassette mediating selective survival and expansion of progeny cells without affecting the pool of transduced primitive HSPCs by short term treatment with low-dose O⁶BG/BCNU (Wang et al., Mol Ther Methods Clin Dev. 8: 52-64, 2018). The efficacy and safety of the in vivo HSPC gene therapy method in mouse models for hemoglobinopathies was recently demonstrated (Wang et al., J Clin Invest. 129: 598-615, 2019, Li et al., Blood. 131: 2915-2928, 2018). Here, this approach is used for prevention of cancer growth.

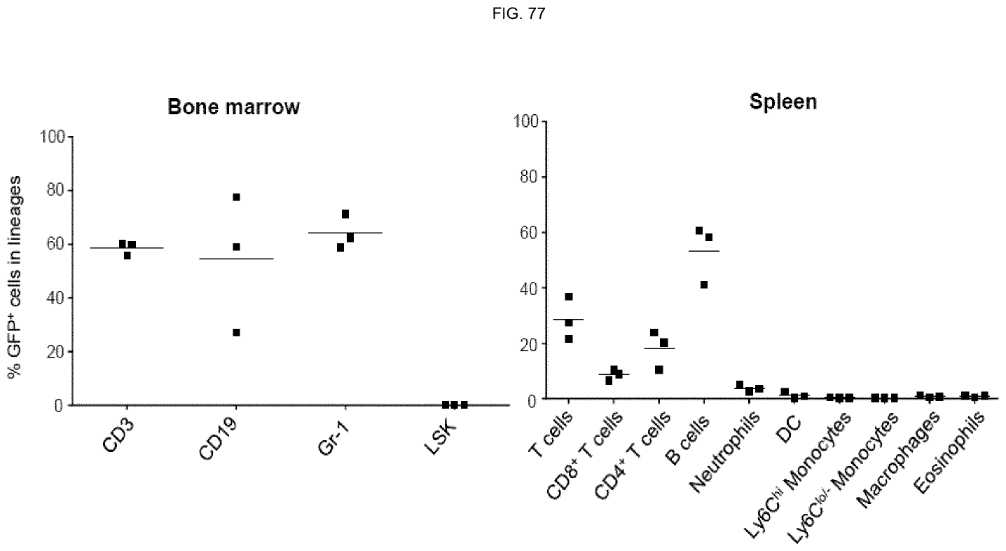

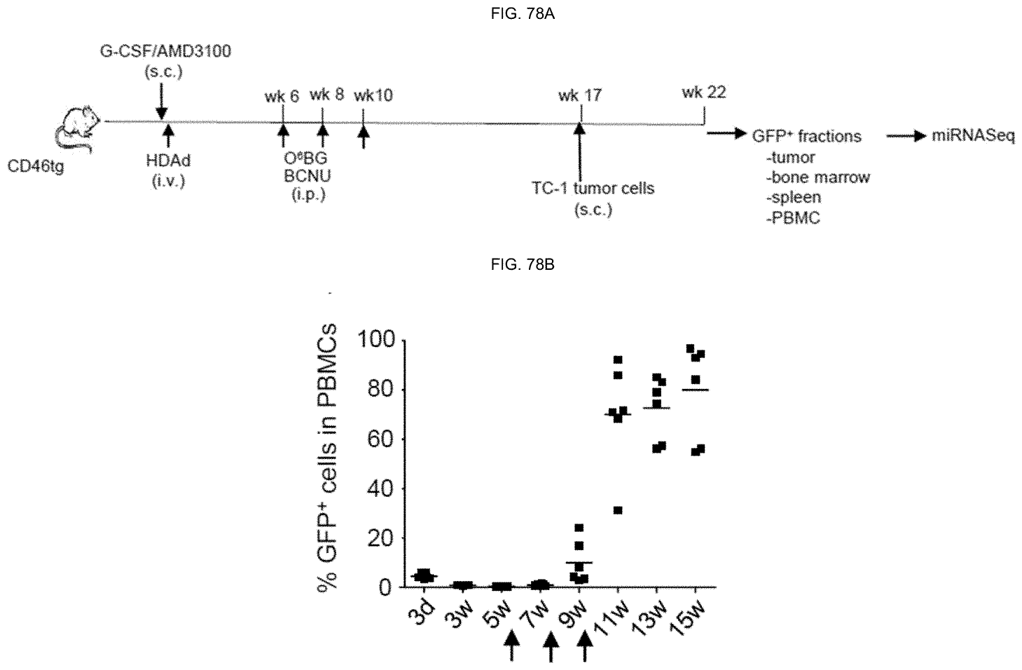

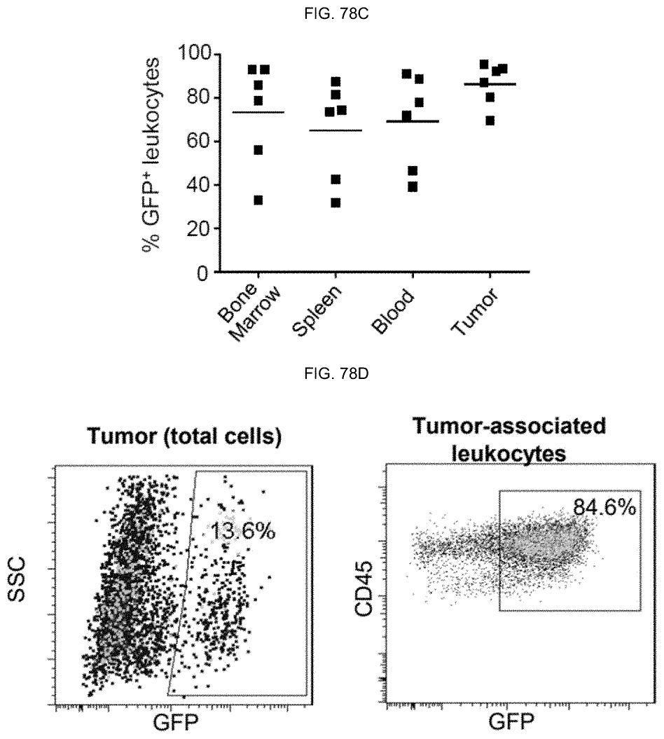

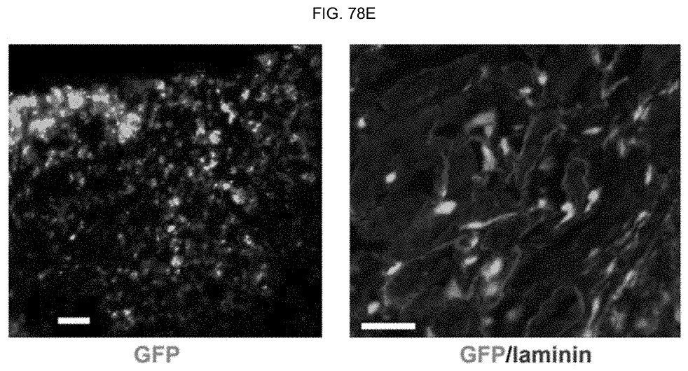

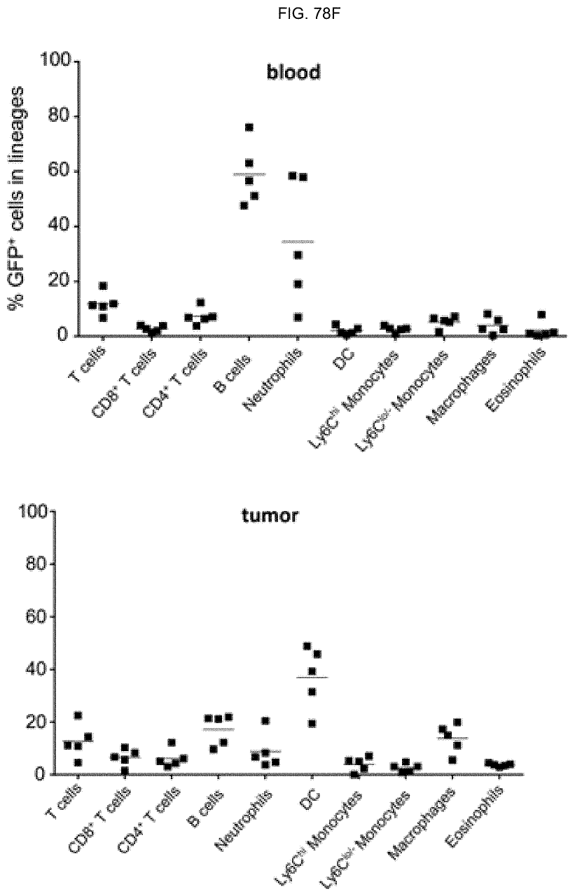

GFP expression in tumor-infiltrating leukocytes after in vivo HSPC transduction. Two human CD46 transgenic mouse models with syngeneic tumors were employed. (CD46 is required for HSPC transduction with HDAd5/35++ vectors). The first model included human CD46/rat neu-transgenic mice that overexpress rat neu in breast tissue from a mouse mammary tumor virus promoter. Neu-tg mice develop active immune tolerance towards Neu, which is dependent on Tregs and is similar to what is observed in breast cancer patients (Knuston et al., J Immunol. 177: 84-91, 2006). Mouse mammary carcinoma cells (MMC) are a Neu-positive breast cancer cell line derived from a spontaneous neu/CD46-transgenic mouse tumor (FIG. 75). HSPCs were mobilized in neu/CD46 tg mice and an integrating GFP-expressing HDAd5/35++ vector (FIG. 74A) was injected. Similar to previous studies (Wang et al., Mol Ther Methods Clin Dev. 8: 52-64, 2018), three rounds of low-dose treatment with O⁶BG/BCNU resulted in stable GFP expression in 80% of PBMCs (FIG. 74). At week 17 after in vivo HSPC transduction, syngeneic MMC cells were implanted into the mammary fat pad and tumor growth was monitored. When tumors reached a volume of 700 mm (Palmer et al., Methods in Molecular Biology, 2009:33-53), animals were sacrificed and GFP expression was analyzed. 80% of bone marrow cells, splenocytes, PBMCs, and tumor-infiltrating leukocytes expressed GFP (FIG. 74B). In the tumor, GFP+ cells were found predominantly in tumor stroma (FIG. 74C). Immunophenotyping showed that GFP+tumor-infiltrating cells were lymphocytes (predominantly Tregs), neutrophils, DCs/MDSCs, and macrophages (FIGS. 74D, 76). This pattern differed from that of GFP+ cells in peripheral blood (FIG. 74D), bone marrow and spleen (FIG. 77), indicating that tumors actively differentiate HSPCs into specialized pro-tumor cells. Efficient recruitment of in vivo transduced HSPCs to the tumor was further confirmed in a second model consisting of CD46tg mice and TC-1 cells, a HPV16 E6/E7-positive mouse lung cancer cell line (FIGS. 78A-78C).

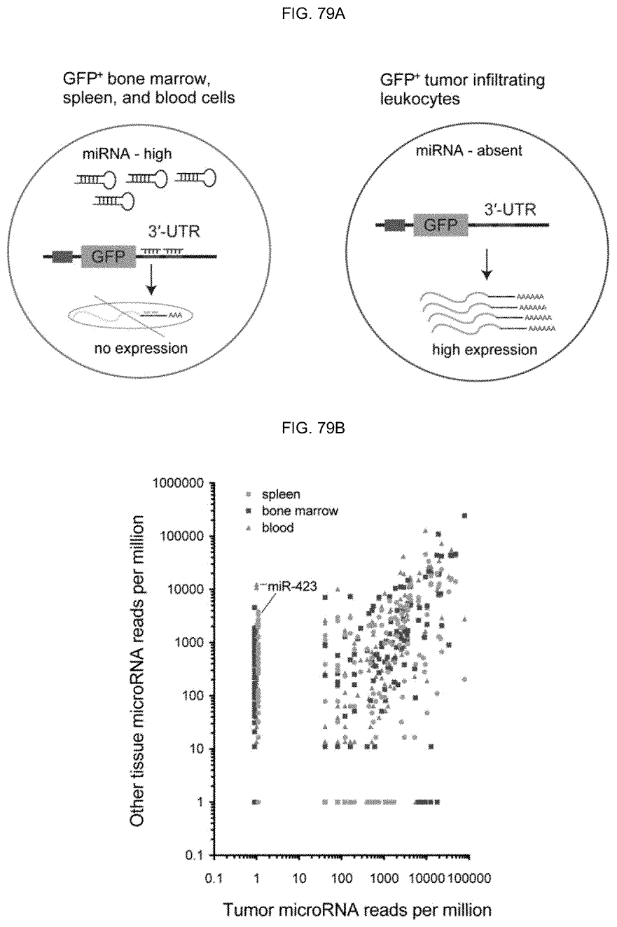

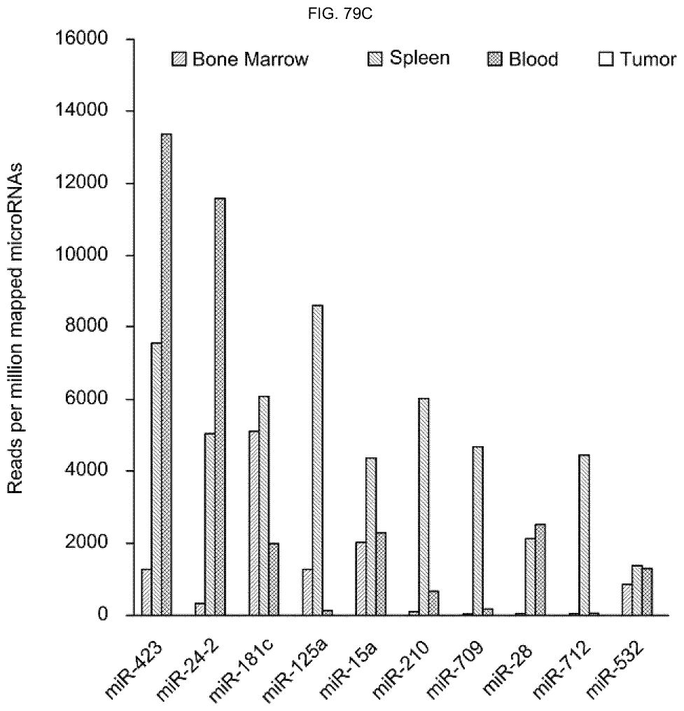

miRNA-regulated transgene expression in tumor-infiltrating leukocytes. FIG. 74B and FIG. 78C illustrate that GFP (under the control of the ubiquitously active EF1α promoter) is not only expressed in tumor-infiltrating leukocytes but also in other tissues including bone marrow, spleen, PBMC, and resident macrophages. To minimize auto-immune reactions, the therapy approach requires that the therapeutic transgene (i) be predominantly expressed in the tumor, (ii) automatically activate only when the tumor begins to develop, and (iii) cease when the tumor disappears. These requirements can be met through miRNA regulation. During hematopoiesis, the miRNA profile changes depending on the differentiation stage and cell lineage (Chen et al., Science. 2004; 303: 83-86). Tumor-associated myeloid cells have distinct mRNA and miRNA expression profile (Thorsson et al., Immunity. 48: 812-830 e814, 2018). Finally, there is a high degree of conservation of miRNAs in myeloid and lymphoid cells found in different tumor types in humans (Thorsson et al., Immunity. 48: 812-830 e814, 2018). The principle of miRNA regulation of transgene expression is shown in FIG. 79A. Using the in vivo HSPC-transduced mouse models, GFP+/CD45+ cells from bone marrow, spleen, PBMCs, and tumor were sorted (FIGS. 74B, 78C) and their miRNA expression profile was analyzed. The goal was to find miRNAs that were expressed at high levels in bone marrow, blood and spleen cells, but were absent in tumor-associated leukocytes. Total RNA (pooled from five mice) was subjected to next generation miRNA sequencing (FIGS. 79B, 79C). A series of miRNAs that fulfilled the above criteria were identified. miR423-5p, a miRNA that was on the top of the list, both in the neu/CD46tg-MMC (FIG. 79B) and in the CD46tg-TC-1 model (FIG. 79C) was focused on. miR-423-5p is conserved between humans and mice and could therefore be used in the further development of the approach towards the clinic. The expression profile of miRNA-423-5p in GFP+ fractions from in vivo transduced mice with MMC and TC-1 tumors was validated by microRNA array (not shown) and Northern Blot analysis (FIG. 81).

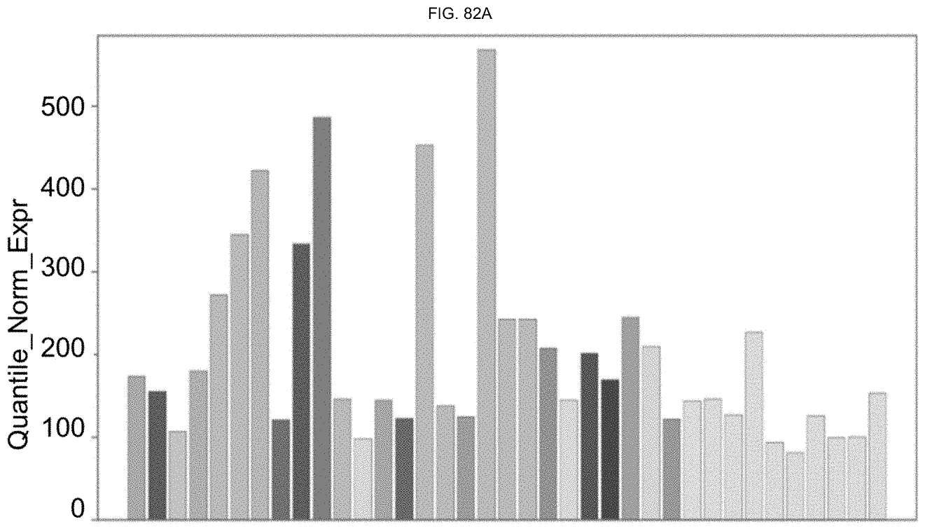

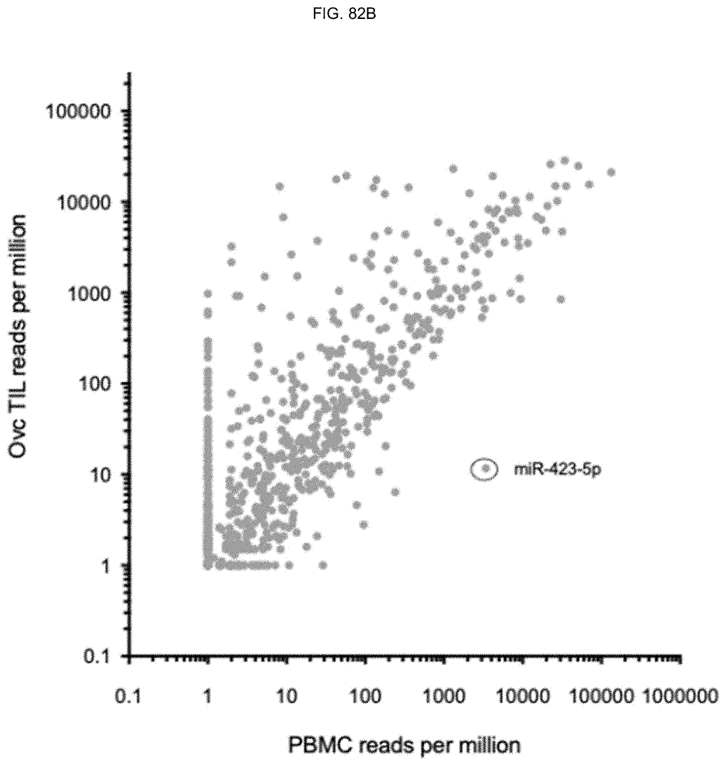

To assess whether miR-423-5p regulation could also be used in humans, levels of miR-423-5p in a published dataset that evaluated microRNAs across a series of human tissues were examined. Ludwig et al., Nucleic Acids Res. 2016; 44: 3865-3877. It was found that miR-423-5p is in the top 20% of expressed microRNAs and has even distribution across tissues, including in the bone marrow and spleen (FIG. 82A). Matching PBMCs and tumor biopsies were obtained from two patients with high-grade serous ovarian cancer. miRNA-Seq was performed on RNA from tumor-infiltrating (CD45+) leukocytes vs RNA from matching PBMCs (FIG. 82B). This analysis confirmed high-level expression of miR423-5p in PBMCs and low-level expression in tumor-infiltrating leukocytes. These data demonstrate that the results observed in mice have the strong potential to be translated to human studies.

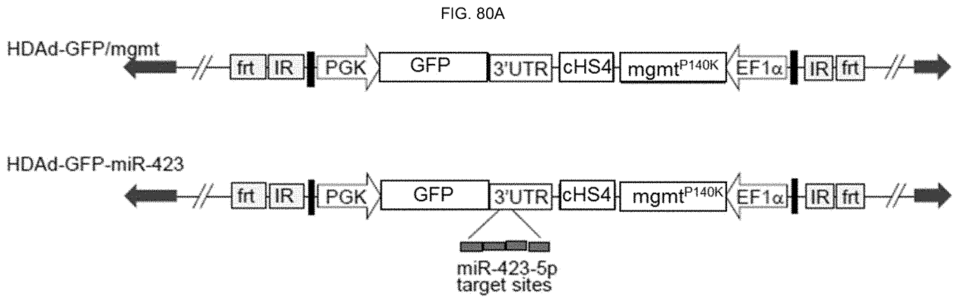

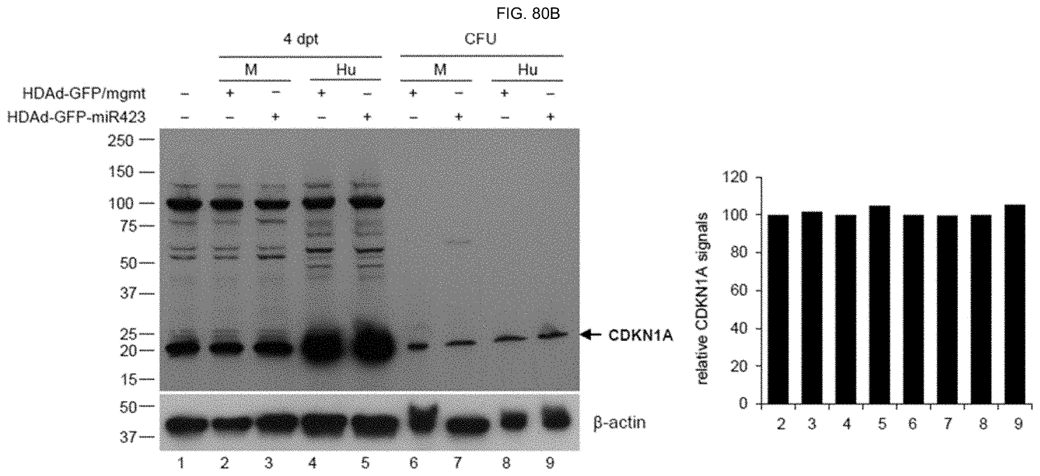

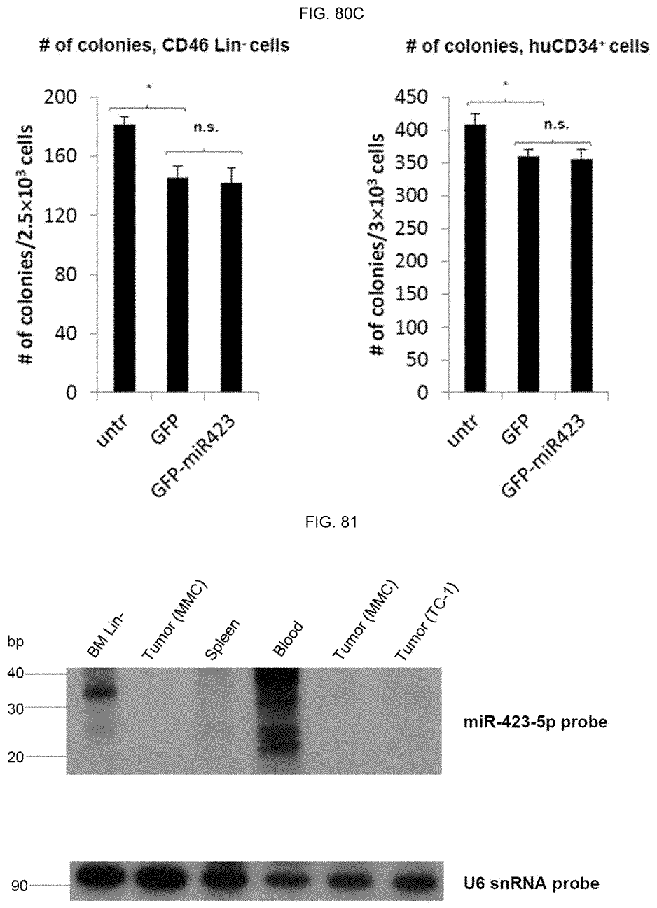

Effect of HDAd-mediated miR-423 target site expression on HSPCs. miRNA-423-5p is expressed in all normal tissues and therefore, most likely, involved in the regulation of gene expression. A search of target mRNAs for miR-423-5p in “mirtarbase” identified the cyclin-dependent kinase inhibitor 1A (CDKNIA) mRNA as the primary target (available online at mirtarbase.mbc.nctu.edu.tw/php/detail.php?mirtid=MIRT000589#target). Other target mRNAs include transcription elongation factor A like 1 (TCEAL1), bcl2 like 11 (bcl2L11), and proliferation-associated 2G4 (PA2G4). To assess whether added expression of miR-423-5p target sites from HDAd vectors influences the expression of CDKNIA, two HDAd-GFP vectors with and without the target sites linked to a GFP containing mRNA were constructed (FIG. 80A). Mouse and human HSPCs, i.e. cell types with high level miR-423-5p expression, were infected at MOIs that would result in the transduction of the vast majority of cells (Li et al., Mol Ther. 27(12):2195-2212, 2019) and analyzed CDKN1A protein levels three days later by Western blot (FIG. 80B). A significant difference between the two HDAd vectors in both cell types was not found. Furthermore, no detrimental effects of miR-423-5p target site overexpression were observed in progenitor colony assays (FIG. 80C). As outlined elsewhere herein, in vivo HSPC transduction with a therapy vector that contained the miR423-5p target sites did not cause abnormalities in hematopoiesis. Taken together, this suggests that the disclosed miR-423-5p-based regulation system is safe in HSPCs.

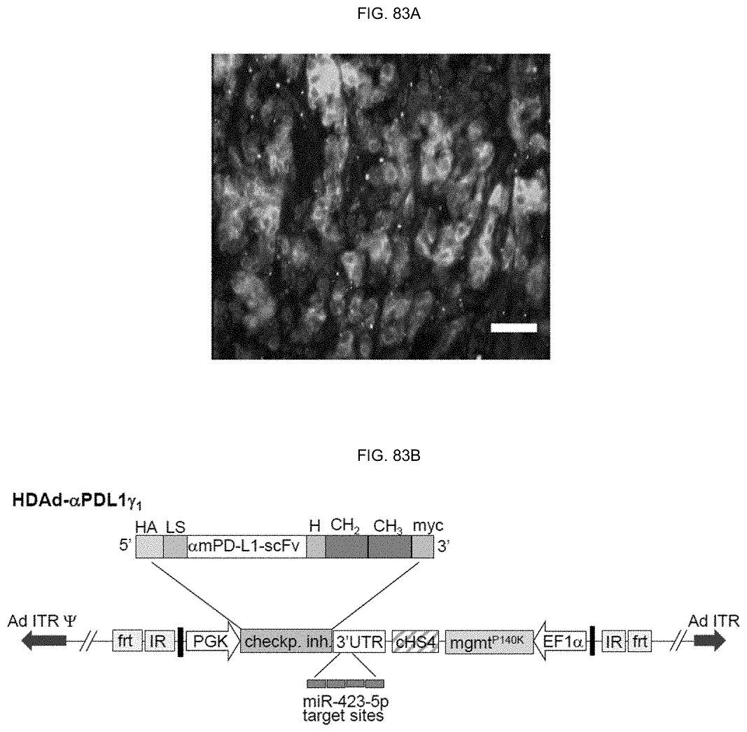

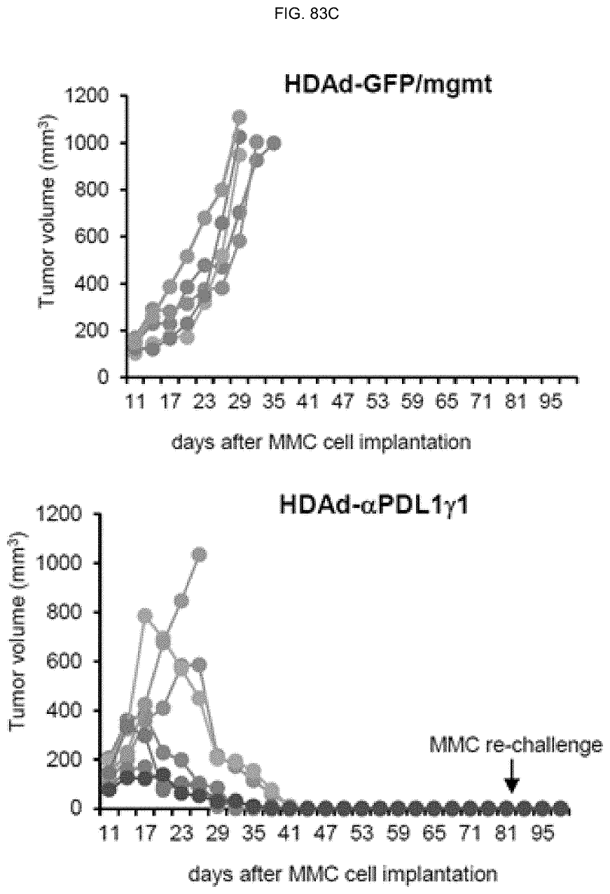

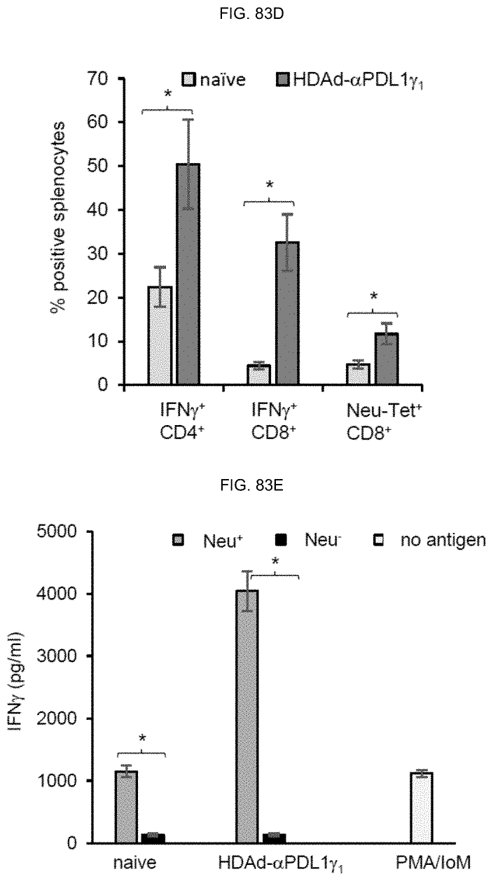

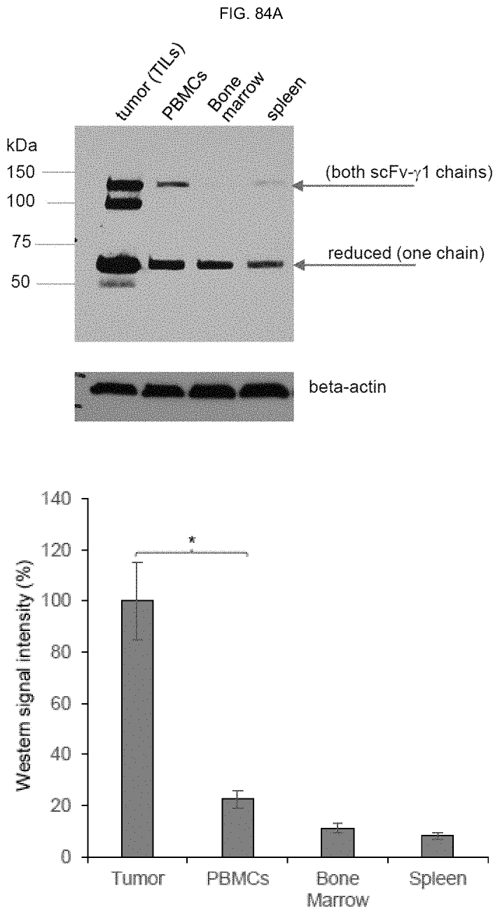

Immuno-prophylaxis study. In hereditary breast and ovarian cancer, genetic variants disrupt DNA repair mechanisms resulting in higher mutational burden and neoantigen presence. This makes the tumors more amenable to immunotherapies than non-heritable breast and ovarian cancers, which are often characterized by aberrant copy number and low immunogenicity (Thorsson et al., Immunity. 2018; 48: 812-830 e814). Here, the checkpoint inhibitor αPD-L1-γ1 was selected as the immunotherapeutic transgene. Previously, it was shown that intratumoral αPD-L1-γ1 expression after viral gene transfer resulted in tumor growth attenuation (Engeland et al., Mol Ther. 22: 1949-1959, 2014, Reul et al., Front Oncol. 9: 52, 2019). In MMC cell cultures, strong PD-L1 expression was observed (FIG. 83A), which should make MMC tumors susceptible to αPD-L1-γ1 therapy. Four copies of miR423-5p target sites were integrated into a globin 3′ UTR linked to the αPD-L1-γ1 gene (FIG. 83B). The experimental scheme was the same as shown in FIG. 74A. In mice that were in vivo transduced with the control HDAd-GFP/mgmt vector, implanted MMC tumors grew rapidly and reached the endpoint volume by day 35 after tumor cell transplantation (FIG. 83C, left panel). In the αPD-L1-γ1 model, after initial tumor growth, 6 out of 7 tumors regressed and did not recur within the observation period (100 days). Treated mice rejected another challenge of MMC cells given 11 weeks after the first injection. Depletion of CD8 cells by anti-CD8 mAb injections abolished the therapeutic effect. Anti-tumor T-cell responses were measured at the end of the observation period (day 100). Analysis of splenocytes by flow cytometry showed a significant higher percentage of interferon-γ (IFNγ)-producing CD4 and CD8 cells as well as a higher frequency of CD8 cells that stained positive with a Neu-tetramer (FIG. 83D). Splenocytes from HDAd-αPD-L1-γ1-treated animals exhibited a 30-fold greater IFNγ secretion upon stimulation with (Neu-positive) MMC cells, compared to Neu-negative cells (FIG. 83E). As expected, naïve CD46/neu-tg mice possessed Neu-specific T-cells, which however could not control tumor growth due to the presence of immunosuppressive T-cells in the tumor (Knutson et al., J Immunol. 2006; 177: 84-91).

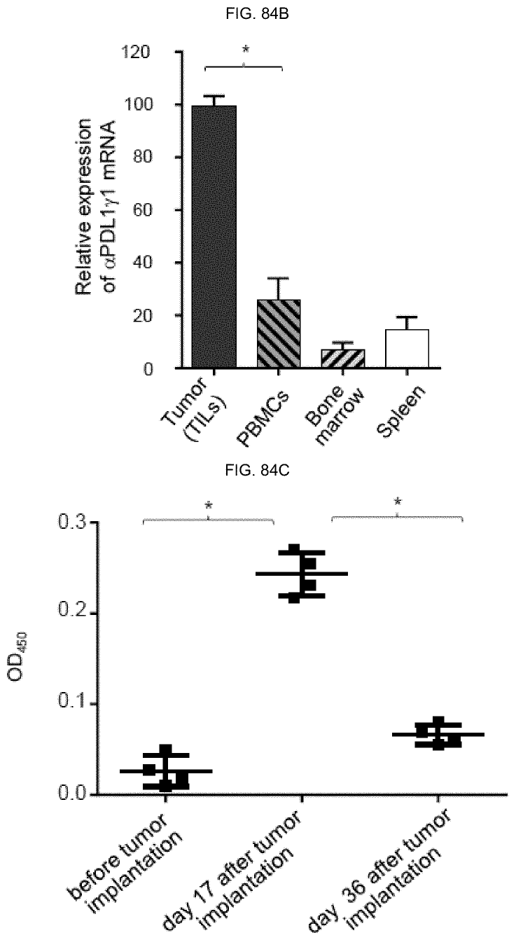

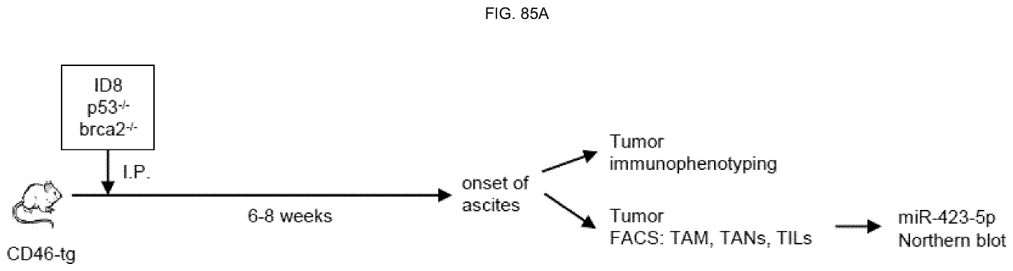

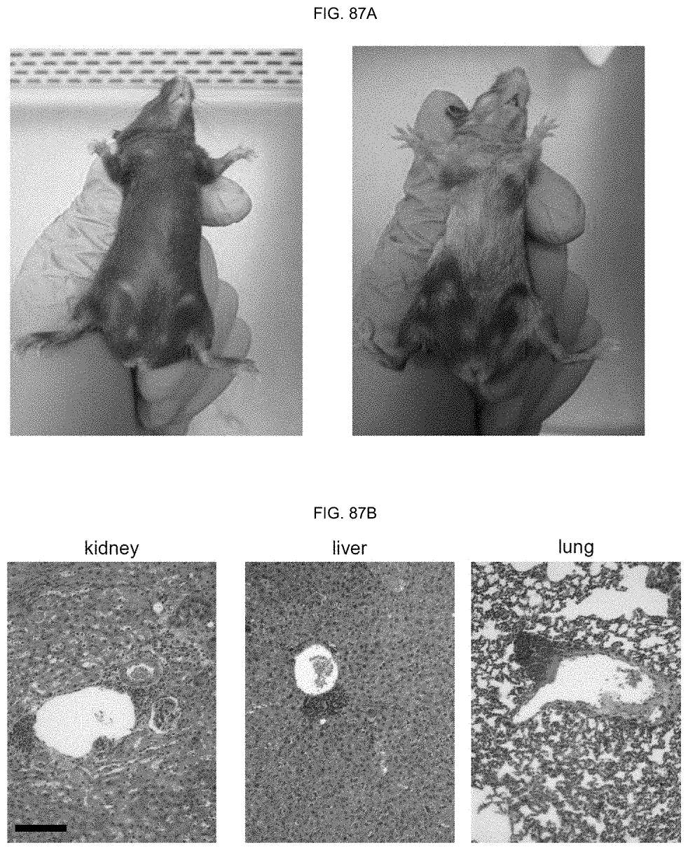

Kinetics and specificity of αPD-L1-yi expression in the MMC/neu-transgenic mouse model. In a separate group of HDAd-αPDLlyimiR423-treated animals, tumors were harvested at day 17 after implantation before they started to shrink. In these tumors (300-400 mm³), 10-fold higher levels of αPD-L1-γ₁ were observed in the tumor than in PBMCs, bone marrow, and spleen by Western blot analysis 8 (FIG. 84A). Preferential expression of αPD-L1-γ₁ mRNA in tumor-infiltrating leukocytes was confirmed by qRT-PCR (FIG. 84B). This expression pattern suggested that miR-423-regulation suppressed αPD-L1-γ₁ expression in HSPC progeny other than tumor-infiltrating myeloid and lymphoid cells. Serum αPD-L1-γ₁ became detectable after MMC cell injection and declined once tumors had disappeared, indicating a functional autoregulation of αPD-L1-γ₁ expression (FIG. 84B) i.e. the transgene expression started only once HSPGs differentiated into tumor-associated leukocytes. Starting from week 2 after MMC cell injection, auto-immune reactions were observed, reflected by fur discoloration and inflammatory infiltrates in tissues (FIG. 87, showing mice in FIG. 87A and samples of kidney, liver, and lung in FIG. 87B). Importantly, in animals sacrificed 4 weeks after tumor disappearance, the histology of all organs returned to normal. This observation indicates that as long as αPD-L1-γ₁ is expressed and released into the blood stream, transient auto-immune reactions (most likely against neu-expressing tissues/cell types) can occur. Notably, a study with a HDAd αPD-L1-γ₁ vector without miR-423-5p target sites had to be terminated because >20% weight loss occurring in treated animals two weeks after the last O⁶BG/BCNU treatment. This underscores the necessity for regulated αPD-L1-γ₁ expression. The observed auto-immune reactions could be minimized by physical tethering of αPD-L1-γ₁ to the tumor or by the use of intracellular immunomodulatory effectors (e.g. miRNAs that repolarize tumor-promoting leukocytes into tumor-killing cells). Furthermore, vectors could also contain a truncated EGFR receptor that allows for the destruction of all transduced cells by antibody (Erbitux)-dependent cytotoxicity (Wang et al., Blood. 2011; 118: 1255-1263).

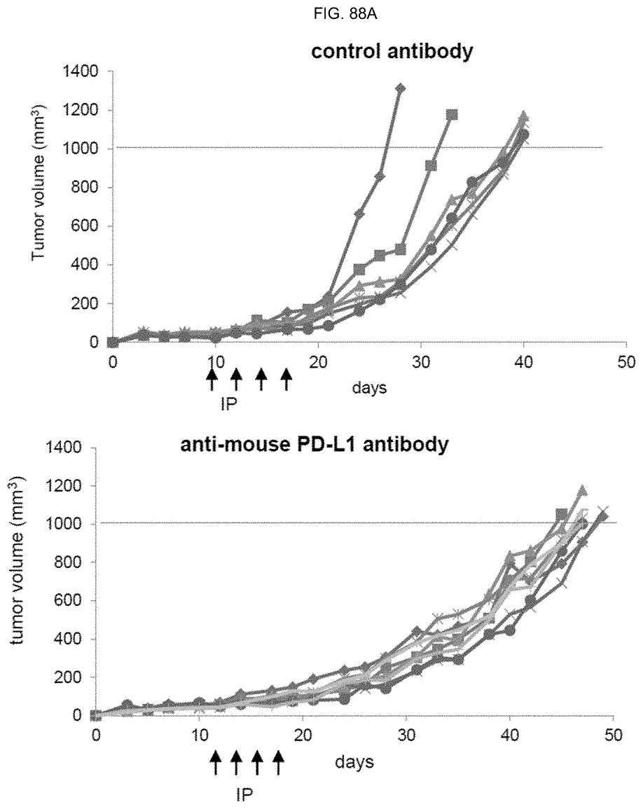

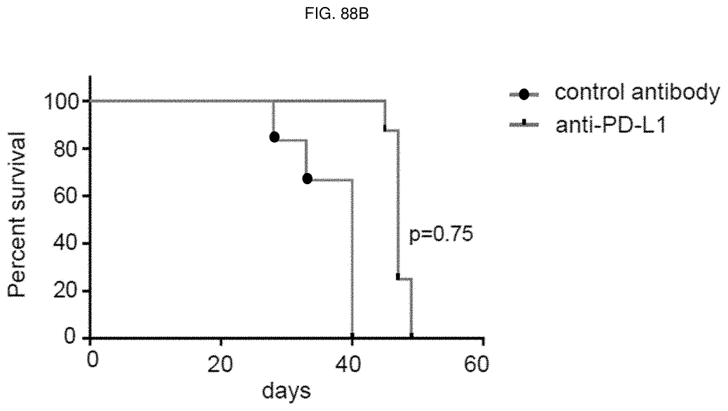

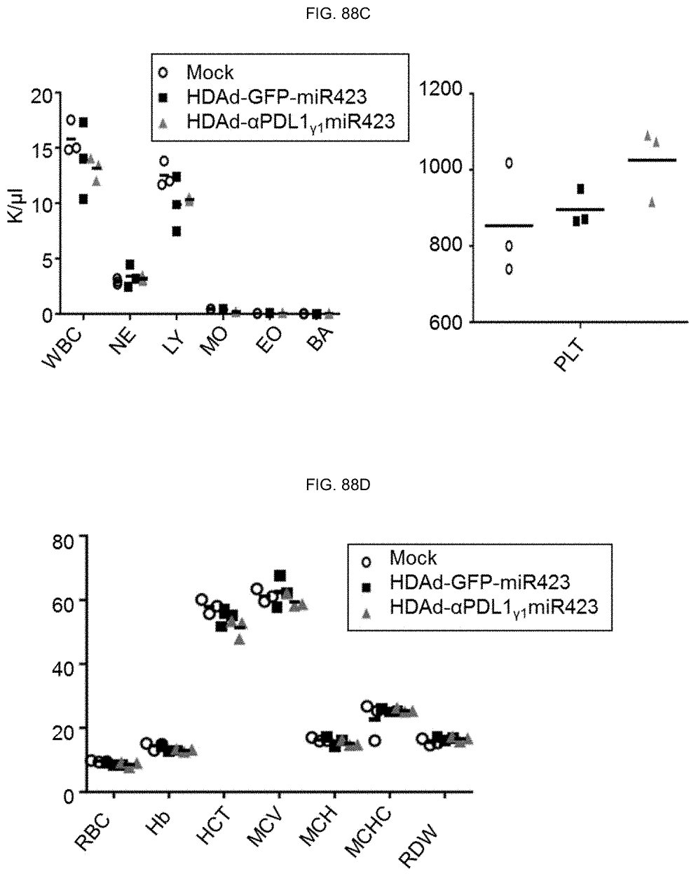

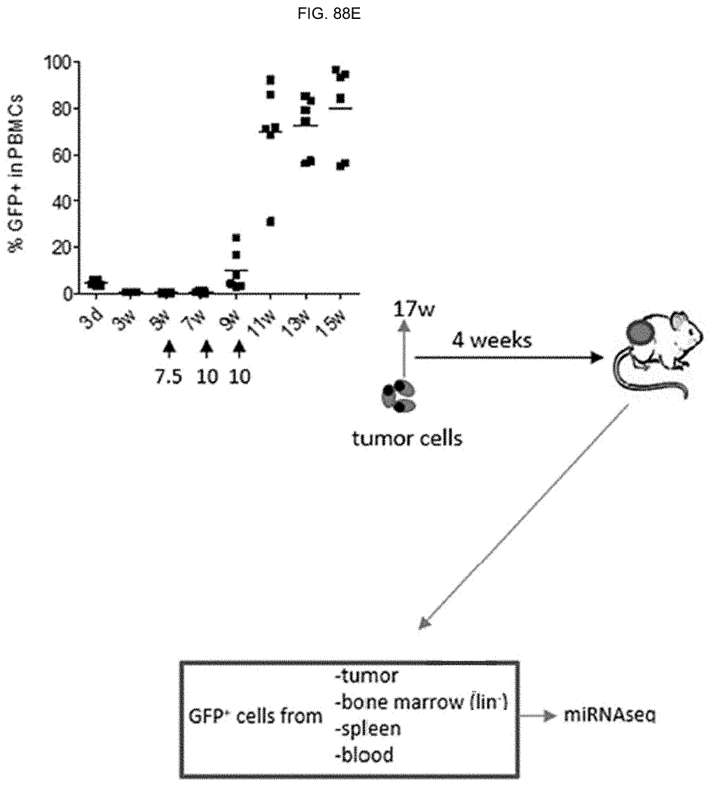

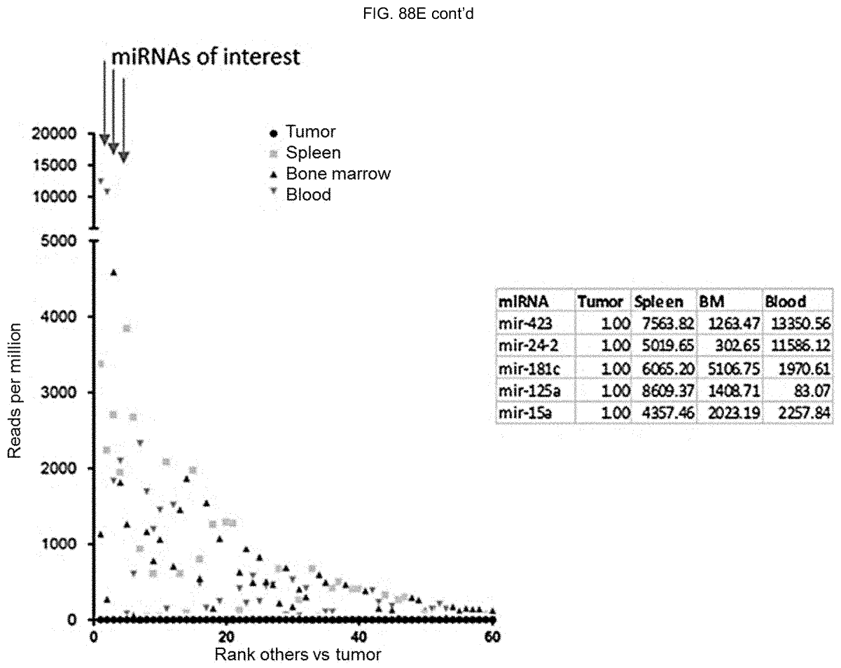

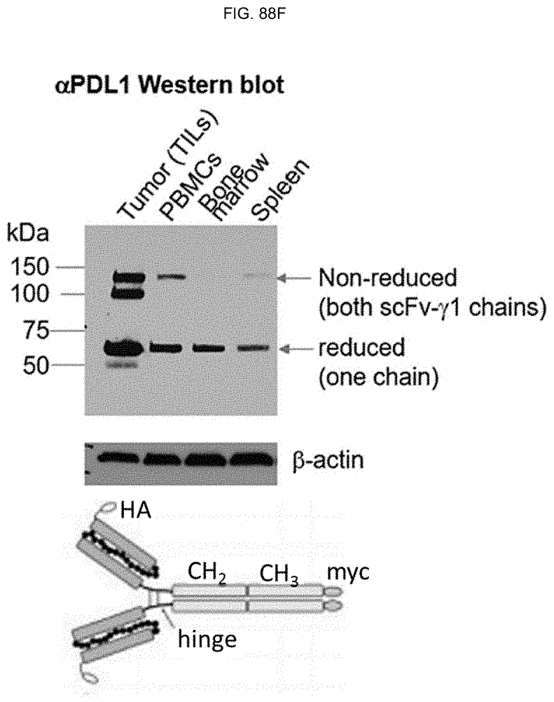

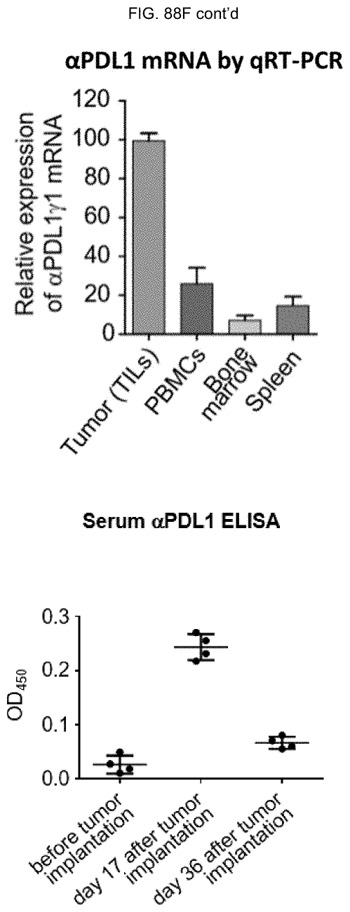

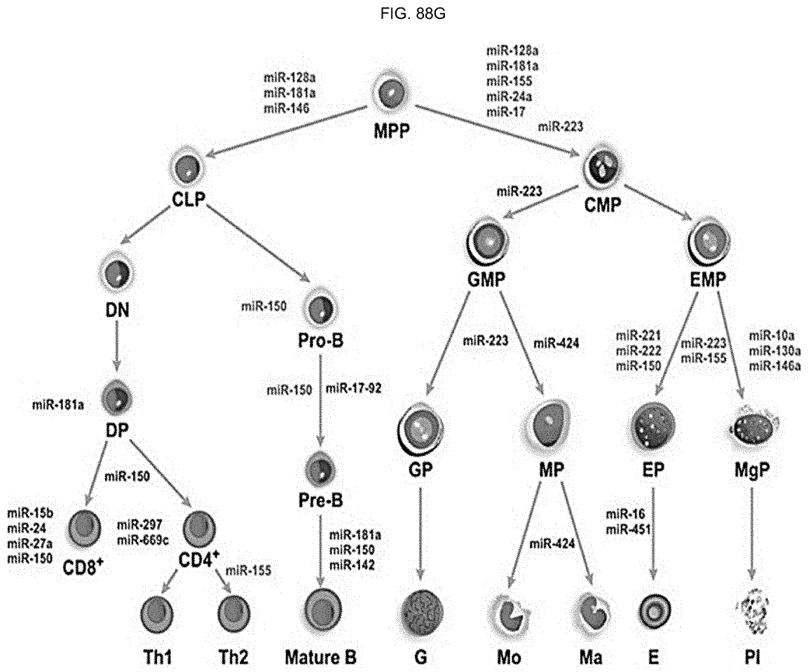

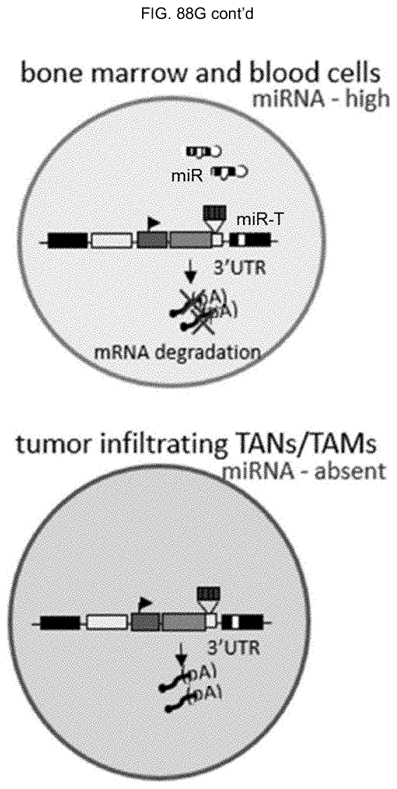

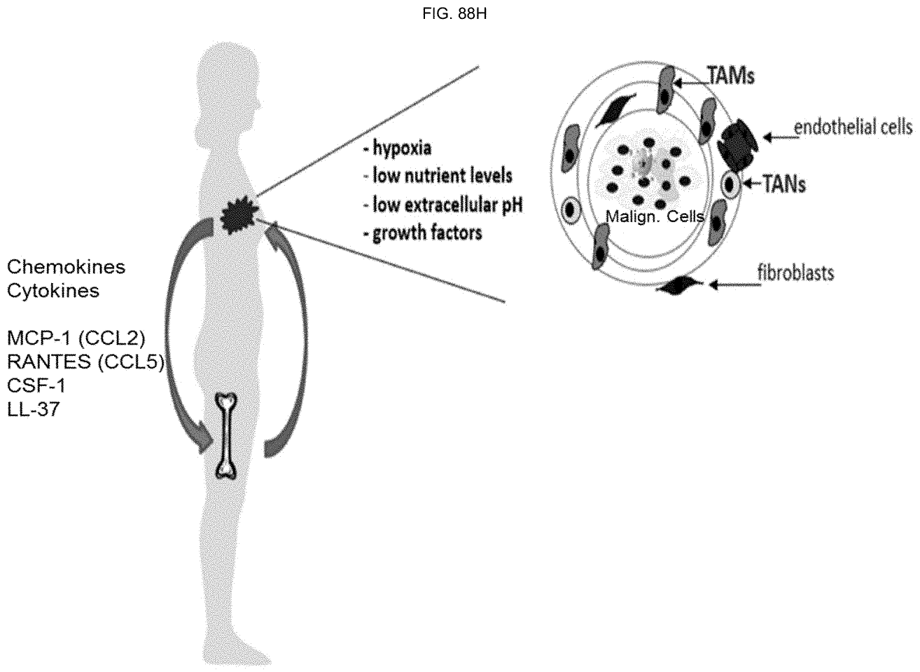

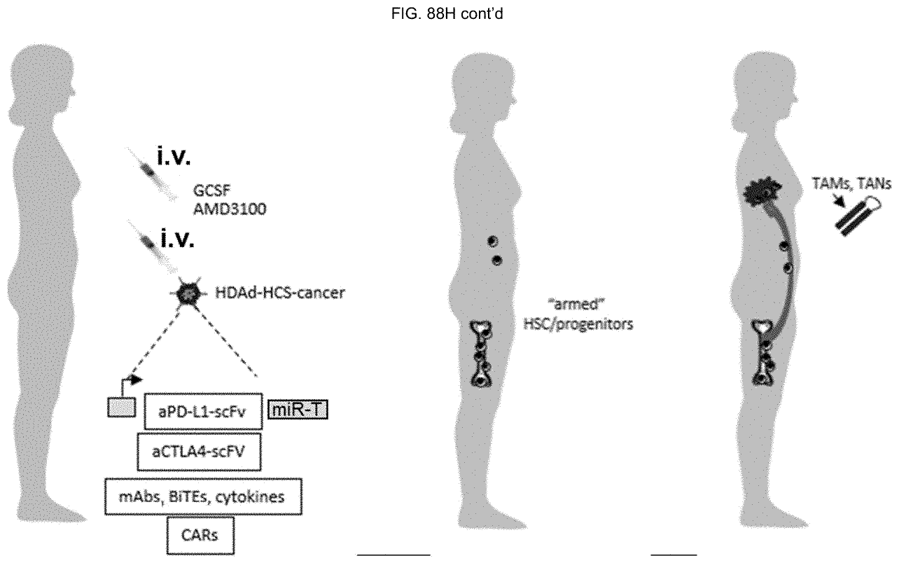

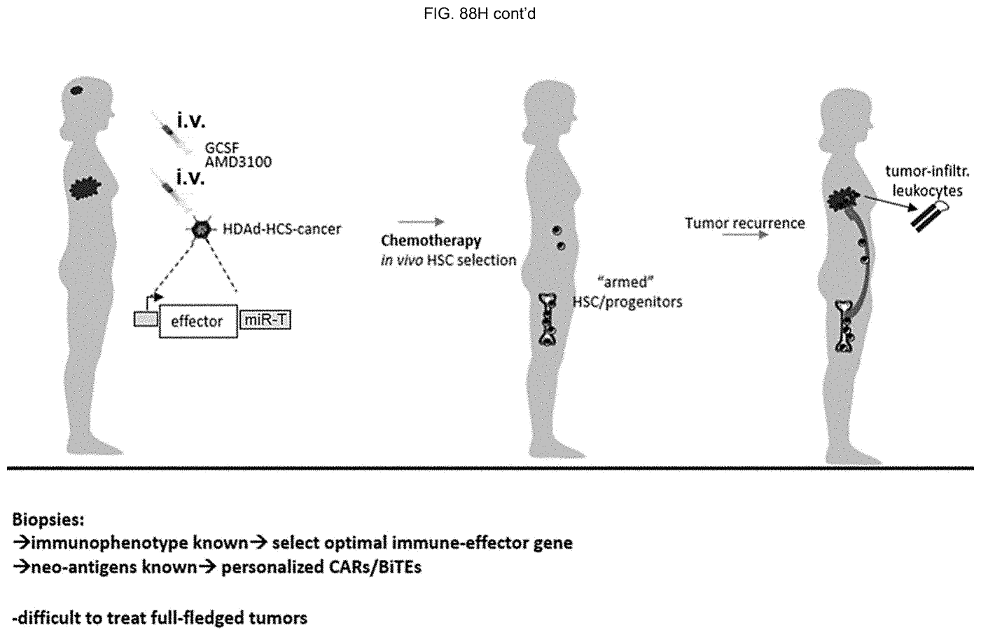

The efficacy of the in vivo HSPC αPD-L1-γ₁ gene therapy approach is remarkable considering that in the neu-tg/MMC model, other immunotherapy approaches did not prevent tumor recurrence (Knutson et al., Cancer Res. 64:1146-1151, 2004, Burgents et al., J Immunother. 33: 482-491, 2010). In this context, four rounds of intraperitoneal injection of an anti-mouse PD-L1 monoclonal antibody had no significant effect on tumor growth (FIGS. 88A, 88B). These data indicate that intratumoral expression of αPD-L1-γ₁ 1 early during tumor development (as soon as HSPC progeny cells infiltrate the tumor) can tip the balance between suppressor and effector immune cells towards tumor elimination.

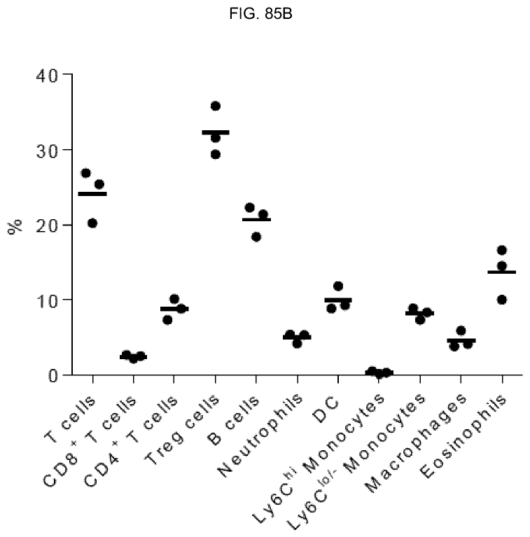

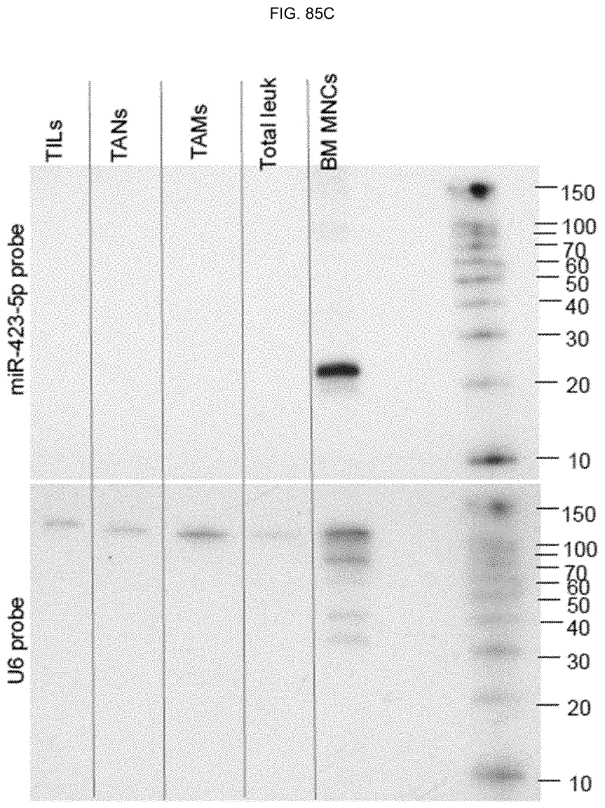

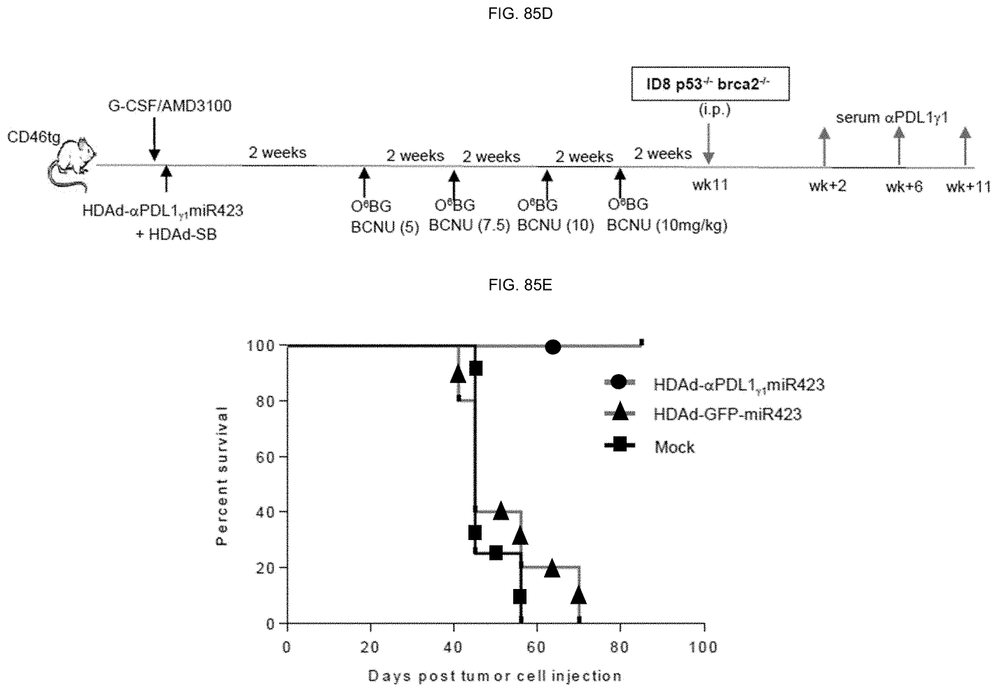

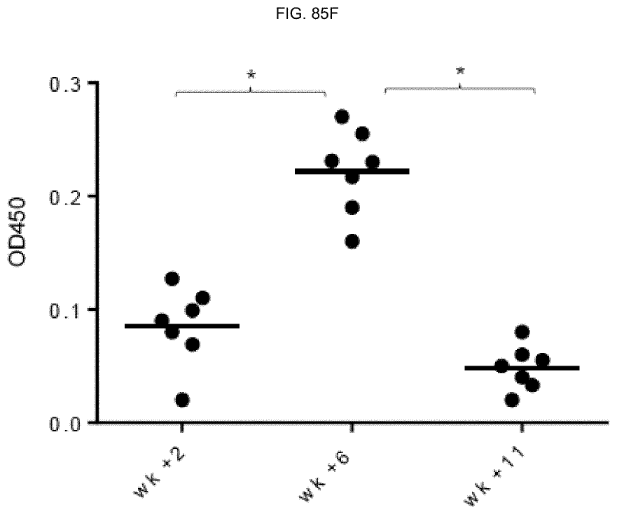

Immunoprophylaxis and therapy studies in an ovarian cancer model with p53 and brca2 mutations. C57Bl/6 derived murine ovarian cancer ID8 cells do not contain typical cancer-associated germ-line mutations (brca1, brca2, p53, Nf1, Rb1, Pten . . . ) and poorly form tumors after intraperitoneal injection. Walton et al., Cancer Res. 76: 6118-6129, 2016. Newer improved ID8-derived models, created by CRISPR/Cas9 knockout of tumor-suppressor genes, address these deficiencies. Walton et al., Cancer Res. 2016; 76: 6118-6129; Walton et al., Sci Rep. 2017; 7: 16827. Among these models are ID8-p53^{-/-}-brca2^{-/-} cells. Intraperitoneal injection of 2×10⁶ ID8-p53^{-/-}-brca2^{-/-} cells into CD46-transgenic mice resulted in tumor growth and onset of ascites (or death) within 6-8 weeks (FIGS. 84C and 85A). Intraperitoneal tumors were widespread along the mesenterium with invasion of other organs (spleen, liver, lymph nodes). Immunophenotyping of tumor-infiltrating leukocytes in intraperitoneal ID8-p53^{-/-}-brca2^{-/-} tumors showed the pronounced presence of Tregs as well as immunosuppressive DCs/MDSCs as well as TAMs (FIG. 85B). Tumor infiltrating T-cells (TILs), macrophages (TAMs), and neutrophils (TANs) were isolated from peritoneal ID8 p53^{-/-} brca2^{-/-} tumors and miRNA-423-5p levels were analyzed by Northern blot. As observed in the MMC and TC-1 models, miR-423-5p was expressed in bone marrow mononuclear cells but not detectable in tumor-infiltrating leukocytes including TILs, TANs, and TAMs) indicating that all three cell types had been specifically reprogrammed by the tumor (FIG. 85C).

First, the ID8-Trp53^{-/-}-brca2^{-/-} model was used in a prophylactic setting (FIG. 85D). After HSPC in vivo transduction/selection with HDAd-αPDL1γ₁miR423+HDAd-SB or HAd-GFP-miR423+HDAd-SB (control), ID8-p53^{-/-} brca2^{-/-} cells were injected intraperitoneally and serum αPDL1γ1 levels and onset of morbidity and ascites were monitored. While all control mice reached the endpoint by day 70 after in vivo transduction, 100% of HDAd-αPDL1γ₁miR423+HDAd-SB treated animals were alive at the end of the monitoring period (11 weeks after tumor cell inoculation) (FIG. 85E). Elevated serum αPDL1γ1 levels around week 6 (post cell injection) suggest that tumors had grown and activated serum αPDL1γ1 expression (FIG. 85F). By week 11, serum αPDL1γ1 returned to background levels indicating that tumors had been cleared. In this study, signs of auto-immune reactions (e.g. fur discoloration) were not observed, most likely due to the absence of antigens shared between the tumor and normal tissues (e.g. Neu). In the context of assessing the safety of the described approach, it was also shown that in vivo HSPC transduction with HDAd-αPDL1γ₁miR423 did not cause abnormalities in hematopoiesis (FIGS. 88C, 88D). In mice implanted with syngeneic tumor cells, percentage of GFP positive cells in PBMCs was measured at indicated time points, and GFP positive cells were harvested for miRNAseq (FIG. 88E). Results identified miRNAs with expression patterns of interest (FIG. 58E). Western blot for PDL1 in tumor (TILs), PBMCs, bone marrow, and spleen are shown and quantified as expression relative to mRNA in FIG. 88F. Serum αPDLA ELISA OD45o before tumor implantation and at indicated time points after implantation are shown are also shown in FIG. 88F. Schematic representations are shown in FIGS. 88G and 88H.

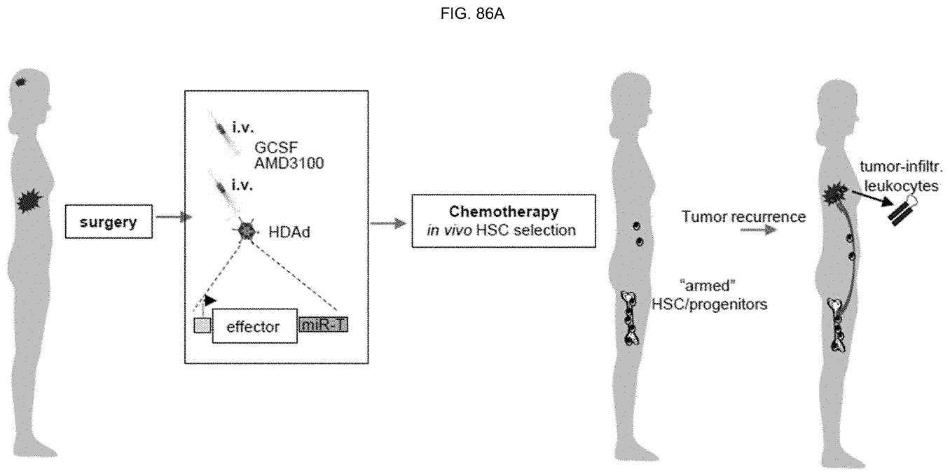

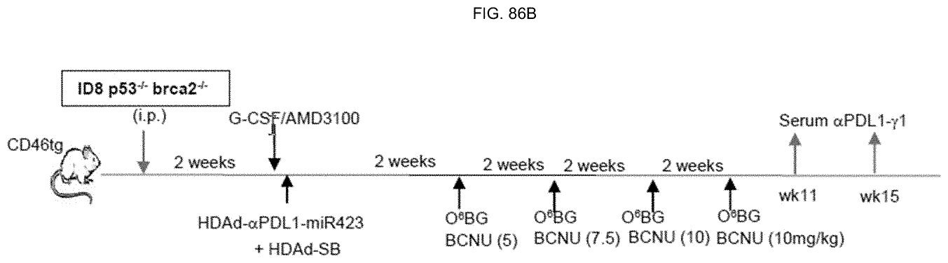

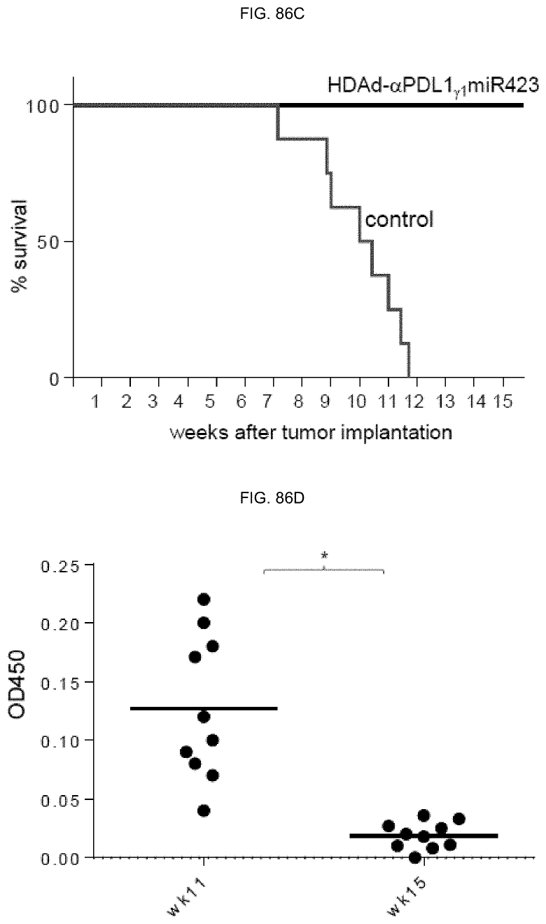

While a prophylaxis approach has the advantage of commencing automatically at a very early stage of tumor-development, its immediate application in healthy women carrying high-risk mutations will likely face regulatory hurdles in clinical translation. A more realistic goal, therefore, is to use this approach to prevent cancer recurrence after first-line therapy. In this case, in vivo HSPC selection can be directly embedded into the chemotherapy treatment of patients. FIG. 86A shows how in clinical setting, in vivo HSC transduction will start after surgical tumor debulking, or, if surgery is not an option, together with chemotherapy. O⁶BG/BCNU in vivo selection can be combined with chemotherapy. As a result of in vivo HSPC transduction/selection, armed HSPCs will lay dormant until cancer recurs which will trigger HSPC differentiation and activation of effector gene expression. This setting also has the advantage that tumor-specific neo-antigens and the immuno-phenotype of the tumor will be known from the analysis of surgical biopsies, which would allow for selecting the adequate immunotherapy effector genes. On the other hand, preventing the recurrence of cancer with “fully fledged” cancer hallmarks (Hanahan et al., Cell. 2011; 144: 646-674) is more challenging than targeting a tumor at early stages of development.

To simulate such a “therapeutic” setting, CD46-transgenic mice were injected first with ID8-Trp53^{-/-}-brca2^{-/-} cells followed by in vivo HSPC transduction/selection two weeks later (FIG. 86B). While all mice in the control setting (HDAd-GFP-miR423+HDAd-SB transduced HSPCs) reached the end point by week 12 after tumor cell injection, all mice treated with the αPDL1-γ₁ expressing vector were healthy at week 15 (FIG. 86C). As in the prophylaxis study, elevated serum αPDL1-γ₁ levels at week 11 suggest that tumors initially grew but disappeared once the self-regulated αPDL1-γ₁ mechanism was activated (FIG. 86D). These data indicate that the described approach could prevent cancer recurrence after surgery/first-line chemotherapy.

An mRNA profiling/Northern blot analyses for tumor-infiltrating leukocytes present in TC-1 (mouse lung-cancer) tumors (FIGS. 78A-81), MMC (mouse breast cancer) tumors (FIGS. 79A-79C and 81), and ID8-p53^{-/-}/brca2^{-/-} (mouse ovarian cancer tumors) (FIG. 85C) was performed. It was found in all three tumor types that miR423-5p is undetectable, but present at high-levels in normal hematopoietic compartments. Together with the data from human ovarian cancer biopsies (FIGS. 82A, 82B), this indicates that the miR423-5p-based system can be broadly used for different tumor types across species for the regulation of effector gene expression.

Considering the limited prophylactic options that are currently offered to women with germ-line mutations associated with high-risk of cancer onset, and the increasing numbers of these carriers due to population-wide screening, this in vivo HSPC gene therapy approach is a promising strategy that addresses a major medical problem.

### Example 7. In Vivo HSC Gene Therapy Using Erythroid Cells as a Factory for High-Level Production of a Secreted Therapeutic Protein

This example shows expression of a non-erythroid protein in erythroid cells and storage of that expressed protein in mature red blood cells after in vivo HSC transduction/selection. This system can be used to provide life-long therapeutic correction after a single intravenous intervention. At least some of the information contained in this example was published in Wang et al. (Blood Adv 3(19): 2883-2894, 2019; e-pub Oct. 4, 2019).

2.4 million new erythrocytes are produced per second in human adults, Nearly a quarter of the cells in the human body are red blood cells (Pierige et al., Adv Drug Deliv Rev. 60(2):286-295, 2008). In the process of erythropoiesis HSCs differentiate through common myeloid progenitors and pre-erythroblasts to orthochromatic erythroblasts (based on Wright's stain). At this stage, the nucleus is expelled, and the cells exit the bone marrow into the circulation as reticulocytes. 0:5% to 2.5% of circulating red blood cells in adults (1×10⁵/μl) and 2% to 6% in infants are reticulocytes. Reticulocytes are still capable of producing hemoglobin from mRNA. After one to two days, these ultimately lose all organelles and become mature red blood cells, which are not capable of protein biosynthesis anymore. Differentiation from committed erythroid progenitors to erythrocytes takes 7 days. Erythrocytes have a lifespan of 120 days. Old and dying erythrocytes are removed by the phagocytic system of the spleen.

Once HSCs have differentiated into committed erythroid cells, enormous amounts of α and β globin chains are produced and then later stored in erythrocytes as tetrameric hemoglobin. A healthy individual has 12 to 20 grams of hemoglobin per 100 ml of blood and 95% of the erythrocyte weight is hemoglobin (270×10⁶ Hb molecules per cell). The basis for this efficient biosynthesis is strong erythroid specific locus control regions (LCRs) that allow for high-level transcription and stable mRNA that is efficiently translated.

The tremendous speed and efficacy of erythropoiesis and the powerful machinery for hemoglobin production was used to produce non-erythroid secreted proteins from erythrocyte precursor cells (encompassing the differentiation stages from proerythroblasts to reticulocytes). Transgenes were under the control of a mini-β-globin LCR and contained 5′UTR regions of the β-globin gene for mRNA stabilization, To allow for long-term life-long production of therapeutic proteins, the gene transfer vectors targeted primitive HSCs. The in vivo HSC transduction approach involves G-CSF/AMD3100-triggered mobilization of HSCs from the bone marrow into the peripheral blood stream and the intravenous injection of an integrating, helper-dependent adenovirus vector system. Transgene integration is achieved (in a random pattern) using a hyperactive Sleeping Beauty transposase (SB100x), however, in particular embodiments, could be achieved through homology directed repair.

As a proof or principle that erythroid cells can be used for high-level production of therapeutic proteins that are secreted into the blood circulation, the focus herein was on a bioengineered form of coagulation factor VIII. The outcome of the study is relevant for hemophilia A treatment. Recently, clinical advancements have been made using recombinant adeno-associated virus (rAAV)-based gene therapy for liver-directed factor IX gene transfer for hemophilia B (High et al., Methods Mol Biol. 2011; 807:429-457). Preclinical studies also demonstrated the feasibility of treating hemophilia A with FVIII expressing rAAV vectors in animal models (Brown et al., Mol Ther Methods Clin Dev. 1:14036, 2014, Callan et al., PLoS One. 11(3):e0151800, 2016, Greig et al., Hum Gene Ther. 28(5):392-402, 2017). However, the widespread application of liver-directed rAAV hemophilia A gene therapy could face several obstacles: (i) the mostly episomal nature of rAAV genomes in hepatocytes and their loss due to cell division, specifically in children. (ii) the high cost of rAAV vector production, (iii) the limited packaging capacity of rAAV which cannot accommodate large transcriptionally regulatory elements often required to prevent gene silencing or genotoxicity (Grieger et al., J Virol. 79(15):9933-9944, 2005, Chandler et al., J Clin Invest. 125(2):870-880, 2015), and (iv) the increased risk of tumorigenicity due to potential rAAV integration near proto-oncogenes (Russell et al., Nat Genet. 2015; 47(10):1187-1193), specifically in patients with underlying liver disease, such as viral hepatitis, or in children with actively dividing hepatocytes, which represent a large fraction of hemophilia patients (Nault et al., Mol Cell Oncol. 3(2):e1095271, 2016, Nault et al., Nat Genet. 47(10):1187-1193, 2015).

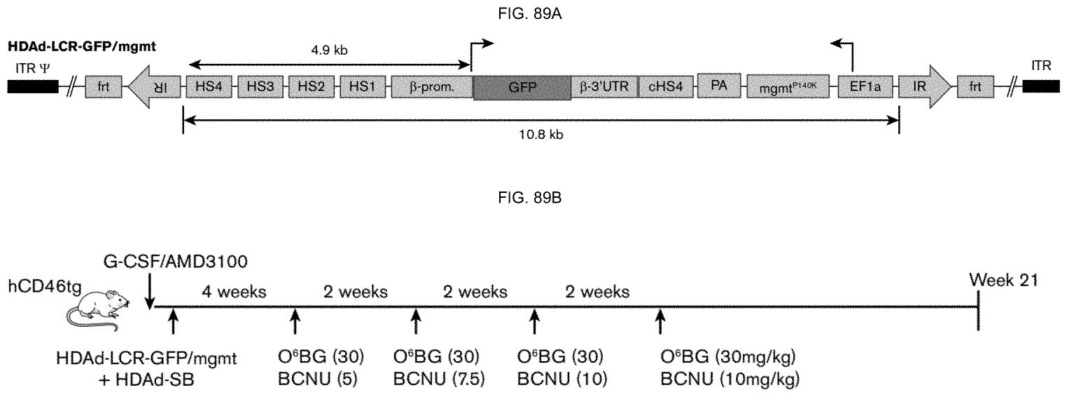

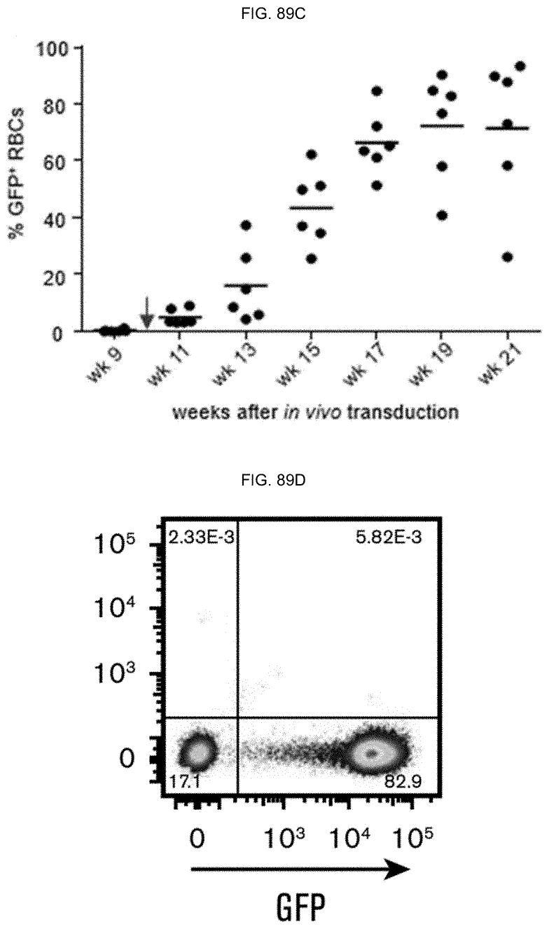

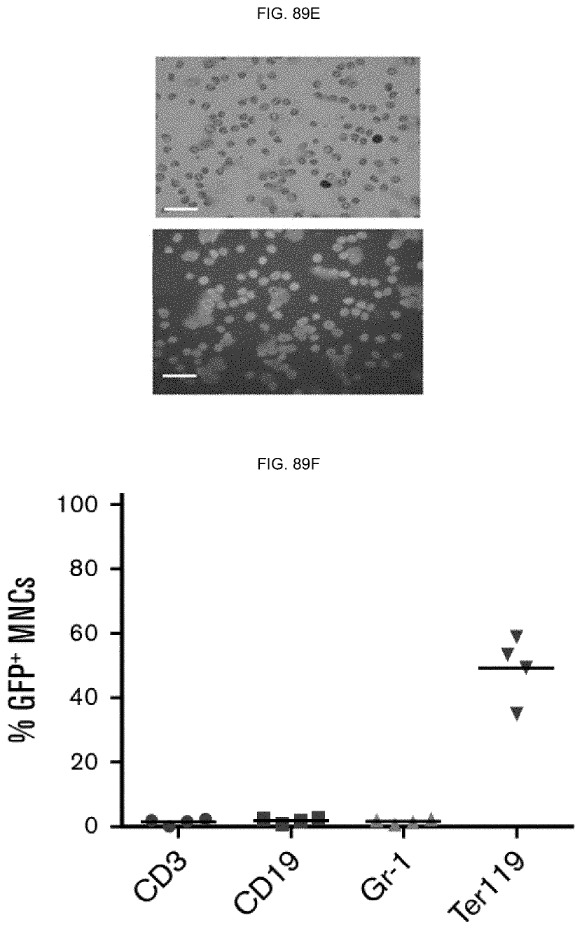

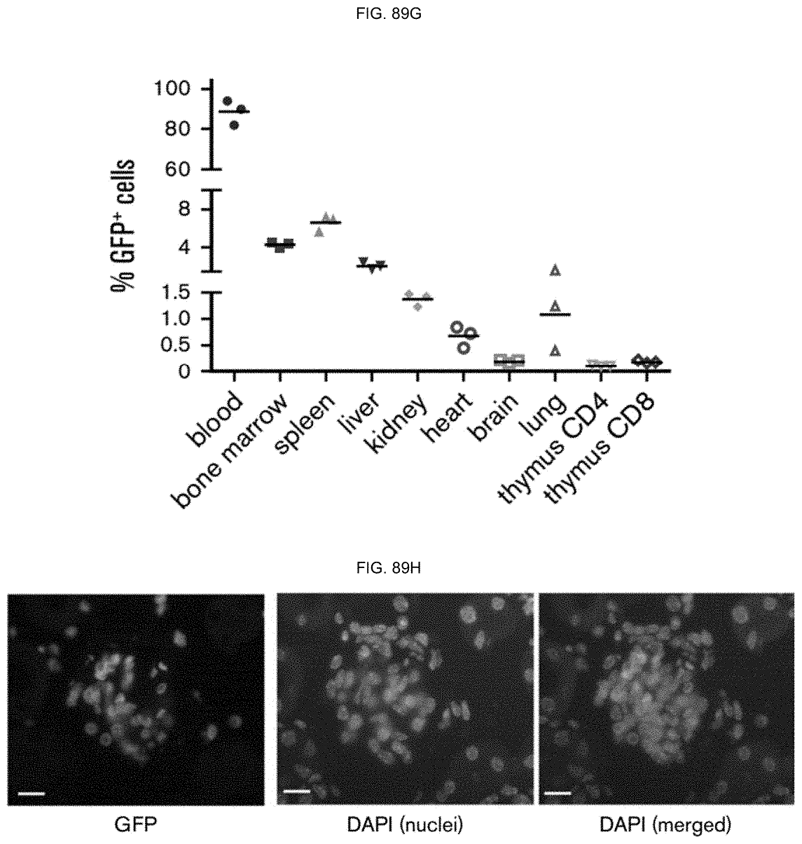

The approach to express FVIII from erythroid cells using HDAd vectors addresses these problems. This study shows, using GFP as a reporter gene under control of the mini LCR that it is possible to achieve expression of a non-erythroid protein in erythroid cells and storage of GFP in mature red blood cells after in vivo HSC transduction/selection (see FIGS. 89A-89H). It was then demonstrated in “healthy” hCD46 transgenic mice that the approach results in physiological levels of a bioengineered form of FVIII and a phenotypic correction in a hemophilia A mouse model despite the presence of anti-FVIII plasma antibodies.

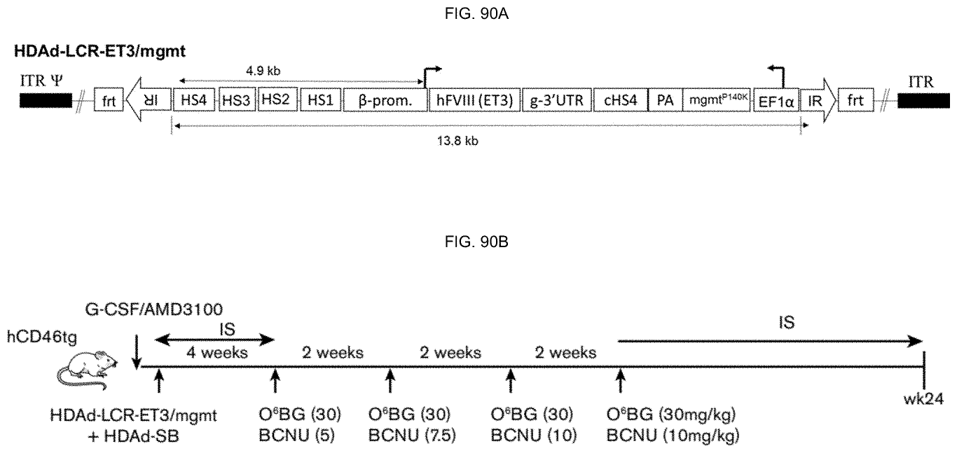

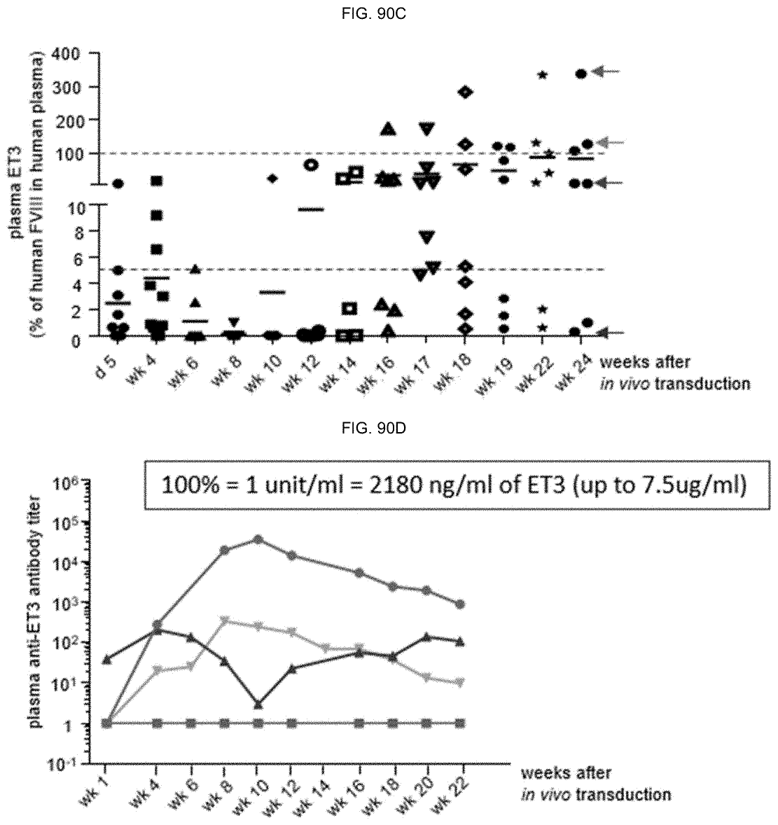

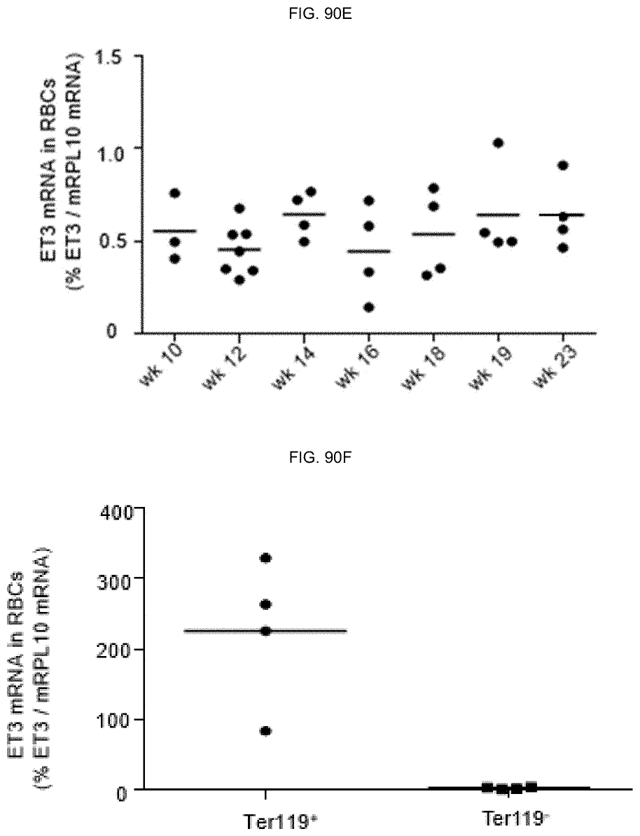

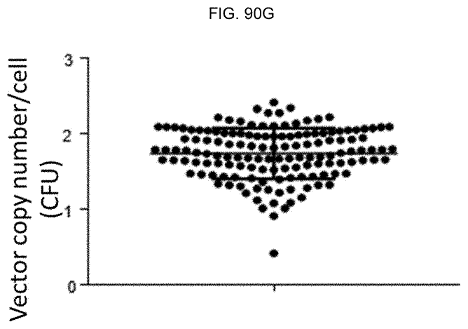

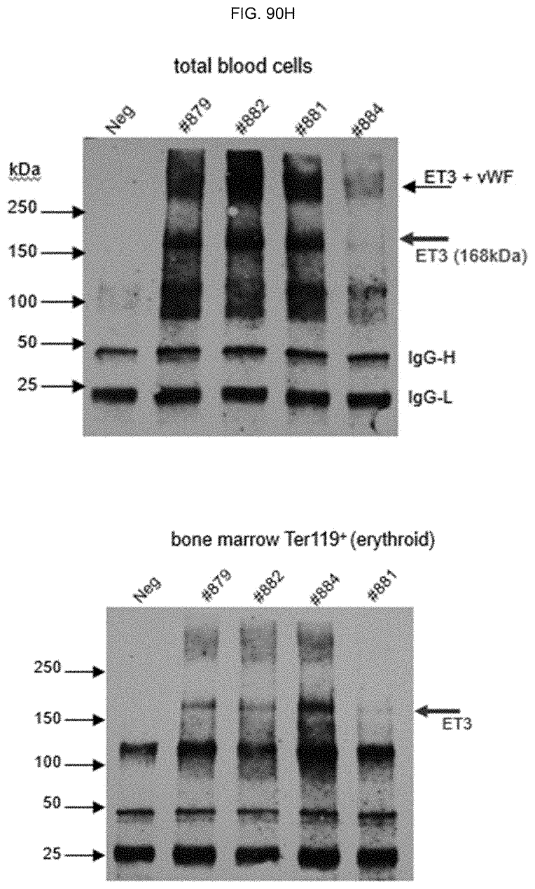

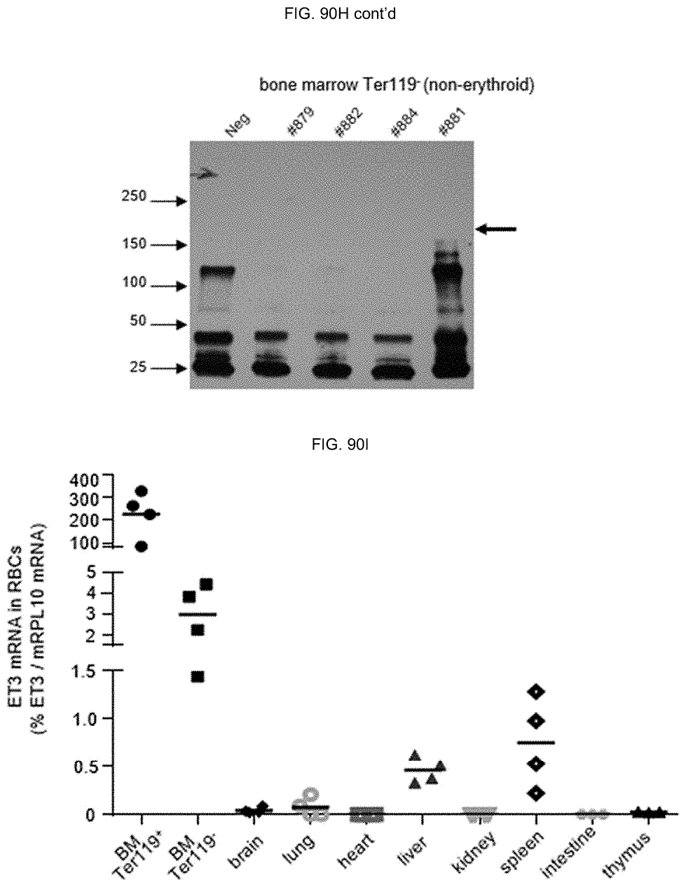

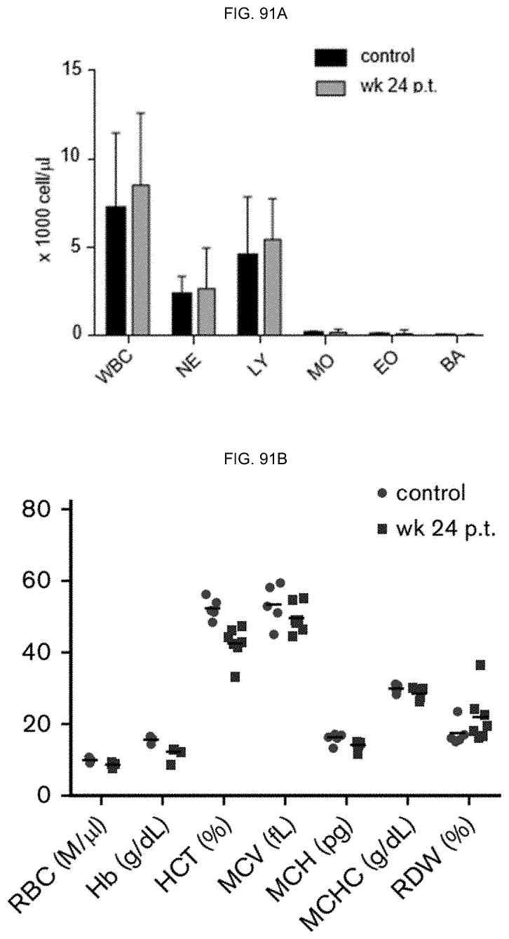

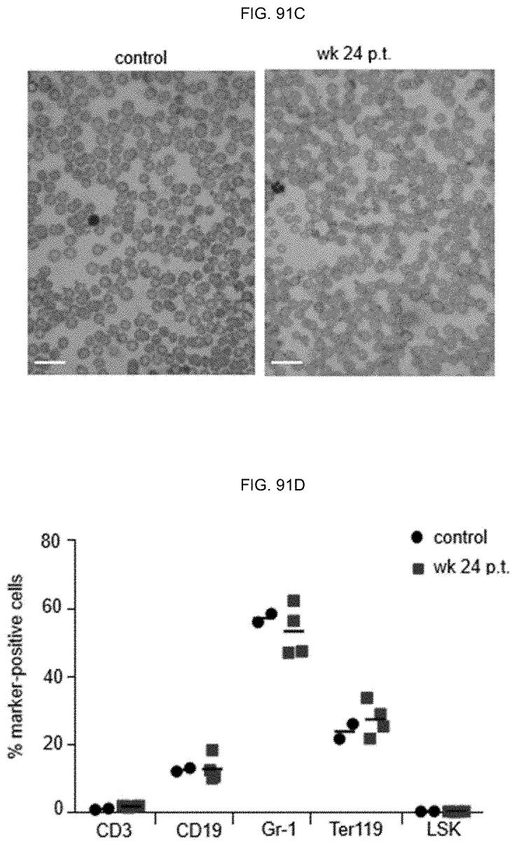

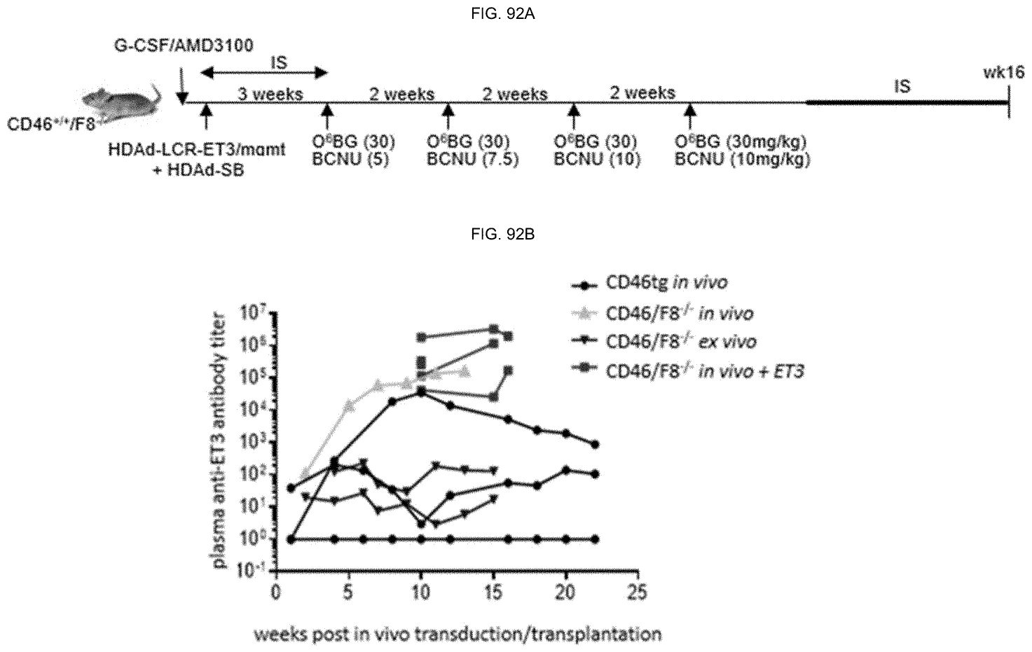

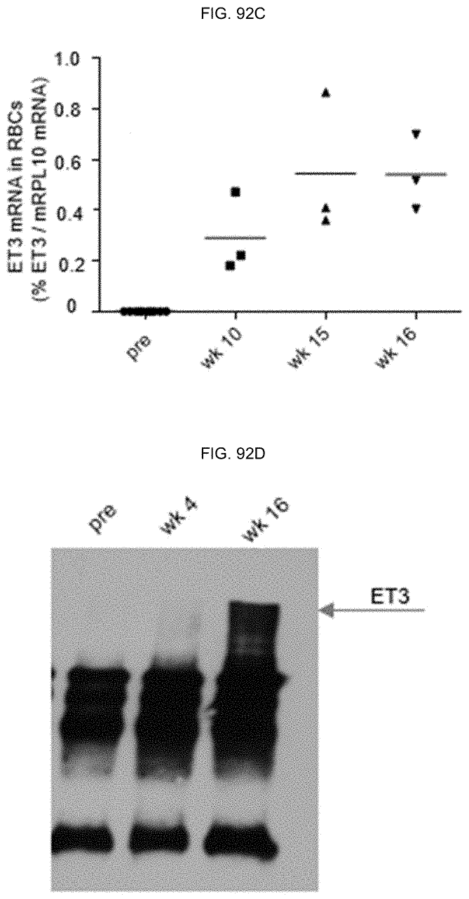

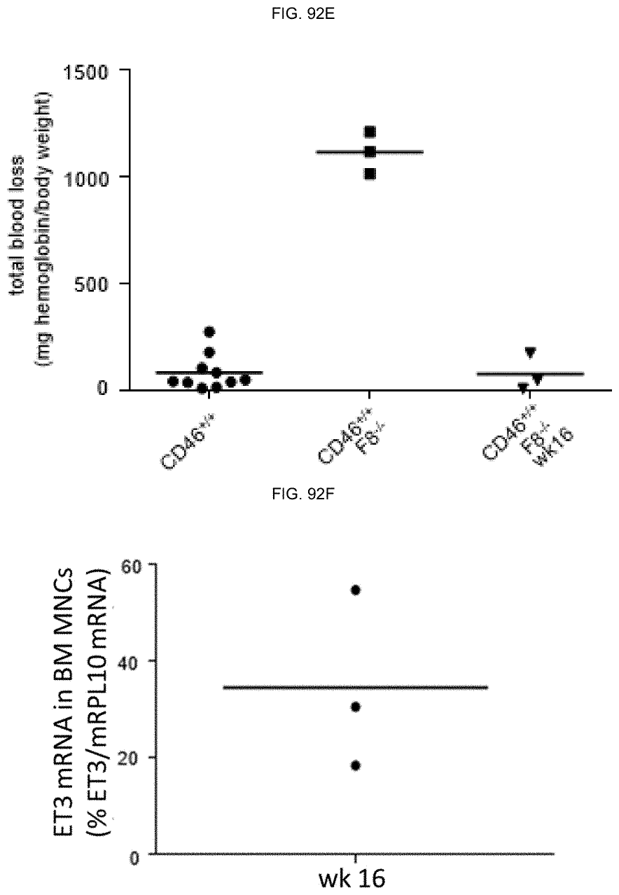

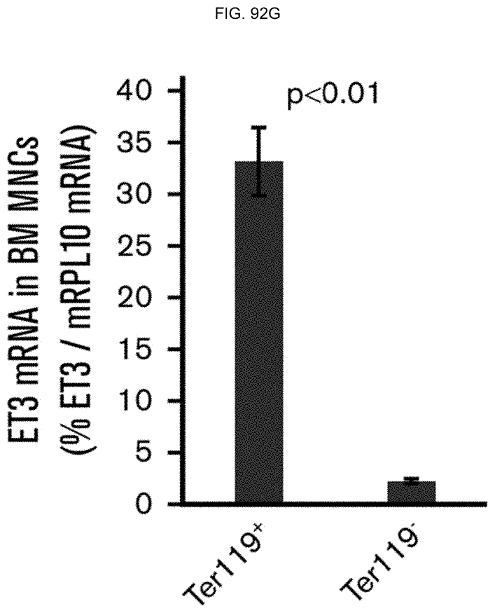

The proposed approach can provide life-long therapeutic correction after a single intravenous intervention. The enormous amplification of gene modified HSCs upon differentiation into red blood cells and the high-efficiency protein synthesis machinery of these cells create a basis for FVIII production at curative levels. Furthermore, the genetic modification of only a fraction of HSCs can result in tolerance against the transgene product. This newly developed approach for in vivo gene delivery into HSCs does not require myeloablation and HSC transplantation. It involves injections of G-CSF/AMD3100 to mobilize HSCs from the bone marrow into the peripheral blood stream and the intravenous injection of an integrating, helper-dependent adenovirus (HDAd) vector system (FIG. 90B). HDAd5/35++ and HDAd35 vectors target CD46, a receptor that is expressed on primitive HSCs. Transgene integration is achieved (in a random pattern) using a hyperactive Sleeping Beauty transposase (SB100x) (FIG. 90A). After in vivo HSC transduction/selection in CD46-transgenic mice, supraphysiological serum concentrations and activity of a bioengineered human factor VIII version (ET3) was demonstrated (FIGS. 90C-90I; 91A-91D; 92A-92G). The ET3 gene was under the control of a mini-β-globin LCR which restricted ET3 expression to erythrocytes. Despite high-level ET3 production from erythroid cells, no effects on hematopoiesis were observed. After initial development of inhibitory anti-ET3 antibody, serum antibody levels greatly decreased in 50% of treated mice most likely due to low level ET3 expression in the thymus and development of tolerance. After ex vivo and in vivo transduction of HSCs from CD46-tg/hemophilia A mice and subsequent transplantation into lethally irradiated hemophilia A mice, a phenotypic correction was achieved based on physiological factor VIII serum activity, normal aPTT, and normal bleeding time after tail clipping.

Discussion In addition to FVIII, the application of this approach for other secreted proteins can used, for example: (i) other coagulation factors, specifically FXI, FVII (Binny et al., Blood. 119(4):957-966, 2012), von Willebrand factor (VWF) (De Meyer et al., Arterioscler Thromb Vasc Biol. 28(9):1621-1626, 2008), but also rare clotting factors (i.e. factors I, II, V, X, XI, or XIII); (ii) enzymes that are currently used in Enzyme replacement therapies (ERT) for lysosomal storage diseases (taking advantage of the cross-correction mechanism) (Penati et al., J Inherit Metab Dis. 40(4):543-554, 2017) like Pompe disease (acid α-glucosidase), Gaucher disease (glucocerebrosidase), Fabry disease (α-galactosidase A), and Mucopolysaccharidosis type I (α-L-Iduronidase); (iii) immunodeficiencies e.g. SCID-ADA (Cicalese et al., Mol. Ther. 26(3):917-931 2018) (adenosine deaminase); (iv) cardiovascular diseases, e.g. familial apolipoprotein E deficiency and atherosclerosis (ApoE) (Wacker et al., Arterioscler Thromb Vasc Biol. 38(1):206-217, 2018); (v) viral infections by expression of viral decoy receptors (e.g. for HIV-soluble CD4 (Falkenhagen et al., Mol Ther Nucleic Acids. 9:132-144, 2017), or broadly neutralizing antibodies (bNAbs) for HIV (Kuhlmann et al., Mol Ther. 27(1):164-177, 2019), chronic HCV (Quadeer et al., Nat Commun. 10(1):2073, 2019), or HBV (Kuciinskaite-Kodze et al., Virus es. 211:209-221, 2016) infections; and (vi) cancer (e.g. controlled expression of monoclonal antibodies (e.g. trastuzumab (Zafir-Laviee et al, J Control Release. 291:80-89, 2018) or checkpoint inhibitors (e.g. αPDL1 (Engeland et al., Mol Ther. 22(11):1949-1959, 2014))).

### Example 8. Validation of Both the SB100x-Mediated Gene Addition and the BE-Mediated Reactivation of Endogenous γ-Globin in Non-Human Primates after In Vivo HSC Transduction

This example describes studies that will validate that both the SB100x-mediated gene addition and the BE-mediated reactivation of endogenous γ-globin are effective in non-human primates after in vivo HSC transduction.

Gene transfer vector: A gene transfer vector, HDAd-combo, will be used: The vector contains a SB100x transposase-mediated random genomic integration of the following transgenes: i) rhesus γ-globin gene under the control of a mini-LCR for efficient expression in red blood cells, rhesus mgmt^{P140K} under control of the ubiquitously active EF1a promoter for in vivo selection of transduced cells with O⁶BG/BCNU, GFP under control of the ubiquitously active EF1a promoter for analysis of peripheral blood T-cell transduction and vector biodistribution studies. It will further include adenine base editors for reactivation of endogenous γ-globin through inactivation of the BCL11a repressor protein binding sites in the HBG promoters and simultaneous inactivation of the erythroid bcl11a enhancer (which results in reduced BCL11a repressor protein expression in erythroid cells). Furthermore, the base editor expression cassette will be removed upon Flp recombinase mediated excision of the transposon resulting in only transient expression of iCas-BE. Lastly, the vector containing the SB100x transposase and Flp recombinase will not integrate and will be lost during HSC cell proliferation (FIG. 121).

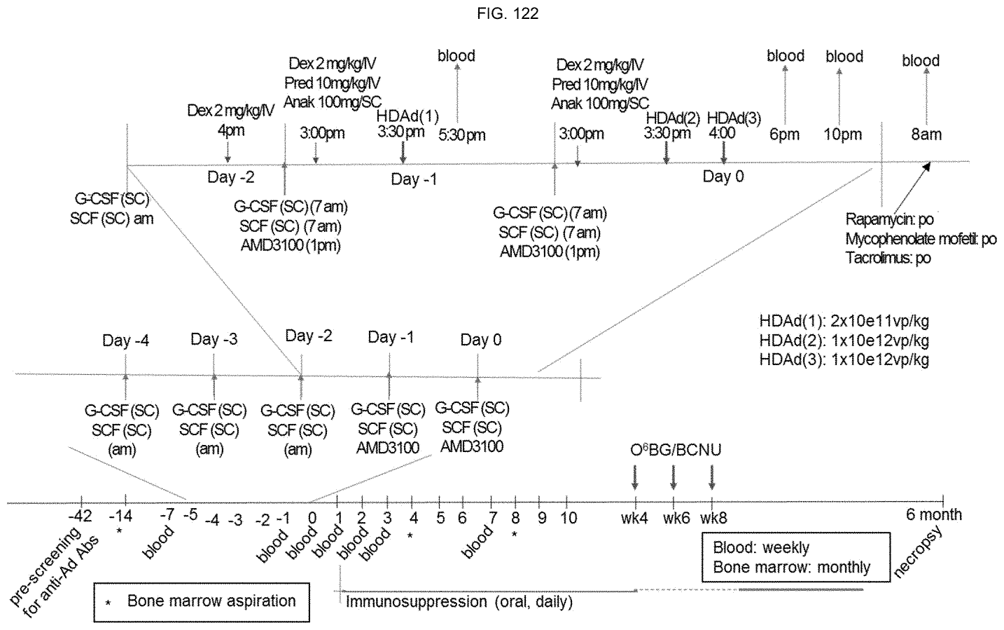

Treatment protocol: The six-months study will be performed with three Macaca mulatta using previously tested HSC mobilization and O⁶BG/BCNU in vivo selection protocols (FIG. 122). The protocol will begin with testing one animal. The study will be repeated in the remaining two animals when no serious complications occur by week 8 (end of the last in vivo selection cycle).

Mobilization: There will be 5 days of GCSF and SCF given subcutaneously in the morning (50 μg/kg each). The last two days of GCSF/SCF+AMD3100 given subcutaneously will occur in the afternoon (5 mg/kg).

Pretreatment: Dexamethasone dosed at 4 mg/kg will be given intravenously 16 hours hour before HDAd5/35++injection. Methylprednisolone dosed at 20 mg/kg plus dexamethasone dosed at 4 mg/kg will be given intravenously, while anakinra dosed at 100 mg will be given subcutaneously 30 minutes before HDAd5/35++injection.

HDAd injection: Two rounds of HDAd injections will be given intravenously: 1) a low dose (3×10¹¹ vp/kg in 20 mL of phosphate buffered saline at 2 mL/min) on daγ-1, 2) two full doses (1×10¹² vp/kg in 20 mL of phosphate buffered saline at 2 mL/min) will be given 30 minutes apart at day 0.

Transient immunosuppression: Immunosuppression will begin starting at day 1 until the first dose of O⁶BG/BCNU (week 4), and if required, continued 2 weeks after the last dose of O⁶BG/BCNU. The immunosuppression will include 0.2 mg/kg/day of rapamycin, 30 mg/kg/day of mycophenolate mofetil, and 0.25 mg/kg/day of tacrolimus, all given daily, orally via food.

In vivo selection with O⁶BG/BCNU: O⁶BG: Animals will receive 120 mg/m² O⁶BG in 200 mL of saline, intravenously infused over at least 30 mins. BCNU will be administered 60 minutes after the start of O⁶BG infusion. Animals will then receive another dose of O⁶BG in 200 mL of saline intravenously over at least 30 mins six to eight hours after BCNU administration. The first treatment will be given four weeks after HDAd injection; the second and third treatment with 2 weeks intervals (optional), depending on γ-globin marking and hematology.

Data to be collected: Blood samples will be collected as indicated in FIG. 122. Daily physical observation and weekly body weight measurements will be performed.

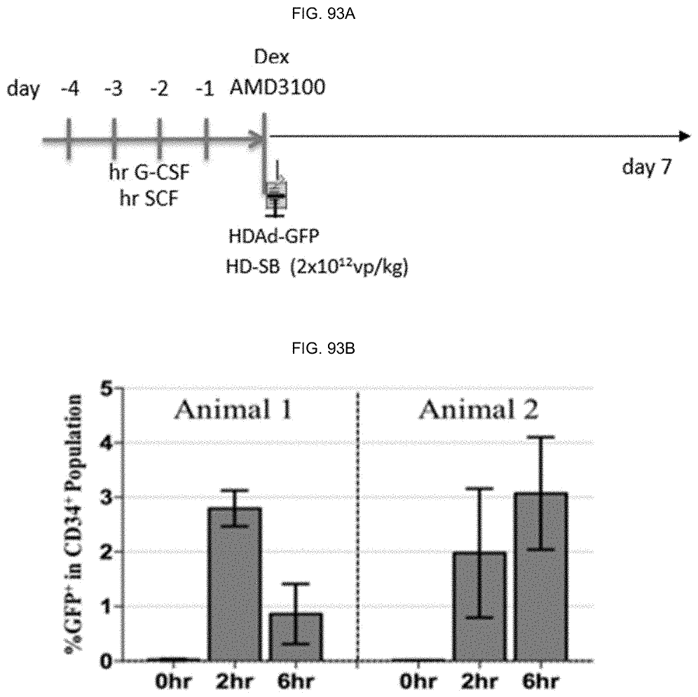

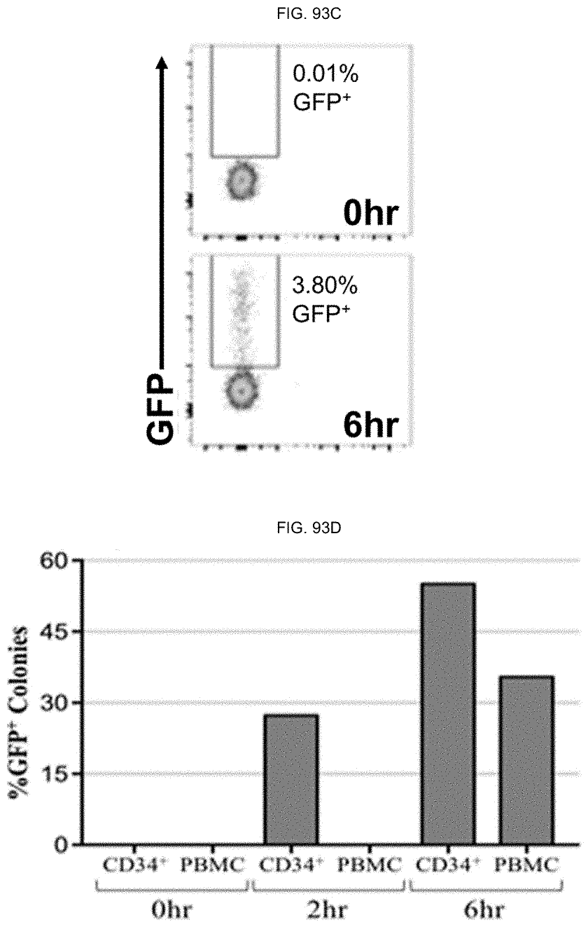

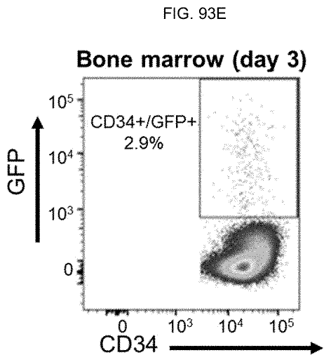

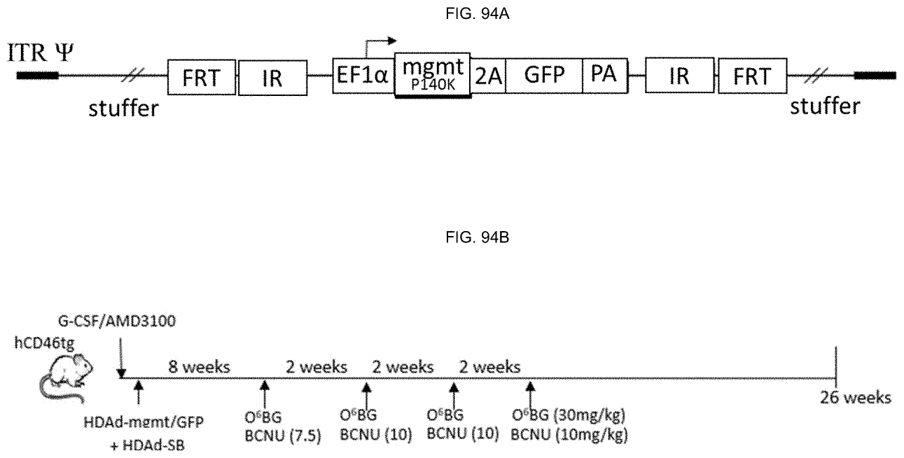

Blood samples: For two and six hour blood samples, the following assays will be performed: percentage of GFP+ cells in CD34+ and percent of GFP+ cells in CD38-/Cd45RA, CD90+ cells will be quantified, colony forming unit assays will be used to assess percent of % GFP+ colonies, migrations towards SDF1-a, and percent expression of CXCR4 and/or VLA-4 (for examples, FIGS. 93B-93E). For all other samples, blood cell counts, chemistry, c-reactive protein, and proinflammatory cytokines will be measured. γ-globin expression will be measured via flow cytometry (erythroid/non-erythroid cells), while HPLC and qRT-PCR will be used to measure levels of re-activated vs added γ-globin. Cytospins will be used to assess γ-globin immunofluorescence. Vector copy number and Cas9, SB100x, and Flpe mRNA levels will be measured. GFP expression in white blood cells (CD4+, CD8+, CD25, CD45RO, CD45RA, CCR-7, CD62L, FOXP3, integrin αeβ7) will be measured.

Bone marrow samples: Bone marrow samples will be collected on day four and then monthly (see FIG. 122). Lineage composition of bone marrow samples will be assessed by flow cytometry. Vector copy numbers in CD34+ cells will also be measured. γ-globin will be assessed using flow cytometry by sorting with Ter119+/Ter119− markers. HPLC and qRT-PCR will be used to measure levels of re-activated vs added γ-globin. In addition to these analyses, upon necropsy, whole genome sequencing will be performed on CD34+ cells to identify SB100-mediated integrations and base editor off-target effects. RNA sequencing will also be performed on CD34+ cells to compare mRNA and miRNA profiles between pre- and post-treatment.

Tissues from necropsy (including germline tissues and semen): Routine histology will be performed, and vector copy numbers will be measured on major tissue groups. γ-globin and GFP immunofluorescence will be assess on tissue sections.

Outcome: This experiment will validate that both the SB100x-mediated gene addition and the BE-mediated reactivation of endogenous γ-globin are effective in non-human primates after in vivo HSC transduction. It will demonstrate that the vector will achieve γ-globin expression levels in red blood cells that would be curative in SCA patients (i.e. >80% γ-globin⁺ RBCs with γ-globin levels >20% of adult rhesus globin). It will also demonstrate an absence of long-term hematological side-effects and absence of undesired genomic rearrangements and changes in the transcriptome of HSCs. Lastly, it will demonstrate that intravenously injected HDAd5/35++ vector transduces memory T-cells.

### Example 9. Human and Rhesus Macaque HSC Transduction with HDAd5/35++ Vectors Expressing Base Editors for Re-Activation of Endogenous γ-Globin Bin Expression

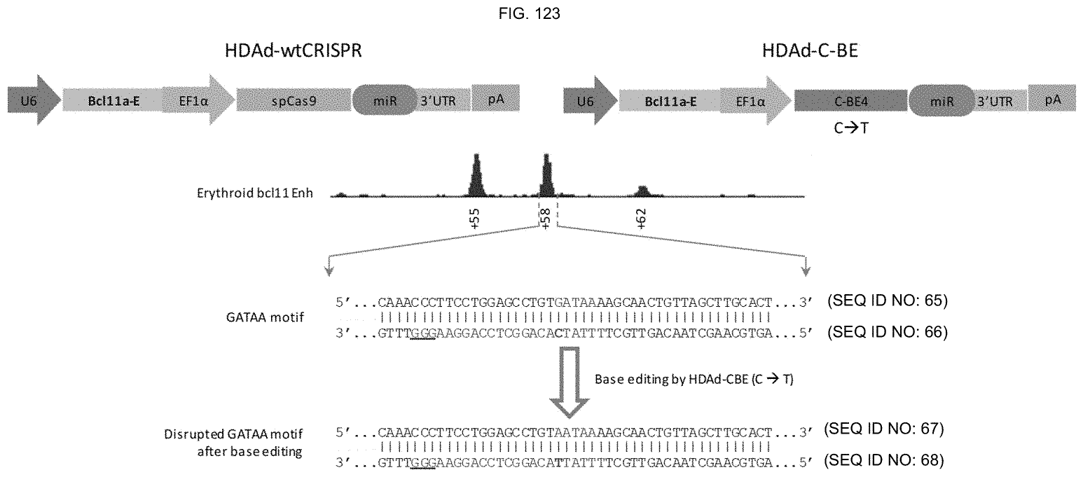

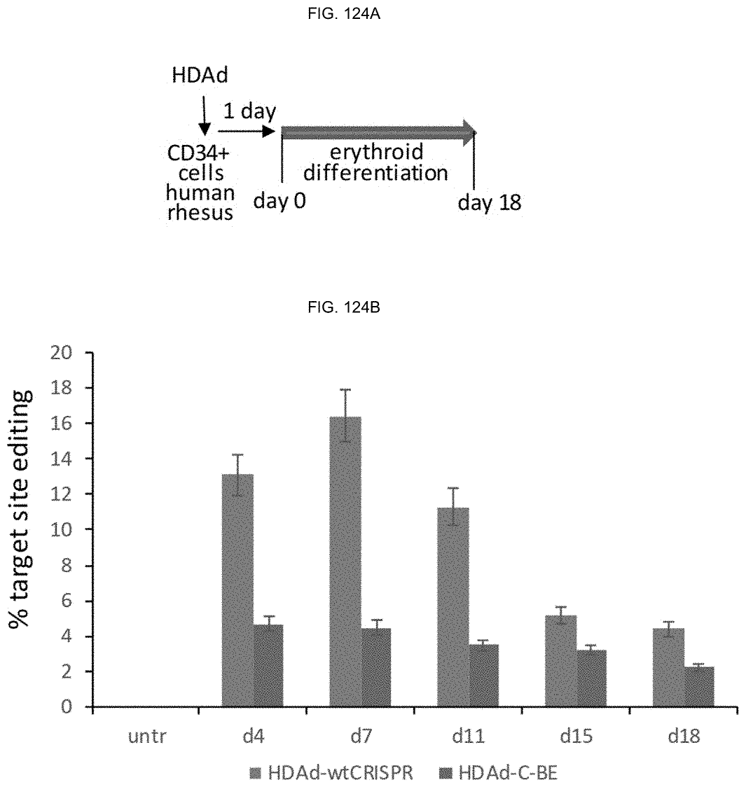

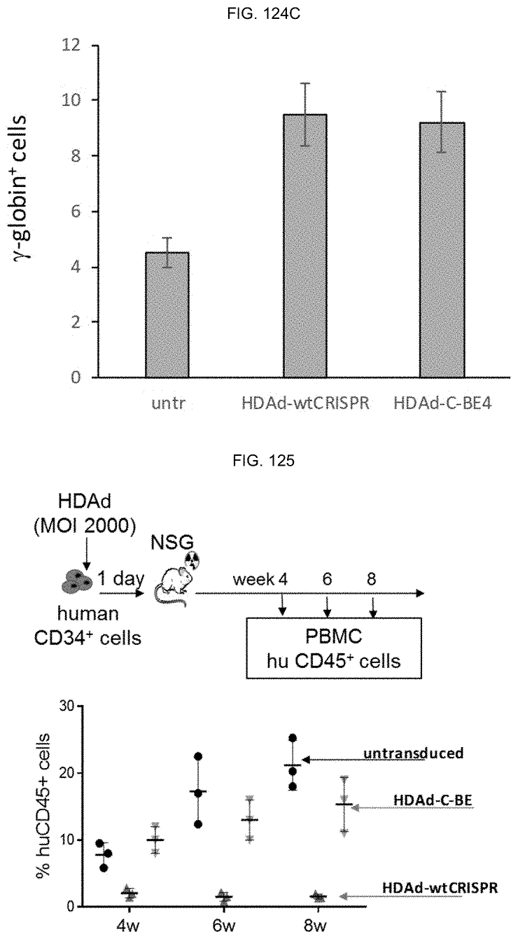

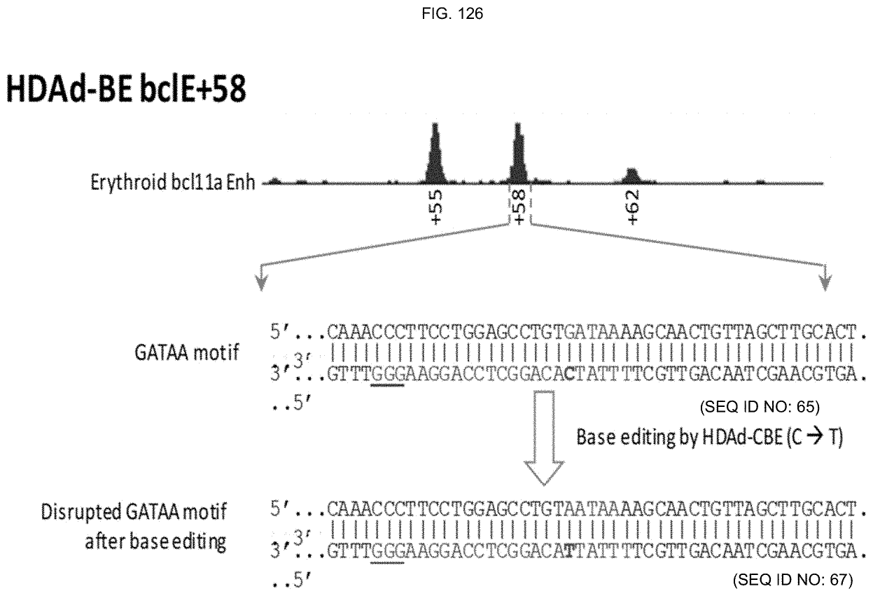

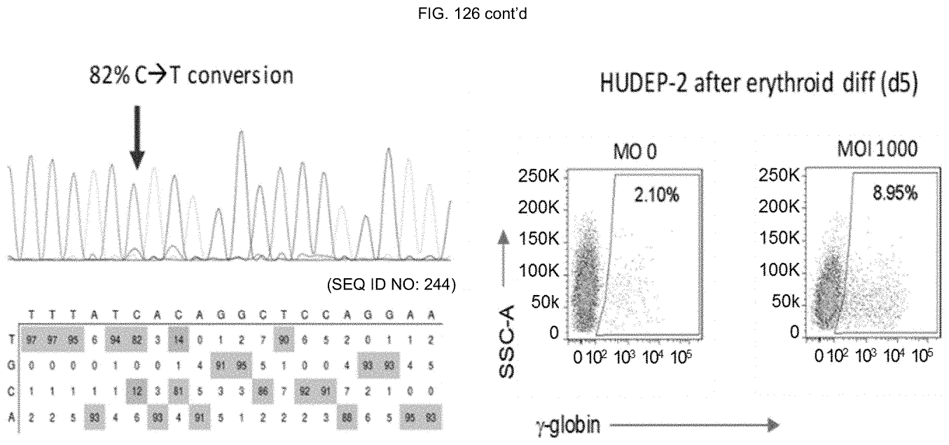

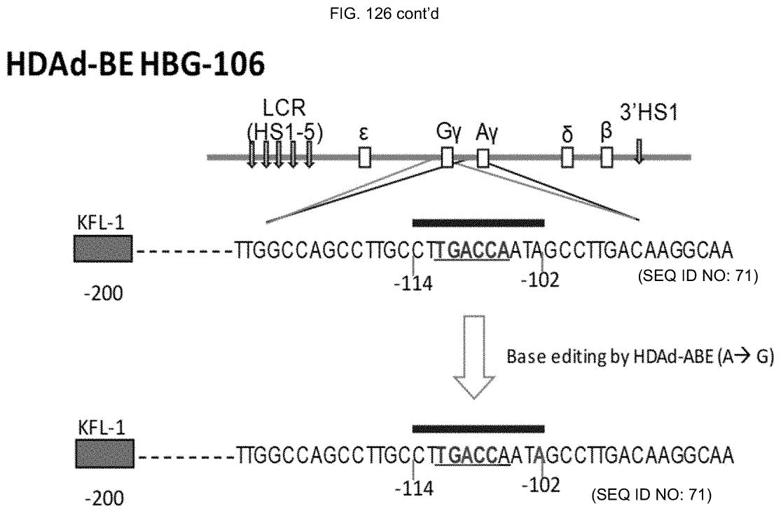

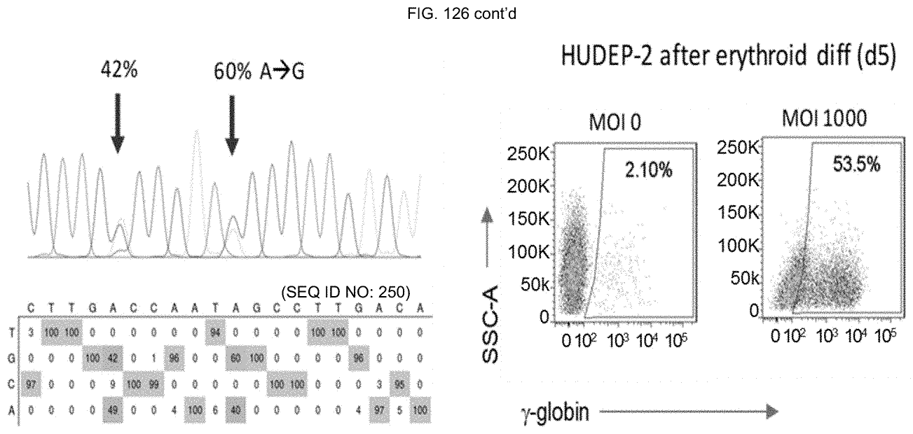

Inactive Cas9 fused to a either a cytidine or adenine deaminase or transaminase may serve as tools to reactivate fetal globin. An HDAd vector expressing the cytidine base editor (HDAd-C-BE) was compared with a HDAd-CRISPR/Cas9 vector targeting the erythroid bcl11a enhancer and destroying a critical GATA binding motif (FIG. 123). An HDAd vector expressing a wild-type CRISPR against the same region was constructed. Both vectors were tested on human CD34+ cells that, after HDAd transduction, were subjected to erythroid differentiation over 18 days (FIG. 124A). For HDAd-wtCRISPR transduced cells, a gradual decline in the percentage of edited target sites was observed, most likely due to CRISPR-related cytotoxicity (FIG. 124B). While the efficacy of genome editing was lower for HDAd-C-BE vector, the editing rate remained stable, resulting in comparable reactivation of γ-globin (FIG. 124C). After transplantation, engraftment of HDAd-C-BE transduced CD34+ cells was as efficient as that of untransduced control cells (FIG. 125). In summary, these data indicate that base-editor vectors are, potentially, a better tool for genome editing in HSCs than wtCRISPR-expressing vectors. More recently, a series for HDAd vectors expressing adenine editors against three different regions in the HBG1/2 promoters were developed. It is expected that γ-globin reactivation can be substantially increased by simultaneously targeting several repressor levels with base editor vectors. Toward this goal HDAd vectors expressing base editors targeting the erythroid bcl11a enhancer (FIG. 126, upper panel) or the BCL11a protein binding site in the HBG1/2 were tested (FIG. 126, lower panel). γ-globin reactivation in an in vitro study was 9 and 53% for the two vectors, respectively.

Data in the SCA mouse model (Townes model): B6; 129-Hbb^{tm2(HBT1,HBB*)Tow}/Hbb^{tm3(HBG1,HBB)Tow}/^{Hbatm1(HBA)Tow}/J; hα/hα::β^{A}β^{S}, hα/hα::383 γ-β^{A}/-1400 γ-β^{S}.

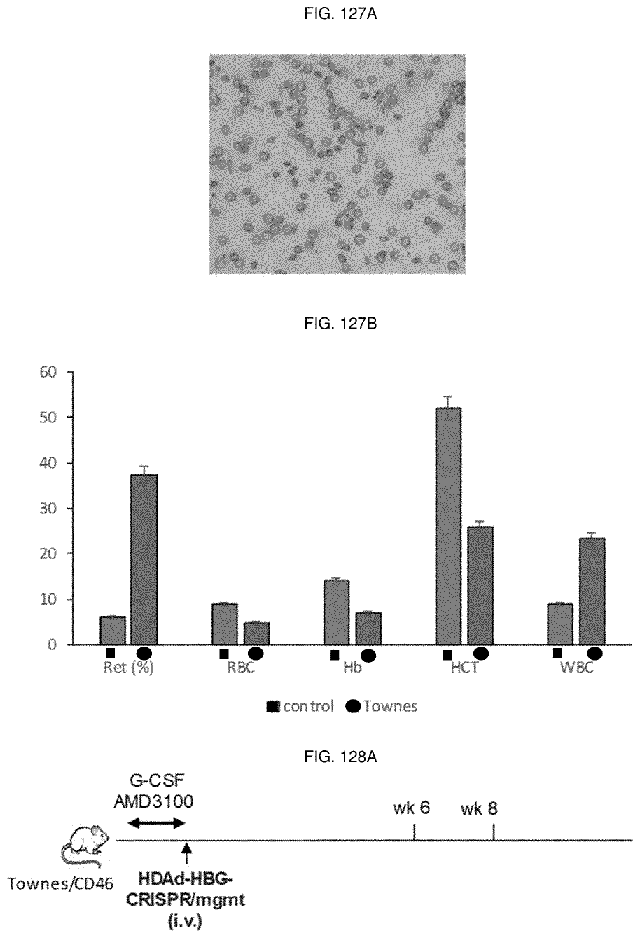

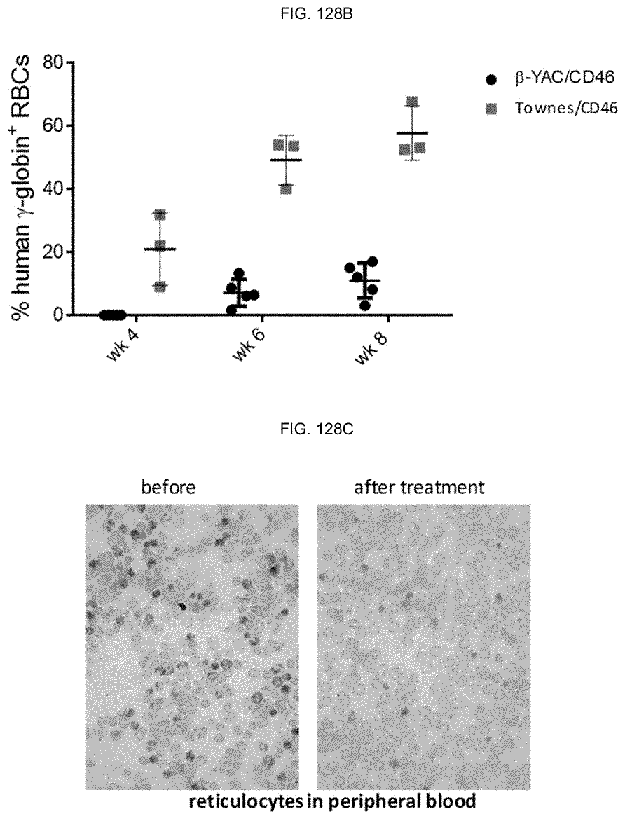

The mice contain human α-globin, γ-globin (including -383 and -1400 regions containing the promoters), β⁸⁷-SCA globin instead of corresponding mouse genes and show a severe SCA phenotype (FIG. 127A) with 40% of reticulocytes in peripheral blood, low hematocrit, low hemoglobin levels, and leukocytosis (FIG. 127B). These mice were bred to achieve homozygosity for CD46 and the three globin gene substitution (CD46/Townes mice). It was tested to determine whether the previously developed HDAd-HBG-CRISPR vector would activate γ-globin after in vivo HSC transduction of CD46/Townes mice (FIG. 128A). Without O⁶BG/BCNU selection, γ-globin marking of RBCs reached 60%, indicating that the functional deficiency in erythropoiesis of Townes mice provides a strong proliferation stimulus for genome-edited HSCs/erythroid progenitor cells (FIG. 128B). The therapeutic effect of the HDAd-HBG-CRISPR vector was reflected in a greatly improved erythrocyte phenotype and an 5-fold reduction in peripheral reticulocytes (FIG. 128C). This indicates that a cure in this model (and potentially in SCA patients) could be achieved without the need of O⁶BG/BCNU in vivo HSC selection.

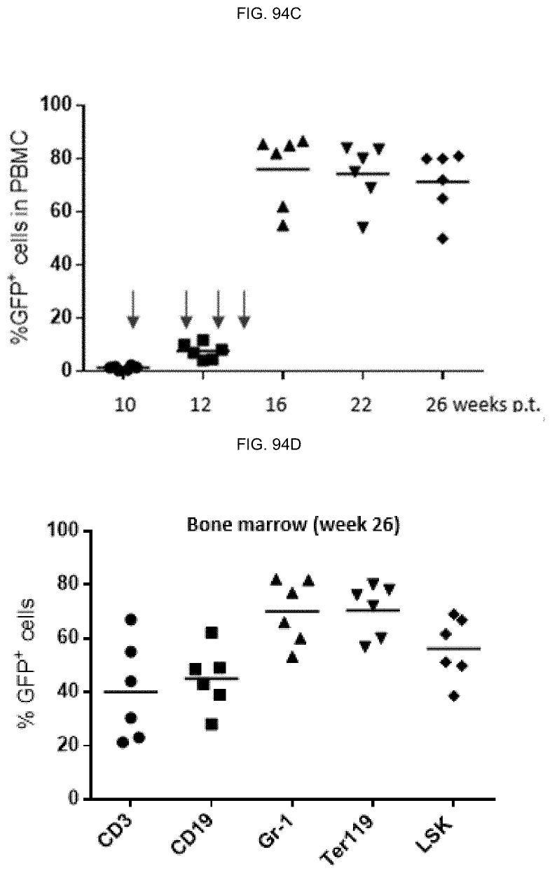

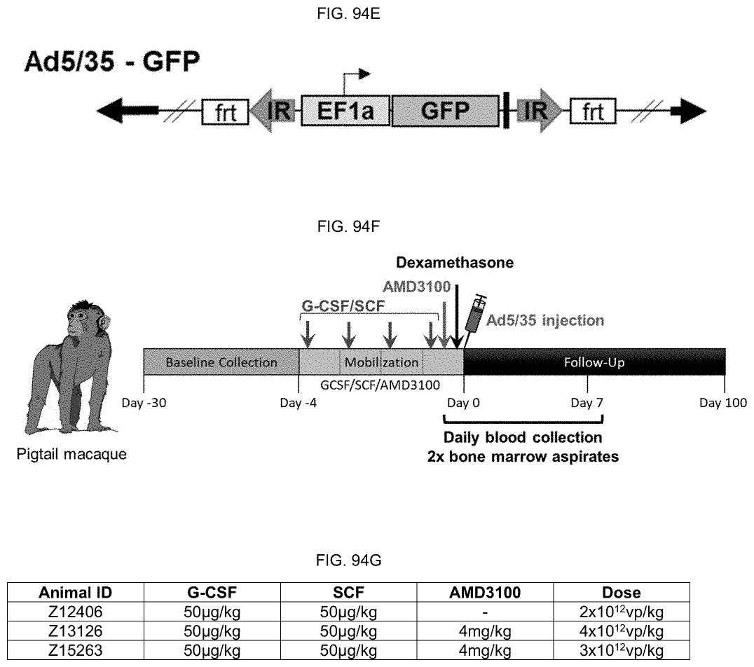

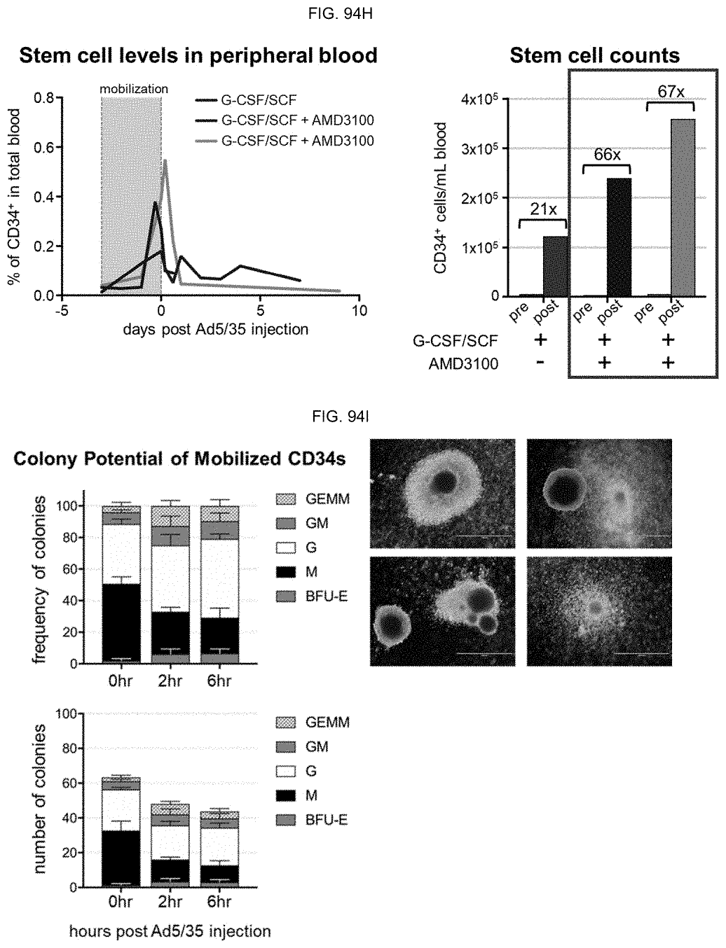

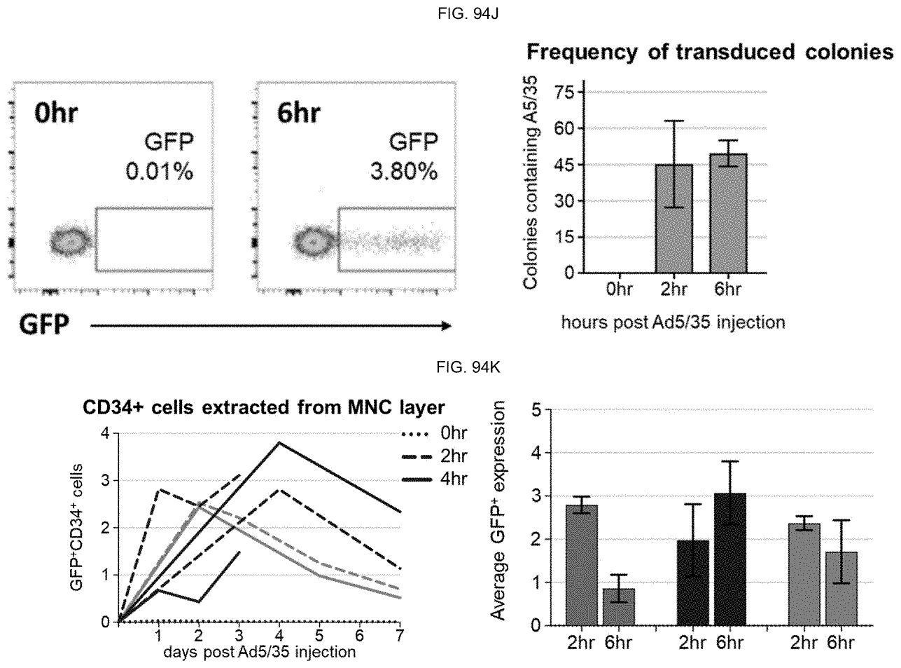

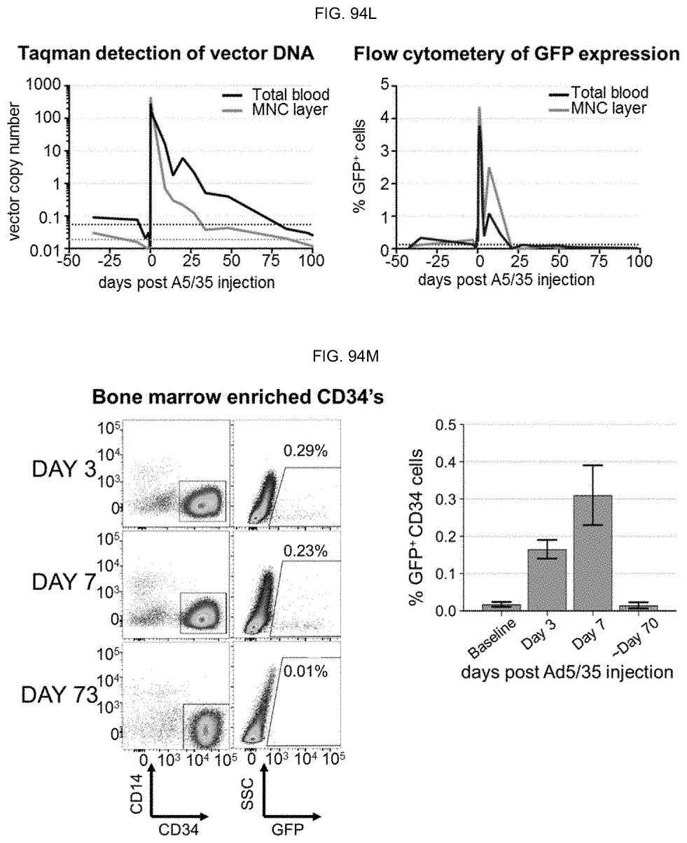

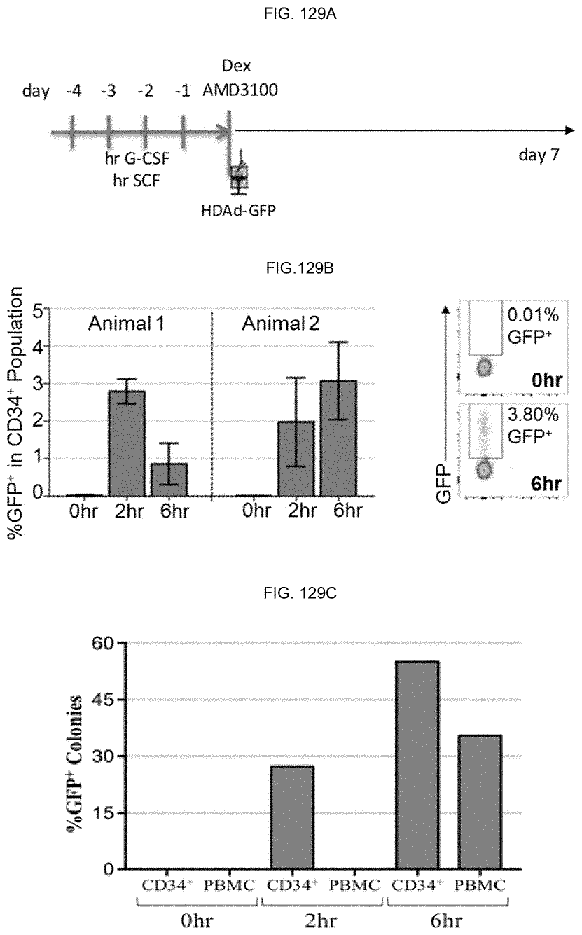

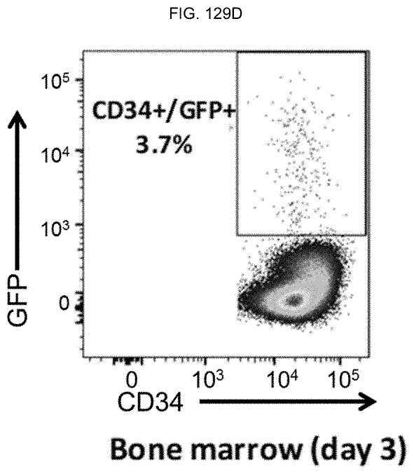

In vivo HSC gene transfer in non-human primates (NHPs): These data are from two NHPs (Macaca nemestrina) that received mobilization with G-CSF, SCF, and AMD3100 followed by injection of HDAd-GFP (FIG. 129A; FIG. 93A; FIGS. 94E-94G). Peripheral blood samples were collected immediately before vector injection, and 2 and 6 hours post vector injection. Isolated CD34+ cells were cultured ex vivo and plated in colony forming assays. An average of 3% of the CD34+ cells isolated following vector administration were GFP+ (FIG. 129B; FIGS. 93B, 93C; FIG. 94H), suggesting that mobilized CD34+ cells in peripheral blood can be transduced by a single intravenous administration of a HDAd5/35++ vector. To test whether these CD34+ cells retained colony-forming potential, colony assays were performed, and determined the percentage of colonies that carried the GFP transgene via PCR. Up to 55% of colonies derived from CD34+ cells from the post-injection time point were transduced by the vector (FIG. 129C; FIG. 93D; see also FIGS. 941-94M). Finally, to test the ability of in vivo vector-targeted cells to home back to the bone marrow compartment following peripheral mobilization, bone marrow aspirates were collected from one of the animals 3 days post vector administration. 3.7% or 2.9% of bone marrow-resident CD34+ cells were GFP+, and no appreciable difference in colony forming potential was observed in cells collected before vs. after in vivo delivery (FIG. 129D; FIG. 93E). These non-human primate studies (performed with a vector dose that was 10x lower than in mice) demonstrate that the described in vivo delivery approach is feasible and safe in a validated preclinical model.

### Example 10. In Vivo HSC Gene Therapy with Base Editors Allows for Efficient Reactivation of Fetal γ-Globin in β-YAC Mice

This example demonstrates that base editors delivered by HDAd5/35++ vectors in vivo are a useful and effective strategy for precise genome engineering, e.g., for the treatment of hemoglobinopathies.

Base editors are capable of installing precise nucleotide mutations at targeted genomic loci and present the advantage of avoiding double-stranded DNA breaks. Here, critical motifs were targeted regulating γ-globin reactivation with base editors delivered via HDAd5/35++ vectors. Through optimized design, a panel of cytidine and adenine base editors (CBE and ABE) targeting the BCL11A enhancer or recreating naturally occurring Hereditary Persistence of Fetal Hemoglobin (HPFH) mutations in the HBG1/2 promoter were successfully rescued. In HUDEP-2 cells, all five tested vectors efficiently installed target base conversion and led to substantially-globin reactivation. Significant γ-globin protein production (23% over β-globin) was observed by using an ABE vector HDAd-ABE-sgHBG #2 specific to the -113A to G HPFH mutation in HBG1/2 promoter. This vector was therefore chosen for downstream animal studies. Mice that carry a 248 kb of human β-globin locus (β-YAC mice) were used and thus accurately reflect globin switching. An EF1α-MGMT^{P140K} expression cassette flanked by FRT and transposon sites was included in the vector for allowing in vivo selection of transduced cells. After in vivo transduction with HDAd-ABE-HBG #2+HDAd-SB and low doses of chemoselection, an average of over 40% HbF-positive cells in peripheral red blood cells was measured. This corresponded to 21% γ-globin production over human β-globin. The -113 A to G conversion in total bone marrow cells was on average 20%. Compared to untransduced mice, no alterations in hematological parameters, erythropoiesis and bone marrow cellular composition were observed after treatment, demonstrating a good safety profile of the approach. No detectable editing was found at top-scored potential off-target genomic sites. Bone marrow lineage minus cells were isolated from primary mice at week 16 after transduction and infused into lethally irradiated C57BL/6J mice. The percentage of HbF-positive cells was maintained in secondary recipients over 16 weeks indicating genome editing occurred in long-term repopulating mouse HSCs. The observations demonstrate that base editors delivered by HDAd5/35++ vectors represent a promising strategy for precise in vivo genome engineering for the treatment of hemoglobinopathies.

Genome engineering strategies based on nucleases such as CRISPR/Cas9 have achieved remarkable advances, with multiple gene therapy studies having entered the phase of clinical evaluation. The CRISPR/Cas9-mediated gene editing relies on double-stranded DNA breaks (DSBs) that trigger endogenous repair mechanisms including classical non-homologous end joining (NHEJ). In the presence of a donor DNA template, homology-directed repair (HDR) can occur at a typically lower frequency. Latest studies have demonstrated highly efficient disruption of gene of interest in hematopoietic stem and progenitor cells (HSPCs) that are important for genetic therapy for blood disorders (Martin et al., Cell Stem Cell 24: 821-828.e825, 2019; Wu et al., Nature Medicine 25: 776-783, 2019). However, studies have reported that nuclease-induced DSBs may (Haapaniemi et al., Nature Medicine, 24(7):927-903, 2018; Ihry et al., Nature Medicine, 24(7):939-946, 2018; Kosicki et al., Nature Biotechnology 36: 765, 2018) cause side effects to host cells by generating unwanted large fragment deletion and p53-dependent DNA damage responses (Haapaniemi et al., Nature Medicine, 24(7):927-903, 2018; Ihry et al., Nature Medicine, 24(7):939-946, 2018; Kosicki et al., Nature Biotechnology 36: 765, 2018).

Base editors (BEs) are capable of installing precise nucleotide substitutions at targeted genomic loci without creating DSBs. They include a catalytically disabled nuclease, such as Cas9 nickase (nCas9) that is incapable of making DSBs, fused to a nucleobase deaminase enzyme and, in some cases, a DNA glycosylase inhibitor. Currently, there are two major categories, cytidine base editors (CBEs) and adenine base editors (ABEs), which convert C >T and A >G transitions, respectively, in a narrow targetable window (usually around 5 base pairs) dictated by a single guide RNA (sgRNA) coupled with the nCas9 (Gaudelli et al., Nature 551: 464-471, 2017; Komor et al., Nature 533: 420-424, 2016; Nishida et al., Science 353, 2016). The key difference between CBEs and ABEs is located in the deaminase region where CBEs contain a cytidine deaminase (e.g., APOBECI) and ABEs use laboratory-evolved TadA deoxyadenosine deaminases. Multiple groups have reported efficient base editing in a variety of eukaryotic cells (Zhang et al., Genome Biology 20: 101, 2019; Chadwick et al., Arterioscler Thromb Vasc Biol 37: 1741-1747, 2017; Zeng et al., Nature Medicine 26: 535-541, 2020; Lim et al., Mol Ther, 82(4):1177-1189, 2020; Gao et al., Nature 553: 217-221, 2018). It is predicted that 60% of all known pathogenic single nucleotide polymorphisms (SNPs) in humans can be potentially reversed by current BEs (Rees et al., Nature Reviews Genetics 19: 770-788, 2018).

β-hemoglobinopathies is a common group of genetic disorders with absent or deficient production or normal β-globin—mainly including β-thalassemia and sickle cell disease (SCD). Depending on the specific genetic defects, β-thalassemia and SCD patients exhibit various severity of disease manifestations. Although with newborn screening and treatment prophylaxis the mortality in SCD children has largely decreased, most β-thalassemia major (β⁰) and SCD patients suffer from lifelong acute and chronic complications (Ware et al., Lancet 390: 311-323, 2017; Higgs et al., Lancet 379: 373-383, 2012). However, in some adult patients with high level fetal hemoglobin (HbF), which predominates during much of gestation stage and is normally silenced shortly after birth, the disease symptoms are markedly milder. This phenomenon of hereditary persistence of fetal hemoglobin (HPFH) demonstrate a strong protective effect of HbF and provide a good rationale for reactivation of γ-globin as a gene therapy strategy for patients with β-globin disorders.

A number of HPFH mutations have been reported (reviewed by Orkin & Bauer, Annual Review of Medicine 70: 257-271, 2019 and Wienert et al., Trends in Genetics: TIG 34: 927-940, 2018). There are three major clusters of HPFH SNPs located at around -150, -175 and -200 sites in the HBG1/2 promoter. Introduction of HPFH mutations at these sites can disrupt the binding sites of HbF repressors (e.g., BCL11A and ZBTB7A) or create gain-of-function binding sites for activators (e.g., TAL1 and KLF1), leading to derepressed HbF expression (Traxler et al., Nature Medicine 22: 987-990, 2016; Martyn et al., Nature Genetics 50: 498-503, 2018). HbF reactivation can also be achieved by modulating the expression of HbF regulators, such as BCL11A, a major HbF repressor (Sankaran et al., Science 322: 1839-1842, 2008). Although direct BCL11A knock-out is not optional due to its developmentally indispensable roles, partial downregulation of BCL11A by editing its erythroid-specific enhancers allow for efficient HbF induction while maintaining animal viability (Wu et al., Nature Medicine 25: 776-783, 2019; Canver et al., Nature 527: 192-197, 2015). Using BE:sgRNA ribonucleoprotein (RNP) electroporation, a recent study has demonstrated that disruption of critical motifs in the +58 BCL11A enhancer with base editors leads to therapeutic HbF induction in patient-derived CD34⁺ HSPCs.

A simplified gene therapy approach has been recently established by in vivo HSC transduction. Help-dependent HDAd5/35++ vectors were used due to their multiple advantageous properties including chimeric fiber for HSC tropism, over 32 kb payload to accommodate most commonly used transgenes, etc. In this study, using optimized design a panel of BE vectors was successfully generated targeting the BCL11A enhancer or HBG1/2 promoter. In a transgenic mice model, it is shown here that in vivo HSC base editing with an HDAD-ABE vector recreated HPFH mutation and led to efficient HbF induction.

Materials and Methods.

Reagents for in vivo transduction and selection: G-CSF (Neupogen™) (Amgen, Thousand Oaks, Calif.), AMD3100 (MilliporeSigma, Burlington, Mass.) and Dexamethasone Sodium Phosphate (Fresenius Kabi USA, Lake Zurich, Ill.) were used. O⁶-Benzylguanine (O⁶-BG) and Carmustine (BCNU) were from MilliporeSigma.

Generation of HDAd vectors: Base editing systems developed by David R. Liu's lab at Harvard were used (Koblan et al., Nature Biotechnology 36: 843-846, 2018). pCMV_AncBE4max and pCMV_ABEmax plasmids were purchased from Addegene (Watertown, Mass.). The following plasmids from Addgene were also used: BE4, ABE7.10, pLenti-BE3RA-PGK-Puro and pLenti-FNLS-PGK-Puro and BE3RA in FIGS. 131A & 131B (Zafra et al., Nature Biotechnology 36: 888-893, 2018). Oligos and gBlocks described below were synthesized by Integrated DNA Technologies (IDT) (Coralville, Iowa) and listed in Table 14.

TABLE 14
Guide sequences for base editors.

| Editor | Name | Sequence (5′ to 3′)* | Targeting site/note |
|---|---|---|---|
| BCL11A | | | |
| CBE | sgBCL#1 | TTTAT [symbol] ACAGGCTCCAGGAA | GATAA motif |
| CBE | sgBCL#2 | TTTTAT [symbol] ACAGGCTCCAGGA | GATAA motif |
| ABE | sgBCL#3 | TTT [symbol] TCACAGGCTCCAGGAA | GATAA motif |
| ABE | sgBCL#4 | TTTT [symbol] TCACAGGCTCCAGGA | GATAA motif |
| ABE-xCas9 | sgBCL#5 | CTGTGATAAAAGCAACTGTT | GATAA motif, NGC PAM |
| ABE-xCas9 | sgBCL#6 | GATAAAAGCAACTGTTAGCT | GATAA motif, NGC PAM |
| HBG | | | |
| CBE | sgHBG#1 | CTTGA [symbol] AATAGCCTTGACA | TGACCA, −114−115 CC > GG |
| ABE | sgHBG#2 | CTTGACC [symbol] ATAGCCTTGACA | TGACCA, −113 A > G |
| ABE | sgHBG#3 | GCT [symbol] TTGGTCAAGGCAAGGC | −111 T > C |
| ABE | sgHBG#4 | GTGGGGAAGGGGCCCCCAAG | −198 T > C |
| CBE-xCas9 | sgHBG#5 | CCTTCCCCACACTATCTCAA | −197 C > T, −196 C > T, NGC PAM |
| ABE-xCas9 | sgHBG#6 | AGATATTTGCATTGAGATAG | −175 T > C, NGT PAM |
| HBB | | | |
| CBE | sgHBB_STOP | CTTGCC [symbol] CAGGGCAGTAA | HBB CDS, TGG > TAA (HBB STOP) |
| ABE | sgHBB_SKIP | AGACTCACCCTGAAGTTCTC | HBB intron, GT > GC (HBB splicing SKIP) |
| Positive control | | | |
| CRISPR/Cas9 | sgHBG_CRISPR | CTTGTCAAGGCTATTGGTCA | TGACCA |
| Negative controls | | | |
| CBE | sgNeg | GGTGTCGAAATGAGAAGAAG | CCR5 CDS, nt673 C > T (CCR5 STOP) |

Underlined: targeting base(s) in critical motifs.
*From top to bottom: SEQ ID NOs: 244, 245, 244, 245, 248-250, 250, 252-259.

CBE and first version of ABE constructs: The cloning involved 3 steps. Step 1) The BsmBI site in BE4 was destroyed by replacing the Eagl-Nael fragment with gBlock #1. The BsmBI site in pCMV_AncBE4max was destroyed by replacing the BsmBI-Narl fragment with gBlock #2. A vector named pBST-CRISPR with a BsmBI sgRNA cloning site was generated by combining the following four fragments using infusion (Takara, Mountain View, Calif.): a 2.3 kb U6-filler-gRNA scaffold fragment amplified from LentiCRISPRv2 (Addgene) using #3FR, a 1.4 k b and 1.0 kb fragments amplified from pBST-sgBCL11Ae1 (Li et al., Blood 131: 2915-2928, 2018) using #4FR and #5FR, respectively, and a 9.6 kb fragment of pBST-sgBCL11Ae1 released by Bsal-BamHI digestion. An intermediate plasmid pBS-U6-Ef1α was composed by joining the following three fragments using infusion: a 3.6 kb U6-filler-gRNA scaffold-Ef1α sequence and a 2.9 kb vector backbone amplified from pBST-CRISPR using primers #6FR and #7FR, respectively, and a 0.5 kb gBlock containing a BseRI cloning site (#8). This intermediate was digested with BseRI and recombined with the 5.5 kb fragment of BE4-ΔBsmBI after EagI-PmeI treatment, generating pBS-BE4. A 6.6 kb pBS backbone-U6-filler-gRNA scaffold-Ef1α, sequence was PCR amplified from pBS-BE4 using #9FR, followed by infusion with NotI-AgeI-digested pCMV-ABEmax and pCMV_AncBE4max-ΔBsmBI, generating pBS-AncBE4max and pBS-ABEmax, respectively. Next, sgRNA oligos were synthesized, annealed and inserted into the BsmBI site of pBS-BE4, pBS-AncBE4max and pBS-ABEmax, generating shutter plasmids with all-in-one base editing components, such as pBS-ABEmax-sgHBG #2. Step 2) A 21.0 kb pHCAS3-MCS vector with PacI cloning site was generated similarly as described previously (Li et al., Cancer Res 80: 549-560, 2020) except that the stuffer DNA was trimmed down by EcoRI restriction and re-ligation with a 1.8 kb EcoRI fragment. A 2.2 kb PGK-MGMT^{P140K}-2A-GFP-bGHpolyA sequence was amplified from pHCA-Dual-MGMT-GFP (Li et al., Blood 131: 2915-2928, 2018) by #10FR and recombined with PacI-digested pHM5-FRT-IR-Eflcc-GFP (Richter et al., Blood 128: 2206-2217, 2016), resulting in pHM5-FI-PGK-MGMT-GFP. Subsequently, the fragment between I-CeuI and PI-SceI sites was transferred from this construct to the PshAI site of pHCAS3-MCS by #11FR and infusion cloning, forming pHCAS3-FI-PGK-MGMT-GFP-MCS. Step 3) The shuttle plasmids from step 1 and the resultant vector from step 2 was treated with PacI and recombined to generate the final constructs, such as pHCA-ABEmax-sgHBG #2-FI-MGMT-GFP. Final pHCA constructs with different sgRNA sequences were generated similarly except that different sgRNA were used in step 1.

Second version of ABE constructs: The second version of ABE constructs differs from the first version in promoters, alternative codon usage and miRNA-regulated gene expression. The cloning also involved 3 steps. Step 1) A 1.5 kb 3′ β-globin UTR with miR183/218 target sequence was amplified from pBST-sgHBG1-miR (Li et al, Blood 131: 2915-2928, 2018) using primers #12FR, followed by insertion into NotI-HpaI sites of pBS-ABEmax-sgHBG #2, generating pBS-ABEmax-sgHBG #2-miR. Shuttle plasmids for the second version of ABE constructs, for example, pBS-ABEopti-sgHBG #2-miR, were obtained by joining the following 4 fragments with Ascl-EcoRV-digested pBS-ABEmax-sgHBG #2-miR by infusion cloning: a human PGK promoter amplified from pHM5-FI-PGK-MGMT-GFP using #13FR, two gBlocks (#14 and #15) containing the two TadA genes with alternative codon usage to reduce sequence repetitiveness, and a 1.9 kb sequence amplified from pBS-ABEmax-sgHBG #2 using #16FR. Step 2) The SV40 polyA sequence between PshAI-NotI sites of pHM-FRT-IR-Ef1α-MGMT(P140K)-2A-GFP-pA was replaced with a bGH polyA sequence (gBlock #17), getting pHM-FI-Ef1α-MGMT(P140K)-GFP-bGHpA. Then, the whole 4.9 kb transposon between I-CeuI and PI-SceI sites was transferred to the PshAI site of pHCAS3-MCS using #11FR, generating pHCAS3-FI-Ef1α-MGMT-GFP-MCS. Step 3) The resultant constructs from step 1 and 2 were combined by infusion cloning following Pact treatment, generating pHCA-ABEopti-sgHBG #2-FI-MGMT-GFP. Final pHCA constructs with different sgRNA sequences were generated similarly.

The Phusion Hot Start II High-Fidelity DNA Polymerase was used in all PCR amplifications involved in cloning. Final constructs were screened by several restriction enzymes (HindIII, EcoRI and PmeI) and confirmed by sequencing the whole region containing transgenes.

For the production of HDAd5/35++ vectors, corresponding plasmids were linearized with PmeI and rescued in 116 cells (Palmer & Ng, Mol Ther 8: 846-852, 2003) with AdNG163-5/35++, an Ad5/35++ helper vector containing chimeric fibers composed of the Ad5 fiber tail, the Ad35 fiber shaft, and the affinity-enhanced Ad35++fiber knob (Richter et al, Blood 128: 2206-2217, 2016). HD-Ad5/35++ vectors were amplified in 116 cells as described in detail elsewhere (Palmer & Ng, Mol Ther 8: 846-852, 2003). Helper virus contamination levels were found to be <0.05%. Titers were 2-5×10¹² viral particles (vp)/mL.

Transfection of cell lines: 293FT (Thermo Fisher Scientific) and K562 cells were cultured according to the vendors' instructions. 293FT cells pre-seeded in 6-well plate were transfected with 4 μg plasmids (3 μg base editor or CRISPR/Cas9+1 μg pSP-sgBCL11AE (Li et al., Mol Ther Methods Clin Dev 9: 390-401, 2018)) using lipofectamine 3000 (Thermo Fisher Scientific) per the manufacturer's protocol. K562 cells were transfected with 2.66 μg plasmids (2 μg base editor or CRISPR/Cas9+0.6 μg pSP-sgBCL11AE) using nucleofection (Catalog #V4XC-2024) (Lonza, Basel, Switzerland) according to the provider's protocol. Genomic DNA was isolated at 4 days after transfection for analyses.

HUDEP-2 cells and erythroid differentiation: HUDEP-2 cells (Kurita et al., PloS One 8: e59890, 2013) were cultured in StemSpan SFEM medium (STEMCELL Technologies) supplemented with 100 ng/mL SCF, 3 IU/mL EPO, 10⁻⁶ M dexamethasone and 1 μg/mL doxycycline (DOX). Erythroid differentiation was induced in IMDM containing 5% human AB serum, 100 ng/mL SCF, 3 IU/mL EPO, 10 μg/mL Insulin, 330 μg/mL transferrin, 2 U/mL Heparin and 1 μg/mL DOX for 6 days.

Colony-forming unit (CFU) assay: The lineage minus (Lin⁻) cells were isolated by depletion of lineage-committed cells in bone marrow MNCs using the mouse lineage cell depletion kit (Miltenyi Biotec, San Diego, Calif.) according to the manufacturer's instructions. CFU assays were performed using ColonyGEL (Reachbio, Seattle, Wash.) with mouse complete medium according to the manufacturer's protocol. Colonies were scored 10 days after plating.

T7EI mismatch nuclease assay: Genomic DNA was isolated using PureLink Genomic DNA Mini Kit per provided protocol (Life Technologies, Carlsbad, Calif.) (Miller et al., Nat Biotechnol 25: 778-785, 2007). A genomic segment encompassing the target site of erythroid BCL11A enhancer was amplified by PCR primers: BCL11A forward (SEQ ID NO: 247) and reverse (SEQ ID NO: 263). PCR products were hybridized and treated with 2.5 Units of T7E1 (New England Biolabs) for 30 minutes at 37° C. Digested PCR products were resolved by 10% TBE PAGE (Bio-Rad) and stained with ethidium bromide. 100 bp DNA Ladder (New England Biolabs) was used. Band intensity was analyzed using ImageJ software. % cleavage=(1-sqrt(parental band/(parental band+cleaved bands))×100%.

Flow cytometry: Cells were resuspended at 1×10⁶ cells/100 μL in FACS buffer (PBS, 1% FBS) and incubated with FcR blocking reagent (Miltenyi Biotech, Auburn Calif.) for ten minutes on ice. Next the staining antibody solution was added in 100 μL per 10⁶ cells and incubated on ice for 30 minutes in the dark. After incubation, cells were washed once in FACS buffer. For secondary staining the staining step was repeated with a secondary staining solution. After the wash, cells were resuspended in FACS buffer and analyzed using a LSRII flow cytometer (BD Biosciences, San Jose, Calif.). Debris was excluded using a forward scatter-area and sideward scatter-area gate. Single cells were then gated using a forward scatter-height and forward scatter-width gate. Flow cytometry data were then analyzed using FlowJo (version 10.0.8, FlowJo, LLC). For analysis of LSK cells, cells were stained with biotin-conjugated lineage detection cocktail (catalog #130-092-613) (Miltenyi Biotec, San Diego, Calif.), antibodies against c-Kit (clone 2B8, catalog #12-1171-83) and Sca-1 (clone D7, catalog #25-5981-82), followed by secondary staining with APC-conjugated streptavidin (catalog #17-4317-82) (eBioscience, San Diego, Calif.). Other antibodies from eBioscience included anti-mouse CD3-APC (clone 17A2) (catalog #17-0032-82), anti-mouse CD19-PE-Cyanine7 (clone eBio1D3) (catalog #25-0193-82), and anti-mouse Ly-66 (Gr-1)-PE, (clone RB6-8C5) (catalog #12-5931-82. Anti-mouse Ter-119-APC (clone Ter-119) (catalog #116211) was from Biolegend (San Diego, Calif.).

Intracellular flow cytometry detecting human γ-globin expression: The FIX & PERM™ cell permeabilization kit (Thermo Fisher Scientific) was used and the manufacture's protocol was followed. Briefly, 5×10⁶ HUDEP-2 cells were resuspended in 100 μL FACS buffer. 100 μL of reagent A (fixation medium) was added and incubated for 2-3 minutes at room temperature. 1 mL pre-cooled absolute methanol was then added, mixed and incubated on ice in the dark for 10 minutes. The samples were then washed with FACS buffer, resuspended in 100 μL reagent B (permeabilization medium) with 0.6 μg hemoglobin γ antibody (Clone 51-7, catalog #sc-21756 PE) (Santa Cruz Biotechnology, Dallas, Tex.), and incubated for 30 minutes at room temperature. After wash, cells were resuspended in FACS buffer and analyzed.

Globin HPLC: Individual globin chain levels were quantified on a Shimadzu Prominence instrument with a SPD-10AV diode array detector and an LC-10AT binary pump (Shimadzu, Kyoto, Japan). Vydac 214TP™ C4 Reversed-Phase columns for polypeptides (214TP54 Column, C4, 300 A, 5 μm, 4.6 mm i.d.×250 mm) (Hichrom, UK) were used. A 40%-60% gradient mixture of 0.1% trifluoroacetic acid in water/acetonitrile was applied at a rate of 1 mL/min.

Measurement of vector copy number: For absolute quantification of adenoviral genome copies per cell, genomic DNA was isolated from cells using PureLink Genomic DNA Mini Kit per provided protocol (Life Technologies), and used as template for qPCR performed using the power SYBR™ green PCR master mix (Thermo Fisher Scientific). The following primer pairs were used: MGMT forward (SEQ ID NO: 220), and reverse (SEQ ID NO: 221).

Real-time reverse transcription PCR: Total RNA was extracted from 5×10⁶ differentiated HUDEP-2 cells or 100 μL blood by using TRIzol™ reagent (Thermo Fisher Scientific) followed by phenol-chloroform extraction. QuantiTect reverse transcription kit (Qiagen) and power SYBR™ green PCR master mix (Thermo Fisher Scientific) were used. Real time quantitative PCR was performed on a StepOnePlus real-time PCR system (AB Applied Biosystems). The following primer pairs were used: mouse RPL10 (house-keeping) forward (SEQ ID NO: 189), and reverse (SEQ ID NO: 190); human γ-globin forward (SEQ ID NO: 191), and reverse (SEQ ID NO: 192); human β-globin forward (SEQ ID NO: 216), and reverse (SEQ ID NO: 217); mouse β-major globin forward (SEQ ID NO: 193), and reverse (SEQ ID NO: 194), mouse a globin forward (SEQ ID NO: 212), and reverse (SEQ ID NO: 213).

Detection of base editing: Genomic DNA was isolated as described above. Genomic segments encompassing the target sites of BCL11A enhancer and HBG1/2 promoter were amplified with KOD Hot Start DNA Polymerase (MilliporeSigma) using primers: HBG1 forward (SEQ ID NO: 31), reverse (SEQ ID NO: 33); HBG2 forward (SEQ ID NO: 69), reverse (SEQ ID NO: 72); and BCL11A primers shown above. The amplicons were purified by using the NucleoSpin Gel & PCR Clean-up kit (Takara) and sequenced with the following primers: HBG1-seq (SEQ ID NO: 105); HBG2-seq (SEQ ID NO: 237); and BCL11A-seq (SEQ ID NO: 247). The base editing level was quantified from Sanger sequencing results by using EditR 1.0.9 (Kluesner et al., CRISPR J 1: 239-250, 2018).

Animal studies: All experiments involving animals were conducted in accordance with the institutional guidelines set forth by the University of Washington. The University of Washington is an Association for the Assessment and Accreditation of Laboratory Animal Care International (AALAC)-accredited research institution and all live animal work conducted at this university is in accordance with the Office of Laboratory Animal Welfare (OLAVV) Public Health Assurance (PHS) policy, USDA Animal Welfare Act and Regulations, the Guide for the Care and Use of Laboratory Animals and the University of Washington's Institutional Animal Care and Use Committee (IACUC) policies. The studies were approved by the University of Washington IACUC (Protocol No. 3108-01). C57BL/6J based transgenic mice that contain the human CD46 genomic locus and provide CD46 expression at a level and in a pattern similar to humans (hCD46^{+/+} mice) were described earlier (Kemper et al., Clin Exp Immunol 124: 180-189, 2001). Transgenic mice carrying the wildtype 248 kb β-globin locus yeast artificial chromosome (β-YAC) were used (Peterson et al., Ann NY Acad Sci 850: 28-37, 1998). 13-YAC mice were crossed with human CD46^{+/+} mice to obtain p-YAC^{+/-}/CD46^{+/+} mice for in vivo HSPC transduction studies. The following primers were used for genotyping of mice: CD46 forward (SEQ ID NO: 233) and reverse (SEQ ID NO: 234); β-YAC (γ-globin promoter) forward (SEQ ID NO: 242) and reverse (SEQ ID NO: 243).

HSPC mobilization and in vivo transduction: HSPCs were mobilized in mice by subcutaneous (SC) injections of human recombinant G-CSF (5 μg/mouse/day, 4 days) followed by an SC injection of AMD3100 (5 mg/kg) on day 5. In addition, animals received Dexamethasone (10 mg/kg, IP) 16 h and 2 h before virus injection. Thirty and 60 minutes after AMD3100, animals were intravenously injected with virus vectors through the retro-orbital plexus with two doses of viruses (4×10¹⁰ vp/dose×2 doses). The base editing and SB viruses were co-delivered at a 1:1 ratio.

In vivo selection: Selection was started at one week (Townes model) or four weeks (β-YAC model) after transduction. Mice were injected with O⁶-BG (15 mg/kg, IP) two times, 30 minutes apart. One hour after the second injection of O⁶-BG, mice were injected (IP) with 5 mg/kg BCNU. At two and four weeks after the first round of selection, two more rounds were performed with BCNU doses at 7.5 and 10 mg/kg, respectively.

Secondary bone marrow transplantation: Recipients were female C57BL/6J mice, 6-8 weeks old from the Jackson Laboratory. On the day of transplantation, recipient mice were irradiated with 1000 Rad. Bone marrow cells from in vivo transduced CD46tg mice were isolated aseptically and lineage-depleted cells were isolated using MACS as described above. Six hours after irradiation cells were injected intravenously at 1×10⁶ cells per mouse. The secondary recipients were kept for 16 weeks after transplantation for terminal point analyses.

Tissue analyses: Spleen and liver tissue sections of 2.5 μm thickness were fixed in 4% formaldehyde for at least 24 hours, dehydrated and embedded in paraffin. Staining with hematoxylin-eosin was used for histological evaluation of extramedullary hemopoiesis. Hemosiderin was detected in tissue sections by Perl's Prussian blue staining. Briefly, the tissue sections were treated with a mixture of equal volumes (2%) of potassium ferrocyanide and hydrochloric acid in distilled water and then counterstained with neutral red.

Blood analyses: Blood samples were collected into EDTA-coated tubes and analysis was performed on a HemaVet 950FS (Drew Scientific, Waterbury, Conn.). Peripheral blood smears were stained with Giemsa/May-Grunwald (Merck, Darmstadt, Germany) for 5 and 15 minutes, respectively. Reticulocytes were stained with Brilliant cresyl blue. The investigators who counted the reticulocytes on blood smears have been blinded to the sample group allocation. Only animal numbers appeared on the slides (five slides per animal, five random 1 cm² sections).

Statistical analyses: For comparisons of multiple groups, one-way and two-way analysis of variance (ANOVA) with Bonferroni post-testing for multiple comparisons were employed. Statistical analysis was performed using Graph Pad Prism version 6.01 (GraphPad Software Inc., La Jolla, Calif.).

Results. Selection of base editors and guide RNAs. The editing activity of multiple versions of cytidine base editors (CBE) were compared including BE4 (Komo et al., Science Advances 3: eaao4774, 2017), AncBE4max (Koblan et al., Nature Biotechnology 36: 843-846, 2018), BE3RA and FNLS (Zafra et al., Nature Biotechnology 36: 888-893, 2018). The base editors (BEs) were subcloned and driven by an ubiquitous EF1α promoter. A second plasmid expressing guide RNA under a human U6 promoter that targets the GATAA motif in the +58 BCL11A enhancer region (Canver et al., Nature 527: 192-197, 2015) was used for co-transfection. Although the BE3RA showed higher editing in 293FT cells (FIG. 131A), the AncBE4max system demonstrated the highest activity in K562 erythroid cells measured by cleavage assay (FIG. 131B). Therefore, AncBE4max was used in downstream studies. For adenine base editor (ABE), the ABEmax system developed by David Liu group was used and optimized using a similar approach as for AncBE4max (Koblan et al., Nature Biotechnology 36: 843-846, 2018). The xCas9(3.7)-BE4 and xCas9(3.7)-ABE(7.10) editors were also used in guide sequence screening due to their broad PAM compatibility (Hu et al., Nature 556: 57-63, 2018).

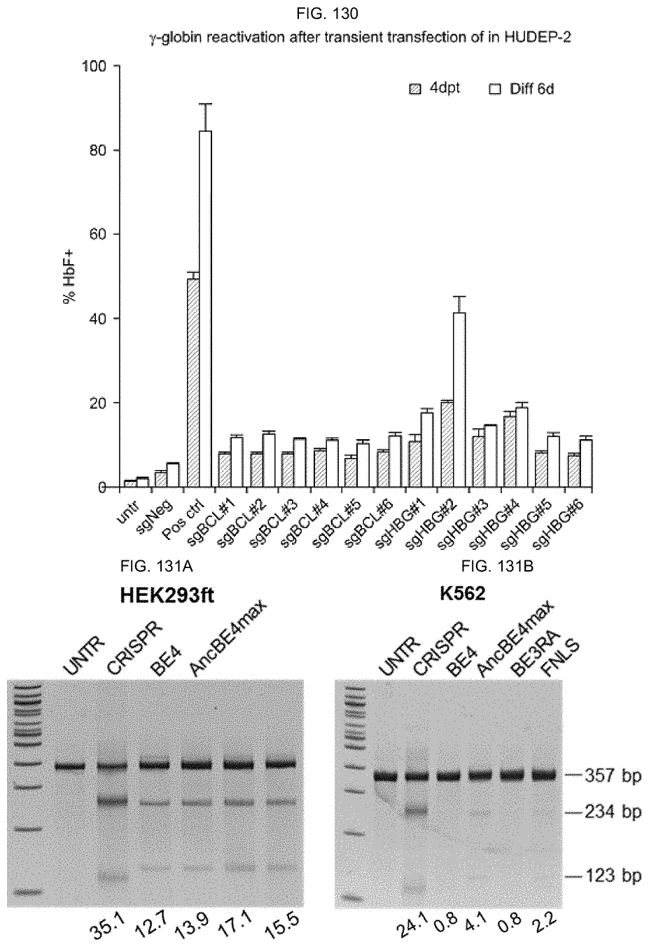

The optimal targetable window of base editors is position 4-8 of the protospacer, counting the 5′ end first base as position 1. A panel of single guide RNA (sgRNA) sequences were designed specific to the GATAA motif in the +58 BCL11A enhancer (sgBCL #1 to #6) or recreating various naturally occurring Hereditary Persistence of Fetal Hemoglobin (HPFH) mutations in the HBG1/2 promoter (sgHBG #1 to #6). The sequences and their specific target motifs/bases were shown in Table 14. The guide sequences were tested in an erythroid progenitor cell line HUDEP-2 cells (Kurita et al., PloS One 8: e59890, 2013) for their potency to reactivate γ-globin expression. The cells were put into erythroid differentiation at day 4 after transfection. All 12 sgRNA sequences led to significant γ-globin expression compared to a negative CBE control that targets CCR5 expression but not hemoglobin-related genes (FIG. 130). sgHBG #2 resulted in 41% HbF⁺ cells at day 6 after differentiation. A previously described CRISPR vector targeting the BCL11A binding site in HBG promoter was used as a positive control and generated 84% of HbF⁺ cells (Li et al., Blood 131: 2915-2928, 2018). Accordingly, sgBCL #1 (CBE), sgHBG #1 (CBE), sgHBG #2 (ABE) and sgHBG #4 (ABE) were chosen for viral vector delivery in consideration of their activity as well as diversity of target sites. The negative control vector sgNeg (CBE) and a vector containing both sgHBG #1 and sgBCL #1 (Dual, CBE) were also constructed.

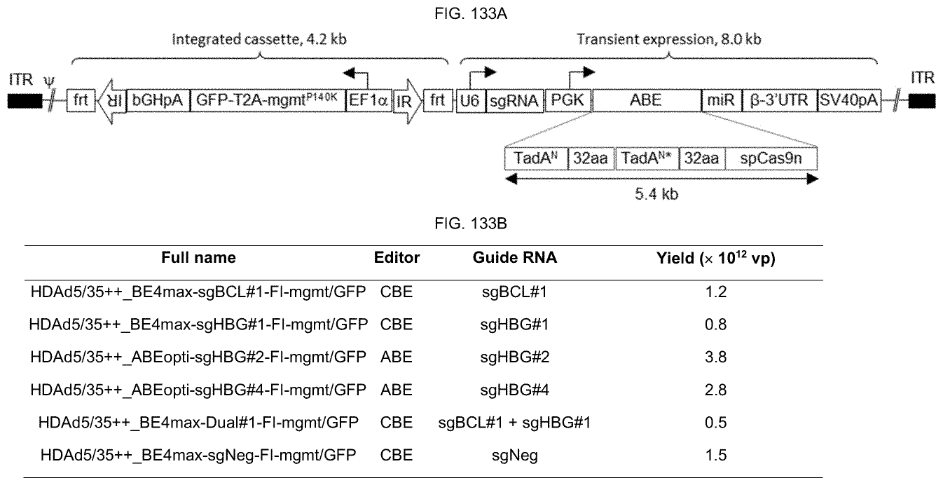

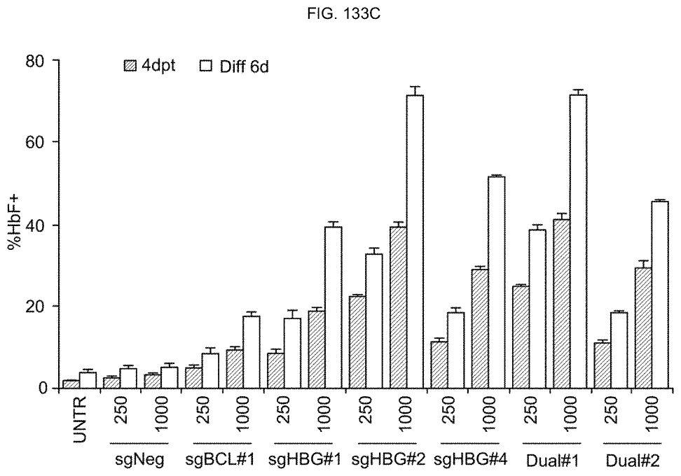

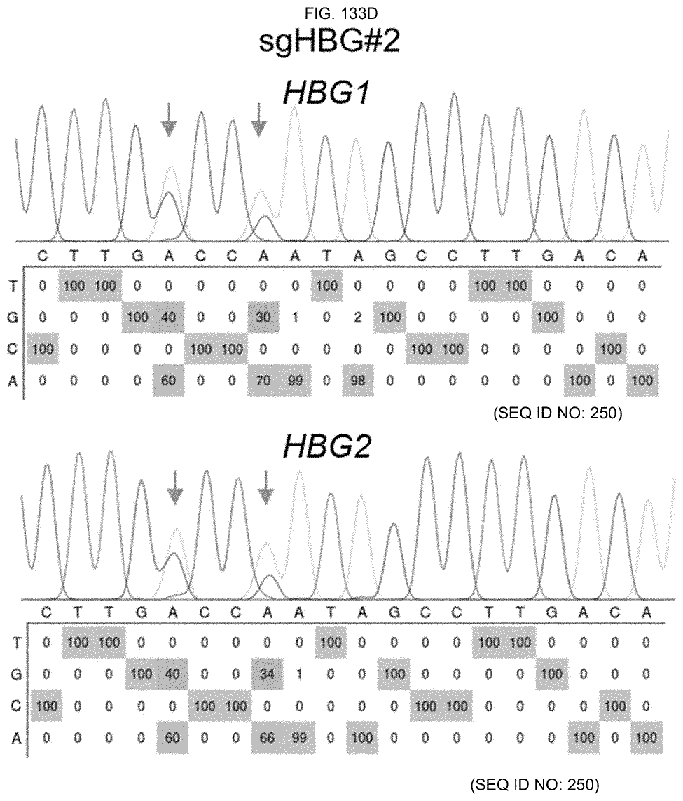

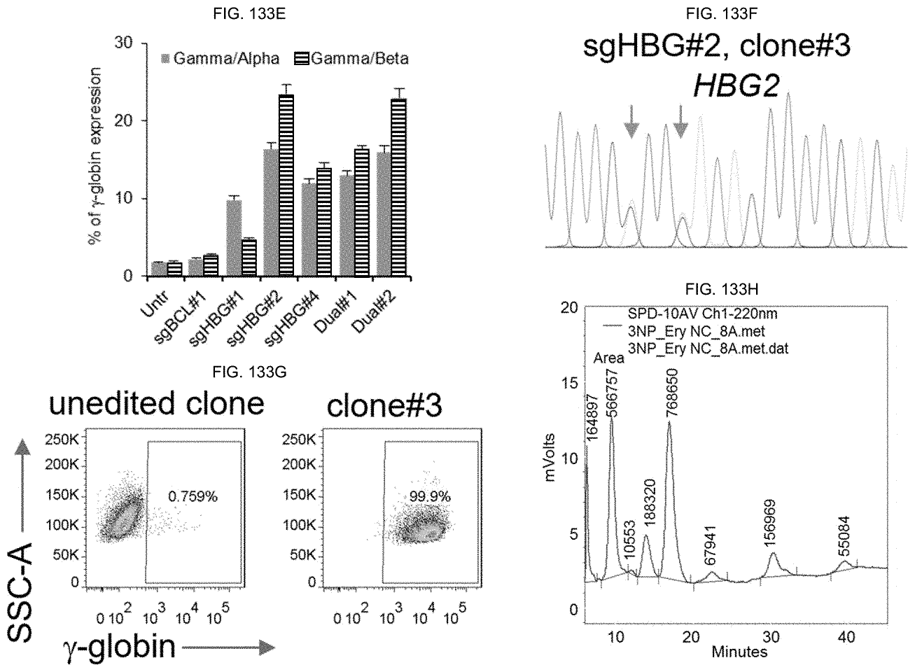

Generation of help-dependent adenovirus vectors (HDAd) expressing BE. Next, the aim was to produce viral vectors for efficient in vivo BE delivery. Due to the over 8 kb size of base editors with necessary regulatory elements, it is difficult to fit into one lentiviral vector (LV) or adeno-associated vector (AAV). HDAd vectors were developed with modified fiber, called HDAd5/35++, for efficient transduction of hematopoietic stem cells (HSCs) (Li et al., Mol Ther Methods Clin Dev 9: 142-152, 2018). HDAd vectors can accommodate 36 kb packaging capacity, providing ample space for BE components. In the first attempt, the BE enzyme (rAPOBEC1-nCas9-2xUGl for CBE or 2xTadA-nCas9 for ABE) was placed under an EF1a, promoter. The whole BE components including sgRNA driven by a human U6 promoter were cloned into the HDAd vector plasmid pHCA. A MGMT/GFP cassette flanked by FRT and transposon sites was also cloned to the vector to facilitate selection of transduced cells by O6BG/BCNU treatment (FIGS. 132A and 132B). Notably, the BE components were placed outside of the transposon. This design allowed for i) transient expression of the BE while maintaining integrated expression of MGMT/GFP; and ii) more rapid degradation of editing enzymes upon co-infection with another vector expressing sleepy beauty transposase (HDAd-SB) (for further discussion and/or additional illustration of certain aspects of vector design, see also Example 3). Although the yield per 3-liter spinner was relatively low (1×10¹² viral particles or vp on average), all four CBE vectors were rescued. This is in contrast to HDAd-CRISPR vectors that are not rescuable without mechanisms to regulate nuclease expression (Saydaminova et al., Mol Ther Methods Clin Dev 1: 14057, 2015). The results suggested that DSB-free BE system may be less toxic to the HDAd producer cells than CRISPR/Cas9. For the ABE vectors, the viruses appeared rearranged and no distinct HDAd band was observed after ultracentrifugation with CsCl gradient. Since the major difference between ABE and CBE vectors are the deaminase region, it was likely that the two TadA-32aa repeats in ABE vectors were the causative elements. Therefore, the following modifications were made to the first version of ABE vectors: i) the sequence repetitiveness between the two TadA-32aa repeats was further reduced by alternative codon usage (FIG. 132C); ii) A PGK promoter was used to drive the BE enzyme. While being constitutive in HSCs (Li et al., Cancer Res 80: 549-560, 2020), the PGK promoter drives lower gene expression than Ef1α in 116 producer cells (Qin et al., PloS One 5: e10611, 2010), eliminating potential TadA-associated adverse effects; iii) A miR183/218-based gene regulation system was utilized to further control BE expression (Saydaminova et al., Mol Ther Methods Clin Dev 1: 14057, 2015) (FIG. 133A). This second version of constructs with optimized design led to successful rescue of two HDAd-ABE viruses with an average yield of 3.3×10¹² vp/spinner which are within the normal yield range (FIG. 133B).

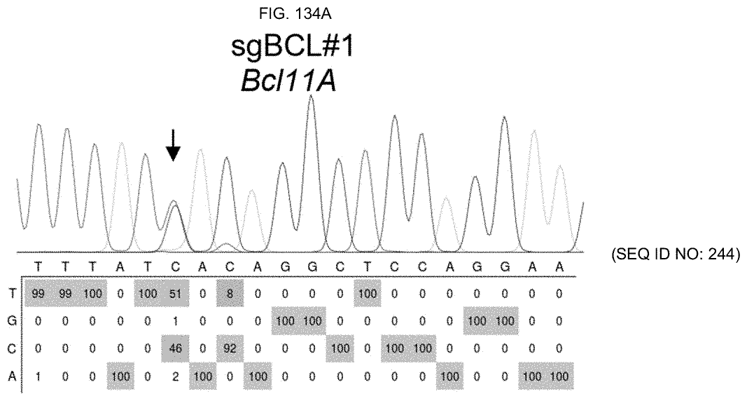

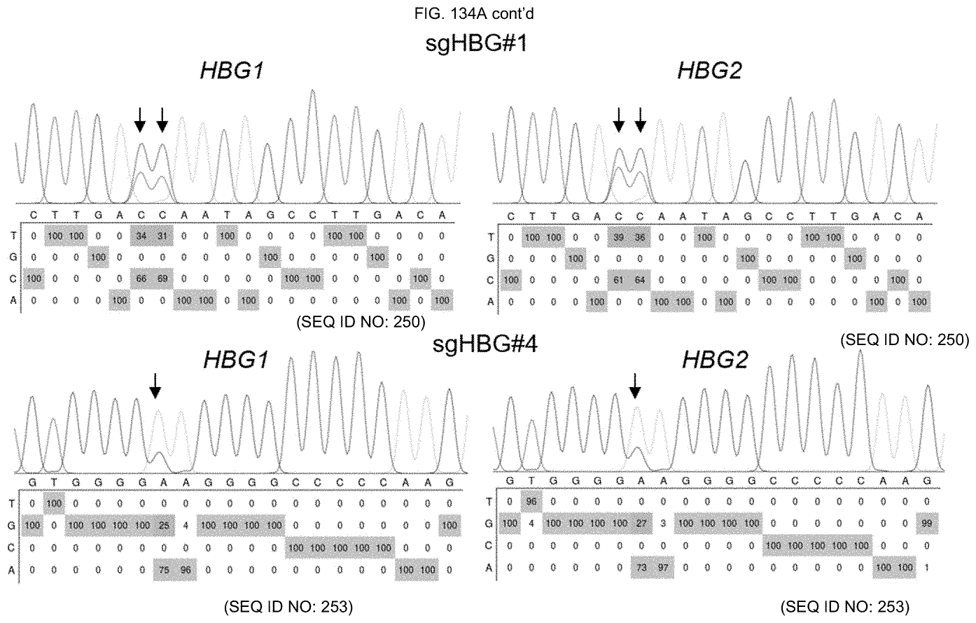

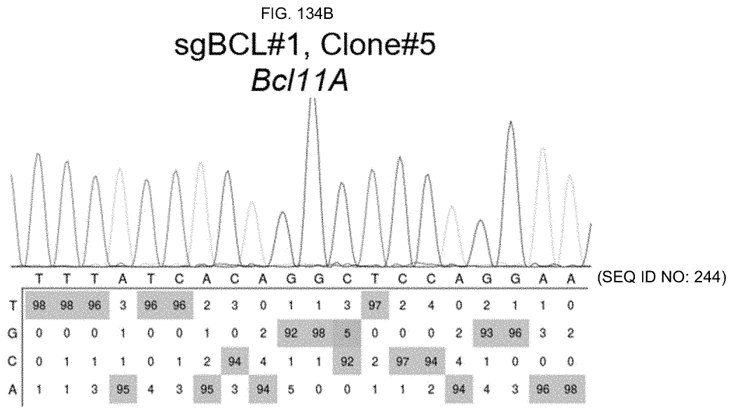

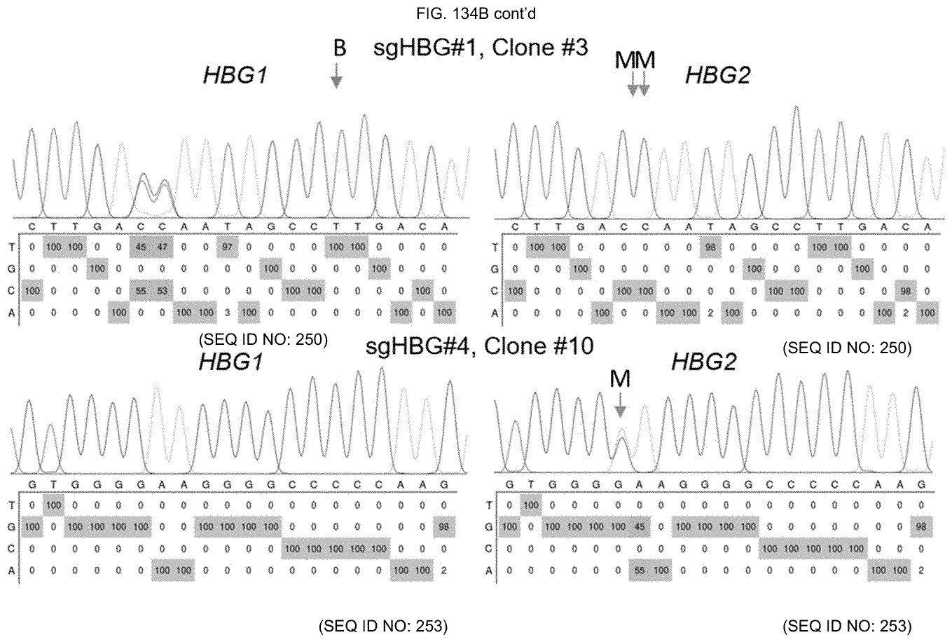

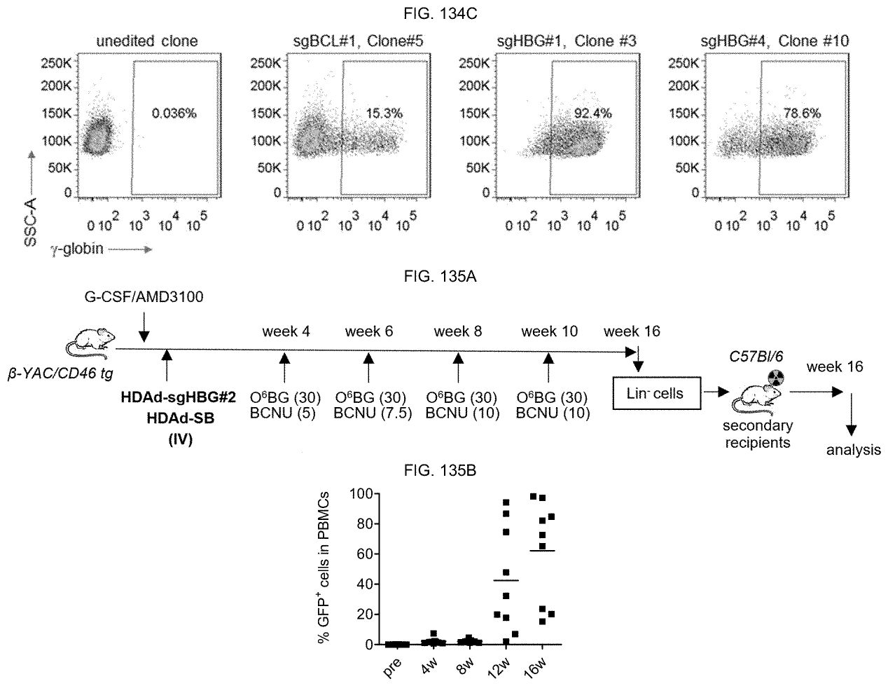

The HDAd vectors were examined next in HUDEP-2 cells. All five tested vectors efficiently installed target base conversion and led to substantial γ-globin reactivation (FIG. 133 and FIG. 134). Consistent with screening data by transient transfection, HDAd-ABE-sgHBG #2 vector induced the highest level of HbF⁺ cells (71% at MOI 1000 vp/cell). Interestingly, while sgBCL #1 and sgHBG #1 alone mediated 17% and 39% HbF⁺ cells, respectively, the Dual targeting vector simultaneously expressing sgBCL #1 and sgHBG #1 generated HbF induction at a level comparable to that of sgHBG #2 (FIG. 133C), indicating a synergistic effect. No significant HbF induction was measured for the negative control vector. γ-globin protein levels measured by HPLC were consistent with flow cytometry data. 23% of human γ-globin over human β-globin was observed following transduction with sgHBG #2, demonstrating significant switching (FIGS. 133E and 133H). At MOI 1000, base conversion frequencies for the four sgRNAs were in the range of 25-51% (FIG. 133D and FIG. 134A). For sgHBG #2, 40% and 34% A >G conversion at position 5 and 8 was detected, respectively (FIG. 133D). The As to G conversion simulated -113A >G HPFH mutation (Table 14) (Martyn et al., Blood 133(8):852-856, 2019). No significant editing difference was found between HBG1 and HBG2. In single-cell derived clones, monoallelic edits at A₅ and A₈ sites conferred 100% of HbF-positive cells (FIGS. 133F and 133G), confirming the critical role of these sites in regulating HbF suppression. Similar results were shown in clones derived from sgHBG #1 and sgHBG #4. In clones transduced with sgBCL #1, a biallelic G >A mutation in the GATAA motif of BCL11A enhancer led to 15% HbF-expressing cells (FIGS. 134B and 134C). Collectively, these data demonstrate that HDAd-BE vectors specific to critical sites in the BCL11A enhancer or HBG1/2 promoter can efficiently reactivate HbF expression.

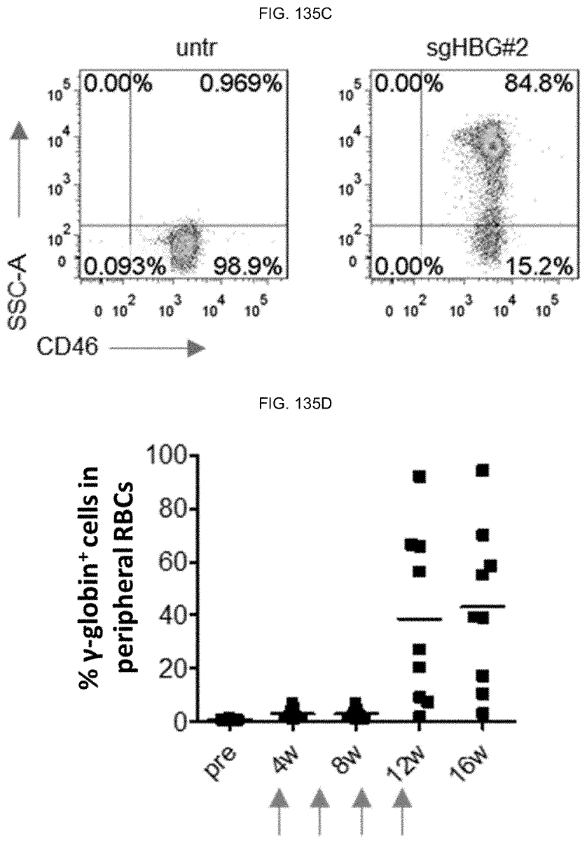

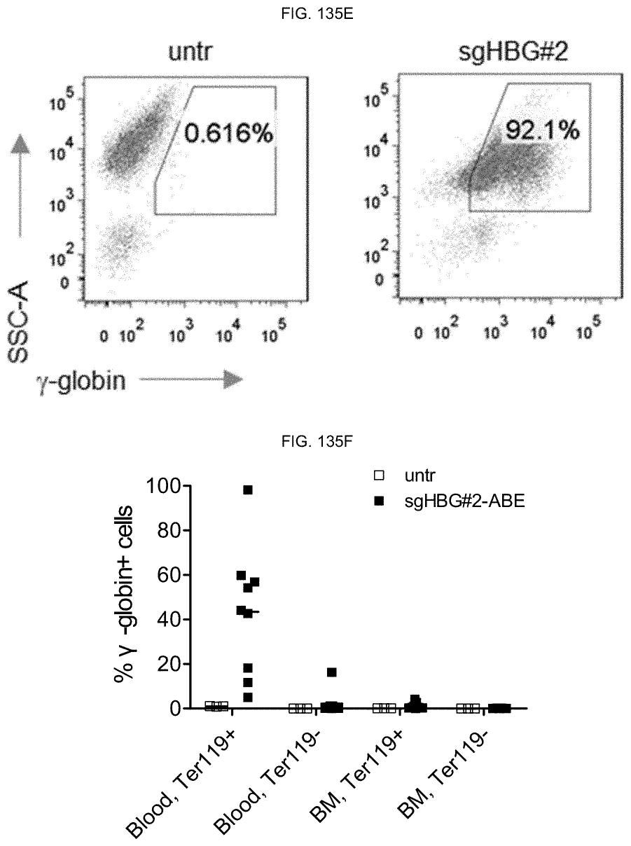

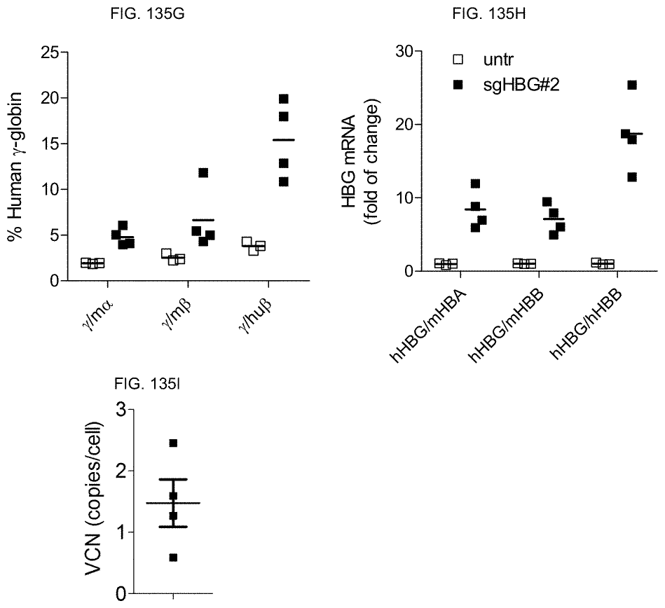

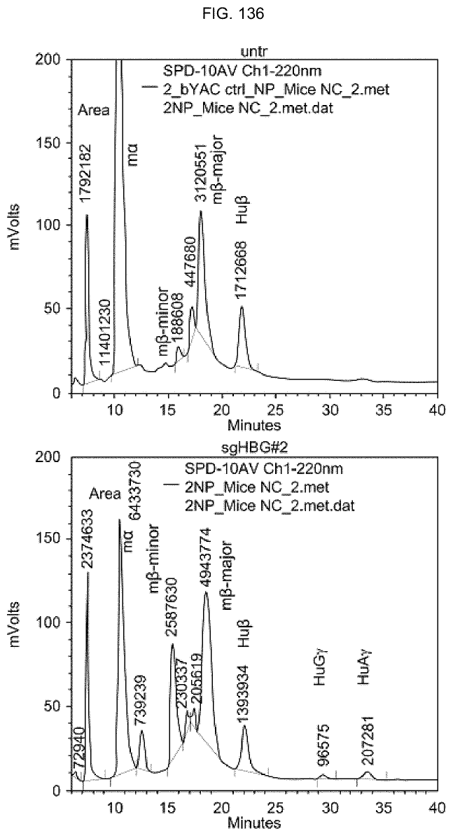

Reactivation of γ-globin in β-YAC mice following in vivo transduction with base editors. A simplified gene therapy approach was established by in vivo transduction of HSCs with HDAd5/35++vectors (Richter et al., Blood 128: 2206-2217, 2016). Therefore, the efficacy of base editing with this novel in vivo strategy was investigated. β-YAC mice were used that contain a 248 kb of human DNA including the complete 82 kb β-globin locus (Peterson et al., PNAS USA 90: 7593-7597, 1993). The mice were crossed with human CD46 transgenic mice to allow for transduction with HDAd5/35++ vectors. The HDAd-ABE-sgHBG #2 was selected due to its highest efficacy to induce γ-globin expression in HUDEP-2 cells. Following mobilization with G-CSF/Plerixafor, β-YAC/CD46 mice were intravenously injected with HDAd-ABE-sgHBG #2 and HDAd-SB vectors. Four weeks after transduction, mice were subjected to four rounds of O⁶BG/BCNU (O⁶-Benzylguanine/Carmustine) treatment to selectively expand progenitors with integrated MGMT-GFP transgenes (FIG. 135A). After selection, the GFP marking in PBMCs reached 60% (FIGS. 135B and 135C). Notably, γ-globin expression in peripheral blood cells was raised from 1% before transduction to 43% on average (n=9) at week 16 after transduction, demonstrating significant γ-globin reactivation (FIGS. 135D and 135E). The large variation existed among different mice was probably caused by the bicistronic design of MGMT-2A-GFP that might result in lower expression of MGMT and therefore affected in vivo selection efficacy. γ-globin⁺ cells largely resided in the red blood cell (RBC) fraction (Ter-119+) in both blood at bone marrow samples (FIG. 135F). In RBC lysate at week 16, up to 21% of γ-globin over human β-globin protein was measured by high performance liquid chromatography (HPLC) (FIG. 135G and FIG. 136). γ-globin mRNA expression was in line with HPLC data (FIG. 135H). In total bone marrow mononuclear cells at week 16, the integrated vector copy number was up to 2.5 copies/cell (1.4 on average) (FIG. 135I).

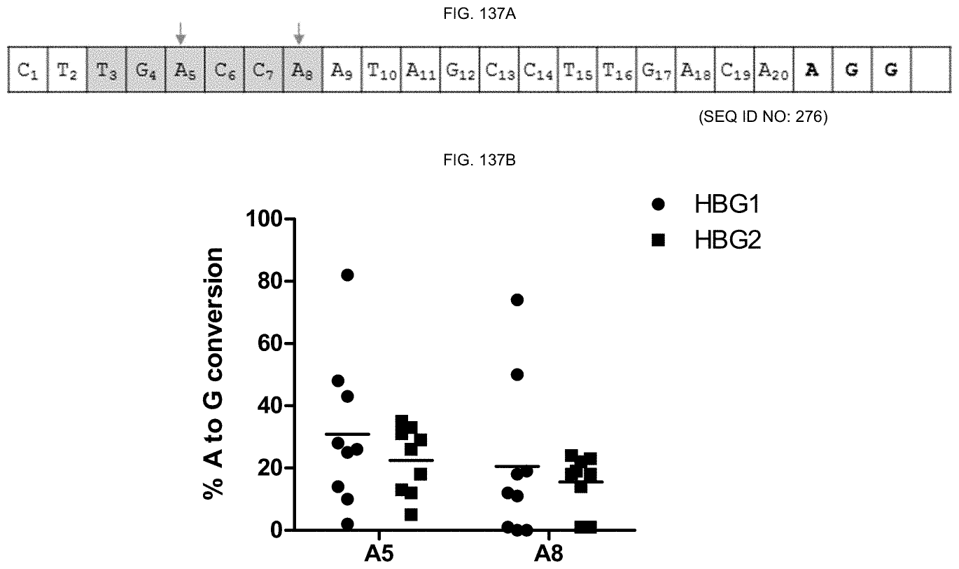

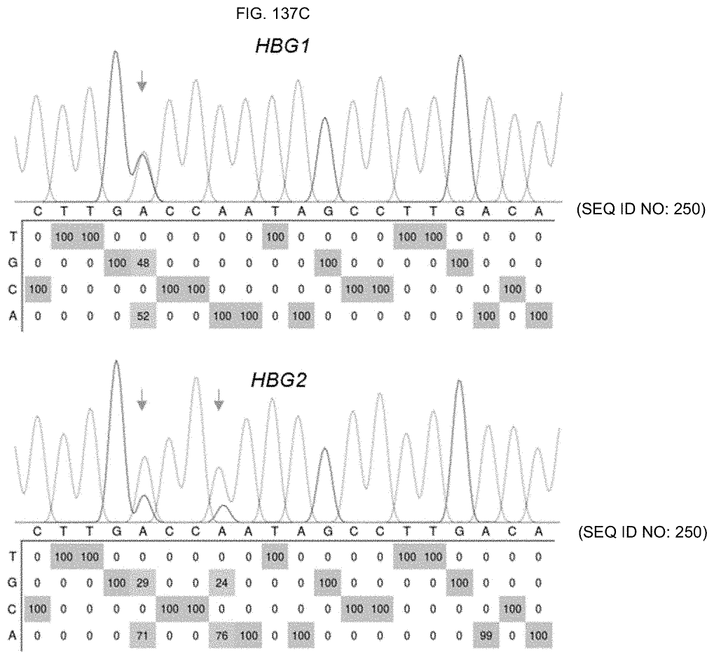

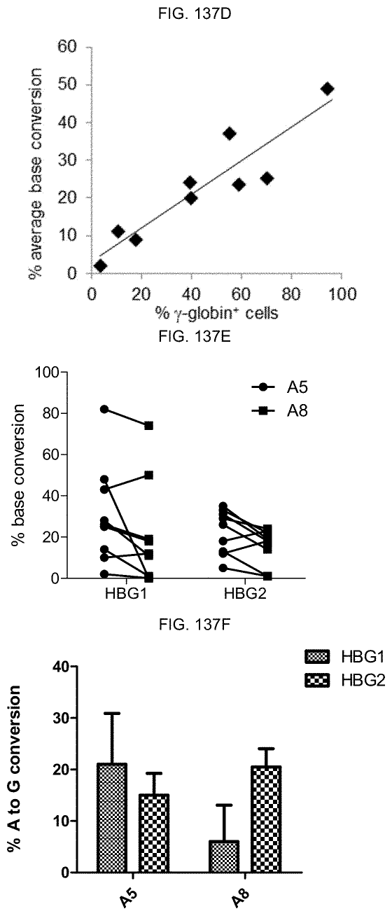

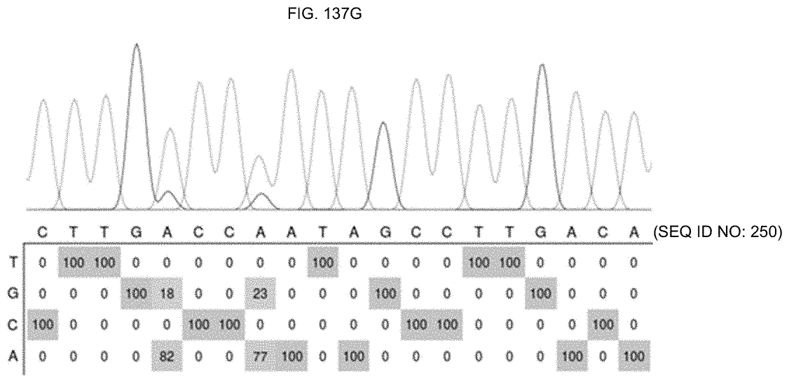

Base edits in the HBG1/2 promoter were analyzed. The A >G conversion frequencies at A₅ and A₈ sites in HBG1 and HBG2 were on average 15-30% (FIGS. 137A-137C). The base editing frequency was found to be tightly correlated with the level of γ-globin expression (Pearson test, R=0.92, p<0.001) (FIG. 137D). In the mouse with highest γ-globin expression, 82% target base conversion was achieved (FIG. 137B). Of note, there was a tendency that the conversion % at A₅ was slightly higher than that at A₈ site in both HBG1 and HBG2 regions, though no statistical difference was found (FIG. 137B). It has been showed that some base editors exhibit processive editing when multiple targets present in the protospacer. However, no editing was found at the A₉ site (FIGS. 137A and 137C). This was likely because position 9 is located outside of the optimal editing window, demonstrating the narrowness of editable window.

In summary, these data demonstrate that in vivo transduction with base editors specific to the HBG1/2 promoter followed by selection leads to efficient target base conversion and γ-globin reactivation in β-YAC/CD46 mice.

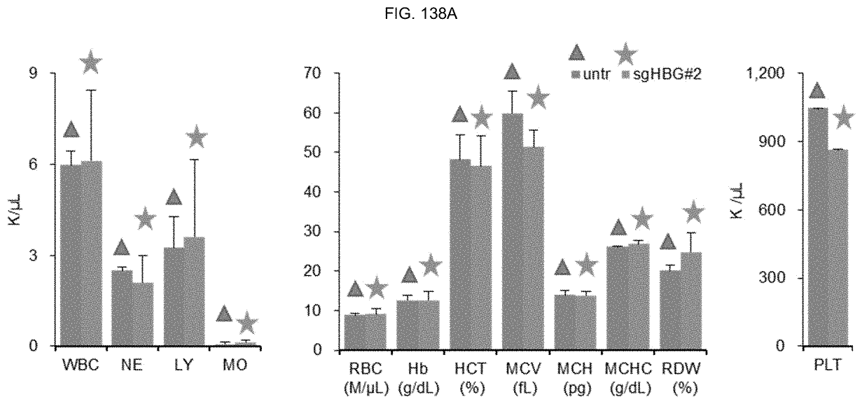

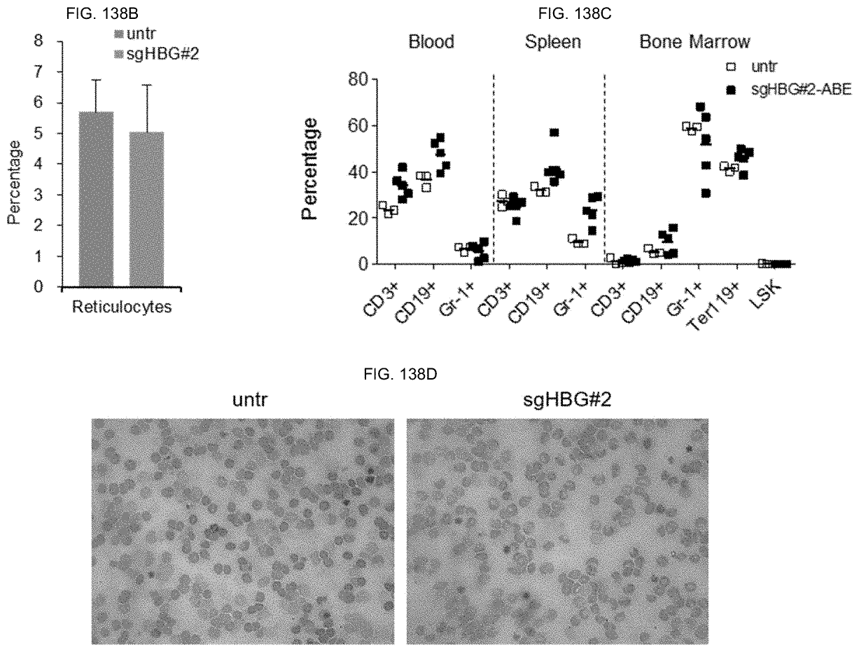

Good safety profile and stable efficacy after in vivo HSC base editing. At week 16, the animals were euthanized and tissue samples were subjected to multiple hematology and histology analyses. Hematological parameters, including white blood cells (K/μL), red blood cells (M/μL), Hb (g/dL), MCV (fL), MCHC (g/dL), RDW (%) and platelets (K/μL), were similar to that of naïve β-YAC/CD46 mice (FIGS. 138A and 138B). The percentage of reticulocytes in peripheral blood measured by Brilliant cresyl blue staining was comparable to mice without treatment (FIG. 138D). No foci of extramedullary erythropoiesis were observed on spleen and liver sections. The cellular composition in PBMCs, spleen and bone marrow mononuclear cells was revealed to be indistinguishable from control mice (FIG. 138C). Besides, compared to other previously reported gene therapy vectors (Li et al., Blood 131: 2915-2928, 2018; Wang et al., J Clin Invest. 129(2): 598-615, 2018; Li et al., Molecular Therapy 27: 2195-2212, 2019), HDAd-ABE-sgHBG #2 did not cause obvious change of body weight, behavior and appearance after in vivo transduction/selection.

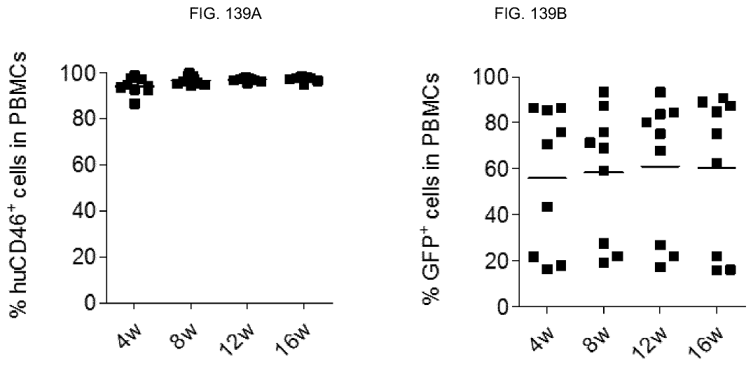

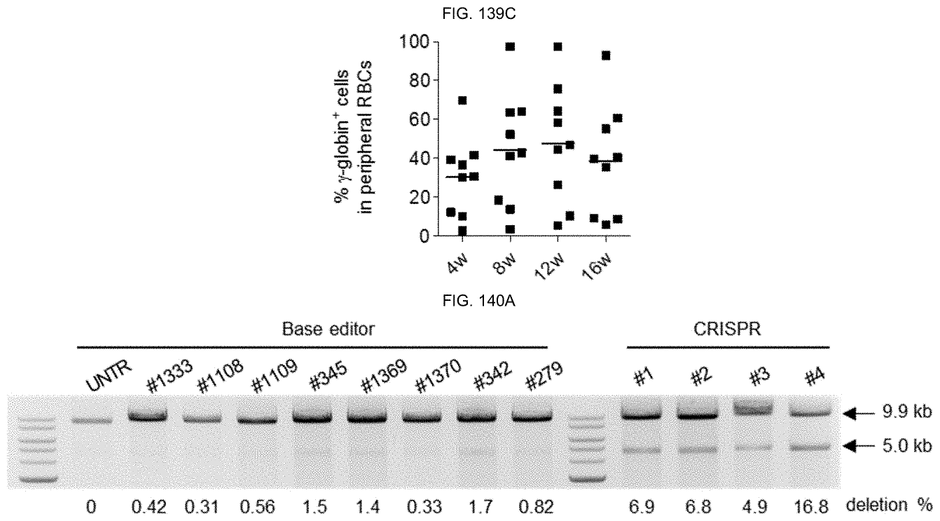

To demonstrate that in vivo transduction occurred in long-term repopulating HSPCs, bone marrow lineage minus (Lin⁻) cells harvested at week 16 were transplanted after transduction into lethally irradiated C57BL/6J mice (without the human CD46 gene). The ability of transplanted cells to drive the multi-lineage reconstitution in secondary recipients was evaluated over a period of 16 weeks. Engraftment rates based on huCD46 expression in PBMCs were over 95% and remained stable (FIG. 139A). GFP marking of PMBCs was comparable to that in primary mice (FIG. 139B). The percent of γ-globin⁺ RBCs was on average 40% and stable (FIG. 139C).

These observations together demonstrated that in vivo HSC base editing was overall safe. The modified HSPCs persisted long term and were capable of reconstituting secondary recipient mice with stable transgene expression.

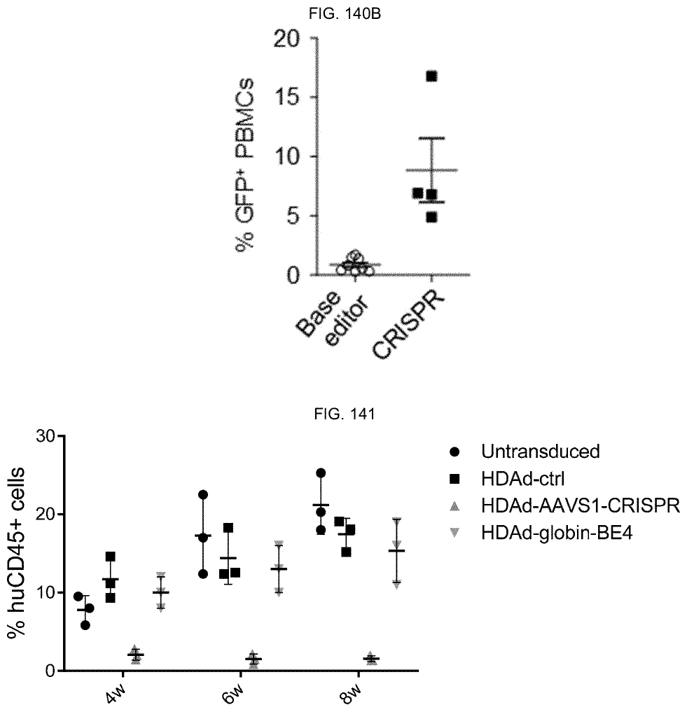

Minimal intergenic deletion and no detectable editing at top-scored off-target sites. A trade-off of DSB-dependent gene editing strategies is potential genomic large-fragment deletion (Kosicki et al., Nature Biotechnology 36: 765, 2018). In the case of targeting HBG1/2 promoters by DSB-generating nucleases, this side effect may become more significant due to the high similarity between the HBG1 and HBG2 regions. Guide sequences specific to one of the two regions may also target the other one. It has been reported that targeting the BCL11A binding sites in HBG1/2 promoters with CRISPR/Cas9 leads to a 4.9 kb intergenic deletion (Traxler et al., Nature Medicine 22: 987-990, 2016; Li et al., Blood 131: 2915-2928, 2018). As a result, the whole HBG2 gene is removed. Therefore, the genomic deletion by a semi-quantitative PCR was looked into (Li et al., Blood 131: 2915-2928, 2018). A pair of primers flanking the two targeting sites was used to amplify a 9.9 kb genomic segment. The presence of 4.9 kb deletion would generate an extra shortened 5.0 kb PCR amplicon. The percentage of deletion was positively correlated with the ratio of 5.0 kb to 9.9 kb amplicons by establishing a standard curve (see FIG. 7C in Li et al., Blood 131: 2915-2928, 2018). It was found that the average 4.9 kb deletion in base editor-treated mice was below 1% (FIG. 140). In some mice, it was barely detectable. This was significantly lower than that derived from transduction with an HDAd-HBG-CRISPR vector (Li et al., Blood 131: 2915-2928, 2018).

Next, off-target analyses was conducted to examine the fidelity of the system. In silico analysis showed no potential off-target sites with ≤5. 2 base pairs (bp) mismatches to the guide sequence in both human and mice genome. There were 10 and 2 potential off-targets with 3 bp mismatches in human and mice, respectively. It was speculated that the likelihood of off-target editing at these predicted targets was low because all the sites bear at least 1 bp mismatch in the PAM-proximal half of the protospacer. With 4 bp mismatches, 79 and 74 potential targets in human and mice, respectively, were returned. Since the study was performed in mice, 10 top-scored genomic sites (two with 3 bp mismatches; seven with 4 bp mismatches) were amplified from mice with highest on-target base installation followed by Sanger sequencing. None of these sites exhibited detectable editing.

Collectively, these data provided evidence for minimal intergenic deletion and high fidelity of the in vivo base editing system.

### Example 11. Further Description Regarding Base Editor Embodiments

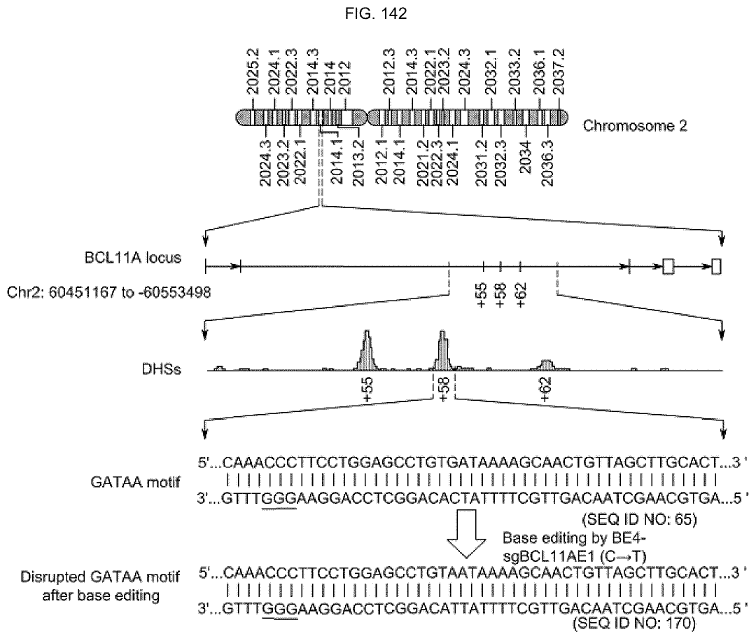

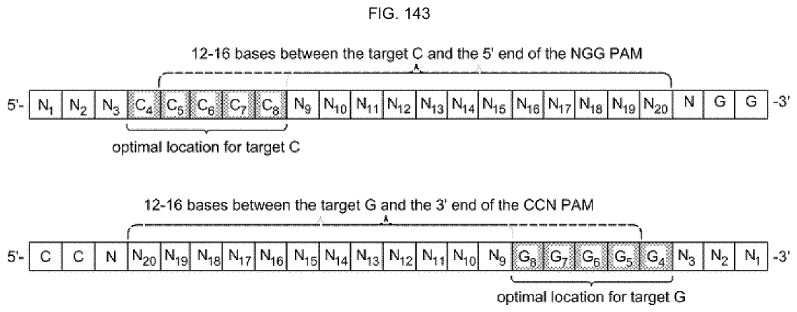

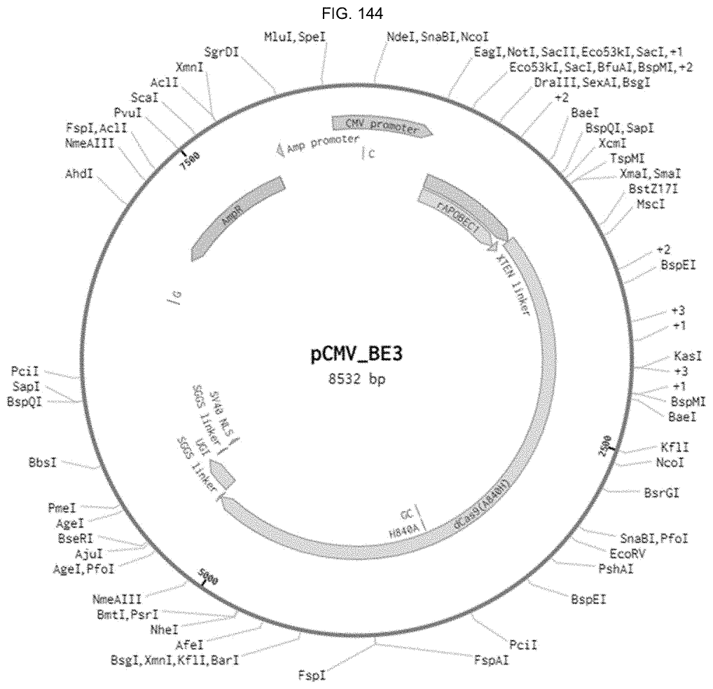

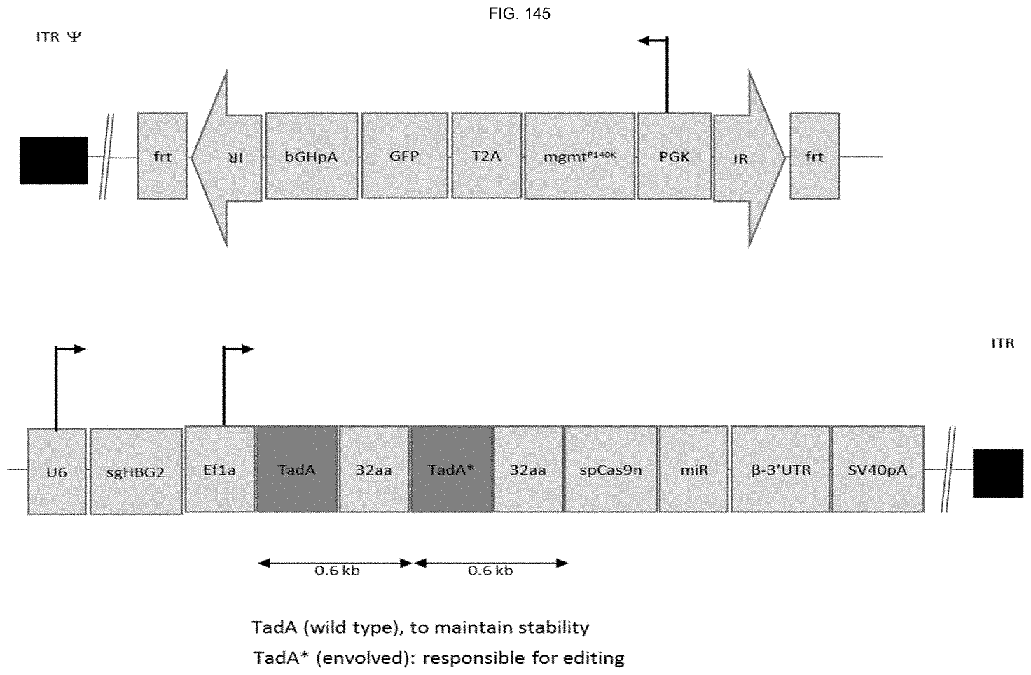

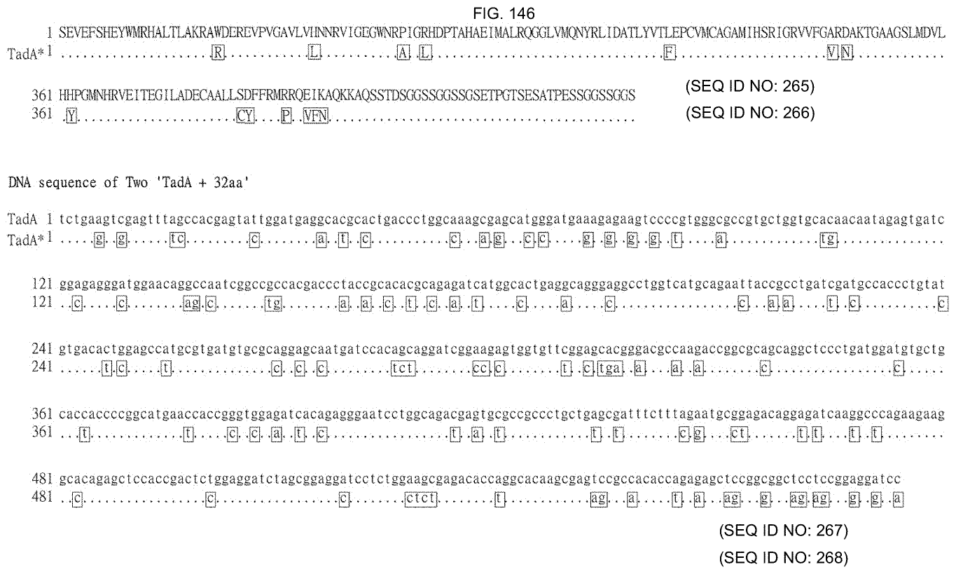

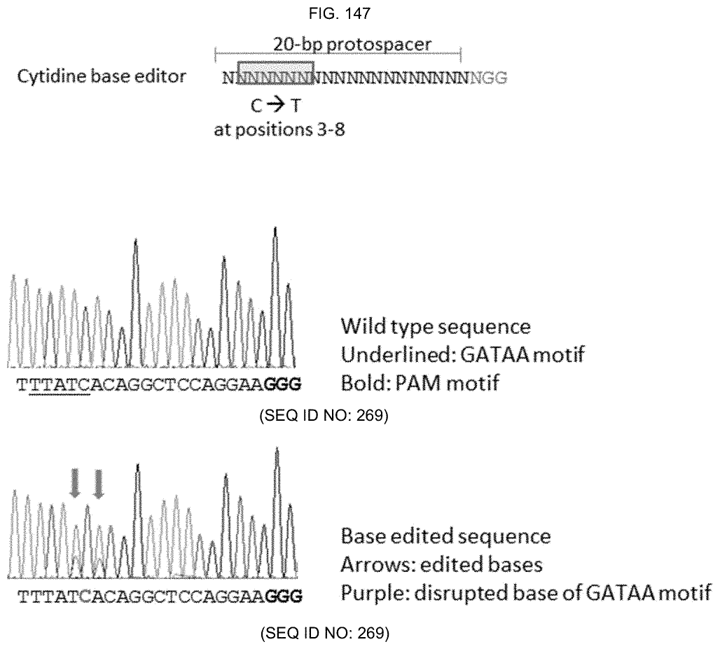

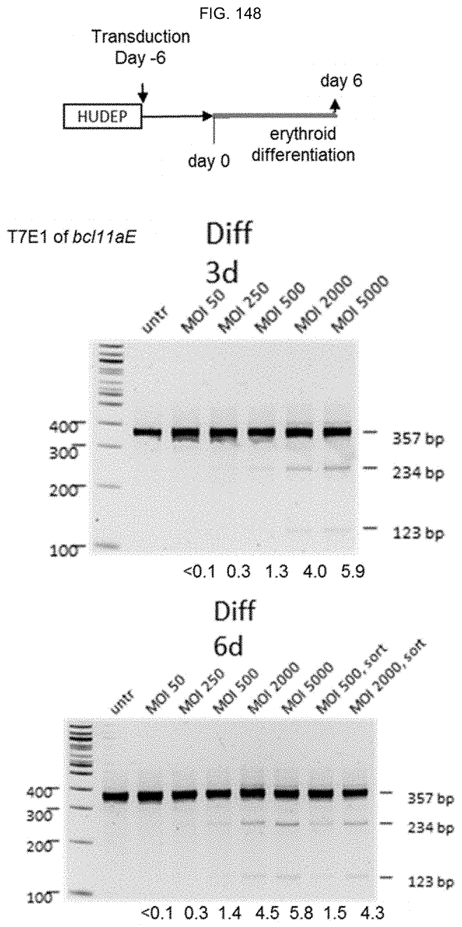

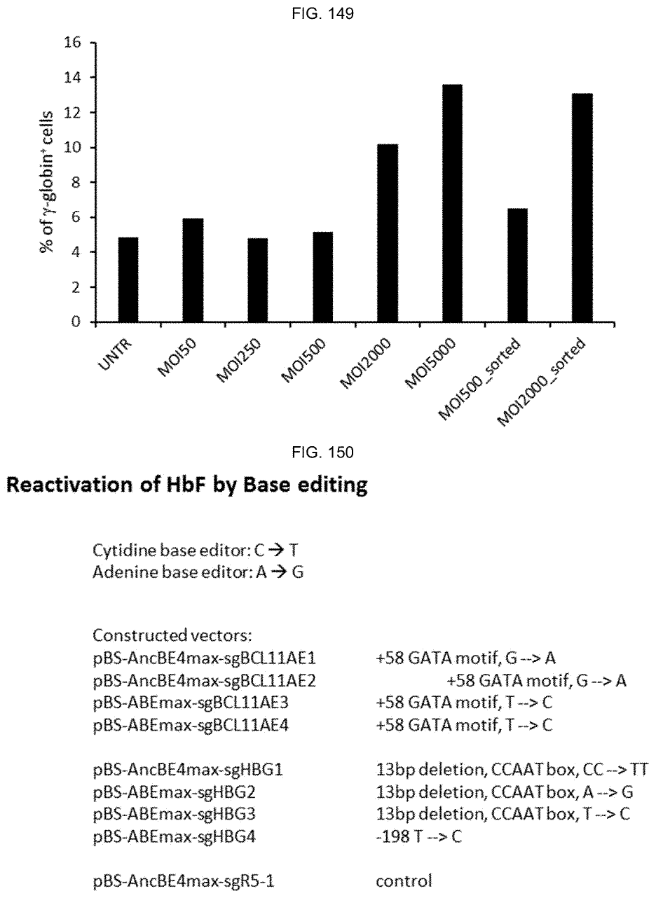

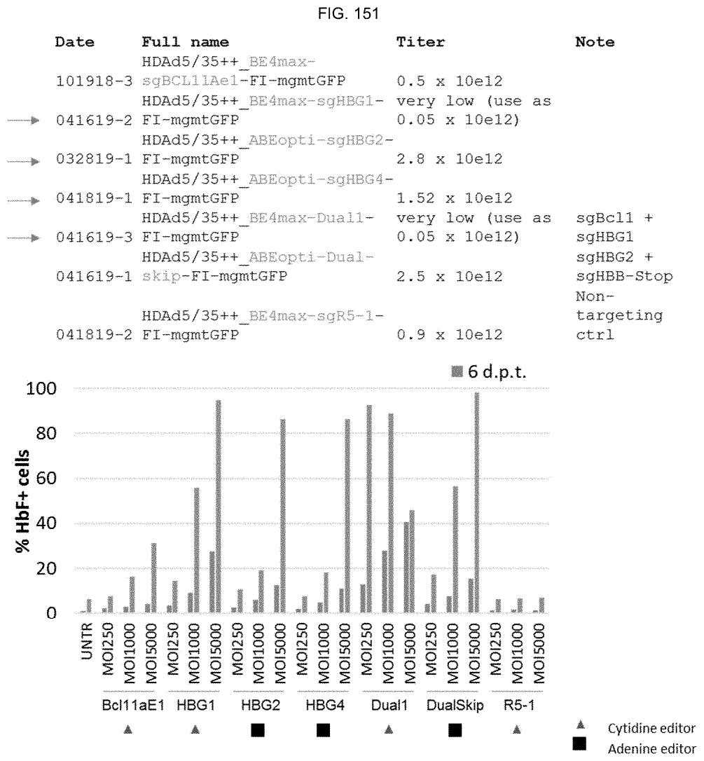

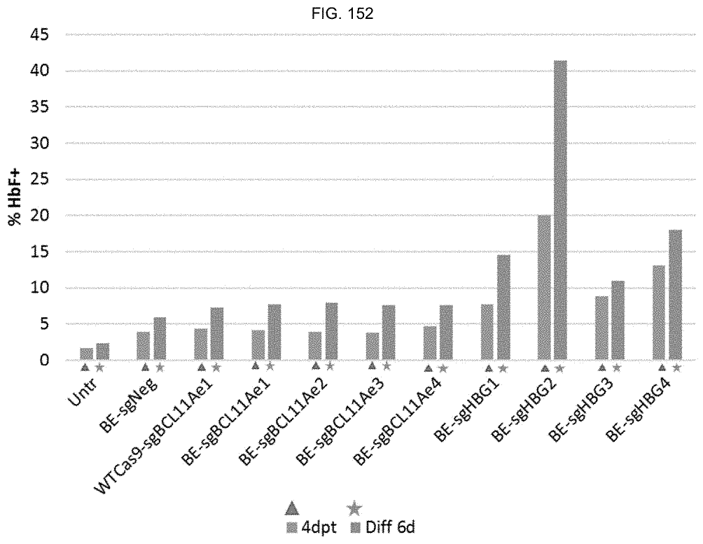

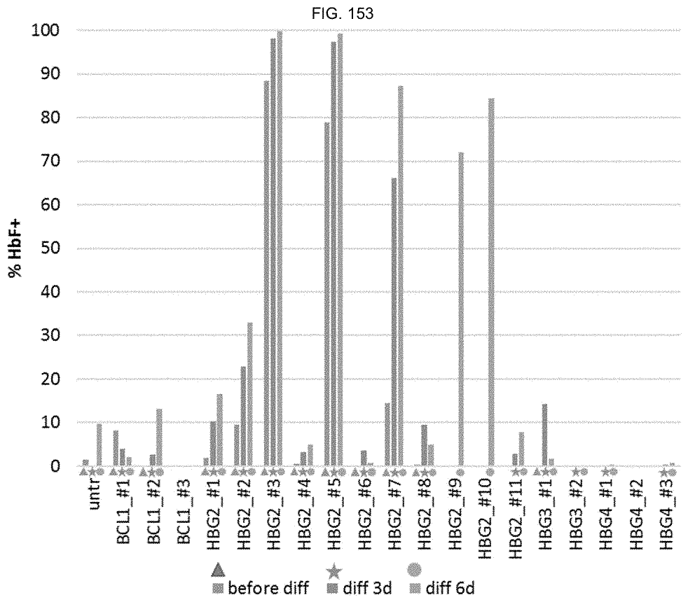

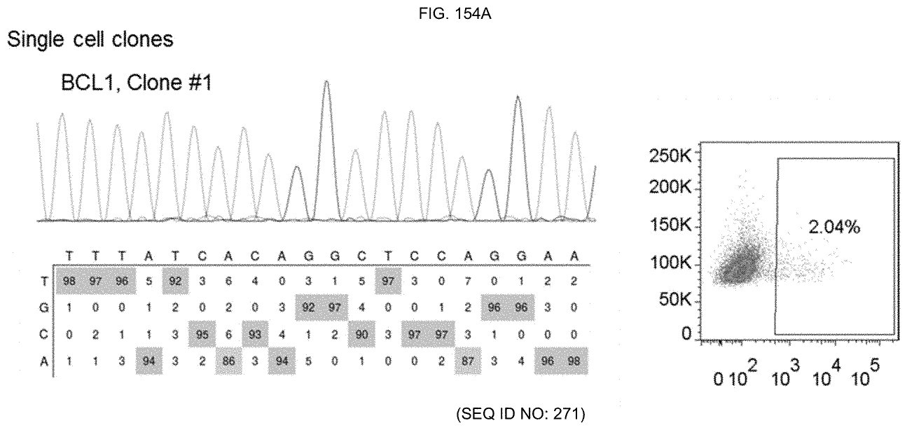

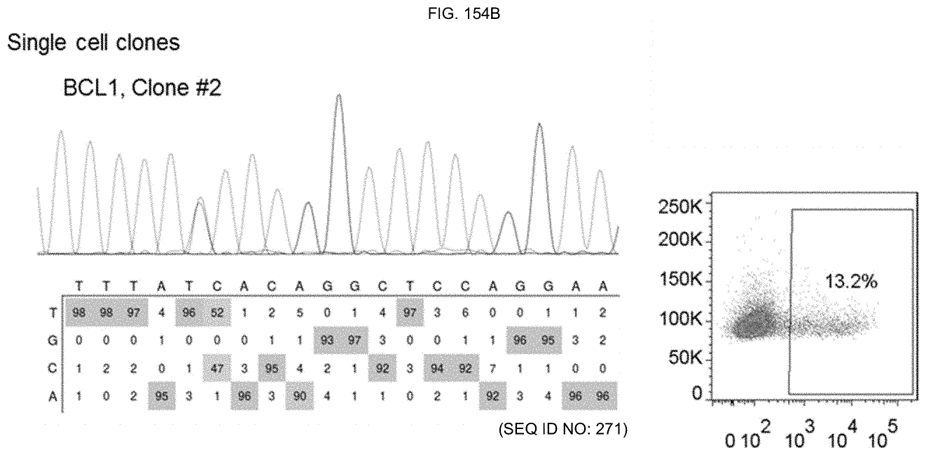

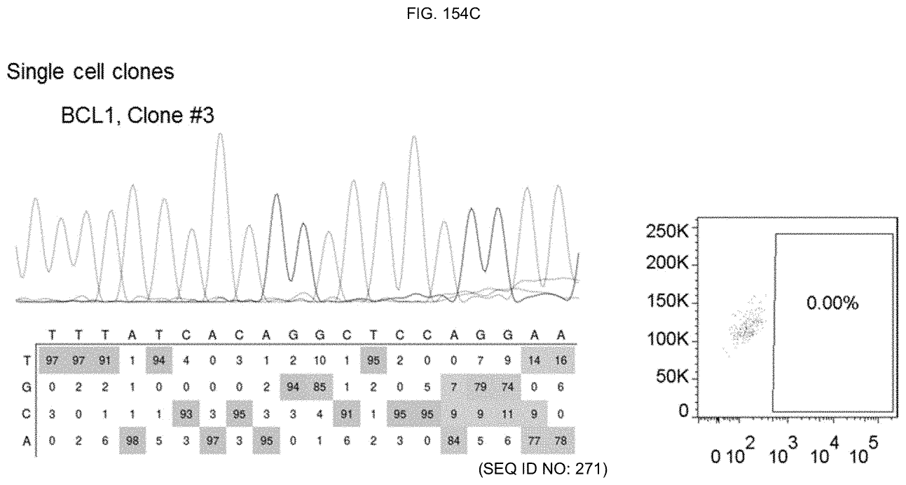

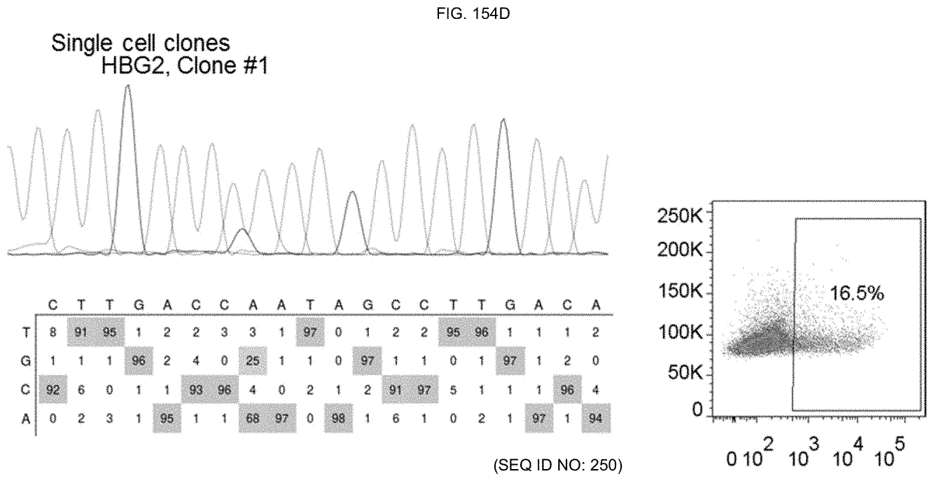

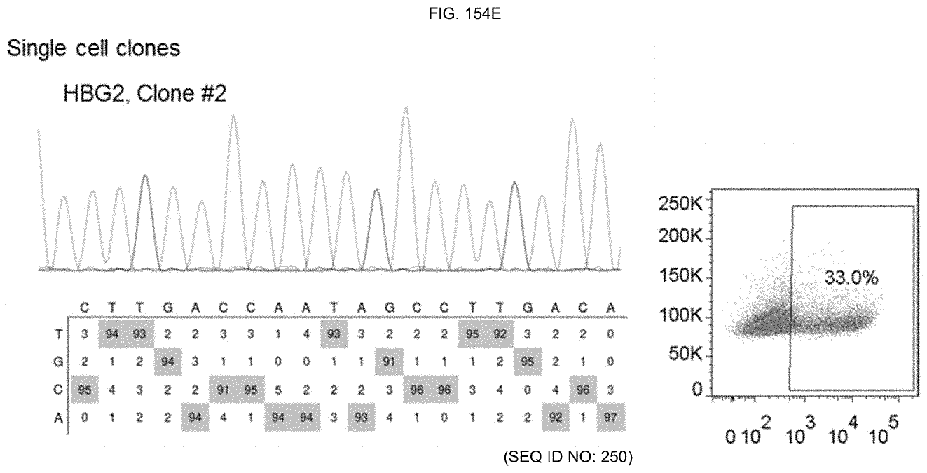

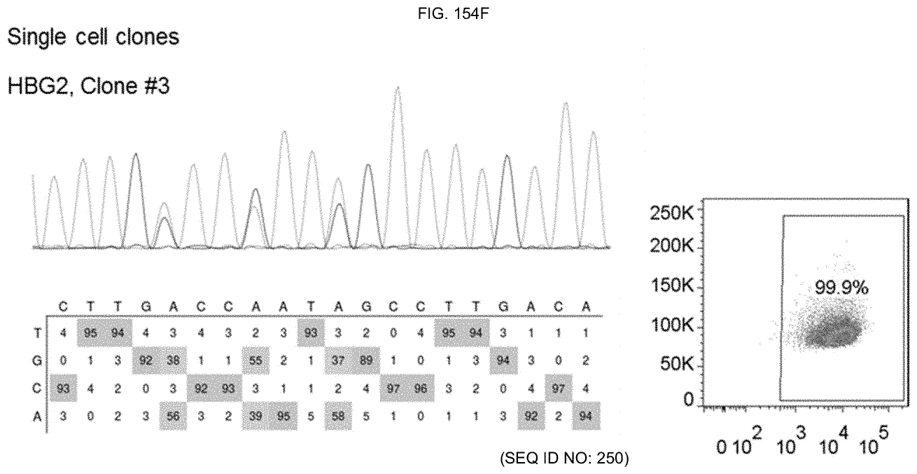

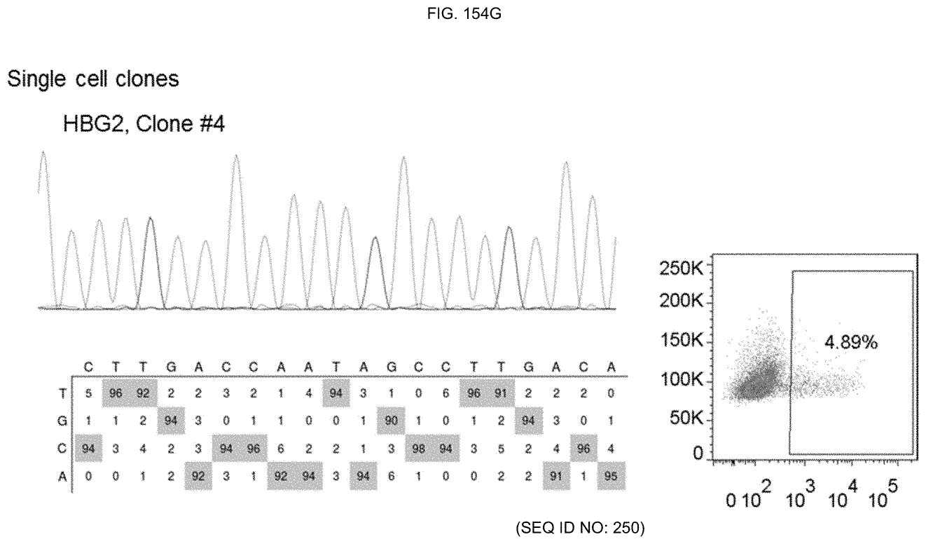

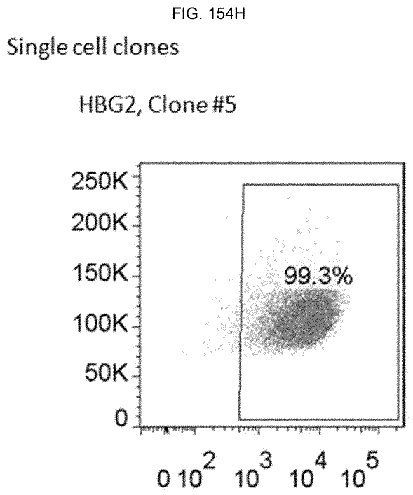

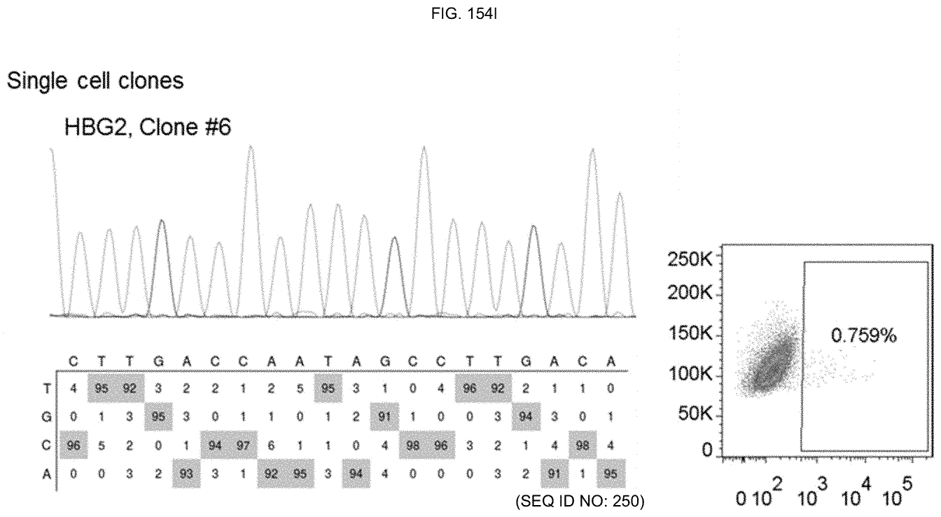

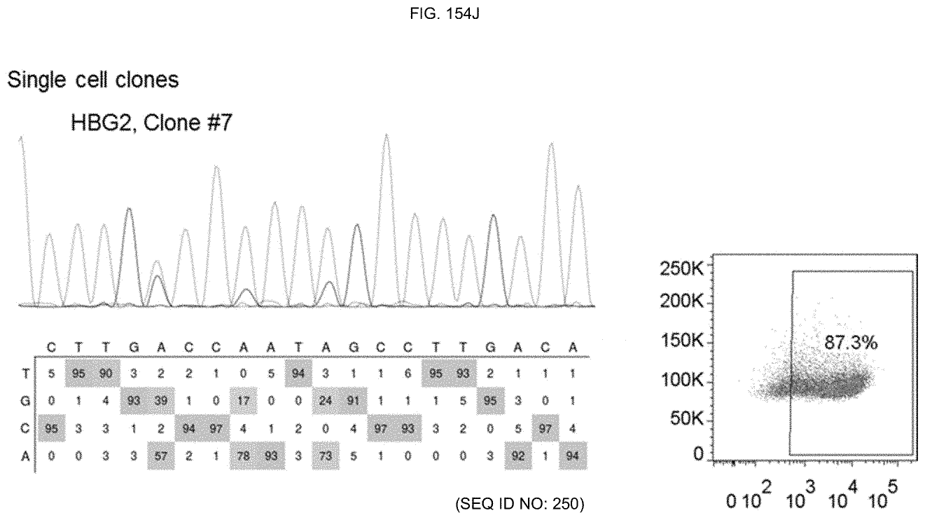

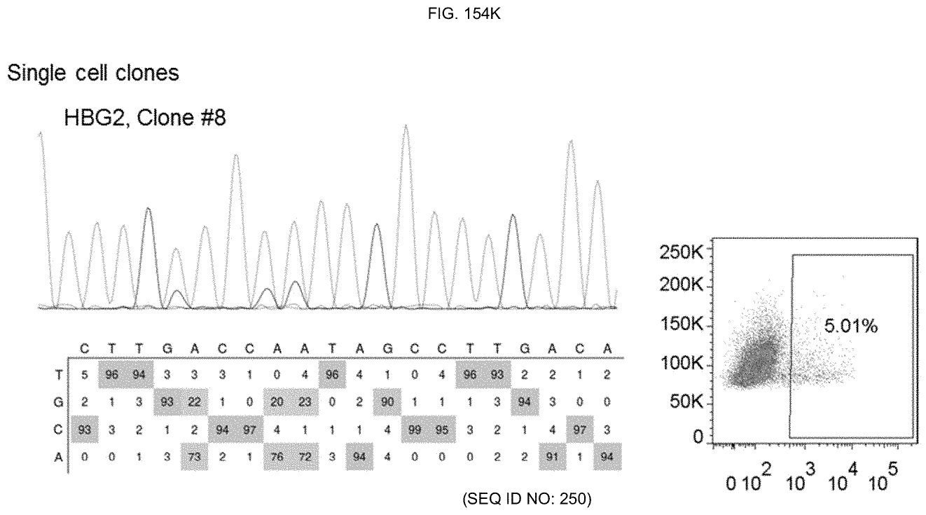

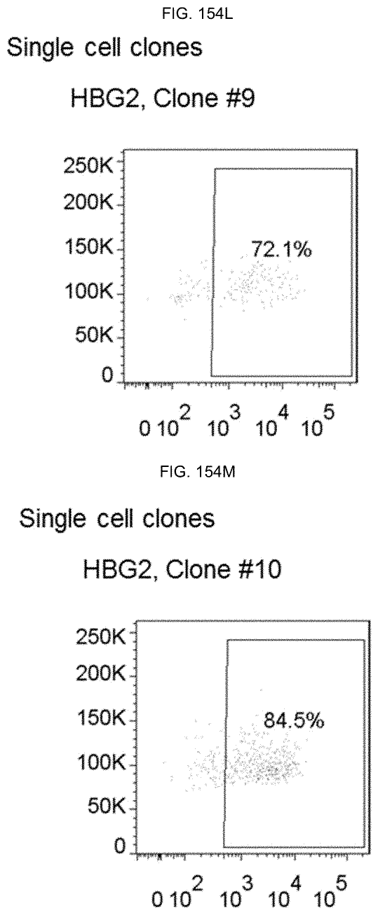

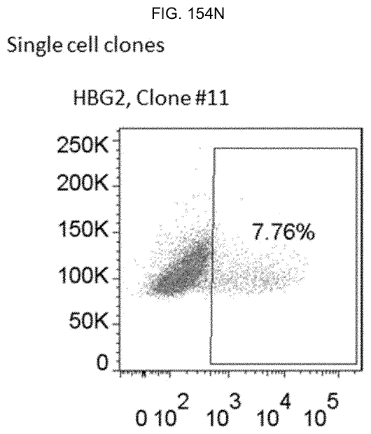

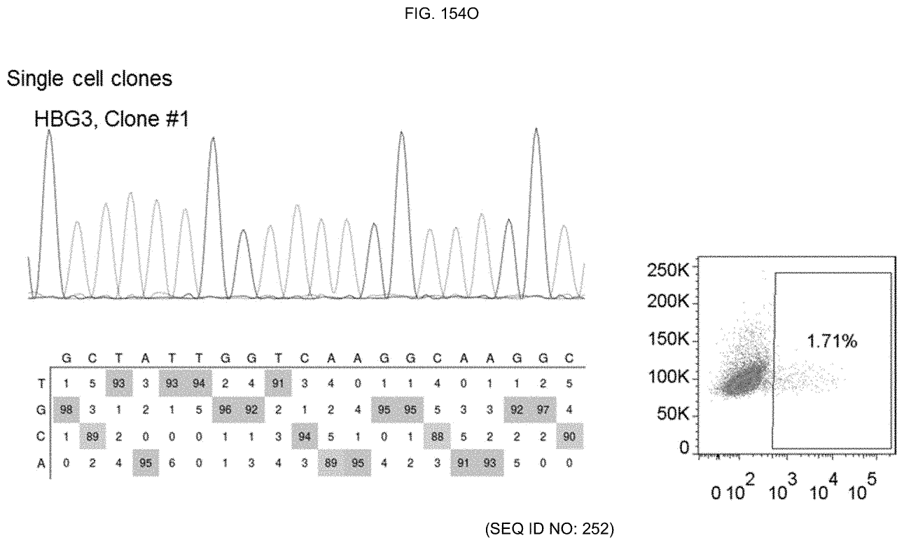

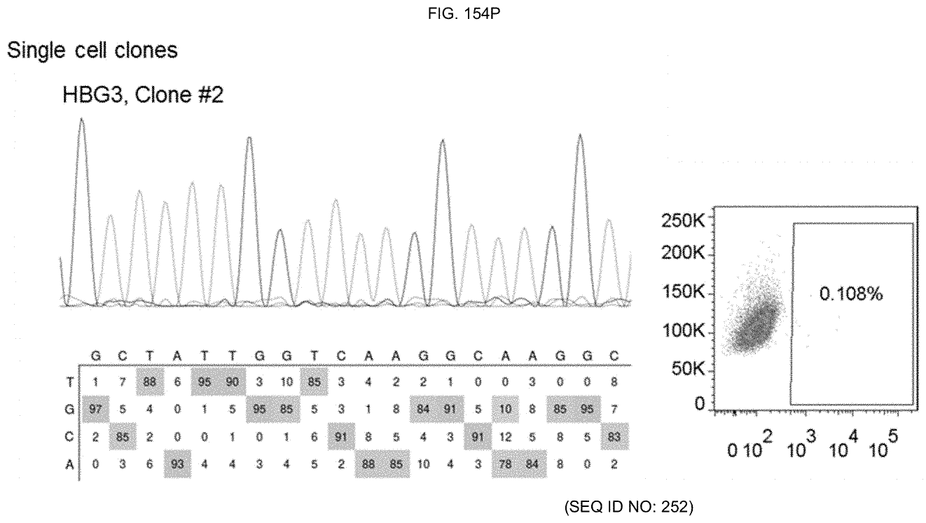

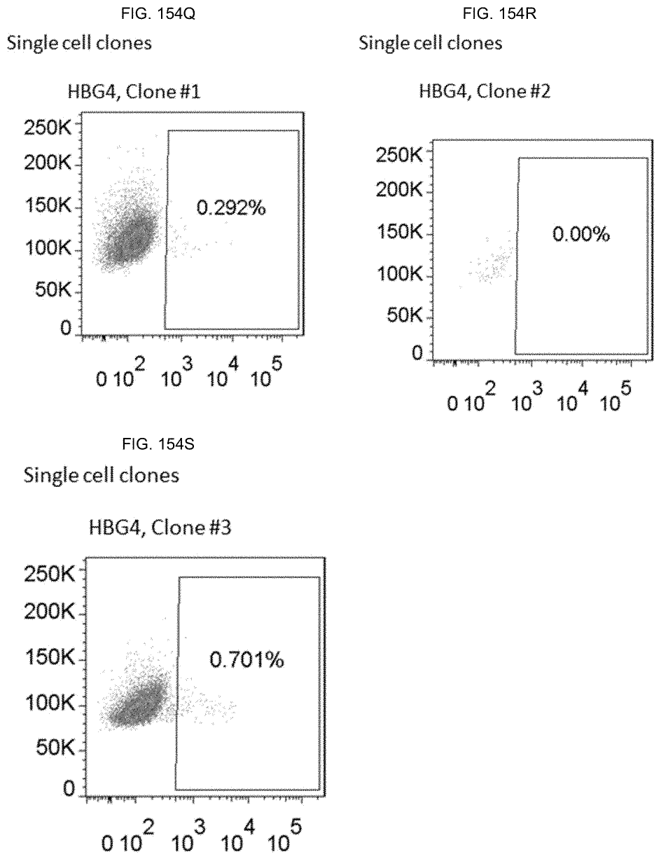

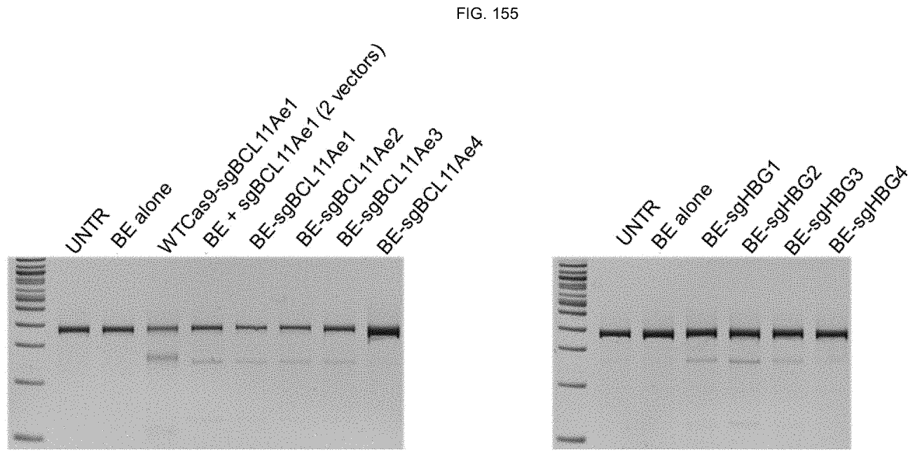

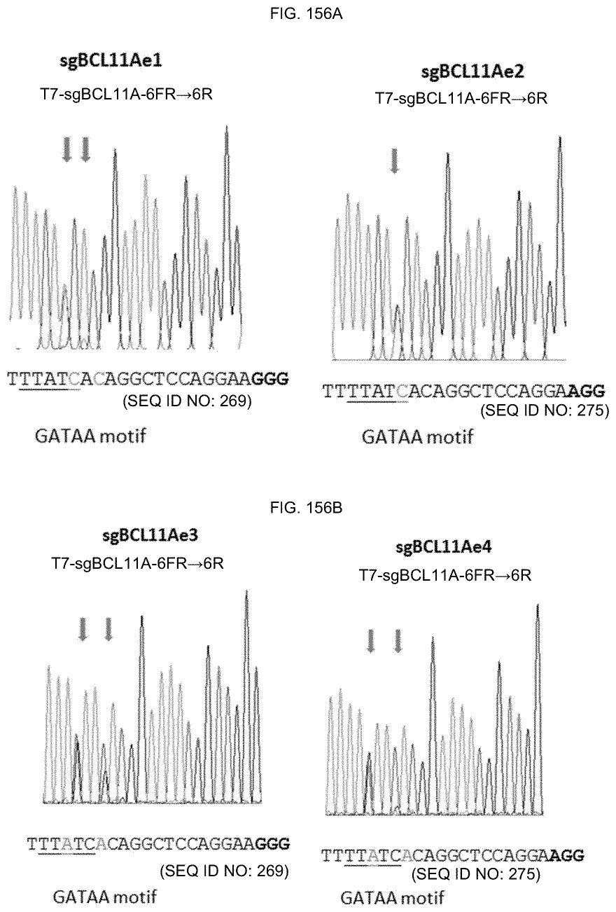

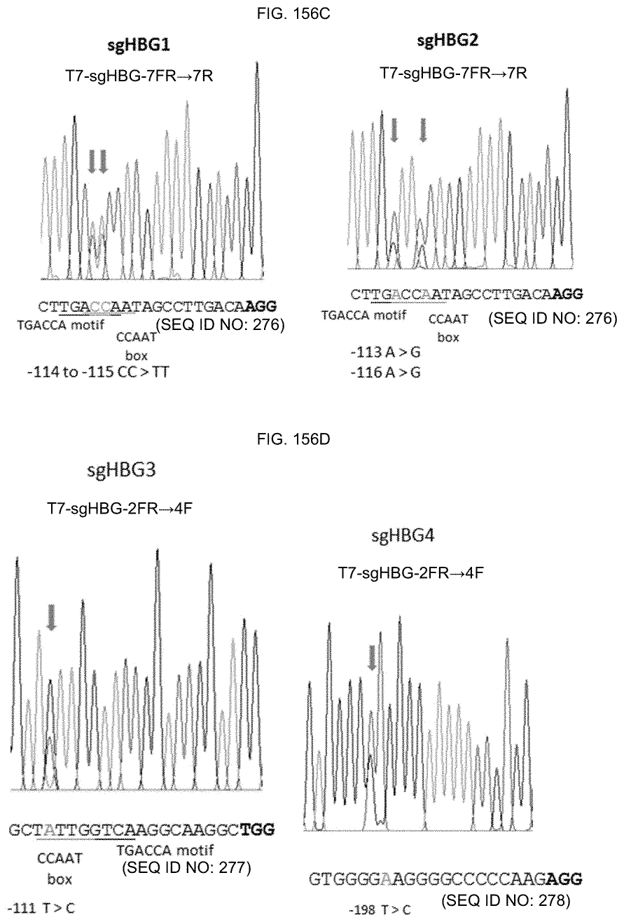

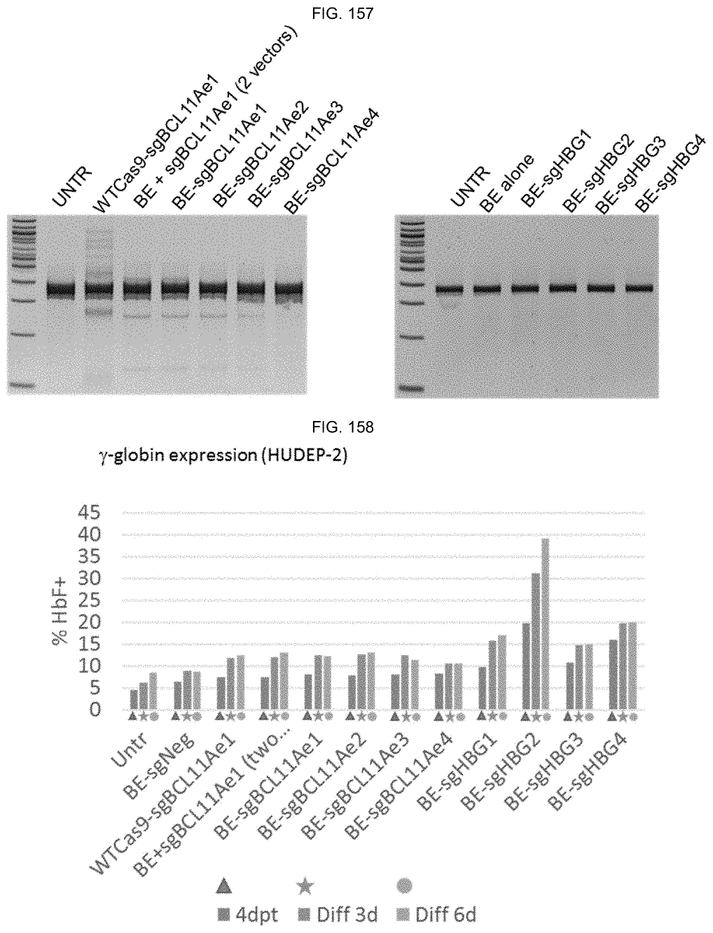

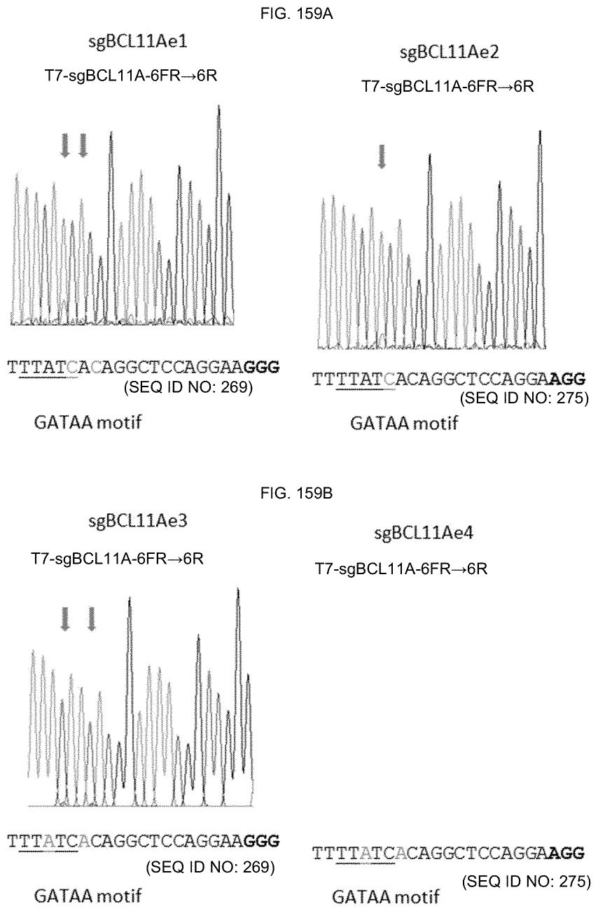

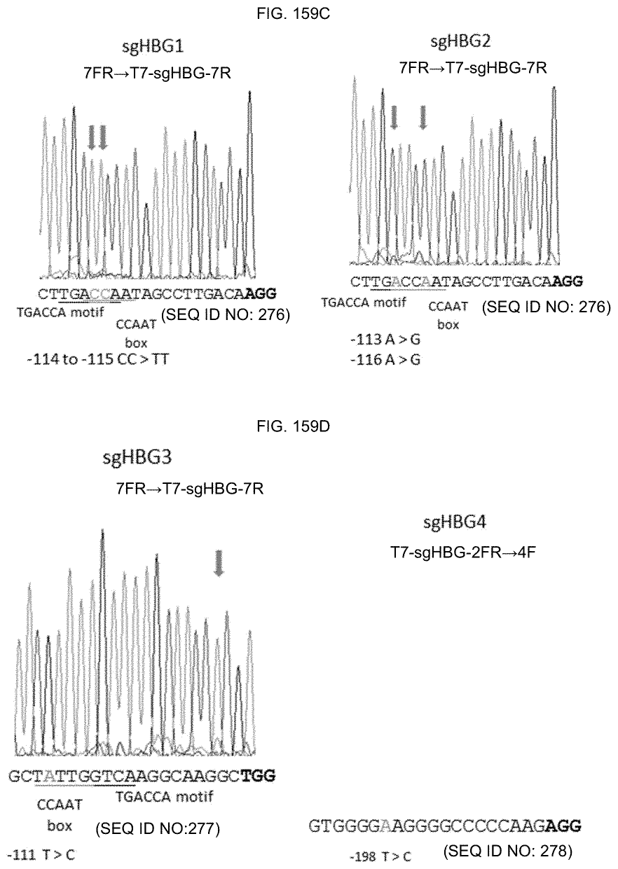

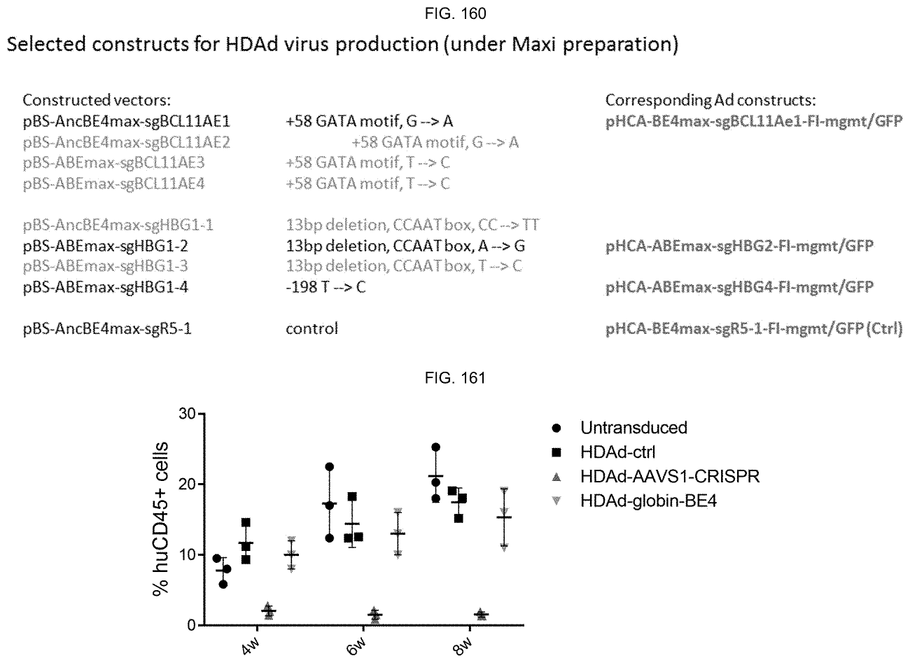

FIG. 141 presents the safety profile of a base editor, including hematology analysis (FIG. 141A) and cellular comparison in bone marrow MNCs (FIG. 141B). An illustration of editing expected to result from activity of base editor BE4-sgBCL11AE1 is shown in FIG. 142. FIG. 143 shows an optimal protospacer sequence arrangement for maximizing base editing efficiency when effecting C to T (top image) or G to A (bottom image) base transformations. FIG. 144 shows a vector for C to T editing when the target C is in positions 4 through 8 within the protospacer. FIG. 145 shows a diagram of viral gDNA (HBG2-miR, adenine editor) which represents a single contiguous construct but has been divided into two sections solely for ease of presentation. FIG. 146 shows sequences of TadA and TadA*. Sanger sequencing was performed to confirm base editing of sequences (FIG. 147). FIG. 148 shows base editing by an HDAd5/35++_BE4-sgBCL11Ae1-Fl-mgmtGFP (041318-1) virus, and FIG. 149 shows the percentage of γ-globin⁺ cells at indicated MOIs. FIG. 150 shows cytidine base editors and adenine base editors for reactivation of HbF by base editing. FIG. 151 shows exemplary base editors and percent HbF+ cells at various MOIs of the base editors. FIG. 152 shows the % HbF+from a second trial in HUDEP-2 cells. FIG. 153 shows results in single-cell derived clones. FIGS. 154A-154S, show data representing individual single-cell derived clones. Base editors were also tested in 293FT cells (FIG. 155). FIGS. 156A-156D, show sanger sequencing results. Base editors were also tested in HUDEP-2 cells (FIG. 157). Expression of γ-globin is shown in FIG. 158. FIGS. 159A-159D, show sanger sequencing results, where available. Constructs were selected for Maxi preparation as shown in FIG. 160.

Engraftment of huCD45+ cells edited, e.g., with HDAd-AAVSI-CRISPR or HDAd-globin-BE4 base editor are shown in FIG. 161.

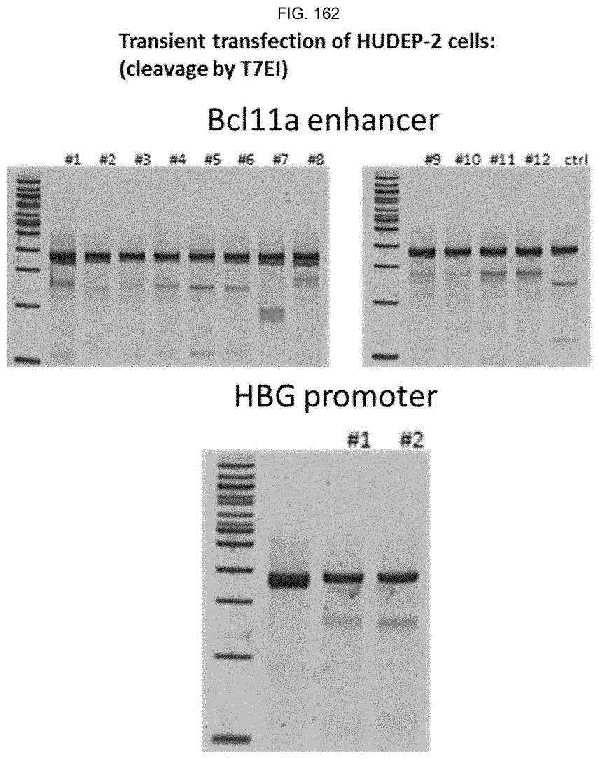

Transient transfection of HUDEP-2 cells (with cleavage by T7EI) is shown in FIG. 162.

Non-limiting examples of base editing constructs for HbF could include (1) pHCA-ABEmax-sgHBG2-miR-FI-mgmtGFP; (2) pHCA-ABEmax-sgHBG4-miR-FI-mgmtGFP; or (3) pHCA-ABEmax-Dual-Skip-miR-FI-mgmtGFP.

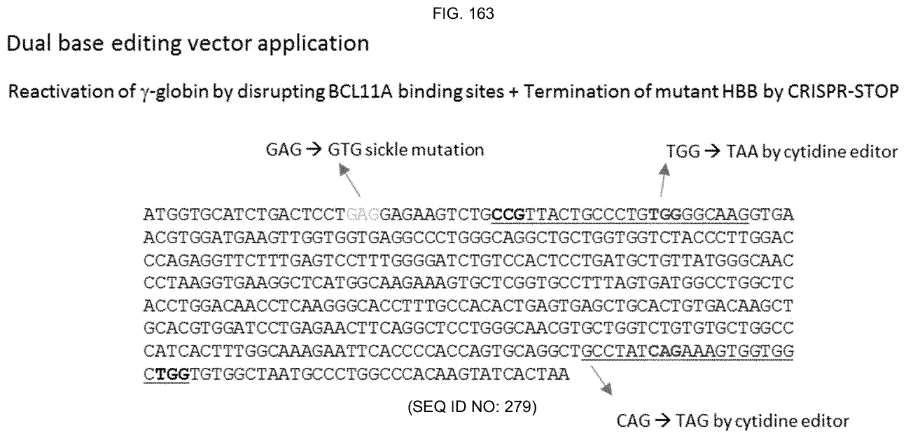

At least one application of base editors includes dual base editing vectors, which application is exemplified in FIG. 163.

In single-cell derived clones, monoallelic or biallelic target base conversion conferred 100% of HbF-positive cells. 60%-113 A to G HPFH mutation in HBG1/2 promoter of mixed HUDEP-2 cells was observed using an ABE vector HDAd-ABE-HBG #2 (see FIG. 135). This vector was chosen for certain further animal studies. Animal studies were carried out in mice that carry 248 kb of the human β-globin locus (β-YAC mice) and thus accurately reflect globin switching (see, e.g., FIG. 137). An EF1α-mgmt^{P140K} expression cassette flanked by FRT and transposon sites was included in the vector for allowing in vivo selection of transduced cells (see, e.g., FIG. 136). After in vivo transduction with HDAd-ABE-HBG #2+HDAd-SB and selection with low doses of O⁶BG/BCNU, an average of 35% of HbF-positive cells was measured in peripheral red blood cells (FIG. 138). In one out of eight mice, a near complete-113 A to G conversion and 90% of HbF-positive cells were achieved. No alterations in blood cell counts were found (FIG. 141). The cellular composition of bone marrow samples was comparable to that of untransduced mice, demonstrating a good safety profile (FIG. 141). Bone marrow lineage minus cells were isolated from primary mice at week 14 after transduction and infused into lethally irradiated C57BL/6J mice. The percentage of HbF-positive cells was maintained in secondary recipients over 16 weeks indicating genome editing occurred in long-term repopulating mouse HSCs. These observations demonstrate that base editors delivered by HDAd5/35++ vectors in vivo are a strategy for precise genome engineering, e.g., for the treatment of hemoglobinopathies.

### VII. CLOSING PARAGRAPHS

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wis.) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gin), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte & Doolittle, J. Mol. Biol. 157(1), 105-32, 1982). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2 M NaH₂PO₄; 0.02 M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al., Molecular Cloning: A Laboratory Manual, 2nd Edition (1989); F. M. Ausubel, et al., eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the terms “about” and “approximately” are used interchangeably herein and have the meaning reasonably ascribed by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in its entirety for their referenced teaching. Where referenced materials are subject to revision over time (e.g., sequence database entries and the like), the content in that reference is incorporated as of the date the reference was included in a filing in the priority claim for this application.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A recombinant adenoviral serotype 35 (Ad35) vector production system comprising:
a recombinant Ad35 helper genome comprising:
a nucleic acid sequence encoding an Ad35 fiber shaft;
a nucleic acid sequence encoding an Ad35 fiber knob; and
recombinase direct repeats (DRs) flanking at least a portion of an Ad35 packaging sequence, and
a recombinant helper dependent Ad35 donor genome comprising:
a 5′ Ad35 inverted terminal repeat (ITR);
a 3′ Ad35 ITR;
an Ad35 packaging sequence; and
a nucleic acid sequence encoding at least one heterologous expression product.

2. A recombinant adenoviral serotype 35 (Ad35) helper vector comprising:
an Ad35 fiber shaft;
an Ad35 fiber knob; and
an Ad35 genome comprising recombinase direct repeats (DRs) flanking at least a portion of an Ad35 packaging sequence.

3. A recombinant adenoviral serotype 35 (Ad35) helper genome comprising:
a nucleic acid sequence encoding an Ad35 fiber shaft;
a nucleic acid sequence encoding an Ad35 fiber knob; and
recombinase direct repeats (DRs) flanking at least a portion of an Ad35 packaging sequence.

4. A recombinant helper dependent adenoviral serotype 35 (Ad35) donor vector comprising:
a nucleic acid sequence comprising
a 5′ Ad35 inverted terminal repeat (ITR);
a 3′ Ad35 ITR;
an Ad35 packaging sequence; and
a nucleic acid sequence encoding at least one heterologous expression product,
wherein the genome does not comprise a nucleic acid sequence encoding an Ad35 viral structural protein; and
an Ad35 fiber shaft and/or an Ad35 fiber knob.

5. A recombinant helper dependent adenoviral serotype 35 (Ad35) donor genome comprising:
a 5′ Ad35 inverted terminal repeat (ITR);
a 3′ Ad35 ITR;
an Ad35 packaging sequence; and
a nucleic acid sequence encoding at least one heterologous expression product, wherein the Ad35 donor genome does not comprise a nucleic acid sequence encoding an expression product encoded by the wild-type Ad35 genome.

6. A method of producing a recombinant helper dependent adenoviral serotype 35 (Ad35) donor vector, the method comprising isolating the recombinant helper dependent Ad35 donor vector from a culture of cells, wherein the cells comprise:
a recombinant Ad35 helper genome comprising:
a nucleic acid sequence encoding an Ad35 fiber shaft;
a nucleic acid sequence encoding an Ad35 fiber knob; and
recombinase direct repeats (DRs) flanking at least a portion of an Ad35 packaging sequence, and
a recombinant helper dependent Ad35 donor genome comprising:
a 5′ Ad35 inverted terminal repeat (ITR);
a 3′ Ad35 ITR;
an Ad35 packaging sequence; and
a nucleic acid sequence encoding at least one heterologous expression product.

7. A recombinant adenoviral serotype 35 (Ad35) production system comprising:
a recombinant Ad35 helper genome comprising:
a nucleic acid sequence encoding an Ad35 fiber shaft;
a nucleic acid sequence encoding an Ad35 fiber knob; and
recombinase direct repeats (DRs) within 550 nucleotides of the 5′ end of the Ad35 genome that functionally disrupt the Ad35 packaging signal but not the 5′ Ad35 inverted terminal repeat (ITR), and
a recombinant Ad35 donor genome comprising:
a 5′ Ad35 ITR;
a 3′ Ad35 ITR;
an Ad35 packaging sequence; and
a nucleic acid sequence encoding at least one heterologous expression product.

8. A recombinant adenoviral serotype 35 (Ad35) helper vector comprising:
an Ad35 fiber shaft;
an Ad35 fiber knob; and
an Ad35 genome comprising recombinase direct repeats (DRs) within 550 nucleotides of the 5′ end of the Ad35 genome that functionally disrupt the Ad35 packaging signal but not the 5′ Ad35 inverted terminal repeat (ITR).

9. A recombinant adenoviral serotype 35 (Ad35) helper genome comprising:
a nucleic acid sequence encoding an Ad35 fiber shaft;
a nucleic acid sequence encoding an Ad35 fiber knob; and
recombinase direct repeats (DRs) within 550 nucleotides of the 5′ end of the Ad35 genome that functionally disrupt the Ad35 packaging signal but not the 5′ Ad35 inverted terminal repeat (ITR).

10. A method of producing a recombinant helper dependent adenoviral serotype 35 (Ad35) donor vector, the method comprising isolating the recombinant helper dependent Ad35 donor vector from a culture of cells, wherein the cells comprise:
a recombinant Ad35 helper genome comprising:
a nucleic acid sequence encoding an Ad35 fiber shaft;
a nucleic acid sequence encoding an Ad35 fiber knob; and
recombinase direct repeats (DRs) within 550 nucleotides of the 5′ end of the Ad35 genome that functionally disrupt the Ad35 packaging signal but not the 5′ Ad35 inverted terminal repeat (ITR), and
a recombinant Ad35 donor genome comprising:
a 5′ Ad35 ITR;
a 3′ Ad35 ITR;
an Ad35 packaging sequence; and
a nucleic acid sequence encoding at least one heterologous expression product.

11. The recombinant Ad35 vector production system, helper vector, helper genome, donor vector, or method of any one of claim 1-4 or 6-10, wherein:
the Ad35 fiber knob is a wild-type Ad35 fiber knob, or
the Ad35 fiber knob is an engineered Ad35 fiber knob, wherein the engineered fiber knob comprises a mutation that increases affinity of the fiber knob with CD46.

12. The recombinant Ad35 vector production system, helper vector, helper genome, donor vector, or method of claim 11, wherein the mutation:
comprises a mutation selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or comprises each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

13. The recombinant Ad35 vector production system, donor genome, donor vector, or method of any one of claim 1, 4-7, or 10, wherein the heterologous expression product comprises a therapeutic expression product operably linked with a regulatory sequence, optionally wherein the therapeutic expression product comprises:
(a) a β-globin protein or γ-globin protein;
(b) an antibody or an immunoglobulin chain thereof, optionally wherein the antibody is an anti-CD33 antibody;
(c) a first antibody or an immunoglobulin chain thereof and a second antibody or an immunoglobulin chain thereof, optionally wherein the antibody is an anti-CD33 antibody;
(d) a CRISPR-associated RNA-guided endonuclease and/or a guide RNA (gRNA), optionally wherein the CRISPR-associated RNA-guided endonuclease comprises Cas9 or cpf1;
(e) a base editor and/or a gRNA, optionally wherein the base editor is a cytosine base editor (CBE) or adenine base editor (ABE), optionally wherein the base editor comprises a catalytically disabled nuclease selected from a disabled Cas9 and a disabled cpf1;
(f) a coagulation factor or a protein that blocks or reduces viral infection, optionally wherein the therapeutic expression produce comprises a Factor VII replacement protein or a Factor VIII replacement protein;
(g) a checkpoint inhibitor;
(h) chimeric antigen receptor or engineered T cell receptor; or
(i) a protein selected from the group consisting of γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, FancA, FancB, FancC, FancD1, FancD2, FancE, FancF, FancG, Fancl, FancJ, FancL, FancM, FancN, FancO, FancP, FancQ, FancR, FancS, FancT, FancU, FancV, FancW, soluble CD40, CTLA, Fas L, an antibody to PD-L1, an antibody to CD4, an antibody to CD5, an antibody to CD7, an antibody to CD52, an antibody to IL-1, an antibody to IL-2, an antibody to IL-4, an antibody to IL-6, an antibody to IL-10, an antibody to TNF, an antibody to a TCR specifically present on autoreactive T cells, a globin family gene, WAS, phox, dystrophin, pyruvate kinase, CLN3, ABCD1, arylsulfatase A, SFTPB, SFTPC, NLX2.1, ABCA3, GATA1, a ribosomal protein gene, TERT, TERC, DKC1, TINF2, CFTR, LRRK2, PARK2, PARK7, PINK1, SNCA, PSEN1, PSEN2, APP, SOD1, TDP43, FUS, ubiquilin 2, and/or C9ORF72, optionally wherein the protein is a FancA protein.

14. The recombinant Ad35 vector production system, donor genome, donor vector, or method of claim 13(d) or 13(e), wherein:
the gRNA binds a target nucleic acid sequence of HBG1, HBG2, and/or erythroid enhancer bcl11a, optionally wherein the gRNA is engineered to increase expression of γ-globin; or
the gRNA binds a target nucleic acid sequence that encodes a portion of CD33, optionally wherein the CD33 is human CD33.

15. The recombinant Ad35 vector production system, donor genome, donor vector, or method of claim 13, wherein the therapeutic expression product comprises:
a β-globin protein or a γ-globin protein; and
a CRISPR system comprising a CRISPR-associated RNA-guided endonuclease; and one, two, or three of:
a gRNA that binds a target nucleic acid sequence of HBG1;
a gRNA that binds a target nucleic acid sequence of HBG2; and/or
a gRNA that binds a target nucleic acid sequence of Bcl11a,
optionally wherein the gRNA is engineered to increase expression of γ-globin.

16. The recombinant Ad35 vector production system, donor genome, donor vector, or method of claim 13, wherein the regulatory sequence(s) comprise a promoter, optionally wherein the promoter is a β-globin promoter, optionally wherein the β-globin promoter has a length of about 1.6 kb and/or comprises a nucleic acid according to positions 5228631-5227023 of chromosome 11.

17. The recombinant Ad35 vector production system, donor genome, donor vector, or method of claim 13, wherein the regulatory sequence(s) comprise a Locus Control Region (LCR), optionally wherein the LCR is a β-globin LCR

18. The recombinant Ad35 vector production system, donor genome, donor vector, or method of claim 13, wherein the β-globin LCR:
comprises β-globin LCR DNAse I hypersensitive sites (HS) comprising or consisting of HS1, HS2, HS3, and HS4, optionally wherein the β-globin LCR has a length of about 4.3 kb; comprises β-globin LCR DNAse I HS comprising HS1, HS2, HS3, HS4, and HS5, optionally wherein the β-globin LCR has a length of about 21.5 kb; or
wherein the β-globin LCR comprises a sequence according to positions 5292319-5270789 of chromosome 11.

19. The recombinant Ad35 vector production system, donor genome, donor vector, or method of claim 13 or 14, wherein the regulatory sequence(s) comprise a 3′HS1, optionally wherein the 3′HS1 comprises a sequence according to positions 5206867-5203839 of chromosome 11.

20. The recombinant Ad35 vector production system, donor genome, donor vector, or method of claim 13, wherein the regulatory sequence(s) comprise an miRNA binding site, optionally wherein:
the miRNA binding site is a binding site for an miRNA naturally expressed by a species of interest;
the miRNA demonstrates differential occupancy profiles in the blood and a tumor microenvironment or target tissue, optionally wherein the occupancy profile is higher in blood than in the tumor microenvironment or target tissue;
the miRNA binding site comprises an miR423-5, miR423-5p, miR42-2, miR181c, miR125a, or miR15a binding sites; and/or
the miRNA binding sites comprise an miR187 or miR218 binding sites.

21. The recombinant Ad35 vector production system, donor genome, donor vector, or method of any one of claim 1, 4-7, or 10, wherein the nucleic acid encoding the heterologous expression product is part of a payload further comprising an integration element, optionally wherein the integration element comprises an expression product.

22. The recombinant Ad35 vector production system, donor genome, donor vector, or method of claim 21, wherein the integration element is engineered for integration into a target genome by homologous recombination, wherein the integration element is flanked by homology arms that correspond to contiguously linked sequences of the target genome, optionally wherein:
the homology arms are between 0.8 and 1.8 kb; and/or
the homology arms are homologous to nucleic acid sequences of the target genome that flank a chromosomal safe harbor locus, optionally wherein the safe harbor loci is selected from AAVS1, CCR5, HPRT, or Rosa.

23. The recombinant Ad35 vector production system, donor genome, donor vector, or method of claim 21, wherein the integration element is engineered for integration into a target genome by transposition, wherein the integration element is flanked by transposon inverted repeats (IRs), optionally wherein the transposon IRs are flanked by recombinase DRs.

24. The recombinant Ad35 vector production system, donor genome, donor vector, or method of claim 23, wherein:
the transposon IRs are Sleeping Beauty (SB) IRs, optionally wherein the SB IRs are pT4 IRs; or
the transposon IRs are piggyback, Mariner, frog prince, Tol2, TcBuster, or spinON IRs.

25. The recombinant Ad35 vector production system, donor genome, donor vector, or method of any one of claim 21, comprising a nucleic acid encoding a transposase that mediates transposition of the integration element flanked by the transposon IRs, optionally wherein the nucleic acid encoding the transposase is comprised by a support vector or support vector genome.

26. The recombinant Ad35 vector production system, donor genome, donor vector, or method of claim 25, wherein the transposase is a Sleeping beauty, piggyback, Mariner, frog prince, Tol2, TcBuster, or spinON transposase, optionally wherein the transposase is a Sleeping Beauty 100x (SB100x) transposase.

27. The recombinant Ad35 vector production system, donor genome, donor vector, or method of claim 25 or 26, wherein the nucleic acid encoding the transposase is operably linked with a PGK promoter.

28. The recombinant Ad35 vector production system, helper vector, helper genome, or method of any one of claim 1-3 or 6-10, wherein the recombinase DRs that flank at least a portion of the Ad35 packaging sequence and/or are within 550 nucleotides of the 5′ end of the Ad35 genome and functionally disrupt the Ad35 packaging signal but not the 5′ Ad35 ITR are FRT, loxP, rox, vox, AttB, or AttP sites.

29. The recombinant Ad35 vector production system, helper vector, helper genome, or method of claim 28, wherein a nucleic acid encoding a recombinase for excision of the at least portion of the Ad35 packaging sequence is encoded by a nucleic acid sequence of a cell comprising the helper genome.

30. The recombinant Ad35 vector production system, helper vector, helper genome, or method of any one of claim 23, wherein the recombinase DRs that flank the transposon IRs are FRT, loxP, rox, vox, AttB, or AttP sites.

31. The recombinant Ad35 vector production system, helper vector, helper genome, or method of any one of claim 21, wherein a nucleic acid encoding a recombinase for excision of the nucleic acid comprising the integration element is comprised by a support vector or support vector genome.

32. The recombinant Ad35 vector production system, helper vector, helper genome, or method of claim 29 or 31, wherein the recombinase is a Flp, Cre, Dre, Vika, or PhiC31 recombinase.

33. The recombinant Ad35 vector production system, helper vector, helper genome, or method of claim 32, wherein the nucleic acid encoding the recombinase is operably linked with an EF1α promoter.

34. The recombinant Ad35 vector production system, helper vector, helper genome, or method of any one of claim 21,
wherein the payload comprises an integration element comprising the heterologous expression product,
wherein the heterologous expression product comprises a β-globin protein operably linked with a β-globin promoter and a β-globin long LCR,
wherein the integration element is flanked by SB IRs,
and wherein the SB IRs are flanked by recombinase DRs, optionally wherein the recombinase DRs are FRT sites.

35. The recombinant Ad35 vector production system, helper vector, helper genome, or method of any one of claim 21, wherein the payload comprises:
an integration element, and
a conditionally expressed nucleic acid sequence that encodes an expression product, is not comprised by the integration element, and is positioned such that it is rendered nonfunctional by integration of the integration element into a target genome.

36. The recombinant Ad35 vector production system, helper vector, helper genome, or method of claim 35, wherein the expression product encoded by the conditionally expressed nucleic acid sequence comprises a CRISPR system component or a base editor system component, optionally wherein the component is a CRISPR-associated RNA-guided endonuclease, a base editor enzyme, or a gRNA.

37. The recombinant Ad35 vector production system, helper vector, helper genome, or method of any one of claim 21, wherein the payload comprises a selection cassette, optionally wherein the selection cassette is comprised by the integration element.

38. The recombinant Ad35 vector production system, helper vector, helper genome, or method of claim 37, wherein the selection cassette comprises a nucleic acid sequence encoding mgmt^{P140K} or wherein the selection cassette comprises a nucleic acid sequence encoding an anti-CD33 shRNA.

39. The recombinant Ad35 vector production system, helper vector, helper genome, or method of any one of claim 1-3 or 6-10, wherein the at least portion of the Ad35 packaging sequence flanked by recombinase DRs corresponds to nucleotides 138-481 of the Ad35 sequence according to GenBank Accession No. AX049983.

40. The recombinant Ad35 vector production system, helper vector, helper genome, or method of any one of claim 1-3 or 6-10, wherein the at least portion of the Ad35 packaging sequence flanked by recombinase DRs corresponds to: nucleotides 179-344; nucleotides 366-481; nucleotides 155-481; nucleotides 159-480; nucleotides 159-446; nucleotides 180-480; nucleotides 207-480; nucleotides 140-446; nucleotides 159-446; nucleotides 180-446; nucleotides 202-446; nucleotides 159-481; nucleotides 180-384; nucleotides 180-481; or nucleotides 207-481 of the Ad35 sequence according to GenBank Accession No. AX049983.

41. The recombinant Ad35 vector production system, helper vector, helper genome, or method of any one of claim 1-3 or 6-10, wherein the recombinase DRs are LoxP sites.

42. The helper vector or helper genome of any one of claim 2, 3, 8, or 9, wherein the Ad35 helper genome comprises Ad5 E4orf6 for amplification in 293 T cells.

43. The helper vector or helper genome of any one of claim 2, 3, 8, or 9, wherein the helper genome comprises or generates the sequence as set forth in any one of SEQ ID NOs: 51-65.

44. A cell comprising the helper vector, the helper genome, the donor vector, or the donor genome of any one of claim 2-5, 8, or 9, optionally wherein the cell is a HEK293 cell.

45. A cell comprising the donor genome of any one of claim 1, 4, 6, 7, 10, 13-27 or 44 optionally wherein the cell is an erythrocyte, optionally wherein the cell is a hematopoietic stem cell, T-cell, B-cell, or myeloid cell, optionally wherein the cell secretes the expression product.

46. The method of claim 6 or 10, wherein the cells are HEK293 cells.

47. A method of modifying a cell, the method comprising contacting the cell with an Ad35 donor vector according to any one of claim 5 or 11-27.

48. A method of modifying a cell of a subject, the method comprising administering to the subject an Ad35 donor vector according to any one of claim 5 or 11-27, optionally wherein the method does not comprise isolation of the cell from the subject.

49. A method of treating a disease or condition in a subject in need thereof, the method comprising administering to the subject an Ad35 donor vector according to any one of claim 5 or 11-27, optionally wherein the administration is intravenous.

50. The method of claim 49, wherein the method comprises administering to the subject a mobilization agent, optionally wherein the mobilization agent comprises one or more of granulocyte-colony stimulating factor, GM-CSF, S-CSF, a CXCR4 antagonist, and a CXCR2 agonist, optionally wherein the CXCR4 antagonist is AMD3100 and/or wherein the CXCR2 agonist is GRO-β.

51. The method of claim 49 or 50, wherein the Ad35 donor vector comprises a selection cassette, optionally wherein the method further comprises administering a selection agent to the subject, optionally wherein the selection cassette encodes mgmt^{P140K} and the selection agent is O⁶BG/BCNU.

52. The method of any one of claim 49, wherein the method further comprises administering to the subject an immune suppression agent, optionally wherein the immune suppression regimen comprises a steroid, an IL-6 receptor antagonist, and/or an IL-1 R receptor antagonist, optionally wherein the steroid comprises a glucocorticoid or dexamethasone.

53. The method of any one of claim 49, wherein the Ad35 donor vector comprises an integration element and the method causes integration and/or expression of a copy of the integration element thereof in at least 20%, 30%, 40%, 50%, 60%, 70%, 80%, 90%, or 95% of cells expressing CD46, in at least 20%, 30%, 40%, 50%, 60%, 70%, 80%, 90%, or 95% of hematopoietic stem cells, and/or in at least 20%, 30%, 40%, 50%, 60%, 70%, 80%, 90%, or 95% of erythroid Ter119⁺ cells.

54. The method of any one of claim 49, wherein the method causes integration of an average of at least 2 copies or at least 2.5 copies of the integration element in target cell genomes comprising at least 1 copy of the integration element.

55. The method of any one of claim 49, wherein the method causes expression of an expression product encoded by the payload or an integration element thereof at a level that is at least about 20% of the level of reference or at least about 25% of the level of a reference, optionally wherein the reference is expression of an endogenous reference protein in the subject or in a reference population.

56. The method of any one of claim 49, wherein the disease or condition is a hemoglobinopathy, a platelet disorder, anemia, an immune deficiency a coagulation factor deficiency, Fanconi anemia, alpha-1 antitrypsin deficiency, sickle cell anemia, thalassemia, thalassemia intermedia, hemophilia A, hemophilia B, von Willebrand Disease, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome, or mucopolysaccharidosis.

57. The method of any one of claim 49, wherein the subject is a subject suffering from cancer and the method treats, prevents, or delays cancer, or delays cancer recurrence,
optionally wherein the subject is a carrier of one or more germ-line mutation associated with development of cancer,
optionally wherein the cancer is anaplastic astrocytoma, breast cancer, ovarian cancer, colorectal cancer, diffuse intrinsic brainstem glioma, Ewing sarcoma, glioblastoma multiforme, malignant glioma, melanoma, metastatic malignant melanoma, nasopharyngeal cancer, or a pediatric cancer,
optionally wherein the subject has received or is administered O⁶BG, TMZ (temozolomide), and/or BCNU (Carmustine).

58. The method of any one of claim 49, wherein the disease or condition is thalassemia intermedia, optionally wherein the vector or genome comprises a nucleic acid encoding one or more expression products selected from:
expression product(s) that increase or reactivate expression of endogenous γ-globin, optionally wherein the expression product(s) that increase or reactivate expression of endogenous γ-globin comprises a CRISPR-associated RNA-guided endonuclease or base editor and one or more of:
a gRNA that binds a nucleic acid sequence of HBG1 and is engineered to increase expression from a coding sequence operably linked with the target nucleic acid sequence;
a gRNA that binds a nucleic acid sequence of HBG2 and is engineered to increase expression from a coding sequence operably linked with the target nucleic acid sequence; and
a gRNA that binds a nucleic acid sequence of erythroid enhancer bcl11a and is engineered to reduce BCL11A expression;
γ-globin; and
β-globin,
optionally wherein the method reduces a symptom of thalassemia intermedia and/or treats thalassemia intermedia and/or increases HbF.
